(19) United States
(12) Patent Application Publication
(10) Pub. No.: US 2024/0116892 A1
(43) Pub. Date: Apr. 11, 2024

(54) HETEROCYCLIC AMIDE AND UREA COMPOUNDS AS JAK2 INHIBITORS

(71) Applicant: Ajax Therapeutics, Inc., New York, NY (US)

(72) Inventors: Craig E. Masse, Cambridge, MA (US); Kevin Robert DeMarco, Cotati, CA (US); Jiayi Xu, Marlboro, NJ (US); Jeremy R. Greenwood, Brooklyn, NY (US); Phani Ghanakota, Edison, NJ (US)

(21) Appl. No.: 18/366,187

(22) Filed: Aug. 7, 2023

Related U.S. Application Data
(60) Provisional application No. 63/395,992, filed on Aug. 8, 2022.

(51) Int. Cl. C07D 401/04; C07D 401/14; C07D 413/14

(52) U.S. Cl. CPC C07D 401/04; C07D 401/14; C07D 413/14

(57) ABSTRACT

The present disclosure provides heterocyclic amide and urea compounds and compositions thereof useful for inhibiting JAK2.

### RELATED APPLICATIONS

This application claims priority to and benefit of U.S. Appl. No. 63/395,992, filed on Aug. 8, 2022, the entire contents of which are hereby incorporated by reference.

### BACKGROUND

Janus kinase 2 (JAK2) is a non-receptor tyrosine kinase involved in the JAK-STAT signaling pathway, which plays a role in cell processes such as immunity, cell division, and cell death. Dysfunction of the JAK-STAT pathway is implicated in various diseases, including cancer and other proliferative diseases, as well as diseases of the immune system. For example, essentially all BCR-ABL1-negative myeloproliferative neoplasms are associated with mutations that activate JAK2. In particular, JAK2^{V617F} is the most prevalent mutation in myeloproliferative neoplasms, occurring in approx. 70% of all patients, and in up to 95% of patients with polycythemia vera. (Vainchenker, W., Kralovics, R. Blood 2017, 129(6):667-79). Even less common mutations, such as in MPL and CALR, have been shown to effect activation of JAK2, thereby initiating and/or driving disease progression. (Vainchenker, W. et al., F1000Research 2018, 7(F1000 Faculty Rev):82). Furthermore, polymorphisms in JAK2 have been linked to various autoimmune diseases and inflammatory conditions, such as psoriasis and inflammatory bowel disease. (O'Shea, J. J. et al., Ann. Rheum. Dis. 2013 Apr, 72:ii11-iil15). Increased signaling through JAK2, as well as other members of the JAK family, is also associated with atopic dermatitis. (Rodrigues, M. A. and Torres, T. J. Derm. Treat. 2019, 31(1):33-40).

Inhibitors of JAKs (e.g., JAK2) are classified based on their binding mode. All currently approved JAK inhibitors are Type I inhibitors, which are those that bind the ATP-binding site in the active conformation of the kinase domain, thereby blocking catalysis (Vainchenker, W. et al.). However, increased phosphorylation of the JAK2 activation loop is observed with Type I inhibitors and may lead to acquired resistance in certain patients (Meyer S. C., Levine, R. L. Clin. Cancer Res. 2014, 20(8):2051-9). Type II inhibitors, on the other hand, bind the ATP-binding site of the kinase domain in the inactive conformation and, therefore, may avoid hyperphosphorylation observed with Type I inhibitors (Wu, S. C. et al. Cancer Cell 2015 Jul. 13, 28(1):29-41).

### SUMMARY

The present disclosure provides compounds useful for inhibiting JAK2. In some embodiments, provided compounds are useful for, among other things, treating and/or preventing diseases, disorders, or conditions associated with JAK2.

In some embodiments, the present disclosure provides a compound of Formula A:

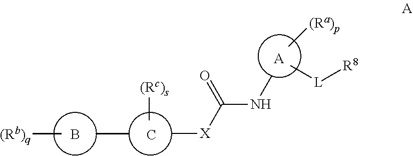

or a pharmaceutically acceptable salt thereof, wherein Ring A, Ring B, Ring C, L, p, q, s, R^{a}, R^{b}, R^{c}, R⁸, and X are as defined herein.

In some embodiments, the present disclosure provides a compound of Formula I:

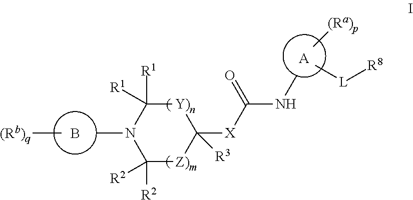

or a pharmaceutically acceptable salt thereof, wherein Ring A, Ring B, L, m, n, p, q, R^{a}, R^{b}, R¹, R², R³, R⁸, X, Y, and Z are as defined herein.

### DETAILED DESCRIPTION

### Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some case, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

Aliphatic: The term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that has a single point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment to the rest of the molecule.

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl. The term “alkylene,” as used herein, alone or in combination, refers to a bivalent, saturated, optionally substituted straight or branched hydrocarbon, such as methylene (—CH₂—).

Carbocyclyl: The terms “carbocyclyl,” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₅-C₁₀ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: The term “aryl” refers to monocyclic and bicyclic ring systems having a total of six to fourteen ring members (e.g., C₆-14), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but not limited to, phenyl, naphthyl, anthracyl and the like, which may bear one or more substituents. Unless otherwise specified, “aryl” groups are hydrocarbons.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 6- to 10-membered bicyclic heterocyclic moiety or a 10- to 16-membered polycyclic (i.e., comprising three or more rings) moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic or polycyclic heterocyclic ring can also be a spirocyclic ring system (e.g., 6- to 11-membered spirocyclic bicyclic heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

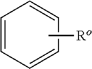

refers to at least

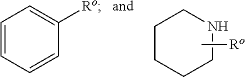

refers to at least

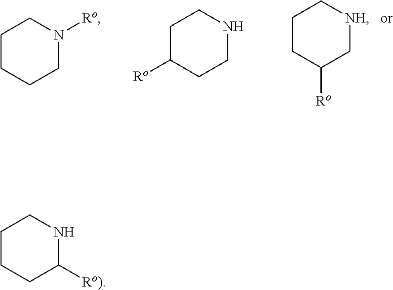

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)(NH)R^{o}; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

### Provided Compounds

In some embodiments, the present disclosure provides a compound of Formula A:

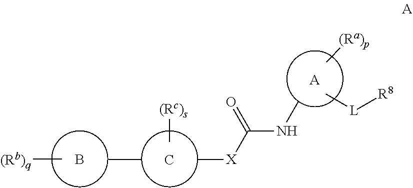

or a pharmaceutically acceptable salt thereof, wherein:
X is —C(R⁶)₂—, —N(R⁷)—, or —O—;
each R⁶ is independently hydrogen or optionally substituted C₁₋₆ aliphatic, or
   two R⁶ groups, together with the atom to which they are attached, combine to form a 3- to 8-membered saturated or partially unsaturated ring;
R⁷ is hydrogen or optionally substituted C₁₋₆ aliphatic;
Ring A is an optionally substituted group selected from phenyl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur;
Ring B is an optionally substituted group selected from 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, and 10- to 16-membered polycyclic heteroaryl having 1-5 heteroatoms independently selected from nitrogen, oxygen, and sulfur;
Ring C is an optionally substituted group selected from phenyl, 5- to 6-membered monocyclic heteroaryl having 1-4 heteroatoms independently selected from nitrogen, oxygen, and sulfur, 3- to 7-membered saturated or partially unsaturated monocyclic carbocyclyl, 5- to 8-membered saturated or partially unsaturated bicyclic carbocyclyl, 3-to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
L is a covalent bond or a bivalent C₁₋₃ straight or branched hydrocarbon chain;
R⁸ is hydrogen, halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur;
each R^{a} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
each R^{b} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
each R^{c} is independently halogen, —CN, —OR, or optionally substituted C₁₋₆ aliphatic, and/or
   two R^{c} groups and/or a R^{c} and R⁶ group and/or a R^{c} and R⁷ group, together with the atom(s) to which they are attached, combine to form an optionally substituted 3- to 8-membered saturated or partially unsaturated ring, and/or
   two R^{c} groups, together with the atom to which they are attached, combine to form an oxo;
p is 0, 1, 2, 3, 4, or 5, as valency permits;
q is 0, 1, 2, 3, 4, or 5, as valency permits;
s is 0, 1, 2, 3, 4, or 5, as valency permits;
each R is independently hydrogen or an optionally substituted group selected from C₁₋₆ aliphatic, C₃₋₇ cycloaliphatic, 3- to 7-membered saturated or partially unsaturated monocyclic heterocyclyl having 1-3 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or two R, when attached to the same nitrogen atom, are taken together to form an optionally substituted 3- to 7-membered saturated or partially unsaturated ring having 0-1 additional heteroatoms independently selected from nitrogen, oxygen, and sulfur; and
each R′ is independently an optionally substituted group selected from C₁₋₆ aliphatic and C₃₋₇ cycloaliphatic.

In some embodiments, the present disclosure provides a compound of Formula B:

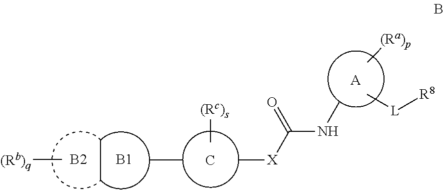

or a pharmaceutically acceptable salt thereof, wherein Ring A, Ring C, L, p, q, s, R^{a}, R^{b}, R^{c}, and X are as defined above for Formula A and described in classes and subclasses herein, both singly and in combination; and
Ring B1 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur,
wherein at least one of Ring B1 and Ring B2 is aromatic; and
wherein at least one of Ring B1 and Ring B2 contains a heteroatom.

In some embodiments, the present disclosure provides a compound of Formula C:

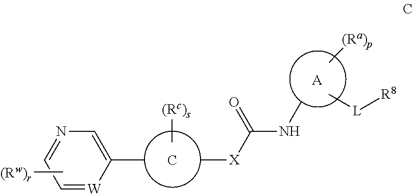

or a pharmaceutically acceptable salt thereof, wherein Ring A, Ring C, L, p, s, R^{a}, R^{b}, R^{c}, R′, R, R′, and X are as defined above for Formula A and described in classes and subclasses herein, both singly and in combination; and
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
   two R^{w} groups, together with the atoms to which they are attached, combine to form a 5-to 6-membered partially unsaturated or aromatic ring substituted with 0-4 R^{b} groups; and
r is 0, 1, 2, or 3.

In some embodiments, the present disclosure provides a compound of Formula D:

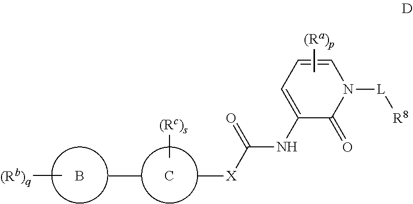

or a pharmaceutically acceptable salt thereof, wherein Ring B, Ring C, L, p, q, s, R^{a}, R^{b}, R^{c}, R⁸, and X are as defined above for Formula A and described in classes and subclasses herein, both singly and in combination.

In some embodiments, the present disclosure provides a compound of Formula E:

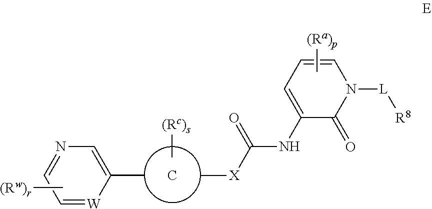

or a pharmaceutically acceptable salt thereof, wherein Ring C, L, p, s, R^{a}, R^{c}, R′, and X are as defined above for Formula A and described in classes and subclasses herein, both singly and in combination; and
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
   two R^{w} groups, together with the atoms to which they are attached, combine to form a 5-to 6-membered partially unsaturated or aromatic ring substituted with 0-4 R^{b} groups; and
r is 0, 1, 2, or 3.

In some embodiments, the present disclosure provides a compound of Formula A, wherein:
X is —C(R⁶)₂—, —N(R⁷)—, or —O—;
each R⁶ is hydrogen, or
   two R⁶ groups, together with the atom to which they are attached, combine to form a 3-membered carbocyclic ring;
R⁷ is C₁₋₆ alkyl;
Ring A is selected from phenyl and 5- to 6-membered monocyclic heteroaryl having 1-4 heteroatoms independently selected from nitrogen, oxygen, and sulfur;
Ring B is selected from 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; Ring C is an optionally substituted group selected from phenyl, 6-membered saturated or partially unsaturated monocyclic carbocyclyl, 5-membered saturated or partially unsaturated bicyclic carbocyclyl, 5- to 7-membered saturated or partially unsaturated monocyclic heterocyclyl having 1-2 heteroatoms independently selected from nitrogen, oxygen, and sulfur, and 7- to 8-membered saturated or partially unsaturated bicyclic heterocyclyl having 1-2 heteroatoms independently selected from nitrogen, oxygen, and sulfur;
L is a covalent bond or —CH₂—;
R⁸ is optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
each R^{a} is independently optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
each R^{b} is independently halogen, —CN, —OR, —N(R)₂, —C(O)N(R)₂, —N(R)C(O)R′, —N(R)C(O)N(R)₂, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
each R^{c} is independently halogen, —OR, or optionally substituted C₁₋₆ alkyl, and/or
   two R^{c} groups and/or a R^{c} and R⁷ group, together with the atom(s) to which they are attached, combine to form an optionally substituted 3- to 8-membered saturated or partially unsaturated ring;
p is 0, 1, 2, or 3;
q is 0, 1, 2, or 3;
s is 0, 1, 2, or 3;
each R is independently hydrogen or an optionally substituted group selected from C₁₋₆ alkyl, C₃₋₇ cycloalkyl, and 3- to 7-membered saturated monocyclic heterocyclyl having 1-3 heteroatoms independently selected from nitrogen, oxygen, and sulfur; and
each R′ is independently an optionally substituted group selected from C₁₋₆ alkyl and C₃₋₇ cycloalkyl.

In some embodiments, the present disclosure provides a compound of Formula A, wherein Ring A, Ring B, L, R^{a}, R^{b}, R^{c}, R⁸, q, p, s, and X are as defined above for Formula A and described in classes and subclasses herein, both singly and in combination; and wherein Ring C comprises a N atom as the point of attachment to Ring B and is an optionally substituted group selected from 3- to 7-membered saturated or partially unsaturated monocyclic heterocyclyl having 1-3 heteroatoms independently selected from nitrogen, oxygen, and sulfur and 6- to 10-membered saturated or partially unsaturated bicyclic heterocyclyl having 1-4 heteroatoms independently selected from nitrogen, oxygen, and sulfur.

In some embodiments, the present disclosure provides a compound of Formula I:

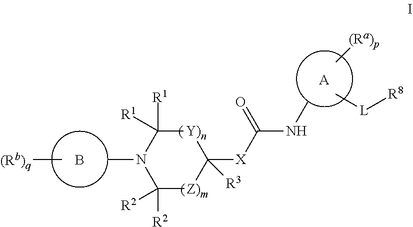

or a pharmaceutically acceptable salt thereof, wherein:
X is —C(R⁶)₂—, —N(R⁷)—, or —O—;
each Y is —C(R⁴)₂—;
each Z is —C(R⁵)₂—;
n is 0, 1, or 2;
m is 0, 1, or 2,
   provided that at least one of n or m is 1 or 2;
each R¹, R², R³, R⁴, and R⁵ is independently hydrogen, halogen, —CN, —OR, or optionally substituted C₁₋₆ aliphatic, and/or
   two R¹ groups and/or two R² groups and/or two R⁴ groups and/or two R⁵ groups, together with the atom(s) to which they are attached, combine to form a 3- to 8-membered saturated or partially unsaturated ring, and/or
   two R¹ groups and/or two R² groups and/or two R⁴ groups and/or two R⁵ groups, together with the atom to which they are attached, combine to form an oxo, and/or
   a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group, together with the atoms to which they are attached, combine to form an optionally substituted 3- to 8-membered saturated or partially unsaturated ring;
each R⁶ is independently hydrogen or optionally substituted C₁₋₆ aliphatic, or two R⁶ groups, together with the atom to which they are attached, combine to form a 3- to 8-membered saturated or partially unsaturated ring;
R⁷ is hydrogen or optionally substituted C₁₋₆ aliphatic;
Ring A is an optionally substituted group selected from phenyl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur;
Ring B is an optionally substituted group selected from 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, and 10- to 16-membered polycyclic heteroaryl having 1-5 heteroatoms independently selected from nitrogen, oxygen, and sulfur;
L is a covalent bond or a bivalent C₁₋₃ straight or branched hydrocarbon chain;
R⁸ is hydrogen, halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur;
each R^{a} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
each R^{b} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
p is 0, 1, 2, 3, 4, or 5, as valency permits;
q is 0, 1, 2, 3, 4, or 5, as valency permits;
each R is independently hydrogen or an optionally substituted group selected from C₁₋₆ aliphatic, C₃₋₇ cycloaliphatic, 3- to 7-membered saturated or partially unsaturated monocyclic heterocyclyl having 1-3 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
   two R, when attached to the same nitrogen atom, are taken together to form an optionally substituted 3- to 7-membered saturated or partially unsaturated ring having 0-1 additional heteroatoms independently selected from nitrogen, oxygen, and sulfur; and
each R′ is independently an optionally substituted group selected from C₁₋₆ aliphatic and C₃₋₇ cycloaliphatic.

In some embodiments, the present disclosure provides a compound of Formula I, wherein:
X is —C(R⁶)₂— or —N(R⁷)—;
each Y is —C(R⁴)₂—;
each Z is —C(R⁵)₂—;
n is 0, 1, or 2;
m is 0, 1, or 2,
   provided that at least one of n or m is 1 or 2;
each R¹, R², R³, R⁴, and R⁵ is independently hydrogen, halogen, —CN, or optionally substituted C₁₋₆ aliphatic, and/or
   two R¹ groups and/or two R² groups and/or two R⁴ groups and/or two R⁵ groups, together with the atom(s) to which they are attached, combine to form a 3- to 8-membered saturated or partially unsaturated ring, and/or
   two R¹ groups and/or two R² groups and/or two R⁴ groups and/or two R⁵ groups, together with the atom to which they are attached, combine to form an oxo, and/or
   a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group, together with the atoms to which they are attached, combine to form a 3- to 8-membered saturated or partially unsaturated ring;
each R⁶ is independently hydrogen or optionally substituted C₁₋₆ aliphatic, or two R⁶ groups, together with the atom to which they are attached, combine to form a 3- to 8-membered saturated or partially unsaturated ring;
R⁷ is hydrogen or optionally substituted C₁₋₆ aliphatic;
Ring A is an optionally substituted group selected from phenyl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur;
Ring B is an optionally substituted group selected from 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, or 10- to 16-membered polycyclic heteroaryl having 1-5 heteroatoms independently selected from nitrogen, oxygen, and sulfur;
L is a covalent bond or a bivalent C₁₋₃ straight or branched hydrocarbon chain;
R⁸ is hydrogen, halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur;
each R^{a} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
each R^{b} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
p is 0, 1, 2, 3, 4, or 5, as valency permits;
q is 0, 1, 2, 3, 4, or 5, as valency permits;
each R is independently hydrogen or an optionally substituted group selected from C₁₋₆ aliphatic, C₃₋₇ cycloaliphatic, 3- to 7-membered saturated or partially unsaturated monocyclic heterocyclyl having 1-3 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
   two R, when attached to the same nitrogen atom, are taken together to form an optionally substituted 3- to 7-membered saturated or partially unsaturated ring having 0-1 additional heteroatoms independently selected from nitrogen, oxygen, and sulfur; and
each R′ is independently an optionally substituted group selected from C₁₋₆ aliphatic and C₃₋₇ cycloaliphatic.

In some embodiments, the present disclosure provides a compound of Formula I, wherein:
X is —C(R⁶)₂—, —N(R⁷)—, or —O—;
each Y is —C(R⁴)₂—;
each Z is —C(R⁵)₂—;
n is 1 or 2;
m is 1;
each R¹, R², R³, R⁴, and R⁵ is independently hydrogen, halogen, —OR, or optionally substituted C₁₋₆ alkyl, and/or
   a R¹ and R⁵ group and/or a R³ and R⁷ group and/or a R⁴ and R⁵ group and/or a R⁴ and R⁷ group, together with the atoms to which they are attached, combine to form an optionally substituted 3- to 8-membered saturated or partially unsaturated ring;
each R⁶ is independently hydrogen, or two R⁶ groups, together with the atom to which they are attached, combine to form a 3-membered carbocyclic ring;
R⁷ is C₁₋₆ alkyl;
Ring A is selected from phenyl and 5- to 6-membered monocyclic heteroaryl having 1-4 heteroatoms independently selected from nitrogen, oxygen, and sulfur;
Ring B is selected from 5- to 6-membered monocyclic heteroaryl having 1-4 heteroatoms independently selected from nitrogen, oxygen, and sulfur and 8- to 10-membered bicyclic heteroaryl having 1-4 heteroatoms independently selected from nitrogen, oxygen, and sulfur;
L is a covalent bond or —CH₂—;
R⁸ is optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
each R^{a} is independently optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
each R^{b} is independently halogen, —CN, —OR, —N(R)₂, —C(O)N(R)₂, —N(R)C(O)R′, —N(R)C(O)N(R)₂, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur;
p is 0, 1, 2, or 3;
q is 0, 1, 2, or 3;
each R is independently hydrogen or an optionally substituted group selected from C₁₋₆ alkyl, C₃₋₇ cycloalkyl, and 3- to 7-membered saturated monocyclic heterocyclyl having 1-3 heteroatoms independently selected from nitrogen, oxygen, and sulfur; and
each R′ is independently an optionally substituted group selected from C₁₋₆ alkyl and C₃₋₇ cycloalkyl.

In some embodiments, the present disclosure provides a compound of Formula IA:

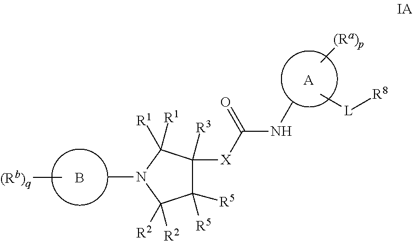

or a pharmaceutically acceptable salt thereof, wherein Ring A, Ring B, L, p, q, R^{a}, R^{b}, R¹, R², R³, R⁵, R⁸, and X are as defined above for Formula I and described in classes and subclasses herein, both singly and in combination.

In some embodiments, the present disclosure provides a compound of Formula IB:

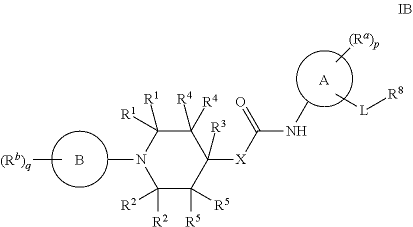

or a pharmaceutically acceptable salt thereof, wherein Ring A, Ring B, L, p, q, R^{a}, R^{b}, R¹, R², R³, R⁴, R⁵, R′, and X are as defined above for Formula I and described in classes and subclasses herein, both singly and in combination.

In some embodiments, the present disclosure provides a compound of Formula IC:

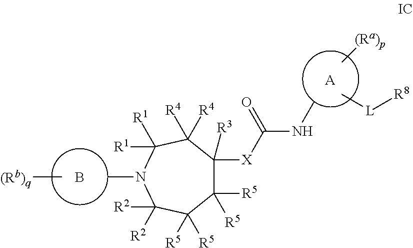

or a pharmaceutically acceptable salt thereof, wherein Ring A, Ring B, L, p, q, R^{a}, R^{b}, R¹, R², R³, R⁴, R⁵, R⁸, and X are as defined above for Formula I and described in classes and subclasses herein, both singly and in combination.

In some embodiments, the present disclosure provides a compound of Formula ID:

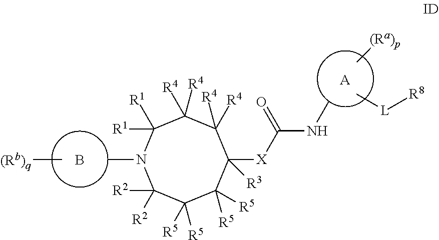

or a pharmaceutically acceptable salt thereof, wherein Ring A, Ring B, L, p, q, R^{a}, R^{b}, R¹, R², R³, R⁴, R⁵, R⁸, and X are as defined above for Formula I and described in classes and subclasses herein, both singly and in combination.

In some embodiments, the present disclosure provides a compound of Formula II:

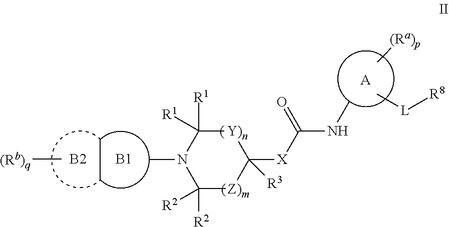

or a pharmaceutically acceptable salt thereof, wherein Ring A, L, m, n, p, q, R^{a}, R^{b}, R¹, R², R³, R⁸, X, Y, and Z are as defined above for Formula I and described in classes and subclasses herein, both singly and in combination; and
Ring B1 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur,
wherein at least one of Ring B1 and Ring B2 is aromatic; and
wherein at least one of Ring B1 and Ring B2 contains a heteroatom.

In some embodiments, the present disclosure provides a compound of Formula III:

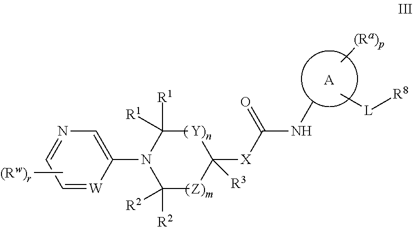

or a pharmaceutically acceptable salt thereof, wherein Ring A, L, m, n, p, R^{a}, R¹, R², R³, R′, X, Y, R, R′, and Z are as defined above for Formula I and described in classes and subclasses herein, both singly and in combination; and
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
   two R^{w} groups, together with the atoms to which they are attached, combine to form a 5-to 6-membered partially unsaturated or aromatic ring substituted with 0-4 R^{b} groups; and
r is 0, 1, 2, or 3.

In some embodiments, the present disclosure provides a compound of Formula IV:

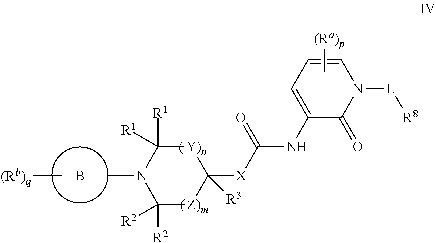

or a pharmaceutically acceptable salt thereof, wherein Ring B, L, m, n, p, q, R^{a}, R^{b}, R¹, R², R³, R⁸, X, Y, and Z are as defined above for Formula I and described in classes and subclasses herein, both singly and in combination.

In some embodiments, the present disclosure provides a compound of Formula V:

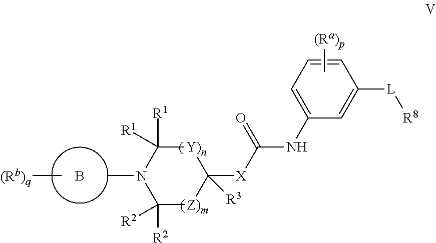

or a pharmaceutically acceptable salt thereof, wherein Ring B, L, m, n, p, q, R^{a}, R^{b}, R¹, R², R³, R⁸, X, Y, and Z are as defined above for Formula I and described in classes and subclasses herein, both singly and in combination.

In some embodiments, the present disclosure provides a compound of Formula VI:

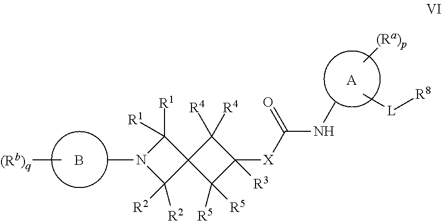

or a pharmaceutically acceptable salt thereof, wherein Ring A, Ring B, L, p, q, R^{a}, R^{b}, R′, and X are as defined above for Formula A1 and described in classes and subclasses herein, both singly and in combination; and wherein each R¹, R², R³, R⁴, and R⁵ is independently hydrogen, halogen, —CN, or optionally substituted C₁₋₆ aliphatic.

In some embodiments, the present disclosure provides a compound of Formula A, wherein Ring A, Ring B, L, R^{a}, R^{b}, R^{c}, R′, q, p, s, and X are as defined above for Formula A and described in classes and subclasses herein, both singly and in combination; and wherein Ring C comprises a carbon atom as the point of attachment to Ring B and is an optionally substituted group selected from phenyl, 3- to 7-membered saturated or partially unsaturated monocyclic carbocyclyl, 5- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur and 6- to 10-membered saturated or partially unsaturated bicyclic heterocyclyl having 1-4 heteroatoms independently selected from nitrogen, oxygen, and sulfur.

In some embodiments, the present disclosure provides a compound of Formula VII:

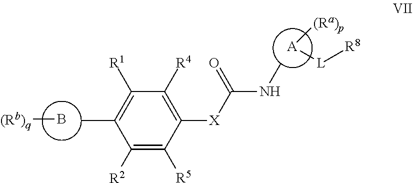

or a pharmaceutically acceptable salt thereof, wherein Ring A, Ring B, L, p, q, R^{a}, R^{b}, R′, and X are as defined above for Formula A2 and described in classes and subclasses herein, both singly and in combination; and wherein each R¹, R², R⁴, and R⁵ is independently hydrogen, halogen, —CN, or optionally substituted C₁₋₆ aliphatic.

In some embodiments, the present disclosure provides a compound of Formula VIII:

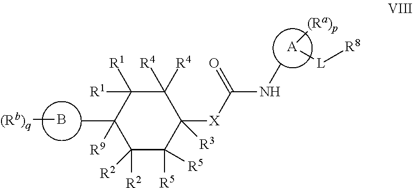

or a pharmaceutically acceptable salt thereof, wherein Ring A, Ring B, L, p, q, R^{a}, R^{b}, R′, and X are as defined above for Formula A2 and described in classes and subclasses herein, both singly and in combination; and wherein each R¹, R², R³, R⁴, R⁵, and R⁹ is independently hydrogen, halogen, —CN, or optionally substituted C₁₋₆ aliphatic.

In some embodiments, the present disclosure provides a compound of Formula IX:

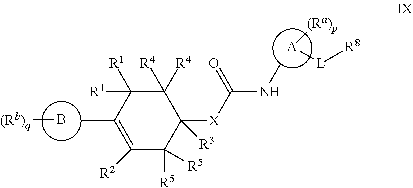

or a pharmaceutically acceptable salt thereof, wherein Ring A, Ring B, L, p, q, R^{a}, R^{b}, R⁸, and X are as defined above for Formula A2 and described in classes and subclasses herein, both singly and in combination; and wherein each R¹, R², R³, R⁴, and R⁵ is independently hydrogen, halogen, —CN, or optionally substituted C₁₋₆ aliphatic.

In some embodiments, the present disclosure provides a compound of Formula X:

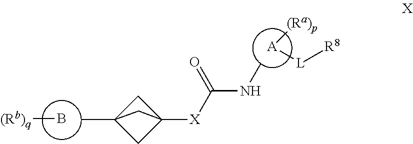

or a pharmaceutically acceptable salt thereof, wherein Ring A, Ring B, L, p, q, R^{a}, R^{b}, R′, and X are as defined above for Formula A2 and described in classes and subclasses herein, both singly and in combination.

In some embodiments, the present disclosure provides a compound of Formula XI:

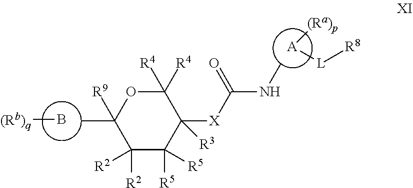

or a pharmaceutically acceptable salt thereof, wherein Ring A, Ring B, L, p, q, R^{a}, R^{b}, R′, and X are as defined above for Formula A2 and described in classes and subclasses herein, both singly and in combination; and
wherein each R², R³, R⁴, R⁵, and R⁹ is independently hydrogen, halogen, —CN, or optionally substituted C₁₋₆ aliphatic, and/or
two R² groups and/or two R³ groups and/or two R⁴ groups and/or two R⁵ groups, together with the atom to which they are attached, combine to form a 3- to 8-membered saturated or partially unsaturated ring, and/or
a R⁹ and R² group and/or a R⁹ and R³ group and/or a R⁹ and R⁴ group and/or a R⁹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group, together with the atoms to which they are attached, combine to form an optionally substituted 3- to 8-membered saturated or partially unsaturated ring.

In some embodiments of any of Formulae A, B, C, D, E, I, IA, IB, IC, ID, II, III, IV, V, VI, VII, VIII, IX, X, and XI, X is —C(R⁶)₂— or —N(R⁷)—. In some embodiments, X is —C(R⁶)₂—. In some embodiments, X is —N(R⁷)—. In some embodiments, X is —O—.

In some embodiments of any of Formulae A, B, C, D, and E, Ring C is selected from phenyl, 3- to 7-membered saturated or partially unsaturated monocyclic carbocyclyl, 5- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur. In some embodiments, Ring C is selected from phenyl, 3- to 7-membered saturated or partially unsaturated monocyclic carbocyclyl, and 5- to 8-membered saturated or partially unsaturated bicyclic carbocyclyl. In some embodiments, Ring C is selected 3- to 7-membered saturated or partially unsaturated monocyclic carbocyclyl and 5- to 8-membered saturated or partially unsaturated bicyclic carbocyclyl. In some embodiments, Ring C is selected from 3- to 7-membered saturated or partially unsaturated monocyclic heterocyclyl having 1-3 heteroatoms independently selected from nitrogen, oxygen, and sulfur and 6- to 10-membered saturated or partially unsaturated bicyclic heterocyclyl having 1-4 heteroatoms independently selected from nitrogen, oxygen, and sulfur.

In some embodiments, Ring C comprises a N atom as the point of attachment to Ring B and is selected from 3- to 7-membered saturated or partially unsaturated monocyclic heterocyclyl having 1-3 heteroatoms independently selected from nitrogen, oxygen, and sulfur and 6- to 10-membered saturated or partially unsaturated bicyclic heterocyclyl having 1-4 heteroatoms independently selected from nitrogen, oxygen, and sulfur. In some such embodiments, Ring C is

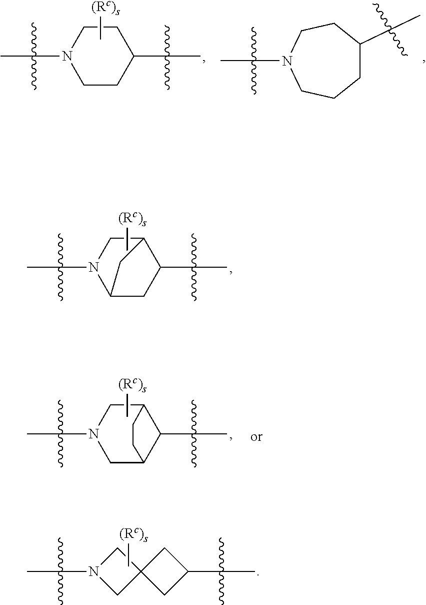

In some embodiments, Ring C comprises a C atom as the point of attachment to Ring B and is selected from phenyl, 3- to 7-membered saturated or partially unsaturated monocyclic carbocyclyl, 5- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur. In some embodiments, Ring C comprises a C atom as the point of attachment to Ring B and is selected from phenyl, 3- to 7-membered saturated or partially unsaturated monocyclic carbocyclyl, and 5- to 8-membered saturated or partially unsaturated bicyclic carbocyclyl. In some embodiments, Ring C comprises a C atom as the point of attachment to Ring B and is selected from 3- to 7-membered saturated or partially unsaturated monocyclic heterocyclyl having 1-3 heteroatoms independently selected from nitrogen, oxygen, and sulfur and 6- to 10-membered saturated or partially unsaturated bicyclic heterocyclyl having 1-4 heteroatoms independently selected from nitrogen, oxygen, and sulfur. In some such embodiments, Ring C is

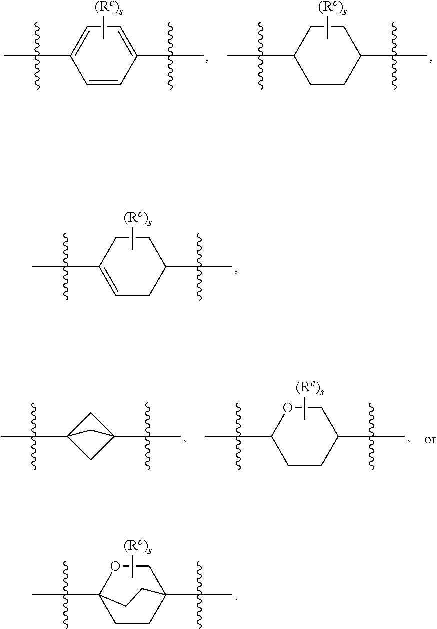

In some embodiments, Ring C is phenyl. In some embodiments, Ring C is

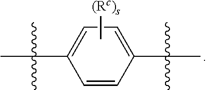

In some embodiments, Ring C is 3- to 7-membered saturated or partially unsaturated monocyclic carbocyclyl. In some embodiments, Ring C is 5- to 7-membered saturated or partially unsaturated monocyclic carbocyclyl. In some embodiments, Ring C is a 3-membered saturated or partially unsaturated monocyclic carbocyclyl. In some embodiments, Ring C is a 4-membered saturated or partially unsaturated monocyclic carbocyclyl. In some embodiments, Ring C is a 5-membered saturated or partially unsaturated monocyclic carbocyclyl. In some embodiments, Ring C is a 6-membered saturated or partially unsaturated monocyclic carbocyclyl. In some embodiments, Ring C is a cyclohexane or cyclohexene ring. In some embodiments, Ring C is a 7-membered saturated or partially unsaturated monocyclic carbocyclyl. In some embodiments, Ring C is

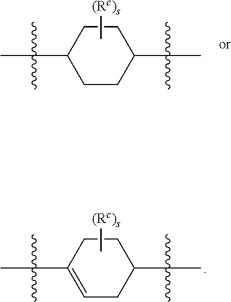

In some embodiments, Ring C is a 5- to 8-membered saturated or partially unsaturated bicyclic carbocyclyl. In some embodiments, Ring C is a 5- to 6-membered saturated bicyclic carbocyclyl. In some embodiments, Ring C is a 5-membered saturated bicyclic carbocyclyl. In some embodiments, Ring C is a 6-membered saturated or partially unsaturated bicyclic carbocyclyl. In some embodiments, Ring C is a 7-membered saturated or partially unsaturated bicyclic carbocyclyl. In some embodiments, Ring C is a 8-membered saturated or partially unsaturated bicyclic carbocyclyl. In some embodiments, Ring C is

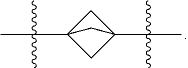 [SMILES: *C12CC(*)(C1)C2]

In some embodiments, Ring C is a 3- to 7-membered saturated or partially unsaturated monocyclic heterocyclyl having 1-3 heteroatoms independently selected from nitrogen, oxygen, and sulfur. In some embodiments, Ring C is a 5- to 7-membered saturated or partially unsaturated monocyclic heterocyclyl having 1-3 heteroatoms independently selected from nitrogen, oxygen, and sulfur. In some embodiments, Ring C is a 6- to 7-membered saturated monocyclic heterocyclyl having 1-3 heteroatoms independently selected from nitrogen, oxygen, and sulfur. In some embodiments, Ring C is a 3-membered saturated or partially unsaturated monocyclic heterocyclyl having 1 heteroatom independently selected from nitrogen, oxygen, and sulfur. In some embodiments, Ring C is a 4-membered saturated or partially unsaturated monocyclic heterocyclyl having 1 heteroatom independently selected from nitrogen, oxygen, and sulfur. In some embodiments, Ring C is a 5-membered saturated or partially unsaturated monocyclic heterocyclyl having 1-2 heteroatoms independently selected from nitrogen, oxygen, and sulfur. In some embodiments, Ring C is a 6-membered saturated or partially unsaturated monocyclic heterocyclyl having 1-2 heteroatoms independently selected from nitrogen, oxygen, and sulfur. In some embodiments, Ring C is a 6-membered saturated monocyclic heterocyclyl having 1-2 heteroatoms independently selected from nitrogen, oxygen, and sulfur. In some embodiments, Ring C is a piperidine ring. In some embodiments, Ring C is a 7-membered saturated or partially unsaturated monocyclic heterocyclyl having 1-3 heteroatoms independently selected from nitrogen, oxygen, and sulfur. In some embodiments, Ring C is a 7-membered saturated monocyclic heterocyclyl having 1-3 heteroatoms independently selected from nitrogen, oxygen, and sulfur. In some embodiments, Ring C is an azepane ring. In some embodiments, Ring C is

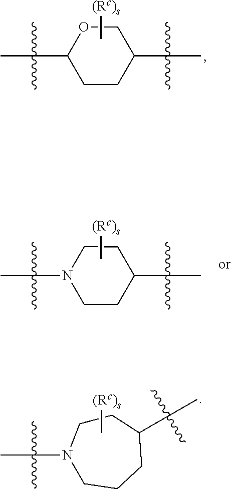

In some embodiments, Ring C is a 6- to 10-membered saturated or partially unsaturated bicyclic heterocyclyl having 1-4 heteroatoms independently selected from nitrogen, oxygen, and sulfur. In some embodiments, Ring C is a 7- to 8-membered saturated or partially unsaturated bicyclic heterocyclyl having 1-2 heteroatoms independently selected from nitrogen, oxygen, and sulfur. In some embodiments, Ring C is a 7- to 8-membered saturated bicyclic heterocyclyl having 1-2 heteroatoms independently selected from nitrogen, oxygen, and sulfur. In some embodiments, Ring C is a 6-membered saturated or partially unsaturated bicyclic heterocyclyl having 1-2 heteroatoms independently selected from nitrogen, oxygen, and sulfur. In some embodiments, Ring C is a 7-membered saturated or partially unsaturated bicyclic heterocyclyl having 1-2 heteroatoms independently selected from nitrogen, oxygen, and sulfur. In some embodiments, Ring C is a 8-membered saturated or partially unsaturated bicyclic heterocyclyl having 1-2 heteroatoms independently selected from nitrogen, oxygen, and sulfur. In some embodiments, Ring C is a 9-membered saturated or partially unsaturated bicyclic heterocyclyl having 1-3 heteroatoms independently selected from nitrogen, oxygen, and sulfur. In some embodiments, Ring C is a 10-membered saturated or partially unsaturated bicyclic heterocyclyl having 1-3 heteroatoms independently selected from nitrogen, oxygen, and sulfur. In some embodiments, Ring C is

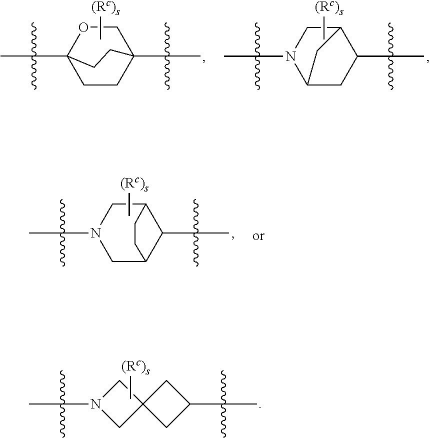

In some embodiments of any of Formulae A, B, C, D, and E, each R^{c} is independently halogen, —CN, —OR, or optionally substituted C₁₋₆ aliphatic, and/or two R^{c} groups and/or a R^{c} and R⁶ group and/or a R^{c} and R⁷ group, together with the atom(s) to which they are attached, combine to form an optionally substituted 3- to 8-membered saturated or partially unsaturated ring. In some embodiments, each R^{c} is independently halogen, —CN, —OR, or optionally substituted C₁₋₆ aliphatic, and/or two R^{c} groups and/or a R^{c} and R⁶ group and/or a R^{c} and R⁷ group, together with the atom(s) to which they are attached, combine to form an optionally substituted 3- to 6-membered saturated or partially unsaturated ring.

In some embodiments, each R^{c} is independently halogen, —CN, —OR, or optionally substituted C₁₋₆ aliphatic. In some embodiments, each R^{c} is independently halogen, —CN, —OR, or optionally substituted C₁₋₆ alkyl. In some embodiments, each R^{c} is halogen. In some embodiments, each R^{c} is —CN. In some embodiments, each R^{c} is —OR (e.g., —OCH₃ or —OCHF₂). In some embodiments, each R^{c} is optionally substituted C₁₋₆ alkyl (e.g., methyl).

In some embodiments, two R^{c} groups, together with the atom(s) to which they are attached, combine to form an optionally substituted 3- to 8-membered saturated or partially unsaturated ring. In some embodiments, two R^{c} groups, together with the atom(s) to which they are attached, combine to form an optionally substituted 3- to 6-membered saturated or partially unsaturated ring. In some embodiments, two R^{c} groups, together with the atom(s) to which they are attached, combine to form a C₃-C₆ cycloalkyl. In some embodiments, two R groups, together with the atom(s) to which they are attached, combine to form a 3- to 6-membered saturated or partially unsaturated heterocyclyl having 1-2 heteroatoms independently selected from nitrogen, oxygen, and sulfur. In some embodiments, two R^{c} groups, together with the atom(s) to which they are attached, combine to form a 3- to 6-membered saturated heterocyclyl having 1-2 heteroatoms independently selected from nitrogen, oxygen, and sulfur.

In some embodiments, a R^{c} and R⁶ group, together with the atoms to which they are attached, combine to form an optionally substituted 3- to 8-membered saturated or partially unsaturated ring. In some embodiments, a R and R⁶ group, together with the atoms to which they are attached, combine to form an optionally substituted 3- to 6-membered saturated or partially unsaturated ring. In some embodiments, a R^{c} and R⁶ group, together with the atoms to which they are attached, combine to form a C₃-C₆ cycloalkyl. In some embodiments, a R^{c} and R⁶ group, together with the atoms to which they are attached, combine to form a 3- to 6-membered saturated or partially unsaturated heterocyclyl having 1-2 heteroatoms independently selected from nitrogen, oxygen, and sulfur. In some embodiments, a R^{c} and R⁶ group, together with the atoms to which they are attached, combine to form a 3- to 6-membered saturated heterocyclyl having 1-2 heteroatoms independently selected from nitrogen, oxygen, and sulfur.

In some embodiments, a R^{c} and R⁷ group, together with the atoms to which they are attached, combine to form an optionally substituted 3- to 8-membered saturated or partially unsaturated ring. In some embodiments, a R^{c} and R⁷ group, together with the atoms to which they are attached, combine to form an optionally substituted 3- to 6-membered saturated or partially unsaturated ring. In some embodiments, a R and R⁷ group, together with the atoms to which they are attached, combine to form a 3- to 6-membered saturated or partially unsaturated heterocyclyl having 1-2 heteroatoms independently selected from nitrogen, oxygen, and sulfur. In some embodiments, a R^{c} and R⁷ group, together with the atoms to which they are attached, combine to form a 3- to 6-membered saturated heterocyclyl having 1-2 heteroatoms independently selected from nitrogen, oxygen, and sulfur.

In some embodiments, only one pair combines to form an optionally substituted 3- to 8-membered saturated or partially unsaturated ring, wherein the pair is selected from two R^{o} groups, a R¹ and R⁶ group, and a R¹ and R⁷ group.

In some embodiments, two R^{o} groups on the same carbon atom, together with the carbon atom to which they are attached, combine to form an oxo. In some embodiments, only one pair of R′ groups combines to form an oxo.

In some embodiments of any of Formulae A, B, C, D, and E, s is 0, 1, 2, 3, or 4. In some embodiments, s is 0, 1, 2, or 3. In some embodiments, s is 0, 1, or 2. In some embodiments, s is 0 or 1. In some embodiments, s is 1, 2, 3, 4, or 5. In some embodiments, s is 1, 2, or 3. In some embodiments, s is 1 or 2. In some embodiments, s is 0. In some embodiments, s is 1. In some embodiments, s is 2. In some embodiments, s is 3. In some embodiments, s is 4. In some embodiments, s is 5.

In some embodiments of any of Formulae I, II, III, IV, and V, n is 1 or 2. In some embodiments, n is 0 or 1. In some embodiments, n is 0. In some embodiments, n is 1. In some embodiments, n is 2.

In some embodiments of any of Formulae I, II, III, IV, and V, m is 1 or 2. In some embodiments, m is 0 or 1. In some embodiments, m is 0. In some embodiments, m is 1. In some embodiments, m is 2.

In some embodiments of any of Formulae I, II, III, IV, and V, n is 0 and m is 1. In some embodiments, n is 0 and m is 2. In some embodiments, n is 1 and m is 0. In some embodiments, n is 1 and m is 1. In some embodiments, n is 1 and m is 2. In some embodiments, n is 2 and m is 0. In some embodiments, n is 2 and m is 1. In some embodiments, n is 2 and m is 2.

In some embodiments of any of Formulae I, IA, IB, IC, ID, II, III, IV, V, VI, VII, and IX, each R¹, R², R³, R⁴, and R⁵ is independently hydrogen, halogen, —CN, —OR, or optionally substituted C₁₋₆ aliphatic. In some embodiments, each R¹, R², R³, R⁴, and R⁵ is independently hydrogen, halogen, —CN, or optionally substituted C₁₋₆ aliphatic. In some embodiments, each R¹, R², R³, R⁴, and R⁵ is independently hydrogen, halogen, —CN, —OR, or optionally substituted C₁₋₆ alkyl. In some embodiments, each R¹, R², R³, R⁴, and R⁵ is independently hydrogen or halogen. In some embodiments, each R¹, R², R³, R⁴, and R⁵ is hydrogen. In some embodiments, each R¹, R², R³, and R⁴ is hydrogen, and each R⁵ is independently hydrogen, halogen, —CN, —OR, or optionally substituted C₁₋₆ aliphatic. In some embodiments, each R¹, R², R³, and R⁴ is hydrogen, and each R⁵ is independently hydrogen, halogen, —CN, or optionally substituted C₁₋₆ aliphatic. In some embodiments, each R¹, R², R³, R⁴, and R⁵ is independently hydrogen, halogen, —CN, —O(C₁₋₆ alkyl) (e.g., —OCH₃), —O(C₁₋₆ haloalkyl) (e.g., —OCHF₂), or C₁₋₆ alkyl (e.g., methyl).

In some embodiments of any of Formulae VIII and XI, each R¹, R², R³, R⁴, R⁵, and R⁹ is independently hydrogen, halogen, —CN, —OR, or optionally substituted C₁₋₆ aliphatic. In some embodiments, each R¹, R², R³, R⁴, R⁵, and R⁹ is independently hydrogen, halogen, —CN, or optionally substituted C₁₋₆ aliphatic. In some embodiments, each R¹, R², R³, R⁴, R⁵, and R⁹ is independently hydrogen, halogen, —CN, —OR, or optionally substituted C₁₋₆ alkyl. In some embodiments, each R¹, R², R³, R⁴, R⁵, and R⁹ is independently hydrogen or halogen. In some embodiments, each R¹, R², R³, R⁴, R⁵, and R⁹ is hydrogen.

In some embodiments of any of Formulae I, IA, IB, IC, ID, II, III, IV, V, VI, VII, VIII, and IX, each R¹ is independently hydrogen, halogen, —CN, —OR, or optionally substituted C₁₋₆ aliphatic; or two R¹ groups, together with the atom to which they are attached, combine to form a 3- to 8-membered saturated or partially unsaturated ring; or two R¹ groups, together with the atom to which they are attached, combine to form an oxo; or a R¹ and R² group and/or a R¹ and R³ group and/or a R¹ and R⁴ group and/or a R¹ and R⁵ group, together with the atoms to which they are attached, combine to form an optionally substituted 3- to 8-membered saturated or partially unsaturated ring. In some embodiments, each R¹ is independently hydrogen, halogen, —CN, or optionally substituted C₁₋₆ aliphatic; or two R¹ groups, together with the atom to which they are attached, combine to form a 3- to 8-membered saturated or partially unsaturated ring; or two R¹ groups, together with the atom to which they are attached, combine to form an oxo; or a R¹ and R² group and/or a R¹ and R³ group and/or a R¹ and R⁴ group and/or a R¹ and R⁵ group, together with the atoms to which they are attached, combine to form a 3- to 8-membered saturated or partially unsaturated ring. In some embodiments, each R¹ is independently hydrogen, —CN, or optionally substituted C₁₋₆ aliphatic; or two R¹ groups, together with the atom to which they are attached, combine to form a 3- to 8-membered saturated or partially unsaturated ring; or two R¹ groups, together with the atom to which they are attached, combine to form an oxo; or a R¹ and R² group and/or a R¹ and R³ group and/or a R¹ and R⁴ group and/or a R¹ and R⁵ group, together with the atoms to which they are attached, combine to form a 3- to 8-membered saturated or partially unsaturated ring. In some embodiments, each R¹ is independently hydrogen, halogen, —CN, —OR, or optionally substituted C₁₋₆ aliphatic. In some embodiments, each R¹ is independently hydrogen, halogen, —CN, or optionally substituted C₁₋₆ aliphatic. In some embodiments, each R¹ group is hydrogen. In some embodiments, one R¹ is hydrogen, and the other R¹ is halogen, —CN, —OR, or optionally substituted C₁₋₆ aliphatic. In some embodiments, one R¹ is hydrogen, and the other R¹ is halogen, —CN, or optionally substituted C₁₋₆ aliphatic.

In some embodiments, R¹ is hydrogen. In some embodiments, R¹ is halogen (e.g., fluoro). In some embodiments, R¹ is —CN. In some embodiments, R¹ is —OR. In some embodiments, R¹ is optionally substituted C₁₋₆ aliphatic (e.g., optionally substituted C₁₋₆ alkyl). In some embodiments, R¹ is methyl.

In some embodiments, two R¹ groups, together with the atom to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, two R¹ groups, together with the atom to which they are attached, combine to form a 3- to 6-membered saturated or partially unsaturated carbocyclyl. In some embodiments, two R¹ groups, together with the atom to which they are attached, combine to form a C₃-C₆ cycloalkyl. In some embodiments, two R¹ groups, together with the atom to which they are attached, combine to form a 3- to 6-membered saturated or partially unsaturated heterocyclyl having 1-2 heteroatoms independently selected from nitrogen, oxygen, and sulfur. In some embodiments, two R¹ groups, together with the atom to which they are attached, combine to form a 3- to 6-membered saturated heterocyclyl having 1-2 heteroatoms independently selected from nitrogen, oxygen, and sulfur.

In some embodiments, two R¹ groups, together with the atom to which they are attached, combine to form an oxo.

In some embodiments of any of Formulae I, IA, IB, IC, ID, II, III, IV, V, VI, VII, VIII, IX, and XI, each R² is independently hydrogen, halogen, —CN, —OR, or optionally substituted C₁₋₆ aliphatic; or two R² groups, together with the atom to which they are attached, combine to form a 3- to 8-membered saturated or partially unsaturated ring; or two R² groups, together with the atom to which they are attached, combine to form an oxo; or a R¹ and R² group and/or a R⁹ and R² group and/or a R² and R³ group and/or a R² and R⁴ group and/or a R² and R⁵ group, together with the atoms to which they are attached, combine to form an optionally substituted 3- to 8-membered saturated or partially unsaturated ring. In some embodiments, each R² is independently hydrogen, halogen, —CN, or optionally substituted C₁₋₆ aliphatic; or two R² groups, together with the atom to which they are attached, combine to form a 3- to 8-membered saturated or partially unsaturated ring; or two R² groups, together with the atom to which they are attached, combine to form an oxo; or a R¹ and R² group and/or a R² and R³ group and/or a R² and R⁴ group and/or a R² and R⁵ group, together with the atoms to which they are attached, combine to form a 3- to 8-membered saturated or partially unsaturated ring. In some embodiments, each R² is independently hydrogen, —CN, or optionally substituted C₁₋₆ aliphatic; or two R² groups, together with the atom to which they are attached, combine to form a 3- to 8-membered saturated or partially unsaturated ring; or two R² groups, together with the atom to which they are attached, combine to form an oxo; or a R¹ and R² group and/or a R² and R³ group and/or a R² and R⁴ group and/or a R² and R⁵ group, together with the atoms to which they are attached, combine to form a 3- to 8-membered saturated or partially unsaturated ring. In some embodiments, each R² is independently hydrogen, halogen, —CN, —OR, or optionally substituted C₁₋₆ aliphatic. In some embodiments, each R² is independently hydrogen, halogen, —CN, or optionally substituted C₁₋₆ aliphatic. In some embodiments, each R² group is hydrogen. In some embodiments, one R² is hydrogen, and the other R² is halogen, —CN, —OR, or optionally substituted C₁₋₆ aliphatic. In some embodiments, one R² is hydrogen, and the other R² is halogen, —CN, or optionally substituted C₁₋₆ aliphatic.

In some embodiments, R² is hydrogen. In some embodiments, R² is halogen (e.g., fluoro). In some embodiments, R² is —CN. In some embodiments, R² is —OR. In some embodiments, R² is optionally substituted C₁₋₆ aliphatic (e.g., optionally substituted C₁₋₆ alkyl). In some embodiments, R² is methyl.

In some embodiments, two R² groups, together with the atom to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, two R² groups, together with the atom to which they are attached, combine to form a 3- to 6-membered saturated or partially unsaturated carbocyclyl. In some embodiments, two R² groups, together with the atom to which they are attached, combine to form a C₃-C₆ cycloalkyl. In some embodiments, two R² groups, together with the atom to which they are attached, combine to form a 3- to 6-membered saturated or partially unsaturated heterocyclyl having 1-2 heteroatoms independently selected from nitrogen, oxygen, and sulfur. In some embodiments, two R² groups, together with the atom to which they are attached, combine to form a 3- to 6-membered saturated heterocyclyl having 1-2 heteroatoms independently selected from nitrogen, oxygen, and sulfur.

In some embodiments, two R² groups, together with the atom to which they are attached, combine to form an oxo.

In some embodiments of any of Formulae I, IA, IB, IC, ID, II, III, IV, V, VI, VIII, IX, and XI, R³ is hydrogen, halogen, —CN, —OR, or optionally substituted C₁₋₆ aliphatic; or a R¹ and R³ group or a R⁹ and R³ group or a R² and R³ group or a R³ and R⁴ group or a R³ and R⁵ group or a R³ and R⁶ group or a R³ and R⁷ group, together with the atoms to which they are attached, combine to form an optionally substituted 3- to 8-membered saturated or partially unsaturated ring. In some embodiments, R³ is hydrogen, halogen, —CN, or optionally substituted C₁₋₆ aliphatic; or a R¹ and R³ group or a R² and R³ group or a R³ and R⁴ group or a R³ and R⁵ group or a R³ and R⁶ group or a R³ and R⁷ group, together with the atoms to which they are attached, combine to form a 3- to 8-membered saturated or partially unsaturated ring. In some embodiments, R³ is hydrogen, —CN, or optionally substituted C₁₋₆ aliphatic; or a R¹ and R³ group or a R² and R³ group or a R³ and R⁴ group or a R³ and R⁵ group or a R³ and R⁶ group or a R³ and R⁷ group, together with the atoms to which they are attached, combine to form a 3- to 8-membered saturated or partially unsaturated ring. In some embodiments, R³ is hydrogen, halogen, —CN, —OR, or optionally substituted C₁₋₆ aliphatic. In some embodiments, R³ is hydrogen, halogen, —CN, or optionally substituted C₁₋₆ aliphatic.

In some embodiments, R³ is hydrogen. In some embodiments, R³ is halogen (e.g., fluoro). In some embodiments, R³ is —CN. In some embodiments, R³ is —OR. In some embodiments, R³ is optionally substituted C₁₋₆ aliphatic (e.g., optionally substituted C₁₋₆ alkyl). In some embodiments, R³ is methyl.

In some embodiments of any of Formulae I, IB, IC, ID, II, III, IV, V, VI, VII, VIII, IX, and XI, each R⁴ is independently hydrogen, halogen, —CN, —OR, or optionally substituted C₁₋₆ aliphatic; and/or two R⁴ groups, together with the atom(s) to which they are attached, combine to form a 3- to 8-membered saturated or partially unsaturated ring; and/or two R⁴ groups, together with the atom to which they are attached, combine to form an oxo; and/or a R¹ and R⁴ group and/or a R⁹ and R⁴ group and/or a R² and R⁴ group and/or a R³ and R⁴ group and/or a R⁴ and R⁵ group and/or a R⁴ and R⁶ group and/or a R⁴ and R⁷ group, together with the atoms to which they are attached, combine to form an optionally substituted 3- to 8-membered saturated or partially unsaturated ring. In some embodiments, each R⁴ is independently hydrogen, halogen, —CN, or optionally substituted C₁₋₆ aliphatic; and/or two R⁴ groups, together with the atom(s) to which they are attached, combine to form a 3- to 8-membered saturated or partially unsaturated ring; and/or two R⁴ groups, together with the atom to which they are attached, combine to form an oxo; and/or a R¹ and R⁴ group and/or a R² and R⁴ group and/or a R³ and R⁴ group and/or a R⁴ and R⁵ group, together with the atoms to which they are attached, combine to form a 3- to 8-membered saturated or partially unsaturated ring. In some embodiments, each R⁴ is hydrogen, halogen (e.g., fluoro), —O(C₁₋₆ alkyl) (e.g., —OCH₃), —O(C₁₋₆ haloalkyl) (e.g., —OCHF₂), or C₁₋₆ alkyl (e.g., methyl). In some embodiments, each R⁴ is hydrogen, halogen (e.g., fluoro), or C₁₋₆ alkyl (e.g., methyl). In some embodiments, each R⁴ is hydrogen. In some embodiments, each R⁴ is halogen (e.g., fluoro). In some embodiments, each R⁴ is C₁₋₆ alkyl (e.g., methyl). In some embodiments, one R⁴ is hydrogen, and the other R⁴ is halogen (e.g., fluoro), —O(C₁₋₆ alkyl) (e.g., —OCH₃), —O(C₁₋₆ haloalkyl) (e.g., —OCHF₂), or C₁₋₆ alkyl (e.g., methyl).

In some embodiments, R⁴ is hydrogen. In some embodiments, R⁴ is halogen (e.g., fluoro). In some embodiments, R⁴ is —CN. In some embodiments, R⁴ is —OR. In some embodiments, R⁴ is —O(C₁₋₆ alkyl) (e.g., —OCH₃) or —O(C₁₋₆ haloalkyl) (e.g., —OCHF₂). In some embodiments, R⁴ is optionally substituted C₁₋₆ aliphatic (e.g., optionally substituted C₁₋₆ alkyl). In some embodiments, R⁴ is C₁₋₆ alkyl (e.g., methyl).

In some embodiments, two R⁴ groups, together with the atom(s) to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, two R⁴ groups, together with the atom(s) to which they are attached, combine to form a 3- to 6-membered saturated or partially unsaturated carbocyclyl. In some embodiments, two R⁴ groups, together with the atom(s) to which they are attached, combine to form a C₃-C₆ cycloalkyl. In some embodiments, two R⁴ groups, together with the atom(s) to which they are attached, combine to form a 3- to 6-membered saturated or partially unsaturated heterocyclyl having 1-2 heteroatoms independently selected from nitrogen, oxygen, and sulfur. In some embodiments, two R⁴ groups, together with the atom(s) to which they are attached, combine to form a 3- to 6-membered saturated heterocyclyl having 1-2 heteroatoms independently selected from nitrogen, oxygen, and sulfur.

In some embodiments, two R⁴ groups, together with the atom to which they are attached, combine to form an oxo.

In some embodiments of any of Formulae I, IA, IB, IC, ID, II, III, IV, V, VI, VII, VIII, IX, and XI, each R⁵ is independently hydrogen, halogen, —CN, —OR, or optionally substituted C₁₋₆ aliphatic; and/or two R⁵ groups, together with the atom(s) to which they are attached, combine to form a 3- to 8-membered saturated or partially unsaturated ring; and/or two R⁵ groups, together with the atom to which they are attached, combine to form an oxo; and/or a R¹ and R⁵ group and/or a R⁹ and R⁵ group and/or a R² and R⁵ group and/or a R³ and R⁵ group and/or a R⁴ and R⁵ group and/or a R⁵ and R⁶ group and/or a R⁵ and R⁷ group, together with the atoms to which they are attached, combine to form an optionally substituted 3- to 8-membered saturated or partially unsaturated ring. In some embodiments, each R⁵ is independently hydrogen, halogen, —CN, or optionally substituted C₁₋₆ aliphatic; and/or two R⁵ groups, together with the atom(s) to which they are attached, combine to form a 3- to 8-membered saturated or partially unsaturated ring; and/or two R⁵ groups, together with the atom to which they are attached, combine to form an oxo; and/or a R¹ and R⁵ group and/or a R² and R⁵ group and/or a R³ and R⁵ group and/or a R⁴ and R⁵ group, together with the atoms to which they are attached, combine to form a 3- to 8-membered saturated or partially unsaturated ring. In some embodiments, each R⁵ is hydrogen, halogen (e.g., fluoro), —O(C₁₋₆ alkyl) (e.g., —OCH₃), —O(C₁₋₆ haloalkyl) (e.g., —OCHF₂), or C₁₋₆ alkyl (e.g., methyl). In some embodiments, each R⁵ is hydrogen, halogen (e.g., fluoro), or C₁₋₆ alkyl (e.g., methyl). In some embodiments, each R⁵ is hydrogen. In some embodiments, each R⁵ is halogen (e.g., fluoro). In some embodiments, each R⁵ is C₁₋₆ alkyl (e.g., methyl). In some embodiments, one R⁵ is hydrogen, and the other R⁵ is halogen (e.g., fluoro), —O(C₁₋₆ alkyl) (e.g., —OCH₃), —O(C₁₋₆ haloalkyl) (e.g., —OCHF₂), or C₁₋₆ alkyl (e.g., methyl).

In some embodiments, R⁵ is hydrogen. In some embodiments, R⁵ is halogen (e.g., fluoro). In some embodiments, R⁵ is —CN. In some embodiments, R⁵ is —OR. In some embodiments, R⁵ is —O(C₁₋₆ alkyl) (e.g., —OCH₃) or —O(C₁₋₆ haloalkyl) (e.g., —OCHF₂). In some embodiments, R⁵ is optionally substituted C₁₋₆ aliphatic (e.g., optionally substituted C₁₋₆ alkyl). In some embodiments, R⁵ is C₁₋₆ alkyl (e.g., methyl).

In some embodiments, two R⁵ groups, together with the atom(s) to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, two R⁵ groups, together with the atom(s) to which they are attached, combine to form a 3- to 6-membered saturated or partially unsaturated carbocyclyl. In some embodiments, two R⁵ groups, together with the atom(s) to which they are attached, combine to form a C₃-C₆ cycloalkyl. In some embodiments, two R⁵ groups, together with the atom(s) to which they are attached, combine to form a 3- to 6-membered saturated or partially unsaturated heterocyclyl having 1-2 heteroatoms independently selected from nitrogen, oxygen, and sulfur. In some embodiments, two R⁵ groups, together with the atom(s) to which they are attached, combine to form a 3- to 6-membered saturated heterocyclyl having 1-2 heteroatoms independently selected from nitrogen, oxygen, and sulfur.

In some embodiments, two R⁵ groups, together with the atom to which they are attached, combine to form an oxo.

In some embodiments of any of Formulae VIII and XI, R⁹ is hydrogen, halogen, —CN, —OR, or optionally substituted C₁₋₆ aliphatic; or a R¹ and R⁹ group or a R² and R⁹ group or a R³ and R⁹ group or a R⁹ and R⁴ group or a R⁹ and R⁵ group, together with the atoms to which they are attached, combine to form an optionally substituted 3- to 8-membered saturated or partially unsaturated ring. In some embodiments, R⁹ is hydrogen, halogen, —CN, or optionally substituted C₁₋₆ aliphatic; or a R¹ and R⁹ group or a R² and R⁹ group or a R³ and R⁹ group or a R⁹ and R⁴ group or a R⁹ and R⁵ group, together with the atoms to which they are attached, combine to form a 3- to 8-membered saturated or partially unsaturated ring. In some embodiments, R⁹ is hydrogen, —CN, or optionally substituted C₁₋₆ aliphatic; or a R¹ and R⁹ group or a R² and R⁹ group or a R³ and R⁹ group or a R⁹ and R⁴ group or a R⁹ and R⁵ group, together with the atoms to which they are attached, combine to form a 3- to 8-membered saturated or partially unsaturated ring. In some embodiments, R⁹ is hydrogen, halogen, —CN, —OR, or optionally substituted C₁₋₆ aliphatic. In some embodiments, R⁹ is hydrogen, halogen, —CN, or optionally substituted C₁₋₆ aliphatic.

In some embodiments, R⁹ is hydrogen. In some embodiments, R⁹ is halogen (e.g., fluoro). In some embodiments, R⁹ is —CN. In some embodiments, R⁹ is —OR. In some embodiments, R⁹ is optionally substituted C₁₋₆ aliphatic (e.g., optionally substituted C₁₋₆ alkyl). In some embodiments, R⁹ is methyl.

In some embodiments of any of Formulae I, IA, IB, IC, ID, II, III, IV, V, VI, VII, VIII, IX, and XI, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group and/or a R¹ and R⁹ group and/or a R² and R⁹ group and/or a R³ and R⁹ group and/or a R⁹ and R⁴ group and/or a R⁹ and R⁵ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group and/or a R¹ and R⁹ group and/or a R² and R⁹ group and/or a R³ and R⁹ group and/or a R⁹ and R⁴ group and/or a R⁹ and R⁵ group, together with the atoms to which they are attached, combine to form an optionally substituted 3- to 6-membered saturated or partially unsaturated carbocyclyl (e.g., a C₃₋₆ cycloalkyl). In some embodiments, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group, together with the atoms to which they are attached, combine to form a 3- to 6-membered saturated or partially unsaturated carbocyclyl (e.g., a C₃₋₆ cycloalkyl). In some embodiments, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group and/or a R¹ and R⁹ group and/or a R² and R⁹ group and/or a R³ and R⁹ group and/or a R⁹ and R⁴ group and/or a R⁹ and R⁵ group, together with the atoms to which they are attached, combine to form an optionally substituted 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, only one pair combines to form an optionally substituted 3- to 8-membered saturated or partially unsaturated ring (i.e., a carbocyclyl or heterocyclyl having 1-2 heteroatoms independently selected from nitrogen, oxygen, and sulfur), wherein the pair is selected from a a R¹ and R² group; a R¹ and R³ group; a R¹ and R⁴ group; a R¹ and R⁵ group; a R² and R³ group; a R² and R⁴ group; a R² and R⁵ group; a R³ and R⁴ group; a R³ and R⁵ group; a R³ and R⁶ group; a R³ and R⁷ group; a R⁴ and R⁵ group; a R⁴ and R⁶ group; a R⁴ and R⁷ group; a R⁵ and R⁶ group; a R⁵ and R⁷ group; a R¹ and R⁹ group; a R² and R⁹ group; a R³ and R⁹ group; a R⁹ and R⁴ group; and a R⁹ and R⁵ group. In some embodiments, only one pair combines to form a 3- to 8-membered saturated or partially unsaturated ring (i.e., a carbocyclyl or heterocyclyl having 1-2 heteroatoms independently selected from nitrogen, oxygen, and sulfur), wherein the pair is selected from a R¹ and R² group; a R¹ and R³ group; a R¹ and R⁴ group; a R¹ and R⁵ group; a R² and R³ group; a R² and R⁴ group; a R² and R⁵ group; a R³ and R⁴ group; a R³ and R⁵ group; R³ and R⁶ group; a R³ and R⁷ group; and a R⁴ and R⁵ group.

In some embodiments, a R¹ and R² group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R² group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R² group, together with the atoms to which they are attached, combine to form a 3- to 6-membered saturated or partially unsaturated carbocyclyl (e.g., a C₃₋₆ cycloalkyl). In some embodiments, a R¹ and R² group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R¹ and R³ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R³ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R³ group, together with the atoms to which they are attached, combine to form a 3- to 6-membered saturated or partially unsaturated carbocyclyl (e.g., a C₃₋₆ cycloalkyl). In some embodiments, a R¹ and R³ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R¹ and R⁴ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁴ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁴ group, together with the atoms to which they are attached, combine to form a 3- to 6-membered saturated or partially unsaturated carbocyclyl (e.g., a C₃₋₆ cycloalkyl). In some embodiments, a R¹ and R⁴ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R¹ and R⁵ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁵ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁵ group, together with the atoms to which they are attached, combine to form a 3- to 6-membered saturated or partially unsaturated carbocyclyl (e.g., a C₃₋₆ cycloalkyl). In some embodiments, a R¹ and R⁵ group, together with the atoms to which they are attached, combine to form a 5-membered saturated or partially unsaturated carbocyclyl (e.g., a C₅ cycloalkyl). In some embodiments, a R¹ and R⁵ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R¹ and R⁵ group, together with the atoms to which they are attached, combine to form a 5-membered saturated or partially unsaturated heterocyclyl having 1-2 heteroatoms independently selected from nitrogen, oxygen, and sulfur (e.g., a 5-membered saturated heterocyclyl).

In some embodiments, a R² and R³ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R² and R³ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R² and R³ group, together with the atoms to which they are attached, combine to form a 3- to 6-membered saturated or partially unsaturated carbocyclyl (e.g., a C₃₋₆ cycloalkyl). In some embodiments, a R² and R³ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R² and R⁴ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁴ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁴ group, together with the atoms to which they are attached, combine to form a 3- to 6-membered saturated or partially unsaturated carbocyclyl (e.g., a C₃₋₆ cycloalkyl). In some embodiments, a R² and R⁴ group, together with the atoms to which they are attached, combine to form a 5-membered saturated or partially unsaturated carbocyclyl (e.g., a C₅ cycloalkyl). In some embodiments, a R² and R⁴ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R² and R⁴ group, together with the atoms to which they are attached, combine to form a 5-membered saturated or partially unsaturated heterocyclyl having 1-2 heteroatoms independently selected from nitrogen, oxygen, and sulfur (e.g., a 5-membered saturated heterocyclyl).

In some embodiments, a R² and R⁵ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁵ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁵ group, together with the atoms to which they are attached, combine to form a 3- to 6-membered saturated or partially unsaturated carbocyclyl (e.g., a C₃₋₆ cycloalkyl). In some embodiments, a R² and R⁵ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R³ and R⁴ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁴ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁴ group, together with the atoms to which they are attached, combine to form a 3- to 6-membered saturated or partially unsaturated carbocyclyl (e.g., a C₃₋₆ cycloalkyl). In some embodiments, a R³ and R⁴ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R³ and R⁵ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁵ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁵ group, together with the atoms to which they are attached, combine to form a 3- to 6-membered saturated or partially unsaturated carbocyclyl (e.g., a C₃₋₆ cycloalkyl). In some embodiments, a R³ and R⁵ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R³ and R⁶ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁶ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁶ group, together with the atoms to which they are attached, combine to form a 3- to 6-membered saturated or partially unsaturated carbocyclyl (e.g., a C₃₋₆ cycloalkyl). In some embodiments, a R³ and R⁶ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R³ and R⁷ group, together with the atoms to which they are attached, combine to form an optionally substituted 3- to 8-membered saturated or partially unsaturated ring (i.e., a heterocyclyl having 1-2 heteroatoms independently selected from nitrogen, oxygen, and sulfur). In some embodiments, a R³ and R⁷ group, together with the atoms to which they are attached, combine to form a 3- to 8-membered saturated or partially unsaturated ring (i.e., a heterocyclyl having 1-2 heteroatoms independently selected from nitrogen, oxygen, and sulfur). In some embodiments, a R³ and R⁷ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R³ and R⁷ group, together with the atoms to which they are attached, combine to form a 4-membered saturated or partially unsaturated heterocyclyl having 1-2 heteroatoms independently selected from nitrogen, oxygen, and sulfur (e.g., a 4-membered saturated heterocyclyl). In some embodiments, a R³ and R⁷ group, together with the atoms to which they are attached, combine to form a 5-membered saturated or partially unsaturated heterocyclyl having 1-2 heteroatoms independently selected from nitrogen, oxygen, and sulfur (e.g., a 5-membered saturated heterocyclyl), wherein the heterocyclyl is optionally substituted with one or more C₁₋₆ alkyl (e.g., methyl). In some embodiments, a R³ and R⁷ group, together with the atoms to which they are attached, combine to form a 5-membered saturated or partially unsaturated heterocyclyl having 1-2 heteroatoms independently selected from nitrogen, oxygen, and sulfur (e.g., a 5-membered saturated heterocyclyl).

In some embodiments, a R⁴ and R⁵ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁵ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁵ group, together with the atoms to which they are attached, combine to form a 3- to 6-membered saturated or partially unsaturated carbocyclyl (e.g., a C₃₋₆ cycloalkyl). In some embodiments, a R⁴ and R⁵ group, together with the atoms to which they are attached, combine to form a 6-membered saturated or partially unsaturated carbocyclyl (e.g., a 6-membered saturated carbocyclyl). In some embodiments, a R⁴ and R⁵ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R⁴ and R⁵ group, together with the atoms to which they are attached, combine to form a 5- to 7-membered saturated or partially unsaturated heterocyclyl having 1-2 heteroatoms independently selected from nitrogen, oxygen, and sulfur (e.g., a 5- to 7-membered saturated heterocyclyl). In some embodiments, a R⁴ and R⁵ group, together with the atoms to which they are attached, combine to form a 7-membered saturated or partially unsaturated heterocyclyl having 1-2 heteroatoms independently selected from nitrogen, oxygen, and sulfur (e.g., a 7-membered saturated heterocyclyl).

In some embodiments, a R⁴ and R⁶ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁶ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁶ group, together with the atoms to which they are attached, combine to form a 3- to 6-membered saturated or partially unsaturated carbocyclyl (e.g., a C₃₋₆ cycloalkyl). In some embodiments, a R⁴ and R⁶ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R⁴ and R⁷ group, together with the atoms to which they are attached, combine to form an optionally substituted 3- to 8-membered saturated or partially unsaturated ring (i.e., a heterocyclyl having 1-2 heteroatoms independently selected from nitrogen, oxygen, and sulfur). In some embodiments, a R⁴ and R⁷ group, together with the atoms to which they are attached, combine to form a 3- to 8-membered saturated or partially unsaturated ring (i.e., a heterocyclyl having 1-2 heteroatoms independently selected from nitrogen, oxygen, and sulfur). In some embodiments, a R⁴ and R⁷ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R⁴ and R⁷ group, together with the atoms to which they are attached, combine to form a 6-membered saturated or partially unsaturated heterocyclyl having 1-2 heteroatoms independently selected from nitrogen, oxygen, and sulfur (e.g., a 6-membered saturated heterocyclyl, such as morpholine).

In some embodiments, a R⁵ and R⁶ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R⁵ and R⁶ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R⁵ and R⁶ group, together with the atoms to which they are attached, combine to form a 3- to 6-membered saturated or partially unsaturated carbocyclyl (e.g., a C₃₋₆ cycloalkyl). In some embodiments, a R⁵ and R⁶ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R⁵ and R⁷ group, together with the atoms to which they are attached, combine to form an optionally substituted 3- to 8-membered saturated or partially unsaturated ring (i.e., a heterocyclyl having 1-2 heteroatoms independently selected from nitrogen, oxygen, and sulfur). In some embodiments, a R⁵ and R⁷ group, together with the atoms to which they are attached, combine to form a 3- to 8-membered saturated or partially unsaturated ring (i.e., a heterocyclyl having 1-2 heteroatoms independently selected from nitrogen, oxygen, and sulfur). In some embodiments, a R⁵ and R⁷ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R⁵ and R⁷ group, together with the atoms to which they are attached, combine to form a 6-membered saturated or partially unsaturated heterocyclyl having 1-2 heteroatoms independently selected from nitrogen, oxygen, and sulfur (e.g., a 6-membered saturated heterocyclyl, such as morpholine).

In some embodiments, a R¹ and R⁹ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁹ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R¹ and R⁹ group, together with the atoms to which they are attached, combine to form a 3- to 6-membered saturated or partially unsaturated carbocyclyl (e.g., a C₃₋₆ cycloalkyl). In some embodiments, a R¹ and R⁹ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R² and R⁹ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁹ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R² and R⁹ group, together with the atoms to which they are attached, combine to form a 3- to 6-membered saturated or partially unsaturated carbocyclyl (e.g., a C₃₋₆ cycloalkyl). In some embodiments, a R² and R⁹ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R³ and R⁹ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁹ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R³ and R⁹ group, together with the atoms to which they are attached, combine to form a 3- to 6-membered saturated or partially unsaturated carbocyclyl (e.g., a C₃₋₆ cycloalkyl). In some embodiments, a R³ and R⁹ group, together with the atoms to which they are attached, combine to form a 6-membered saturated or partially unsaturated carbocyclyl (e.g., a 6-membered saturated carbocyclyl). In some embodiments, a R³ and R⁹ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl). In some embodiments, a R³ and R⁹ group, together with the atoms to which they are attached, combine to form a 6-membered saturated or partially unsaturated heterocyclyl having 1-2 heteroatoms independently selected from nitrogen, oxygen, and sulfur (e.g., a 6-membered saturated heterocyclyl).

In some embodiments, a R⁴ and R⁹ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁹ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R⁴ and R⁹ group, together with the atoms to which they are attached, combine to form a 3- to 6-membered saturated or partially unsaturated carbocyclyl (e.g., a C₃₋₆ cycloalkyl). In some embodiments, a R⁴ and R⁹ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments, a R⁵ and R⁹ group, together with the atoms to which they are attached, combine to form an optionally substituted 3- to 8-membered saturated or partially unsaturated ring (i.e., a carbocyclyl or heterocyclyl having 1-2 heteroatoms independently selected from nitrogen, oxygen, and sulfur). In some embodiments, a R⁵ and R⁹ group, together with the atoms to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, a R⁵ and R⁹ group, together with the atoms to which they are attached, combine to form a 3- to 6-membered saturated or partially unsaturated carbocyclyl (e.g., a C₃₋₆ cycloalkyl). In some embodiments, a R⁵ and R⁹ group, together with the atoms to which they are attached, combine to form a 3- to 6-membered saturated or partially unsaturated heterocyclyl having 1-2 heteroatoms independently selected from nitrogen, oxygen, and sulfur (e.g., a 3- to 6-membered saturated heterocyclyl).

In some embodiments of any of Formulae I, II, III, IV, and V, a moiety

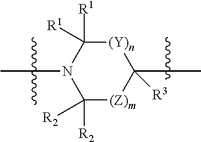

is selected from:

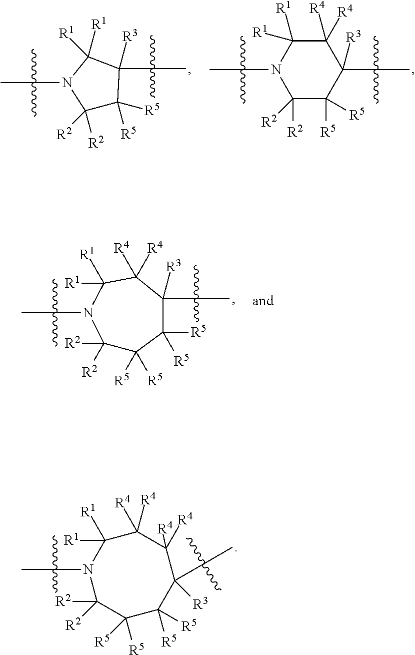

In some embodiments, a moiety

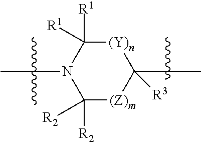

is selected from:

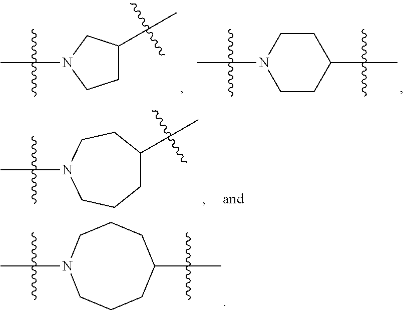

In some embodiments, a moiety

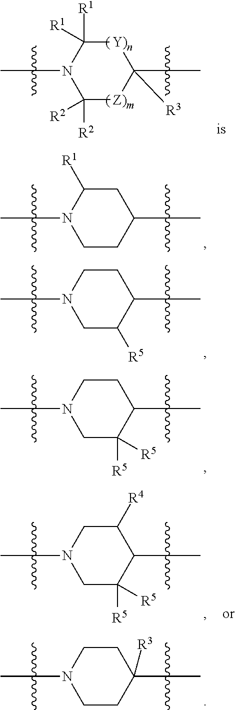

In some embodiments, a moiety

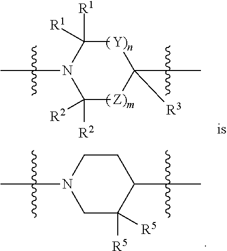

In some embodiments, a moiety

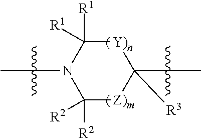

is selected from

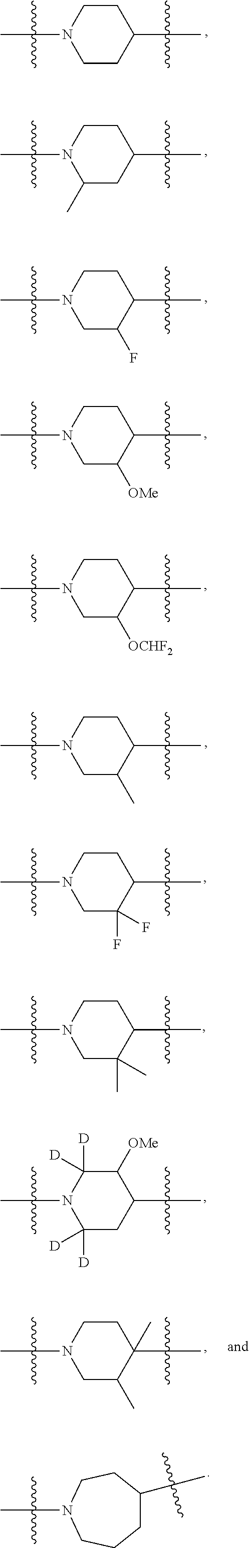

In some embodiments, a moiety

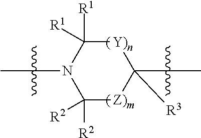

is selected from

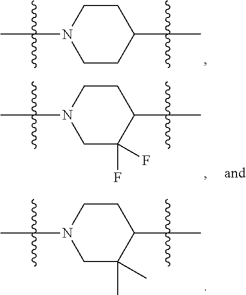

In some embodiments, a moiety

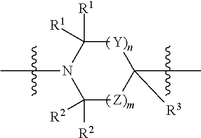

is selected from

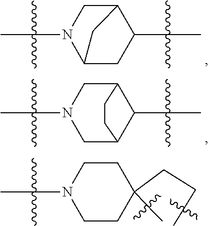

(i.e., when R³ combines with R⁷ to form a 4-membered ring),

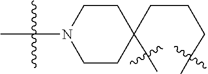 [SMILES: *CCCC1(*)CCN(*)CC1]

(i.e., when R³ combines with R⁷ to form a 5-membered ring),

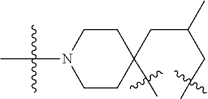 [SMILES: *CC(C)CC1(*)CCN(*)CC1]

(i.e., when R³ combines with R⁷ to form a 5-membered ring substituted with a methyl group)

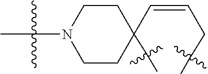 [SMILES: *C/C=C\C1(*)CCN(*)CC1]

(i.e., when R³ combines with R⁷ to form a 5-membered ring), and

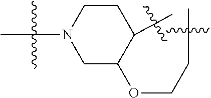 [SMILES: *CCOC1CN(*)CCC1*]

(i.e., when R⁴ combines with R⁷ to form a 6-membered ring). In some embodiments, a moiety

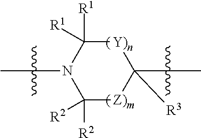

is selected from

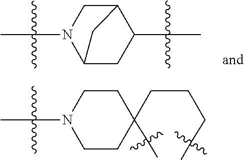

(i.e. when R³ combines with R⁷ to form a 5-membered ring).

In some embodiments of Formula VI, a moiety

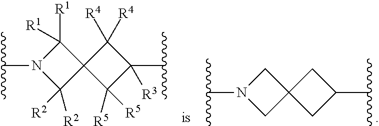

In some embodiments of Formula VII, a moiety

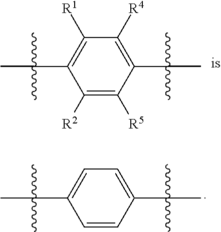

In some embodiments of Formula VIII, a moiety

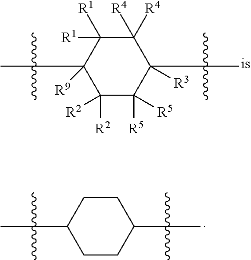

In some embodiments of Formula IX, a moiety

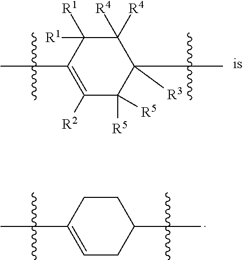

In some embodiments of Formula XI, a moiety

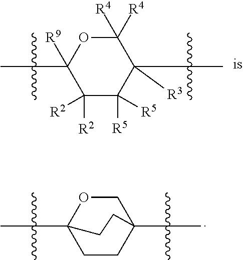

In some embodiments of any of Formulae A, B, C, D, E, I, IA, IB, IC, ID, II, III, IV, V, VI, VII, VIII, IX, X, and XI, each R⁶ is independently hydrogen or optionally substituted C₁₋₆ aliphatic. In some embodiments, each R⁶ is hydrogen, or two R⁶ groups, together with the atom to which they are attached, combine to form a 3- to 8-membered saturated or partially unsaturated ring. In some embodiments, each R⁶ is hydrogen. In some embodiments, R⁶ is hydrogen. In some embodiments, R⁶ is optionally substituted C₁₋₆ aliphatic (e.g., optionally substituted C₁₋₆ alkyl). In some embodiments, two R⁶ groups, together with the atom to which they are attached, combine to form a 3- to 8-membered saturated or partially unsaturated ring (i.e., a carbocyclyl or heterocyclyl having 1-2 heteroatoms independently selected from nitrogen, oxygen, and sulfur). In some embodiments, two R⁶ groups, together with the atom to which they are attached, combine to form a 3- to 6-membered saturated or partially unsaturated carbocyclyl. In some embodiments, two R⁶ groups, together with the atom to which they are attached, combine to form a C₃₋₆ cycloalkyl (e.g., cyclopropyl). In some embodiments, two R⁶ groups, together with the atom to which they are attached, combine to form a 3- to 6-membered saturated or partially unsaturated heterocyclyl having 1-2 heteroatoms independently selected from nitrogen, oxygen, and sulfur. In some embodiments, two R⁶ groups, together with the atom to which they are attached, combine to form a 3- to 6-membered saturated heterocyclyl having 1-2 heteroatoms independently selected from nitrogen, oxygen, and sulfur.

In some embodiments of any of Formulae A, B, C, D, E, I, IA, IB, IC, ID, II, III, IV, V, VI, VII, VIII, IX, X, and XI, R⁷ is optionally substituted C₁₋₆ aliphatic. In some embodiments, R⁷ is optionally substituted C₁₋₆ alkyl. In some embodiments, R⁷ is C₁₋₆ alkyl. In some embodiments, R⁷ is optionally substituted C₁₋₄ alkyl (e.g., methyl). In some embodiments, R⁷ is hydrogen.

In some embodiments of any of Formulae A, B, C, I, IA, IB, IC, ID, II, III, VI, VII, VIII, IX, X, and XI, Ring A is an optionally substituted group selected from 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur. In some embodiments, Ring A is an optionally substituted group selected from phenyl, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur. In some embodiments, Ring A is an optionally substituted group selected from phenyl and 5- to 6-membered monocyclic heteroaryl having 1-4 heteroatoms independently selected from nitrogen, oxygen, and sulfur. In some embodiments, Ring A is phenyl or 5- to 6-membered monocyclic heteroaryl having 1-4 heteroatoms independently selected from nitrogen, oxygen, and sulfur.

In some embodiments, Ring A is phenyl.

In some embodiments, Ring A is a 5- to 6-membered monocyclic heteroaryl having 1-4 heteroatoms independently selected from nitrogen, oxygen, and sulfur. In some embodiments, Ring A is a 5- to 6-membered monocyclic heteroaryl having 1-2 heteroatoms independently selected from nitrogen, oxygen, and sulfur. In some embodiments, Ring A is a 5-membered monocyclic heteroaryl having 1-4 heteroatoms independently selected from nitrogen, oxygen, and sulfur. In some embodiments, Ring A is pyrazolyl. In some embodiments, Ring A is a 6-membered monocyclic heteroaryl having 1-4 heteroatoms independently selected from nitrogen, oxygen, and sulfur. In some embodiments, Ring A is pyridyl or pyridonyl.

In some embodiments, Ring A is a 8- to 10-membered bicyclic heteroaryl having 1-4 heteroatoms independently selected from nitrogen, oxygen, and sulfur. In some embodiments, Ring A is a 8-membered bicyclic heteroaryl having 1-4 heteroatoms independently selected from nitrogen, oxygen, and sulfur. In some embodiments, Ring A is a 9-membered bicyclic heteroaryl having 1-4 heteroatoms independently selected from nitrogen, oxygen, and sulfur. In some embodiments, Ring A is a 10-membered bicyclic heteroaryl having 1-4 heteroatoms independently selected from nitrogen, oxygen, and sulfur.

In some embodiments, Ring A is a 3- to 7-membered saturated or partially unsaturated monocyclic carbocyclyl. In some embodiments, Ring A is a 3-membered saturated or partially unsaturated monocyclic carbocyclyl. In some embodiments, Ring A is a 4-membered saturated or partially unsaturated monocyclic carbocyclyl. In some embodiments, Ring A is a 5-membered saturated or partially unsaturated monocyclic carbocyclyl. In some embodiments, Ring A is a 6-membered saturated or partially unsaturated monocyclic carbocyclyl. In some embodiments, Ring A is a 7-membered saturated or partially unsaturated monocyclic carbocyclyl.

In some embodiments, Ring A is a 3- to 7-membered saturated or partially unsaturated monocyclic heterocyclyl having 1-3 heteroatoms independently selected from nitrogen, oxygen, and sulfur. In some embodiments, Ring A is a 3-membered saturated or partially unsaturated monocyclic heterocyclyl having 1-2 heteroatoms independently selected from nitrogen, oxygen, and sulfur. In some embodiments, Ring A is a 4-membered saturated or partially unsaturated monocyclic heterocyclyl having 1-3 heteroatoms independently selected from nitrogen, oxygen, and sulfur. In some embodiments, Ring A is a 5-membered saturated or partially unsaturated monocyclic heterocyclyl having 1-3 heteroatoms independently selected from nitrogen, oxygen, and sulfur. In some embodiments, Ring A is a 6-membered saturated or partially unsaturated monocyclic heterocyclyl having 1-3 heteroatoms independently selected from nitrogen, oxygen, and sulfur. In some embodiments, Ring A is a 7-membered saturated or partially unsaturated monocyclic heterocyclyl having 1-3 heteroatoms independently selected from nitrogen, oxygen, and sulfur.

In some embodiments, Ring A is a 7- to 10-membered saturated or partially unsaturated bicyclic heterocyclyl having 1-4 heteroatoms independently selected from nitrogen, oxygen, and sulfur. In some embodiments, Ring A is a 7-membered saturated or partially unsaturated bicyclic heterocyclyl having 1-4 heteroatoms independently selected from nitrogen, oxygen, and sulfur. In some embodiments, Ring A is a 8-membered saturated or partially unsaturated bicyclic heterocyclyl having 1-4 heteroatoms independently selected from nitrogen, oxygen, and sulfur. In some embodiments, Ring A is a 9-membered saturated or partially unsaturated bicyclic heterocyclyl having 1-4 heteroatoms independently selected from nitrogen, oxygen, and sulfur. In some embodiments, Ring A is a 10-membered saturated or partially unsaturated bicyclic heterocyclyl having 1-4 heteroatoms independently selected from nitrogen, oxygen, and sulfur.

In some embodiments, Ring A is (i) optionally substituted on a substitutable carbon atom with one or more groups independently selected from oxo, halogen, —(CH₂)₀₋₄R^{o}, —CN, —OR^{o}, —O(CH₂)₁₋₄R^{o}, —SR^{o}, —N(R^{o})₂, —NO₂, —C(O)R^{o}, —C(O)OR^{o}, —C(O)NR^{o}₂, —OC(O)R^{o}, —OC(O)NR^{o}₂, —OC(O)OR^{o}, —OS(O)₂R^{o}, —OS(O)₂NR^{o}₂, —N(R^{o})C(O)R^{o}, —N(R^{o})S(O)₂R^{o}, —S(O)₂R^{o}, —SO₂NR^{o}₂, and —S(O)₂OR^{o}, and (ii) optionally substituted on a substitutable nitrogen atom with one or more groups selected from —R, —NR^{†}₂, —C(O)R^{†}, —C(O)OR^{†}, —S(O)₂R^{†}, and —S(O)₂NR^{†}₂. In some embodiments, Ring A is (i) optionally substituted on a substitutable carbon atom with one or more groups independently selected from oxo, —(CH₂)₀₋₄R^{o}, and —N(R^{o})₂; and (ii) optionally substituted on a substitutable nitrogen atom with one or more groups selected from —R^{†}.

In some embodiments of any of Formulae A, B, C, D, E, I, IA, IB, IC, ID, II, III, IV, V, VI, VII, VIII, IX, X, and XI, L is a covalent bond. In some embodiments, L is a bivalent C₁₋₃ straight or branched hydrocarbon chain. In some embodiments, L is a bivalent C₁₋₂ straight or branched hydrocarbon chain. In some embodiments, L is methylene (i.e., —CH₂—). In some embodiments, L is —CH₂CH₂—. In some embodiments, L is —CH₂CH₂CH₂—. In some embodiments, L is —C(CH₃)₂—. In some embodiments, L is a covalent bond or —CH₂—.

In some embodiments of any of Formulae A, B, C, D, E, I, IA, IB, IC, ID, II, III, IV, V, VI, VII, VIII, IX, X, and XI, R⁸ is halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur. In some embodiments, R⁸ is optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur. In some embodiments, R⁸ is optionally substituted C₁₋₆ aliphatic or optionally substituted 3- to 6-membered saturated or partially unsaturated monocyclic heterocyclyl having 1-3 heteroatoms independently selected from nitrogen, oxygen, and sulfur. In some embodiments, R⁸ is optionally substituted C₁₋₆ alkyl, optionally substituted C₃₋₆ cycloalkyl, or optionally substituted 3- to 6-membered saturated monocyclic heterocyclyl having 1-3 heteroatoms independently selected from nitrogen, oxygen, and sulfur. In some embodiments, R⁸ is optionally substituted C₁₋₆ alkyl or optionally substituted 3- to 6-membered saturated monocyclic heterocyclyl having 1-3 heteroatoms independently selected from nitrogen, oxygen, and sulfur.

In some embodiments, R⁸ is hydrogen. In some embodiments, R⁸ is not hydrogen.

In some embodiments, R⁸ is halogen. In some embodiments, R⁸ is fluoro. In some embodiments, R⁸ is chloro.

In some embodiments, R⁸ is optionally substituted C₁₋₆ aliphatic. In some embodiments, R⁸ is optionally substituted straight-chain or branched C₁₋₆ aliphatic (i.e., optionally substituted acyclic C₁₋₆ aliphatic). In some embodiments, R⁸ is C₁₋₆ aliphatic optionally substituted with one or more halogen (e.g., fluoro). In some embodiments, R⁸ is optionally substituted C₁₋₆ alkyl. In some embodiments, R⁸ is C₁₋₆ alkyl optionally substituted with one or more halogen (e.g., fluoro). In some embodiments, R⁸ is unsubstituted C₁₋₆ alkyl (e.g., methyl). In some embodiments, R⁸ is C₁₋₆ haloalkyl (e.g., —CF₃).

In some embodiments, R⁸ is optionally substituted C₃₋₆ cycloaliphatic. In some embodiments, R⁸ is optionally substituted C₃₋₆ cycloalkyl. In some embodiments, R⁸ is optionally substituted C₃ cycloaliphatic. In some embodiments, R⁸ is optionally substituted C₄ cycloaliphatic (e.g., cyclobutane optionally substituted with one or more —OH). In some embodiments, R⁸ is optionally substituted C₅ cycloaliphatic. In some embodiments, R⁸ is optionally substituted C₆ cycloaliphatic.

In some embodiments, R⁸ is optionally substituted 3- to 6-membered saturated or partially unsaturated monocyclic heterocyclyl having 1-3 heteroatoms independently selected from nitrogen, oxygen, and sulfur. In some embodiments, R⁸ is optionally substituted 4- to 6-membered saturated monocyclic heterocyclyl having 1-2 heteroatoms independently selected from nitrogen, oxygen, and sulfur. In some embodiments, R⁸ is 4- to 6-membered saturated monocyclic heterocyclyl having 1-2 heteroatoms independently selected from nitrogen, oxygen, and sulfur, and optionally substituted with one or more C₁₋₆ alkyl or —OH. In some embodiments, R⁸ is optionally substituted 3-membered saturated monocyclic heterocyclyl having 1 heteroatom independently selected from nitrogen, oxygen, and sulfur. In some embodiments, R⁸ is optionally substituted 4-membered saturated monocyclic heterocyclyl having 1 heteroatom independently selected from nitrogen, oxygen, and sulfur. In some embodiments, R⁸ is optionally substituted 5-membered saturated monocyclic heterocyclyl having 1-2 heteroatoms independently selected from nitrogen, oxygen, and sulfur. In some embodiments, R⁸ is optionally substituted tetrahydrofuranyl. In some embodiments, R⁸ is optionally substituted 6-membered saturated monocyclic heterocyclyl having 1-3 heteroatoms independently selected from nitrogen, oxygen, and sulfur. In some embodiments, R⁸ is optionally substituted tetrahydropyranyl, piperidinyl, or piperazinyl. In some embodiments, R⁸ is optionally substituted piperidinyl or piperazinyl.

In some embodiments, R⁸ is optionally substituted phenyl. In some embodiments, R′ is not optionally substituted phenyl.

In some embodiments, R⁸ is optionally substituted 5- to 6-membered monocyclic heteroaryl having 1-4 heteroatoms independently selected from nitrogen, oxygen, and sulfur. In some embodiments, R⁸ is optionally substituted 5- to 6-membered monocyclic heteroaryl having 1-2 heteroatoms independently selected from nitrogen, oxygen, and sulfur. In some embodiments, R⁸ is optionally substituted 5-membered monocyclic heteroaryl having 1-4 heteroatoms independently selected from nitrogen, oxygen, and sulfur. In some embodiments, R⁸ is optionally substituted 6-membered monocyclic heteroaryl having 1-4 heteroatoms independently selected from nitrogen, oxygen, and sulfur.

In some embodiments, R⁸ is optionally substituted 7- to 10-membered saturated or partially unsaturated bicyclic heteroaryl having 1-4 heteroatoms independently selected from nitrogen, oxygen, and sulfur. In some embodiments, R⁸ is optionally substituted 7- to 10-membered saturated, spirocyclic, bicyclic heterocyclyl having 1-4 heteroatoms independently selected from nitrogen, oxygen, and sulfur.

In some embodiments of any of Formulae A, B, C, D, E, I, IA, IB, IC, ID, II, III, IV, V, VI, VII, VIII, IX, X, and XI,

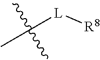

is R⁸ is —R⁸ (i.e., L is a covalent bond). In some embodiments,

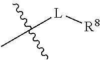

is —(C₁₋₃ alkylene)-R⁸ (i.e., L is a C₁₋₃ straight or branched hydrocarbon chain). In some embodiments,

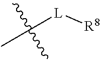

is —(C₁₋₂ alkylene)-R⁸ (i.e., L is a C₁₋₂ straight or branched hydrocarbon chain). In some embodiments,

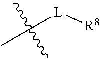

is —CH₂—R⁸ (i.e., L is a C₁ hydrocarbon chain). In some embodiments,

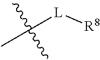

—CH₂CH₂—R⁸ (i.e., L is a C₂ straight hydrocarbon chain). In some embodiments,

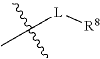

is —CH₂CH₂CH₂—R⁸ (i.e., L is a C₃ straight hydrocarbon chain). In some embodiments

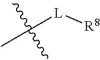

is —C(CH₃)₂—R⁸ (i.e., L is a C₃ branched hydrocarbon chain).

In some embodiments of any of Formulae A, B, C, D, E, I, IA, IB, IC, ID, II, III, IV, V, VI, VII, VIII, IX, X, and XI, each R^{a} is independently halogen, —CN, —OR, —O(CH₂)₁₋₄R, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.

In some embodiments, R^{a} is halogen. In some embodiments, R^{a} is fluoro. In some embodiments, R^{a} is chloro.

In some embodiments, R^{a} is —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, or —SO₃R′. In some embodiments, R^{a} is —CN, —OR, or —O(CH₂)₁₋₄R.

In some embodiments, R^{a} is optionally substituted C₁₋₆ aliphatic. In some embodiments, R^{a} is optionally substituted straight-chain or branched C₁₋₆ aliphatic (i.e., optionally substituted acyclic C₁₋₆ aliphatic). In some embodiments, R^{a} is optionally substituted C₁₋₆ alkyl. In some embodiments, R^{a} is C₁₋₆ alkyl optionally substituted with one or more halogen (e.g., fluoro). In some embodiments, R^{a} is optionally substituted C₁₋₄ alkyl. In some embodiments, R^{a} is C₁₋₄ alkyl optionally substituted with one or more halogen (e.g., fluoro). In some embodiments, R^{a} is C₁₋₄ haloalkyl (e.g., —CF₃). In some embodiments, R^{a} is unsubstituted C₁₋₄ alkyl (e.g., methyl or tert-butyl). In some embodiments, R^{a} is —CH₃, —CF₃, or —C(CH₃)₃. In some embodiments, R^{a} is —CH₃ or —CF₃.

In some embodiments, R^{a} is optionally substituted C₃₋₆ cycloaliphatic. In some embodiments, R^{a} is optionally substituted C₃₋₆ cycloalkyl. In some embodiments, R^{a} is optionally substituted cyclopropyl.

In some embodiments, R^{a} is optionally substituted 3- to 6-membered saturated or partially unsaturated monocyclic heterocyclyl having 1-3 heteroatoms independently selected from nitrogen, oxygen, and sulfur. In some embodiments, R^{a} is optionally substituted 3- to 6-membered saturated monocyclic heterocyclyl having 1-2 heteroatoms independently selected from nitrogen, oxygen, and sulfur.

In some embodiments, R^{a} is optionally substituted phenyl.

In some embodiments, R^{a} is optionally substituted 5- to 6-membered monocyclic heteroaryl having 1-4 heteroatoms independently selected from nitrogen, oxygen, and sulfur.

In some embodiments of any of Formulae A, B, C, D, E, I, IA, IB, IC, ID, II, III, IV, V, VI, VII, VIII, IX, X, and XI, p is 0, 1, or 2. In some embodiments, p is 0 or 1. In some embodiments, p is 0. In some embodiments, p is 1. In some embodiments, p is 2. In some embodiments, p is 3. In some embodiments, p is 4. In some embodiments, p is 5.

In some embodiments of any of Formulae A, B, C, I, IA, IB, IC, ID, II, III, VI, VII, VIII, IX, X, and XI, a moiety

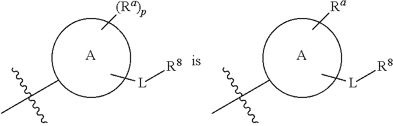

In some embodiments, a moiety

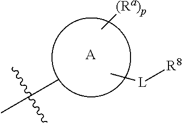

selected from:

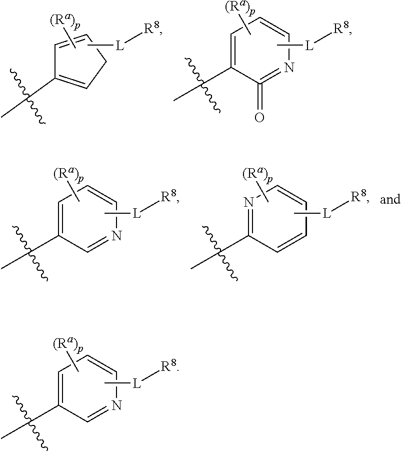

In some embodiments a moiety

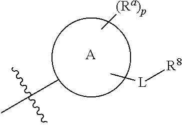

is selected from:

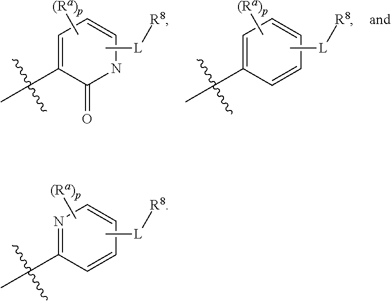

In some embodiments a moiety

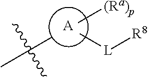

is selected from:

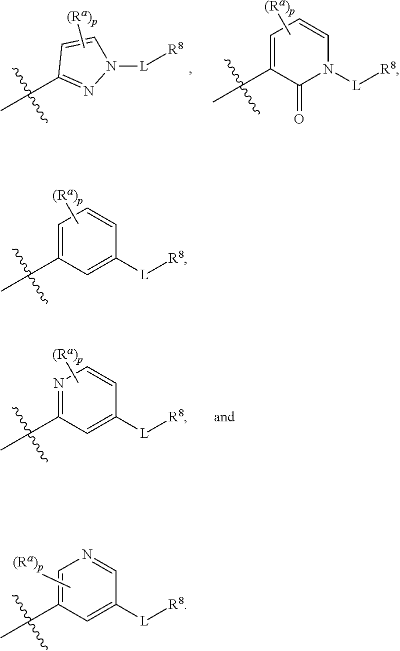

In some embodiments, a moiety

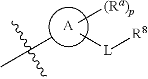

is selected from:

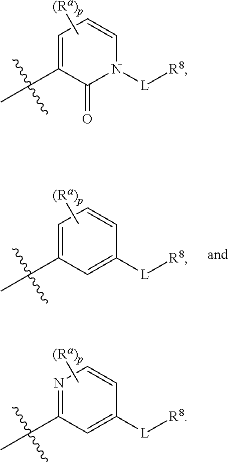

In some embodiments, a moiety

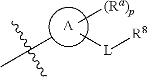

is selected from:

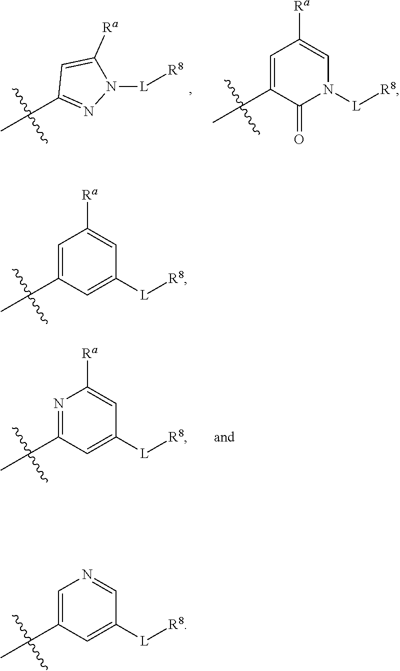

In some embodiments a moiety

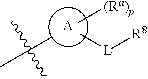

is selected from:

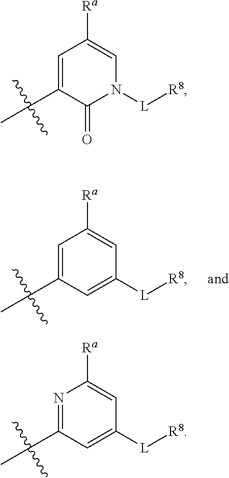

In some embodiments, a moiety

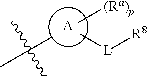

is selected from:

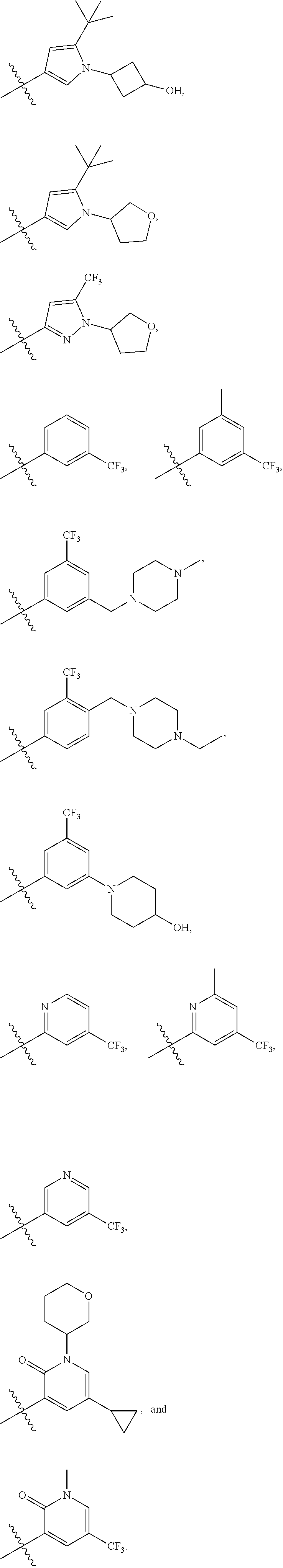

In some embodiments of any of Formulae A, D, E, I, IA, IB, IC, ID, IV, V, VI, VII, VIII, IX, X, and XI, Ring B is an optionally substituted group selected from 5- to 6-membered monocyclic heteroaryl having 1-4 heteroatoms independently selected from nitrogen, oxygen, and sulfur and 8- to 10-membered bicyclic heteroaryl having 1-4 heteroatoms independently selected from nitrogen, oxygen, and sulfur. In some embodiments, Ring B is selected from 5- to 6-membered monocyclic heteroaryl having 1-4 heteroatoms independently selected from nitrogen, oxygen, and sulfur and 8- to 10-membered bicyclic heteroaryl having 1-4 heteroatoms independently selected from nitrogen, oxygen, and sulfur.

In some embodiments, Ring B is a 5- to 6-membered monocyclic heteroaryl having 1-4 heteroatoms independently selected from nitrogen, oxygen, and sulfur. In some embodiments, Ring B is a 5- to 6-membered monocyclic heteroaryl having 1-2 heteroatoms independently selected from nitrogen, oxygen, and sulfur. In some embodiments, Ring B is a 5-membered monocyclic heteroaryl having 1-2 heteroatoms independently selected from nitrogen, oxygen, and sulfur. In some embodiments, Ring B is a 6-membered monocyclic heteroaryl having 1-2 heteroatoms independently selected from nitrogen, oxygen, and sulfur. In some embodiments, Ring B is pyridinyl or pyrazinyl.

In some embodiments, Ring B is a 8- to 10-membered bicyclic heteroaryl having 1-4 heteroatoms independently selected from nitrogen, oxygen, and sulfur. In some embodiments, Ring B is a 8-membered bicyclic heteroaryl having 1-4 heteroatoms independently selected from nitrogen, oxygen, and sulfur. In some embodiments, Ring B is a 9-membered bicyclic heteroaryl having 1-4 heteroatoms independently selected from nitrogen, oxygen, and sulfur. In some embodiments, Ring B is 1H-pyrrolo[2,3-b]pyridinyl, 1H-pyrazolo[3,4-b]pyridinyl, 5H-pyrrolo[2,3-b]pyrazinyl, 3H-imidazo[4,5-b]pyridyl, pyrazolo[1,5-a]pyrimidyl, isothiazolo[5,4-b]pyridyl, 1H-pyrazolo[3,4-b]pyrazinyl, 1H-pyrazolo[4,3-b]pyridyl, 1,3-dihydro-2H-imidazo[4,5-b]pyridine-2-onyl, 1,2-dihydro-3H-pyrazolo[3,4-b]pyridine-3-onyl, imidazo[1,5-a]pyrimidinyl, pyrazole[1,5-a]pyrazinyl, or pyrrolo[1,2-b]pyridazinyl. In some embodiments, Ring B is a 10-membered bicyclic heteroaryl having 1-4 heteroatoms independently selected from nitrogen, oxygen, and sulfur.

In some embodiments, Ring B is a 10- to 16-membered polycyclic heteroaryl having 1-5 heteroatoms independently selected from nitrogen, oxygen, and sulfur.

In some embodiments, Ring B is (i) optionally substituted on a substitutable carbon atom with one or more groups independently selected from oxo, halogen, R^{o}, —CN, —OR^{o}, —SR^{o}, —N(R^{o})₂, —NO₂, —C(O)R^{o}, —C(O)OR^{o}, —C(O)NR^{o}₂, —OC(O)R^{o}, —OC(O)NR^{o}₂, —OC(O)OR^{o}, —OS(O)₂R^{o}, —OS(O)₂NR^{o}₂, —N(R^{o})C(O)R^{o}, —N(R^{o})C(O)NR^{o}₂, —N(R^{o})S(O)₂R^{o}, —S(O)₂R^{o}, —SO₂NR^{o}₂, and —S(O)₂OR^{o}, and (ii) optionally substituted on a substitutable nitrogen atom with one or more groups selected from —R, —NR^{†}₂, —C(O)R^{†}, —C(O)OR^{†}, —S(O)₂R^{†}, and —S(O)₂NR^{†}₂. In some embodiments, Ring B is (i) optionally substituted on a substitutable carbon atom with one or more groups independently selected from R^{o}, —N(R^{o})₂, —C(O)NR^{o}₂, —N(R^{o})C(O)R^{o}, and —N(R^{o})C(O)NR^{o}₂, and (ii) optionally substituted on a substitutable nitrogen with —R.

In some embodiments of any of Formulae A, B, C, D, E, I, IA, IB, IC, ID, II, III, IV, V, VI, VII, VIII, IX, X, and XI, each R^{b} is independently halogen, —CN, —OR, —N(R)₂, —C(O)N(R)₂, —N(R)C(O)R′, —N(R)C(O)N(R)₂, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur. In some embodiments, each R^{b} is independently halogen, —CN, —OR, —N(R)₂, —C(O)N(R)₂, —N(R)C(O)R′, —N(R)C(O)OR, —N(R)C(O)N(R)₂, optionally substituted C₁₋₆ aliphatic, or optionally substituted C₃₋₆ cycloaliphatic. In some embodiments, each R^{b} is independently halogen, —OR, —N(R)₂, —N(R)C(O)R′, —N(R)C(O)N(R)₂, optionally substituted C₁₋₆ aliphatic, or optionally substituted C₃₋₆ cycloaliphatic. In some embodiments, each R^{b} is independently halogen, —OR, —N(R)₂, —N(H)C(O)R′, —N(H)C(O)N(R)₂, optionally substituted C₁₋₆ alkyl, optionally substituted C₃₋₆ cycloalkyl, or optionally substituted 3- to 6-membered saturated monocyclic heterocyclyl having 1-3 heteroatoms independently selected from nitrogen, oxygen, and sulfur. In some embodiments, each R^{b} is independently halogen, —OR, —N(R)₂, —N(H)C(O)R′, —N(H)C(O)N(R)₂, optionally substituted C₁₋₆ alkyl, or optionally substituted C₃₋₆ cycloalkyl. In some embodiments, each R^{b} is independently halogen, —N(R)₂, optionally substituted C₁₋₆ aliphatic, or optionally substituted C₃₋₆ cycloaliphatic.

In some embodiments, R^{b} is halogen. In some embodiments, R^{b} is fluoro. In some embodiments, R^{b} is chloro.

In some embodiments, R^{b} is —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, or —SO₃R′. In some embodiments, R^{b} is —CN, —OR, —N(R)₂, —C(O)N(R)₂, —N(R)C(O)R′, or —N(R)C(O)OR, —N(R)C(O)N(R)₂.

In some embodiments, R^{b} is —CN.

In some embodiments, R^{b} is —OR. In some embodiments, R^{b} is —OR, wherein R of R^{b} is optionally substituted C₁₋₆ aliphatic, optionally substituted C₃₋₇ cycloaliphatic, or optionally substituted 3- to 7-membered saturated or partially unsaturated monocyclic heterocyclyl having 1-3 heteroatoms independently selected from nitrogen, oxygen, and sulfur. In some embodiments, R^{b} is —OR, wherein R of R^{b} is optionally substituted C₁₋₆ aliphatic (e.g., methyl, —CH₂CH₂OCH₃, —CH₂(1,4-dioxane), In some embodiments, R^{b} is —OR, wherein R of R^{b} is optionally substituted C₃₋₇ cycloaliphatic (e.g., cyclobutyl optionally substituted with one or more —OH). In some embodiments, R^{b} is —OR, wherein R of R^{b} is optionally substituted 3- to 7-membered saturated or partially unsaturated monocyclic heterocyclyl having 1-3 heteroatoms independently selected from nitrogen, oxygen, and sulfur (e.g., oxetanyl, tetrahydrofuranyl, or tetrahydropyranyl).

In some embodiments, R^{b} is —N(R)₂. In some embodiments, R^{b} is —N(R)₂, wherein each R of R^{b} is independently hydrogen or optionally substituted C₁₋₆ aliphatic (e.g., optionally substituted C₁₋₆ alkyl). In some embodiments, R^{b} is —NHCH₃ or —N(CH₃)₂.

In some embodiments, R^{b} is —C(O)N(R)₂. In some embodiments, R^{b} is —C(O)N(R)₂, wherein each R of R^{b} is independently hydrogen or optionally substituted C₁₋₆ aliphatic (e.g., optionally substituted C₁₋₆ alkyl). In some embodiments, R^{b} is —C(O)N(CH₃)₂.

In some embodiments, R^{b} is —N(R)C(O)R′. In some embodiments, R^{b} is —N(H)C(O)R′. In some embodiments, R^{b} is —N(H)C(O)(optionally substituted C₁₋₆ aliphatic). In some embodiments, R^{b} is —N(H)C(O)(C₁₋₆ alkyl). In some embodiments, R^{b} is —N(H)C(O)CH₃. In some embodiments, R^{b} is —N(H)C(O)(optionally substituted C₃₋₆ cycloaliphatic). In some embodiments, R^{b} is —N(H)C(O)(C₃₋₆ cycloalkyl). In some embodiments, R^{b} is —N(H)C(O)(cyclopropyl).

In some embodiments, R^{b} is —N(R)C(O)N(R)₂. In some embodiments, R^{b} is —N(H)C(O)N(R)₂. In some embodiments, R^{b} is —N(H)C(O)N(optionally substituted C₁₋₆ aliphatic)₂. In some embodiments, R^{b} is —N(H)C(O)N(C₁₋₆ alkyl)₂. In some embodiments, R^{b} is In some embodiments, R^{b} is —N(H)C(O)N(CH₃)₂.

In some embodiments, R^{b} is optionally substituted C₁₋₆ aliphatic. In some embodiments, R^{b} is optionally substituted straight-chain or branched C₁₋₆ aliphatic (i.e., optionally substituted acyclic C₁₋₆ aliphatic). In some embodiments, R^{b} is optionally substituted C₁₋₆ alkyl. In some embodiments, R^{b} is optionally substituted C₁₋₄ alkyl (e.g., methyl or —CH₂OCH₃). In some embodiments, R^{b} is unsubstituted C₁₋₄ alkyl (e.g., methyl).

In some embodiments, R^{b} is optionally substituted C₃₋₆ cycloaliphatic. In some embodiments, R^{b} is optionally substituted C₃₋₆ cycloalkyl. In some embodiments, R^{b} is cyclopropyl. In some embodiments, R^{b} is cyclobutyl. In some embodiments, R^{b} is cyclobutyl optionally substituted with —O(C₁₋₆ alkyl). In some embodiments, R^{b} is cyclopentyl. In some embodiments, R^{b} is cyclohexyl.

In some embodiments, R^{b} is optionally substituted 3- to 6-membered saturated or partially unsaturated monocyclic heterocyclyl having 1-3 heteroatoms independently selected from nitrogen, oxygen, and sulfur. In some embodiments, R^{b} is optionally substituted 3- to 6-membered saturated monocyclic heterocyclyl having 1-2 heteroatoms independently selected from nitrogen, oxygen, and sulfur. In some embodiments, R^{b} is optionally substituted 5-membered saturated monocyclic heterocyclyl having 1-2 heteroatoms independently selected from nitrogen, oxygen, and sulfur. In some embodiments, R^{b} is optionally substituted 6-membered saturated monocyclic heterocyclyl having 1-2 heteroatoms independently selected from nitrogen, oxygen, and sulfur. In some embodiments, R^{b} is optionally substituted piperidinyl, tetrahydropyranyl, or morpholinyl (e.g., piperidinyl, tetrahydropyranyl, or morpholinyl optionally substituted with C₁₋₆ alkyl or —C(O)(C₁₋₆ alkyl)).

In some embodiments, R^{b} is optionally substituted phenyl.

In some embodiments, R^{b} is optionally substituted 5- to 6-membered monocyclic heteroaryl having 1-4 heteroatoms independently selected from nitrogen, oxygen, and sulfur.

In some embodiments any of Formulae A, B, C, D, E, I, IA, IB, IC, ID, II, III, IV, V, VI, VII, VIII, IX, X, and XI, q is 0, 1, or 2. In some embodiments, q is 0 or 1. In some embodiments, q is 0. In some embodiments, q is 1. In some embodiments, q is 2. In some embodiments, q is 3. In some embodiments, q is 4. In some embodiments, q is 5.

In some embodiments of any of Formulae A, B, C, D, E, I, IA, IB, IC, ID, II, III, IV, V, VI, VII, VIII, IX, X, and XI, a moiety

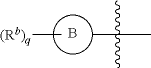

is selected from:

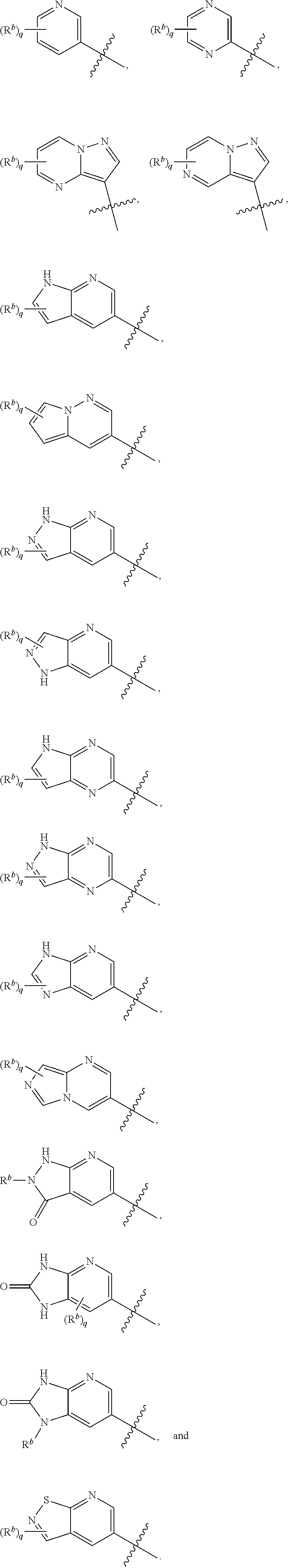

In some embodiments, a moiety

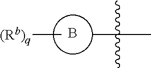

is selected from:

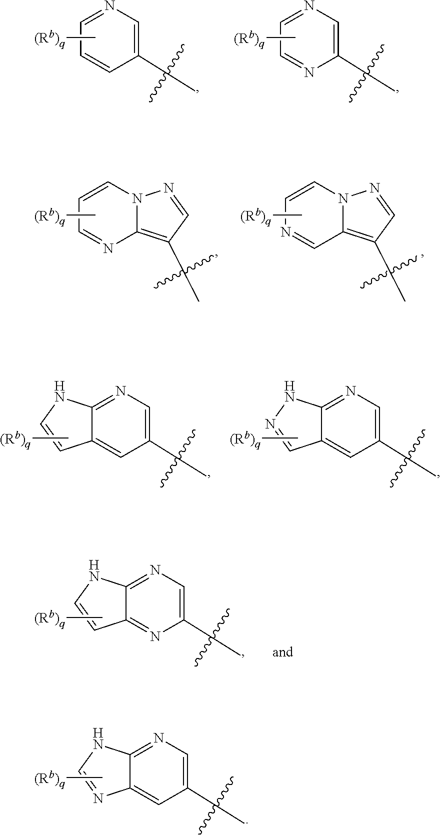

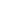

In some embodiments, a moiety

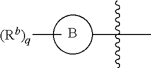

is selected from:

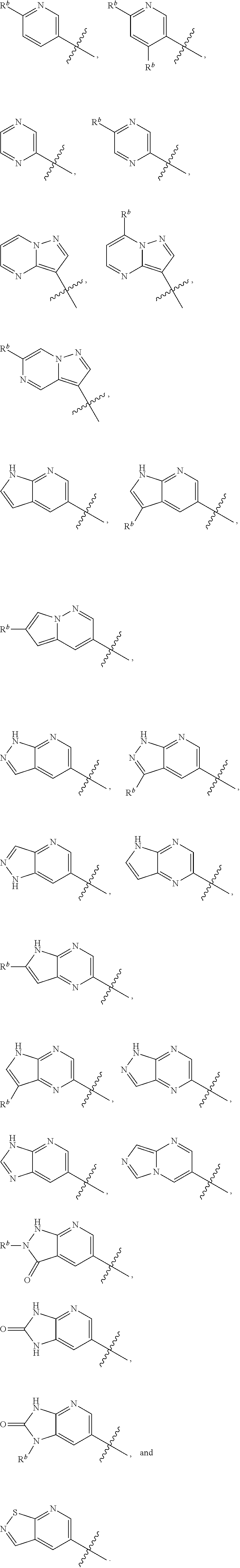

In some embodiments, a moiety

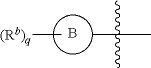

is selected from:

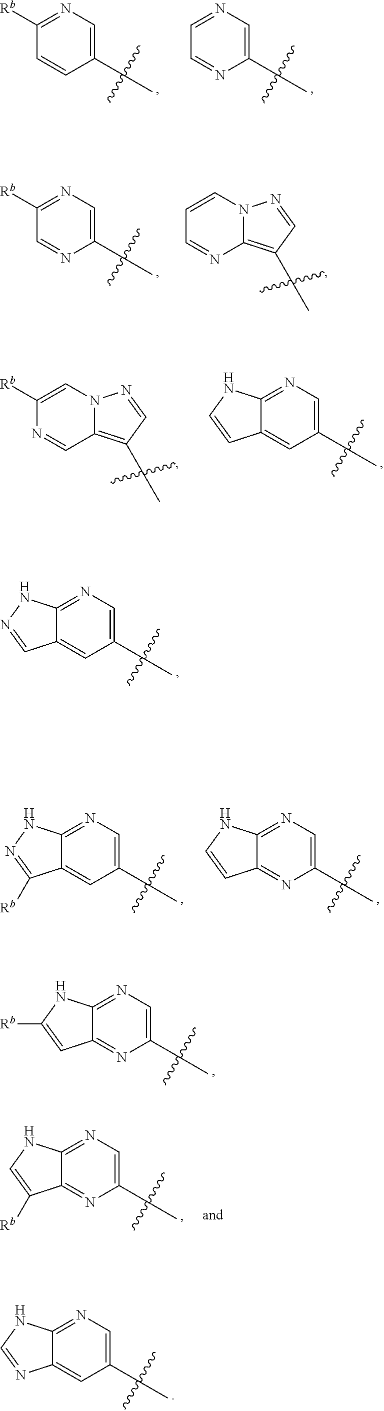

In some embodiments, a moiety

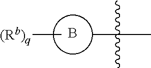

is selected from:

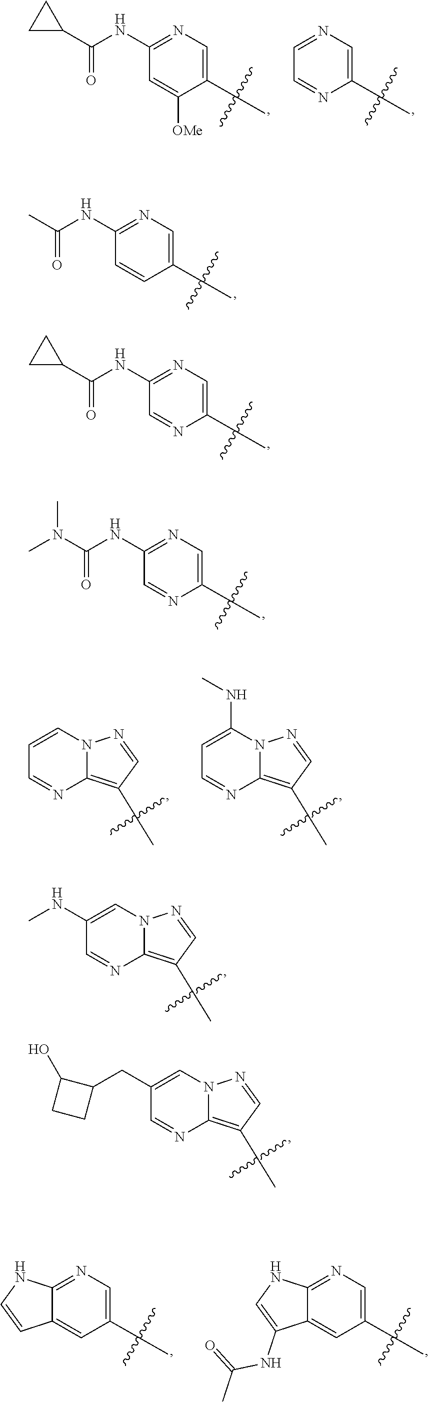

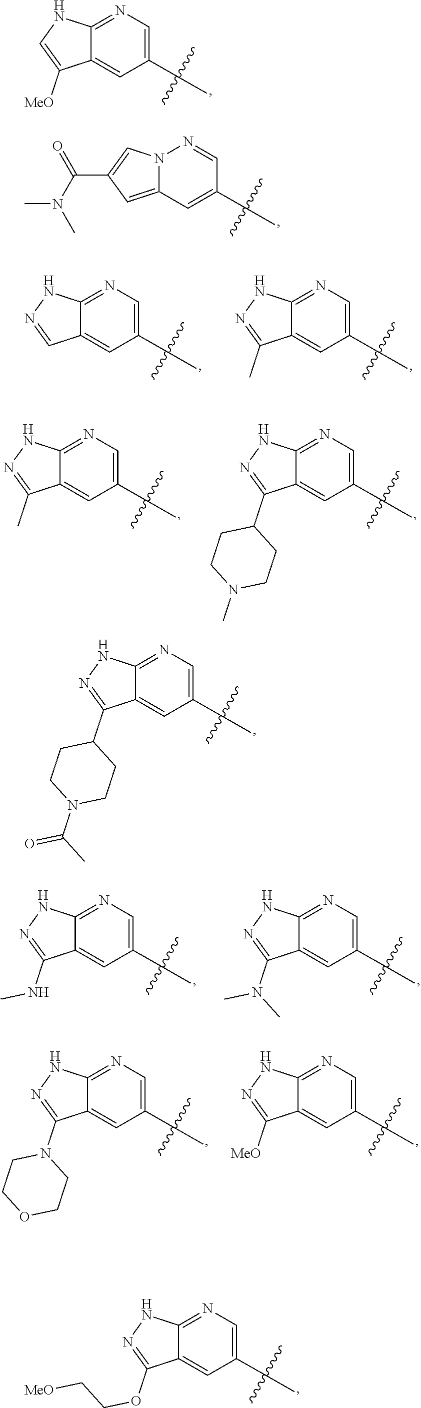

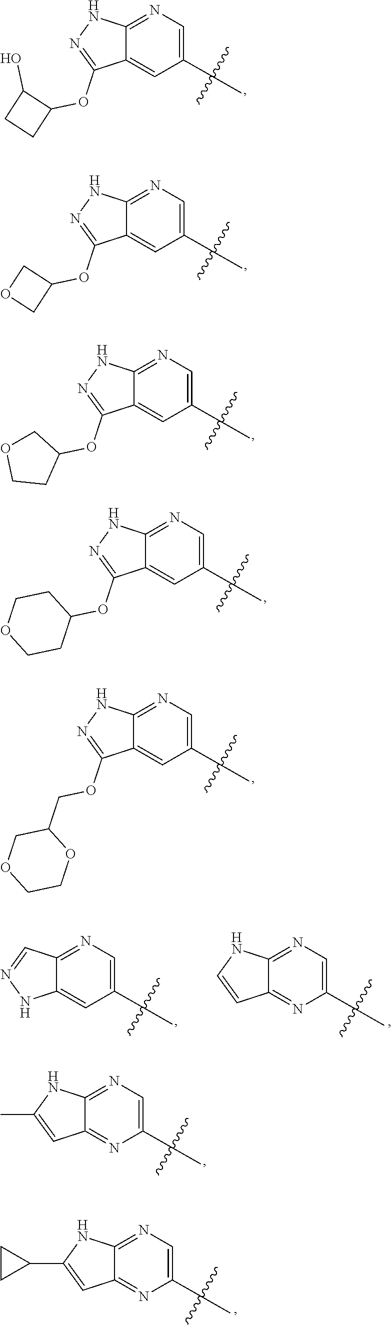

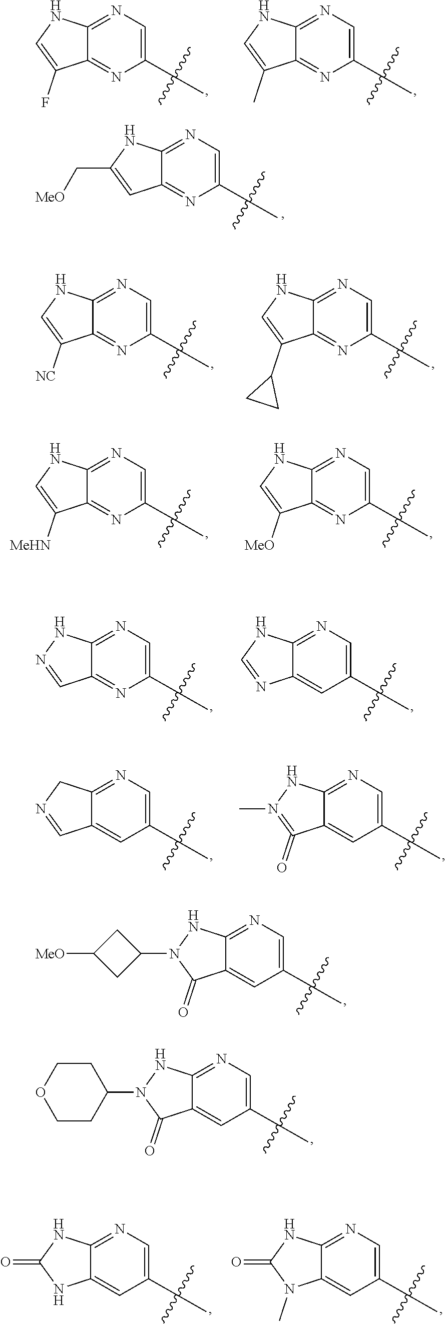

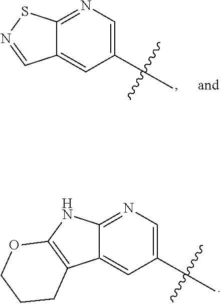 [SMILES: *c1cnc2sncc2c1 ; *c1cnc2[nH]c3c(c2c1)CCCO3]

In some embodiments, a moiety

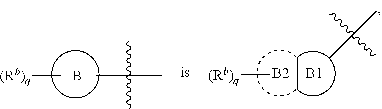

wherein:
Ring B1 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur.

It will be appreciated that Ring B1 and Ring B2, together, are substituted with q R^{b} groups. Accordingly, unless stated otherwise, substituents R^{b} may be attached to Ring B1, Ring B2, or both Ring B1 and Ring B2.

It will also be appreciated that at least one of Ring B1 and Ring B2 comprises a heteroatom, and that at least one of Ring B1 and Ring B2 is aromatic.

In some embodiments of any of Formulae A, B, D, I, IA, IB, IC, ID, II, IV, V, VI, VII, VIII, IX, X, and XI, Ring B1 is a 5- to 6-membered monocyclic heteroaryl having 1-3 heteroatoms independently selected from nitrogen, oxygen, and sulfur or a 5- to 6-membered saturated or partially unsaturated monocyclic heterocyclyl having 1-3 heteroatoms independently selected from nitrogen, oxygen, and sulfur.

In some embodiments, Ring B1 is phenyl. In some embodiments, when Ring B1 is phenyl, Ring B2 contains at least one heteroatom.

In some embodiments, Ring B1 is a 5- to 6-membered monocyclic heteroaryl having 1-3 heteroatoms independently selected from nitrogen, oxygen, and sulfur. In some embodiments, Ring B1 is a 5-membered monocyclic heteroaryl having 1-3 heteroatoms independently selected from nitrogen, oxygen, and sulfur. In some embodiments, Ring B1 is a pyrazole. In some embodiments, Ring B1 is a 6-membered monocyclic heteroaryl having 1-3 heteroatoms independently selected from nitrogen, oxygen, and sulfur. In some embodiments, Ring B1 is a pyridine, pyridazine, pyrimidine, or pyrazine. In some embodiments, Ring B1 is a pyridine or pyrazine.

In some embodiments, Ring B1 is a C₅₋₆ cycloaliphatic. In some embodiments, when Ring B1 is a C₅₋₆ cycloaliphatic, Ring B2 contains at least one heteroatom. In some embodiments, when Ring B1 is a C₅₋₆ cycloaliphatic, Ring B2 is aromatic.

In some embodiments, Ring B1 is a 5- to 6-membered saturated or partially unsaturated monocyclic heterocyclyl having 1-3 heteroatoms independently selected from nitrogen, oxygen, and sulfur. In some embodiments, when Ring B1 is a 5- to 6-membered saturated or partially unsaturated monocyclic heterocyclyl having 1-3 heteroatoms independently selected from nitrogen, oxygen, and sulfur, Ring B2 is aromatic. In some embodiments, Ring B1 is a 5-membered saturated or partially unsaturated monocyclic heterocyclyl having 1-3 heteroatoms independently selected from nitrogen, oxygen, and sulfur. In some embodiments, Ring B1 is a 6-membered saturated or partially unsaturated monocyclic heterocyclyl having 1-3 heteroatoms independently selected from nitrogen, oxygen, and sulfur.

In some embodiments, Ring B1 optionally fused to Ring B2 is selected from the group consisting of:

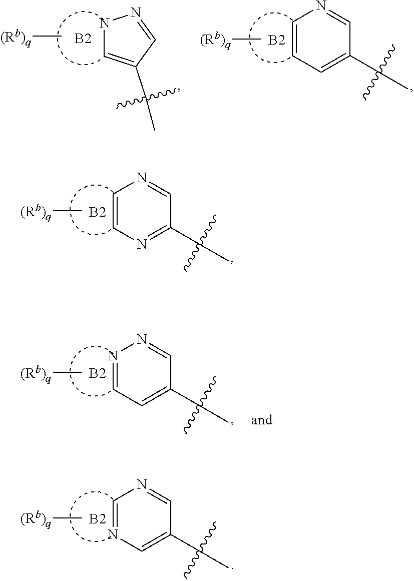

In some embodiments of any of Formulae A, B, D, I, IA, IB, IC, ID, II, IV, V, VI, VII, VIII, IX, X, and XI, Ring B2 is a 5- to 6-membered monocyclic heteroaryl having 1-3 heteroatoms independently selected from nitrogen, oxygen, and sulfur or a 5- to 6-membered saturated or partially unsaturated monocyclic heterocyclyl having 1-3 heteroatoms independently selected from nitrogen, oxygen, and sulfur.

In some embodiments, Ring B2 is absent.

In some embodiments, Ring B2 is phenyl. In some embodiments, when Ring B2 is phenyl, Ring B1 contains at least one heteroatom.

In some embodiments, Ring B2 is a 5- to 6-membered monocyclic heteroaryl having 1-3 heteroatoms independently selected from nitrogen, oxygen, and sulfur. In some embodiments, Ring B2 is a 5-membered monocyclic heteroaryl having 1-3 heteroatoms independently selected from nitrogen, oxygen, and sulfur. In some embodiments, Ring B2 is a pyrrole, pyrazole, imidazole, pyrazolidin-3-one, imidazolidin-2-one, or isothiazole. In some embodiments, Ring B2 is a pyrrole, pyrazole, or imidazole. In some embodiments, Ring B2 is a 6-membered monocyclic heteroaryl having 1-3 heteroatoms independently selected from nitrogen, oxygen, and sulfur. In some embodiments, Ring B1 is a pyrimidine or pyrazine.

In some embodiments, Ring B2 is a C₅₋₆ cycloaliphatic. In some embodiments, when Ring B2 is a C₅₋₆ cycloaliphatic, Ring B1 contains at least one heteroatom. In some embodiments, when Ring B2 is a C₅₋₆ cycloaliphatic, Ring B1 is aromatic.

In some embodiments, Ring B2 is a 5- to 6-membered saturated or partially unsaturated monocyclic heterocyclyl having 1-3 heteroatoms independently selected from nitrogen, oxygen, and sulfur. In some embodiments, when Ring B2 is a 5- to 6-membered saturated or partially unsaturated monocyclic heterocyclyl having 1-3 heteroatoms independently selected from nitrogen, oxygen, and sulfur, Ring B1 is aromatic. In some embodiments, Ring B2 is a 5-membered saturated or partially unsaturated monocyclic heterocyclyl having 1-3 heteroatoms independently selected from nitrogen, oxygen, and sulfur. In some embodiments, Ring B2 is a 6-membered saturated or partially unsaturated monocyclic heterocyclyl having 1-3 heteroatoms independently selected from nitrogen, oxygen, and sulfur.

In some embodiments, Ring B2, when present and fused to Ring B1, is selected from the group consisting of:

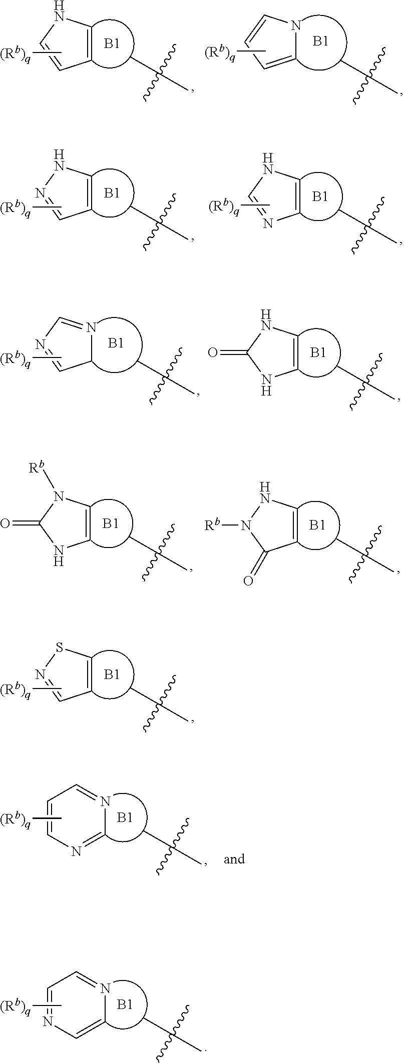

In some embodiments, Ring B1 and Ring B2 are both a 5- to 6-membered monocyclic heteroaryl having 1-3 heteroatoms independently selected from nitrogen, oxygen, and sulfur. In some embodiments, Ring B1 is a 5-membered saturated or partially unsaturated monocyclic heterocyclyl having 1-3 heteroatoms independently selected from nitrogen, oxygen, and sulfur; and Ring B2, when present, is a 6-membered monocyclic heteroaryl having 1-3 heteroatoms independently selected from nitrogen, oxygen, and sulfur. In some embodiments, Ring B1 is a 5-membered monocyclic heteroaryl having 1-3 heteroatoms independently selected from nitrogen, oxygen, and sulfur; Ring B1 is fused to Ring B2; and Ring B2 is a 6-membered monocyclic heteroaryl having 1-3 heteroatoms independently selected from nitrogen, oxygen, and sulfur. In some embodiments, Ring B1 is a 6-membered monocyclic heteroaryl having 1-3 heteroatoms independently selected from nitrogen, oxygen, and sulfur; and Ring B2, when present, is a 5-membered monocyclic heteroaryl having 1-3 heteroatoms independently selected from nitrogen, oxygen, and sulfur. In some embodiments, Ring B1 is a 6-membered monocyclic heteroaryl having 1-3 heteroatoms independently selected from nitrogen, oxygen, and sulfur; and Ring B2, when present, is a 5-membered saturated or partially unsaturated monocyclic heterocyclyl having 1-3 heteroatoms independently selected from nitrogen, oxygen, and sulfur. In some embodiments, (i) Ring B1 is a 5-membered monocyclic heteroaryl having 1-3 heteroatoms independently selected from nitrogen, oxygen, and sulfur; Ring B1 is fused to Ring B2; and Ring B2 is a 6-membered monocyclic heteroaryl having 1-3 heteroatoms independently selected from nitrogen, oxygen, and sulfur; or (ii) Ring B1 is a 6-membered monocyclic heteroaryl having 1-3 heteroatoms independently selected from nitrogen, oxygen, and sulfur; and Ring B2, when present, is a 5-membered monocyclic heteroaryl having 1-3 heteroatoms independently selected from nitrogen, oxygen, and sulfur.

In some embodiments, a moiety

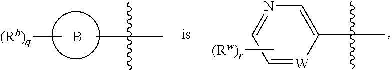

wherein:
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
two R^{w} groups, together with the atoms to which they are attached, combine to form an 5- to 6-membered partially unsaturated or aromatic ring substituted with q R^{b} groups; and
r is 0, 1, 2, or 3.

In some embodiments of any of Formulae A, C, D, E, I, IA, IB, IC, ID, III, IV, V, VI, VII, VIII, IX, X, and XI, W is CH. In some embodiments, W is CR^{w}. In some embodiments, W is N.

In some embodiments of any of Formulae A, C, D, E, I, IA, IB, IC, ID, III, IV, V, VI, VII, VIII, IX, X, and XI, each R^{w} is independently halogen, —CN, —N(R)₂, —C(O)N(R)₂, —N(R)C(O)R′, —N(R)C(O)OR, —N(R)C(O)N(R)₂, optionally substituted C₁₋₆ aliphatic, or optionally substituted C₃₋₆ cycloaliphatic. In some embodiments, each R^{w} is independently —N(R)C(O)R′, —N(R)C(O)N(R)₂, optionally substituted C₁₋₆ aliphatic, or optionally substituted C₃-6 cycloaliphatic. In some embodiments, each R^{w} is independently —OR, —N(R)C(O)R′, or —N(R)C(O)N(R)₂. In some embodiments, each R^{w} is independently —N(R)C(O)R′ or —N(R)C(O)N(R)₂.

In some embodiments, R^{w} is halogen. In some embodiments, R^{w} is fluoro. In some embodiments, R^{w} is chloro.

In some embodiments, R^{w} is —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, or —SO₃R′. In some embodiments, R^{w} is —CN, —N(R)₂, —C(O)N(R)₂, —N(R)C(O)R′, or —N(R)C(O)OR, —N(R)C(O)N(R)₂.

In some embodiments, R^{w} is —OR. In some embodiments, R^{w} is —OR, wherein R of R′ is optionally substituted C₁₋₆ aliphatic (e.g., optionally substituted C₁₋₆ alkyl). In some embodiments, R^{w} is —OCH₃.

In some embodiments, R′ is —N(R)C(O)R′. In some embodiments, R′ is —N(H)C(O)R′. In some embodiments, R′ is —N(H)C(O)(optionally substituted C₁₋₆ aliphatic). In some embodiments, R′ is —N(H)C(O)(C₁₋₆ alkyl). In some embodiments, R′ is —N(H)C(O)CH₃. In some embodiments, R^{w} is —N(H)C(O)(optionally substituted C₃₋₆ cycloaliphatic). In some embodiments, R^{w} is —N(H)C(O)(C₃₋₆ cycloalkyl). In some embodiments, R′ is —N(H)C(O)(cyclopropyl).

In some embodiments, R^{w} is —N(R)C(O)N(R)₂. In some embodiments, R^{w} is —N(H)C(O)N(R)₂. In some embodiments, R^{w} is —N(H)C(O)N(optionally substituted C₁₋₆ aliphatic)₂. In some embodiments, R^{w} is —N(H)C(O)N(C₁₋₆ alkyl)₂. In some embodiments, R′ is In some embodiments, R^{w} is —N(H)C(O)N(CH₃)₂.

In some embodiments, R′ is optionally substituted C₁₋₆ aliphatic. In some embodiments, R^{w} is optionally substituted straight-chain or branched C₁₋₆ aliphatic (i.e., optionally substituted acyclic C₁₋₆ aliphatic). In some embodiments, R^{w} is optionally substituted C₁₋₆ alkyl. In some embodiments, R^{w} is optionally substituted C₁₋₄ alkyl. In some embodiments, R′ is unsubstituted C₁₋₄ alkyl (e.g., methyl).

In some embodiments, R′ is optionally substituted C₃₋₆ cycloaliphatic. In some embodiments, R^{w} is optionally substituted C₃₋₆ cycloalkyl. In some embodiments, R^{w} is cyclopropyl. In some embodiments, R′ is cyclobutyl. In some embodiments, R^{w} is cyclopentyl. In some embodiments, R^{w} is cyclohexyl.

In some embodiments, R^{w} is optionally substituted 3- to 6-membered saturated or partially unsaturated monocyclic heterocyclyl having 1-3 heteroatoms independently selected from nitrogen, oxygen, and sulfur. In some embodiments, R′ is optionally substituted 3- to 6-membered saturated monocyclic heterocyclyl having 1-2 heteroatoms independently selected from nitrogen, oxygen, and sulfur.

In some embodiments, R^{w} is optionally substituted phenyl.

In some embodiments, R^{w} is optionally substituted 5- to 6-membered monocyclic heteroaryl having 1-4 heteroatoms independently selected from nitrogen, oxygen, and sulfur.

In some embodiments, two R^{w} groups, together with the atoms to which they are attached, combine to form a 5- to 6-membered partially unsaturated or aromatic ring substituted with q R^{b} groups. In some embodiments, two R^{w} groups, together with the atoms to which they are attached, combine to form a 5-membered partially unsaturated or aromatic ring substituted with q R^{b} groups. In some embodiments, two R^{w} groups, together with the atoms to which they are attached, combine to form a 5-membered aromatic ring (e.g., a pyrrole, pyrazole, imidazole, pyrazolidin-3-one, imidazolidin-2-one, or isothiazole) substituted with q R^{b} groups. In some embodiments, two R^{w} groups, together with the atoms to which they are attached, combine to form a 6-membered partially unsaturated or aromatic ring substituted with q R^{b} groups. In some embodiments, two R^{w} groups, together with the atoms to which they are attached, combine to form a 6-membered aromatic ring (e.g., a pyrimidine or pyrazine) substituted with q R^{b} groups.

In some embodiments any of Formulae A, C, D, E, I, IA, IB, IC, ID, III, IV, V, VI, VII, VIII, IX, X, and XI, r is 0, 1, or 2. In some embodiments, r is 0 or 1. In some embodiments,
r is 1 or 2. In some embodiments, r is 0. In some embodiments, r is 1. In some embodiments, r is 2. In some embodiments, r is 3. In some embodiments, q+r is no more than 5. In some embodiments, q+r is no more than 4.

In some embodiments, a moiety

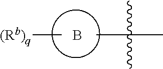

is:
(i)

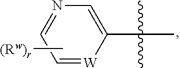

wherein W, R^{w}, and r are as defined above for Formula III and described in classes and subclasses herein, both singly and in combination; or
(ii)

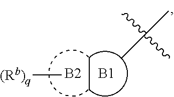

wherein Ring B2, R^{b}, and q are as defined above for Formula II and described in classes and subclasses herein, both singly and in combination; and:
Ring B1 is a 5-membered monocyclic heteroaryl having 1-3 heteroatoms independently selected from nitrogen, oxygen, and sulfur.

In some embodiments, a moiety

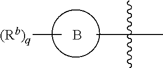

is:
(i)

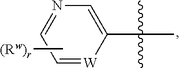

wherein W, R^{w}, and r are as defined above for Formula III and described in classes and subclasses herein, both singly and in combination; or
(ii)

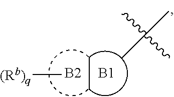

wherein Ring B2, R^{b} and q are as defined above for Formula II and described in classes and subclasses herein, both singly and in combination;
Ring B1 is a 5-membered monocyclic heteroaryl having 1-3 heteroatoms independently selected from nitrogen, oxygen, and sulfur; and
Ring B1 is fused to Ring B2.

In some embodiments, a moiety

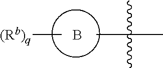

is:
(i)

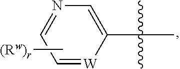

wherein W, R^{w}, and r are as defined above for Formula III and described in classes and subclasses herein, both singly and in combination; or
(ii)

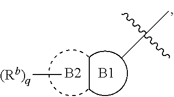

wherein R^{b} and q are as defined above for Formula II and described in classes and subclasses herein, both singly and in combination;
Ring B1 is a 5-membered monocyclic heteroaryl having 1-3 heteroatoms independently selected from nitrogen, oxygen, and sulfur;
Ring B1 is fused to Ring B2; and
Ring B2 is a 6-membered monocyclic heteroaryl having 1-3 heteroatoms independently selected from nitrogen, oxygen, and sulfur.

In some embodiments of any of Formulae A, B, C, D, E, I, IA, IB, IC, ID, II, III, IV, V, VI, VII, VIII, IX, X, and XI, each R is independently hydrogen, optionally substituted C₁₋₆ aliphatic, or optionally substituted 3- to 7-membered saturated or partially unsaturated monocyclic heterocyclyl having 1-3 heteroatoms independently selected from nitrogen, oxygen, and sulfur. In some embodiments, each R is independently hydrogen or optionally substituted C₁₋₆ aliphatic. In some embodiments, R is hydrogen. In some embodiments, R is optionally substituted C₁₋₆ aliphatic. In some embodiments, R is optionally substituted straight-chain or branched C₁₋₆ aliphatic (i.e., optionally substituted acyclic C₁₋₆ aliphatic). In some embodiments, R is optionally substituted C₁₋₆ alkyl (e.g., methyl, —CHF₂, —CH₂CH₂OCH₃, or —CH₂(1,4-dioxane)). In some embodiments, R is C₁₋₆ alkyl optionally substituted with one or more halogen (e.g., fluoro), —O(C₁₋₆ alkyl), or 3- to 7-membered saturated or partially unsaturated monocyclic heterocyclyl having 1-3 heteroatoms independently selected from nitrogen, oxygen, and sulfur. In some embodiments, R is optionally substituted C₃₋₇ cycloaliphatic. In some embodiments, R is optionally substituted C₃₋₇ cycloalkyl (e.g., cyclobutyl optionally substituted with one or more —OH). In some embodiments, R is optionally substituted 3- to 7-membered saturated or partially unsaturated monocyclic heterocyclyl having 1-3 heteroatoms independently selected from nitrogen, oxygen, and sulfur. In some embodiments, R is optionally substituted 4- to 6-membered saturated monocyclic heterocyclyl having 1-3 heteroatoms independently selected from nitrogen, oxygen, and sulfur (e.g., tetrahydrofuranyl or tetrahydropyranyl). In some embodiments, R is optionally substituted phenyl. In some embodiments, R is optionally substituted 5- to 6-membered monocyclic heteroaryl having 1-4 heteroatoms independently selected from nitrogen, oxygen, and sulfur. In some embodiments, two R when attached to the same nitrogen atom are taken together to form a 3- to 7-membered saturated or partially unsaturated monocyclic heterocyclyl having 0-1 additional heteroatoms independently selected from nitrogen, oxygen, and sulfur.

In some embodiments of any of Formulae A, B, C, D, E, I, IA, IB, IC, ID, II, III, IV, V, VI, VII, VIII, IX, X, and XI, each R′ is independently optionally substituted C₁₋₆ alkyl or optionally substituted C₃₋₇ cycloalkyl. In some embodiments, R′ is optionally substituted C₁₋₆ aliphatic. In some embodiments, R′ is optionally substituted straight-chain or branched C₁₋₆ aliphatic (i.e., optionally substituted acyclic C₁₋₆ aliphatic). In some embodiments, R′ is optionally substituted C₁₋₆ alkyl. In some embodiments, R′ is unsubstituted C₁₋₆ alkyl (e.g., methyl). In some embodiments, R′ is optionally substituted C₃₋₇ cycloaliphatic. In some embodiments, R′ is optionally substituted C₃₋₇ cycloalkyl (e.g., cyclopropyl).

In some embodiments of any of Formulae A, B, C, D, E, I, IA, IB, IC, ID, II, III, IV, V, VI, VII, VIII, IX, X, and XI, R⁷ is optionally substituted C₁₋₆ aliphatic; and when R⁸ is hydrogen, then p is 1, 2, 3, 4, or 5, as valency permits.

In some embodiments of any of Formulae A, B, C, D, E, I, IA, IB, IC, ID, II, III, IV, V, VI, VII, VIII, IX, X, and XI, R⁷ is optionally substituted C₁₋₆ aliphatic; Ring A is an optionally substituted group selected from phenyl and 5- to 6-membered monocyclic heteroaryl having 1-4 heteroatoms independently selected from nitrogen, oxygen, and sulfur; and when R′ is hydrogen, then p is 1, 2, 3, 4, or 5, as valency permits.

In some embodiments of any of Formulae A, B, C, D, E, I, IA, IB, IC, ID, II, III, IV, V, VI, VII, VIII, IX, X, and XI, R⁷ is optionally substituted C₁₋₆ aliphatic; R^{a} and R⁸ are not optionally substituted phenyl; and when R⁸ is hydrogen, then p is 1, 2, 3, 4, or 5, as valency permits.

In some embodiments of any of Formulae A, B, C, D, E, I, IA, IB, IC, ID, II, III, IV, and V the compound is not:

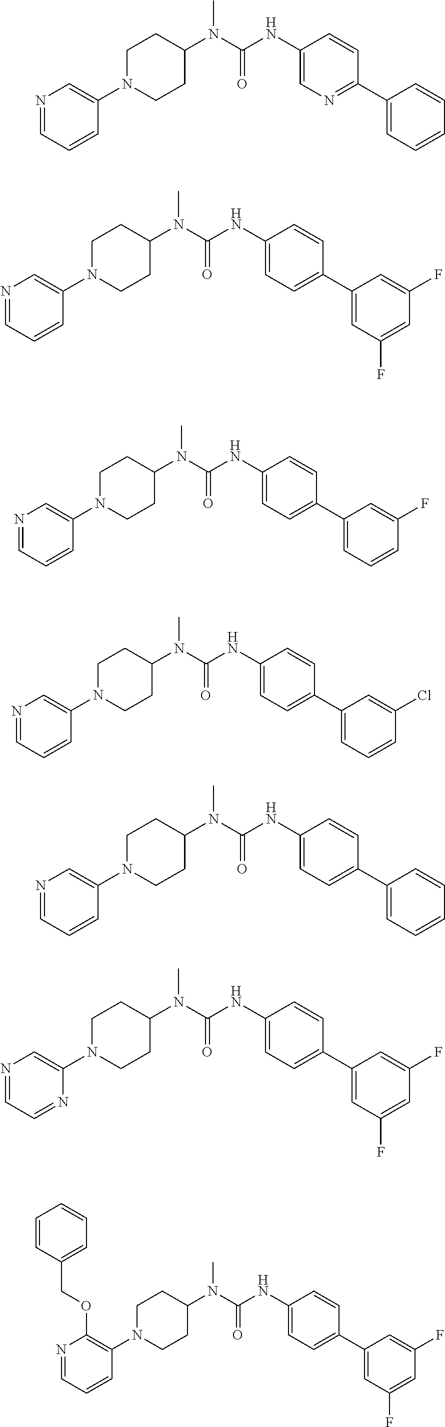

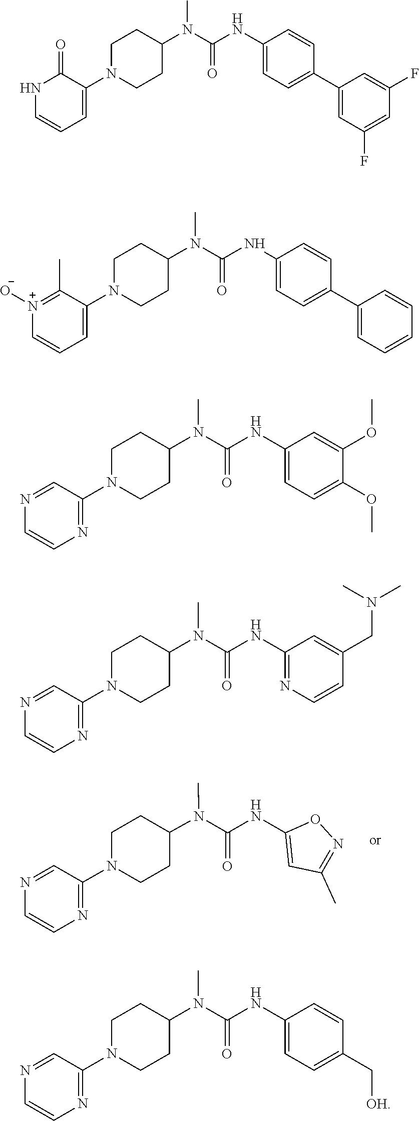 [SMILES: CN(C(=O)Nc1ccc(-c2cc(F)cc(F)c2)cc1)C1CCN(c2ccc[nH]c2=O)CC1 ; Cc1c(N2CCC(N(C)C(=O)Nc3ccc(-c4ccccc4)cc3)CC2)ccc[n+]1[O-] ; COc1ccc(NC(=O)N(C)C2CCN(c3cnccn3)CC2)cc1OC ; CN(C)Cc1ccnc(NC(=O)N(C)C2CCN(c3cnccn3)CC2)c1 ; Cc1cc(NC(=O)N(C)C2CCN(c3cnccn3)CC2)on1 ; CN(C(=O)Nc1ccc(CO)cc1)C1CCN(c2cnccn2)CC1]

In some embodiments of any of Formulae A, B, C, D, E, I, IA, IB, IC, ID, II, III, IV, V, VI, VII, VIII, IX, X, and XI, at least one of R^{a} or

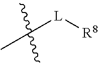

is C₁₋₄ haloalkyl (e.g., —CF₃).

In some embodiments of any of Formulae A, B, C, D, E, I, IA, IB, IC, ID, II, III, IV, and V, the compound is not:

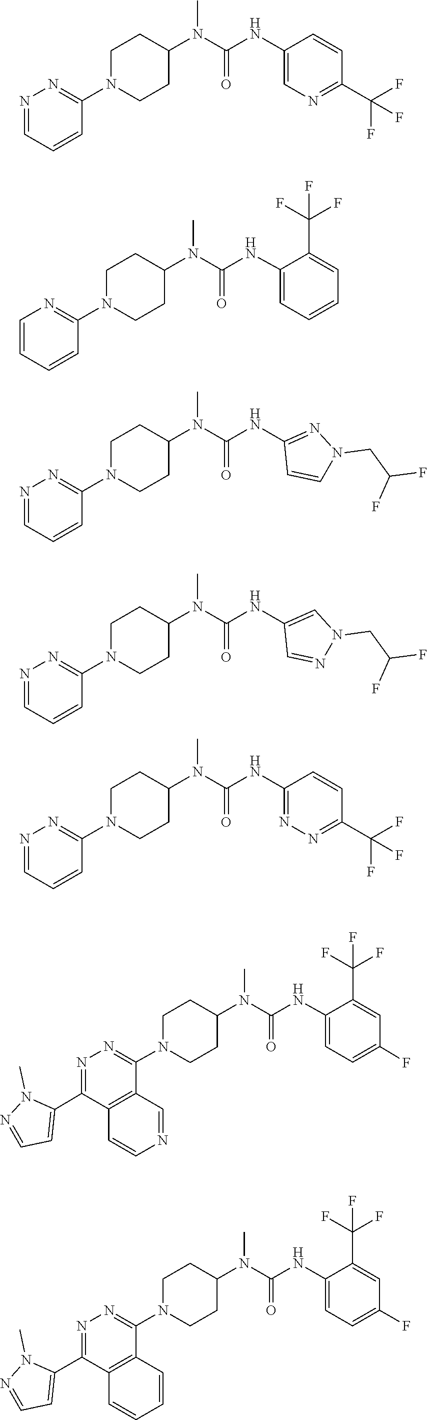

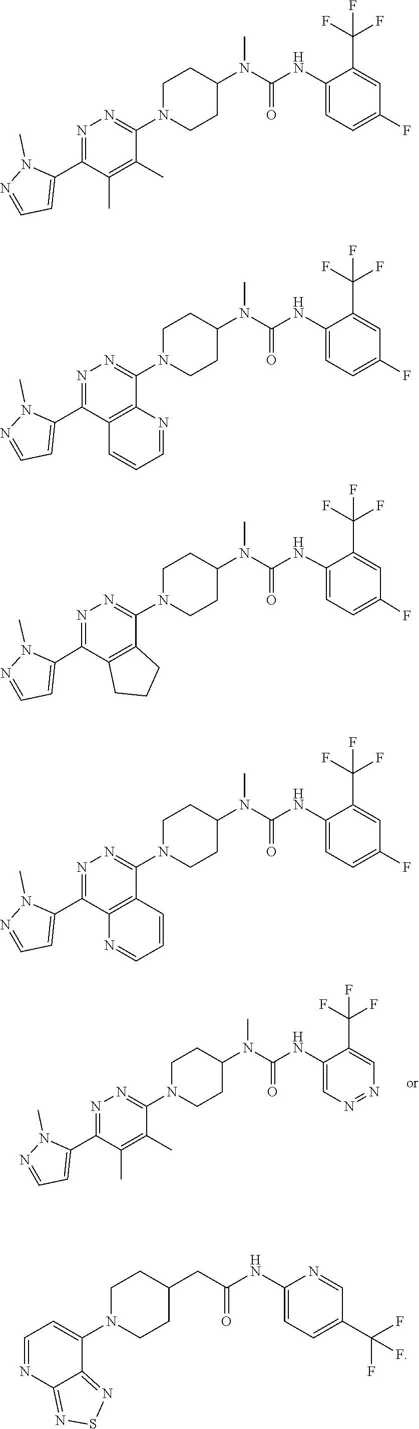 [SMILES: Cc1c(-c2ccnn2C)nnc(N2CCC(N(C)C(=O)Nc3ccc(F)cc3C(F)(F)F)CC2)c1C ; CN(C(=O)Nc1ccc(F)cc1C(F)(F)F)C1CCN(c2nnc(-c3ccnn3C)c3cccnc23)CC1 ; CN(C(=O)Nc1ccc(F)cc1C(F)(F)F)C1CCN(c2nnc(-c3ccnn3C)c3c2CCC3)CC1 ; CN(C(=O)Nc1ccc(F)cc1C(F)(F)F)C1CCN(c2nnc(-c3ccnn3C)c3ncccc23)CC1 ; Cc1c(-c2ccnn2C)nnc(N2CCC(N(C)C(=O)Nc3cnncc3C(F)(F)F)CC2)c1C ; O=C(CC1CCN(c2ccnc3nsnc23)CC1)Nc1ccc(C(F)(F)F)cn1]

In some embodiments of any of Formulae A, B, C, D, E, I, IA, IB, IC, ID, II, III, IV, V, VI, VII, VIII, IX, X, and XI, when a moiety

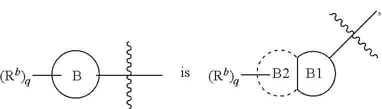

and Ring B1 is a 5-membered monocyclic heteroaryl having 1-3 heteroatoms independently selected from nitrogen, oxygen, and sulfur, then Ring B2 is present. In some such embodiments, Ring B2 is a 6-membered monocyclic heteroaryl having 1-3 heteroatoms independently selected from nitrogen, oxygen, and sulfur.

In some embodiments of any of Formulae A, B, C, D, E, I, IA, IB, IC, ID, II, III, IV, and V, the compound is not:

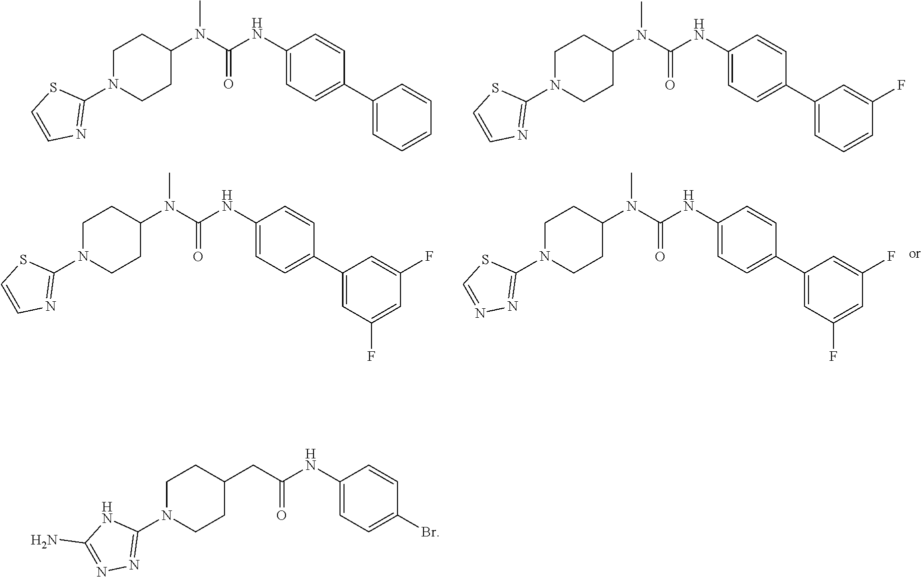

In some embodiments, the present disclosure provides a compound selected from Table 1, or a pharmaceutically acceptable salt thereof.

TABLE 1

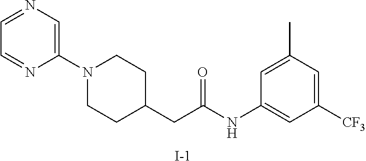 [SMILES: Cc1cc(NC(=O)CC2CCN(c3cnccn3)CC2)cc(C(F)(F)F)c1 ; [I-]]

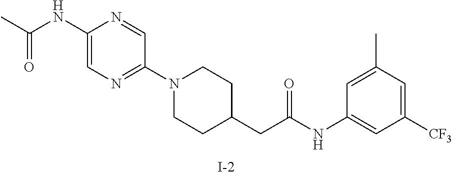

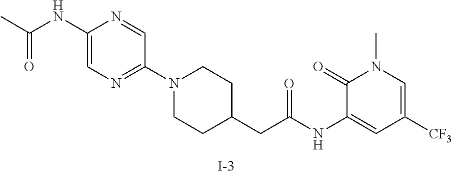

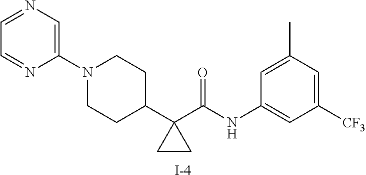

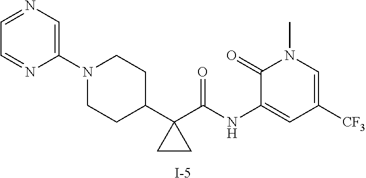

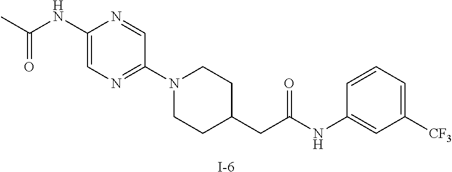

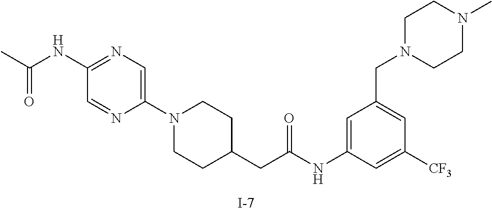

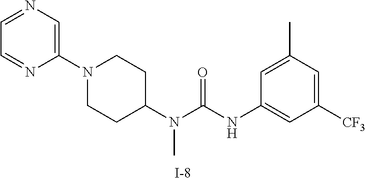

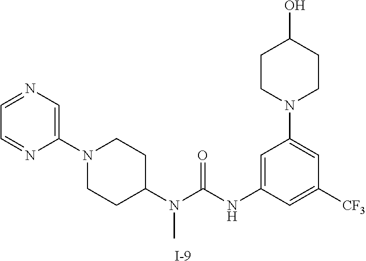

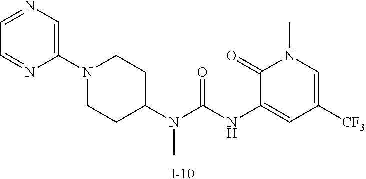

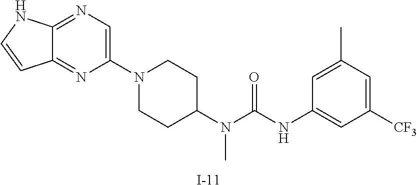

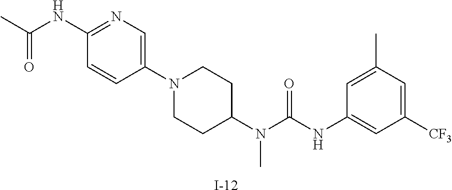

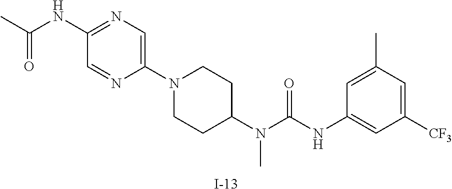

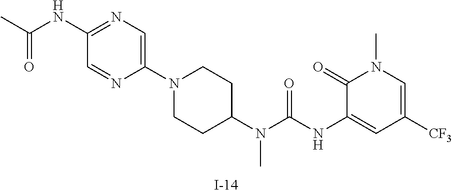

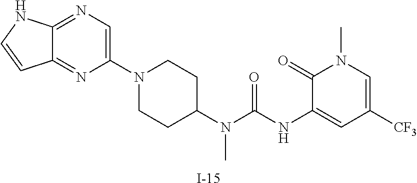

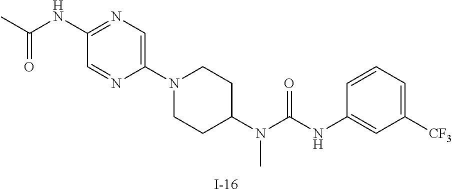

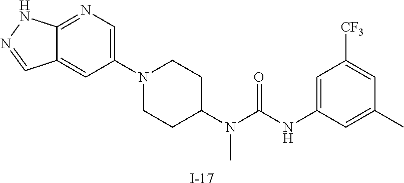

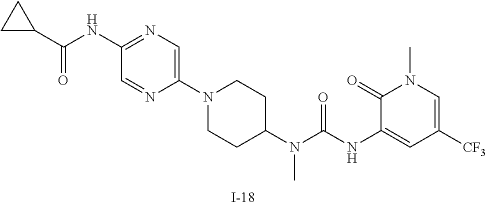

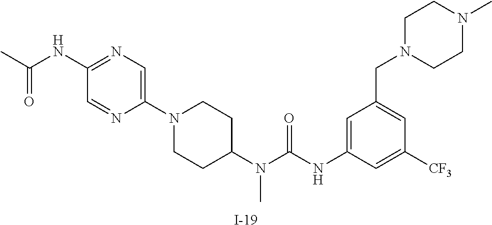

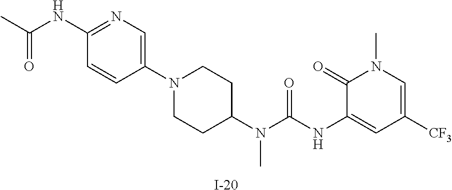

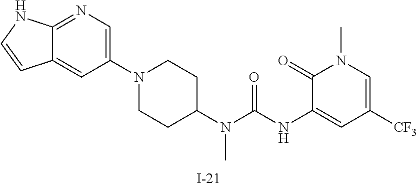

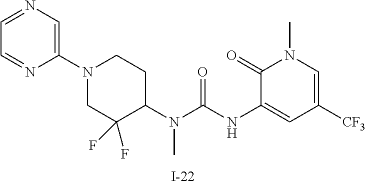

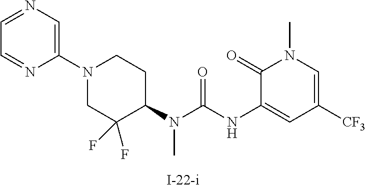 [SMILES: CN(C(=O)Nc1cc(C(F)(F)F)cn(C)c1=O)[C@@H]1CCN(c2cnccn2)CC1(F)F]

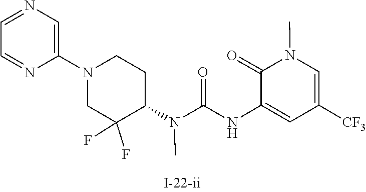 [SMILES: CN(C(=O)Nc1cc(C(F)(F)F)cn(C)c1=O)[C@H]1CCN(c2cnccn2)CC1(F)F]

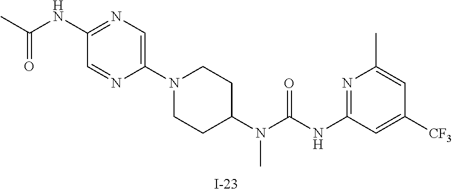

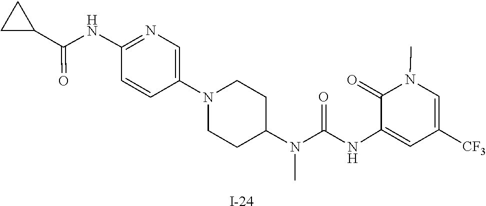

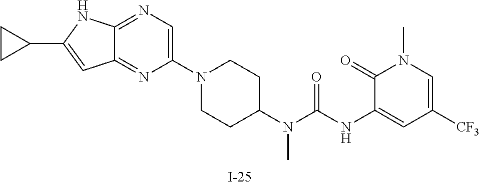

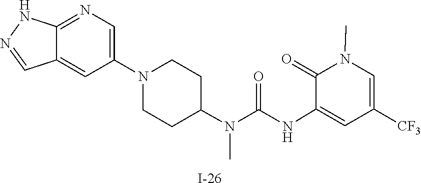

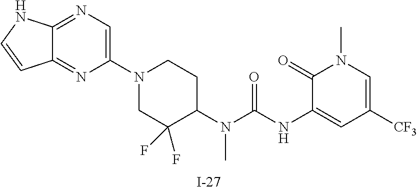

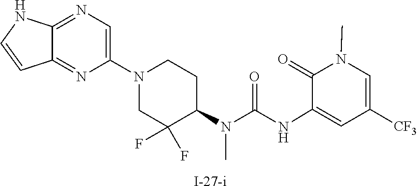

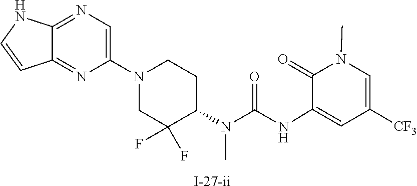

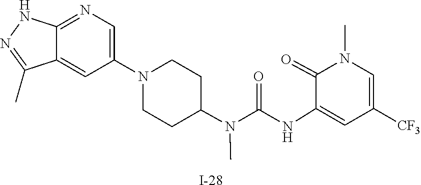

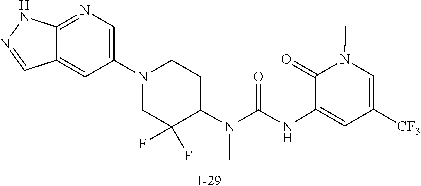

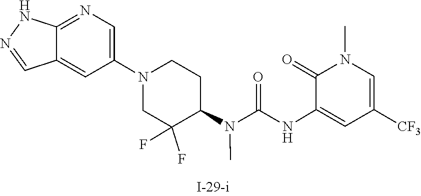 [SMILES: CN(C(=O)Nc1cc(C(F)(F)F)cn(C)c1=O)[C@@H]1CCN(c2cnc3[nH]ncc3c2)CC1(F)F]

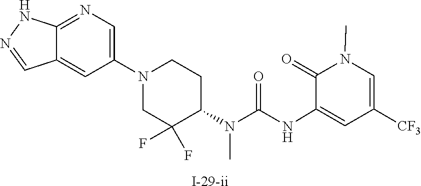

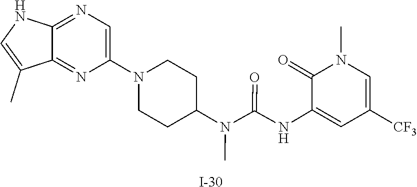

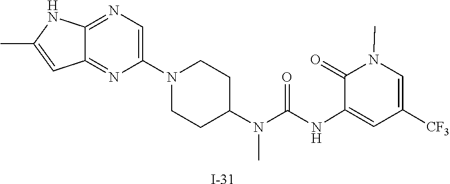

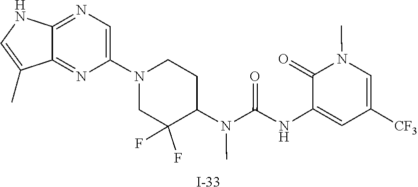

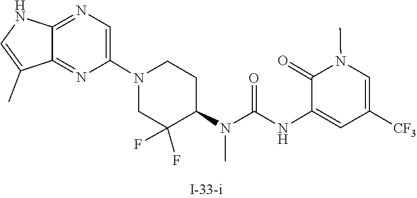 [SMILES: Cc1c[nH]c2ncc(N3CC[C@@H](N(C)C(=O)Nc4cc(C(F)(F)F)cn(C)c4=O)C(F)(F)C3)nc12]

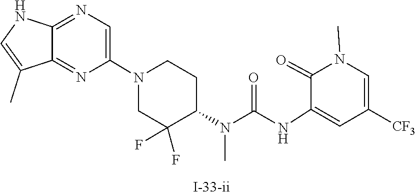 [SMILES: Cc1c[nH]c2ncc(N3CC[C@H](N(C)C(=O)Nc4cc(C(F)(F)F)cn(C)c4=O)C(F)(F)C3)nc12]

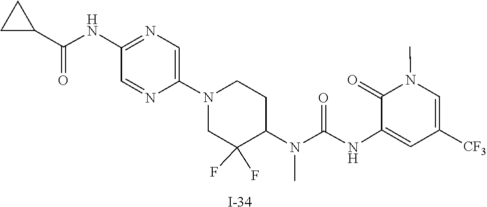

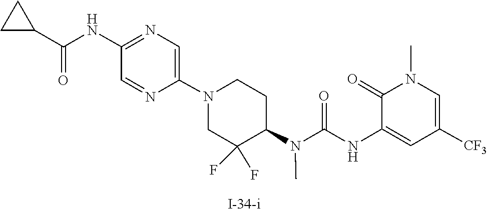 [SMILES: CN(C(=O)Nc1cc(C(F)(F)F)cn(C)c1=O)[C@@H]1CCN(c2cnc(NC(=O)C3CC3)cn2)CC1(F)F]

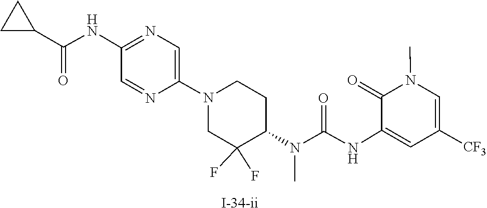

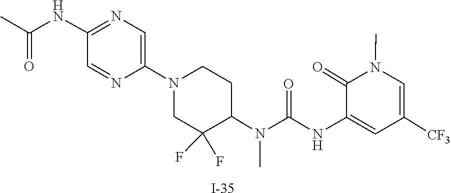

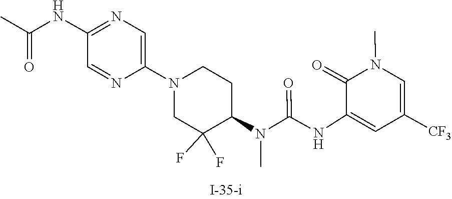 [SMILES: CC(=O)Nc1cnc(N2CC[C@@H](N(C)C(=O)Nc3cc(C(F)(F)F)cn(C)c3=O)C(F)(F)C2)cn1]

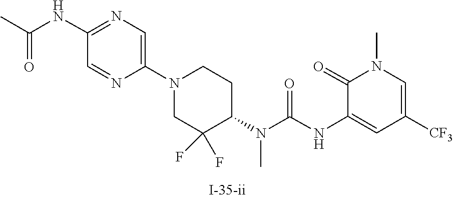 [SMILES: CC(=O)Nc1cnc(N2CC[C@H](N(C)C(=O)Nc3cc(C(F)(F)F)cn(C)c3=O)C(F)(F)C2)cn1]

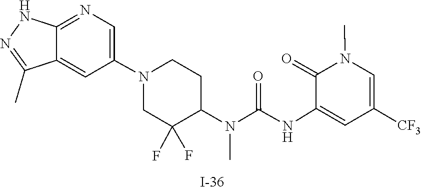

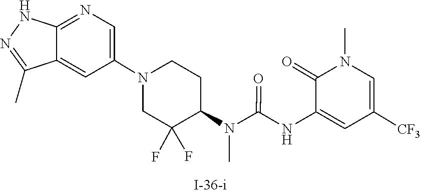 [SMILES: Cc1n[nH]c2ncc(N3CC[C@@H](N(C)C(=O)Nc4cc(C(F)(F)F)cn(C)c4=O)C(F)(F)C3)cc12]

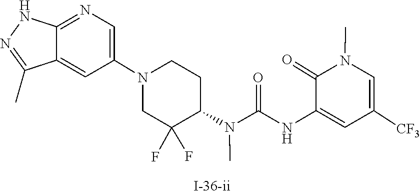 [SMILES: Cc1n[nH]c2ncc(N3CC[C@H](N(C)C(=O)Nc4cc(C(F)(F)F)cn(C)c4=O)C(F)(F)C3)cc12]

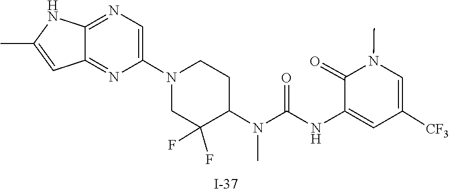

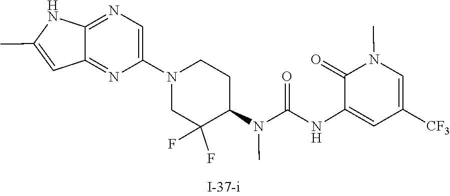 [SMILES: Cc1cc2nc(N3CC[C@@H](N(C)C(=O)Nc4cc(C(F)(F)F)cn(C)c4=O)C(F)(F)C3)cnc2[nH]1]

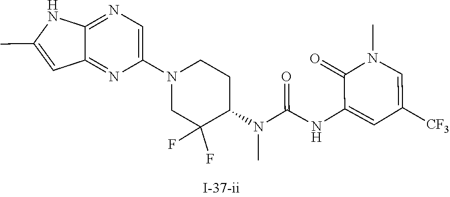 [SMILES: Cc1cc2nc(N3CC[C@H](N(C)C(=O)Nc4cc(C(F)(F)F)cn(C)c4=O)C(F)(F)C3)cnc2[nH]1]

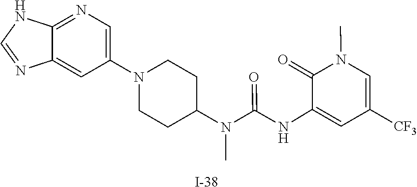

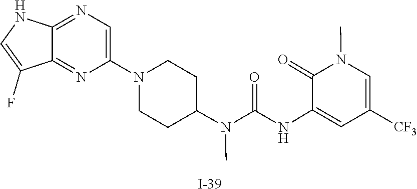

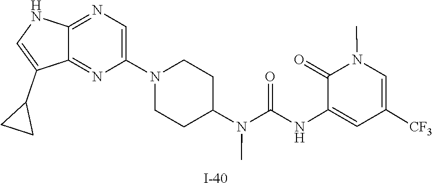

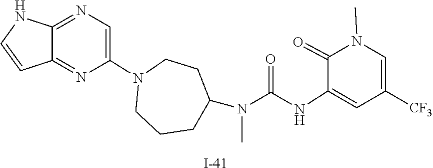

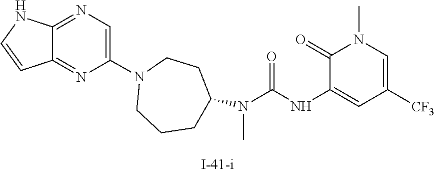 [SMILES: CN(C(=O)Nc1cc(C(F)(F)F)cn(C)c1=O)[C@@H]1CCCN(c2cnc3[nH]ccc3n2)CC1]

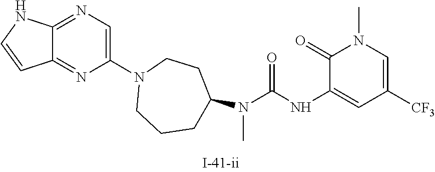 [SMILES: CN(C(=O)Nc1cc(C(F)(F)F)cn(C)c1=O)[C@H]1CCCN(c2cnc3[nH]ccc3n2)CC1]

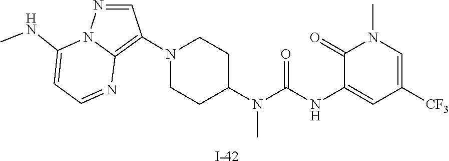

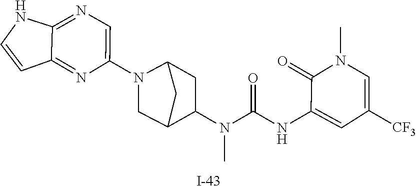

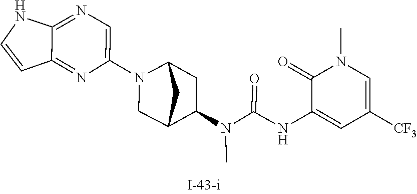 [SMILES: CN(C(=O)Nc1cc(C(F)(F)F)cn(C)c1=O)[C@@H]1C[C@@H]2C[C@H]1CN2c1cnc2[nH]ccc2n1]

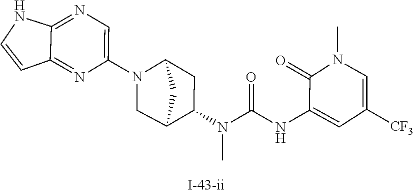 [SMILES: CN(C(=O)Nc1cc(C(F)(F)F)cn(C)c1=O)[C@H]1C[C@H]2C[C@@H]1CN2c1cnc2[nH]ccc2n1]

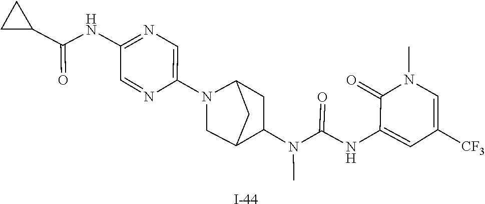

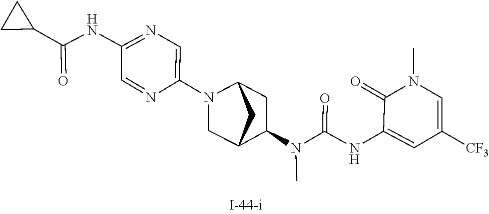 [SMILES: CN(C(=O)Nc1cc(C(F)(F)F)cn(C)c1=O)[C@@H]1C[C@@H]2C[C@H]1CN2c1cnc(NC(=O)C2CC2)cn1]

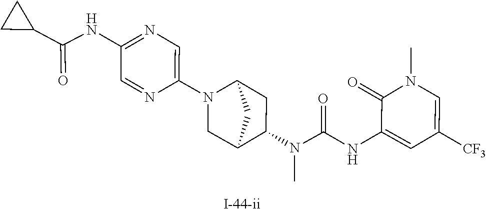 [SMILES: CN(C(=O)Nc1cc(C(F)(F)F)cn(C)c1=O)[C@H]1C[C@H]2C[C@@H]1CN2c1cnc(NC(=O)C2CC2)cn1]

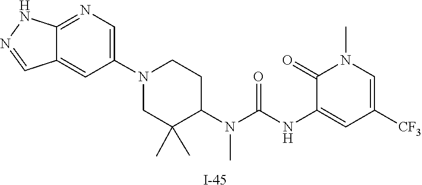

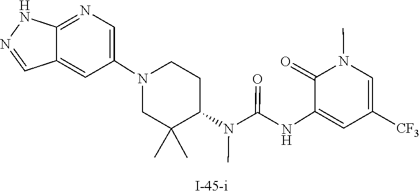 [SMILES: CN(C(=O)Nc1cc(C(F)(F)F)cn(C)c1=O)[C@H]1CCN(c2cnc3[nH]ncc3c2)CC1(C)C]

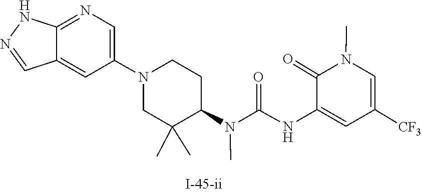 [SMILES: CN(C(=O)Nc1cc(C(F)(F)F)cn(C)c1=O)[C@@H]1CCN(c2cnc3[nH]ncc3c2)CC1(C)C]

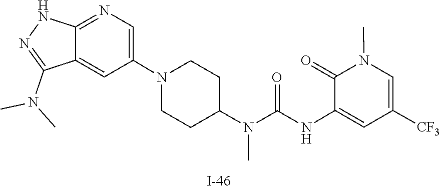

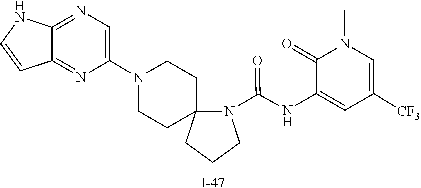

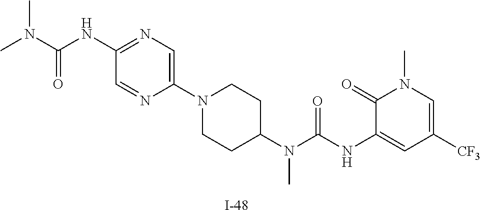

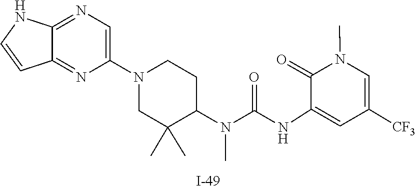

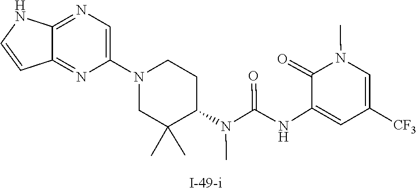 [SMILES: CN(C(=O)Nc1cc(C(F)(F)F)cn(C)c1=O)[C@H]1CCN(c2cnc3[nH]ccc3n2)CC1(C)C]

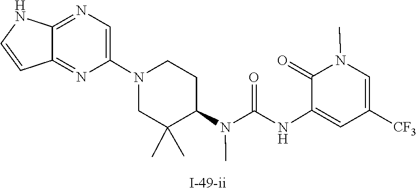 [SMILES: CN(C(=O)Nc1cc(C(F)(F)F)cn(C)c1=O)[C@@H]1CCN(c2cnc3[nH]ccc3n2)CC1(C)C]

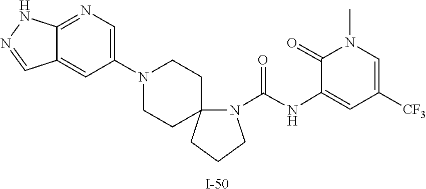

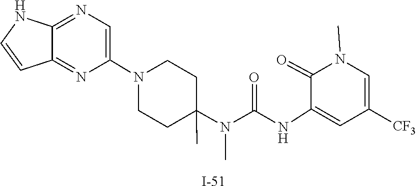

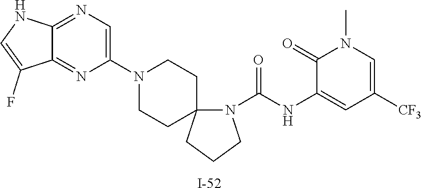

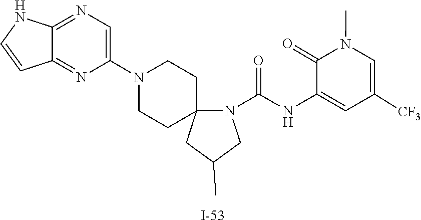

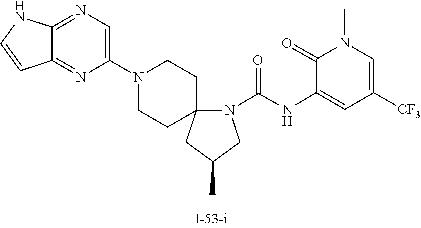 [SMILES: C[C@@H]1CN(C(=O)Nc2cc(C(F)(F)F)cn(C)c2=O)C2(CCN(c3cnc4[nH]ccc4n3)CC2)C1]

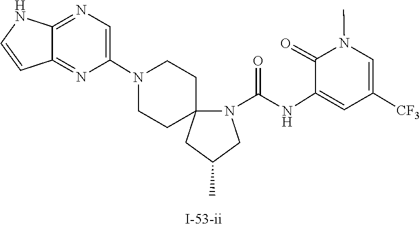 [SMILES: C[C@H]1CN(C(=O)Nc2cc(C(F)(F)F)cn(C)c2=O)C2(CCN(c3cnc4[nH]ccc4n3)CC2)C1]

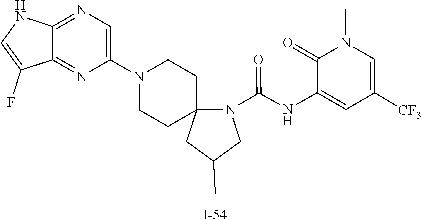

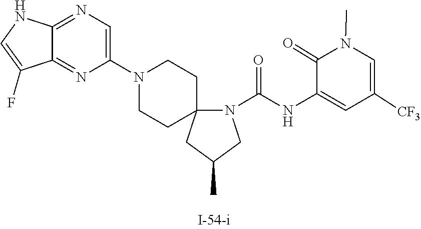 [SMILES: C[C@@H]1CN(C(=O)Nc2cc(C(F)(F)F)cn(C)c2=O)C2(CCN(c3cnc4[nH]cc(F)c4n3)CC2)C1]

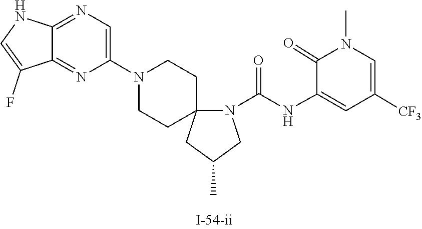 [SMILES: C[C@H]1CN(C(=O)Nc2cc(C(F)(F)F)cn(C)c2=O)C2(CCN(c3cnc4[nH]cc(F)c4n3)CC2)C1]

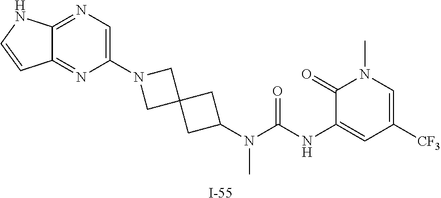

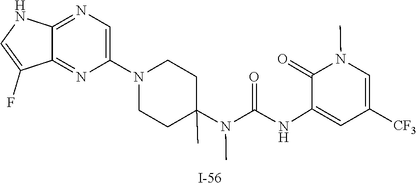

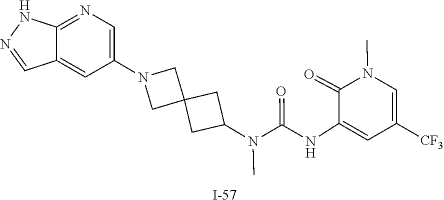

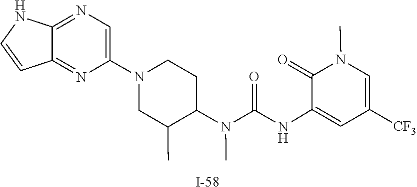

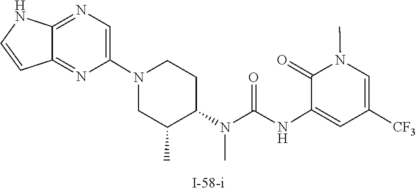 [SMILES: C[C@@H]1CN(c2cnc3[nH]ccc3n2)CC[C@@H]1N(C)C(=O)Nc1cc(C(F)(F)F)cn(C)c1=O]

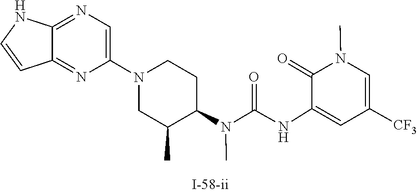 [SMILES: C[C@H]1CN(c2cnc3[nH]ccc3n2)CC[C@H]1N(C)C(=O)Nc1cc(C(F)(F)F)cn(C)c1=O]

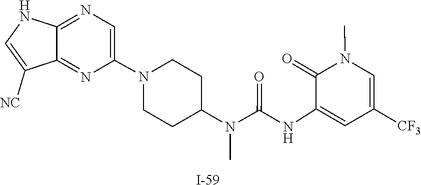

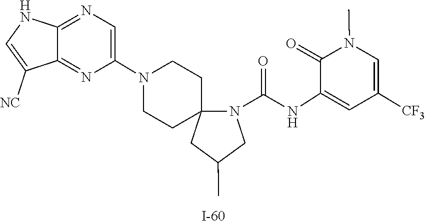

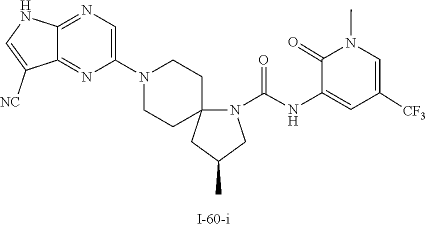 [SMILES: C[C@@H]1CN(C(=O)Nc2cc(C(F)(F)F)cn(C)c2=O)C2(CCN(c3cnc4[nH]cc(C#N)c4n3)CC2)C1]

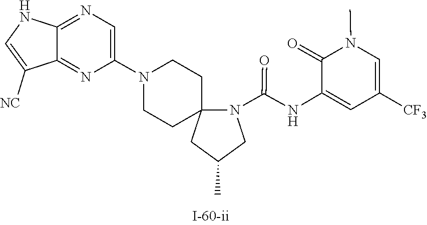 [SMILES: C[C@H]1CN(C(=O)Nc2cc(C(F)(F)F)cn(C)c2=O)C2(CCN(c3cnc4[nH]cc(C#N)c4n3)CC2)C1]

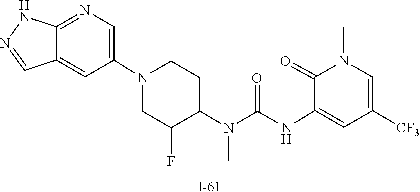

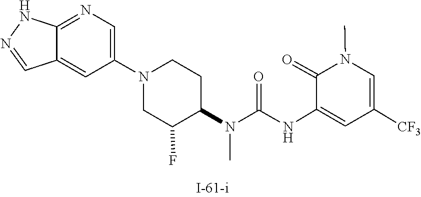 [SMILES: CN(C(=O)Nc1cc(C(F)(F)F)cn(C)c1=O)[C@@H]1CCN(c2cnc3[nH]ncc3c2)C[C@H]1F]

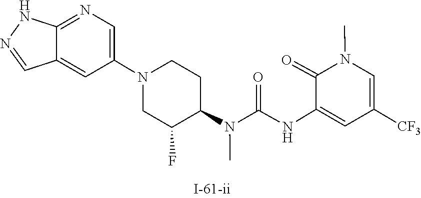 [SMILES: CN(C(=O)Nc1cc(C(F)(F)F)cn(C)c1=O)[C@@H]1CCN(c2cnc3[nH]ncc3c2)C[C@H]1F]

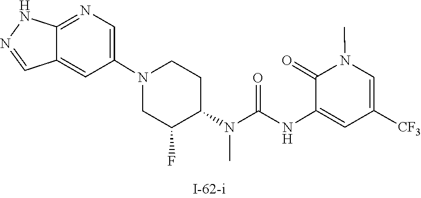 [SMILES: CN(C(=O)Nc1cc(C(F)(F)F)cn(C)c1=O)[C@H]1CCN(c2cnc3[nH]ncc3c2)C[C@H]1F]

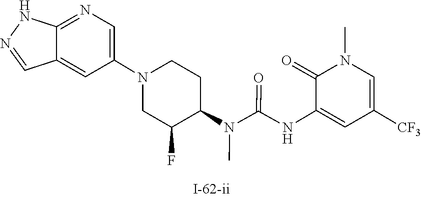 [SMILES: CN(C(=O)Nc1cc(C(F)(F)F)cn(C)c1=O)[C@@H]1CCN(c2cnc3[nH]ncc3c2)C[C@@H]1F]

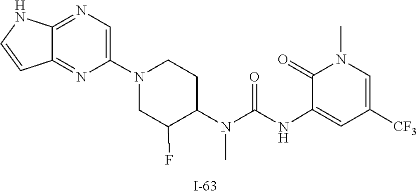

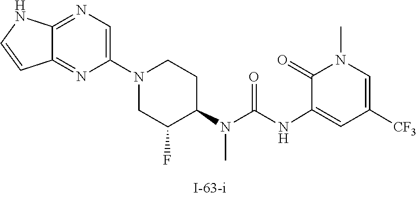 [SMILES: CN(C(=O)Nc1cc(C(F)(F)F)cn(C)c1=O)[C@@H]1CCN(c2cnc3[nH]ccc3n2)C[C@H]1F]

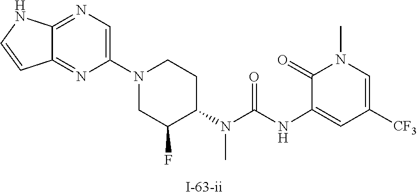 [SMILES: CN(C(=O)Nc1cc(C(F)(F)F)cn(C)c1=O)[C@H]1CCN(c2cnc3[nH]ccc3n2)C[C@@H]1F]

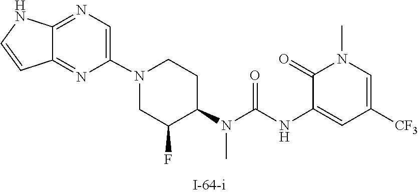 [SMILES: CN(C(=O)Nc1cc(C(F)(F)F)cn(C)c1=O)[C@@H]1CCN(c2cnc3[nH]ccc3n2)C[C@@H]1F]

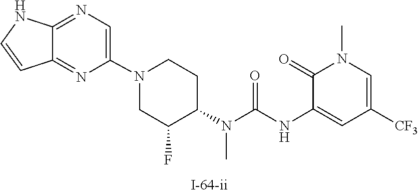 [SMILES: CN(C(=O)Nc1cc(C(F)(F)F)cn(C)c1=O)[C@H]1CCN(c2cnc3[nH]ccc3n2)C[C@H]1F]

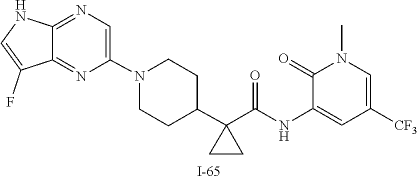

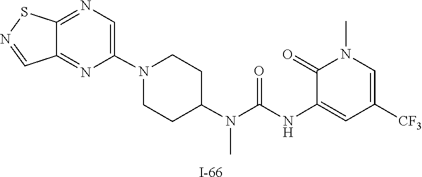

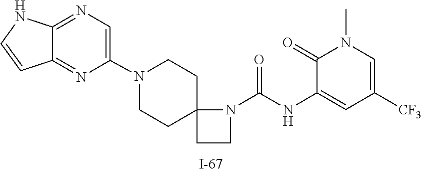

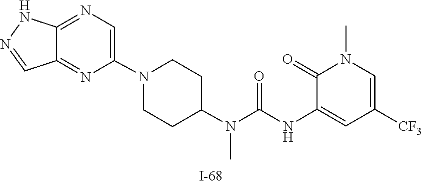

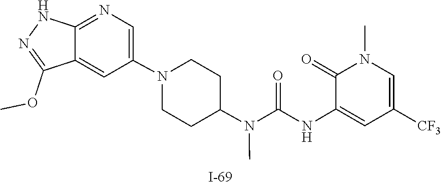

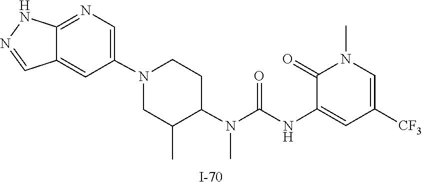

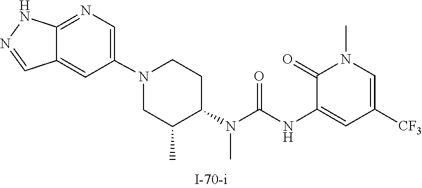 [SMILES: C[C@@H]1CN(c2cnc3[nH]ncc3c2)CC[C@@H]1N(C)C(=O)Nc1cc(C(F)(F)F)cn(C)c1=O]

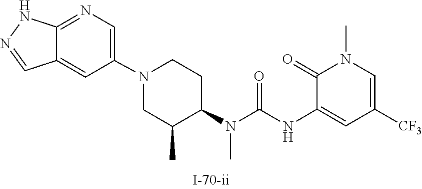 [SMILES: C[C@H]1CN(c2cnc3[nH]ncc3c2)CC[C@H]1N(C)C(=O)Nc1cc(C(F)(F)F)cn(C)c1=O]

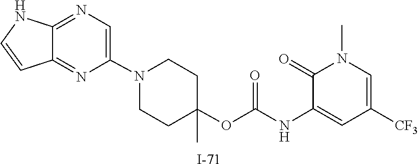

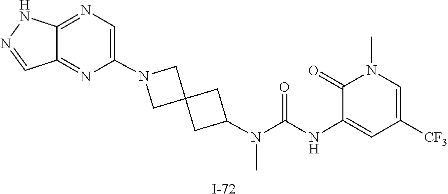

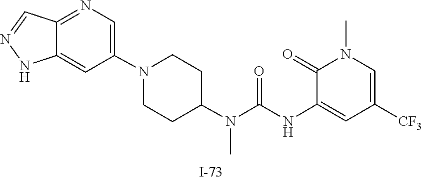

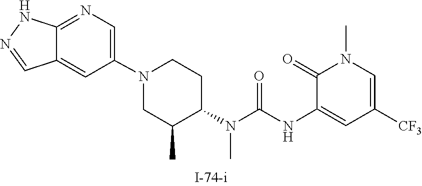 [SMILES: C[C@H]1CN(c2cnc3[nH]ncc3c2)CC[C@@H]1N(C)C(=O)Nc1cc(C(F)(F)F)cn(C)c1=O]

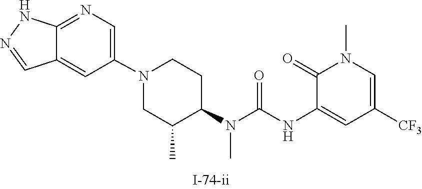 [SMILES: C[C@@H]1CN(c2cnc3[nH]ncc3c2)CC[C@H]1N(C)C(=O)Nc1cc(C(F)(F)F)cn(C)c1=O]

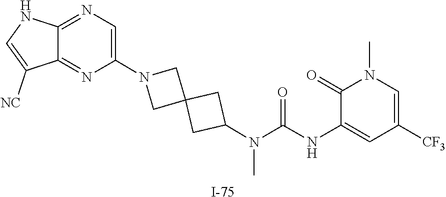

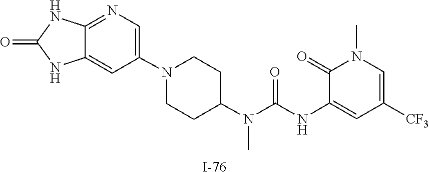

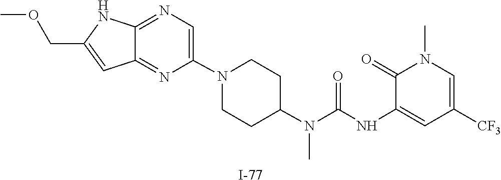

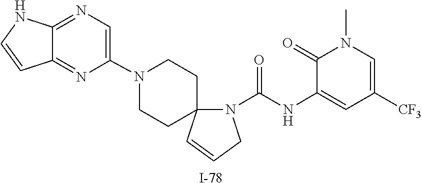

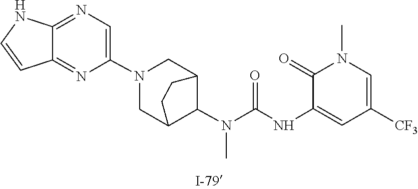 [SMILES: CN(C(=O)Nc1cc(C(F)(F)F)cn(C)c1=O)C1C2CCC1CN(c1cnc3[nH]ccc3n1)C2]

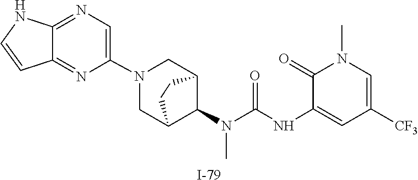

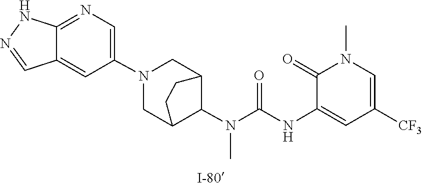 [SMILES: CN(C(=O)Nc1cc(C(F)(F)F)cn(C)c1=O)C1C2CCC1CN(c1cnc3[nH]ncc3c1)C2]

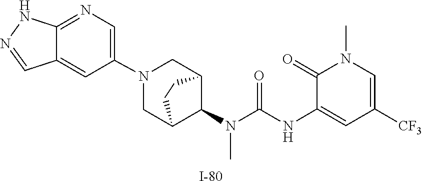

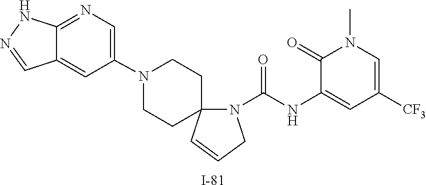

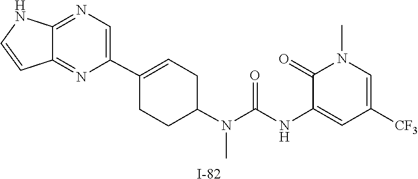

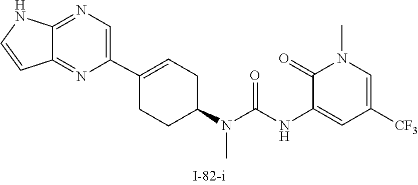 [SMILES: CN(C(=O)Nc1cc(C(F)(F)F)cn(C)c1=O)[C@@H]1CC=C(c2cnc3[nH]ccc3n2)CC1]

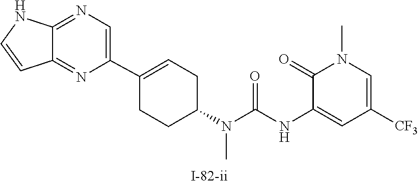 [SMILES: CN(C(=O)Nc1cc(C(F)(F)F)cn(C)c1=O)[C@H]1CC=C(c2cnc3[nH]ccc3n2)CC1]

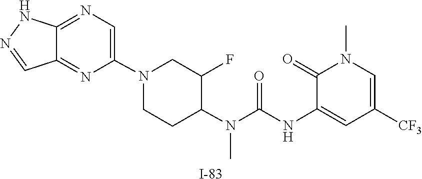

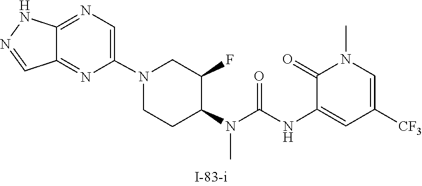 [SMILES: CN(C(=O)Nc1cc(C(F)(F)F)cn(C)c1=O)[C@H]1CCN(c2cnc3[nH]ncc3n2)C[C@H]1F]

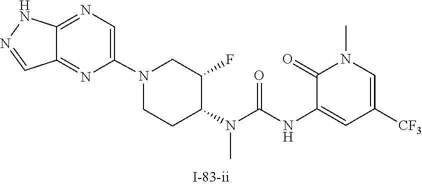 [SMILES: CN(C(=O)Nc1cc(C(F)(F)F)cn(C)c1=O)[C@@H]1CCN(c2cnc3[nH]ncc3n2)C[C@@H]1F]

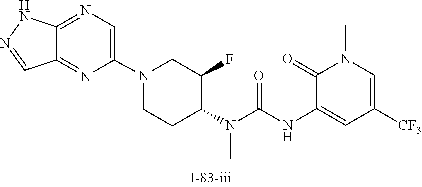 [SMILES: CN(C(=O)Nc1cc(C(F)(F)F)cn(C)c1=O)[C@@H]1CCN(c2cnc3[nH]ncc3n2)C[C@H]1F]

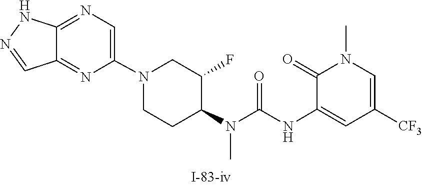 [SMILES: CN(C(=O)Nc1cc(C(F)(F)F)cn(C)c1=O)[C@H]1CCN(c2cnc3[nH]ncc3n2)C[C@@H]1F]

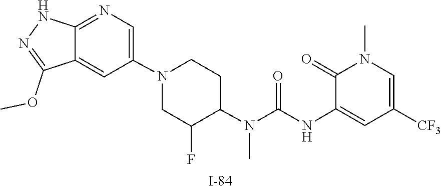

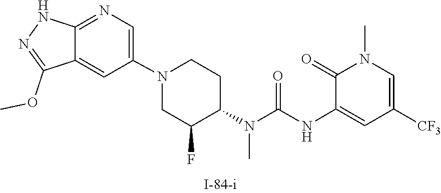 [SMILES: COc1n[nH]c2ncc(N3CC[C@H](N(C)C(=O)Nc4cc(C(F)(F)F)cn(C)c4=O)[C@@H](F)C3)cc12]

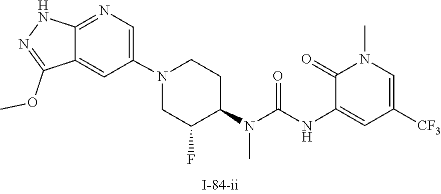 [SMILES: COc1n[nH]c2ncc(N3CC[C@@H](N(C)C(=O)Nc4cc(C(F)(F)F)cn(C)c4=O)[C@H](F)C3)cc12]

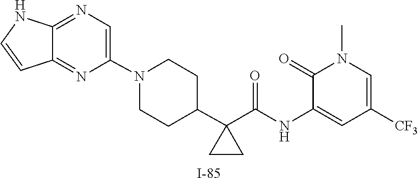

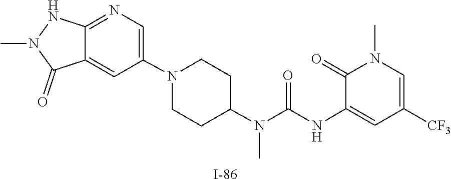

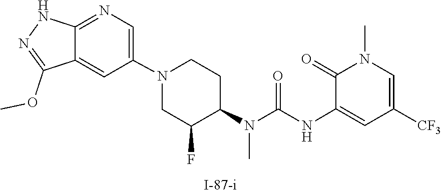 [SMILES: COc1n[nH]c2ncc(N3CC[C@@H](N(C)C(=O)Nc4cc(C(F)(F)F)cn(C)c4=O)[C@@H](F)C3)cc12]

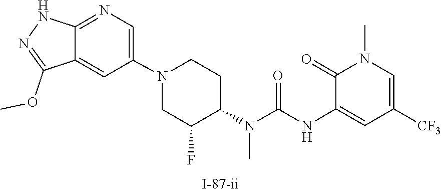 [SMILES: COc1n[nH]c2ncc(N3CC[C@H](N(C)C(=O)Nc4cc(C(F)(F)F)cn(C)c4=O)[C@H](F)C3)cc12]

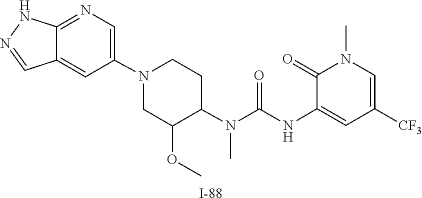

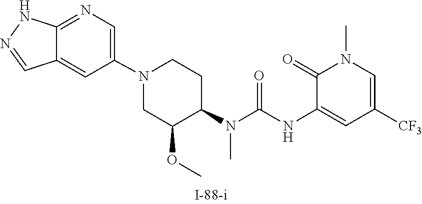 [SMILES: CO[C@H]1CN(c2cnc3[nH]ncc3c2)CC[C@H]1N(C)C(=O)Nc1cc(C(F)(F)F)cn(C)c1=O]

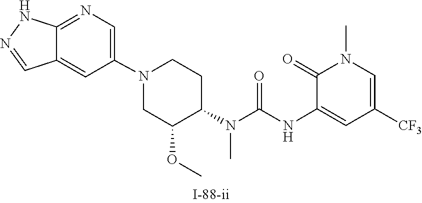 [SMILES: CO[C@@H]1CN(c2cnc3[nH]ncc3c2)CC[C@@H]1N(C)C(=O)Nc1cc(C(F)(F)F)cn(C)c1=O]

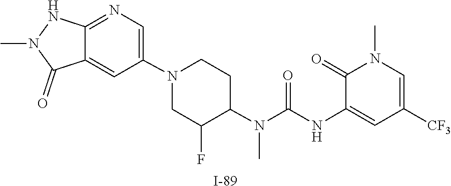

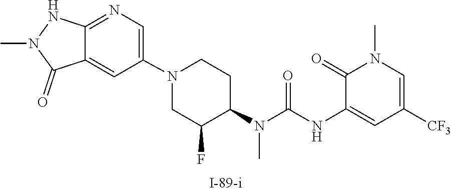 [SMILES: CN(C(=O)Nc1cc(C(F)(F)F)cn(C)c1=O)[C@@H]1CCN(c2cnc3[nH]n(C)c(=O)c3c2)C[C@@H]1F]

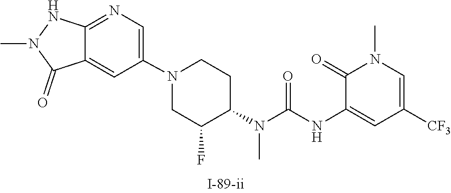 [SMILES: CN(C(=O)Nc1cc(C(F)(F)F)cn(C)c1=O)[C@H]1CCN(c2cnc3[nH]n(C)c(=O)c3c2)C[C@H]1F]

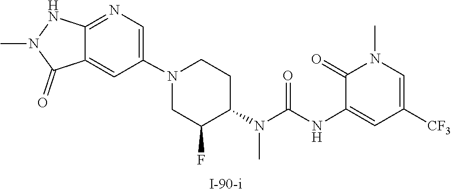 [SMILES: CN(C(=O)Nc1cc(C(F)(F)F)cn(C)c1=O)[C@H]1CCN(c2cnc3[nH]n(C)c(=O)c3c2)C[C@@H]1F]

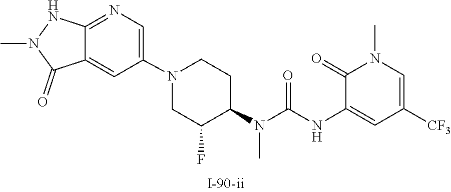 [SMILES: CN(C(=O)Nc1cc(C(F)(F)F)cn(C)c1=O)[C@@H]1CCN(c2cnc3[nH]n(C)c(=O)c3c2)C[C@H]1F]

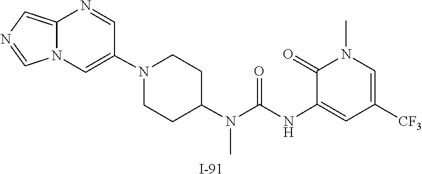

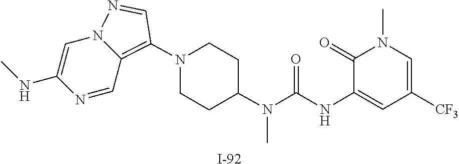

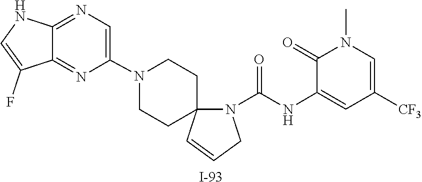

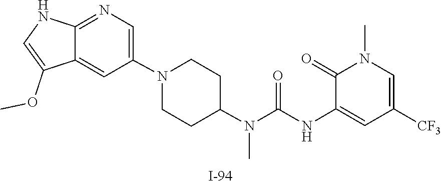

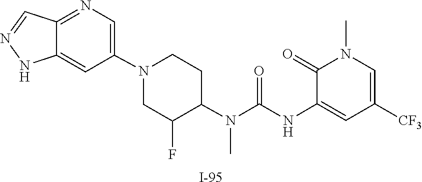

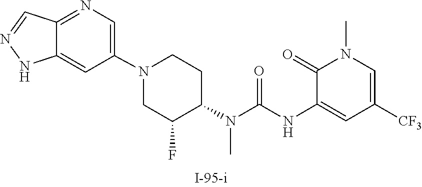 [SMILES: CN(C(=O)Nc1cc(C(F)(F)F)cn(C)c1=O)[C@H]1CCN(c2cnc3cn[nH]c3c2)C[C@H]1F]

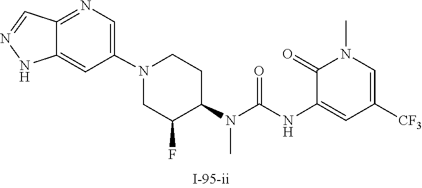 [SMILES: CN(C(=O)Nc1cc(C(F)(F)F)cn(C)c1=O)[C@@H]1CCN(c2cnc3cn[nH]c3c2)C[C@@H]1F]

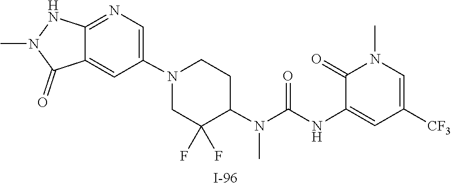

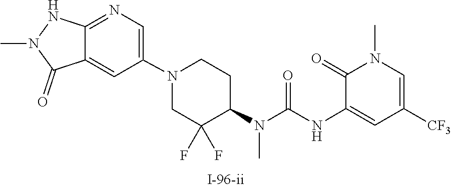 [SMILES: CN(C(=O)Nc1cc(C(F)(F)F)cn(C)c1=O)[C@@H]1CCN(c2cnc3[nH]n(C)c(=O)c3c2)CC1(F)F]

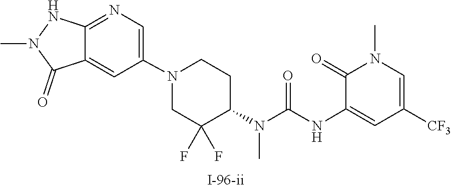 [SMILES: CN(C(=O)Nc1cc(C(F)(F)F)cn(C)c1=O)[C@H]1CCN(c2cnc3[nH]n(C)c(=O)c3c2)CC1(F)F]

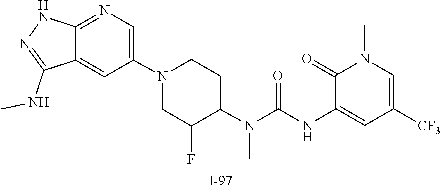

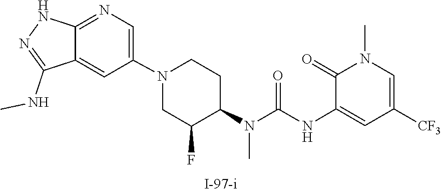 [SMILES: CNc1n[nH]c2ncc(N3CC[C@@H](N(C)C(=O)Nc4cc(C(F)(F)F)cn(C)c4=O)[C@@H](F)C3)cc12]

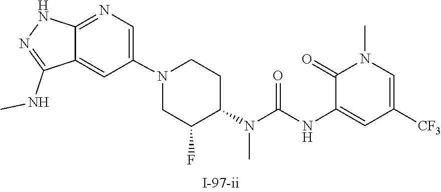 [SMILES: CNc1n[nH]c2ncc(N3CC[C@H](N(C)C(=O)Nc4cc(C(F)(F)F)cn(C)c4=O)[C@H](F)C3)cc12]

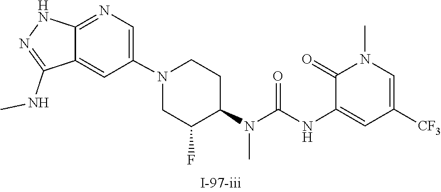 [SMILES: CNc1n[nH]c2ncc(N3CC[C@@H](N(C)C(=O)Nc4cc(C(F)(F)F)cn(C)c4=O)[C@H](F)C3)cc12]

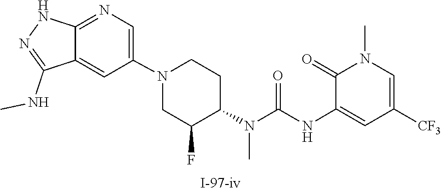 [SMILES: CNc1n[nH]c2ncc(N3CC[C@H](N(C)C(=O)Nc4cc(C(F)(F)F)cn(C)c4=O)[C@@H](F)C3)cc12]

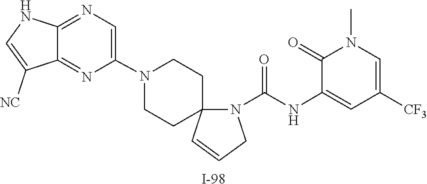

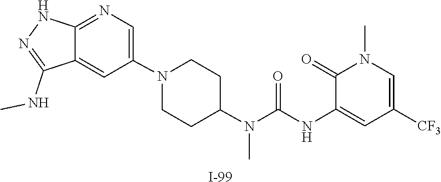

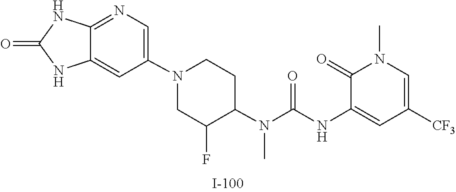 [SMILES: CN(C(=O)Nc1cc(C(F)(F)F)cn(C)c1=O)C1CCN(c2cnc3[nH]c(=O)[nH]c3c2)CC1F]

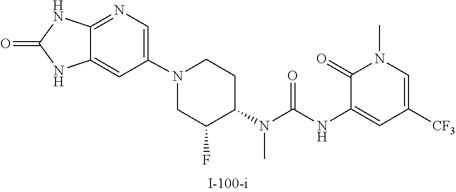 [SMILES: CN(C(=O)Nc1cc(C(F)(F)F)cn(C)c1=O)[C@H]1CCN(c2cnc3[nH]c(=O)[nH]c3c2)C[C@H]1F]

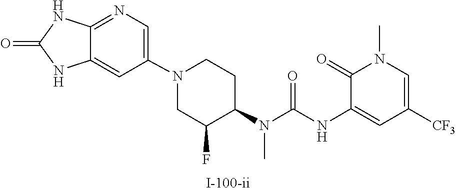 [SMILES: CN(C(=O)Nc1cc(C(F)(F)F)cn(C)c1=O)[C@@H]1CCN(c2cnc3[nH]c(=O)[nH]c3c2)C[C@@H]1F]

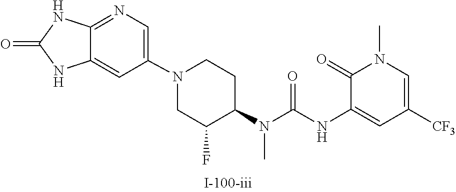 [SMILES: CN(C(=O)Nc1cc(C(F)(F)F)cn(C)c1=O)[C@@H]1CCN(c2cnc3[nH]c(=O)[nH]c3c2)C[C@H]1F]

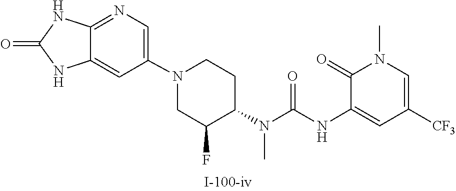 [SMILES: CN(C(=O)Nc1cc(C(F)(F)F)cn(C)c1=O)[C@H]1CCN(c2cnc3[nH]c(=O)[nH]c3c2)C[C@@H]1F]

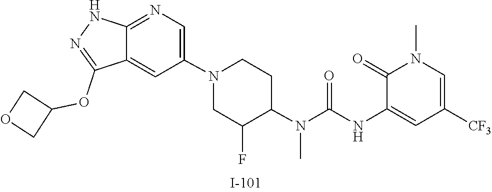 [SMILES: CN(C(=O)Nc1cc(C(F)(F)F)cn(C)c1=O)C1CCN(c2cnc3[nH]nc(OC4COC4)c3c2)CC1F]

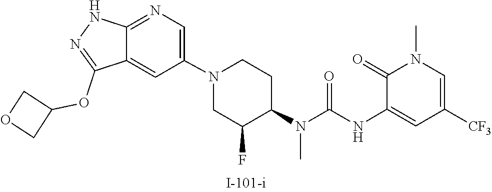 [SMILES: CN(C(=O)Nc1cc(C(F)(F)F)cn(C)c1=O)[C@@H]1CCN(c2cnc3[nH]nc(OC4COC4)c3c2)C[C@@H]1F]

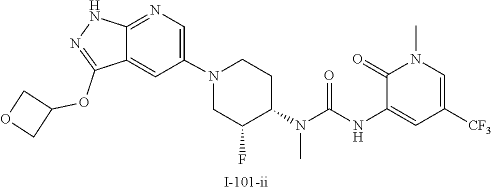 [SMILES: CN(C(=O)Nc1cc(C(F)(F)F)cn(C)c1=O)[C@H]1CCN(c2cnc3[nH]nc(OC4COC4)c3c2)C[C@H]1F]

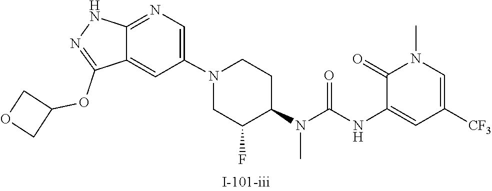 [SMILES: CN(C(=O)Nc1cc(C(F)(F)F)cn(C)c1=O)[C@@H]1CCN(c2cnc3[nH]nc(OC4COC4)c3c2)C[C@H]1F]

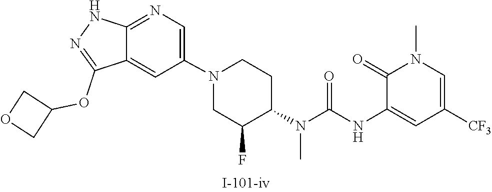 [SMILES: CN(C(=O)Nc1cc(C(F)(F)F)cn(C)c1=O)[C@H]1CCN(c2cnc3[nH]nc(OC4COC4)c3c2)C[C@@H]1F]

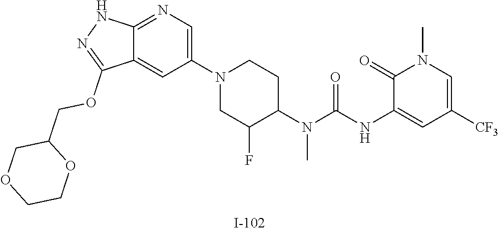 [SMILES: CN(C(=O)Nc1cc(C(F)(F)F)cn(C)c1=O)C1CCN(c2cnc3[nH]nc(OCC4COCCO4)c3c2)CC1F]

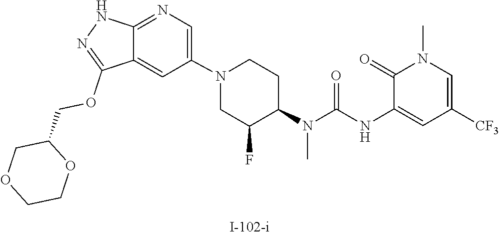 [SMILES: CN(C(=O)Nc1cc(C(F)(F)F)cn(C)c1=O)[C@@H]1CCN(c2cnc3[nH]nc(OC[C@H]4COCCO4)c3c2)C[C@@H]1F]

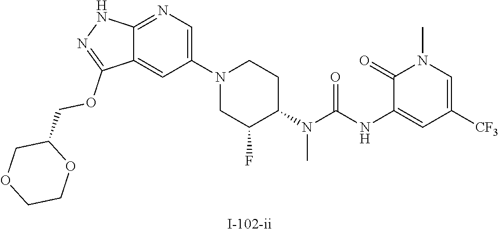 [SMILES: CN(C(=O)Nc1cc(C(F)(F)F)cn(C)c1=O)[C@H]1CCN(c2cnc3[nH]nc(OC[C@H]4COCCO4)c3c2)C[C@H]1F]

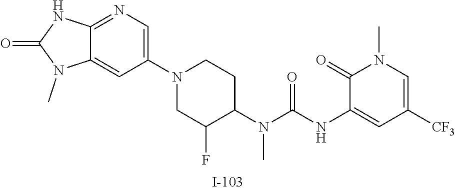 [SMILES: CN(C(=O)Nc1cc(C(F)(F)F)cn(C)c1=O)C1CCN(c2cnc3[nH]c(=O)n(C)c3c2)CC1F]

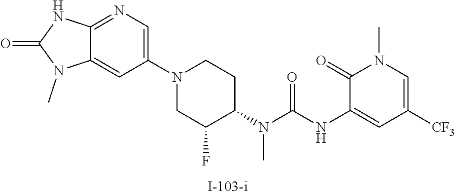 [SMILES: CN(C(=O)Nc1cc(C(F)(F)F)cn(C)c1=O)[C@H]1CCN(c2cnc3[nH]c(=O)n(C)c3c2)C[C@H]1F]

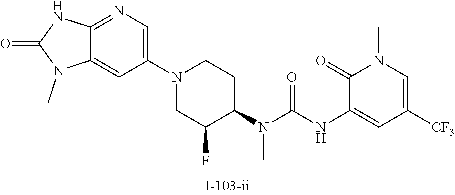 [SMILES: CN(C(=O)Nc1cc(C(F)(F)F)cn(C)c1=O)[C@@H]1CCN(c2cnc3[nH]c(=O)n(C)c3c2)C[C@@H]1F]

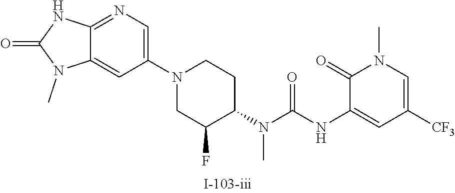 [SMILES: CN(C(=O)Nc1cc(C(F)(F)F)cn(C)c1=O)[C@H]1CCN(c2cnc3[nH]c(=O)n(C)c3c2)C[C@@H]1F]

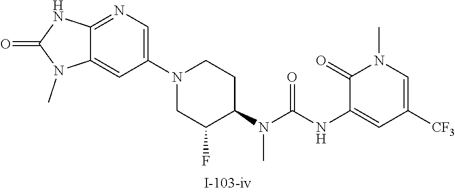 [SMILES: CN(C(=O)Nc1cc(C(F)(F)F)cn(C)c1=O)[C@@H]1CCN(c2cnc3[nH]c(=O)n(C)c3c2)C[C@H]1F]

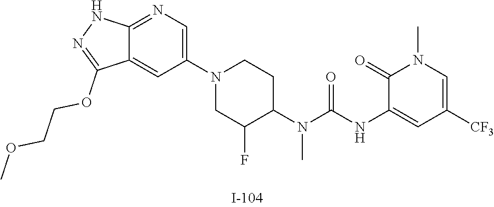 [SMILES: COCCOc1n[nH]c2ncc(N3CCC(N(C)C(=O)Nc4cc(C(F)(F)F)cn(C)c4=O)C(F)C3)cc12]

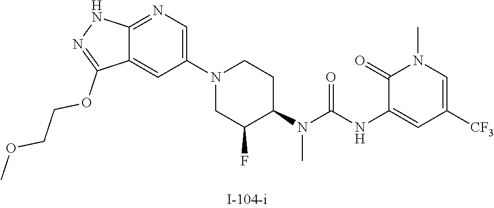 [SMILES: COCCOc1n[nH]c2ncc(N3CC[C@@H](N(C)C(=O)Nc4cc(C(F)(F)F)cn(C)c4=O)[C@@H](F)C3)cc12]

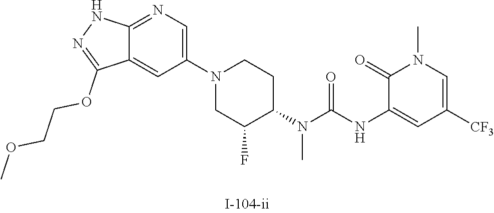 [SMILES: COCCOc1n[nH]c2ncc(N3CC[C@H](N(C)C(=O)Nc4cc(C(F)(F)F)cn(C)c4=O)[C@H](F)C3)cc12]

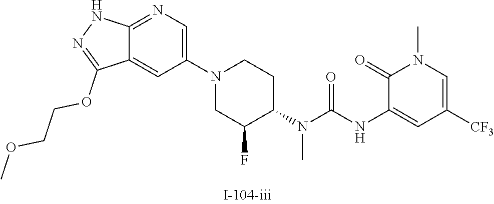 [SMILES: COCCOc1n[nH]c2ncc(N3CC[C@H](N(C)C(=O)Nc4cc(C(F)(F)F)cn(C)c4=O)[C@@H](F)C3)cc12]

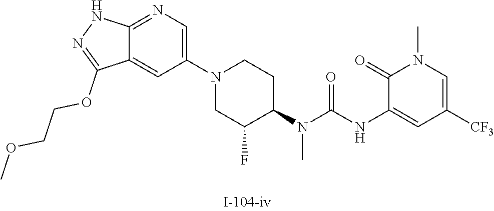 [SMILES: COCCOc1n[nH]c2ncc(N3CC[C@@H](N(C)C(=O)Nc4cc(C(F)(F)F)cn(C)c4=O)[C@H](F)C3)cc12]

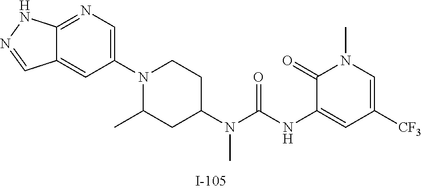 [SMILES: CC1CC(N(C)C(=O)Nc2cc(C(F)(F)F)cn(C)c2=O)CCN1c1cnc2[nH]ncc2c1]

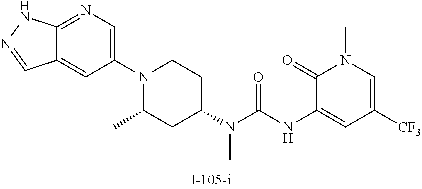 [SMILES: C[C@H]1C[C@@H](N(C)C(=O)Nc2cc(C(F)(F)F)cn(C)c2=O)CCN1c1cnc2[nH]ncc2c1]

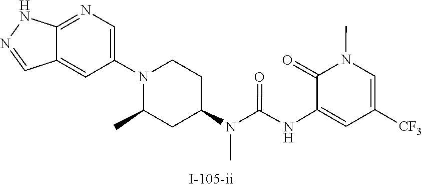 [SMILES: C[C@@H]1C[C@H](N(C)C(=O)Nc2cc(C(F)(F)F)cn(C)c2=O)CCN1c1cnc2[nH]ncc2c1]

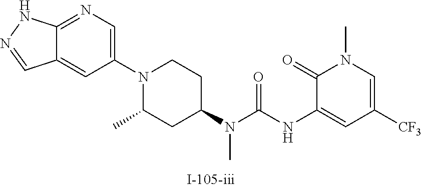 [SMILES: C[C@H]1C[C@H](N(C)C(=O)Nc2cc(C(F)(F)F)cn(C)c2=O)CCN1c1cnc2[nH]ncc2c1]

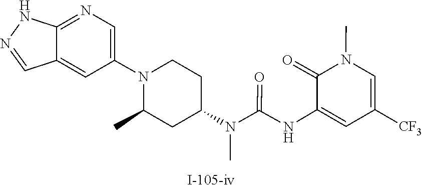 [SMILES: C[C@@H]1C[C@@H](N(C)C(=O)Nc2cc(C(F)(F)F)cn(C)c2=O)CCN1c1cnc2[nH]ncc2c1]

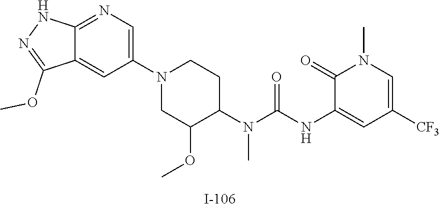 [SMILES: COc1n[nH]c2ncc(N3CCC(N(C)C(=O)Nc4cc(C(F)(F)F)cn(C)c4=O)C(OC)C3)cc12]

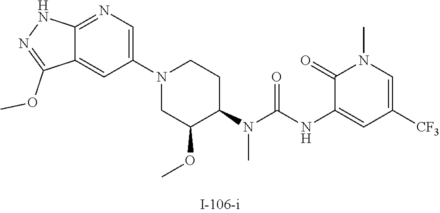 [SMILES: COc1n[nH]c2ncc(N3CC[C@@H](N(C)C(=O)Nc4cc(C(F)(F)F)cn(C)c4=O)[C@@H](OC)C3)cc12]

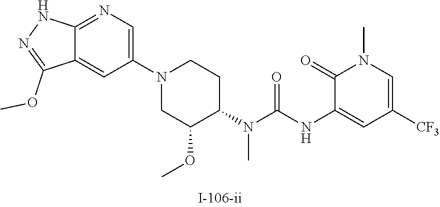 [SMILES: COc1n[nH]c2ncc(N3CC[C@H](N(C)C(=O)Nc4cc(C(F)(F)F)cn(C)c4=O)[C@H](OC)C3)cc12]

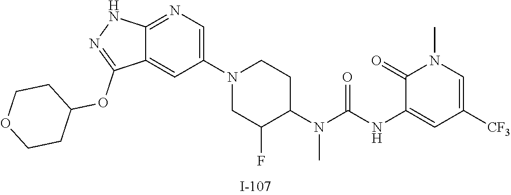 [SMILES: CN(C(=O)Nc1cc(C(F)(F)F)cn(C)c1=O)C1CCN(c2cnc3[nH]nc(OC4CCOCC4)c3c2)CC1F]

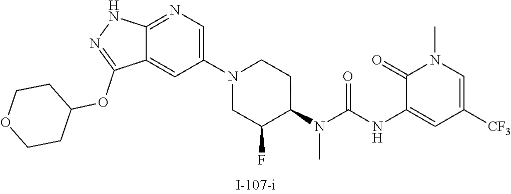 [SMILES: CN(C(=O)Nc1cc(C(F)(F)F)cn(C)c1=O)[C@@H]1CCN(c2cnc3[nH]nc(OC4CCOCC4)c3c2)C[C@@H]1F]

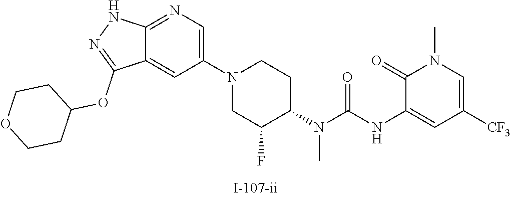 [SMILES: CN(C(=O)Nc1cc(C(F)(F)F)cn(C)c1=O)[C@H]1CCN(c2cnc3[nH]nc(OC4CCOCC4)c3c2)C[C@H]1F]

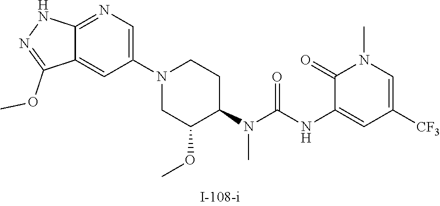 [SMILES: COc1n[nH]c2ncc(N3CC[C@@H](N(C)C(=O)Nc4cc(C(F)(F)F)cn(C)c4=O)[C@H](OC)C3)cc12]

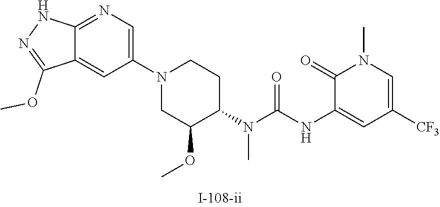 [SMILES: COc1n[nH]c2ncc(N3CC[C@H](N(C)C(=O)Nc4cc(C(F)(F)F)cn(C)c4=O)[C@@H](OC)C3)cc12]

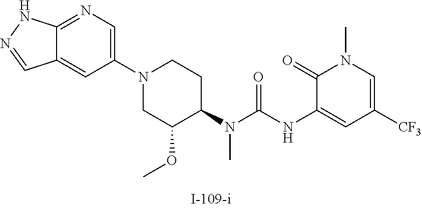 [SMILES: CO[C@@H]1CN(c2cnc3[nH]ncc3c2)CC[C@H]1N(C)C(=O)Nc1cc(C(F)(F)F)cn(C)c1=O]

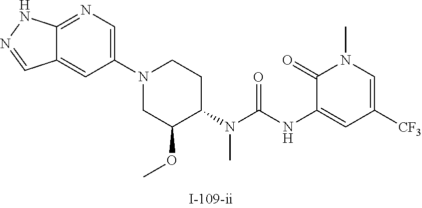 [SMILES: CO[C@H]1CN(c2cnc3[nH]ncc3c2)CC[C@@H]1N(C)C(=O)Nc1cc(C(F)(F)F)cn(C)c1=O]

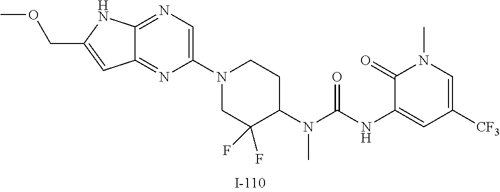 [SMILES: COCc1cc2nc(N3CCC(N(C)C(=O)Nc4cc(C(F)(F)F)cn(C)c4=O)C(F)(F)C3)cnc2[nH]1]

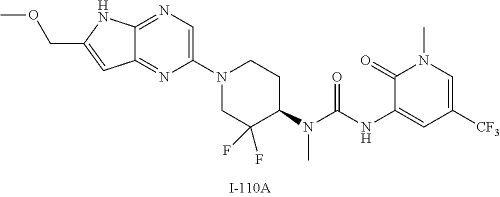 [SMILES: COCc1cc2nc(N3CC[C@@H](N(C)C(=O)Nc4cc(C(F)(F)F)cn(C)c4=O)C(F)(F)C3)cnc2[nH]1]

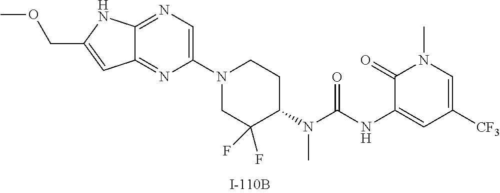 [SMILES: COCc1cc2nc(N3CC[C@H](N(C)C(=O)Nc4cc(C(F)(F)F)cn(C)c4=O)C(F)(F)C3)cnc2[nH]1]

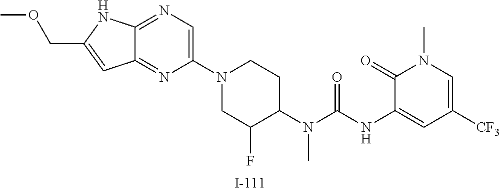 [SMILES: COCc1cc2nc(N3CCC(N(C)C(=O)Nc4cc(C(F)(F)F)cn(C)c4=O)C(F)C3)cnc2[nH]1]

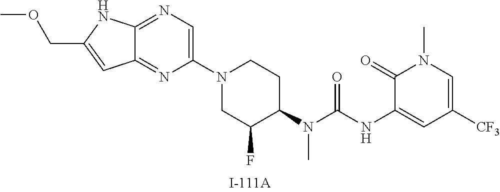 [SMILES: COCc1cc2nc(N3CC[C@@H](N(C)C(=O)Nc4cc(C(F)(F)F)cn(C)c4=O)[C@@H](F)C3)cnc2[nH]1]

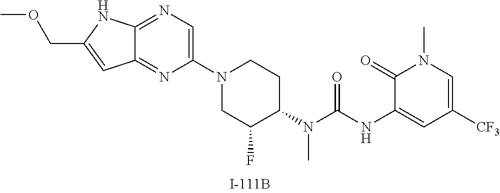 [SMILES: COCc1cc2nc(N3CC[C@H](N(C)C(=O)Nc4cc(C(F)(F)F)cn(C)c4=O)[C@H](F)C3)cnc2[nH]1]

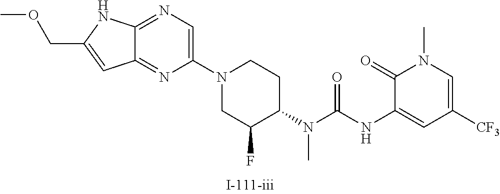 [SMILES: COCc1cc2nc(N3CC[C@H](N(C)C(=O)Nc4cc(C(F)(F)F)cn(C)c4=O)[C@@H](F)C3)cnc2[nH]1]

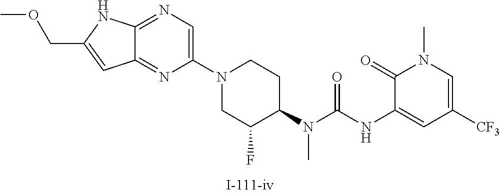 [SMILES: COCc1cc2nc(N3CC[C@@H](N(C)C(=O)Nc4cc(C(F)(F)F)cn(C)c4=O)[C@H](F)C3)cnc2[nH]1]

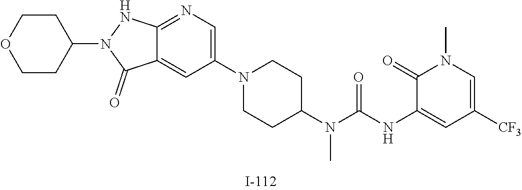 [SMILES: CN(C(=O)Nc1cc(C(F)(F)F)cn(C)c1=O)C1CCN(c2cnc3[nH]n(C4CCOCC4)c(=O)c3c2)CC1]

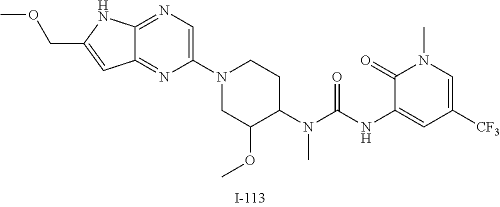 [SMILES: COCc1cc2nc(N3CCC(N(C)C(=O)Nc4cc(C(F)(F)F)cn(C)c4=O)C(OC)C3)cnc2[nH]1]

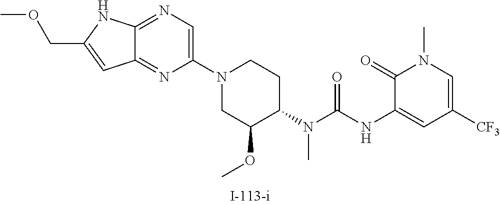 [SMILES: COCc1cc2nc(N3CC[C@H](N(C)C(=O)Nc4cc(C(F)(F)F)cn(C)c4=O)[C@@H](OC)C3)cnc2[nH]1]

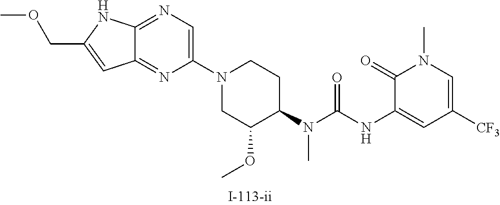 [SMILES: COCc1cc2nc(N3CC[C@@H](N(C)C(=O)Nc4cc(C(F)(F)F)cn(C)c4=O)[C@H](OC)C3)cnc2[nH]1]

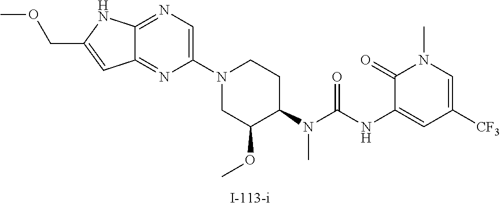 [SMILES: COCc1cc2nc(N3CC[C@@H](N(C)C(=O)Nc4cc(C(F)(F)F)cn(C)c4=O)[C@@H](OC)C3)cnc2[nH]1]

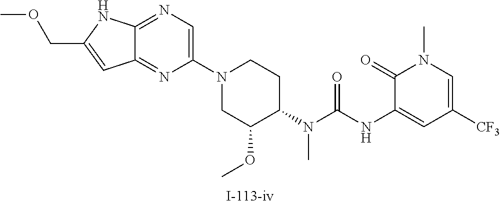 [SMILES: COCc1cc2nc(N3CC[C@H](N(C)C(=O)Nc4cc(C(F)(F)F)cn(C)c4=O)[C@H](OC)C3)cnc2[nH]1]

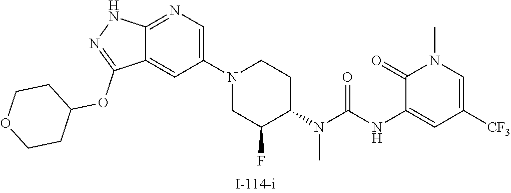 [SMILES: CN(C(=O)Nc1cc(C(F)(F)F)cn(C)c1=O)[C@H]1CCN(c2cnc3[nH]nc(OC4CCOCC4)c3c2)C[C@@H]1F]

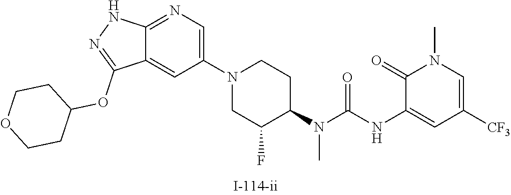 [SMILES: CN(C(=O)Nc1cc(C(F)(F)F)cn(C)c1=O)[C@@H]1CCN(c2cnc3[nH]nc(OC4CCOCC4)c3c2)C[C@H]1F]

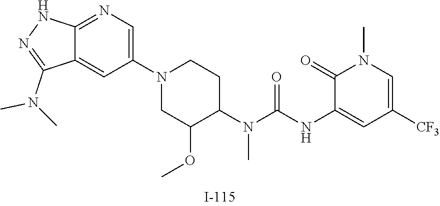 [SMILES: COC1CN(c2cnc3[nH]nc(N(C)C)c3c2)CCC1N(C)C(=O)Nc1cc(C(F)(F)F)cn(C)c1=O]

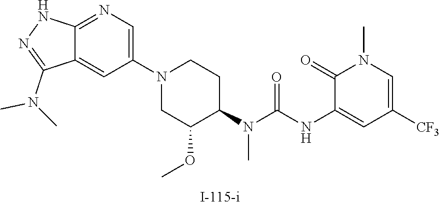 [SMILES: CO[C@@H]1CN(c2cnc3[nH]nc(N(C)C)c3c2)CC[C@H]1N(C)C(=O)Nc1cc(C(F)(F)F)cn(C)c1=O]

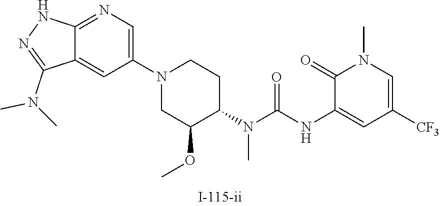 [SMILES: CO[C@H]1CN(c2cnc3[nH]nc(N(C)C)c3c2)CC[C@@H]1N(C)C(=O)Nc1cc(C(F)(F)F)cn(C)c1=O]

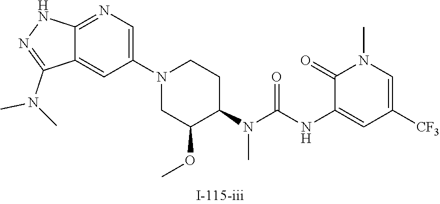 [SMILES: CO[C@H]1CN(c2cnc3[nH]nc(N(C)C)c3c2)CC[C@H]1N(C)C(=O)Nc1cc(C(F)(F)F)cn(C)c1=O]

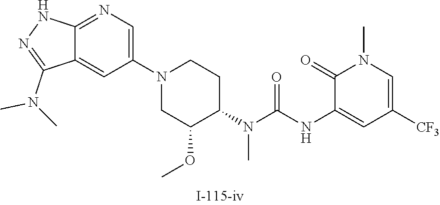 [SMILES: CO[C@@H]1CN(c2cnc3[nH]nc(N(C)C)c3c2)CC[C@@H]1N(C)C(=O)Nc1cc(C(F)(F)F)cn(C)c1=O]

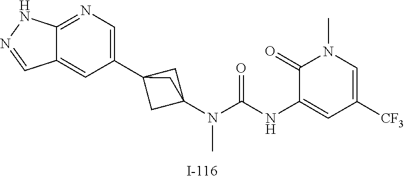 [SMILES: CN(C(=O)Nc1cc(C(F)(F)F)cn(C)c1=O)C12CC(c3cnc4[nH]ncc4c3)(C1)C2]

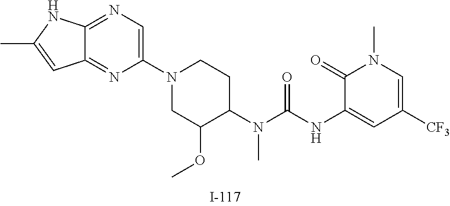 [SMILES: COC1CN(c2cnc3[nH]c(C)cc3n2)CCC1N(C)C(=O)Nc1cc(C(F)(F)F)cn(C)c1=O]

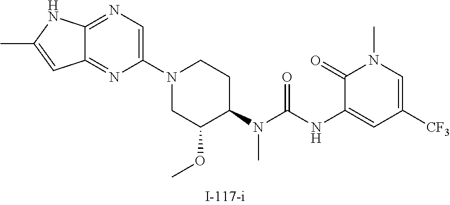 [SMILES: CO[C@@H]1CN(c2cnc3[nH]c(C)cc3n2)CC[C@H]1N(C)C(=O)Nc1cc(C(F)(F)F)cn(C)c1=O]

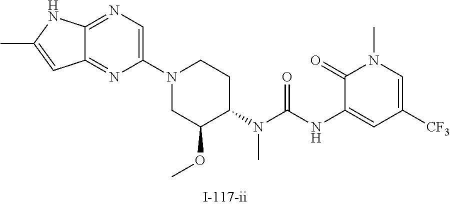 [SMILES: CO[C@H]1CN(c2cnc3[nH]c(C)cc3n2)CC[C@@H]1N(C)C(=O)Nc1cc(C(F)(F)F)cn(C)c1=O]

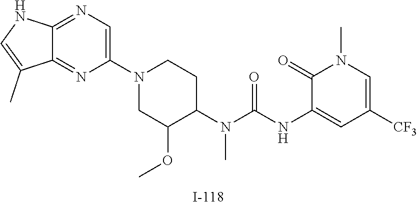 [SMILES: COC1CN(c2cnc3[nH]cc(C)c3n2)CCC1N(C)C(=O)Nc1cc(C(F)(F)F)cn(C)c1=O]

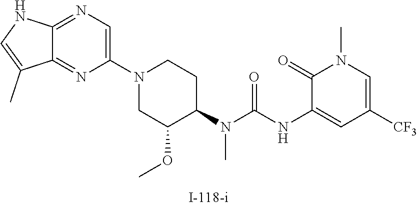 [SMILES: CO[C@@H]1CN(c2cnc3[nH]cc(C)c3n2)CC[C@H]1N(C)C(=O)Nc1cc(C(F)(F)F)cn(C)c1=O]

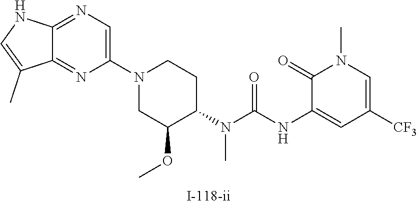 [SMILES: CO[C@H]1CN(c2cnc3[nH]cc(C)c3n2)CC[C@@H]1N(C)C(=O)Nc1cc(C(F)(F)F)cn(C)c1=O]

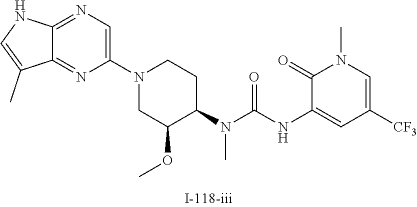 [SMILES: CO[C@H]1CN(c2cnc3[nH]cc(C)c3n2)CC[C@H]1N(C)C(=O)Nc1cc(C(F)(F)F)cn(C)c1=O]

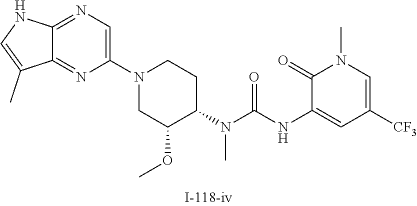 [SMILES: CO[C@@H]1CN(c2cnc3[nH]cc(C)c3n2)CC[C@@H]1N(C)C(=O)Nc1cc(C(F)(F)F)cn(C)c1=O]

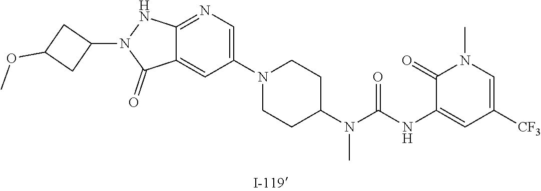 [SMILES: COC1CC(n2[nH]c3ncc(N4CCC(N(C)C(=O)Nc5cc(C(F)(F)F)cn(C)c5=O)CC4)cc3c2=O)C1]

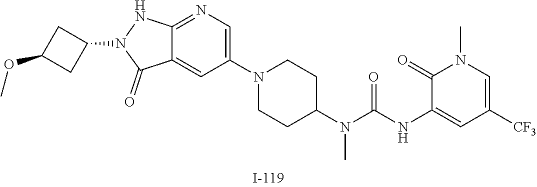 [SMILES: CO[C@H]1C[C@H](n2[nH]c3ncc(N4CCC(N(C)C(=O)Nc5cc(C(F)(F)F)cn(C)c5=O)CC4)cc3c2=O)C1]

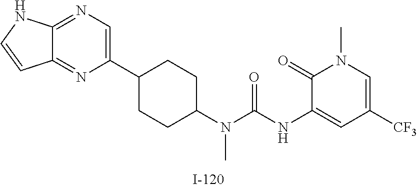 [SMILES: CN(C(=O)Nc1cc(C(F)(F)F)cn(C)c1=O)C1CCC(c2cnc3[nH]ccc3n2)CC1]

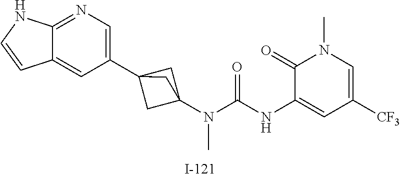 [SMILES: CN(C(=O)Nc1cc(C(F)(F)F)cn(C)c1=O)C12CC(c3cnc4[nH]ccc4c3)(C1)C2]

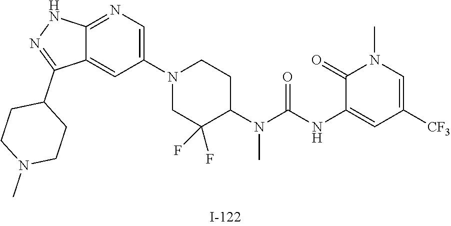 [SMILES: CN1CCC(c2n[nH]c3ncc(N4CCC(N(C)C(=O)Nc5cc(C(F)(F)F)cn(C)c5=O)C(F)(F)C4)cc23)CC1]

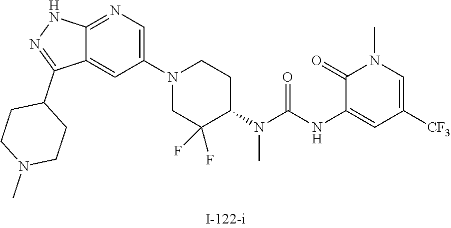 [SMILES: CN1CCC(c2n[nH]c3ncc(N4CC[C@H](N(C)C(=O)Nc5cc(C(F)(F)F)cn(C)c5=O)C(F)(F)C4)cc23)CC1]

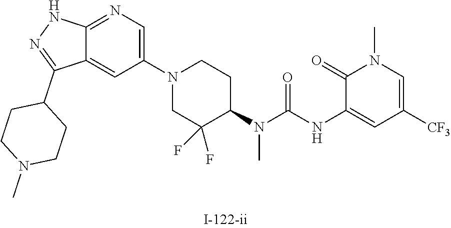 [SMILES: CN1CCC(c2n[nH]c3ncc(N4CC[C@@H](N(C)C(=O)Nc5cc(C(F)(F)F)cn(C)c5=O)C(F)(F)C4)cc23)CC1]

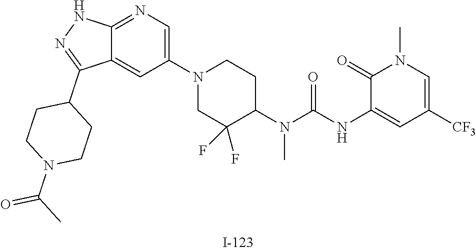 [SMILES: CC(=O)N1CCC(c2n[nH]c3ncc(N4CCC(N(C)C(=O)Nc5cc(C(F)(F)F)cn(C)c5=O)C(F)(F)C4)cc23)CC1]

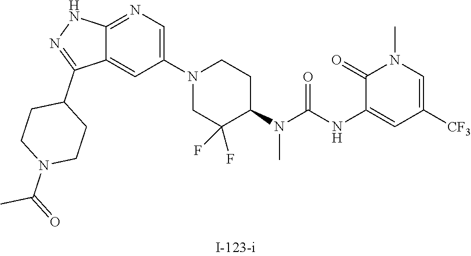 [SMILES: CC(=O)N1CCC(c2n[nH]c3ncc(N4CC[C@@H](N(C)C(=O)Nc5cc(C(F)(F)F)cn(C)c5=O)C(F)(F)C4)cc23)CC1]

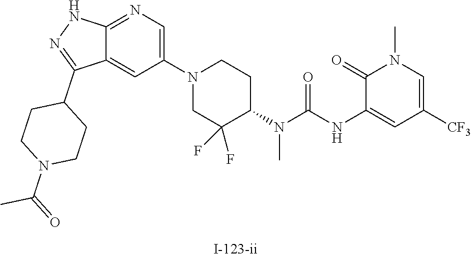 [SMILES: CC(=O)N1CCC(c2n[nH]c3ncc(N4CC[C@H](N(C)C(=O)Nc5cc(C(F)(F)F)cn(C)c5=O)C(F)(F)C4)cc23)CC1]

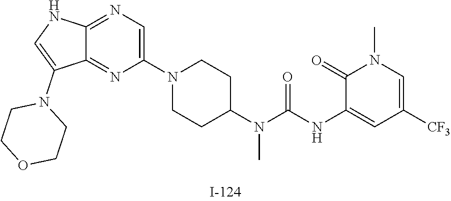 [SMILES: CN(C(=O)Nc1cc(C(F)(F)F)cn(C)c1=O)C1CCN(c2cnc3[nH]cc(N4CCOCC4)c3n2)CC1]

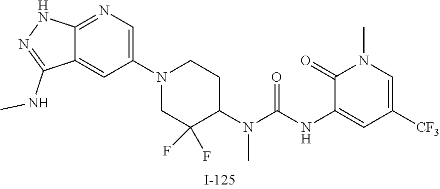 [SMILES: CNc1n[nH]c2ncc(N3CCC(N(C)C(=O)Nc4cc(C(F)(F)F)cn(C)c4=O)C(F)(F)C3)cc12]

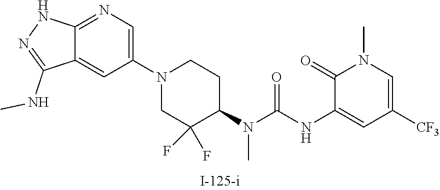 [SMILES: CNc1n[nH]c2ncc(N3CC[C@@H](N(C)C(=O)Nc4cc(C(F)(F)F)cn(C)c4=O)C(F)(F)C3)cc12]

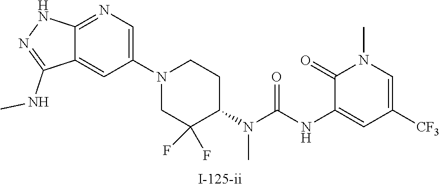 [SMILES: CNc1n[nH]c2ncc(N3CC[C@H](N(C)C(=O)Nc4cc(C(F)(F)F)cn(C)c4=O)C(F)(F)C3)cc12]

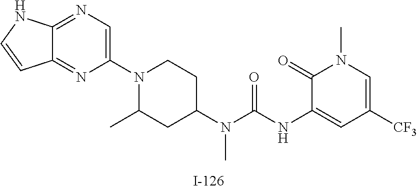 [SMILES: CC1CC(N(C)C(=O)Nc2cc(C(F)(F)F)cn(C)c2=O)CCN1c1cnc2[nH]ccc2n1]

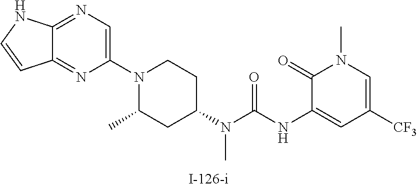 [SMILES: C[C@H]1C[C@@H](N(C)C(=O)Nc2cc(C(F)(F)F)cn(C)c2=O)CCN1c1cnc2[nH]ccc2n1]

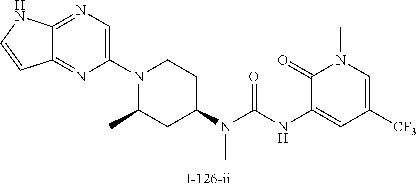 [SMILES: C[C@@H]1C[C@H](N(C)C(=O)Nc2cc(C(F)(F)F)cn(C)c2=O)CCN1c1cnc2[nH]ccc2n1]

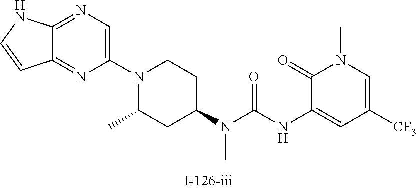 [SMILES: C[C@H]1C[C@H](N(C)C(=O)Nc2cc(C(F)(F)F)cn(C)c2=O)CCN1c1cnc2[nH]ccc2n1]

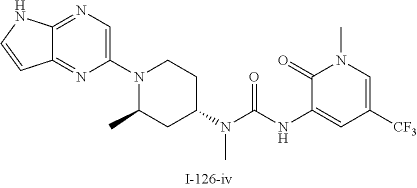 [SMILES: C[C@@H]1C[C@@H](N(C)C(=O)Nc2cc(C(F)(F)F)cn(C)c2=O)CCN1c1cnc2[nH]ccc2n1]

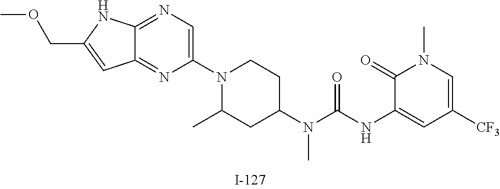 [SMILES: COCc1cc2nc(N3CCC(N(C)C(=O)Nc4cc(C(F)(F)F)cn(C)c4=O)CC3C)cnc2[nH]1]

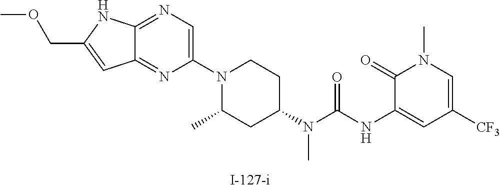 [SMILES: COCc1cc2nc(N3CC[C@H](N(C)C(=O)Nc4cc(C(F)(F)F)cn(C)c4=O)C[C@@H]3C)cnc2[nH]1]

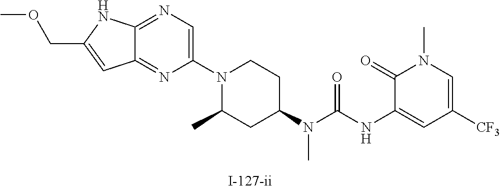 [SMILES: COCc1cc2nc(N3CC[C@@H](N(C)C(=O)Nc4cc(C(F)(F)F)cn(C)c4=O)C[C@H]3C)cnc2[nH]1]

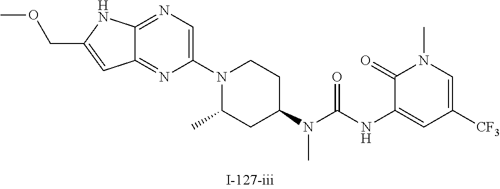 [SMILES: COCc1cc2nc(N3CC[C@@H](N(C)C(=O)Nc4cc(C(F)(F)F)cn(C)c4=O)C[C@@H]3C)cnc2[nH]1]

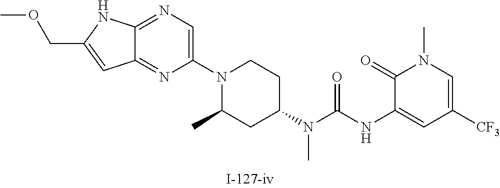 [SMILES: COCc1cc2nc(N3CC[C@H](N(C)C(=O)Nc4cc(C(F)(F)F)cn(C)c4=O)C[C@H]3C)cnc2[nH]1]

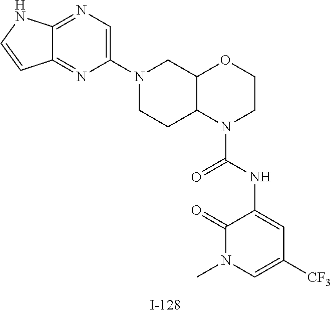 [SMILES: Cn1cc(C(F)(F)F)cc(NC(=O)N2CCOC3CN(c4cnc5[nH]ccc5n4)CCC32)c1=O]

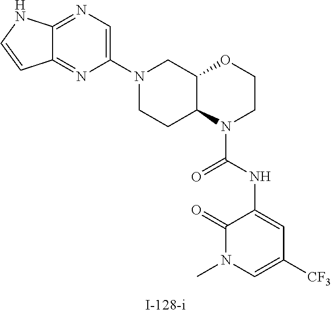 [SMILES: Cn1cc(C(F)(F)F)cc(NC(=O)N2CCO[C@H]3CN(c4cnc5[nH]ccc5n4)CC[C@@H]32)c1=O]

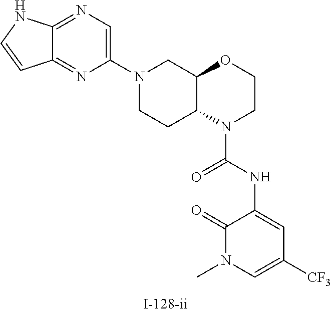 [SMILES: Cn1cc(C(F)(F)F)cc(NC(=O)N2CCO[C@@H]3CN(c4cnc5[nH]ccc5n4)CC[C@H]32)c1=O]

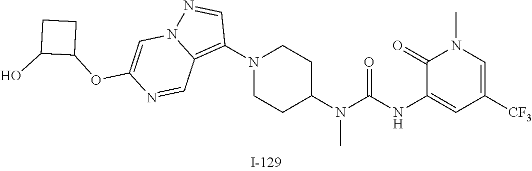 [SMILES: CN(C(=O)Nc1cc(C(F)(F)F)cn(C)c1=O)C1CCN(c2cnn3cc(OC4CCC4O)ncc23)CC1]

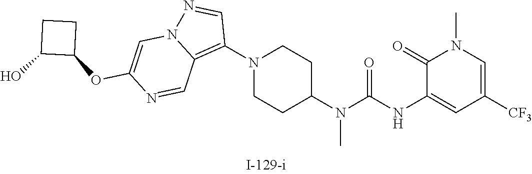 [SMILES: CN(C(=O)Nc1cc(C(F)(F)F)cn(C)c1=O)C1CCN(c2cnn3cc(O[C@@H]4CC[C@H]4O)ncc23)CC1]

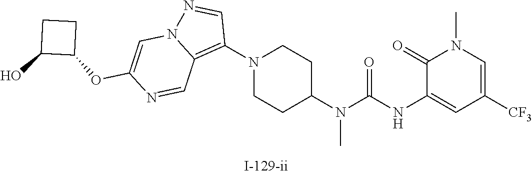 [SMILES: CN(C(=O)Nc1cc(C(F)(F)F)cn(C)c1=O)C1CCN(c2cnn3cc(O[C@H]4CC[C@@H]4O)ncc23)CC1]

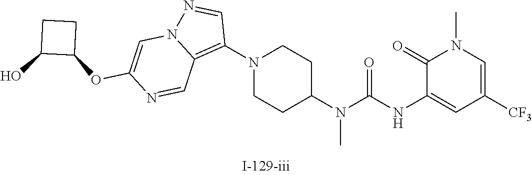 [SMILES: CN(C(=O)Nc1cc(C(F)(F)F)cn(C)c1=O)C1CCN(c2cnn3cc(O[C@@H]4CC[C@@H]4O)ncc23)CC1]

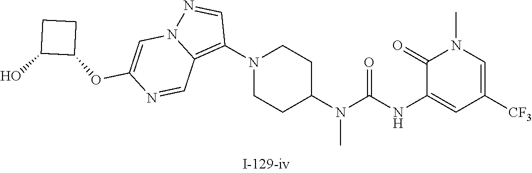 [SMILES: CN(C(=O)Nc1cc(C(F)(F)F)cn(C)c1=O)C1CCN(c2cnn3cc(O[C@H]4CC[C@H]4O)ncc23)CC1]

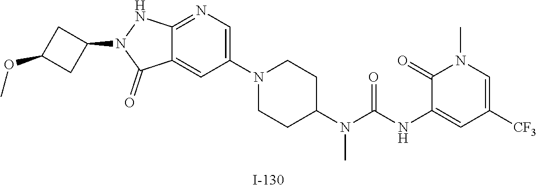 [SMILES: CO[C@H]1C[C@@H](n2[nH]c3ncc(N4CCC(N(C)C(=O)Nc5cc(C(F)(F)F)cn(C)c5=O)CC4)cc3c2=O)C1]

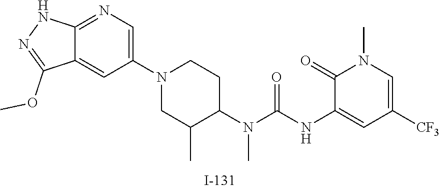 [SMILES: COc1n[nH]c2ncc(N3CCC(N(C)C(=O)Nc4cc(C(F)(F)F)cn(C)c4=O)C(C)C3)cc12]

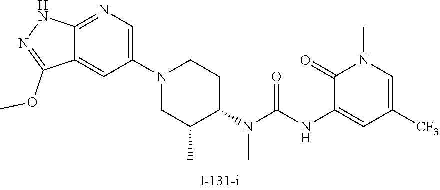 [SMILES: COc1n[nH]c2ncc(N3CC[C@H](N(C)C(=O)Nc4cc(C(F)(F)F)cn(C)c4=O)[C@H](C)C3)cc12]

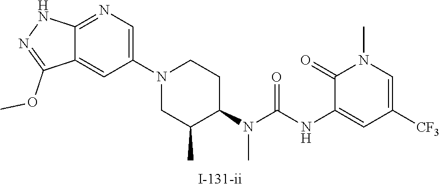 [SMILES: COc1n[nH]c2ncc(N3CC[C@@H](N(C)C(=O)Nc4cc(C(F)(F)F)cn(C)c4=O)[C@@H](C)C3)cc12]

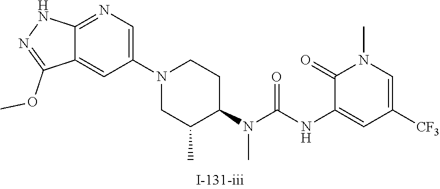 [SMILES: COc1n[nH]c2ncc(N3CC[C@@H](N(C)C(=O)Nc4cc(C(F)(F)F)cn(C)c4=O)[C@H](C)C3)cc12]

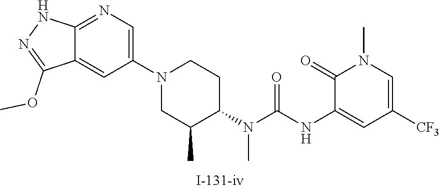 [SMILES: COc1n[nH]c2ncc(N3CC[C@H](N(C)C(=O)Nc4cc(C(F)(F)F)cn(C)c4=O)[C@@H](C)C3)cc12]

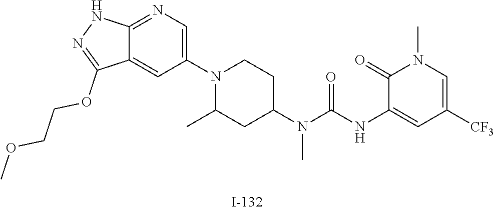 [SMILES: COCCOc1n[nH]c2ncc(N3CCC(N(C)C(=O)Nc4cc(C(F)(F)F)cn(C)c4=O)CC3C)cc12]

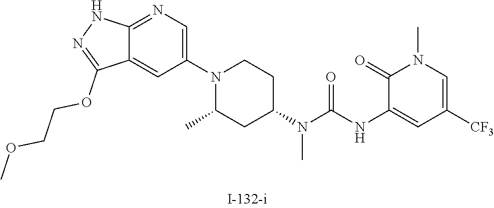 [SMILES: COCCOc1n[nH]c2ncc(N3CC[C@H](N(C)C(=O)Nc4cc(C(F)(F)F)cn(C)c4=O)C[C@@H]3C)cc12]

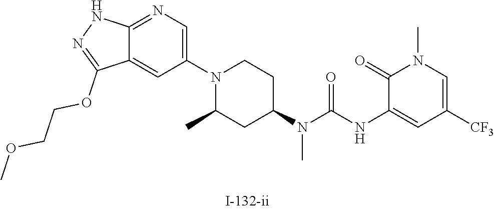 [SMILES: COCCOc1n[nH]c2ncc(N3CC[C@@H](N(C)C(=O)Nc4cc(C(F)(F)F)cn(C)c4=O)C[C@H]3C)cc12]

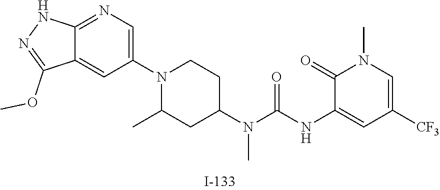 [SMILES: COc1n[nH]c2ncc(N3CCC(N(C)C(=O)Nc4cc(C(F)(F)F)cn(C)c4=O)CC3C)cc12]

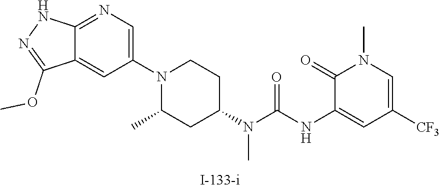 [SMILES: COc1n[nH]c2ncc(N3CC[C@H](N(C)C(=O)Nc4cc(C(F)(F)F)cn(C)c4=O)C[C@@H]3C)cc12]

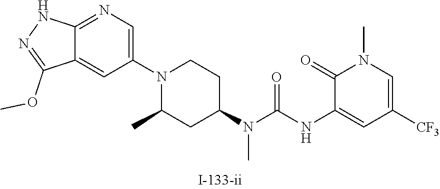 [SMILES: COc1n[nH]c2ncc(N3CC[C@@H](N(C)C(=O)Nc4cc(C(F)(F)F)cn(C)c4=O)C[C@H]3C)cc12]

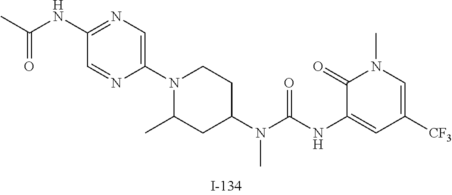 [SMILES: CC(=O)Nc1cnc(N2CCC(N(C)C(=O)Nc3cc(C(F)(F)F)cn(C)c3=O)CC2C)cn1]

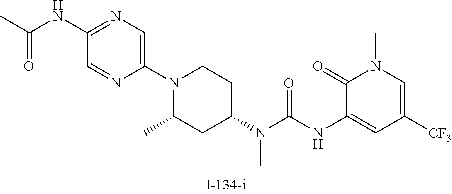 [SMILES: CC(=O)Nc1cnc(N2CC[C@H](N(C)C(=O)Nc3cc(C(F)(F)F)cn(C)c3=O)C[C@@H]2C)cn1]

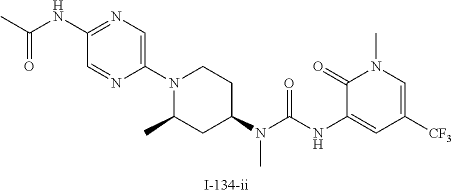 [SMILES: CC(=O)Nc1cnc(N2CC[C@@H](N(C)C(=O)Nc3cc(C(F)(F)F)cn(C)c3=O)C[C@H]2C)cn1]

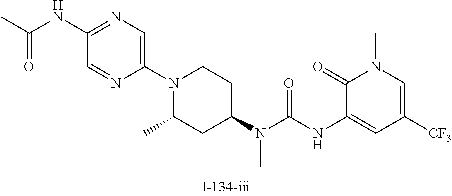 [SMILES: CC(=O)Nc1cnc(N2CC[C@@H](N(C)C(=O)Nc3cc(C(F)(F)F)cn(C)c3=O)C[C@@H]2C)cn1]

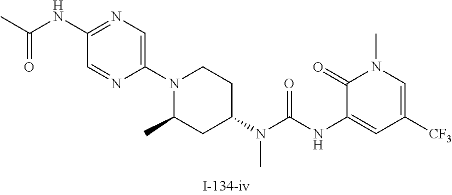 [SMILES: CC(=O)Nc1cnc(N2CC[C@H](N(C)C(=O)Nc3cc(C(F)(F)F)cn(C)c3=O)C[C@H]2C)cn1]

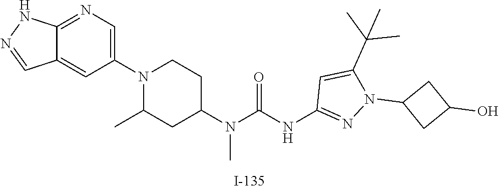 [SMILES: CC1CC(N(C)C(=O)Nc2cc(C(C)(C)C)n(C3CC(O)C3)n2)CCN1c1cnc2[nH]ncc2c1]

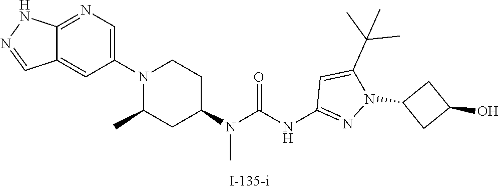 [SMILES: C[C@@H]1C[C@H](N(C)C(=O)Nc2cc(C(C)(C)C)n([C@H]3C[C@H](O)C3)n2)CCN1c1cnc2[nH]ncc2c1]

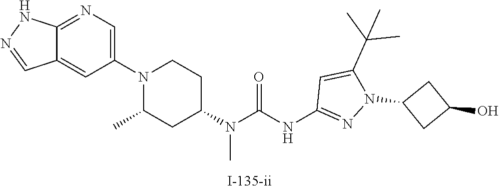 [SMILES: C[C@H]1C[C@@H](N(C)C(=O)Nc2cc(C(C)(C)C)n([C@H]3C[C@H](O)C3)n2)CCN1c1cnc2[nH]ncc2c1]

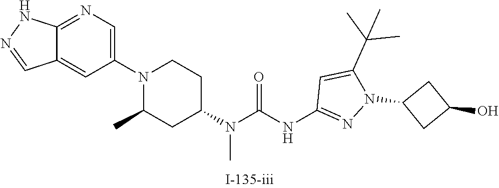 [SMILES: C[C@@H]1C[C@@H](N(C)C(=O)Nc2cc(C(C)(C)C)n([C@H]3C[C@H](O)C3)n2)CCN1c1cnc2[nH]ncc2c1]

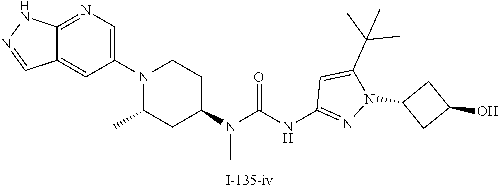 [SMILES: C[C@H]1C[C@H](N(C)C(=O)Nc2cc(C(C)(C)C)n([C@H]3C[C@H](O)C3)n2)CCN1c1cnc2[nH]ncc2c1]

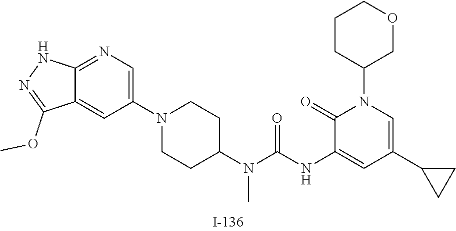 [SMILES: COc1n[nH]c2ncc(N3CCC(N(C)C(=O)Nc4cc(C5CC5)cn(C5CCCOC5)c4=O)CC3)cc12]

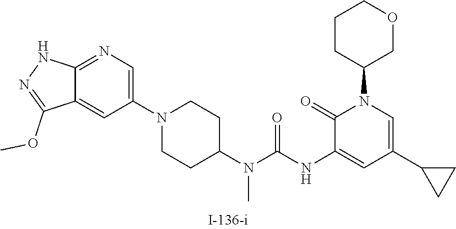 [SMILES: COc1n[nH]c2ncc(N3CCC(N(C)C(=O)Nc4cc(C5CC5)cn([C@H]5CCCOC5)c4=O)CC3)cc12]

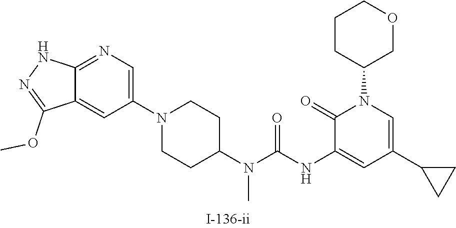 [SMILES: COc1n[nH]c2ncc(N3CCC(N(C)C(=O)Nc4cc(C5CC5)cn([C@@H]5CCCOC5)c4=O)CC3)cc12]

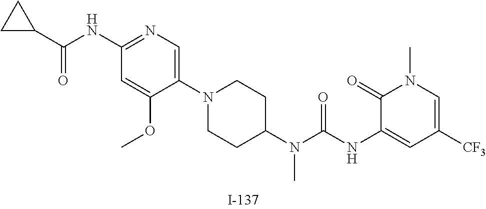 [SMILES: COc1cc(NC(=O)C2CC2)ncc1N1CCC(N(C)C(=O)Nc2cc(C(F)(F)F)cn(C)c2=O)CC1]

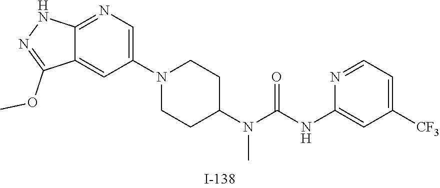 [SMILES: COc1n[nH]c2ncc(N3CCC(N(C)C(=O)Nc4cc(C(F)(F)F)ccn4)CC3)cc12]

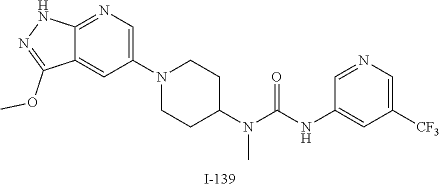 [SMILES: COc1n[nH]c2ncc(N3CCC(N(C)C(=O)Nc4cncc(C(F)(F)F)c4)CC3)cc12]

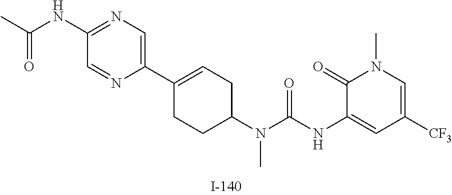 [SMILES: CC(=O)Nc1cnc(C2=CCC(N(C)C(=O)Nc3cc(C(F)(F)F)cn(C)c3=O)CC2)cn1]

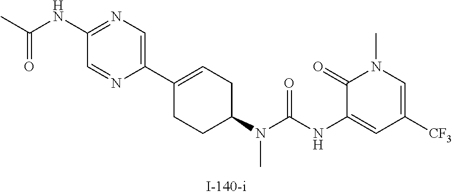 [SMILES: CC(=O)Nc1cnc(C2=CC[C@@H](N(C)C(=O)Nc3cc(C(F)(F)F)cn(C)c3=O)CC2)cn1]

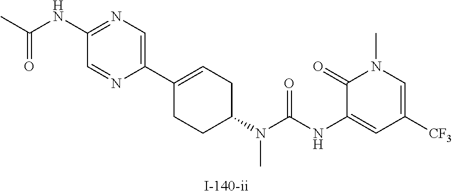 [SMILES: CC(=O)Nc1cnc(C2=CC[C@H](N(C)C(=O)Nc3cc(C(F)(F)F)cn(C)c3=O)CC2)cn1]

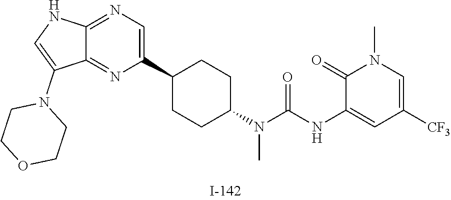 [SMILES: Cn1cc(C(F)(F)F)cc(NC(=O)N(C)[C@H]2CC[C@H](c3cnc4[nH]cc(N5CCOCC5)c4n3)CC2)c1=O]

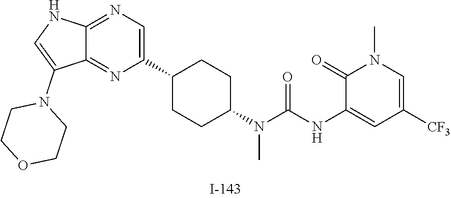 [SMILES: Cn1cc(C(F)(F)F)cc(NC(=O)N(C)[C@H]2CC[C@@H](c3cnc4[nH]cc(N5CCOCC5)c4n3)CC2)c1=O]

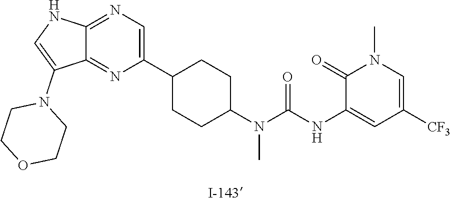 [SMILES: CN(C(=O)Nc1cc(C(F)(F)F)cn(C)c1=O)C1CCC(c2cnc3[nH]cc(N4CCOCC4)c3n2)CC1]

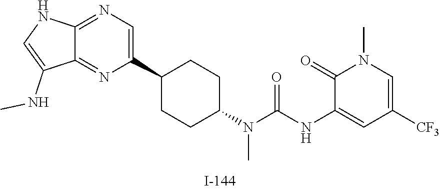 [SMILES: CNc1c[nH]c2ncc([C@H]3CC[C@H](N(C)C(=O)Nc4cc(C(F)(F)F)cn(C)c4=O)CC3)nc12]

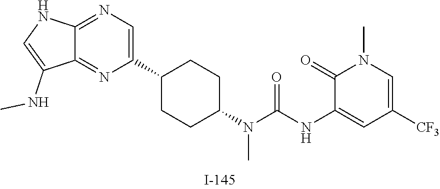 [SMILES: CNc1c[nH]c2ncc([C@H]3CC[C@@H](N(C)C(=O)Nc4cc(C(F)(F)F)cn(C)c4=O)CC3)nc12]

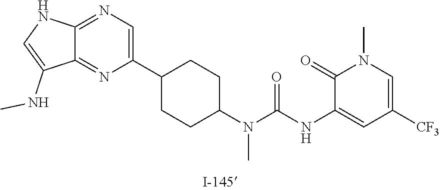 [SMILES: CNc1c[nH]c2ncc(C3CCC(N(C)C(=O)Nc4cc(C(F)(F)F)cn(C)c4=O)CC3)nc12]

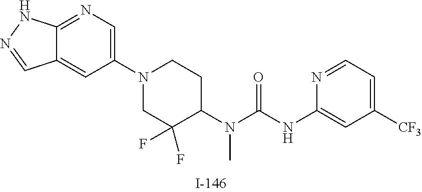 [SMILES: CN(C(=O)Nc1cc(C(F)(F)F)ccn1)C1CCN(c2cnc3[nH]ncc3c2)CC1(F)F]

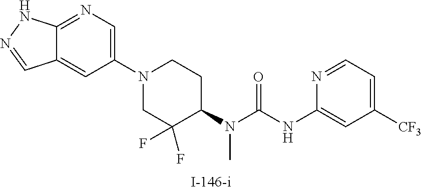 [SMILES: CN(C(=O)Nc1cc(C(F)(F)F)ccn1)[C@@H]1CCN(c2cnc3[nH]ncc3c2)CC1(F)F]

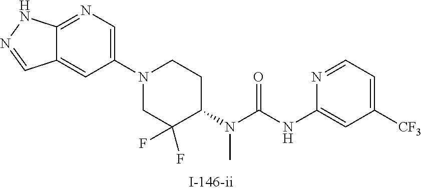 [SMILES: CN(C(=O)Nc1cc(C(F)(F)F)ccn1)[C@H]1CCN(c2cnc3[nH]ncc3c2)CC1(F)F]

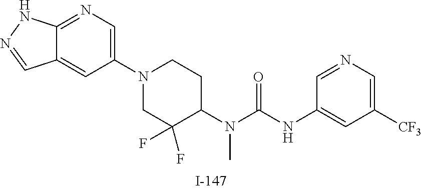 [SMILES: CN(C(=O)Nc1cncc(C(F)(F)F)c1)C1CCN(c2cnc3[nH]ncc3c2)CC1(F)F]

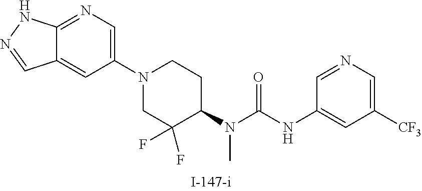 [SMILES: CN(C(=O)Nc1cncc(C(F)(F)F)c1)[C@@H]1CCN(c2cnc3[nH]ncc3c2)CC1(F)F]

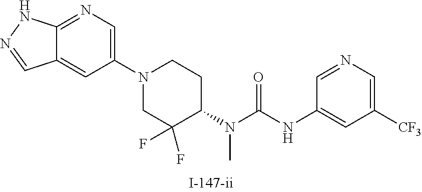 [SMILES: CN(C(=O)Nc1cncc(C(F)(F)F)c1)[C@H]1CCN(c2cnc3[nH]ncc3c2)CC1(F)F]

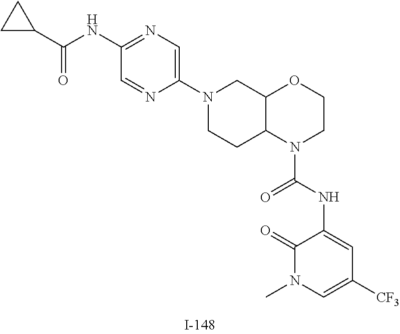 [SMILES: Cn1cc(C(F)(F)F)cc(NC(=O)N2CCOC3CN(c4cnc(NC(=O)C5CC5)cn4)CCC32)c1=O]

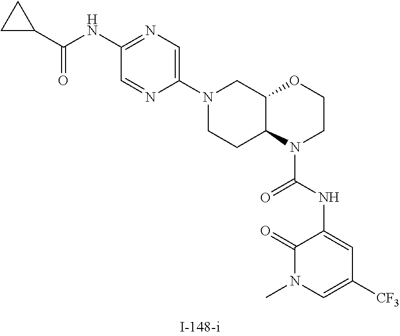 [SMILES: Cn1cc(C(F)(F)F)cc(NC(=O)N2CCO[C@H]3CN(c4cnc(NC(=O)C5CC5)cn4)CC[C@@H]32)c1=O]

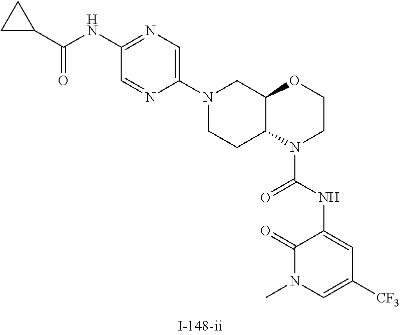 [SMILES: Cn1cc(C(F)(F)F)cc(NC(=O)N2CCO[C@@H]3CN(c4cnc(NC(=O)C5CC5)cn4)CC[C@H]32)c1=O]

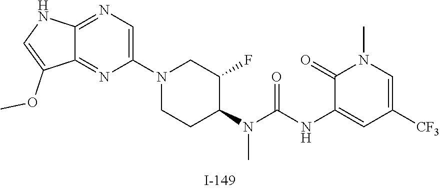 [SMILES: COc1c[nH]c2ncc(N3CC[C@H](N(C)C(=O)Nc4cc(C(F)(F)F)cn(C)c4=O)[C@@H](F)C3)nc12]

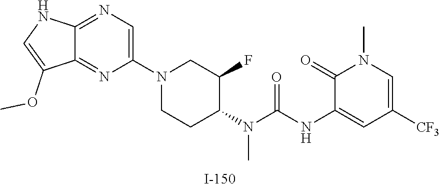 [SMILES: COc1c[nH]c2ncc(N3CC[C@@H](N(C)C(=O)Nc4cc(C(F)(F)F)cn(C)c4=O)[C@H](F)C3)nc12]

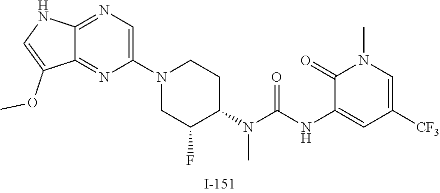 [SMILES: COc1c[nH]c2ncc(N3CC[C@H](N(C)C(=O)Nc4cc(C(F)(F)F)cn(C)c4=O)[C@H](F)C3)nc12]

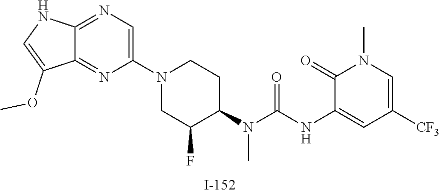 [SMILES: COc1c[nH]c2ncc(N3CC[C@@H](N(C)C(=O)Nc4cc(C(F)(F)F)cn(C)c4=O)[C@@H](F)C3)nc12]

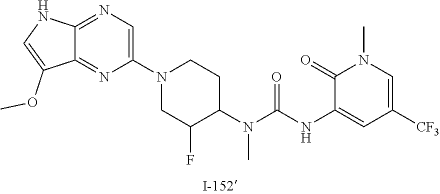 [SMILES: COc1c[nH]c2ncc(N3CCC(N(C)C(=O)Nc4cc(C(F)(F)F)cn(C)c4=O)C(F)C3)nc12]

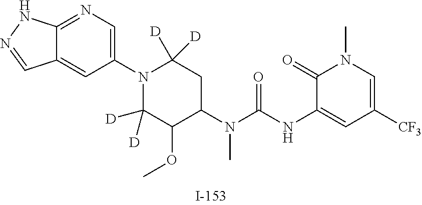 [SMILES: [2H]C1([2H])CC(N(C)C(=O)Nc2cc(C(F)(F)F)cn(C)c2=O)C(OC)C([2H])([2H])N1c1cnc2[nH]ncc2c1]

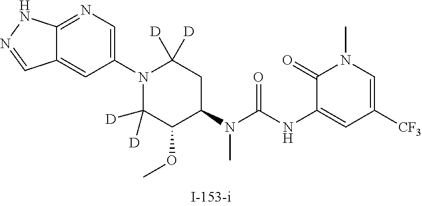 [SMILES: [2H]C1([2H])C[C@@H](N(C)C(=O)Nc2cc(C(F)(F)F)cn(C)c2=O)[C@H](OC)C([2H])([2H])N1c1cnc2[nH]ncc2c1]

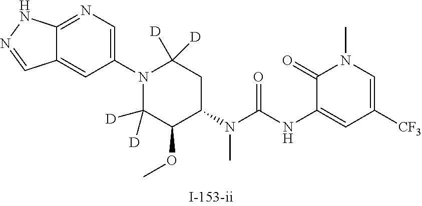 [SMILES: [2H]C1([2H])C[C@H](N(C)C(=O)Nc2cc(C(F)(F)F)cn(C)c2=O)[C@@H](OC)C([2H])([2H])N1c1cnc2[nH]ncc2c1]

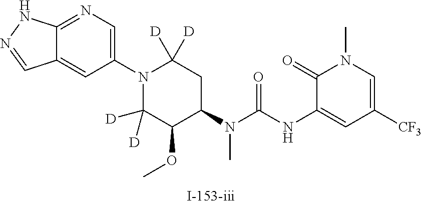 [SMILES: [2H]C1([2H])C[C@@H](N(C)C(=O)Nc2cc(C(F)(F)F)cn(C)c2=O)[C@@H](OC)C([2H])([2H])N1c1cnc2[nH]ncc2c1]

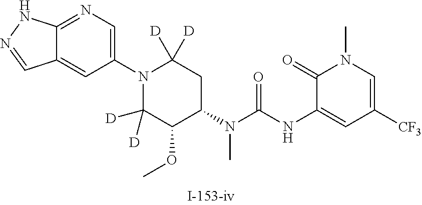 [SMILES: [2H]C1([2H])C[C@H](N(C)C(=O)Nc2cc(C(F)(F)F)cn(C)c2=O)[C@H](OC)C([2H])([2H])N1c1cnc2[nH]ncc2c1]

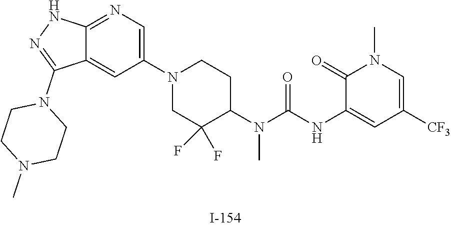 [SMILES: CN1CCN(c2n[nH]c3ncc(N4CCC(N(C)C(=O)Nc5cc(C(F)(F)F)cn(C)c5=O)C(F)(F)C4)cc23)CC1]

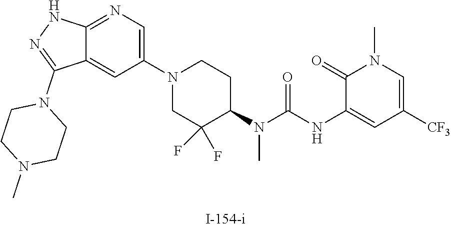 [SMILES: CN1CCN(c2n[nH]c3ncc(N4CC[C@@H](N(C)C(=O)Nc5cc(C(F)(F)F)cn(C)c5=O)C(F)(F)C4)cc23)CC1]

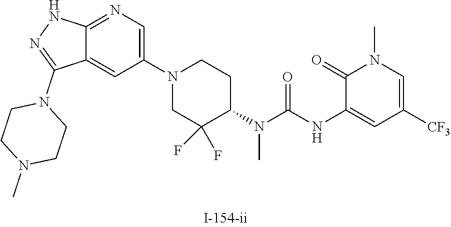 [SMILES: CN1CCN(c2n[nH]c3ncc(N4CC[C@H](N(C)C(=O)Nc5cc(C(F)(F)F)cn(C)c5=O)C(F)(F)C4)cc23)CC1]

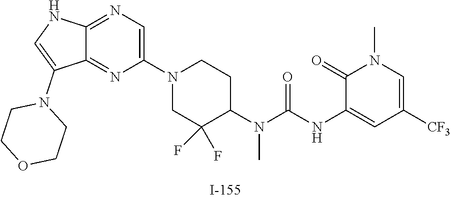 [SMILES: CN(C(=O)Nc1cc(C(F)(F)F)cn(C)c1=O)C1CCN(c2cnc3[nH]cc(N4CCOCC4)c3n2)CC1(F)F]

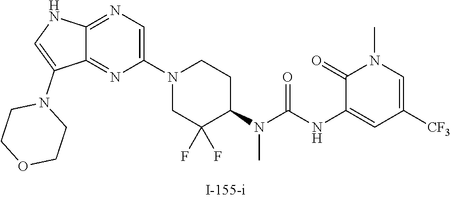 [SMILES: CN(C(=O)Nc1cc(C(F)(F)F)cn(C)c1=O)[C@@H]1CCN(c2cnc3[nH]cc(N4CCOCC4)c3n2)CC1(F)F]

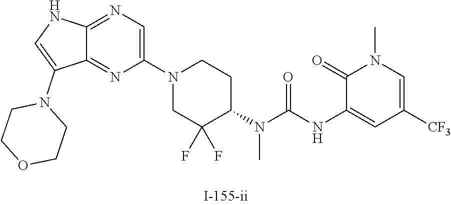 [SMILES: CN(C(=O)Nc1cc(C(F)(F)F)cn(C)c1=O)[C@H]1CCN(c2cnc3[nH]cc(N4CCOCC4)c3n2)CC1(F)F]

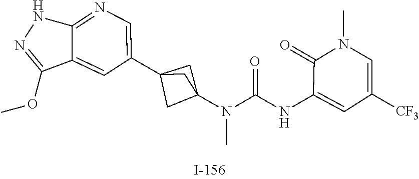 [SMILES: COc1n[nH]c2ncc(C34CC(N(C)C(=O)Nc5cc(C(F)(F)F)cn(C)c5=O)(C3)C4)cc12]

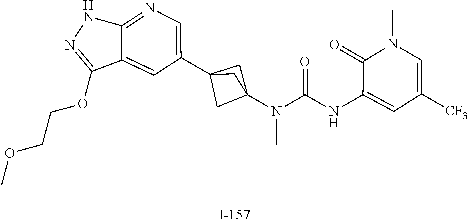 [SMILES: COCCOc1n[nH]c2ncc(C34CC(N(C)C(=O)Nc5cc(C(F)(F)F)cn(C)c5=O)(C3)C4)cc12]

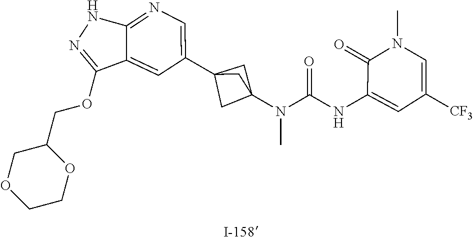 [SMILES: CN(C(=O)Nc1cc(C(F)(F)F)cn(C)c1=O)C12CC(c3cnc4[nH]nc(OCC5COCCO5)c4c3)(C1)C2]

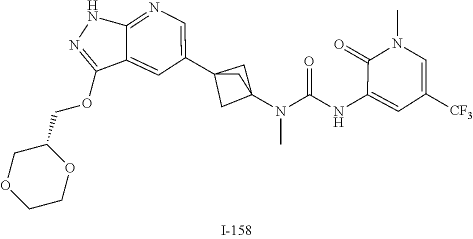 [SMILES: CN(C(=O)Nc1cc(C(F)(F)F)cn(C)c1=O)C12CC(c3cnc4[nH]nc(OC[C@H]5COCCO5)c4c3)(C1)C2]

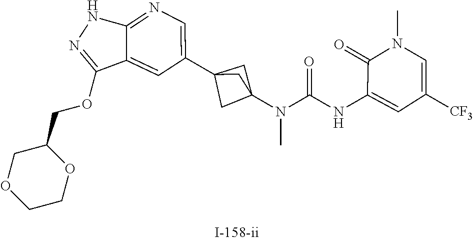 [SMILES: CN(C(=O)Nc1cc(C(F)(F)F)cn(C)c1=O)C12CC(c3cnc4[nH]nc(OC[C@@H]5COCCO5)c4c3)(C1)C2]

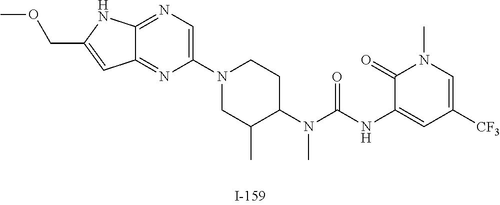 [SMILES: COCc1cc2nc(N3CCC(N(C)C(=O)Nc4cc(C(F)(F)F)cn(C)c4=O)C(C)C3)cnc2[nH]1]

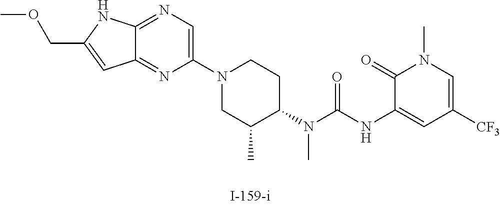 [SMILES: COCc1cc2nc(N3CC[C@H](N(C)C(=O)Nc4cc(C(F)(F)F)cn(C)c4=O)[C@H](C)C3)cnc2[nH]1]

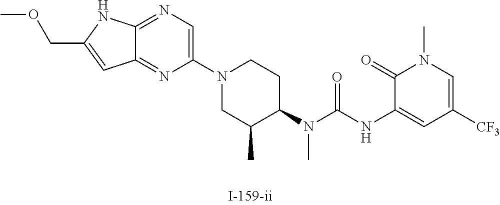 [SMILES: COCc1cc2nc(N3CC[C@@H](N(C)C(=O)Nc4cc(C(F)(F)F)cn(C)c4=O)[C@@H](C)C3)cnc2[nH]1]

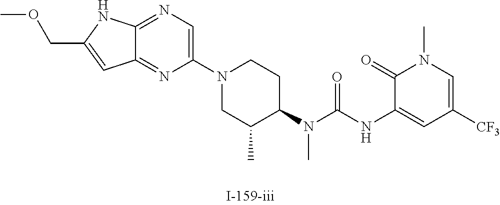 [SMILES: COCc1cc2nc(N3CC[C@@H](N(C)C(=O)Nc4cc(C(F)(F)F)cn(C)c4=O)[C@H](C)C3)cnc2[nH]1]

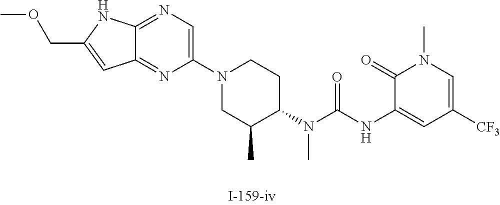 [SMILES: COCc1cc2nc(N3CC[C@H](N(C)C(=O)Nc4cc(C(F)(F)F)cn(C)c4=O)[C@@H](C)C3)cnc2[nH]1]

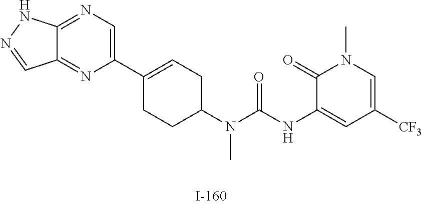 [SMILES: CN(C(=O)Nc1cc(C(F)(F)F)cn(C)c1=O)C1CC=C(c2cnc3[nH]ncc3n2)CC1]

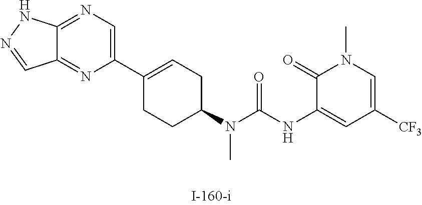 [SMILES: CN(C(=O)Nc1cc(C(F)(F)F)cn(C)c1=O)[C@@H]1CC=C(c2cnc3[nH]ncc3n2)CC1]

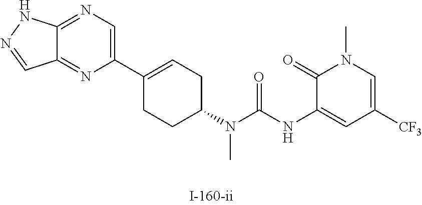 [SMILES: CN(C(=O)Nc1cc(C(F)(F)F)cn(C)c1=O)[C@H]1CC=C(c2cnc3[nH]ncc3n2)CC1]

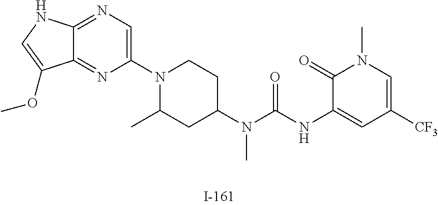 [SMILES: COc1c[nH]c2ncc(N3CCC(N(C)C(=O)Nc4cc(C(F)(F)F)cn(C)c4=O)CC3C)nc12]

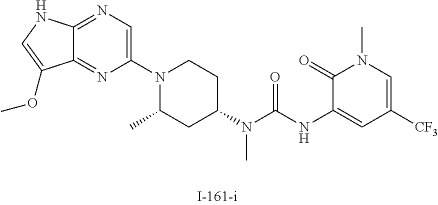 [SMILES: COc1c[nH]c2ncc(N3CC[C@H](N(C)C(=O)Nc4cc(C(F)(F)F)cn(C)c4=O)C[C@@H]3C)nc12]

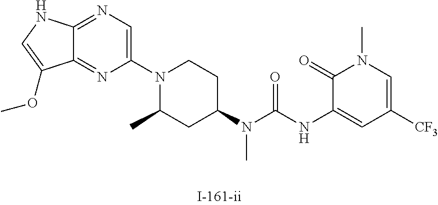 [SMILES: COc1c[nH]c2ncc(N3CC[C@@H](N(C)C(=O)Nc4cc(C(F)(F)F)cn(C)c4=O)C[C@H]3C)nc12]

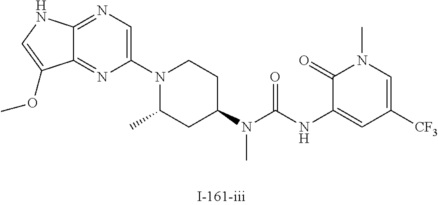 [SMILES: COc1c[nH]c2ncc(N3CC[C@@H](N(C)C(=O)Nc4cc(C(F)(F)F)cn(C)c4=O)C[C@@H]3C)nc12]

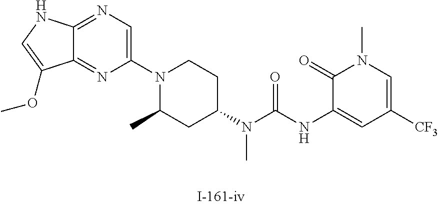 [SMILES: COc1c[nH]c2ncc(N3CC[C@H](N(C)C(=O)Nc4cc(C(F)(F)F)cn(C)c4=O)C[C@H]3C)nc12]

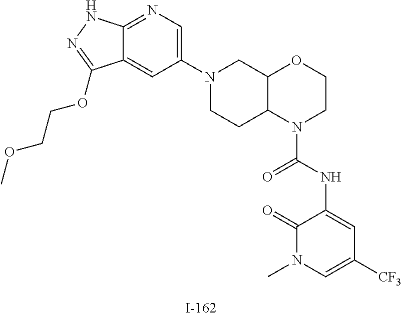 [SMILES: COCCOc1n[nH]c2ncc(N3CCC4C(C3)OCCN4C(=O)Nc3cc(C(F)(F)F)cn(C)c3=O)cc12]

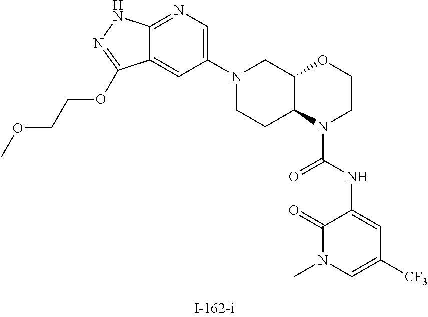 [SMILES: COCCOc1n[nH]c2ncc(N3CC[C@H]4[C@H](C3)OCCN4C(=O)Nc3cc(C(F)(F)F)cn(C)c3=O)cc12]

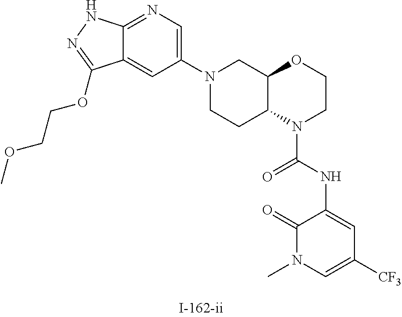 [SMILES: COCCOc1n[nH]c2ncc(N3CC[C@@H]4[C@@H](C3)OCCN4C(=O)Nc3cc(C(F)(F)F)cn(C)c3=O)cc12]

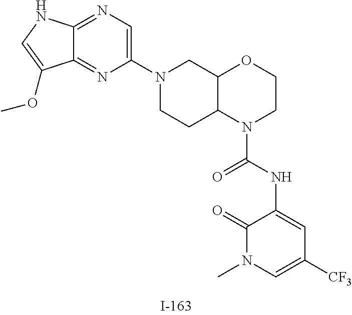 [SMILES: COc1c[nH]c2ncc(N3CCC4C(C3)OCCN4C(=O)Nc3cc(C(F)(F)F)cn(C)c3=O)nc12]

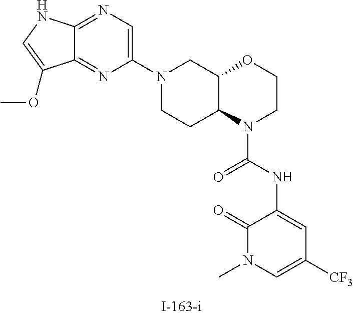 [SMILES: COc1c[nH]c2ncc(N3CC[C@H]4[C@H](C3)OCCN4C(=O)Nc3cc(C(F)(F)F)cn(C)c3=O)nc12]

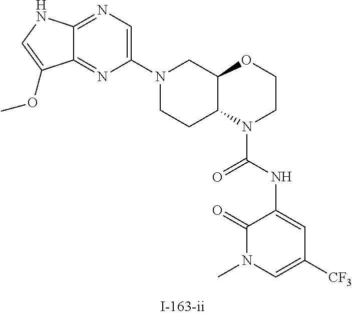 [SMILES: COc1c[nH]c2ncc(N3CC[C@@H]4[C@@H](C3)OCCN4C(=O)Nc3cc(C(F)(F)F)cn(C)c3=O)nc12]

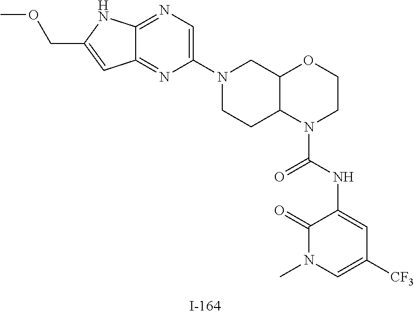 [SMILES: COCc1cc2nc(N3CCC4C(C3)OCCN4C(=O)Nc3cc(C(F)(F)F)cn(C)c3=O)cnc2[nH]1]

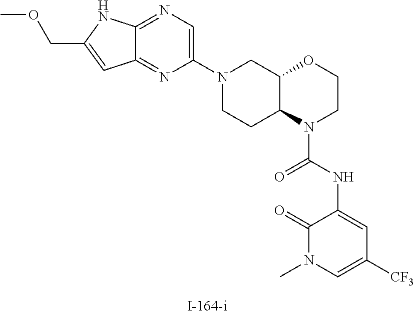 [SMILES: COCc1cc2nc(N3CC[C@H]4[C@H](C3)OCCN4C(=O)Nc3cc(C(F)(F)F)cn(C)c3=O)cnc2[nH]1]

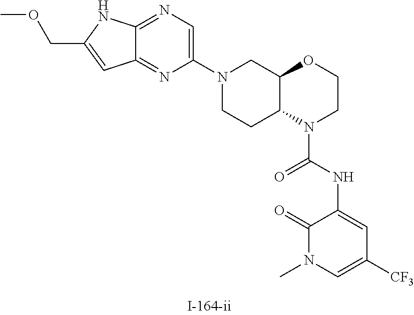 [SMILES: COCc1cc2nc(N3CC[C@@H]4[C@@H](C3)OCCN4C(=O)Nc3cc(C(F)(F)F)cn(C)c3=O)cnc2[nH]1]

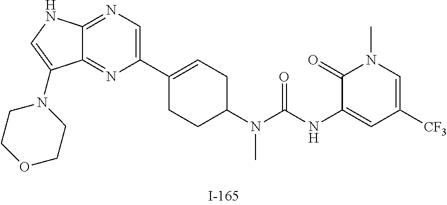 [SMILES: CN(C(=O)Nc1cc(C(F)(F)F)cn(C)c1=O)C1CC=C(c2cnc3[nH]cc(N4CCOCC4)c3n2)CC1]

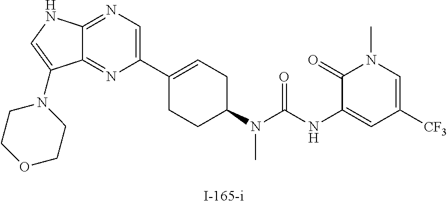 [SMILES: CN(C(=O)Nc1cc(C(F)(F)F)cn(C)c1=O)[C@@H]1CC=C(c2cnc3[nH]cc(N4CCOCC4)c3n2)CC1]

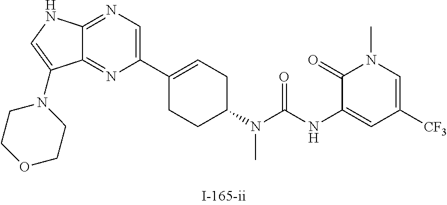 [SMILES: CN(C(=O)Nc1cc(C(F)(F)F)cn(C)c1=O)[C@H]1CC=C(c2cnc3[nH]cc(N4CCOCC4)c3n2)CC1]

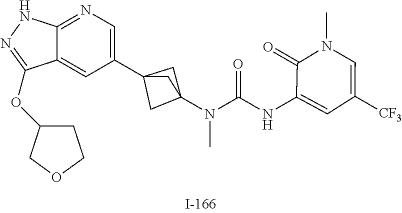 [SMILES: CN(C(=O)Nc1cc(C(F)(F)F)cn(C)c1=O)C12CC(c3cnc4[nH]nc(OC5CCOC5)c4c3)(C1)C2]

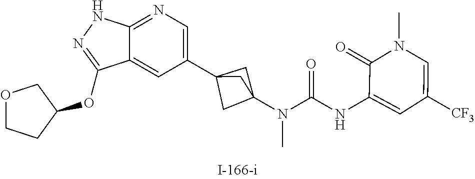 [SMILES: CN(C(=O)Nc1cc(C(F)(F)F)cn(C)c1=O)C12CC(c3cnc4[nH]nc(O[C@H]5CCOC5)c4c3)(C1)C2]

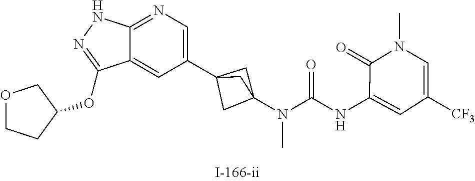 [SMILES: CN(C(=O)Nc1cc(C(F)(F)F)cn(C)c1=O)C12CC(c3cnc4[nH]nc(O[C@@H]5CCOC5)c4c3)(C1)C2]

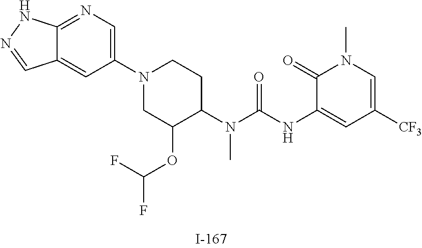 [SMILES: CN(C(=O)Nc1cc(C(F)(F)F)cn(C)c1=O)C1CCN(c2cnc3[nH]ncc3c2)CC1OC(F)F]

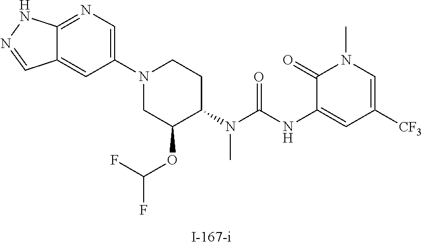 [SMILES: CN(C(=O)Nc1cc(C(F)(F)F)cn(C)c1=O)[C@H]1CCN(c2cnc3[nH]ncc3c2)C[C@@H]1OC(F)F]

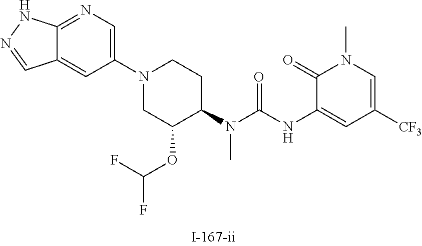 [SMILES: CN(C(=O)Nc1cc(C(F)(F)F)cn(C)c1=O)[C@@H]1CCN(c2cnc3[nH]ncc3c2)C[C@H]1OC(F)F]

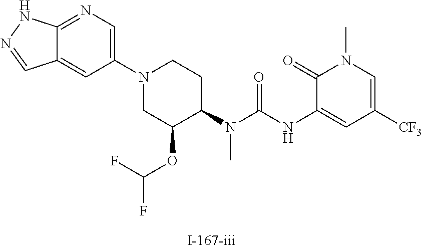 [SMILES: CN(C(=O)Nc1cc(C(F)(F)F)cn(C)c1=O)[C@@H]1CCN(c2cnc3[nH]ncc3c2)C[C@@H]1OC(F)F]

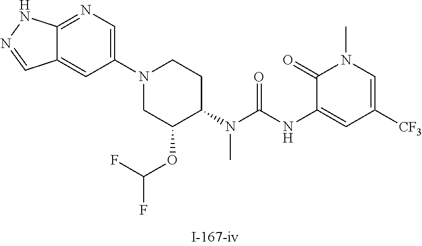 [SMILES: CN(C(=O)Nc1cc(C(F)(F)F)cn(C)c1=O)[C@H]1CCN(c2cnc3[nH]ncc3c2)C[C@H]1OC(F)F]

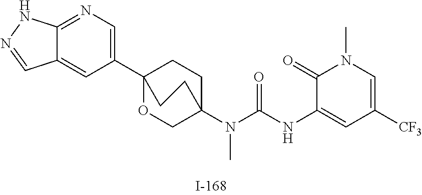 [SMILES: CN(C(=O)Nc1cc(C(F)(F)F)cn(C)c1=O)C12CCC(c3cnc4[nH]ncc4c3)(CC1)OC2]

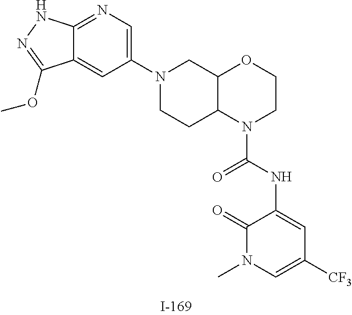 [SMILES: COc1n[nH]c2ncc(N3CCC4C(C3)OCCN4C(=O)Nc3cc(C(F)(F)F)cn(C)c3=O)cc12]

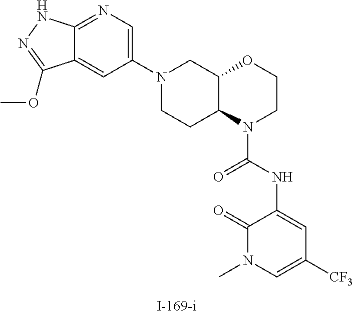 [SMILES: COc1n[nH]c2ncc(N3CC[C@H]4[C@H](C3)OCCN4C(=O)Nc3cc(C(F)(F)F)cn(C)c3=O)cc12]

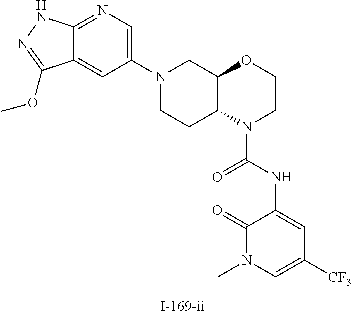 [SMILES: COc1n[nH]c2ncc(N3CC[C@@H]4[C@@H](C3)OCCN4C(=O)Nc3cc(C(F)(F)F)cn(C)c3=O)cc12]

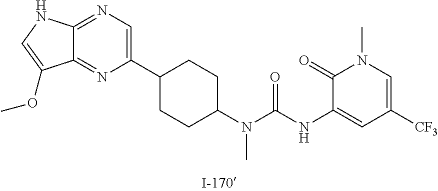 [SMILES: COc1c[nH]c2ncc(C3CCC(N(C)C(=O)Nc4cc(C(F)(F)F)cn(C)c4=O)CC3)nc12]

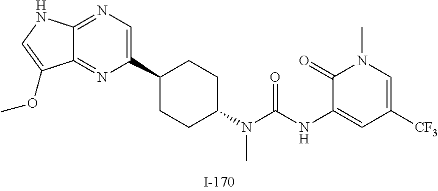 [SMILES: COc1c[nH]c2ncc([C@H]3CC[C@H](N(C)C(=O)Nc4cc(C(F)(F)F)cn(C)c4=O)CC3)nc12]

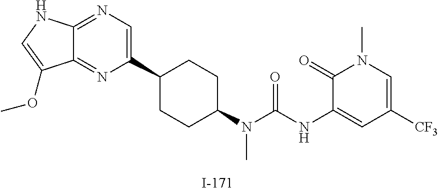 [SMILES: COc1c[nH]c2ncc([C@H]3CC[C@@H](N(C)C(=O)Nc4cc(C(F)(F)F)cn(C)c4=O)CC3)nc12]

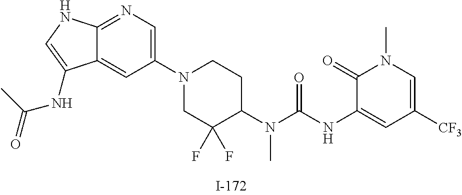 [SMILES: CC(=O)Nc1c[nH]c2ncc(N3CCC(N(C)C(=O)Nc4cc(C(F)(F)F)cn(C)c4=O)C(F)(F)C3)cc12]

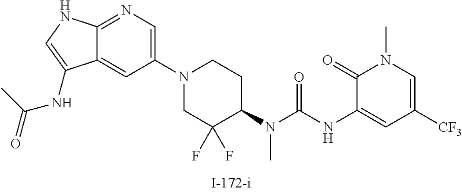 [SMILES: CC(=O)Nc1c[nH]c2ncc(N3CC[C@@H](N(C)C(=O)Nc4cc(C(F)(F)F)cn(C)c4=O)C(F)(F)C3)cc12]

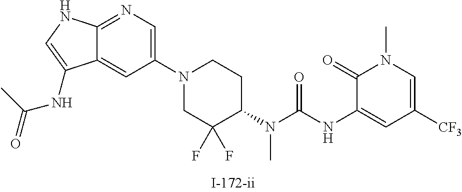 [SMILES: CC(=O)Nc1c[nH]c2ncc(N3CC[C@H](N(C)C(=O)Nc4cc(C(F)(F)F)cn(C)c4=O)C(F)(F)C3)cc12]

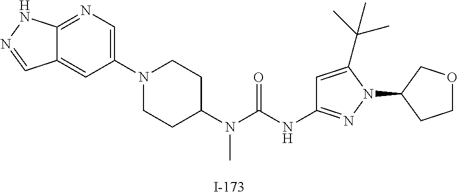 [SMILES: CN(C(=O)Nc1cc(C(C)(C)C)n([C@@H]2CCOC2)n1)C1CCN(c2cnc3[nH]ncc3c2)CC1]

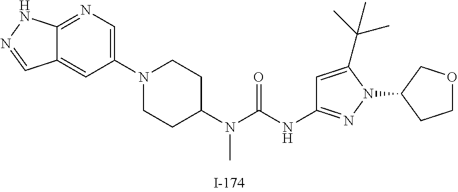 [SMILES: CN(C(=O)Nc1cc(C(C)(C)C)n([C@H]2CCOC2)n1)C1CCN(c2cnc3[nH]ncc3c2)CC1]

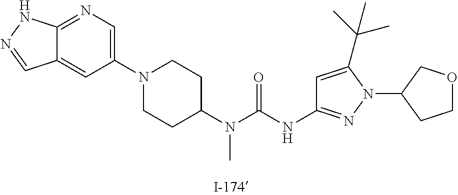 [SMILES: CN(C(=O)Nc1cc(C(C)(C)C)n(C2CCOC2)n1)C1CCN(c2cnc3[nH]ncc3c2)CC1]

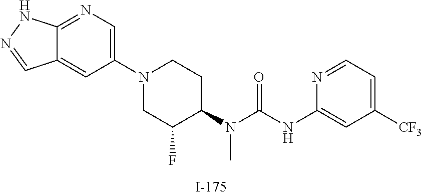 [SMILES: CN(C(=O)Nc1cc(C(F)(F)F)ccn1)[C@@H]1CCN(c2cnc3[nH]ncc3c2)C[C@H]1F]

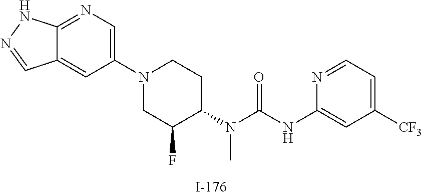 [SMILES: CN(C(=O)Nc1cc(C(F)(F)F)ccn1)[C@H]1CCN(c2cnc3[nH]ncc3c2)C[C@@H]1F]

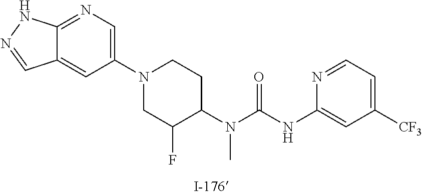 [SMILES: CN(C(=O)Nc1cc(C(F)(F)F)ccn1)C1CCN(c2cnc3[nH]ncc3c2)CC1F]

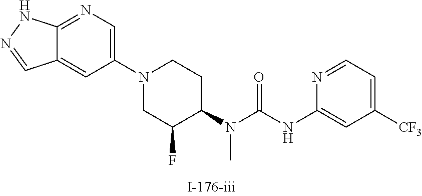 [SMILES: CN(C(=O)Nc1cc(C(F)(F)F)ccn1)[C@@H]1CCN(c2cnc3[nH]ncc3c2)C[C@@H]1F]

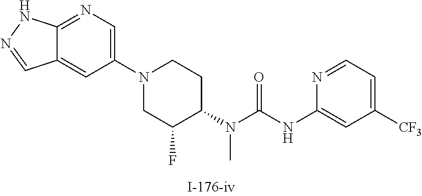 [SMILES: CN(C(=O)Nc1cc(C(F)(F)F)ccn1)[C@H]1CCN(c2cnc3[nH]ncc3c2)C[C@H]1F]

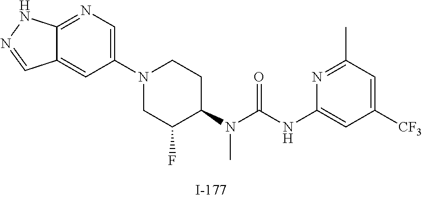 [SMILES: Cc1cc(C(F)(F)F)cc(NC(=O)N(C)[C@@H]2CCN(c3cnc4[nH]ncc4c3)C[C@H]2F)n1]

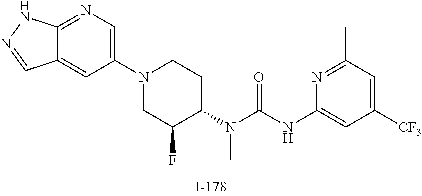 [SMILES: Cc1cc(C(F)(F)F)cc(NC(=O)N(C)[C@H]2CCN(c3cnc4[nH]ncc4c3)C[C@@H]2F)n1]

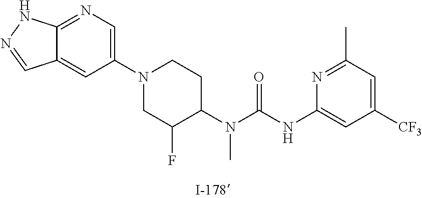 [SMILES: Cc1cc(C(F)(F)F)cc(NC(=O)N(C)C2CCN(c3cnc4[nH]ncc4c3)CC2F)n1]

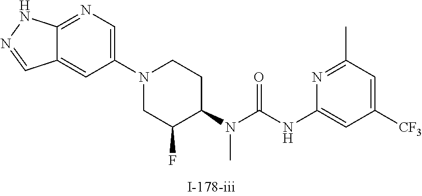 [SMILES: Cc1cc(C(F)(F)F)cc(NC(=O)N(C)[C@@H]2CCN(c3cnc4[nH]ncc4c3)C[C@@H]2F)n1]

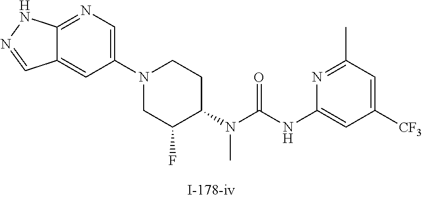 [SMILES: Cc1cc(C(F)(F)F)cc(NC(=O)N(C)[C@H]2CCN(c3cnc4[nH]ncc4c3)C[C@H]2F)n1]

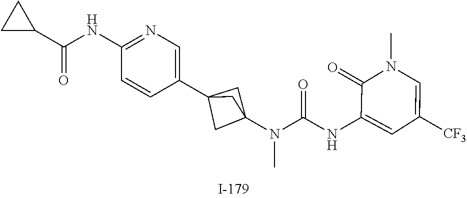 [SMILES: CN(C(=O)Nc1cc(C(F)(F)F)cn(C)c1=O)C12CC(c3ccc(NC(=O)C4CC4)nc3)(C1)C2]

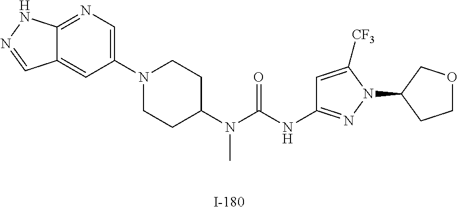 [SMILES: CN(C(=O)Nc1cc(C(F)(F)F)n([C@@H]2CCOC2)n1)C1CCN(c2cnc3[nH]ncc3c2)CC1]

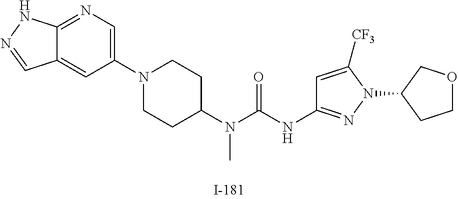 [SMILES: CN(C(=O)Nc1cc(C(F)(F)F)n([C@H]2CCOC2)n1)C1CCN(c2cnc3[nH]ncc3c2)CC1]

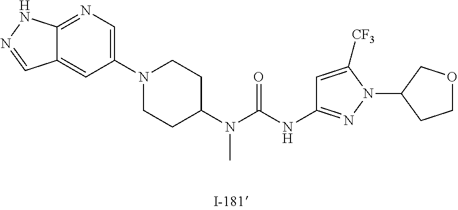 [SMILES: CN(C(=O)Nc1cc(C(F)(F)F)n(C2CCOC2)n1)C1CCN(c2cnc3[nH]ncc3c2)CC1]

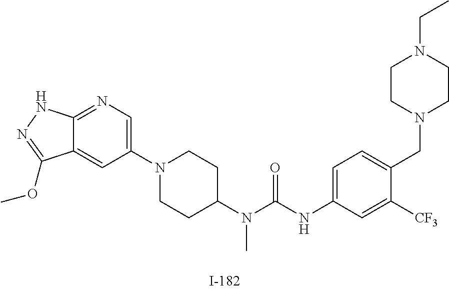 [SMILES: CCN1CCN(Cc2ccc(NC(=O)N(C)C3CCN(c4cnc5[nH]nc(OC)c5c4)CC3)cc2C(F)(F)F)CC1]

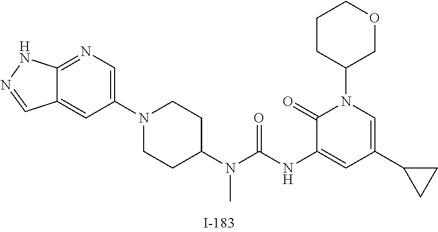 [SMILES: CN(C(=O)Nc1cc(C2CC2)cn(C2CCCOC2)c1=O)C1CCN(c2cnc3[nH]ncc3c2)CC1]

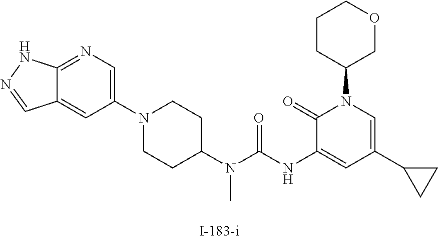 [SMILES: CN(C(=O)Nc1cc(C2CC2)cn([C@H]2CCCOC2)c1=O)C1CCN(c2cnc3[nH]ncc3c2)CC1]

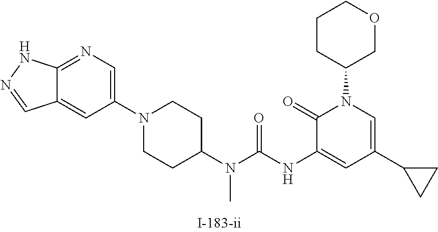 [SMILES: CN(C(=O)Nc1cc(C2CC2)cn([C@@H]2CCCOC2)c1=O)C1CCN(c2cnc3[nH]ncc3c2)CC1]

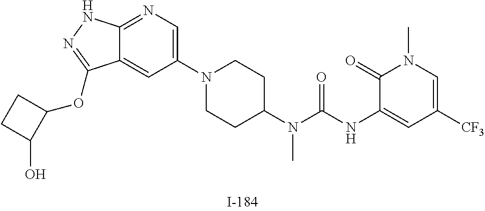 [SMILES: CN(C(=O)Nc1cc(C(F)(F)F)cn(C)c1=O)C1CCN(c2cnc3[nH]nc(OC4CCC4O)c3c2)CC1]

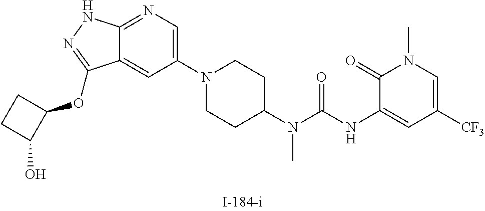 [SMILES: CN(C(=O)Nc1cc(C(F)(F)F)cn(C)c1=O)C1CCN(c2cnc3[nH]nc(O[C@@H]4CC[C@H]4O)c3c2)CC1]

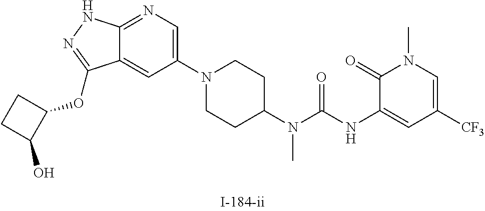 [SMILES: CN(C(=O)Nc1cc(C(F)(F)F)cn(C)c1=O)C1CCN(c2cnc3[nH]nc(O[C@H]4CC[C@@H]4O)c3c2)CC1]

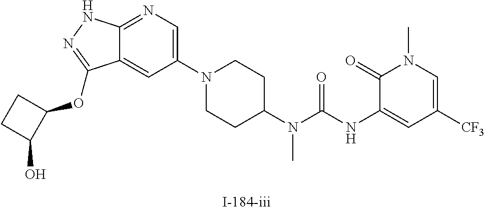 [SMILES: CN(C(=O)Nc1cc(C(F)(F)F)cn(C)c1=O)C1CCN(c2cnc3[nH]nc(O[C@@H]4CC[C@@H]4O)c3c2)CC1]

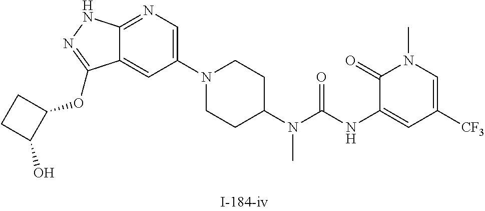 [SMILES: CN(C(=O)Nc1cc(C(F)(F)F)cn(C)c1=O)C1CCN(c2cnc3[nH]nc(O[C@H]4CC[C@H]4O)c3c2)CC1]

In some embodiments, the present disclosure encompasses the recognition that provided compounds display certain desirable characteristics, e.g., as compared to other known compounds. For example, in some embodiments, provided compounds are more potent in one or more biochemical or cellular assays (e.g., the JAK2 Binding Assay or SET2-pSTAT5 Cellular Assay described herein) and/or have one or more other characteristics that make them more suitable for drug development, such as better selectivity over other kinases and/or better ADME (absorption, distribution, metabolism, and excretion) properties including but not limited to better permeability, cytotoxicity, hepatocyte stability, solubility, and/or plasma protein binding profiles (e.g., based on assays described in the ensuing examples), than other known compounds. In some embodiments, provided compounds display certain desirable characteristics in one or more assays described herein, e.g., compared to other known compounds.

In some embodiments, provided compounds are provided and/or utilized in a salt form (e.g., a pharmaceutically acceptable salt form). Reference to a compound provided herein is understood to include reference to salts thereof, unless otherwise indicated. Pharmaceutically acceptable salt forms are known in the art. For example, S. M. Berge, et al. describes pharmaceutically acceptable salts in detail in J. Pharmaceutical Sciences, 66:1-19(1977).

It will be appreciated that throughout the present disclosure, unless otherwise indicated, reference to a compound of Formula I is intended to also include Formulae I, IA, IB, IC, ID, II, III, IV, and V and compound species of such formulas disclosed herein. It will also be appreciated that throughout the present disclosure, unless otherwise indicated, reference to a compound of Formula A is intended to also include Formulae B, C, D, E, I, IA, IB, IC, ID, II, III, IV, V, VI, VII, VIII, IX, X, and XI and compound species of such formulas disclosed herein.

### Preparing Provided Compounds

Provided compounds may generally be made by the processes described in the ensuing schemes and examples. In some embodiments, provided compounds (e.g., compounds of Formula I wherein X is —C(R⁶)₂—) are prepared according to the following Scheme:

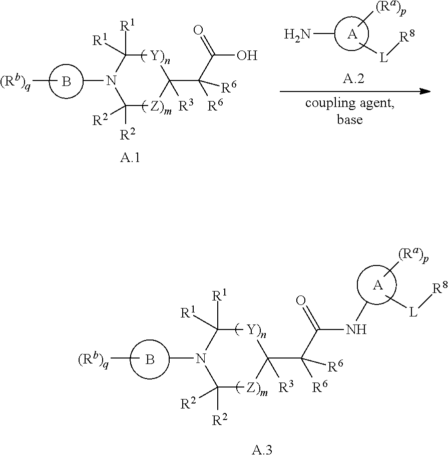

wherein Ring A, Ring B, L, m, n, p, q, R^{a}, R^{b}, R¹, R², R³, R⁶, R′, Y, and Z are as defined above for Formula I and described in classes and subclasses herein, both singly and in combination. Accordingly, in some embodiments, compound A.3 is prepared by a process comprising contacting intermediate A.1 with intermediate A.2 in the presence of a suitable coupling agent (e.g., HATU or POCl₃) and/or a suitable base (DIPEA or pyridine). In some embodiments, compound A.1 is provided in a protected form (e.g., wherein a substituent on Ring B is protected, e.g., with a suitable protecting group, such as Boc). In some embodiments, after coupling of intermediate A.1 with intermediate A.2, the resulting compound undergoes a deprotection step under suitable conditions (e.g., acidic conditions, such as TFA or HCl). In some embodiments, after a deprotection step, the resulting compound is further functionalized via an amide coupling reaction (such as with acetyl chloride or acetic anhydride).

In some embodiments, provided compounds (e.g., compounds of Formula I wherein X is —N(R⁷)—) are prepared according to the following Scheme:

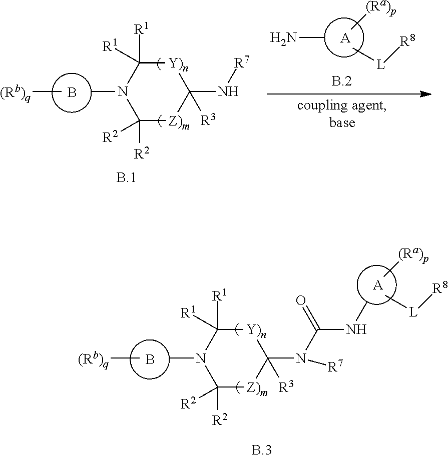

wherein Ring A, Ring B, L, m, n, p, q, R^{a}, R^{b}, R¹, R², R³, R⁷, R′, Y, and Z are as defined above for Formula I and described in classes and subclasses herein, both singly and in combination. Accordingly, in some embodiments, compound B.3 is prepared by a process comprising contacting intermediate B.1 with intermediate B.2 in the presence of a suitable coupling agent (e.g., carbonyl diimidazole or triphosgene) and/or a suitable base (DIPEA or triethylamine). In some embodiments, compound B.1 is provided in a protected form (e.g., wherein a nitrogen atom of Ring B is protected, e.g., with a suitable protecting group, such as SEM). In some embodiments, after coupling of intermediate B.1 with intermediate B.2, the resulting compound undergoes a deprotection step under suitable conditions (e.g., acidic conditions, such as TFA).

In some embodiments, provided compounds (e.g., compounds of Formula A wherein X is —N(R⁷)— or —O—) are prepared according to the following Scheme:

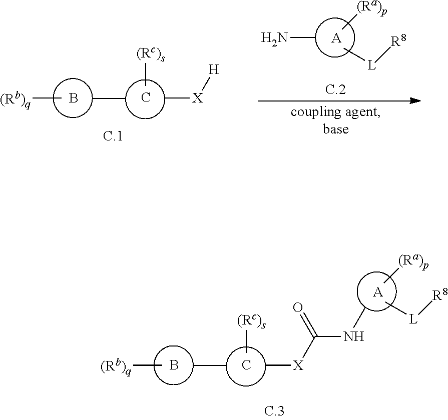

wherein Ring A, Ring B, Ring C, L, p, q, s, R^{a}, R^{b}, R^{c}, and R⁸ are as defined above for Formula I and described in classes and subclasses herein, both singly and in combination; and X is —N(R⁷)— or —O—. Accordingly, in some embodiments, compound C.3 is prepared by a process comprising contacting intermediate C.1 with intermediate C.2 in the presence of a suitable coupling agent (e.g., carbonyl diimidazole or triphosgene) and/or a suitable base (DIPEA or triethylamine). In some embodiments, compound C.1 is provided in a protected form (e.g., wherein a nitrogen atom of Ring B is protected, e.g., with a suitable protecting group, such as SEM). In some embodiments, after coupling of intermediate C.1 with intermediate C.2, the resulting compound undergoes a deprotection step under suitable conditions (e.g., acidic conditions, such as TFA).

In some embodiments, provided compounds (e.g., compounds of Formula A wherein X is —C(R⁶)₂—) are prepared according to the following Scheme:

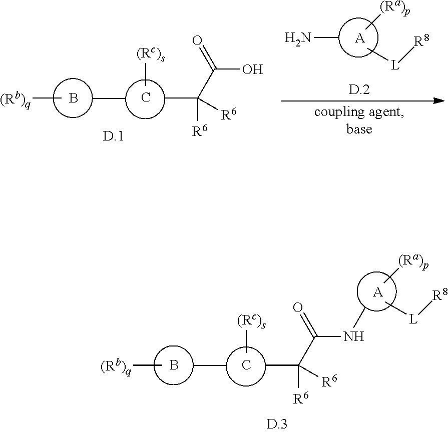

wherein Ring A, Ring B, Ring C, L, p, q, s, R^{a}, R^{b}, R^{c}, R⁶, and R⁸ are as defined above for Formula I and described in classes and subclasses herein, both singly and in combination. Accordingly, in some embodiments, compound D.3 is prepared by a process comprising contacting intermediate D.1 with intermediate D.2 in the presence of a suitable coupling agent (e.g., HATU or POCl₃) and/or a suitable base (DIPEA or pyridine). In some embodiments, compound D.1 is provided in a protected form (e.g., wherein a substituent on Ring B is protected, e.g., with a suitable protecting group, such as Boc). In some embodiments, after coupling of intermediate D.1 with intermediate D.2, the resulting compound undergoes a deprotection step under suitable conditions (e.g., acidic conditions, such as TFA or HCl). In some embodiments, after a deprotection step, the resulting compound is further functionalized via an amide coupling reaction (such as with acetyl chloride or acetic anhydride).

### Compositions

The present disclosure also provides compositions comprising a compound provided herein with one or more other components. In some embodiments, provided compositions comprise and/or deliver a compound described herein (e.g., compounds of Formulae A, B, C, D, E, I, IA, IB, IC, ID, II, III, IV, V, VI, VII, VIII, IX, X, and XI).

In some embodiments, a provided composition is a pharmaceutical composition that comprises and/or delivers a compound provided herein (e.g., compounds of Formulae A, B, C, D, E, I, IA, IB, IC, ID, II, III, IV, V, VI, VII, VIII, IX, X, and XI) and further comprises a pharmaceutically acceptable carrier. Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with JAK2.

In some embodiments, provided compounds are useful as JAK2 inhibitors. In some embodiments, provided compounds are useful as Type II JAK2 inhibitors. In some embodiments, the present disclosure provides methods of inhibiting JAK2 in a subject comprising administering a provided compound or composition. In some embodiments, the present disclosure provides methods of inhibiting JAK2 in a biological sample comprising contacting the sample with a provided compound or composition.

JAK (e.g., JAK2) has been implicated in various diseases, disorders, and conditions, such as myeloproliferative neoplasms (Vainchenker, W. et al., F1000Research 2018, 7(F1000 Faculty Rev):82), atopic dermatitis (Rodrigues, M. A. and Torres, T. J. Derm. Treat. 2019, 31(1), 33-40) and acute respiratory syndrome, hyperinflammation, and/or cytokine storm syndrome (The Lancet. doi:10.1016/S0140-6736(20)30628-0). Accordingly, in some embodiments, the present disclosure provides methods of treating a disease, disorder or condition associated with JAK2 in a subject in need thereof comprising administering to the subject a provided compound or composition. In some embodiments, a disease, disorder or condition is associated with overexpression of JAK2.

In some embodiments, the present disclosure provides methods of treating cancer, comprising administering a provided compound or composition to a subject in need thereof. In some embodiments, the present disclosure provides methods of treating proliferative diseases, comprising administering a provided compound or composition to a subject in need thereof.

In some embodiments, the present disclosure provides methods of treating a hematological malignancy, comprising administering a provided compound or composition to a subject in need thereof. In some embodiments, a hematological malignancy is leukemia (e.g., chronic lymphocytic leukemia, acute lymphoblastic leukemia, T-cell acute lymphoblastic leukemia, chronic myelogenous leukemia, acute myelogenous leukemia, or acute monocytic leukemia). In some embodiments, a hematological malignancy is lymphoma (e.g., Burkitt's lymphoma, Hodgkin's lymphoma, or non-Hodgkin's lymphoma). In some embodiments, a non-Hodgkin's lymphoma is a B-cell lymphoma. In some embodiments, a non-Hodgkin's lymphoma is a NK/T-cell lymphoma (e.g., cutaneous T-cell lymphoma). In some embodiments, a hematological malignancy is myeloma (e.g., multiple myeloma). In some embodiments, a hematological malignancy is a myeloproliferative neoplasm (e.g., polycythemia vera, essential thrombocytopenia, or myelofibrosis). In some embodiments, a hematological malignancy is myelodysplastic syndrome.

In some embodiments, the present disclosure provides methods of treating an inflammatory disease, disorder, or condition (e.g., acute respiratory syndrome, hyperinflammation, and/or cytokine storm syndrome (including those associated with COVID-19) or atopic dermatitis), comprising administering a provided compound or composition to a subject in need thereof.

In some embodiments, a provided compound or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a provided compound or composition is administered to a subject who is receiving or has received one or more additional therapies (e.g., an anti-cancer therapy and/or therapy to address one or more side effects of such anti-cancer therapy, or otherwise to provide palliative care). Exemplary additional therapies include BCL2 inhibitors (e.g., venetoclax), HDAC inhibitors (e.g., vorinostat), BET inhibitors (e.g., mivebresib), proteasome inhibitors (e.g., bortezomib), LSD1 inhibitors (e.g., IMG-7289), and CXCR2 inhibitors. Useful combinations of a JAK2 inhibitor with BCL2, HDAC, BET, and proteasome inhibitors have been demonstrated in cells derived from cutaneous T-cell lymphoma patients (Yumeen, S., et al., Blood Adv. 2020, 4(10), 2213-2226). A combination of a JAK2 inhibitor with a LSD1 inhibitor demonstrated good efficacy in a mouse model of myeloproliferative neoplasms (Jutzi, J. S., et al., HemaSphere 2018, 2(3), http://dx.doi.org/10.1097/HS9.0000000000000054). CXCR2 activity has been shown to modulate signaling pathways involved in tumor growth, angiogenesis, and/or metastasis, including the JAK-STAT3 pathway (Jaffer, T., Ma, D. Transl. Cancer Res. 2016, 5(Suppl. 4), S616-S628).

### Exemplary Embodiments

The following numbered embodiments, while non-limiting, are exemplary of certain aspects of the present disclosure:
1. A compound of Formula I:

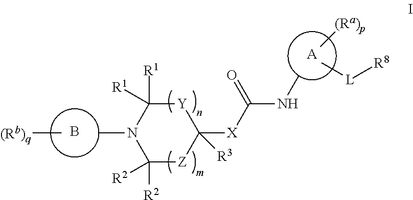

or a pharmaceutically acceptable salt thereof, wherein:
X is —C(R⁶)₂— or —N(R⁷)—;
each Y is —C(R⁴)₂—;
each Z is —C(R⁵)₂—;
n is 0, 1, or 2;
m is 0, 1, or 2,
   provided that at least one of n and m is 1 or 2;
each R¹, R², R³, R⁴, and R⁵ is independently hydrogen, halogen, —CN, or optionally substituted C₁₋₆ aliphatic, and/or
   two R¹ groups and/or two R² groups and/or two R⁴ groups and/or two R⁵ groups, together with the atom(s) to which they are attached, combine to form a 3- to 8-membered saturated or partially unsaturated ring, and/or
   two R¹ groups and/or two R² groups and/or two R⁴ groups and/or two R⁵ groups, together with the atom to which they are attached, combine to form an oxo, and/or
   a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group, together with the atoms to which they are attached, combine to form a 3- to 8-membered saturated or partially unsaturated ring;
each R⁶ is independently hydrogen or optionally substituted C₁₋₆ aliphatic, or
   two R⁶ groups, together with the atom to which they are attached, combine to form a 3- to 8-membered saturated or partially unsaturated ring;
R⁷ is hydrogen or optionally substituted C₁₋₆ aliphatic;
Ring A is an optionally substituted group selected from phenyl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur;
Ring B is an optionally substituted group selected from 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, or 10- to 16-membered polycyclic heteroaryl having 1-5 heteroatoms independently selected from nitrogen, oxygen, and sulfur;
L is a covalent bond or a bivalent C₁₋₃ straight or branched hydrocarbon chain;
R⁸ is hydrogen, halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur;
each R^{a} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
each R^{b} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
p is 0, 1, 2, 3, 4, or 5, as valency permits;
q is 0, 1, 2, 3, 4, or 5, as valency permits;
each R is independently hydrogen or an optionally substituted group selected from C₁₋₆ aliphatic, C₃₋₇ cycloaliphatic, 3- to 7-membered saturated or partially unsaturated monocyclic heterocyclyl having 1-3 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
   two R, when attached to the same nitrogen atom, are taken together to form an optionally substituted 3- to 7-membered saturated or partially unsaturated ring having 0-1 additional heteroatoms independently selected from nitrogen, oxygen, and sulfur; and
each R′ is independently an optionally substituted group selected from C₁₋₆ aliphatic and C₃₋₇ cycloaliphatic.
2. The compound of embodiment 1, wherein Ring B is an optionally substituted group selected from 5- to 6-membered monocyclic heteroaryl having 1-4 heteroatoms independently selected from nitrogen, oxygen, and sulfur and 8- to 10-membered bicyclic heteroaryl having 1-4 heteroatoms independently selected from nitrogen, oxygen, and sulfur.
3. The compound of embodiment 1 or 2, wherein the moiety

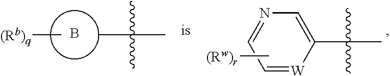

wherein:
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
two R^{w} groups, together with the atoms to which they are attached, combine to form an 5- to 6-membered partially unsaturated or aromatic ring substituted with q R^{b} groups; and
r is 0, 1, 2, or 3.
4. The compound of embodiment 3, wherein W is CH.
5. The compound of embodiment 3, wherein W is N.
6. The compound of any one of embodiments 1-5, wherein r is 1.
7. The compound of embodiment 6, wherein R^{w} is independently —N(R)C(O)R′ or —N(R)C(O)N(R)₂.
8. The compound of any one of embodiments 1-5, wherein r is 2.
9. The compound of embodiment 8, wherein two R^{w} groups, together with the atoms to which they are attached, combine to form an 5- to 6-membered partially unsaturated or aromatic ring substituted with q R^{b} groups.
10. The compound of embodiment 9, wherein each R^{b} is independently halogen, —N(R)₂, optionally substituted C₁₋₆ aliphatic, or optionally substituted C₃₋₆ cycloaliphatic.
11. The compound of any one of embodiments 1-10, wherein the compound is not:

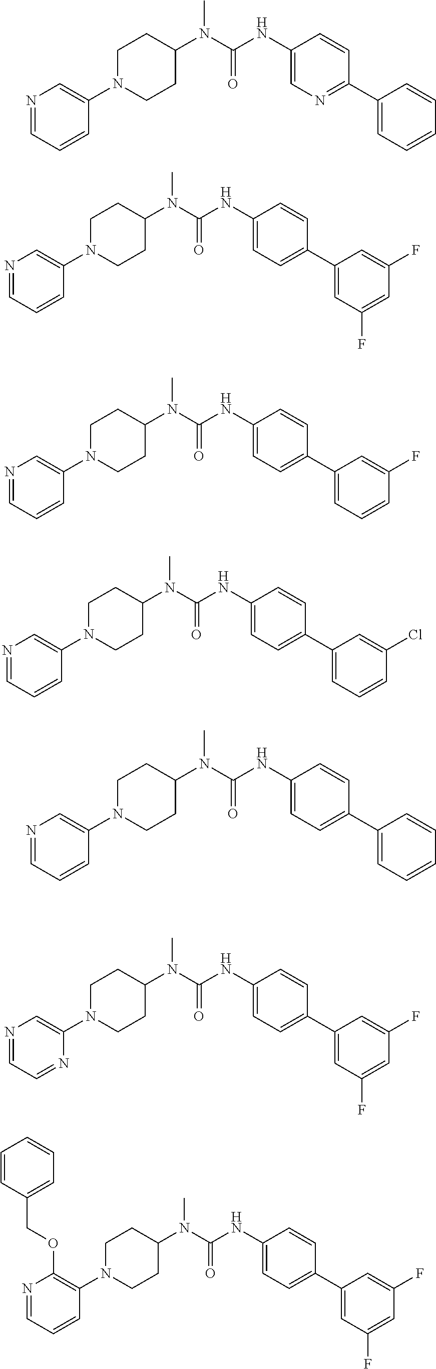

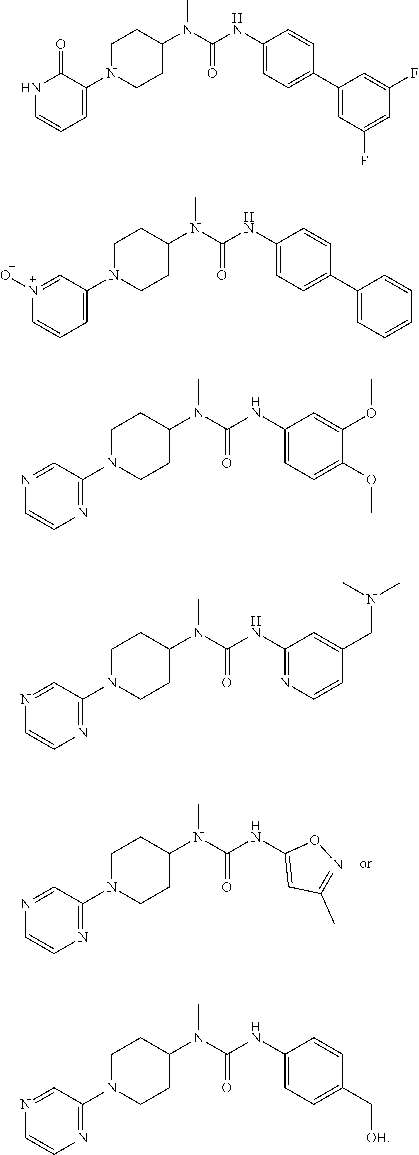

12. The compound of embodiment 1 or 2, wherein the moiety

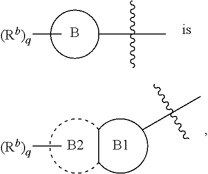

wherein:
Ring B1 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur.
13. The compound of embodiment 12, wherein Ring B1 is a 5- to 6-membered monocyclic heteroaryl having 1-3 heteroatoms independently selected from nitrogen, oxygen, and sulfur.
14. The compound of embodiment 13, wherein Ring B1 is a 5-membered monocyclic heteroaryl having 1-3 heteroatoms independently selected from nitrogen, oxygen, and sulfur.
15. The compound of embodiment 13, wherein Ring B1 is a 6-membered monocyclic heteroaryl having 1-3 heteroatoms independently selected from nitrogen, oxygen, and sulfur.
16. The compound of any one of embodiments 12-15, wherein Ring B1 is fused to Ring B2.
17. The compound of any one of embodiments 12-16, wherein Ring B2 is a 5- to 6-membered monocyclic heteroaryl having 1-3 heteroatoms independently selected from nitrogen, oxygen, and sulfur.
18. The compound of any one of embodiments 12-15, wherein Ring B1 is not fused to Ring B2.
19. The compound of any one of embodiments 1-18, wherein the compound is not:

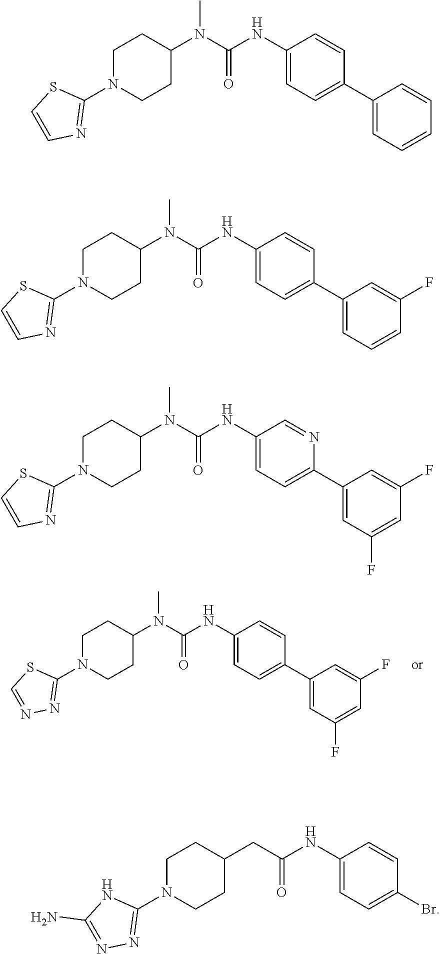 [SMILES: CN(C(=O)Nc1ccc(-c2ccccc2)cc1)C1CCN(c2nccs2)CC1 ; CN(C(=O)Nc1ccc(-c2cccc(F)c2)cc1)C1CCN(c2nccs2)CC1 ; CN(C(=O)Nc1ccc(-c2cc(F)cc(F)c2)nc1)C1CCN(c2nccs2)CC1 ; CN(C(=O)Nc1ccc(-c2cc(F)cc(F)c2)cc1)C1CCN(c2nncs2)CC1 ; Nc1nnc(N2CCC(CC(=O)Nc3ccc(Br)cc3)CC2)[nH]1]

20. The compound of any one of embodiments 1-19, wherein a moiety

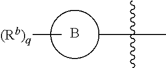

is:
(i)

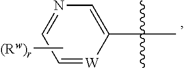

wherein:
W is CH, CR^{w}, or N;
each R′ is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
two R^{w} groups, together with the atoms to which they are attached, combine to form an 5- to 6-membered partially unsaturated or aromatic ring substituted with q R^{b} groups; and
r is 0, 1, 2, or 3; or
(ii)

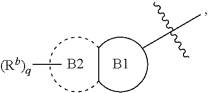

wherein:
Ring B1 is a 5-membered monocyclic heteroaryl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur.
21. The compound of any one of embodiments 1-20, wherein each R^{b} is independently halogen, —OR, —N(R)₂, —N(R)C(O)R′, —N(R)C(O)N(R)₂, optionally substituted C₁₋₆ aliphatic, or optionally substituted C₃₋₆ cycloaliphatic.
22. The compound of any one of embodiments 1-21, wherein q is 0 or 1.
23. The compound of any one of embodiments 1-22, wherein X is —C(R⁶)₂—.
24. The compound of any one of embodiments 1-23, wherein each R⁶ is hydrogen.
25. The compound of any one of embodiments 1-23, wherein two R⁶ groups, together with the atom to which they are attached, combine to form a 3- to 8-membered saturated or partially unsaturated ring.
26. The compound of any one of embodiments 1-22, wherein X is —N(R⁷)—.
27. The compound of any one of embodiments 1-22 and 26, wherein R⁷ is optionally substituted C₁₋₆ aliphatic.
28. The compound of embodiment 27, wherein R⁷ is C₁₋₆ alkyl.
29. The compound of any one of embodiments 1-28, wherein n is 1.
30. The compound of any one of embodiments 1-29, wherein m is 1.
31. The compound of any one of embodiments 1-30, wherein each R¹, R², R³, R⁴, and R⁵ is independently hydrogen, halogen, —CN, or optionally substituted C₁₋₆ aliphatic.
32. The compound of embodiment 31, wherein each R¹, R², R³, and R⁴ is hydrogen, and each R⁵ is independently hydrogen, halogen, —CN, or optionally substituted C₁₋₆ aliphatic.
33. The compound of embodiment 32, wherein each R¹, R², R³, R⁴, and R⁵ is hydrogen.
34. The compound of any one of embodiments 1-30, wherein one of the following occurs:
   (i) two R¹ groups, together with the atom(s) to which they are attached, combine to form a 3- to 8-membered saturated or partially unsaturated ring;
   (ii) two R² groups, together with the atom(s) to which they are attached, combine to form a 3- to 8-membered saturated or partially unsaturated ring;
   (iii) two R⁴ groups, together with the atom(s) to which they are attached, combine to form a 3- to 8-membered saturated or partially unsaturated ring; and
   (iv) two R⁵ groups, together with the atom(s) to which they are attached, combine to form a 3- to 8-membered saturated or partially unsaturated ring.
35. The compound of any one of embodiments 1-30 and 34, wherein one of the following occurs:
   (i) two R¹ groups, together with the atom to which they are attached, combine to form an oxo;
   (ii) two R² groups, together with the atom to which they are attached, combine to form an oxo;
   (iii) two R⁴ groups, together with the atom to which they are attached, combine to form an oxo; and
   (iv) two R⁵ groups, together with the atom to which they are attached, combine to form an oxo.
36. The compound of any one of embodiments 1-30 and 34-35, wherein one of the following occurs:
   (i) a R¹ and R² group, together with the atoms to which they are attached, combine to form a 3- to 8-membered saturated or partially unsaturated ring;
   (ii) a R¹ and R³ group, together with the atoms to which they are attached, combine to form a 3- to 8-membered saturated or partially unsaturated ring;
   (iii) a R¹ and R⁴ group, together with the atoms to which they are attached, combine to form a 3- to 8-membered saturated or partially unsaturated ring;
   (iv) a R¹ and R⁵ group, together with the atoms to which they are attached, combine to form a 3- to 8-membered saturated or partially unsaturated ring;
   (v) a R² and R³ group, together with the atoms to which they are attached, combine to form a 3- to 8-membered saturated or partially unsaturated ring;
   (vi) a R² and R⁴ group, together with the atoms to which they are attached, combine to form a 3- to 8-membered saturated or partially unsaturated ring;
   (vii) a R² and R⁵ group, together with the atoms to which they are attached, combine to form a 3- to 8-membered saturated or partially unsaturated ring;
   (viii) a R³ and R⁴ group, together with the atoms to which they are attached, combine to form a 3- to 8-membered saturated or partially unsaturated ring;
   (ix) a R³ and R⁵ group, together with the atoms to which they are attached, combine to form a 3- to 8-membered saturated or partially unsaturated ring;
   (x) a R³ and R⁶ group, together with the atoms to which they are attached, combine to form a 3- to 8-membered saturated or partially unsaturated ring;
   (xi) a R³ and R⁷ group, together with the atoms to which they are attached, combine to form a 3- to 8-membered saturated or partially unsaturated ring; and
   (xii) a R⁴ and R⁵ group, together with the atoms to which they are attached, combine to form a 3- to 8-membered saturated or partially unsaturated ring.
37. The compound of any one of embodiments 1-36, wherein Ring A is phenyl or 5- to 6-membered monocyclic heteroaryl having 1-4 heteroatoms independently selected from nitrogen, oxygen, and sulfur.
38. The compound of embodiment 37, wherein Ring A is phenyl.
39. The compound of embodiment 37, wherein Ring A is 5- to 6-membered monocyclic heteroaryl having 1-4 heteroatoms independently selected from nitrogen, oxygen, and sulfur.
40. The compound of any one of embodiments 1-39, wherein L is a covalent bond.
41. The compound of any one of embodiments 1-39, wherein L is —CH₂—.
42. The compound of any one of embodiments 1-41, wherein R⁸ is halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur.
43. The compound of embodiment 42, wherein R⁸ is optionally substituted C₁₋₆ aliphatic or optionally substituted 3- to 6-membered saturated or partially unsaturated monocyclic heterocyclyl having 1-3 heteroatoms independently selected from nitrogen, oxygen, and sulfur.
44. The compound of any one of embodiments 1-43, wherein each R^{a} is independently halogen, —CN, —OR, —O(CH₂)₁₋₄R, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.
45. The compound of embodiment 44, wherein each R^{a} is optionally substituted C₁₋₆ aliphatic.
46. The compound of embodiment 45, wherein each R^{a} is C₁₋₆ alkyl optionally substituted with one or more halogen (e.g., fluoro).
47. The compound of any one of embodiments 1-46, wherein p is 0 or 1.
48. The compound of any one of embodiments 1-47, wherein a moiety

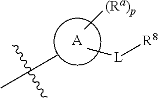

is selected from:

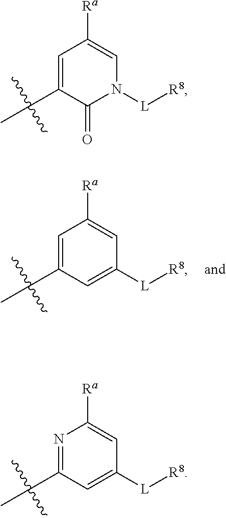

49. The compound of any one of embodiments 1-48, wherein at least one of R^{a} and

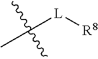

is C₁₋₄ haloalkyl.
50. The compound of any one of embodiments 1-49, wherein the compound is not:

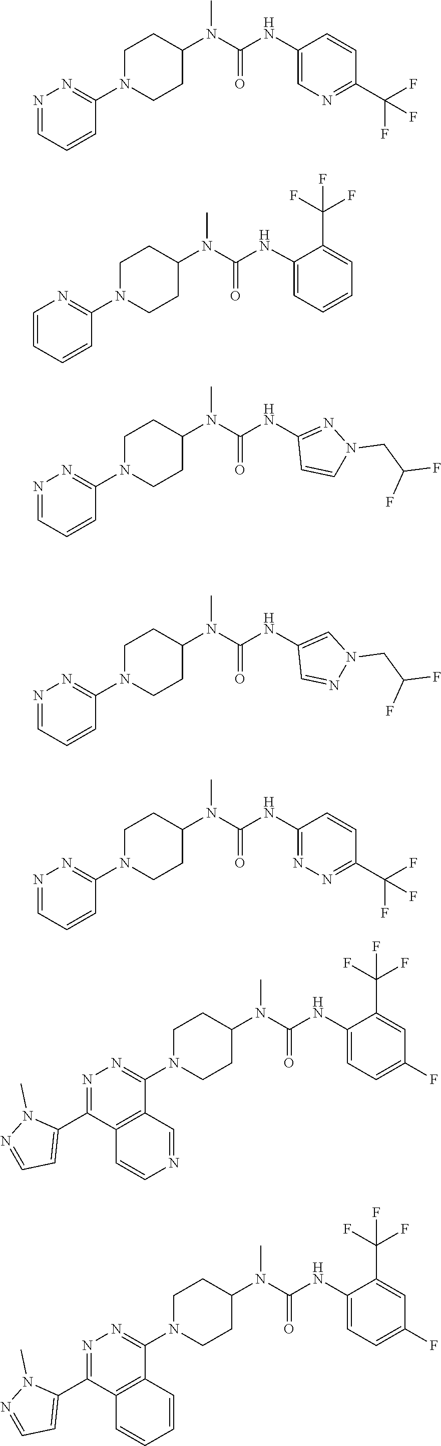

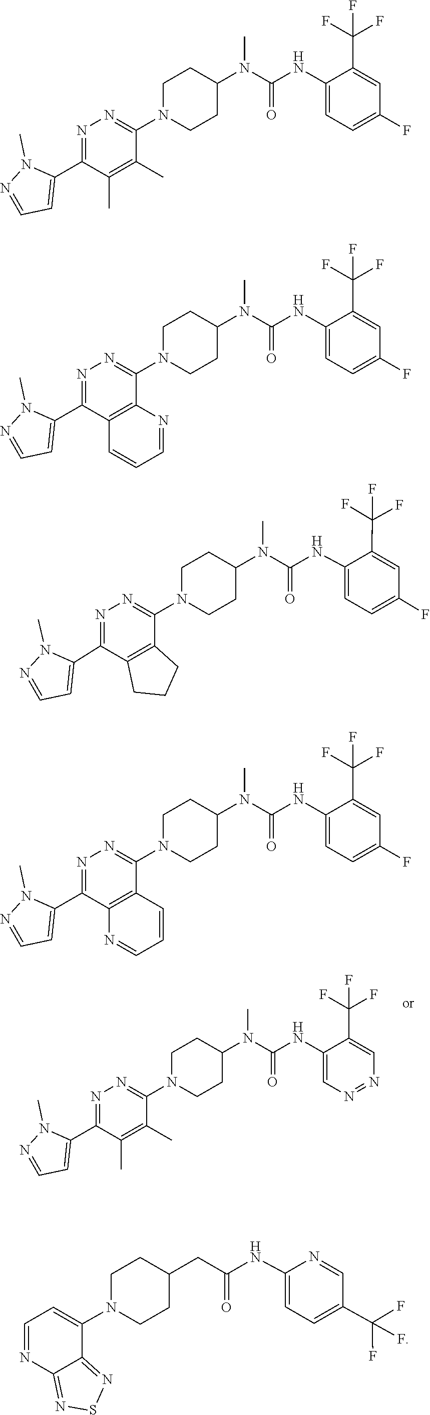 [SMILES: Cc1c(-c2ccnn2C)nnc(N2CCC(N(C)C(=O)Nc3ccc(F)cc3C(F)(F)F)CC2)c1C ; CN(C(=O)Nc1ccc(F)cc1C(F)(F)F)C1CCN(c2nnc(-c3ccnn3C)c3cccnc23)CC1 ; CN(C(=O)Nc1ccc(F)cc1C(F)(F)F)C1CCN(c2nnc(-c3ccnn3C)c3c2CCC3)CC1 ; CN(C(=O)Nc1ccc(F)cc1C(F)(F)F)C1CCN(c2nnc(-c3ccnn3C)c3ncccc23)CC1 ; Cc1c(-c2ccnn2C)nnc(N2CCC(N(C)C(=O)Nc3cnncc3C(F)(F)F)CC2)c1C ; O=C(CC1CCN(c2ccnc3nsnc23)CC1)Nc1ccc(C(F)(F)F)cn1]

51. The compound of any one of embodiments 1-50, wherein when R⁸ is hydrogen, then p is not 0.
52. The compound of any one of embodiments 1-51, wherein the compound is of Formula IA:

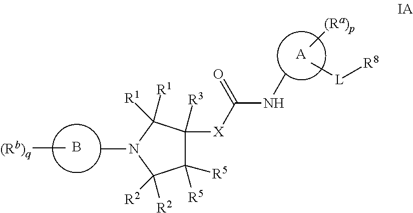

or a pharmaceutically acceptable salt thereof.
53. The compound of any one of embodiments 1-51, wherein the compound is of Formula

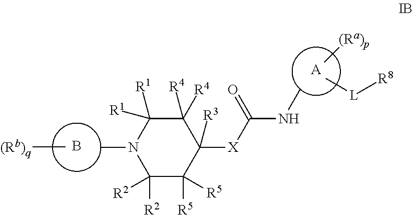

or a pharmaceutically acceptable salt thereof.
54. The compound of any one of embodiments 1-51, wherein the compound is of Formula IC:

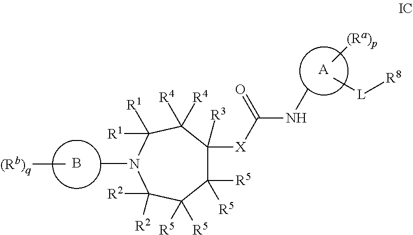

or a pharmaceutically acceptable salt thereof.
55. The compound of any one of embodiments 1-51, wherein the compound is of Formula ID:

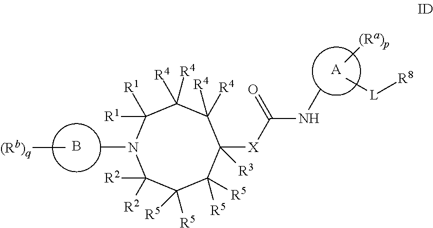

or a pharmaceutically acceptable salt thereof.
56. The compound of any one of embodiments 1-55, wherein the compound is of Formula II:

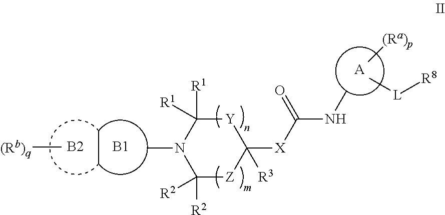

or a pharmaceutically acceptable salt thereof, wherein:
Ring B1 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur, wherein Ring B1 is optionally fused to Ring B2; and
Ring B2, when present, is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur,
wherein at least one of Ring B1 and Ring B2 is aromatic; and
wherein at least one of Ring B1 and Ring B2 contains a heteroatom.
57. The compound of any one of embodiments 1-55, wherein the compound is of Formula III:

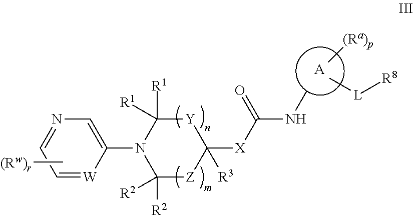

or a pharmaceutically acceptable salt thereof, wherein:
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
   two R^{w} groups, together with the atoms to which they are attached, combine to form a 5-to 6-membered partially unsaturated or aromatic ring substituted with 0-4 R^{b} groups; and
r is 0, 1, 2, or 3.
58. The compound of any one of embodiments 1-57, wherein the compound is of Formula IV:

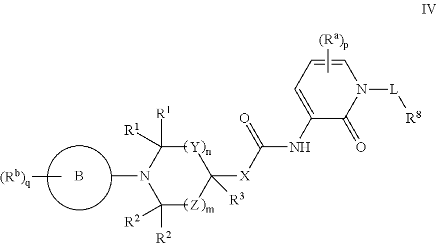

or a pharmaceutically acceptable salt thereof.
59. The compound of any one of embodiments 1-57, wherein the compound is of Formula V:

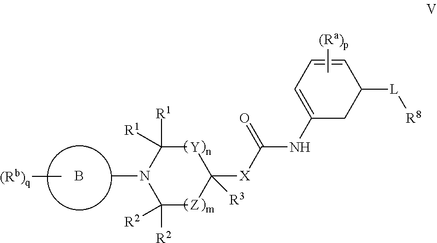

or a pharmaceutically acceptable salt thereof.
60. A compound selected from Table 1, or a pharmaceutically acceptable salt thereof.
61. A pharmaceutical composition comprising a compound of any one of the preceding embodiments, or a pharmaceutically acceptable salt thereof, and a pharmaceutically acceptable carrier.
62. A method of inhibiting JAK2 in a subject comprising administering the compound of any one of embodiments 1-60 or the composition of embodiment 61.
63. A method of treating a disease, disorder, or condition associated with JAK2, comprising administering to a subject in need thereof the compound of any one of embodiments 1-60 or the composition of embodiment 61.
64. A method of treating cancer, comprising administering to a subject in need thereof the compound of any one of embodiments 1-60 or the composition of embodiment 61.
65. A method of treating a hematological malignancy, comprising administering to a subject in need thereof the compound of any one of embodiments 1-60 or the composition of embodiment 61.
66. The method of embodiment 65, wherein the hematological malignancy is leukemia or lymphoma.
67. A method of treating a myeloproliferative neoplasm, comprising administering to a subject in need thereof the compound of any one of embodiments 1-60 or the composition of embodiment 61.
68. The method of embodiment 67, wherein the myeloproliferative neoplasm is polycythemia vera, essential thrombocytopenia or myelofibrosis.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Chiral HPLC and SFC Methods

The following exemplary chiral HPLC and SFC methods were used in the synthesis of provided compounds, as indicated below:

Method A: column: CHIRALCEL OX-H (250 mm*20 mm, 5 μm); mobile phase: (A) liquid CO₂, (B) 0.1% diethylamine (DEA) in propan-2-ol: methanol (50:50); flow rate: 80 mL/min.

Method B: column: CHIRALCEL OJ-H (250 mm*21 mm, 5 μm); mobile phase: (A) liquid CO₂, (B) 0.1% DEA in in propan-2-ol: MeCN (50:50); flow rate: 80 mL/min.

Method C: column: CHIRALPAK IG (250 mm*21 mm, 5 μm); mobile phase: (A) 0.1% DEA in n-hexane, (B) 0.1% DEA in propan-2-ol: MeCN (70:30); flow rate: 20 mL/min.

Method D: column: CHIRALCEL IC (250 mm*21 mm, 5 μm); mobile phase: (A) liquid CO₂, (B) 0.1% DEA in in propan-2-ol: MeCN (50:50); flow rate: 80 mL/min.

Method E: column: CHIRALPAK IC (250 mm*21 mm, 5 μm); mobile phase: (A) 0.1% DEA in n-hexane, (B) 0.1% DEA in propan-2-ol: MeCN (70:30); flow rate: 20 mL/min.

Method F: column: CHIRALPAK IG (250 mm*21 mm, 5 μm); mobile phase: (A) Liquid CO₂, (B) 0.1% DEA in propan-2-ol: MeCN (50:50); flow rate: 80 mL/min.

Method G: column: CHIRALPAK IH (250 mm*21 mm, 5 μm); mobile phase: (A) 0.1% DEA in n-hexane, (B) 0.1% DEA in propan-2-ol: MeCN (50:50); flow rate: 20 mL/min.

Method H: column: CHIRALPAK AD-H (250 mm*21 mm, 5 μm); mobile phase: (A) liquid CO₂, (B) 0.1% DEA in propan-2-ol: MeCN (50:50); flow rate: 80 mL/min.

Method I: column: CHIRALPAK IH (250 mm*21 mm, 5 μm); mobile phase: (A) liquid CO₂, (B) 0.1% DEA in propan-2-ol: MeCN (50:50); flow rate: 80 mL/min

Method J: CHIRALPAK IB-N (250 mm*21 mm, 5 μm); mobile phase: (A) liquid CO₂, (B) 0.1% DEA in in propan-2-ol: MeCN (50:50); flow rate: 80 mL min.

Method K: CHIRALPAK IB-N (250 mm*21 mm, 5 μm); mobile phase: (A) 0.1% DEA in n-hexane, (B) 0.1% DEA in propan-2-ol: MeCN (70:30); flow rate: 20 mL/min.

Method L: column: CHIRALCEL OX-H (250 mm*20 mm, 5 μm); mobile phase: (A) 0.1% DEA in n-hexane, (B) 0.1% DEA in propane-2-ol: MeCN (70:30); flow rate: 20 mL/min.

Method M: column: CHIRALPAK AD-H (250 mm*21 mm, 5 um); mobile phase: (A) 0.1% DEA in n-hexane, (B) 0.1% DEA in propane-2-ol: MeCN (50:50); flow rate: 20 mL/min.

### Preparation of Intermediates

### Preparation of Intermediate H-1: 2-bromo-5-((2-(trimethylsilyl)ethoxy)methyl)-5H-pyrrolo[2,3-b]pyrazine

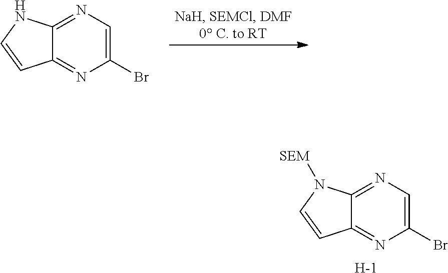

Synthesis of compound H-1. To a solution of 2-bromo-5H-pyrrolo[2,3-b]pyrazine (1 g, 5 mmol, 1.0 equiv) in DMF (10 mL) was added sodium hydride (0.728 g. 15 mmol, 3 equiv) at 0° C. and stirred for 45 min. To the mixture was added 2-(trimethylsilyl)ethoxymethyl chloride (1.25 g, 7.5 mmol, 1.5 equiv) dropwise and stirred at room temperature for 1 h. The reaction mixture was quenched with cold water carefully and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford H-1 (1 g, 60%). MS (ES): m/z 328.1 and 330.1 [M+H]⁺.

The following intermediates were prepared following the procedure of H-1:

| ID | Structure | Name | MS (ES): m/z |
|---|---|---|---|
| H-2 | 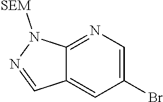 [SMILES: C[Si](C)(C)CCOCn1ncc2cc(Br)cnc21] | 5-bromo-1-((2-(trimethylsilyl)ethoxy)methyl)-1H- pyrazolo[3,4-b]pyridine | 328.1 and 330.1 [M + H]⁺ |
| H-3 | 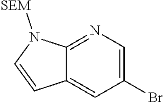 [SMILES: C[Si](C)(C)CCOCn1ccc2cc(Br)cnc21] | 5-bromo-1-((2-(trimethylsilyl)ethoxy)methyl)-1H- pyrrolo[2,3-b]pyridine | 327.1 and 329.1 [M + H]⁺ |
| H-5 | 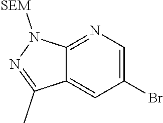 [SMILES: Cc1nn(COCC[Si](C)(C)C)c2ncc(Br)cc12] | 5-bromo-3-methyl-1-((2- (trimethylsilyl)ethoxy)methyl)-1H-pyrazolo[3,4- b]pyridine | 342.2 and 344.3 [M + H]⁺ |
| H-12 | 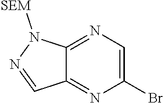 [SMILES: C[Si](C)(C)CCOCn1ncc2nc(Br)cnc21] | 5-bromo-1-((2-(trimethylsilyl)ethoxy)methyl)-1H- pyrazolo[3,4-b]pyrazine | 329.1 and 331.1 [M + H]⁺ |
| H-14 | 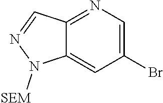 [SMILES: C[Si](C)(C)CCOCn1ncc2ncc(Br)cc21] | 6-bromo-1-((2-(trimethylsilyl)ethoxy)methyl)-1H- pyrazolo[4,3-b]pyridine | 328.1 and 330.1 [M + H]⁺ |

### Preparation of Intermediate H-4: 2-bromo-6-cyclopropyl-5-((2-(trimethylsilyl)ethoxy)methyl)-5H-pyrrolo[2,3-b]pyrazine

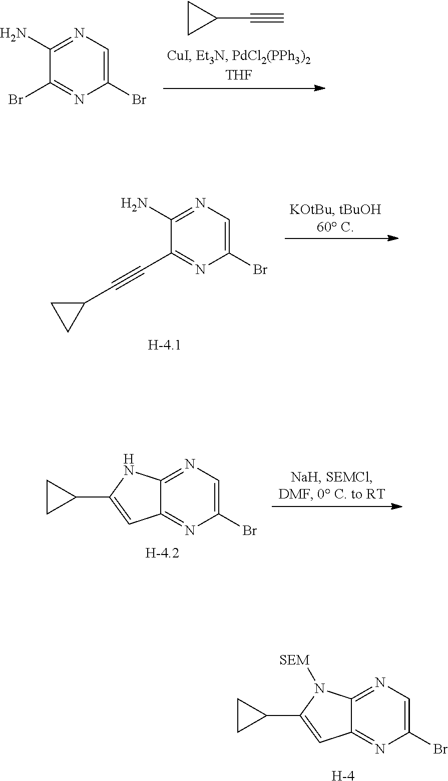

Synthesis of compound H-4.1. A mixture of 3,5-dibromopyrazin-2-amine (3.0 g, 11.86 mmol, 1.0 equiv), triethylamine (4.95 mL, 35.35 mmol, 3.0 equiv) and copper iodide (0.225 g, 1.185 mmol, 0.1 equiv) in THE (60 mL) was degassed by bubbling through a stream of argon for 5 min. Palladium(II)bis(triphenylphosphine) dichloride (0.866 g, 1.185 mmol, 0.1 equiv) and ethynylcyclopropane (0.822 gm, 12.45 mmol, 1.05 equiv) were added and degassed with argon for another 5 min. The reaction mixture was stirred at room temperature for 3 h. The reaction mixture was poured in water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 8% ethyl acetate in hexane) to afford H-4.1 (1.2 g, 42.5%). MS(ES): m/z 238.03 and 239.80 [M+H]⁺.

Synthesis of compound H-4.2. To a solution of H-4.1 (1.2 g, 5.04 mmol, 1.0 equiv) in tert-butanol (15 mL) was added potassium tert-butoxide (1.24 g, 11.09 mmol, 2.2 equiv). The reaction mixture was stirred at 60° C. for 2 h. It was poured into an aqueous 5 N hydrogen chloride solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford H-4.2 (0.90 g, 75%). MS(ES): m/z 238.12 and 240.1 [M+H]⁺.

Synthesis of compound H-4. Compound H-4 was prepared from H-4.2 following the procedure described in the synthesis of H-1. The product was purified by flash column chromatography on silica gel (CombiFlash®, 7.7% ethyl acetate in hexane). MS(ES): m/z 368.20 and 369.5 [M+H]⁺.

### Preparation of Intermediate H-6: 2-bromo-7-methyl-5-((2-(trimethylsilyl)ethoxy)methyl)-5H-pyrrolo[2,3-b]pyrazine

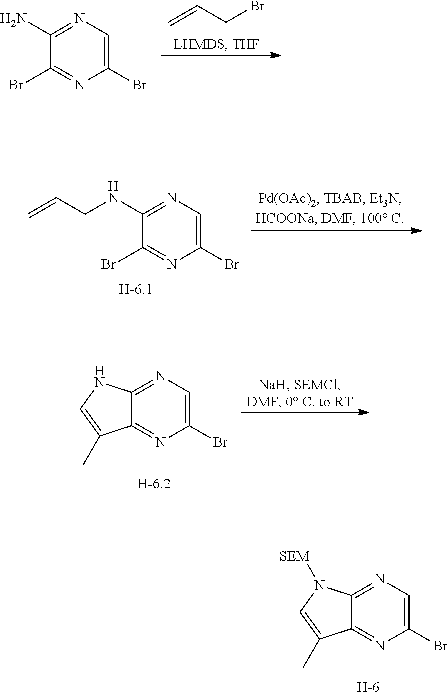

Synthesis of compound H-6.1. To a solution of 3,5-dibromopyrazin-2-amine (29 g, 114.2 mmol, 1.0 equiv) in THE (100 mL) was added a solution of lithium bis(trimethylsilyl)amide (1 M in THF, 140 mL, 142.0 mmol, 1.2 equiv) at room temperature and stirred for 2 h. To the solution was added allyl bromide (27.9 g, 229.0 mmol, 2.0 equiv) dropwise and stirred for 16 h. The reaction mixture was slowly quenched with an aqueous ammonium chloride solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.0% ethyl acetate in hexane) to afford H-6.1 (18 g, 54%). MS (ES): m/z 293.91 and 295.91 [M+H]⁺.

Synthesis of compound H-6.2. A mixture of H-6.1 (6 g, 3.1 mmol, 1 equiv), triethylamine (5.188 g, 42.8 mmol, 2.5 equiv), sodium formate (0.279 g, 4.10 mmol, 0.2 equiv), palladium (II) acetate (0.460 g, 2.0 mmol, 0.1 equiv) and tetrabutylammonium bromide (0.992 g, 3.0 mmol, 0.15 equiv) in DMF (6 mL) was purged with argon. The flask was sealed and reaction mixture was stirred at 100° C. for 16 h. It was cooled to room temperature, poured into water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 11% ethyl acetate in hexane) to afford H-6.2 (0.620 g, 14%). MS (ES): m/z 212.03 and 214.03 [M+H]⁺.

Synthesis of compound H-6. Compound H-6 was prepared from H-6.2 following the procedure described in the synthesis of H-1. The product was purified by flash column chromatography on silica gel (CombiFlash®, 10% ethyl acetate in hexane). MS (ES): m/z 342.2 and 344.24 [M+H]⁺.

### Preparation of Intermediate H-7: 2-bromo-6-methyl-5-((2-(trimethylsilyl)ethoxy)methyl)-5H-pyrrolo[2,3-b]pyrazine

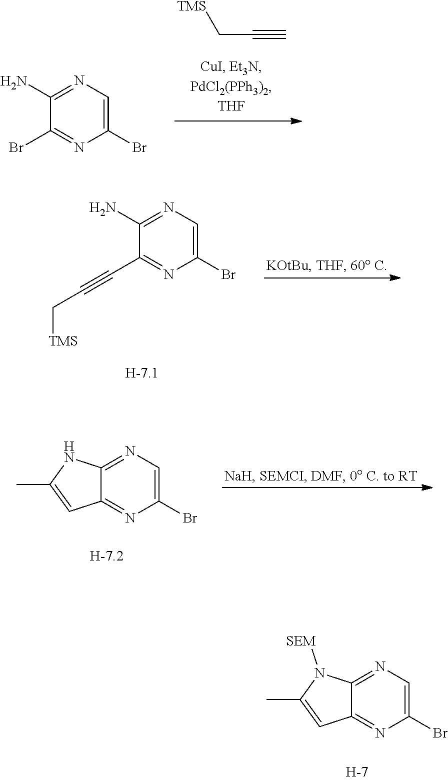

Synthesis of compound H-7.1. A mixture of 3,5-dibromopyrazin-2-amine (3 g, 11.6 mmol, 1.0 equiv), triethylamine (1.43 g, 14.24 mmol, 1.2 equiv), copper iodide (0.113 g, 0.59 mmol, 0.05 equiv) and 1,1-bis(triphenylphosphine)palladium(II) chloride (0.417 g, 0.59 mmol, 0.05 equiv) in THF (40 mL) was degassed by bubbling through a stream of argon for 10 min. Trimethyl(prop-2-yn-1-yl)silane (1.6 g, 14.24 mmol, 1.2 equiv) was added and the reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford H-7.1 (3.0 g, 89%). MS(ES): m/z 284.16 and 286.16 [M+1]⁺.

Synthesis of compound H-7.2. To a solution of H-7.1 (3 g, 10.55 mmol, 1.0 equiv) in THF (40 mL) was added 1 M potassium tert-butoxide in THF (21.12 mL, 21.12 mmol, 2.0 equiv). The reaction mixture was stirred at 60° C. for 2 h. It was diluted with ethyl acetate, washed with water, and then brine. The organic layer was dried over anhydrous sodium sulfate, filtered, and concentrated under reduced pressure to afford H-7.2 (1.1 g, 49%). It was used in the next step without purification. MS(ES): m/z 212.13 and 214.13 [M+1]⁺.

Synthesis of compound H-7. Compound H-7 was prepared from H-7.2 following the procedure described in the synthesis of H-1. The product was purified by silica gel flash column chromatography on silica gel (CombiFlash®, 10% ethyl acetate in hexane). MS(ES): m/z 342.07 and 344.07 [M+1]⁺.

### Preparation of Intermediate H-8: 6-bromo-3-((2-(trimethylsilyl)ethoxy)methyl)-3H-imidazo[4,5-b]pyridine

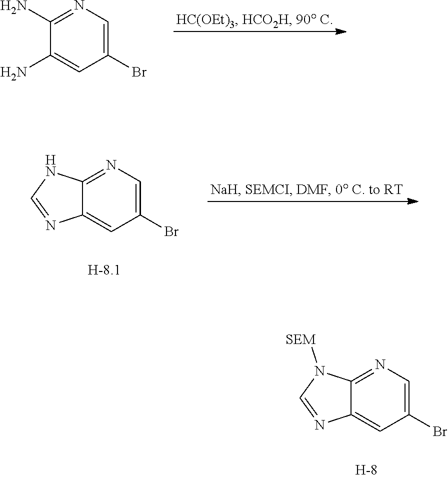 [SMILES: Nc1cc(Br)cnc1N ; Brc1cnc2[nH]cnc2c1 ; C[Si](C)(C)CCOCn1cnc2cc(Br)cnc21]

Synthesis of compound H-8.1. To a solution of 5-bromopyridine-2,3-diamine (3.0 g, 15.95 mmol, 1.0 equiv) in formic acid (1.1 mL) was added triethoxymethane (48 mL). The reaction mixture was stirred at 90° C. for 4 h. It was concentrated under reduced pressure. The residue was purified by flash column chromatography on silica gel (CombiFlash®, 15% ethyl acetate in hexane) to afford H-8.1 (2.74 g, 87%). MS(ES): m/z 198.03 and 200.02 [M+H]⁺.

Synthesis of compound H-8. Compound H-8 was prepared from H-8.1 following the procedure described in the synthesis of H-1. The product was purified by flash column chromatography on silica gel (CombiFlash®, 6.0% ethyl acetate hexane). MS(ES): m/z 328.1 and 330.1 [M+H]⁺.

### Preparation of Intermediate H-9: 2-bromo-7-fluoro-5-((2-(trimethylsilyl)ethoxy)methyl)-5H-pyrrolo[2,3-b]pyrazine

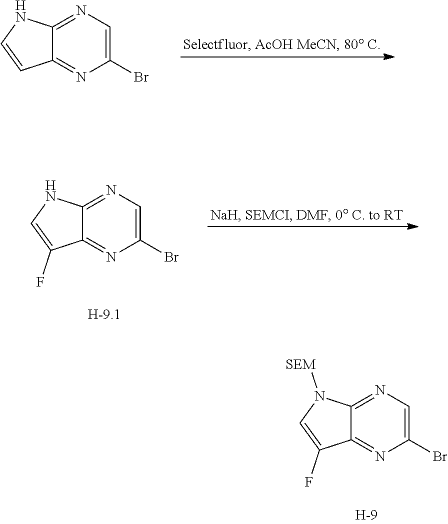 [SMILES: Brc1cnc2[nH]ccc2n1 ; Fc1c[nH]c2ncc(Br)nc12 ; C[Si](C)(C)CCOCn1cc(F)c2nc(Br)cnc21]

Synthesis of compound H-9.1 To a solution of 2-bromo-5H-pyrrolo[2,3-b]pyrazine (3.5 g, 17.67 mmol, 1.0 equiv) in acetonitrile (40 mL) and acetic acid (15 mL) was added dropwise Selectfluor (6.2 g, 17.67 mmol, 1.0 equiv) at room temperature. The reaction mixture was stirred at rt for 2 h and at 80° C. for 8 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate hexane) to afford H-9.1 (1.0 g, 26%). MS(ES): m/z [M+H]⁺.

Synthesis of compound H-9. Compound H-9 was prepared from H-9.1 following the procedure described in the synthesis of H-1. The product was used without purification. MS(ES): m/z [M+H]⁺.

### Preparation of Intermediate H-10: 2-bromo-7-cyclopropyl-5-((2-(trimethylsilyl)ethoxy)methyl)-5H-pyrrolo[2,3-b]pyrazine

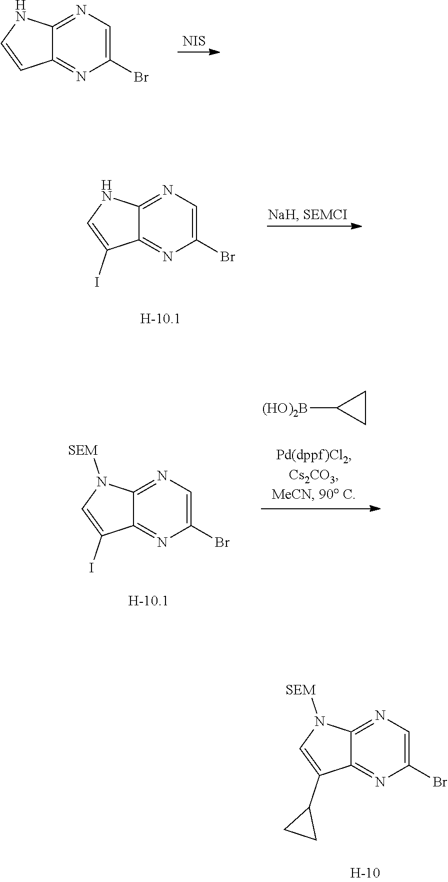 [SMILES: Brc1cnc2[nH]ccc2n1 ; Brc1cnc2[nH]cc(I)c2n1 ; OB(O)C1CC1 ; C[Si](C)(C)CCOCn1cc(I)c2nc(Br)cnc21 ; C[Si](C)(C)CCOCn1cc(C2CC2)c2nc(Br)cnc21]

Synthesis of compound H-10.1. To a solution of 2-bromo-5H-pyrrolo[2,3-b]pyrazine (3.0 g, 15.15 mmol, 1.0 equiv) in DMF (40 mL) was added N-iodosuccinimide (4.43 g, 19.69 mmol, 1.5 equiv) in small portions at 0° C. The reaction mixture was stirred at 25° C. for 16 h. It was transferred into ice-water. The precipitations were collected by filtration, washed with cold water, and dried under vacuum to afford H-10.1 (3.0 g, 62%). MS(ES): m/z 323.9 and 326.0 [M+H]⁺.

Synthesis of compound H-10.2. To a solution of H-10.1 (3.0 g, 9.25 mmol, 1.0 equiv) in DMF (30 mL) cooled to 0° C. was added sodium hydride (60 wt % in mineral oil, 0.399 g, 27.75 mmol, 3.0 equiv). The mixture was stirred for 30 min. 2-(Trimethylsilyl) ethoxymethylchloride (1.6 mL, 13.87 mmol, 1.5 equiv) was added and stirred at room temperature for 1 h. It was transferred into ice-water. The solids was collected by filtration and dried under vacuum to afford H-10.2 (2.8 g, 67%). MS(ES): m/z 454.06 and 456.04 [M+H]⁺.

Synthesis of compound H-10. A mixture of H-10.2 (2.8 g, 6.18 mmol, 1.0 equiv), cyclopropylboronic acid (2.6 g, 30.90 mmol, 5.0 equiv) and cesium carbonate (6.0 g, 18.54 mmol, 3.0 equiv) in acetonitrile (30 mL) was degassed by bubbling through a stream of argon for 10 min. [1,1′-Bis(diphenylphosphino)ferrocene]palladium(II) dichloride (0.451 g, 0.618 mmol, 0.1 equiv) was added and degassed for 5 min. The reaction mixture was stirred at 90° C. for 2 h. It was cooled to room temperature, transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford H-10 (0.7 g, 58.33%). MS(ES): m/z 368.14 and 370.16 [M+H]⁺.

### Preparation of Intermediate H-11: 5-bromo-N,N-dimethyl-1-((2-(trimethylsilyl)ethoxy)methyl)-1H-pyrazolo[3,4-b]pyridin-3-amine

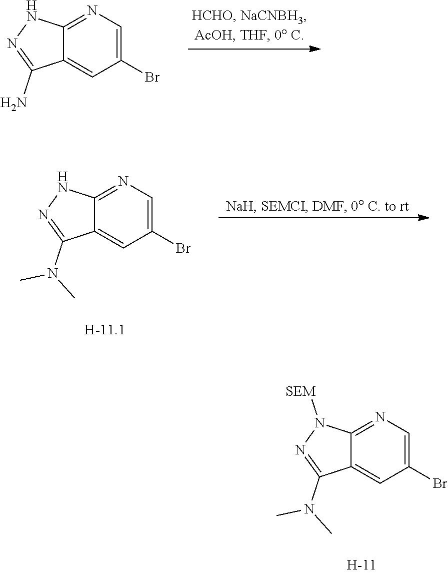 [SMILES: Nc1n[nH]c2ncc(Br)cc12 ; CN(C)c1n[nH]c2ncc(Br)cc12 ; CN(C)c1nn(COCC[Si](C)(C)C)c2ncc(Br)cc12]

Synthesis of compound H-11.1. To a solution of 5-bromo-1H-pyrazolo[3,4-b]pyridin-3-amine (0.8 g, 3.75 mmol, 1.0 equiv) in THF (20 mL) was added formaldehyde (3.3 g, 112.6 mmol, 30 equiv) and acetic acid (0.29 mL, 4.87 mmol, 1.3 equiv). The reaction mixture was stirred at 0° C. for 15 min and sodium cyanoborohydride (0.801 g, 12.75 mmol, 3.4 equiv) was added. It was stirred at room temperature 16 h, transferred into a saturated solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 4.0% methanol in DCM) to afford H-11.1 (0.5 g, 56%). MS(ES): m/z: 241.18 and 243.20 [M+H]⁺.

Synthesis of compound H-11. Compound H-11 was prepared from H-11.1 following the procedure described in the synthesis of H-1. The product was purified by flash column chromatography on silica gel (CombiFlash®, 3.1% methanol in DCM). MS(ES): m/z 371.29 and 373.29 [M+H]⁺.

### Preparation of Intermediate H-13: 5-bromo-3-methoxy-1-(4-methoxybenzyl)-1H-pyrazolo[3,4-b]pyridine

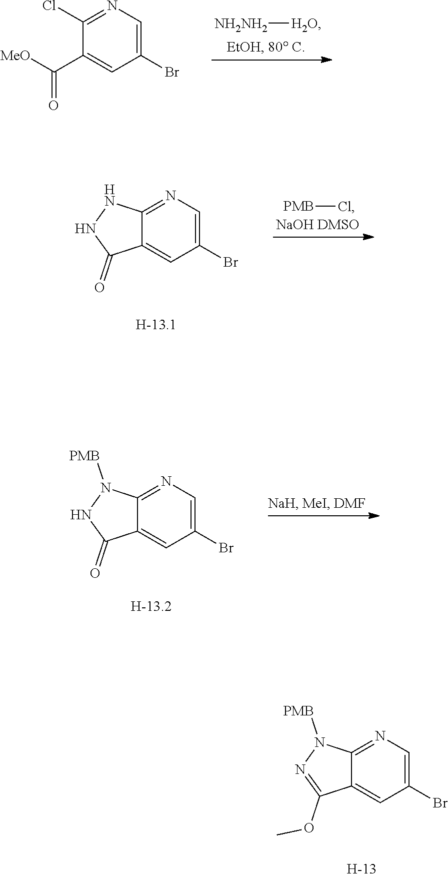

Synthesis of compound H-13.1. To a solution of methyl 5-bromo-2-chloronicotinate (5.0 g, 19.96 mmol, 1.0 equiv) in ethanol (80 mL) was added hydrazine hydrate (7.0 g, 139.73 mmol, 7.0 equiv) and the reaction mixture was stirred at 80° C. for 16 h. It was cooled to 10° C. The solids precipitated were collected by filtration, rinsed with ethanol and water, dried under vacuum to afford H-13.1 (2.10 g, 49%). MS(ES): m/z 214.02 and 216.01 [M+H]⁺.

Synthesis of compound H-13.2. To a solution of H-13.1 (2.10 g, 9.81 mmol, 1.0 equiv) in dimethyl sulfoxide (25.0 mL) was added sodium hydroxide (0.581 g, 14.72 mmol, 1.5 equiv) at 0° C., followed by the addition of 4-methoxybenzyl chloride (2.3 g, 14.72 mmol, 1.5 equiv). The reaction mixture was stirred at room temperature for 2 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (45% ethyl acetate in hexane) to afford H-13.2 (1.30 g, 40%). MS(ES): m/z 333.16 and 335.1 [M+H]⁺.

Synthesis of compound H-13. To a solution of H-13.2 (1.3 g, 3.89 mmol, 1 equiv) in DMF (12 mL) at 0° C. was added sodium hydride (60%, 0.186 g, 4.67 mmol, 1.20 equiv). The mixture was stirred at 0° C. for 10 min, followed by the addition of methyl iodide (0.662 g, 4.67 mmol, 1.20 equiv). It was stirred for 1.5 h, poured into ice-water, and extracted with ethyl acetate. The organic layer was washed with brine, dried over anhydrous sodium sulfate, filtered and concentrated. The residue was purified by flash column chromatography on silica gel (CombiFlash®, 40% ethyl acetate in hexane) to afford H-13 (0.78 g, 58%). MS(ES): m/z: 348.12 and 350.12 [M+H]⁺.

### Preparation of Intermediate H-15: 2-bromo-6-(methoxymethyl)-5-((2-(trimethylsilyl)ethoxy)methyl)-5H-pyrrolo[2,3-b]pyrazine

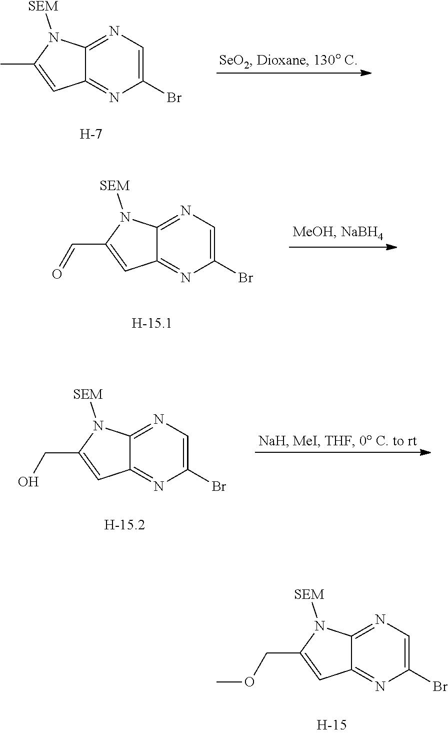 [SMILES: Cc1cc2nc(Br)cnc2n1COCC[Si](C)(C)C ; C[Si](C)(C)CCOCn1c(C=O)cc2nc(Br)cnc21 ; C[Si](C)(C)CCOCn1c(CO)cc2nc(Br)cnc21 ; COCc1cc2nc(Br)cnc2n1COCC[Si](C)(C)C]

Synthesis of compound H-15.1. To solution of H-7 (1.8 g, 5.26 mmol, 1.0 equiv) in 1,4-dioxane (20 mL) at 0° C. was added selenium dioxide (0.727 g, 55.55 mmol, 4.0 equiv) in portions. The reaction mixture was stirred at 130° C. for 16 h. It was filtered through a pad of Celite® and filtrate was extracted with ethyl acetate. The combined organic layers were washed with brine, dried over anhydrous sodium sulfate, The residue was purified by flash column chromatography on silica gel (CombiFlash®, 25% ethyl acetate in hexane to afford H-15.1 (1.2 g, 64%), MS(ES): m/z 357.1 and 359.1 [M+H]⁺.

Synthesis of compound H-15.2. To a solution of H-15.1 (1.2 g, 3.37 mmol, 1.0 equiv) in methanol (15 mL) was added sodium borohydride (0.192 g, 5.05 mmol, 1.5 equiv) in portions at 0° C. The reaction mixture was stirred at room temperature for 6 h. It was quenched with a saturated solution of ammonium chloride and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford H-15.2 (0.80 g, 66%). MS(ES): m/z 359.15 and 361.1 [M+H]⁺.

Synthesis of compound H-15. To a solution of H-15.2 (0.800 g, 2.23 mmol, 1.0 equiv) in THE (10 mL) at 0° C. was added sodium hydride (0.065 g, 3.35 mmol, 1.5 equiv) in portions and stirred for 15 min at 0° C. To the mixture was added methyl iodide (0.4 mL, 6.69 mmol, 3.0 equiv) and the reaction mixture was stirred at room temperature for 2 h. After completion of reaction, the mixture was quenched with saturated solution of ammonium chloride and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford H-14 (0.50 g, 60.24%). MS(ES): m/z 374.12 and 376.12 [M+H]⁺.

### Preparation of Intermediate H-16: 5-bromo-2-methyl-1-((2-(trimethylsilyl)ethoxy)methyl)-1,2-dihydro-3H-pyrazolo[3,4-b]pyridin-3-one

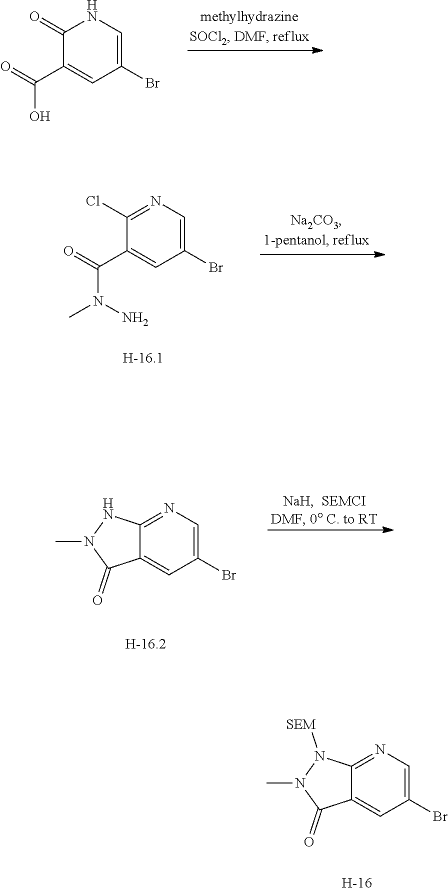 [SMILES: O=C(O)c1cc(Br)c[nH]c1=O ; CN(N)C(=O)c1cc(Br)cnc1Cl ; Cn1[nH]c2ncc(Br)cc2c1=O ; Cn1c(=O)c2cc(Br)cnc2n1COCC[Si](C)(C)C]

Synthesis of compound H-16.1. To 5-bromo-2-oxo-1,2-dihydropyridine-3-carboxylic acid (10 g, 46.29 mmol, 1.0 equiv) at 0° C. was added thionyl chloride (100 mL) and catalytic amount of DMF (0.5 mL). The reaction mixture was stirred at 80° C. for 16 h. It was concentrated and azeotroped with toluene and resulting acid chloride was dissolved in DCM (100 mL). It was cooled to 0° C. and a mixture of methyl hydrazine (2.4 mL, 46.29 mmol, 1.0 equiv), sodium hydroxide (7.4 g, 185 mmol, 4.0 equiv) and water (15 mL) was added. The reaction mixture was stirred at 80° C. for 30 min. It was diluted with water and extracted with DCM. The combined organic layers were washed with brine, dried over anhydrous sodium sulfate, filtered, and concentrated under reduced pressure. The residue was purified by flash column chromatography on silica gel (CombiFlash®, 5.0% methanol in DCM) to afford H-16.1 (3.0 g, 25%). MS(ES): m/z 264.3 and 266.3 [M+H]⁺.

Synthesis of compound H-16.2. To stirred solution of compound H-16.1 (3.0 g, 11.36 mmol, 1 equiv) in 1-pentanol (30 mL) was added sodium carbonate (1.2 g, 11.36 mmol, 1.0 equiv). The reaction mixture was stirred at 140° C. for 48 h under argon. It was cooled to room temperature and acidified with acetic acid. Most solvents were removed under reduced pressure and the residue was purified by flash chromatography (ethyl acetate-methanol 9:1) to afford H-16.2 (1.0 g, 38%) MS(ES): m/z 288.01 and 300.01 [M+H]⁺.

Synthesis of compound H-16. Compound H-16 was prepared from H-16.2 following the procedure described in the synthesis of H-1. The product was purified by flash column chromatography on silica gel (CombiFlash®, 3.0% methanol in DCM). MS(ES): m/z 358.1 and 360.1[M+H]⁺.

### Preparation of Intermediate H-17: N-(3-bromopyrazolo[1,5-a]pyrazin-6-yl)-N-methylcyclopropanecarboxamide

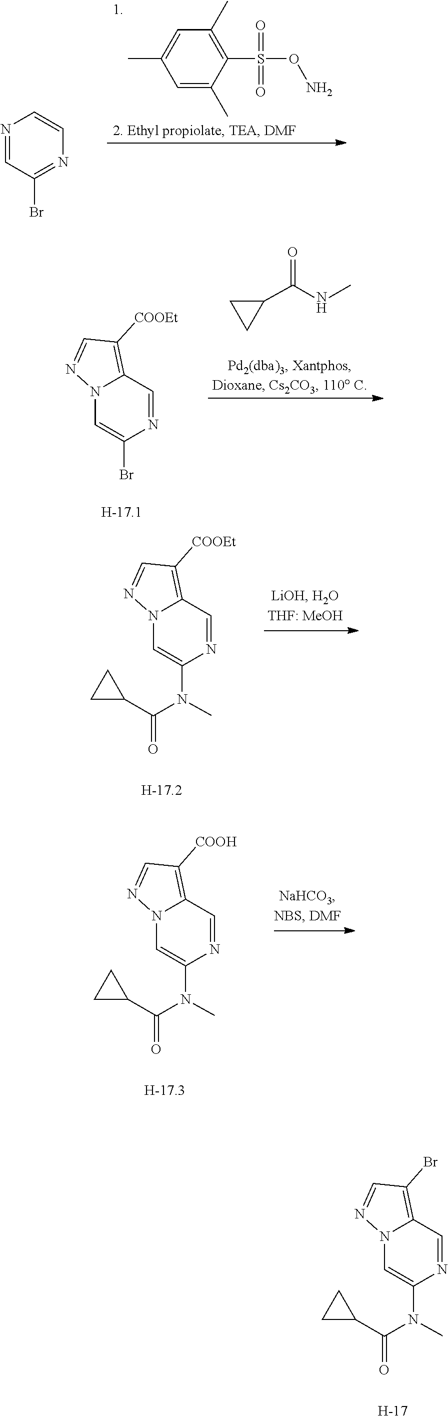

Synthesis of compound H-17.1. To a solution of 2-bromopyrazine (432.0 g, 2720 mmol, 1.0 equiv) in DCM (5180 mL), was added O-(mesitylsulfonyl)hydroxylamine (701.9 g, 3260 mmol, 1.2 equiv) at 0° C. The reaction mixture was stirred at room temperature for 1 h. It was transferred into diethyl ether. The solids precipitated were collected by filtration and dried under vacuum. To a solution of the solids in DMF (2160 mL) was added trimethylamine (372.2 g, 3685 mmol, 2.0 equiv) followed by ethyl propiolate (361.16 g, 3685 mmol, 2.0 equiv) at 0° C. The reaction mixture was stirred at room temperature for 48 h. It was transferred into brine, extracted with ethyl acetate. The combined organic layers were washed with water solution, dried over anhydrous sodium sulfate, filtered, and concentrated under reduced pressure. The residue was purified by flash column chromatography on silica gel (4% ethyl acetate in hexane) to afford H-17.1 (20 g, 2.7%). %). MS (ES): m/z 270.1 and 272.1 [M+H]⁺.

Synthesis of compound H-17.2. A mixture of H-17.1 (1.5 g, 4.95 mmol, 1.0 equiv), N-methylcyclopropanecarboxamide (1.3 g, 14.85 mmol, 3.0 equiv) and cesium carbonate (4.8 g, 14.85 mmol, 3.0 equiv) in 1,4-dioxane (10 mL) was degassed by bubbling through a stream of argon for 10 min. 4,5-Bis(diphenylphosphino)-9,9-dimethylxanthene (0.572 g, 0.99 mmol, 0.2 equiv) and tris(dibenzylidene acetone)dipalladium (O) (0.452 g, 0.495 mmol, 0.1 equiv) were added and degassed for 5 min. The reaction mixture was stirred at 110° C. for 1 h. It was cooled to room temperature, transferred into water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.1% methanol in DCM) to afford J-17.2 (0.75 g, 54%). MS(ES): m/z: 289.12 [M+H]⁺.

Synthesis of compound H-17.3. To a solution of H-17.2 (0.75 g, 2.59 mmol, 1.0 equiv) in methanol (3.0 mL), THF (3.0 mL) and water (2.0 mL) was added lithium hydroxide (0.163 g, 3.89 mmol, 1.5 equiv) in portions. The reaction mixture was stirred at room temperature for 1 h. It was extracted with ethyl acetate. The aqueous layer was separated, adjusted to pH 2 using 1.5 N hydrochloric acid, and extracted with ethyl acetate. The organic layer was washed with brine, dried over anhydrous sodium sulfate, filtered, and concentrated under reduced pressure to afford H-17.3 (0.50 g, 74%). MS(ES): m/z 261.09 [M+H]⁺.

Synthesis of compound H-17. To a solution of H-17.3 (0.50 g, 1.92 mmol, 1.0 equiv) in DMF (3.0 mL) were added sodium bicarbonate (0.482 g, 5.74 mmol, 3.0 equiv) and N-bromosuccinimide (0.375 g, 2.11 mmol, 1.1 equiv), and the mixture stirred for 6 h at room temperature. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford H-17 (0.28 g, 49%). MS(ES): m/z 295.01 [M+H]⁺.

### Preparation of Intermediate H-18: 5-bromo-3-methoxy-1-tosyl-1H-pyrrolo[2,3-b]pyridine

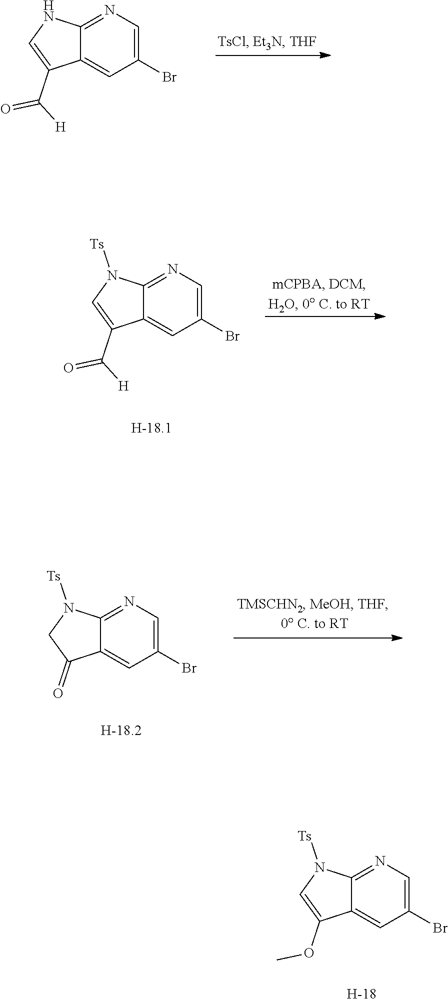 [SMILES: O=Cc1c[nH]c2ncc(Br)cc12 ; Cc1ccc(S(=O)(=O)n2cc(C=O)c3cc(Br)cnc32)cc1 ; Cc1ccc(S(=O)(=O)N2CC(=O)c3cc(Br)cnc32)cc1 ; COc1cn(S(=O)(=O)c2ccc(C)cc2)c2ncc(Br)cc12]

Synthesis of compound H-18.1. To solution of 5-bromo-1H-pyrrolo[2,3-b]pyridine-3-carbaldehyde (5.0 g, 22.22 mmol, 1.0 equiv) in THF (100 mL) was added triethylamine (3.5 mL, 24.44 mmol, 1.1 equiv) and p-toluene sulfonyl chloride (4.2 g, 22.22 mmol, 1.0 equiv). The mixture was stirred at room temperature for 12 h. It was poured into water and extracted with ethyl acetate. The organic layer was washed with water, brine, dried over anhydrous sodium sulfate, and concentrated under reduced pressure. The residue was added DCM-diethyl ether and the solids were collected by filtration, rinsed with hexane and dried under vacuum to afford H-18.1 (3.2 g, 50%). MS(ES): m/z 379.1 and 380.1 [M+H]⁺.

Synthesis of compound H-18.2. To a solution of H-18.1 (3.2 g, 8.42 mmol, 1.0 equiv) in DCM (50 mL) at 0° C. was added 3-chloroperbenzoic acid (3.1 g, 18.52 mmol, 2.2 equiv) in portions over a period of 30 min. The mixture was stirred at room temperature for 2 h and was added a 10% aqueous sodium sulfite solution and a saturated aqueous sodium bicarbonate solution and stirred for 6 h. It was diluted with DCM and the solids were removed by filtration. The filtrate was extracted with DCM. The organic layer was separated, dried over anhydrous sodium sulfate, filtered, and concentrated under reduced pressure. The residue was purified by flash column chromatography on silica gel (35% ethyl acetate in hexane) to afford H-18.2 (0.395 g, 13%). MS(ES): m/z 367.1 and 369.1 [M+H]⁺.

Synthesis of compound H-18. To a solution of H-18.2 (0.395 g, 1.07 mmol, 1.0 equiv) in methanol (15 mL) and THE (15 mL) at 0° C. was added (trimethylsilyl)diazomethane (2.1 mL, 4.28 mmol, 4.0 equiv) dropwise. The reaction mixture was stirred at room temperature for 4 h. An additional portion of trimethylsilyl)diazomethane (2.1 mL, 4.28 mmol, 4.0 equiv) was added dropwise and stirred for 12 h. It was quenched with acetic acid (0.006 mL, 0.107 mmol, 0.1 equiv) and the mixture was concentrated under reduced pressure. The residue was purified by flash column chromatography on silica gel (30% ethyl acetate-hexane) to afford H-18 (0.205 g, 50%). MS(ES): m/z 379.1 and 381.03 [M+H]⁺.

### Preparation of Intermediate H-19: 5-bromo-N-methyl-1H-pyrazolo[3,4-b]pyridin-3-amine

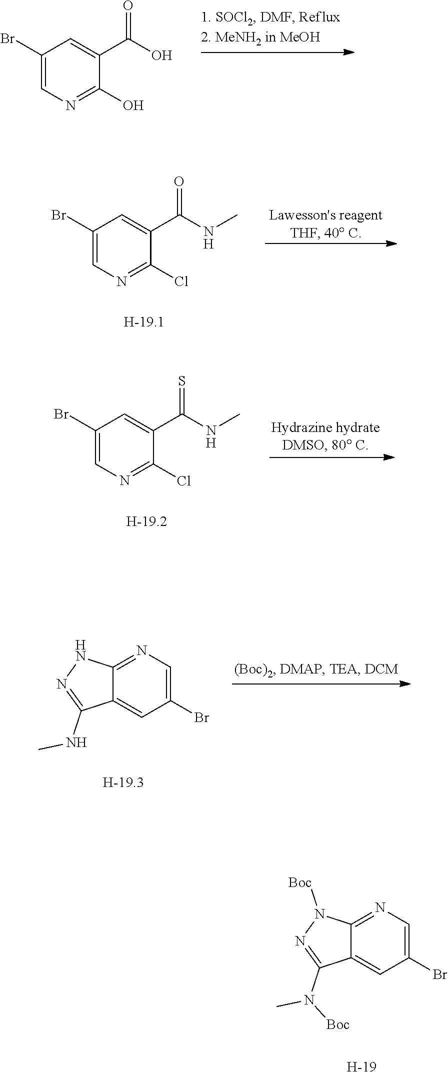

Synthesis of compound H-19.1. To compound 5-bromo-2-hydroxynicotinic acid (50 g, 229.35 mmol, 1.0 equiv) at 0° C. was added thionyl chloride (500 mL) and catalytic amount of DMF (4.0 mL). The reaction mixture was stirred at 80° C. for 16 h. It was concentrated and azeotroped with toluene. The resulting acid chloride was dissolved in DCM (500 mL) and cooled to 0° C. and added a solution of methylamine in methanol (8.02 g, 229.35 mmol, 1.0 equiv), sodium hydroxide (37.0 g, 917.41 mmol, 4.0 equiv) and water (150 mL). The reaction mixture was stirred at 80° C. for 30 min. It was diluted with water and extracted with DCM. The combined organic layers were washed with brine, dried over anhydrous sodium sulfate, filtered, and concentrated under reduced pressure. The residue was purified by flash column chromatography on silica gel (CombiFlash®, 5.0% methanol in DCM) to afford H-19.1 (32.0 g, 56%). MS(ES): m/z 249.6 and 251.6 [M+H]⁺.

Synthesis of compound H-19.2. To a solution of H-19.1 (32.0 g, 128.26 mmol, 1.0 equiv) in THF (400.0 m) was added Lawesson's reagent (207 g, 256.52 mmol, 2.0 equiv) and stirred at 40° C. for 24 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 29% ethyl acetate in hexane) to afford H-19.2 (16.3 g, 48%). MS(ES): m/z 265.2 and 267.2 [M+H]⁺.

Synthesis of compound H-19.3. To a solution of H-19.2 (16.3 g, 61.38 mmol, 1.0 equiv) in dimethyl sulfoxide (200 mL) was added hydrazine hydrate (9.2 g 184 mmol, 3.0 equiv) and stirred at 80° C. for 24 h. It transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 50% ethyl acetate in hexane) to afford H-19.3 (8.6 g, 62%). MS(ES): m/z 227.1 and 229.1 [M+H]⁺.

Synthesis of compound H-19. To a solution of H-19.3 (0.68 g, 2.99 mmol, 1.0 equiv, triethylamine (0.90 g 8.98 mmol, 3.0 equiv) and 4-dimethylaminopyridine (0.72 g 5.98 mmol, 3.0 equiv), in DCM (8.0 mL) was added di-tert-butyl dicarbonate (1.95 g 8.98 mmol, 3.0 equiv) and stirred at rt for 24 h. It transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford H-19 (0.32 g, 25%). MS(ES): m/z 427.2 and 429.2 [M+H]⁺.

### Preparation of Intermediate H-20: 6-bromo-1,3-bis(4-methoxybenzyl)-1,3-dihydro-2H-imidazo[4,5-b]pyridin-2-one

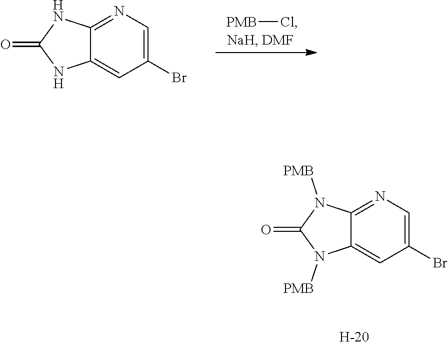

Synthesis of compound H-20. To a solution of 6-bromo-1,3-dihydro-2H-imidazo[4,5-b]pyridin-2-one (1.0 g, 4.67 mmol, 1.0 equiv) in DMF (10 mL) at 0° C. was added in portions sodium hydride (60% in mineral oil) (0.441 g, 10.28 mmol, 2.2 equiv) and stirred for 15 min. 4-Methoxybenzyl chloride (1.6 g, 10.28 mmol, 2.2 equiv) was added and stirred at room temperature for 2 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford H-20 (0.80 g, 38%). MS(ES): m/z 454.3 and 456.11 [M+H]⁺.

### Preparation of Intermediate H-21: 5-bromo-1-(4-methoxybenzyl)-3-(oxetan-3-yloxy)-1H-pyrazolo[3,4-b]pyridine

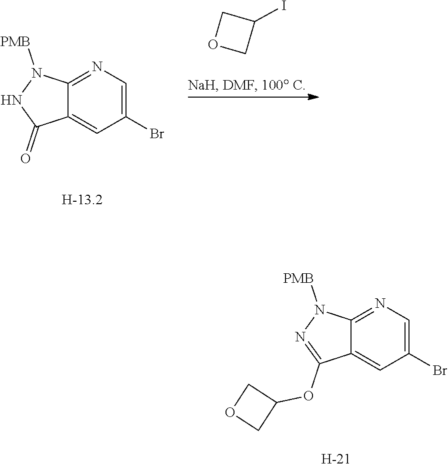

Synthesis of compound H-21. To a solution of H-13.2 (1.0 g, 2.99 mmol, 1.0 equiv) in DMF (10 mL) at 0° C. was added sodium hydride (0.129 g, 8.97 mmol, 3.0 equiv). The mixture was stirred for 30 min and 3-iodooxetane (0.658 g, 3.58 mmol, 1.2 equiv) was added. The reaction mixture was stirred at 100° C. for 2 h. It was transferred into ice-water and extracted with ethyl acetate. The combined organic layers were washed with brine, dried over anhydrous sodium sulfate, filtered, and concentrated under reduced pressure to afford H-21 (0.659 g, 56%). MS(ES): m/z 390.03 and 392.01 [M+H]⁺.

### Preparation of Intermediate H-22: (R)-3-((1,4-dioxan-2-yl)methoxy)-5-bromo-1-(4-methoxybenzyl)-1H-pyrazolo[3,4-b]pyridine

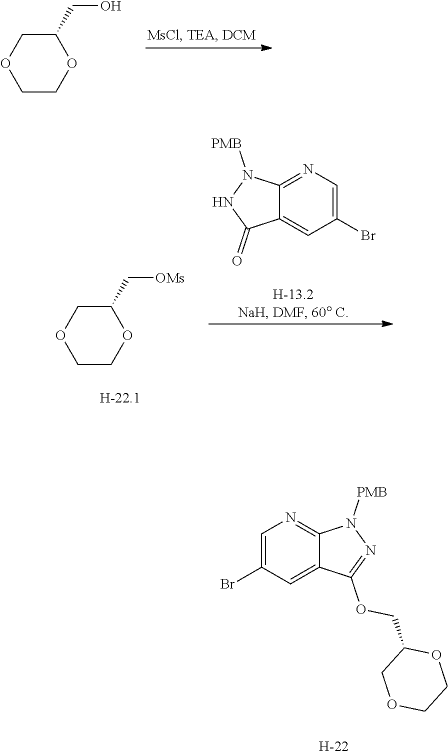

Synthesis of compound H-22.1. To a solution of (S)-(1,4-dioxan-2-yl)methanol (0.60 g, 5.08 mmol, 1.0 equiv) and triethylamine (1.4 mL, 10.16 mmol, 2.0 equiv) in DCM (10 mL) at 0° C. was added methanesulfonyl chloride (0.5 mL, 6.60 mmol, 1.3 equiv) and the reaction mixture was stirred at room temperature for 6 h. It was transferred into ice-water and extracted with ethyl acetate. The combined organic layers were washed with brine, dried over anhydrous sodium sulfate, filtered, and concentrated under reduced pressure to afford H-22.1 (0.7 g, 70%). MS(ES): m/z 197.04 [M+H]⁺.

Synthesis of compound H-22. To a solution of H-13.2 (0.70 g, 2.08 mmol, 1.0 equiv) in DMF (3.0 mL) at 0° C. was added sodium hydride (0.089 g, 6.24 mmol, 3.0 equiv). The mixture was stirred for 30 min and H-22.1 (0.491 g, 2.50 mmol, 1.2 equiv) was added. The reaction mixture was stirred at 60° C. for 2 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.0% methanol in DCM) to afford H-22 (0.50 g, 55%). MS(ES): m/z 434.1 and 436.04 [M+H]⁺.

### Preparation of Intermediate H-22: 5-bromo-1-(4-methoxybenzyl)-3-(2-methoxyethoxy)-1H-pyrazolo[3,4-b]pyridine

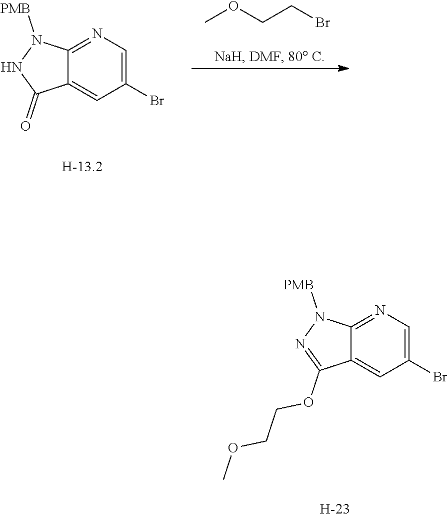

Synthesis of compound H-23. To a solution of H-13.2 (1.0 g, 2.99 mmol, 1.0 equiv) in DMF (10.0 mL) was added sodium hydride (60% in mineral oil) (0.141 g, 3.59 mmol, 1.2 equiv) in portions at 0° C. and stirred for 15 min. 1-Bromo-2-methoxyethane (0.49 g, 3.59 mmol, 1.2 equiv) was added and stirred at room temperature for 2 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford H-23 (0.620 g, 53%). MS(ES): m/z 392.16 and 394.17 [M+H]⁺.

### Preparation of Intermediate H-24: 5-bromo-1-(4-methoxybenzyl)-3-((tetrahydro-2H-pyran-4-yl)oxy)-1H-pyrazolo[3,4-b]pyridine

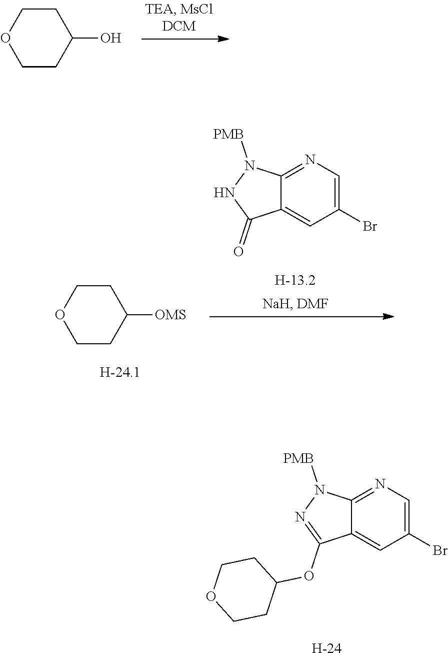

Synthesis of compound H-24.1. To a solution of tetrahydro-2H-pyran-4-ol (2.0 g, 19.60 mmol, 1.0 equiv), trimethylamine (8.4 mL, 58.8 mmol, 3.0 equiv) in DCM (20 mL) was added methanesulfonyl chloride (2.26 mL, 29.4 mmol, 1.5 equiv) at 0° C. The mixture was stirred at room temperature for 30 min. It was quenched with water (30 mL) and extracted with DCM (50 mL). The combined organic layers were washed with brine, dried over anhydrous sodium sulfate, filtered, and concentrated under reduced pressure to afford H-24.1 (2.2 g, 100%). MS(ES): m/z 181.05 [M+H]⁺.

Synthesis of compound H-24. To a solution of H-13.2 (1.0 g, 3.00 mmol, 1.0 equiv) in DMF (10 mL) at 0° C. was added sodium hydride (0.129 g, 9.0 mmol, 3.0 equiv). The mixture was stirred for 30 min. To the mixture was added H-24.1 (0.81 g, 4.50 mmol, 1.5 equiv) and the reaction mixture was stirred at room temperature for 2 h. It was transferred into ice-water and extracted with ethyl acetate. The combined organic layers were washed with brine, dried over anhydrous sodium sulfate, filtered, and concentrated under reduced pressure to afford H-24 (0.48 g, 38%). MS(ES): m/z 418.20 And 420.20 [M+H]⁺.

### Preparation of Intermediate H-25: 5-bromo-2-(tetrahydro-2H-pyran-4-yl)-1-((2-(trimethylsilyl)ethoxy)methyl)-1,2-dihydro-3H-pyrazolo[3,4-b]pyridin-3-one

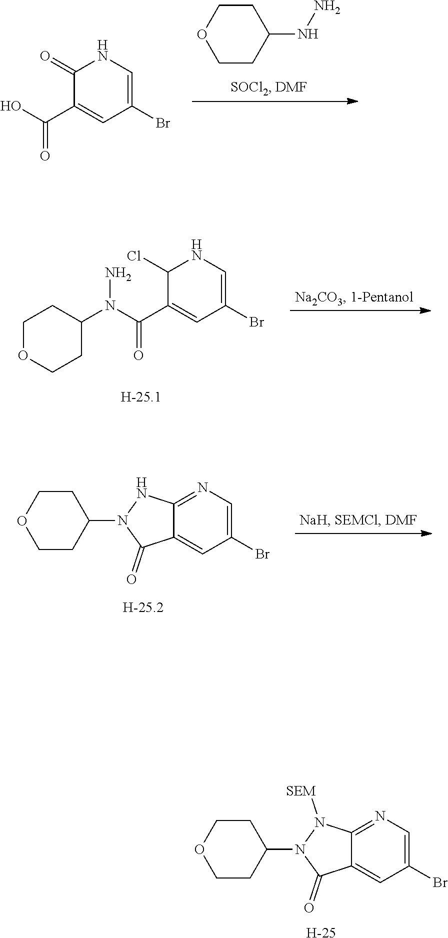

Synthesis of compound H-25. Compound H-25 was prepared following the procedures described in the synthesis of H-16. MS(ES): m/z 428.2 and 430.2 [M+H]⁺.

### Preparation of Intermediate H-26: trans-2-(3-(benzyloxy)cyclobutyl)-5-bromo-1-((2-(trimethylsilyl)ethoxy)methyl)-1,2-dihydro-3H-pyrazolo[3,4-b]pyridin-3-one

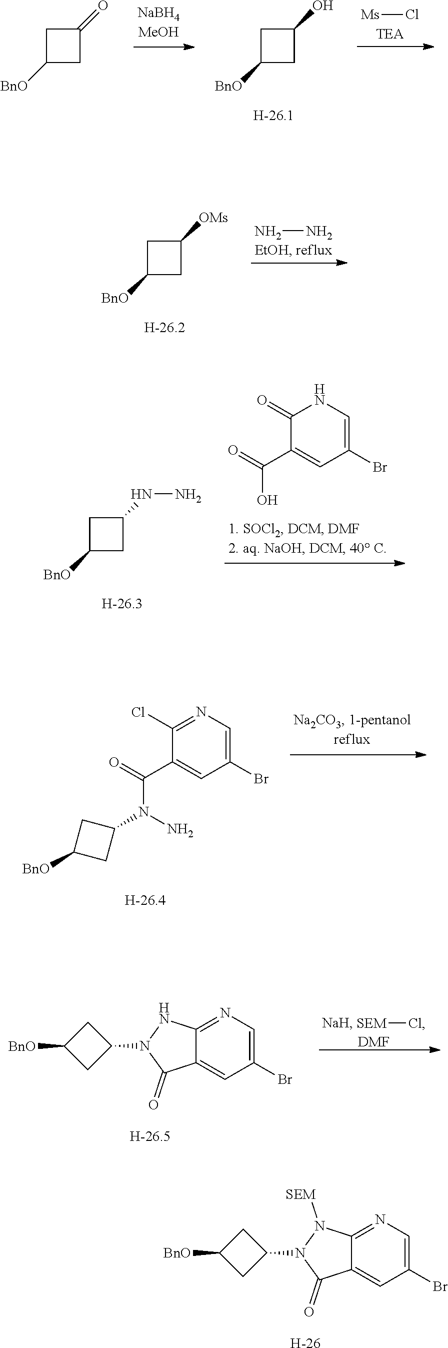

Synthesis of compound H-26.1. A solution of 3-(benzyloxy)cyclobutan-1-one (5.0 g, 28.40 mmol, 1.0 equiv) in methanol (50 mL) at 0° C. and sodium borohydride (1.29 g, 34.05 mmol, 1.2 equiv) was added to it portions. The resulting reaction mixture was stirred at room temperature for 0.5 h. It was quenched with water (100 mL) and extracted with DCM. The combined organic layers were dried over anhydrous sodium sulfate, filtered, and concentrated under reduced pressure to afford the material. The residue was purified by flash column chromatography on silica gel (CombiFlash®, 28% ethyl acetate in hexane) to afford H-26.1 (5.0 g, 99%). MS(ES): m/z 179.23 [M+H]⁺.

Synthesis of compound H-26.2. To a solution of H-26.1 (5.0 g, 29.175 mmol, 1.0 equiv) and triethylamine (16.2 mL, 116.7 mmol, 4.0 equiv) in DCM (52 mL) was added methanesulfonyl chloride (4.5 mL, 58.35 mmol, 2.0 equiv) at 0° C. The reaction mixture was stirred at room temperature for 0.5 h. The reaction mixture was quenched with water (100 mL) and extracted with DCM. The combined organic layers were washed with saturated sodium chloride solution (30 mL), dried over anhydrous sodium sulphate, concentrated under reduced pressure to afford the material. This was then purified by flash column chromatography on silica gel (CombiFlash®, 35% ethyl acetate in hexane) to afford H-26.2 (5.0 g, 63%). MS(ES): m/z 257.32 [M+H]⁺.

Synthesis of compound H-26.3. To a solution of H-26.2 (5.0 g, 19.53 mmol, 1.0 equiv) in ethanol (50 mL) was added hydrazine (1.25 g, 30.26 mmol, 2.0 equiv) at room temperature. The reaction mixture was stirred at 95° C. for 6 h. It was concentrated under reduced pressure to afford H-26.3 (3.71 g, 99%). MS(ES) m/z 193.26 [M+H]⁺.

Synthesis of compound H-26.4. To a solution of 5-bromo-2-oxo-1,2-dihydropyridine-3-carboxylic acid (4.2 g, 19.27 mmol, 1.0 equiv) in DCM (40 mL) and DMF (1 mL) at 0° C. was added thionyl chloride (4 mL). The mixture was stirred at 40° C. for 1 h. It was concentrated under reduced pressure. The resulting acyl chloride was dissolved in DCM (58 mL) and was added 1 M sodium hydroxide solution (29 mL) and H-26.3 (3.7 g, 19.27 mmol, 1.0 equiv) at 0° C. The mixture was stirred at 50° C. for 15 min. It was quenched with water (200 mL) and extracted with DCM. The combined organic layers were dried over anhydrous sodium sulfate, filtered, and concentrated under reduced pressure. The residue was purified by flash column chromatography on silica gel (CombiFlash®, 14% ethyl acetate in hexane) to afford H-26.4 (1.8 g, 23%). MS(ES): m/z 411.70 [M+H]⁺.

Synthesis of compound H-26.5. A mixture of H-26.45 (1.8 g, 4.39 mmol, 1 equiv) and sodium carbonate (1.39 g, 13.17 mmol, 3 equiv) in 1-pentanol (18 mL) was stirred at 140° C. for 2 days. It was concentrated under reduced pressure to afford the material. The residue was purified by flash column chromatography on silica gel (CombiFlash®, 52% ethyl acetate in hexane) to afford H-26.5 (1.6 g, 98%). MS(ES): m/z 374.2 and 376.2 [M+H]⁺.

Synthesis of compound H-26. Compound H-26 was prepared from H-26.5 following the procedure described in the synthesis of H-1. The product was purified by flash column chromatography on silica gel (CombiFlash®, 30% ethyl acetate in hexane). MS(ES): m/z 504.3 and 505.2 [M+H]⁺.

### Preparation of Intermediate H-27: tert-butyl 4-(5-bromo-1-((2-(trimethylsilyl)ethoxy)methyl)-1H-pyrazolo[3,4-b]pyridin-3-yl)-3, 6-dihydropyridine-1(2H)-carboxylate

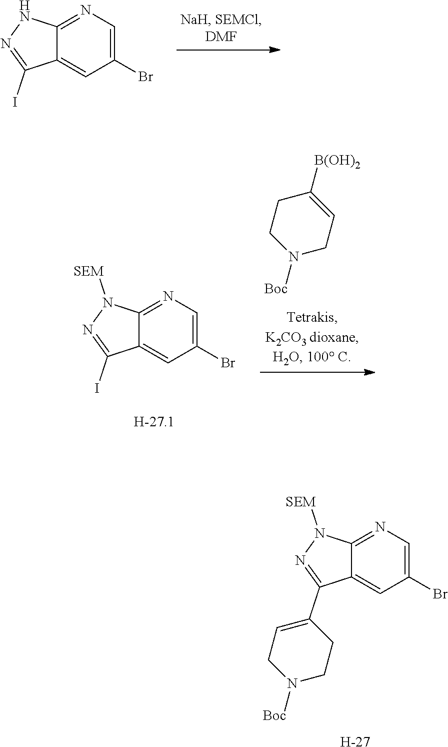

Synthesis of compound H-27.1. Compound H-27.1 was prepared from 5-bromo-3-iodo-1H-pyrazolo[3,4-b]pyridine following the procedure described in the synthesis of H-1. The product was purified by flash column chromatography on silica gel (CombiFlash®, 30% ethyl acetate in hexane). MS(ES): m/z 453.1 and 455.0 [M+H]⁺.

Synthesis of compound H-27. A mixture of H-27.1 (2.0 g, 4.4 mmol, 1.0 equiv), (1-(tert-butoxycarbonyl)-1,2,3,6-tetrahydropyridin-4-yl)boronic acid (1.2 g, 17.05 mmol, 1.5 equiv) and potassium carbonate (1.52 g, 11.02 mmol, 2.50 equiv) in 1,4-dioxane (15.0 mL) and water (5 mL) was degassed by bubbling through a stream of argon for 20 min. Tetrakis(triphenylphosphine)palladium(O) (0.073 g, 0.44 mmol, 0.1 equiv) was added and degassed for 5 min. The reaction mixture was stirred at 100° C. for 2 h. It was filtered through a pad of Celite® and rinsed with ethyl acetate. The filtrate added water and extracted with ethyl acetate. The organic layer was dried over anhydrous sodium sulfate, filtered, and concentrated under reduced pressure. The residue was purified by flash column chromatography on silica gel (CombiFlash®, 30% ethyl acetate in hexane) to afford H-27 (1.68 g, 75%). MS(ES): m/z 509.2 and 511.2 [M+H]⁺.

### Preparation of Intermediate H-28: 4-(2-bromo-5-((2-(trimethylsilyl)ethoxy)methyl)-5H-pyrrolo[2,3-b]pyrazin-7-yl)morpholine

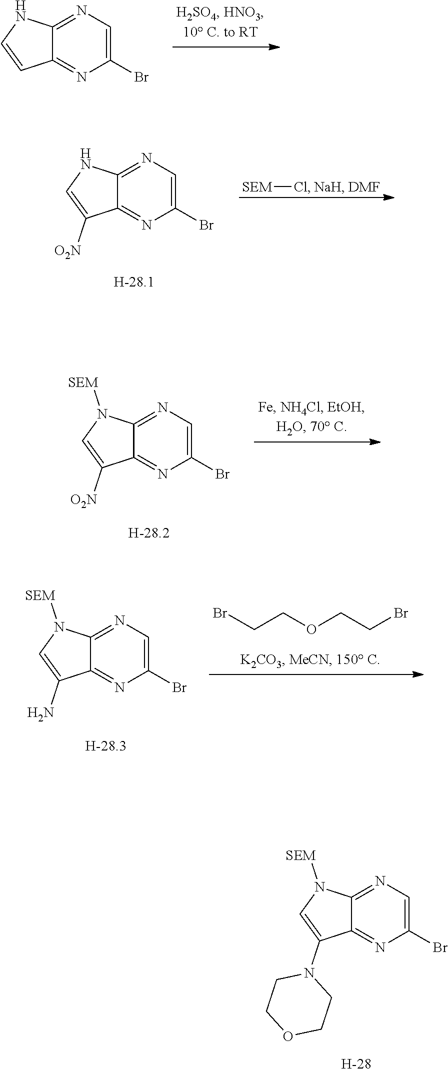

Synthesis of compound H-28.1. Compound 2-bromo-5H-pyrrolo[2,3-b]pyrazine (2.0 g, 10.101 mmol, 1.0 equiv) was slowly added to concentrated sulfuric acid (14 mL) at 0° C. followed by the addition of fuming nitric acid (0.8 mL). The mixture was stirred at room temperature for 16 h. The reaction mixture was poured into ice-water. A precipitate was formed and allowed to age for 30 min. The precipitate was collected by filtration and washed with water (200 mL) and dried under vacuum to afford H-28.1 (1.9 g, 78%). MS(ES): m/z 243.07 and 245.07 [M+H]⁺.

Synthesis of compound H-28.2. To a solution of H-28.1 (1.6 g, 6.58 mmol, 1.0 equiv) in DMF (16 mL) at 0° C. was added sodium hydride (60% in mineral oil) (0.394 g, 9.87 mmol, 1.5 equiv) in portions. The reaction mixture was stirred for 30 min at 0° C. and 2-(trimethylsilyl) ethoxymethylchloride (1.206 g, 7.238 mmol, 1.1 equiv) was added. It was stirred at room temperature for 1 h, transferred into ice-water and extracted with ethyl acetate. The combined organic layers were washed with brine, dried over anhydrous sodium sulfate. The residue was purified by flash column chromatography on silica gel (CombiFlash®, 10% ethyl acetate in hexane) to afford H-28.2 (1.2 g, 49%), MS(ES): m/z 373.2 and 375.2 [M+H]⁺.

Synthesis of compound H-28.3. A mixture of compound H-28.2 (1.2 g, 3.214 mmol, 1.0 equiv), ethanol (28.8 mL), water (9.6 mL), ammonium chloride (0.859 g, 16.07 mmol, 5 equiv) and iron powder (0.897 g, 16.07 mmol, 5 equiv) was stirred at 80° C. for 90 min. The reaction mixture was filtered through a pad of Celite®. The filtrate was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford H-28.3 (0.795 g, 72%). MS(ES): m/z 343.1 and 345.1 [M+H]⁺.

Synthesis of compound H-28. To a solution of H-28.3 (0.795 g, 2.315 mmol, 1.0 equiv) in acetonitrile (8 mL) was added potassium carbonate (0.966 g, 6.945 mmol, 3.0 equiv) followed by the addition of 1-bromo-2-(2-bromoethoxy)ethane (0.536 g, 2.315 mmol, 1.0 equiv). The reaction mixture was stirred at 150° C. for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.1% methanol in DCM) to afford H-28 (0.210 g, 22%). MS(ES): m/z 414.39 [M+H]⁺.

### Preparation of Intermediate trans-H-29: trans-6-(2-((tert-butyldiphenylsilyl)oxy)cyclobutoxy)-3-iodopyrazolo[1,5-a]pyrazine

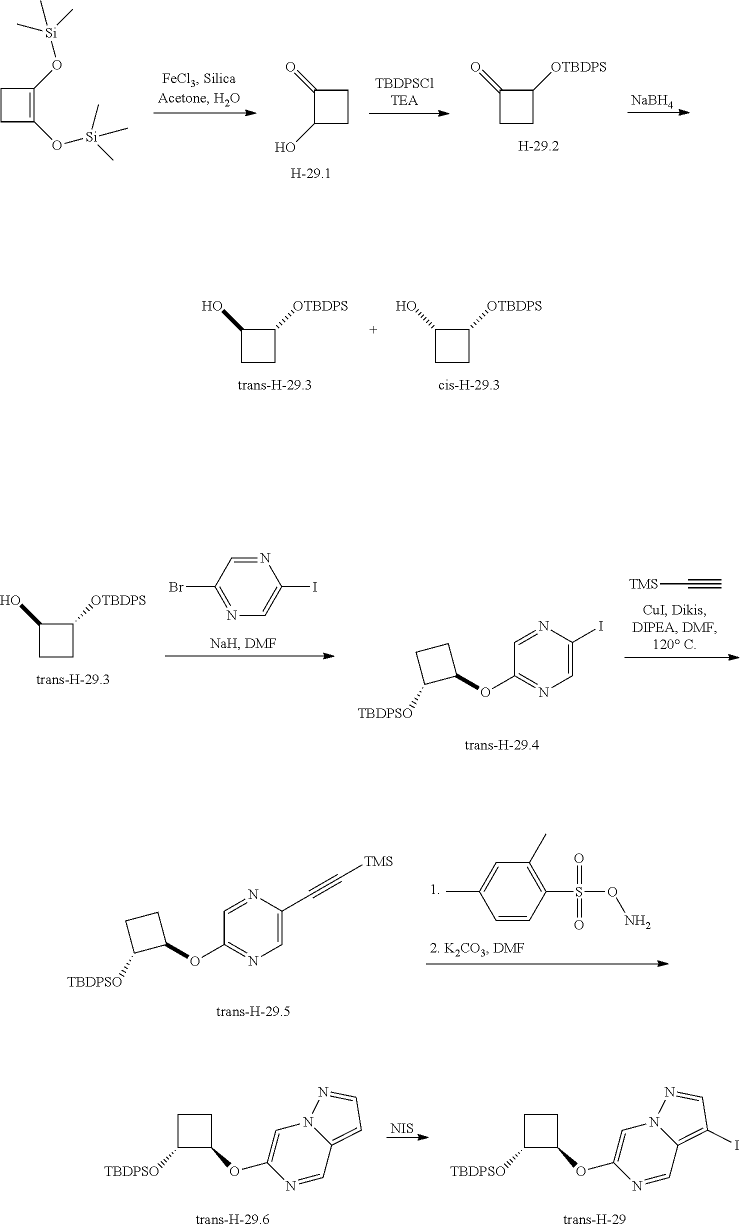

Synthesis of compound H-29.1. To a solution of 1,2-bis((trimethylsilyl)oxy)cyclobut-1-ene (10 g, 43.39 mmol, 1.0 equiv) in acetone: water (20:1, 50 mL) was added ferric chloride in silica (0.050 g, 0.5% w/w). The reaction mixture was stirred at room temperature for 1.5 h. It was concentrated under reduced pressure. The residue was purified by flash column chromatography on silica gel (CombiFlash®, 11% ethyl acetate in hexane) to afford H-29. ¹H NMR (CDCl₃, 400 MHz): δ 5.02-5.00 (m, 1H), 2.88-2.78 (m, 2H), 2.49-2.43 (m, 1H), 1.93-1.88 (m, 1H).

Synthesis of compound H-29.2. Compound H-29.1 (2.2 g, 25.55 mmol, 1.0 equiv) was added triethylamine (5.16 g, 51.1 mmol, 2.0 equiv) and stirred at room temperature for 10 min followed by addition of tert-butyldiphenylchlorosilane (14.05 g, 51.1 mmol, 2.0 equiv). The reaction mixture was stirred at room temperature for 12 h. The reaction mixture was poured over brine and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% ethyl acetate in hexane) to afford H-29.2. MS (ES): m/z 325.6 [M+H]⁺.

Synthesis of compound trans-H-29.3 and cis-H-29.3. To a solution of H-29.2 (2.2 g, 6.78 mmol, 1.0 equiv) in methanol (15 mL) was added sodium borohydride (0.501 g, 13.56 mmol, 2.0 equiv) in portions at 0° C. The reaction mixture was stirred at 0° C. for 30 min. It was poured over ice-water, stirred and extracted with ethyl acetate. The combined organic layers were washed with brine, dried over anhydrous sodium sulfate, filtered, and concentrated under reduced pressure. The residue was purified by flash column chromatography on silica gel (CombiFlash®, 8-15% ethyl acetate in hexane) to afford trans-H-29.3, MS (ES): m/z 327.1 [M+H]⁺ and cis-H-29.3. MS (ES): m/z 327.1 [M+H]⁺.

Synthesis of compound trans-H-29.4. To a solution of trans-H-29.3 (29 g, 79.5 mmol, 1.5 equiv) in DMF (150 mL) at 0° C. was added sodium hydride (60% in mineral oil) (2.3 g, 159 mmol, 3.0 equiv) followed by addition of 2-bromo-5-iodopyrazine (15 g, 53.0 mmol, 1.0 equiv). The reaction mixture was stirred at room temperature for 1 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford trans-H-29.4 (13 g, 46%). MS(ES): m/z 531.2 [M+H]⁺.

Synthesis of compound trans-H-29.5. A mixture of trans-H-29.42 (13 g, 24.52 mmol, 1.0 equiv) and N,N-diisopropylethylamine (10 mL, 73.58 mmol, 3.0 equiv) in DMF (130 mL) was degassed by bubbling through a stream of argon for 10 min. Copper(I) iodide (0.232 g, 1.226 mmol, 0.05 equiv) and bis(triphenylphosphine)palladium(II) dichloride (0.859 g, 1.226 mmol, 0.05 equiv). The reaction mixture was degassed for 15 min and was added ethynyltrimethylsilane (10 mL, 73.56 mmol, 3.0 equiv). It was stirred at 120° C. for 16 h, poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.0% methanol in DCM) to afford trans-H-29.5 (9.0 g, 76%). MS(ES): m/z: 502.1 [M+H]⁺.

Synthesis of compound trans-H-29.6. To a solution of trans-H-29.5 (9.0 g, 17.96 mmol, 1.0 equiv) in DCM (90 mL) was added O-(mesitylsulfonyl)hydroxylamine (4.6 g, 21.55 mmol, 1.2 equiv) at 0° C. The reaction mixture was stirred at room temperature for 16 h. It was concentrated under reduced pressure. The residue was dissolved in DMF (100 mL) and added potassium carbonate (8.02 g, 58.13 mmol, 2.0 equiv). The reaction mixture was stirred at room temperature for 16 h. It was transferred into brine and extracted with ethyl acetate. The combined organic layers were washed with brine, dried over anhydrous sodium sulfate, filtered, and concentrated under reduced pressure. The residue was purified by flash column chromatography on silica gel (6.0% ethyl acetate in hexane) to afford trans-H-29.6 (1.3 g, 16%). MS (ES): m/z 444.19 [M+H]⁺.

Synthesis of compound trans-H-29. To a solution of trans-H-29.6 (1.3 g, 2.93 mmol, 1.0 equiv) in acetonitrile (15 mL) was added in portions N-iodosuccinimide (0.716 g, 1.25 mmol, 1.5 equiv) at 0° C. The reaction mixture was stirred at room temperature for 6 h. It was concentrated under reduce pressure. To the residue was added water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford trans-H-29 (0.84 g, 65%). MS(ES): m/z 570.10 [M+H]⁺.

### Preparation of Intermediate trans-H-31: N-(5-bromo-4-methoxypyridin-2-yl)cyclopropanecarboxamide

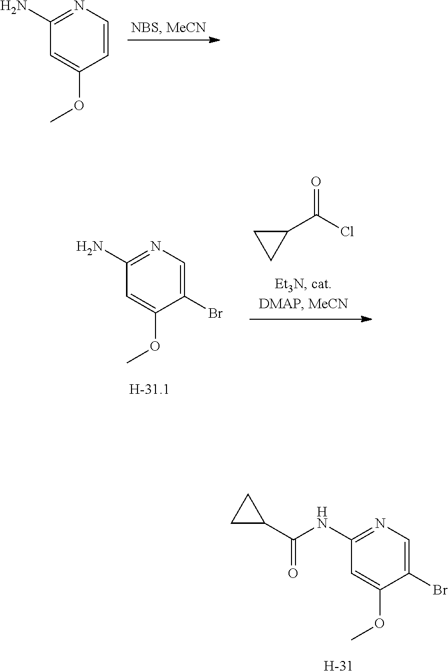

Synthesis of compound H-31.1. To a solution of 4-methoxypyridin-2-amine (5.0 g, 40.32 mmol, 1.0 equiv) in acetonitrile (200 mL) was added N-bromosuccinimide (7.17 g, 40.32 mmol, 1.0 equiv) in portions at 0° C. The reaction mixture was stirred at room temperature for 1 h. It was concentrated under vacuum pressure to remove acetonitrile. The residue afford was dissolved with DCM and washed with saturated bicarbonate solution. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford H-31.1 (3.1 g, 38%). MS(ES): m/z 204.04 [M+H]⁺.

Synthesis of compound H-31. To a solution of H-31.1 (1.6 g, 40.32 mmol, 1.0 equiv) in acetonitrile (200 mL) was added cyclopropanecarbonyl chloride (1.639 g, 15.763 mmol, 2.0 equiv) at 0° C. followed by addition of triethylamine (7.17 g, 40.32 mmol, 1.0 equiv) and 4-dimethylaminopyridine (0.009 g, 0.079 mmol, 0.01 equiv). The mixture was stirred at room temperature for 1 h. It was poured over ice water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford H-31 (1.0 g, 47%). MS(ES): m/z 272.11 [M+H]⁺.

### Preparation of Intermediate H-33: benzyl (2-bromo-5-((2-(trimethylsilyl)ethoxy)methyl)-5H-pyrrolo[2,3-b]pyrazin-7-yl)(methyl)carbamate

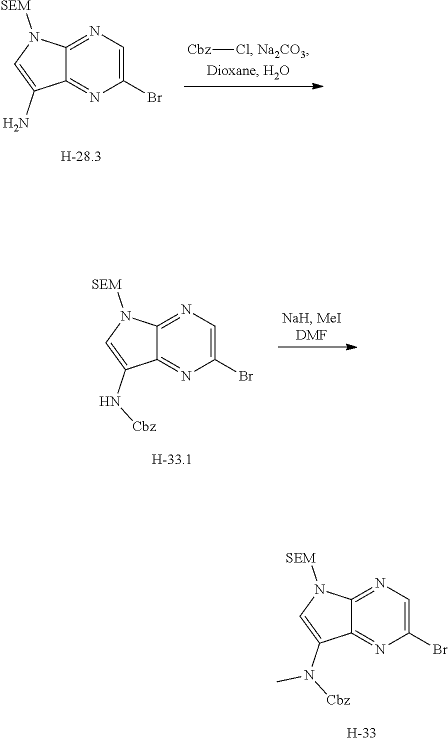

Synthesis of compound H-33.1. To a solution of H-28.3 (2 g, 5.83 mmol, 1.0 equiv) in dioxane (30 mL) was added saturated sodium carbonate solution in water (20 mL) at 0° C. and stirred for 10 min. To the mixture was added benzyl chloroformate (50% in toluene, 3.9 mL, 11.66 mmol, 2 equiv) at 0° C. The reaction mixture was stirred for 30 min. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford H-31.1 (2.2 g, 79%). MS(ES): m/z 477.28 and 479.28 [M+H]⁺.

Synthesis of compound H-33. To a solution of compound H-33.1 (2.2 g, 4.61 mmol, 1.0 equiv) in DMF (20 mL) was added sodium hydride (0.36 g, 9.2 mmol, 2 equiv) in portions and stirred for 30 min at 0° C. To the mixture was added dropwise methyl iodide (1.3 g, 9.2 mmol, 2 equiv). The reaction mixture was stirred room temperature for 30 min. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 22% ethyl acetate in hexane) to afford H-33 (1.9 g, 84%). MS(ES): m/z 491.46 and 493.4 [M+H]⁺.

### Preparation of Intermediate H-35: 5-bromo-3-(4-methylpiperazin-1-yl)-1-((2-(trimethylsilyl)ethoxy)methyl)-1H-pyrazolo[3,4-b]pyridine

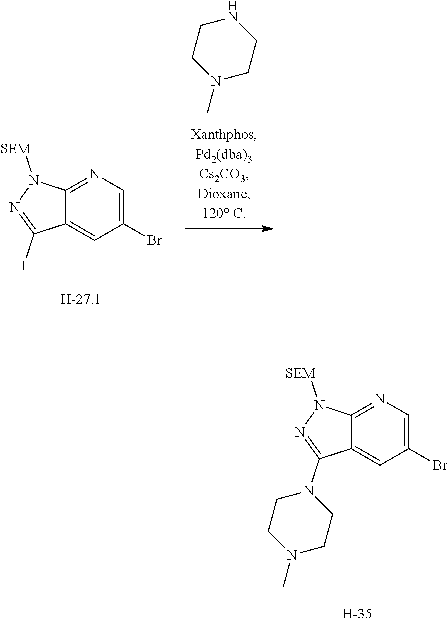

Synthesis of compound H-35. A mixture of H-27.1 (2.0 g, 4.45 mmol, 1.0 equiv), 1-methylpiperazine (0.663 g, 6.63 mmol, 1.5 equiv), cesium carbonate (1.13 g, 13.27 mmol, 3.0 equiv), 9,9-dimethyl-4,5-bis(diphenylphosphino)xanthene (0.767 g, 1.327 mmol, 0.3 equiv) and tris(dibenzylideneacetone)dipalladium (0) (0.32 g, 0.88 mmol, 0.2 equiv) in 1,4-dioxane (20 mL) was purged with argon for 10 min. The reaction mixture was stirred at 120° C. for 2 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.8% methanol in DCM) to afford H-35 (0.900 g, 50%). MS(ES): m/z 426.4 and 428.4 [M+H]⁺.

### Preparation of Intermediate H-36: 5-bromo-3-methoxy-1-((2-(trimethylsilyl)ethoxy)methyl)-1H-pyrazolo[3,4-b]pyridine

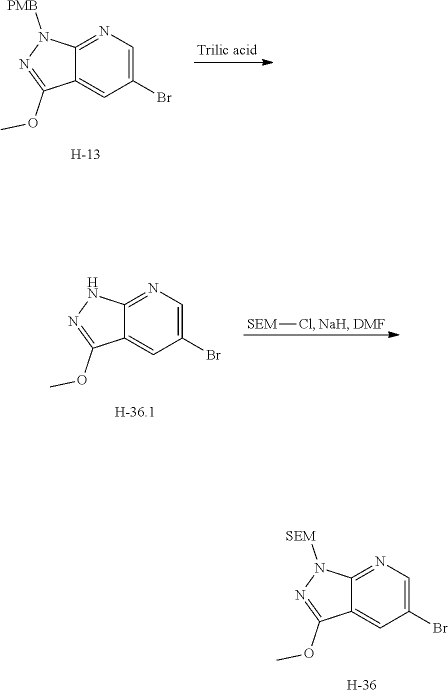

Synthesis of compound H-36.1. To a solution of H-13 (2.0 g, 5.74 mmol, 1.0 equiv) in DCM (30 mL) was added trifluoroacetic acid (5 mL) at 0° C. The reaction mixture was stirred at room temperature for 3 h. The mixture was washed with a saturated aqueous solution of sodium bicarbonate and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.8% methanol in DCM) to afford H-36.1 (1.2 g, 92%). MS(ES): m/z 228.1 and 230.1 [M+H]⁺.

Synthesis of compound H-36. Compound H-36 was prepared from H-36.1 following the procedure described in the synthesis of H-1. The product was purified by flash column chromatography on silica gel (CombiFlash®, 18% ethyl acetate in hexane). MS (ES): m/z 358.21 and 360.2 [M+H]⁺.

### Preparation of Intermediate H-37: 5-bromo-3-(2-methoxyethoxy)-1-((2-(trimethylsilyl)ethoxy)methyl)-1H-pyrazolo[3,4-b]pyridine

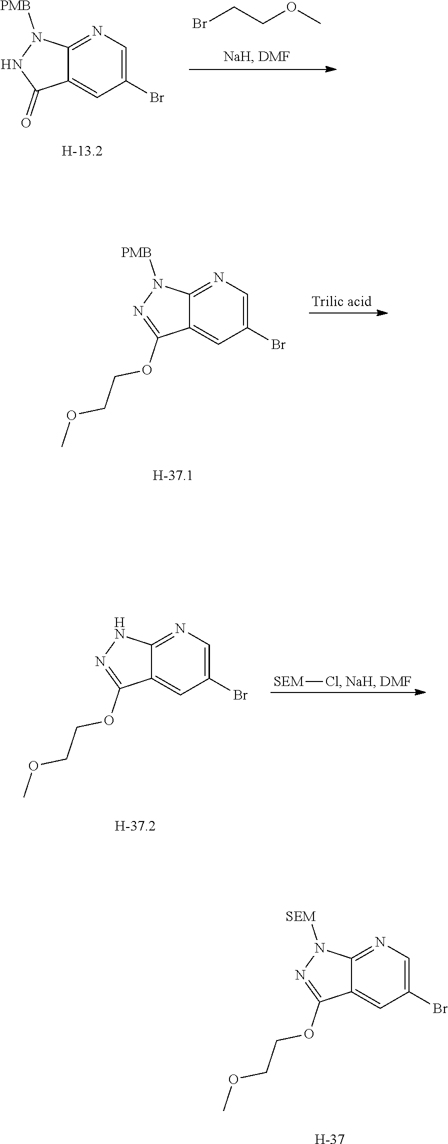

Synthesis of compound H-37.1. To a solution of H-13.2 (3.5 g, 10.47 mmol, 1.0 equiv) in DMF (35 mL) at 0° C. was added sodium hydride (60%) (1.0 g, 20.94 mmol, 2.0 equiv) and stirred for 15 min. To the mixture was added 1-bromo-2-methoxyethane (1.73 g, 12.57 mmol, 1.2 equiv) and stirred at room temperature for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (18% ethyl acetate in hexane) to afford H-37.1 (2.90 g, 66%). MS(ES): m/z 394.2 and 396.2 [M+H]⁺.

Synthesis of compound H-37.2. To a solution of H-37.1 (1.8 g, 4.59 mmol, 1.0 equiv) in DCM (20 mL) was added trifluoromethanesulfonic acid (5 mL) at 0° C. The reaction mixture was stirred at room temperature for 3 h. The mixture was washed with saturated sodium bicarbonate and the aqueous was extracted with DCM. The combined organic layers were washed with brine, dried over anhydrous sodium sulfate, filtered, and concentrated under reduced pressure. The residue was purified by flash column chromatography on silica gel (CombiFlash®, 2.1% methanol in DCM) to afford H-37.2 (0.98 g, 78%). MS(ES): m/z 274.1 and 276.1 [M+H]⁺.

Synthesis of compound H-37. Compound H-37 was prepared from H-37.2 following the procedure described in the synthesis of H-1. The product was purified by flash column chromatography on silica gel (CombiFlash®, 32% ethyl acetate in hexane). MS (ES): m/z 404.3 and 406.3 [M+H]⁺.

### Preparation of Intermediate H-38: (R)-3-((1,4-dioxan-2-yl)methoxy)-5-bromo-1-((2-(trimethylsilyl)ethoxy)methyl)-1H-pyrazolo[3,4-b]pyridine

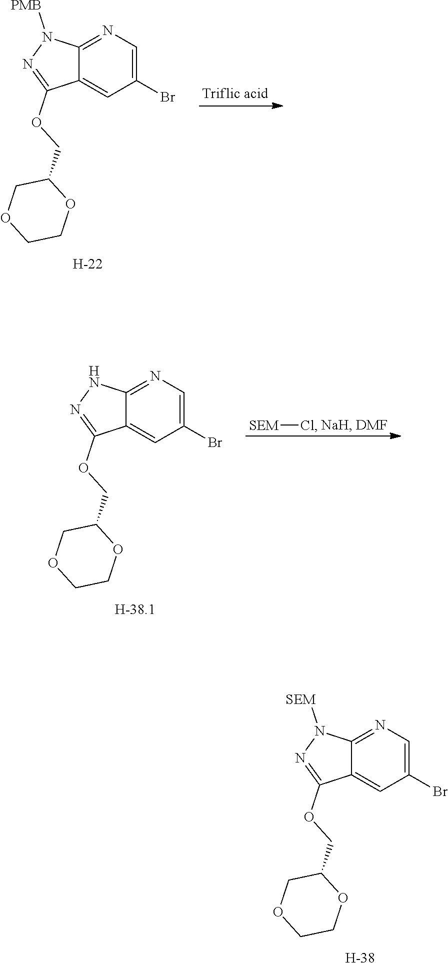

Synthesis of compound H-38.1. To a solution of H-22 (2.0 g, 4.61 mmol, 1.0 equiv) in DCM (20 mL) was added trifluoromethanesulfonic acid (5 mL) at 0° C. The reaction mixture was stirred at room temperature for 3 h. The mixture was washed with saturated sodium bicarbonate and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.2% methanol in DCM) to afford H-38.1 (1.1 g, 76%). MS(ES): m/z 314.1 and 316.1 [M+H]⁺.

Synthesis of compound H-38. Compound H-38 was prepared from H-38.1 following the procedure described in the synthesis of H-1. The product was purified by flash column chromatography on silica gel (CombiFlash®, 30% ethyl acetate in hexane). MS (ES): m/z 444.5 and 446.5 [M+H]⁺.

### Preparation of Intermediate H-39: 5-bromo-3-((tetrahydrofuran-3-yl)oxy)-1-((2-(trimethylsilyl)ethoxy)methyl)-1H-pyrazolo[3,4-b]pyridine

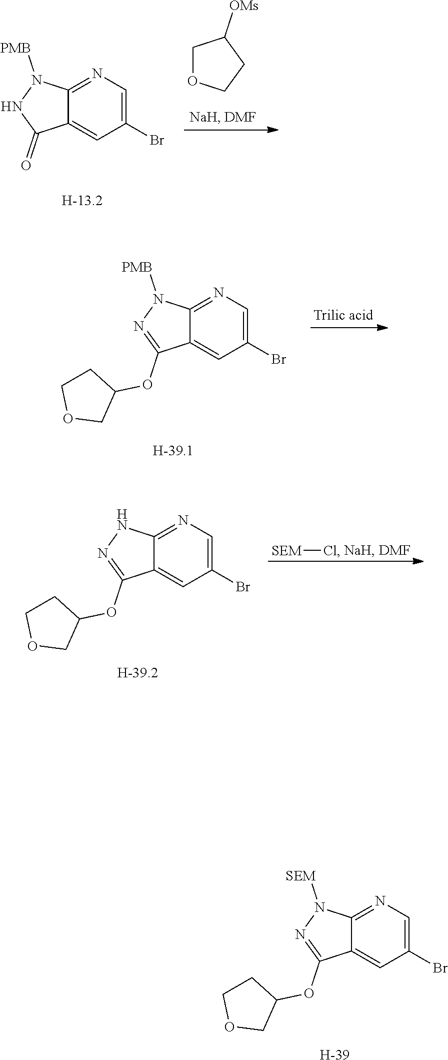

Synthesis of compound H-39.1. To a solution of H-13.2 (3.5 g, 10.47 mmol, 1.0 equiv) in DMF (35 mL) at 0° C. was added sodium hydride (60 wt %, 1.0 g, 20.94 mmol, 2.0 equiv) and stirred for 15 min. To the mixture was added tetrahydrofuran-3-yl methanesulfonate (2.09 g, 12.57 mmol, 1.2 equiv) and stirred at room temperature for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (15% ethyl acetate in hexane) to afford H-39.1 (2.70 g, 64%). MS(ES): m/z 404.2 and 406.2 [M+H]⁺.

Synthesis of compound H-39.2. To a solution of H-39.1 (2.0 g, 4.95 mmol, 1.0 equiv) in DCM (20 mL) was added trifluoroacetic acid (5 mL) at 0° C. The reaction mixture was stirred at room temperature for 3 h. The mixture was washed with a saturated aqueous solution of sodium bicarbonate and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.2% methanol in DCM) to afford H-39.2 (1.3 g, 92%). MS(ES): m/z 284.1 and 286.1 [M+H]⁺.

Synthesis of compound H-39. Compound H-39 was prepared from H-39.2 following the procedure described in the synthesis of H-1. The product was purified by flash column chromatography on silica gel (CombiFlash®, 28% ethyl acetate in hexane). MS (ES): m/z 414.2 and 416.2 [M+H]⁺.

### Preparation of Intermediate H-40: 5-bromo-3-nitro-1-((2-(trimethylsilyl)ethoxy)methyl)-1H-pyrrolo[2,3-b]pyridine

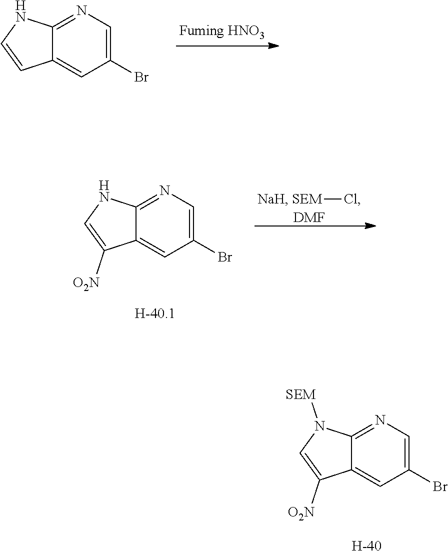

Synthesis of compound H-40.1 A solution of 5-bromo-1H-pyrrolo[2,3-b]pyridine (10.0 g, 50.76 mmol, 1.0 equiv) in fuming HNO₃ (33 mL) was stirred at 0° C. for 1 h. It was poured into ice-water. The solids precipitated were collected by filtration, rinsed with water and dried under vacuum to afford H-40.1 (8.0 g, 65%). MS(ES): m/z 243.23 [M+H]⁺.

Synthesis of compound H-40. Compound H-40 was prepared from H-40.1 following the procedure described in the synthesis of H-1. The product was purified by flash column chromatography on silica gel (CombiFlash®, 30% ethyl acetate in hexane). MS(ES): m/z 373.42 [M+H]⁺.

### Preparation of Intermediate L-1: N-benzyl-3,3-difluoro-N-methylpiperidin-4-amine

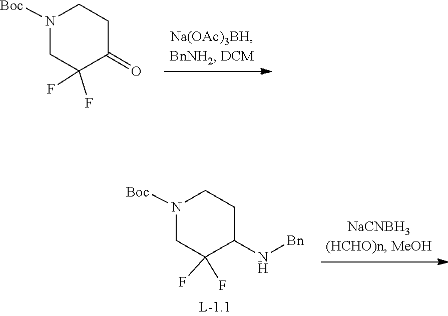 [SMILES: CC(C)(C)OC(=O)N1CCC(=O)C(F)(F)C1 ; CC(C)(C)OC(=O)N1CCC(NCc2ccccc2)C(F)(F)C1]

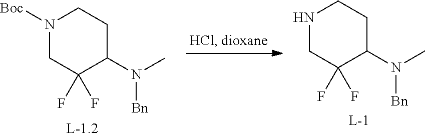 [SMILES: CN(Cc1ccccc1)C1CCN(C(=O)OC(C)(C)C)CC1(F)F ; CN(Cc1ccccc1)C1CCNCC1(F)F]

Synthesis of compound L-1.1. To a solution of tert-butyl 3,3-difluoro-4-oxopiperidine-1-carboxylate (5 g, 21.25 mmol, 1.0 equiv) in DCM (100 mL) was added benzyl amine (3.41 g, 31.88 mmol, 1.5 equiv) and sodium triacetoxyborohydride (18 g, 85.02 mmol, 4.0 equiv). The reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford L-1.1 (3.9 g, 56%). MS(ES): m/z 327.1 [M+H]⁺.

Synthesis of compound L-1.2. To a solution of L-1.1 (3.8 g, 11.62 mmol, 1.0 equiv) and paraformaldehyde (3.48 g, 116.2 mmol, 10 equiv) in methanol (75 mL) was added sodium cyanoborohydride (3.65 g, 58.10 mmol, 5.0 equiv). The reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 28% ethyl acetate in hexane) to afford (+)-22.2 (3.1 g, 78%). MS(ES): m/z 341.1 [M+H]⁺.

Synthesis of compound L-1. To a solution of L-1.2 (3.1 g, 2.71 mmol, 1.0 equiv) in methanol (31 mL) was added hydrochloric acid (4.0 M in dioxane, 30 mL). The reaction mixture was stirred at room temperature for 3 h. It was transferred into saturated sodium bicarbonate solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 4% methanol in DCM) to afford L-1 (1.6 g, 73%). MS(ES): m/z 241.1 [M+H]⁺.

### Preparation of Intermediate L-2: N-benzyl-N-methylazepan-4-amine

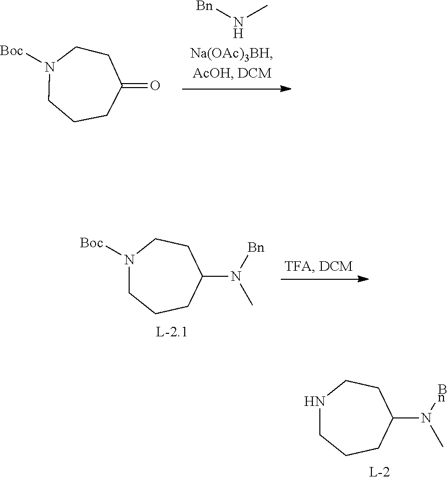

Synthesis of compound L-2.1. To a solution of tert-butyl 4-oxoazepane-1-carboxylate (2.0 g, 9.38 mmol, 1.0 equiv), N-benzylmethylamine (1.36 g, 11.26 mmol, 1.2 equiv) and acetic acid (0.619 g, 10.32 mmol, 1.2 equiv) in DCM (20 mL) was added sodium triacetoxyborohydride (2.98 g, 14.08 mmol, 1.5 equiv) at 0° C. and stirred at room temperature for 20 h. It was transferred into a saturated solution of sodium bicarbonate (50 mL) and extracted with DCM. The combined organic layers were washed with brine, dried over anhydrous sodium sulfate, filtered, and concentrated under reduced pressure. The residue was purified by flash column chromatography on silica gel (CombiFlash®, 2.8% methanol in DCM) to afford L-2.1 (1.9 g, 64%). MS(ES): m/z: 319.2 [M+H]⁺.

Synthesis of compound L-2. To a solution of L-2.1 (1.8 g, 5.66 mmol, 1.0 equiv) in DCM (20 mL) was added trifluoroacetic acid (10 mL) at 0° C. and stirred for 1.5 h. It was transferred into ice-cold saturated sodium bicarbonate solution and extracted with DCM. The combined organic layers were washed with brine, dried over anhydrous sodium sulfate, filtered, and concentrated under reduced pressure. The residue was purified by trituration with diethyl ether to afford L-2 (1.17 g, 95%). MS(ES): m/z: 219.2 [M+H]⁺.

### Preparation of Intermediate L-3: benzyl ((1S,4S,5R)-2-azabicyclo[2.2.1]heptan-5-1 (meth 1)carbamate

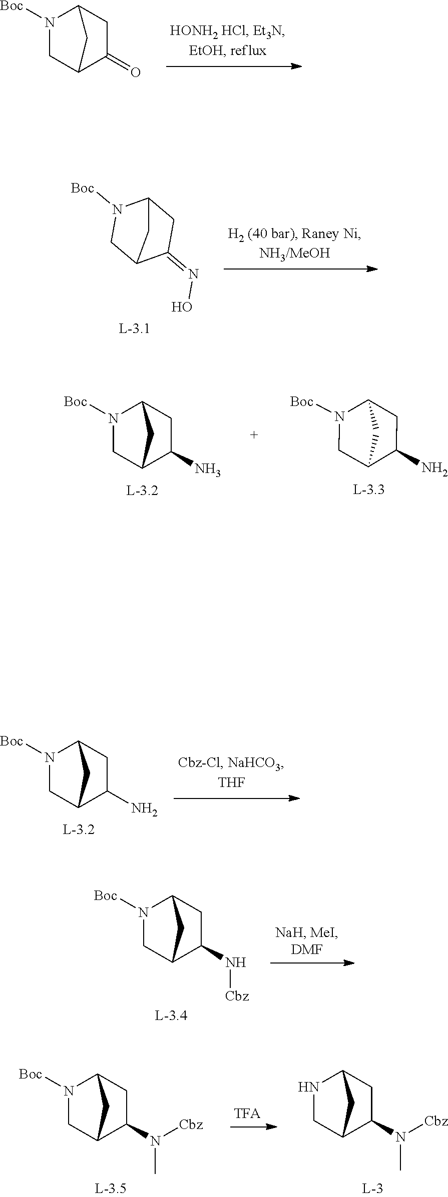

Synthesis of compound L-3.1. To a solution of triethylamine (5.9 g, 42.65 mmol, 0.9 equiv) and hydroxylamine hydrochloride (3.6 g, 52.13 mmol, 1.1 equiv) at 0° C. was added compound tert-butyl 5-oxo-2-azabicyclo[2.2.1]heptane-2-carboxylate (10 g, 47.39 mmol, 1.0 equiv) in ethanol (0.619 g, 10.32 mmol, 1.2 equiv). The reaction mixture was stirred at 80° C. for 2 h. It was concentrated under reduced pressure. The residue was triturated with water, filtered and dried to afford L-3.1 (7.0 g, 65%). MS(ES): m/z: 227.13 [M+H]⁺.

Synthesis of compound L-3.2. and L-3.3. A mixture of compound L-3.1 (7.0 g, 30.83 mmol, 1.0 equiv), 10% methanolic ammonia (80 mL) and Raney nickel (5.0 g) in a Paar shaker was shaken under 40 bars hydrogen for 8 h. The reaction mixture was filtered through a pad of Celite® and rinsed with methanol. The filtrate was concentrated under reduced pressure. The residue was separated by flash column chromatography on silica gel (CombiFlash®, 2-propanol) to afford L-3.2 (2.1 g, 32%). MS(ES): m/z 214.16 [M+H]⁺ and L-3.3 (3.3 g, 50%). MS(ES): m/z 214.16 [M+H]⁺.

Synthesis of compound L-3.4. To a solution of L-3.2 (2.1 g, 9.90 mmol, 1.0 equiv) and sodium bicarbonate (2.5 g, 29.71 mmol, 3.0 equiv) in THE (40 mL) and water (20 mL) at 0° C. was added benzyl chloroformate (1.4 mL, 9.90 mmol, 1.0 equiv) dropwise. The reaction mixture was stirred at room temperature for 6 h. It was transferred into water and extracted with DCM. The combined organic layers were washed with brine, dried over anhydrous sodium sulfate, filtered, and concentrated under reduced pressure. The residue was purified by flash column chromatography on silica gel (CombiFlash®, 18% ethyl acetate in hexane) to afford L-3.4 (1.6 g, 47%). MS(ES): m/z: 347.19 [M+H]⁺.

Synthesis of compound L-3.5. To a solution of L-3.4 (1.6 g, 4.62 mmol, 1.0 equiv) in DMF (15 mL) was added sodium hydride (60 wt %, 0.182 g, 6.93 mmol, 1.5 equiv) in portions and stirred for 15 min at 0° C. To the mixture was added methyl iodide (0.6 mL, 13.86 mmol, 3.0 equiv) and the mixture was allowed to warm to rt and stirred for 2 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-3.5 (1.0 g, 60%). MS(ES): m/z 361.20 [M+H]⁺.

Synthesis of compound L-3. To a solution of L-3.5 (0.6 g, 1.66 mmol, 1.0 equiv) in DCM (1.2 mL) was added trifluroacetic acid (0.6 mL). The reaction mixture was stirred at room temperature for 1 h. It was transferred into a saturated sodium bicarbonate solution and extracted with DCM. The combined organic layers were washed with brine, dried over anhydrous sodium sulfate, filtered, and concentrated under reduced pressure to afford L-3 (0.421 g, 97%). MS(ES): m/z 261.15 [M+H]⁺.

### Preparation of Intermediate L-4: tert-butyl (3,3-dimethylpiperidin-4-yl)(methyl)carbamate

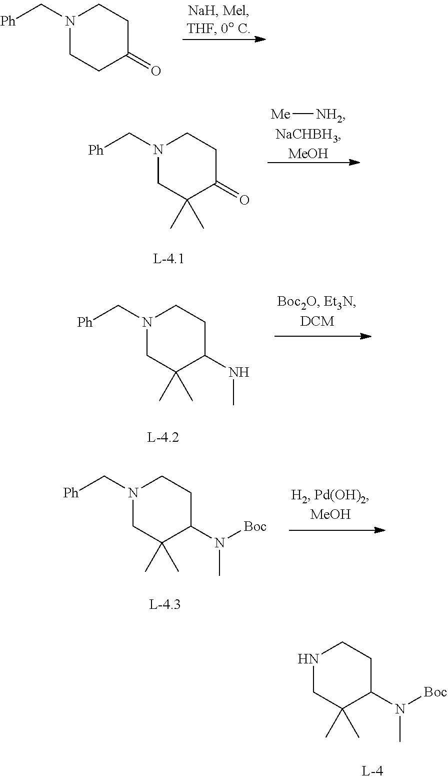

Synthesis of compound L-4.1 To a solution of 1-benzylpiperidin-4-one (5.0 g, 26.45 mmol, 1.0 equiv) in THE (40 mL) at 0° C. was added sodium hydride (1.0 g, 38.17 mmol, 1.5 equiv) and stirred for 30 min. To the mixture was added iodomethane (1.97 mL, 31.74 mmol, 1.2 equiv) dropwise. It was stirred at room temperature for 12 h, transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-4.1 (1.62 g, 28%). MS(ES): m/z 218.15 [M+H]⁺.

Synthesis of compound L-4.2. To a solution of L-4.1 (1.62 g, 7.46 mmol, 1.0 equiv) in methanol (20 mL) was added methylamine hydrochloride (5.0 g, 74.6 mmol, 10 equiv) and potassium hydroxide (4.1 g, 74.6 mmol, 10 equiv). The mixture was stirred at room temperature for 6 h and was added sodium cyanoborohydride (0.470 g, 7.46 mmol, 1.0 equiv) in portions. The reaction mixture was stirred at room temperature for 12 h. It was transferred into water and extracted with DCM. The combined organic layers were washed with brine, dried over anhydrous sodium sulfate, filtered, and concentrated under reduced pressure to afford L-4.2 (1.1 g, 64%). MS(ES): m/z 233.19 [M+H]⁺.

Synthesis of compound L-4.3. To a solution of L-4.2 (1.1 g, 4.74 mmol, 1.0 equiv) in DCM (10 mL) was added triethylamine (1.3 mL, 11.85 mmol, 2.5 equiv) followed by addition of di-tert-butyl dicarbonate (1.4 mL, 11.85 mmol, 1.5 equiv) at 0° C. The mixture was stirred at room temperature for 1 h. It was transferred into ice-water and extracted with DCM. The combined organic layers were washed with brine, dried over anhydrous sodium sulfate, filtered, and concentrated under reduced pressure to afford L-4.3 (0.830 g, 53%). MS (ES): m/z 333.25 [M+H]⁺.

Synthesis of compound L-4. A mixture of compound L-4.3 (0.83 g, 2.50 mmol, 1.0 equiv) and 10% palladium hydroxide on carbon (0.1 g) in methanol (20 mL) was stirred under hydrogen (1 atm) at room temperature for 2 h. The reaction mixture was filtered through a pad of Celite® and rinsed with methanol. The filtrate was concentrated under reduced pressure to afford L-4 (0.55 g, 91%). MS(ES): m/z 243.2 [M+H]⁺.

### Preparation of Intermediate L-5: N-benzyl-N-methylpiperidin-4-amine hydrochloride

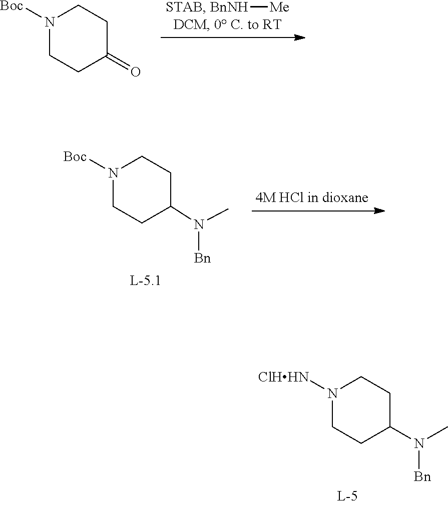

Synthesis of compound L-5.1. To a solution of tert-butyl 4-oxopiperidine-1-carboxylate (2.0 g, 10.03 mmol, 1.0 equiv) in DCM (20 mL) was added N-benzylmethylamine (1.45 g, 12.04 mmol, 1.2 equiv) and stirred at room temperature for 6 h. To the mixture was added sodium triacetoxyhydroborate (2.1 g, 10.03 mmol, 1.0 equiv) in portions. The reaction mixture was stirred at room temperature for 16 h. It was transferred into water and extracted with DCM. The combined organic layers were washed with brine, dried over anhydrous sodium sulfate, filtered, and concentrated under reduced pressure to afford L-5.1 (0.8 g, 26%). MS(ES): m/z 305.22 [M+H]⁺.

Synthesis of compound L-5. To a solution of L-5.1 (0.8 g, 2.63 mmol, 1.0 equiv) in DCM (0.8 mL) was added 4.0 M hydrochloric acid in dioxane (0.8 mL). The reaction mixture was stirred at room temperature for 2 h. It was concentrated under reduced pressure. The residue was azeotroped with DCM to afford L-5 (0.65 g). MS(ES): m/z 205.14 [M+H]⁺.

### Preparation of Intermediate L-6: N-benzyl-N,3,3-trimethylpiperidin-4-amine

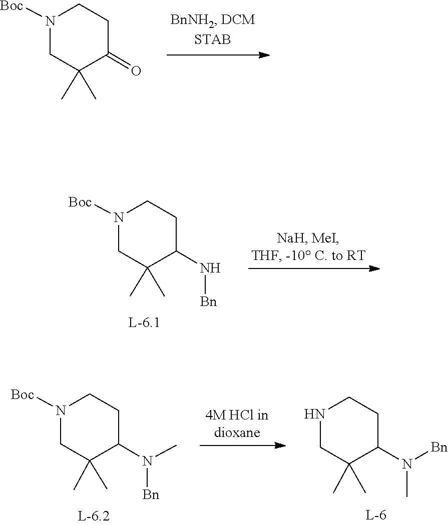 [SMILES: CC(C)(C)OC(=O)N1CCC(=O)C(C)(C)C1 ; CC(C)(C)OC(=O)N1CCC(NCc2ccccc2)C(C)(C)C1 ; CN(Cc1ccccc1)C1CCN(C(=O)OC(C)(C)C)CC1(C)C ; CN(Cc1ccccc1)C1CCNCC1(C)C]

Synthesis of compound L-6.1. To a solution of tert-butyl 3,3-dimethyl-4-oxopiperidine-1-carboxylate (4.5 g, 19.80 mmol, 1.0 equiv) in DCM (100 mL) was added benzyl amine (3.10 g, 29.7 mmol, 1.5 equiv) and sodium triacetoxy borohydride (16.7 g, 79.2 mmol, 4.0 equiv). The reaction mixture was stirred at room temperature for 16 h. It was transferred into water and extracted with DCM. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford L-6.1 (3.1 g, 49%). MS(ES): m/z 319.23 [M+H]⁺.

Synthesis of compound L-6.2. To a solution of L-6.1 (3.1 g, 9.73 mmol, 1.0 equiv) in THF (30 mL) was added sodium hydride (0.467 g, 19.46 mmol, 2.0 equiv) at −10° C. followed by dropwise addition of methyl iodide (2.0 g, 14.59 mmol, 1.5 equiv). The reaction mixture was stirred at room temperature for 6 h. It transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 28% ethyl acetate in hexane) to afford L-6.2 (2.5 g, 77%). MS(ES): m/z 333.49 [M+H]⁺.

Synthesis of compound L-6. To a solution of L-6.2 (2.5 g, 10.7 mmol, 1.0 equiv) in DCM (25 mL) was added 4.0 M hydrochloric acid in dioxane (25 mL). The reaction mixture was stirred at room temperature for 3 h. It was transferred into a saturated sodium bicarbonate solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3% methanol in DCM) to afford L-6 (1.8 g). MS(ES): m/z 233.37 [M+H]⁺. It was used without purification.

### Preparation of Intermediate L-7: benzyl 3-methyl-1,8-diazaspiro[4.5]decane-1-carboxylate

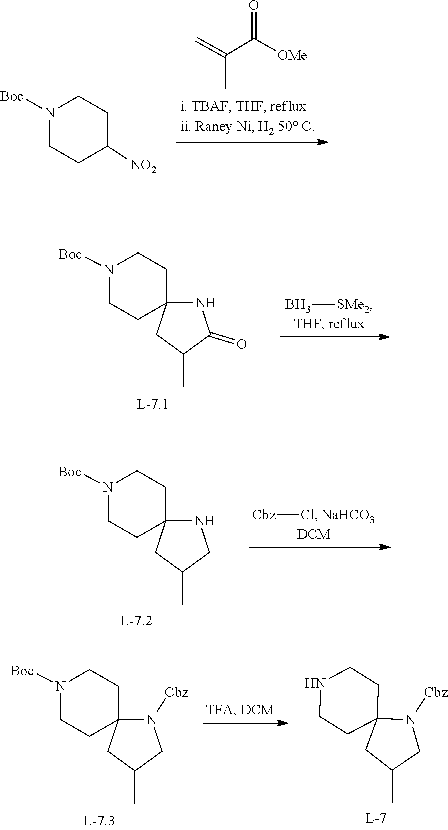

Synthesis of compound L-7.1. To a solution of tert-butyl 4-nitropiperidine-1-carboxylate (10 g, 43.47 mmol, 1.0 equiv) in THF (100 mL) at 0° C. was added methyl methacrylate (8.7 g, 86.95 mmol, 2.0 equiv) and tetra-n-butyl ammonium fluoride (52.16 mL, 52.16 mmol, 1.2 equiv). The mixture was stirred at 80° C. for 16 h. It was transferred into water and extracted with ethyl acetate. The combined organic layers were washed with brine, dried over anhydrous sodium sulfate, filtered, and concentrated under reduced pressure. The crude material was added to ethanol (100 mL) and Raney nickel (10 g) in an autoclave and stirred at 80° C. under hydrogen (20 psi) for 16 h. The reaction mixture was filtered through a pad of Celite® and washed with 50% methanol in DCM. The filtrate was concentrated under reduced pressure. The residue was purified by flash column chromatography on silica gel (CombiFlash®, 2.0% methanol in DCM) to afford L-7.1 (7.0 g, 55%). MS(ES): m/z 269.18 [M+H]⁺.

Synthesis of compound L-7.2. To a solution of L-7.1 (7.0 g, 26.10 mmol, 1.0 equiv) in THF (70 mL) was added borane dimethyl sulfide (19.8 g, 261.19 mmol, 10 equiv) at 0° C. and stirred at 80° C. for 5 h. The reaction mixture was cooled to 0° C. and quenched by adding methanol and stirred for 10 min. It was poured into water and extracted with DCM. The combined organic layers were washed with brine, dried over anhydrous sodium sulfate, filtered, and concentrated under reduced pressure to afford L-7.2 (1.3 g, 20%). MS(ES): m/z 255.20 [M+H]⁺.

Synthesis of compound L-7.3. To a solution of L-7.2 (1.3 g, 5.11 mmol, 1.0 equiv) in DCM (20 mL) was added saturated bicarbonate solution (10 mL) and benzylchloroformate (0.9 mL, 6.13 mmol, 1.2 equiv). The reaction mixture was stirred at room temperature for 6 h. It was transferred into ice-water and extracted with DCM. The combined organic layers were washed with brine, dried over anhydrous sodium sulfate, filtered, and concentrated under reduced pressure. The residue was purified by flash column chromatography on silica gel (CombiFlash®, 16% ethyl acetate hexane) to afford L-7.3 (1.0 g, 50%). MS(ES): m/z 389.24 [M+H]⁺.

Synthesis of compound L-7. To a solution of L-7.3 (1.0 g, 2.57 mmol, 1.0 equiv) in DCM (10 mL) was added trifluoroacetic acid (5.0 mL). The reaction mixture was stirred at room temperature for 2 h. It was concentrated under reduced pressure. To the residue was added water, saturated solution of bicarbonate and extracted with DCM. The combined organic layers were washed with brine, dried over anhydrous sodium sulfate, filtered, and concentrated under reduced pressure to afford L-7 (0.68 g, 92%). MS(ES): m/z 289.18 [M+H]⁺.

### Preparation of Intermediate L-8: benzyl methyl(2-azaspiro[3.3]heptan-6-yl)carbamate

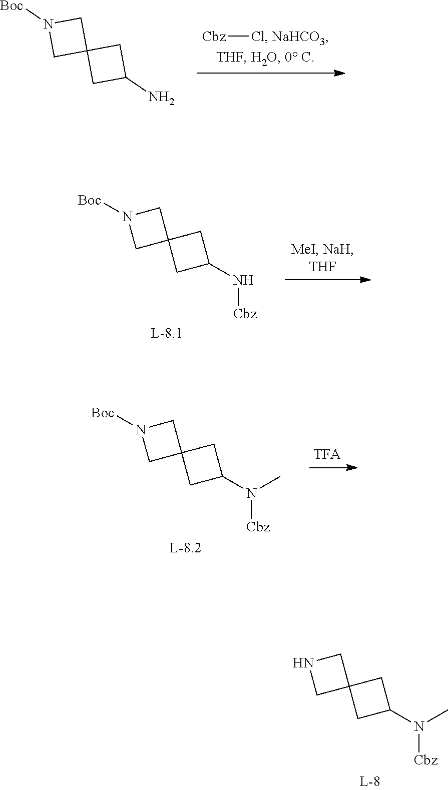

Synthesis of compound L-8.1 To a solution of tert-butyl 6-amino-2-azaspiro[3.3]heptane-2-carboxylate (2.5 g, 11.77 mmol, 1.0 equiv) in THF (20 mL) and water (20 mL) was added sodium bicarbonate (1.9 g, 23.55 mmol, 2.0 equiv) at 0° C. Benzyl chloroformate (1.9 mL, 11.77 mmol, 1.0 equiv) was added dropwise and the reaction mixture was stirred at room temperature for 2 h. It was transferred into a saturated solution of sodium bicarbonate (50 mL) and extracted with DCM. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-8.1 (2.0 g, 49%). MS(ES): m/z: 347.19 [M+H]⁺.

Synthesis of compound L-8.2. To a solution of L-8.1 (2.0 g, 5.78 mmol, 1.0 equiv) in THE (20 mL) at 0° C. was added sodium hydride (60 wt %, 0.228 g, 8.67 mmol, 1.5 equiv) and stirred for 15 min. To the mixture was added methyl iodide (1.0 mL, 17.34 mmol, 3.0 equiv). It was stirred at room temperature for 2 h, transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford L-8.2 (1.5 g, 72%). MS(ES): m/z 361.20 [M+H]⁺.

Synthesis of compound L-8. To a solution of L-8.2 (1.5 g, 4.16 mmol, 1.0 equiv) in DCM (30 mL) at 0° C. was added trifluoroacetic acid (10 mL). The reaction mixture was stirred at room temperature for 2 h. It was concentrated to remove excess trifluroacetic acid and azeotrope using DCM to afford L-8 (1.0 g, 93%). MS(ES): m/z: 261.15 [M+H]⁺.

### Preparation of Intermediate cis-L-9: cis-tert-butyl methyl(3-methylpiperidin-4-yl)carbamate

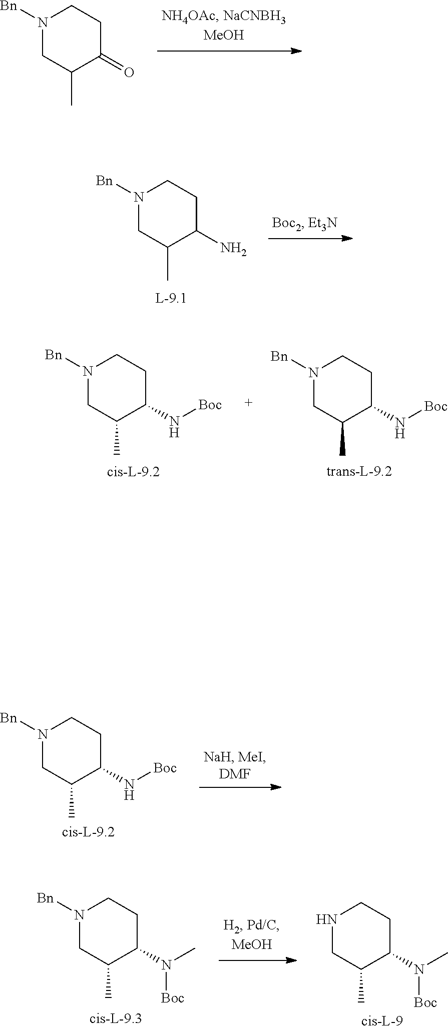

Synthesis of compound L-9.1. To a solution of 1-benzyl-3-methylpiperidin-4-one (15 g, 73.89 mmol, 1.0 equiv) in methanol (500 mL) was added ammonium acetate (56.8 g, 738.9 mmol, 10 equiv) at 0° C. and stirred at room temperature for 4 h. To the mixture was added sodium cyanoborohydride (2.32 g, 36.91 mmol, 0.5 equiv) and the reaction mixture was stirred at room temperature for 3 h. It was concentrated under reduced pressure. To the residue was added water, basified (pH 9) using 10% ammonia solution and extracted with chloroform. The combined organic layers were washed with brine, dried over anhydrous sodium sulfate, filtered, and concentrated under reduced pressure to afford L-9.1 (16 g, 99%). MS (ES: m/z 205.16 [M+H]⁺.

Synthesis of compound cis-L-9.2 and trans-L-9.2. To a solution of L-9.1 (16 g, 78.43 mmol, 1.0 equiv) in DCM (100 mL) was added trimethylamine (2.23 mL, 15.56 mmol, 0.2 equiv) and di-tert-butyl dicarbonate (17 mL, 78.43 mmol, 1 equiv) dropwise at 0° C. The reaction mixture was stirred at room temperature for 1.5 h. It was quenched with water (60 mL) and extracted with DCM (200 mL). The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford separate isomers cis-L-9.2 (5.3 g, 22%). MS(ES): m/z 305.2 [M+H]⁺ and trans-L-9.2 (2.0 g, 8%). MS(ES): m/z 305.2 [M+H]⁺.

Synthesis of compound cis-L-9.3. To a solution of cis-L-9.2 (5.3 g, 17.43 mmol, 1.0 equiv) in DMF (40 mL) at 0° C. was added sodium hydride (60%) (1.046 g, 26.151 mmol, 1.5 equiv) and stirred for 30 min. To the mixture was added iodomethane (3.35 mL, 51.9 mmol, 3.0 equiv) dropwise. The reaction mixture was stirred at room temperature for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford cis-L-9.3 (2.0 g, 36%). MS(ES): m/z 319.23 [M+H]⁺.

Synthesis of compound cis-L-9. A mixture of cis-L-9.3 (2.0 g, 6.28 mmol, 1.0 equiv), 10% palladium on carbon (1.0 g) in methanol (20 mL) was stirred under hydrogen for 3 h. The reaction mixture was filtered through a pad of Celite® and rinsed with methanol. The filtrate was concentrated under reduced pressure to afford cis-L-9 (1.3 g, 93%). MS(ES): m/z 229.18 [M+H]⁺.

### Preparation of Intermediate trans-L-9: trans-tert-butyl methyl(3-methylpiperidin-4-yl)carbamate

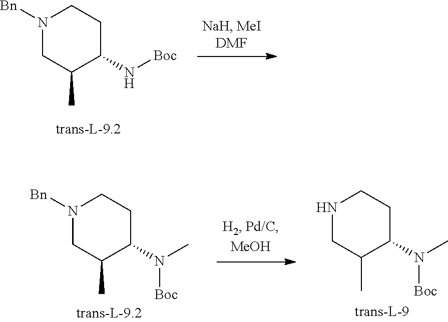 [SMILES: C[C@H]1CN(Cc2ccccc2)CC[C@@H]1NC(=O)OC(C)(C)C ; C[C@H]1CN(Cc2ccccc2)CC[C@@H]1N(C)C(=O)OC(C)(C)C ; CC1CNCC[C@@H]1N(C)C(=O)OC(C)(C)C]

Synthesis of compound trans-L-9. Compound trans-L-9 was prepared from trans-L-9.2 following the procedures described in the synthesis of cis-L-9. MS(ES): m/z 229.34 [M+H].

### Preparation of Intermediate trans-L-10: trans-tert-butyl (3-fluoropiperidin-4-yl)(methyl)carbamate

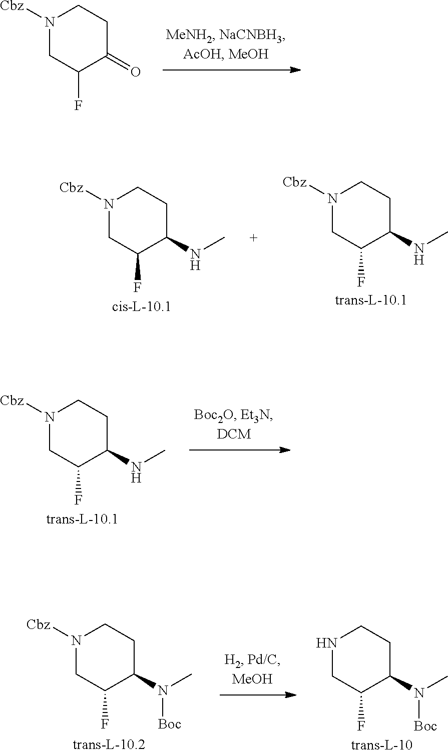 [SMILES: O=C1CCN(C(=O)OCc2ccccc2)CC1F ; CN[C@@H]1CCN(C(=O)OCc2ccccc2)C[C@@H]1F ; CN[C@@H]1CCN(C(=O)OCc2ccccc2)C[C@H]1F ; CN[C@@H]1CCN(C(=O)OCc2ccccc2)C[C@H]1F ; CN(C(=O)OC(C)(C)C)[C@@H]1CCN(C(=O)OCc2ccccc2)C[C@H]1F ; CN(C(=O)OC(C)(C)C)[C@@H]1CCNC[C@H]1F]

Synthesis of compound cis-L-10.1 and trans-L-10.1. To a solution of benzyl 3-fluoro-4-oxopiperidine-1-carboxylate (10 g, 39.84 mmol, 1.0 equiv) in methanol (100 mL) at 0° C. was added dropwise acetic acid (5.0 mL) and 1 M methylamine in methanol (60 mL) followed by sodium cyanoborohydride (3.7 g, 59.76 mmol, 1.5 equiv). The reaction mixture was stirred at room temperature for 3 h. It was quenched with a saturated solution of sodium bicarbonate (100 mL). The reaction mixture was extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.0% methanol in DCM) to afford cis-L-10.1 (3.6 g, 34%), MS(ES: m/z 267.14 [M+H]⁺ and trans-L-10.1 (4.0 g, 38%). MS(ES: m/z 267.14 [M+H]⁺.

Synthesis of compound trans-L-10.2. To a solution of trans-L-10.1 (3.5 g, 13.15 mmol, 1.0 equiv) in DCM (50 mL) at 0° C. was added trimethylamine (3.6 mL, 26.31 mmol, 2.0 equiv) and di-tert-butyl dicarbonate (6.3 mL, 26.3 mmol, 2.0 equiv) dropwise. The reaction mixture was stirred at room temperature for 1 h. It was quenched with water (60 mL) and extracted with DCM (50 mL). The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford trans-L-10.2 (1.5 g, 31%). MS(ES): m/z 367.20 [M+H]⁺.

Synthesis of compound trans-L-10. A mixture of compound trans-L-10.2 (1.5 g, 4.09 mmol, 1.0 equiv), methanol (30 mL) and 10% palladium on carbon (1.0 g) was stirred under hydrogen for 1 h. The reaction mixture was filtered through a pad of Celite® and rinsed with methanol. The filtrate was concentrated under reduced pressure to afford trans-L-10 (1.2 g, 100%). MS(ES): m/z 233.16 [M+H]⁺.

### Preparation of Intermediate cis-L-10: cis-tert-butyl (3-fluoropiperidin-4-yl)(methyl)carbamate

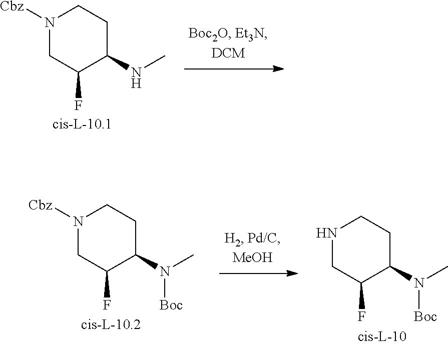 [SMILES: CN[C@@H]1CCN(C(=O)OCc2ccccc2)C[C@@H]1F ; CN(C(=O)OC(C)(C)C)[C@@H]1CCN(C(=O)OCc2ccccc2)C[C@@H]1F ; CN(C(=O)OC(C)(C)C)[C@@H]1CCNC[C@@H]1F]

Synthesis of compound cis-L-10. Compound cis-L-10 was prepared from cis-L-10.1 following the procedures described in the synthesis of trans-L-10. MS(ES): m/z 233.16 [M+H]⁺. Preparation of intermediate trans-L-11: trans-benzyl (3-fluoropiperidin-4-yl)(methyl)carbamate hydrochloride

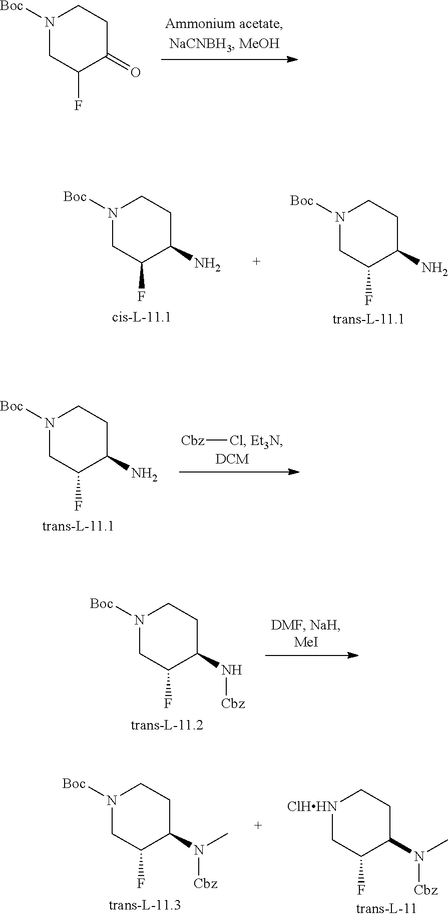

Synthesis of compound cis-L-11.1 and trans-L-11.1. To a solution of tert-butyl 3-fluoro-4-oxopiperidine-1-carboxylate (5.0 g, 23.02 mmol, 1.0 equiv) in methanol (50.0 mL) was added ammonium acetate (12.42 g, 161.11 mmol, 7 equiv) and sodium cyanoborohydride (1.74 g, 27.62 mmol, 1.5 equiv). The reaction mixture was stirred at room temperature for 16 h. It was transferred into water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.6% methanol in DCM) to afford cis-L-11.1 and trans-L-11.1 (0.48 g, 10%). MS(ES): m/z 219.14 [M+H]⁺.

Synthesis of compound trans-L-11.2. To a solution of trans-L-11.1 (0.480 g, 2.20 mmol, 1 equiv) and triethylamine (0.667 g, 6.60 mmol 3.0 equiv) in DCM (5.0 mL) was added benzyl chloroformate (0.450 g, 2.64 mmol, 1.2 equiv). The reaction mixture was stirred at room temperature for 3 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 80% ethyl acetate in hexane) to afford trans-L-11.2 (0.40 g, 52%). m/z: 353.41 [M+H]⁺.

Synthesis of compound trans-L-11.3. To a solution of trans-L-11.2 (0.4 g, 1.14 mmol, 1.0 equiv) in DMF (5.0 mL) at 0° C. was added sodium hydride (60 wt %, 0.091 g, 2.27 mmol, 2.0 equiv). Methyl iodide (0.483 g, 3.41 mmol, 3.0 equiv) was added and the reaction mixture was stirred at room temperature for 2 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 60% ethyl acetate hexane) to afford trans-L-11.3 (0.38 g, 91%). MS(ES): m/z 367.43 [M+H]⁺.

Synthesis of compound trans-L-11. To a solution of trans-L-11.3 (0.38 g, 3.96 mmol, 1.0 equiv) in DCM (4.0 mL) was added 4 M hydrochloric acid in 1,4-dioxane (4.0 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 90% ethyl acetate in hexane) to afford trans-L-11 (0.30 g, 100%). MS(ES): m/z 267.22 [M+H]⁺.

### Preparation of Intermediate (S,S)-L-11: benzyl ((3S,4S)-3-fluoropiperidin-4-yl)(methyl)carbamate

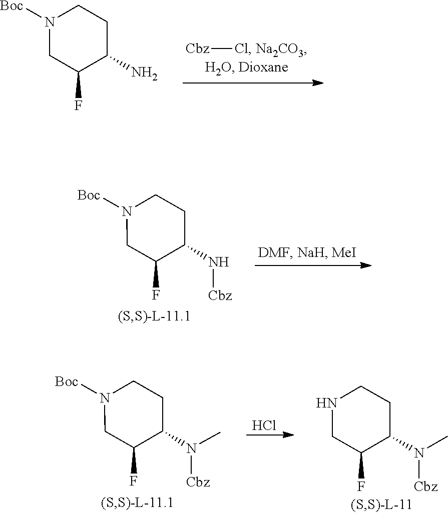

Synthesis of compound (S,S)-L-11.1. To a solution of tert-butyl (3S,4S)-4-amino-3-fluoropiperidine-1-carboxylate (1 g, 4.58 mmol, 1.0 equiv) in dioxane (20 mL) was added saturated sodium carbonate solution in water (10 mL) at 0° C. To the mixture was added benzyl chloroformate (50% solution in toluene, 3.2 mL, 9.17 mmol, 2 equiv) at 0° C. and stirred for 30 min. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.1% methanol in DCM) to afford (S,S)-L-11.1 (1.09 g, 68%). MS(ES): m/z 353.41 [M+H]⁺.

Synthesis of compound (S,S)-L-11.2. To a solution of compound (S,S)-L-11.1 (1.09 g, 3.12 mmol, 1.0 equiv) in DMF (20 mL) was added sodium hydride (0.25 g, 6.2 mmol, 2 equiv) in portions and stirred for 30 min at 0° C. To the mixture was added dropwise methyl iodide (0.886 g, 6.2 mmol, 2 equiv). The reaction mixture was stirred at room temperature for 30 min. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 38% ethyl acetate in hexane) to afford (S,S)-L-11.2 (0.91 g, 79%). MS(ES): m/z 367.28 [M+H]⁺.

Synthesis of compound (S,S)-L-11. To a solution of (S,S)-L-11.2 (0.91 g, 2.45 mmol, 1.0 equiv) in DCM (5 mL) was added hydrochloric acid (4.0 M in dioxane, 5 mL) at 0° C. The reaction mixture was stirred at room temperature for 4 h. It was concentrated under reduced pressure to afford (S,S)-L-11 (0.44 g, 69%). MS(ES): m/z 267.2 [M+H]⁺.

The following intermediates were prepared following the procedure of (S,S)-L-11:

| Intermediate | Name | MS (ES): m/z, |
|---|---|---|
| 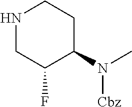 | benzyl ((3R,4R)-3- fluoropiperidin-4- yl)(methyl)carbamate | 367.26 [M + H]⁺ |
| (R,R)-L-11 | | |
| 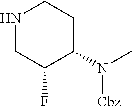 | benzyl ((3R,4S)-3- fluoropiperidin-4- yl)(methyl)carbamate | 367.26 [M + H]⁺ |
| (R,S)-L-11 | | |
| 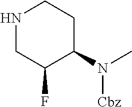 | benzyl ((3S,4R)-3- fluoropiperidin-4- yl)(methyl)carbamate | 367.3 [M + H]⁺ |
| (S,R)-L-11 | | |

### Preparation of Intermediate cis-L-11: cis-benzyl (3-fluoropiperidin-4-yl)(methyl)carbamate hydrochloride

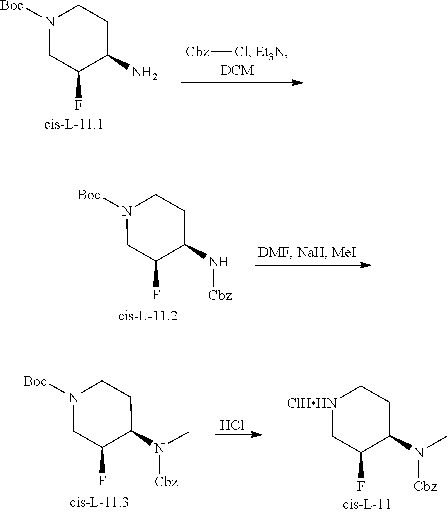

Synthesis of compound cis-L-11. Compound cis-L-11 was prepared from cis-L-11.1 following the procedures described in the synthesis of trans-L-11. MS(ES): m/z 267.12 [M+H]⁺.

### Preparation of Intermediate L-12: benzyl 1,7-diazaspiro[3.5]nonane-1-carboxylate

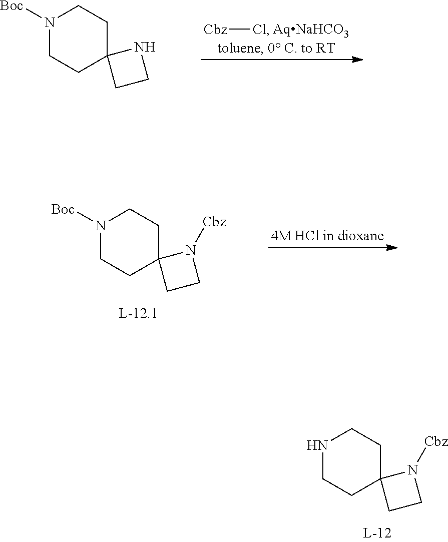 [SMILES: CC(C)(C)OC(=O)N1CCC2(CCN2)CC1 ; O=C(Cl)OCc1ccccc1 ; CC(C)(C)OC(=O)N1CCC2(CC1)CCN2C(=O)OCc1ccccc1 ; O=C(OCc1ccccc1)N1CCC12CCNCC2]

Synthesis of compound L-12.1. To a mixture of tert-butyl 1,7-diazaspiro[3.5]nonane-7-carboxylate (1 g, 4.42 mmol, 1.0 equiv), toluene and aqueous sodium bicarbonate solution (0.743 g, 8.88 mmol, 2.0 equiv in 10 mL water) was added benzyl chloroformate (0.75 g, 4.42 mmol, 1.0 equiv) at 0° C. The reaction mixture was stirred at room temperature for 30 min. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford L-12.1 (1.0 g, 63%). MS(ES): m/z 361.45 [M+H]⁺.

Synthesis of compound L-12. To a solution of L-12.1 (1 g, 2.7 mmol, 1.0 equiv) in DCM (5 mL) at 0° C. was added 4 M hydrochloric acid in dioxane (10 mL) and stirred at room temperature for 1 h. It was concentrated under reduced pressure which was used for next step without further purification L-12 (0.7 g, 97%). MS (ES): m/z 261.34 [M+H]⁺.

### Preparation of Intermediate L-13: methyl 1,8-diazaspiro[4.5]dec-3-ene-1-carboxylate

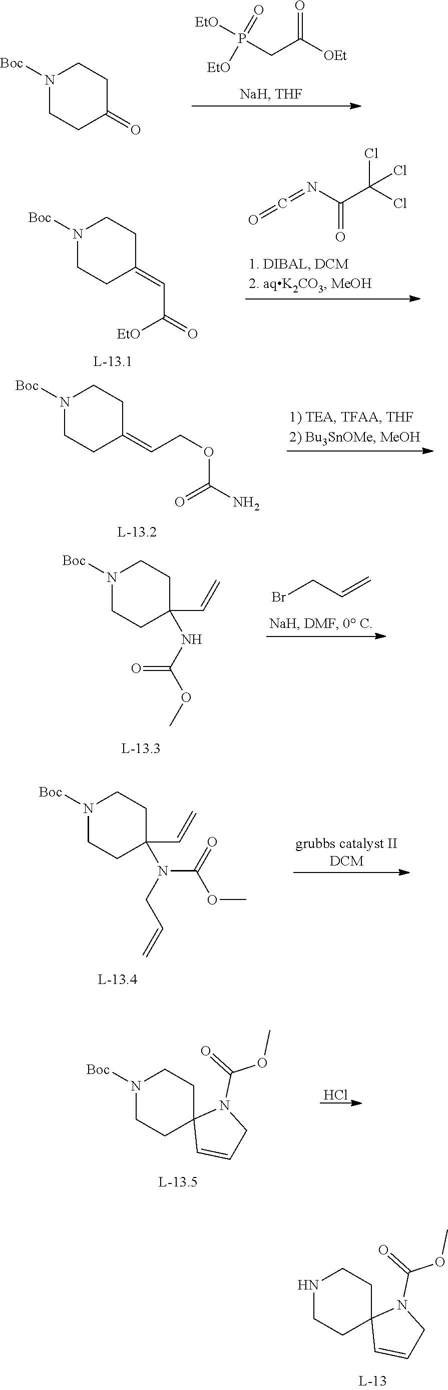

Synthesis of compound L-13.1. To a solution of tert-butyl 4-oxopiperidine-1-carboxylate (50 g, 251 mmol, 1.0 equiv) in THE (500 mL) at 0° C. was added sodium hydride (7.2 g, 502 mmol, 2.0 equiv) in portions and stirred for 30 min. To the mixture was added ethyl 2-(diethoxyphosphoryl)acetate (84 g, 376 mmol, 1.5 equiv). It was stirred at room temperature for 2 h, quenched with saturated solution of ammonium chloride and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford L-13.1. (34.4 g, 50%). MS(ES): m/z 270.16 [M+H]⁺.

Synthesis of compound L-13.2. To a solution of L-13.1 (34 g, 125 mmol, 1.0 equiv) in THE (400 mL) at 0° C. was added dropwise diisobutylaluminium hydride (2 M in THF, 81 mL, 162 mmol, 1.3 equiv) and stirred at 0° C. for 2 h. It was allowed to warm to room temperature and stirred for 30 min. It was quenched with hydrochloric acid (1.5 M) and the mixture was filtered through a pad of Celite®. The filtrate was extracted with DCM. The combined organic layers were washed with brine, dried over anhydrous sodium sulfate, filtered, and concentrated under reduced pressure. The residue was added methanol (340 mL), potassium carbonate (69 g, 500 mmol, 4 equiv) in water and 2,2,2-trichloroacetyl isocyanate (37 mL, 312 mmol, 2.5 equiv) at 0° C. The mixture was stirred for 1 h at room temperature. It was extracted with DCM. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-13.2 (14.3 g, 42%). MS(ES): m/z 271.16 [M+H]⁺.

Synthesis of compound L-13.3. To a solution of L-13.2 (6.0 g, 22.22 mmol, 1.0 equiv) in THE (120 mL) at 0° C. was added triethylamine (81 mL, 162 mmol, 6.0 equiv), stirred for 10 min and added trifluoroacetic anhydride (6.1 mL, 44.44 mmol, 2.0 equiv). The reaction mixture was stirred at room temperature for 1 h. To the mixture was added methanol (60 mL) and tributyltin methoxide (0.927 g, 2.88 mmol, 0.13 equiv), and stirred for 16 h at room temperature. It was quenched with water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-13.3 (2.9 g, 46%). MS(ES): m/z 285.14 [M+H]⁺.

Synthesis of compound L-13.4. To a solution of L-13.3 (2.9 g, 10.17 mmol, 1.0 equiv) in DMF (30 mL) at 0° C. was added sodium hydride (0.219 g, 15.26 mmol, 1.5 equiv) in portions and stirred for 30 min. To the mixture was added allyl bromide (1.8 g, 15.26 mmol, 1.5 equiv). The reaction mixture was stirred at room temperature for 1 h. It was quenched with saturated solution of ammonium chloride and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford L-13.4 (1.4 g, 42%). MS(ES): m/z 325.20 [M+H]⁺.

Synthesis of compound L-13.5. To a solution of L-13.4 (1.4 g, 4.32 mmol, 1.0 equiv) in DCM (15 mL) was added Grubbs catalyst second generation (0.732 g, 0.86 mmol, 0.2 equiv) and stirred at room temperature for 16 h. It was quenched with water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford L-13.5 (0.85 g, 66%). MS(ES): m/z 297.17 [M+H]⁺.

Synthesis of compound L-13. To a solution of L-13.5 (0.850 g, 2.86 mmol, 1.0 equiv) in DCM (10 mL) was added 4.0 M hydrochloric acid in dioxane (2.0 mL). The reaction mixture was stirred at room temperature for 6 h. It was concentrated under reduced pressure. The crude material was diluted with DCM and quenched with saturated solution of bicarbonate and the reaction mixture was extracted with DCM. The combined organic layers were washed with brine, dried over anhydrous sodium sulfate, filtered, and concentrated under reduced pressure. The residue was purified by trituration with diethyl ether to afford L-13 (0.75 g, 100%). MS(ES): m/z 197.12 [M+H]⁺.

### Preparation of Intermediate L-14: tert-butyl (3-azabicyclo[3.2.1]octan-8-yl)(methyl)carbamate

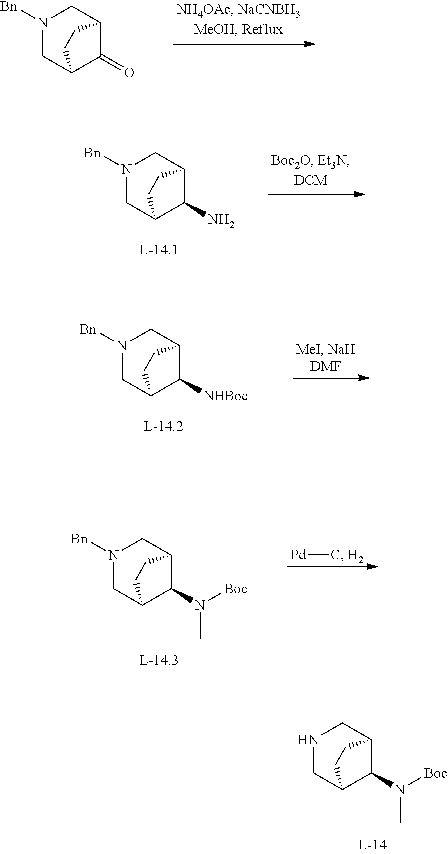

Synthesis of compound L-14.1. To a solution of (1R,5S)-3-benzyl-3-azabicyclo[3.2.1]octan-8-one (5.0 g, 23.25 mmol, 1.0 equiv) in methanol (50 mL) at 0° C. was added ammonium acetate (17.9 g, 232 mmol, 10 equiv). The reaction mixture was stirred at room temperature for 4 h. Sodium cyanoborohydride (1.5 g, 23.25 mmol, 1.0 equiv) was added in portions and the reaction mixture was stirred at 80° C. for 16 h. It was concentrated under reduced pressure. To the residue was added water, adjusted to pH 4 with 1.5 N hydrochloric acid and extracted with chloroform. The aqueous layer was separated and basified (pH 9) using 10% ammonia solution and extracted with chloroform. The combined organic layers after basification were washed with brine, dried over anhydrous sodium sulfate, filtered, and concentrated under reduced pressure to afford L-14.1 (3.0 g, 60%). MS (ES): m/z 217.2 [M+1]⁺.

Synthesis of compound L-14.2. To a solution of L-14.1 (3.0 g, 13.81 mmol, 1.0 equiv) in dichloromethane (30 mL) at 0° C. was added trimethylamine (0.4 mL, 2.76 mmol, 0.2 equiv) and di-tert-butyl dicarbonate (3.0 mL, 13.18 mmol, 1.0 equiv). The reaction mixture was stirred at room temperature for 2 h. It was added to water (30 mL) and extracted with dichloromethane. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford L-14.2 (2.5 g, 57%). MS(ES): m/z 318.28 [M+H]⁺.

Synthesis of compound L-14.3. To a solution of L-14.2 (2.5 g, 7.88 mmol, 1.0 equiv) in DMF (25 mL) at 0° C. was added sodium hydride (0.283 g, 19.71 mmol, 2.5 equiv) in portions and stirred for 30 min. To the mixture was added iodomethane (3.0 mL, 47.28 mmol, 6.0 equiv) dropwise and stirred at room temperature for 1 h. It was transferred into water and extracted with ethyl acetate. The combined organic layers were with brine, dried over anhydrous sodium sulfate, filtered, and concentrated under reduced pressure to afford crude The residue was purified by flash column chromatography on silica gel (CombiFlash®, 10% ethyl acetate in hexane) to afford L-14.3 (2.0 g, 77%). MS(ES): m/z 330.9 [M+H]⁺ and 332.4 [M+H]⁺.

Synthesis of compound L-14. A mixture of compound L-14.3 (2.0 g, 6.28 mmol, 1.0 equiv), methanol (30 mL) and 10% palladium on carbon (0.2 g) was stirred under hydrogen (1 atm) for 3 h. It was filtered through a pad of Celite® and rinsed with methanol. The filtrate was concentrated under reduced pressure to afford L-14 (1.5 g, 99%). MS(ES): m/z 241.24 [M+H]⁺.

### Preparation of Intermediate L-15: benzyl methyl(piperidin-4-yl)carbamate

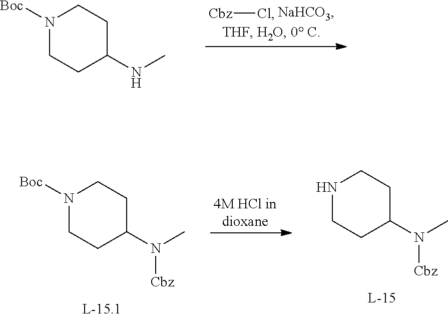 [SMILES: CNC1CCN(C(=O)OC(C)(C)C)CC1 ; O=C(Cl)OCc1ccccc1 ; CN(C(=O)OCc1ccccc1)C1CCN(C(=O)OC(C)(C)C)CC1 ; CN(C(=O)OCc1ccccc1)C1CCNCC1]

Synthesis of compound L-15.1. To a solution of tert-butyl 4-(methylamino)piperidine-1-carboxylate (0.8 g, 3.73 mmol, 1.0 equiv) in THF (10 mL) and water (5.0 mL) was added sodium bicarbonate (0.8 g, 7.47 mmol, 2.0 equiv) at 0° C. Benzyl chloroformate (0.5 mL, 3.73 mmol, 1.0 equiv) was added dropwise and the reaction mixture was stirred at room temperature for 6 h. It was transferred into a saturated solution of sodium bicarbonate (10 mL) and extracted with DCM. The combined organic layers were washed with brine, dried over anhydrous sodium sulfate, filtered, and concentrated under reduced pressure. The residue was purified by flash column chromatography on silica gel (CombiFlash®, 1.5% methanol in DCM) to afford L-15.1 (0.550 g, 42%). MS(ES): m/z: 349.20 [M+H]⁺.

Synthesis of compound L-15. To a solution of L-15.1 (0.550 g, 1.58 mmol, 1.0 equiv) in DCM (5.0 mL) at 0° C. was added 4 M hydrochloric acid in dioxane (0.5 mL). The reaction mixture was stirred at room temperature for 2 h. It was concentrated to afford compound L-15 (0.550 g, 100%). MS(ES): m/z: 249.20 [M+H]⁺.

### Preparation of Intermediate cis-L-16: cis-benzyl (3-methoxypiperidin-4-yl)(methyl)carbamate

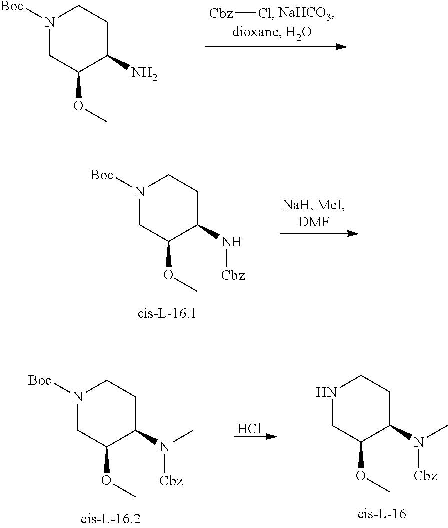 [SMILES: CO[C@H]1CN(C(=O)OC(C)(C)C)CC[C@H]1N ; O=C(Cl)OCc1ccccc1 ; CO[C@H]1CN(C(=O)OC(C)(C)C)CC[C@H]1NC(=O)OCc1ccccc1 ; CO[C@H]1CN(C(=O)OC(C)(C)C)CC[C@H]1N(C)C(=O)OCc1ccccc1 ; Cl ; CO[C@H]1CNCC[C@H]1N(C)C(=O)OCc1ccccc1]

Synthesis of compound cis-L-16.1. To a solution of tert-butyl (3S,4R)-4-amino-3-methoxypiperidine-1-carboxylate (1.0 g, 4.34 mmol, 1.0 equiv) in THF (10 mL) and water (5.0 mL) was added sodium bicarbonate (0.729 g, 8.68 mmol, 2.0 equiv) at 0° C. Benzyl chloroformate (0.6 mL, 3.34 mmol, 1.0 equiv) was added dropwise and the reaction mixture was stirred at room temperature for 2 h. It was transferred into a saturated solution of sodium bicarbonate (10 mL) and extracted with DCM. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford cis-L-16.1 (0.9 g, 57%). MS(ES): m/z: 365.20 [M+H]⁺.

Synthesis of compound cis-L-16.2. To a solution of cis-L-16.1 (0.9 g, 2.47 mmol, 1.0 equiv) in DMF (10 mL) at 0° C. was added sodium hydride (0.072 g, 4.94 mmol, 2.5 equiv) and stirred for 10 min. To the mixture was added iodomethane (0.9 mL, 14.82 mmol, 6.0 equiv) dropwise. The reaction mixture was stirred at room temperature for 1 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford cis-L-16.2 (0.75 g, 79%). MS(ES): m/z 379.22 [M+H]⁺.

Synthesis of compound cis-L-16. To a solution of cis-L-16.2 (0.750 g, 1.97 mmol, 1.0 equiv) in DCM (10 mL) was added 4.0 M hydrochloric acid in dioxane (2.0 mL). The reaction mixture was stirred at room temperature for 6 h. It was concentrated under reduced pressure. The material was diluted with DCM and quenched with saturated solution of bicarbonate and the reaction mixture was extracted with DCM. The combined organic layers were washed with brine, dried over anhydrous sodium sulfate, filtered, and concentrated under reduced pressure. The residue was purified by flash column chromatography on silica gel (40% ethyl acetate in hexane) to afford cis-L-16 (0.40 g, 74%). MS(ES): m/z 279.16 [M+H]⁺.

### Preparation of Intermediate trans-L-16: trans-benzyl (3-methoxypiperidin-4-yl)(methyl)carbamate

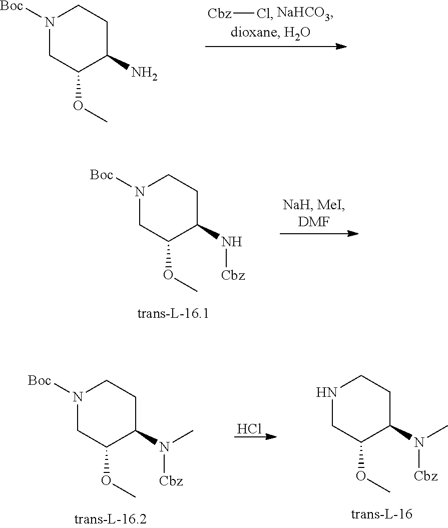 [SMILES: CO[C@@H]1CN(C(=O)OC(C)(C)C)CC[C@H]1N ; O=C(Cl)OCc1ccccc1 ; CO[C@@H]1CN(C(=O)OC(C)(C)C)CC[C@H]1NC(=O)OCc1ccccc1 ; CO[C@@H]1CN(C(=O)OC(C)(C)C)CC[C@H]1N(C)C(=O)OCc1ccccc1 ; Cl ; CO[C@@H]1CNCC[C@H]1N(C)C(=O)OCc1ccccc1]

Synthesis of compound trans-L-16. trans-L-16 was prepared following the procedures described in the synthesis of cis-L-16. MS(ES): m/z 279.16 [M+H]⁺.

### Preparation of Intermediate L-17: benzyl (3,3-difluoropiperidin-4-yl)(methyl)carbamate

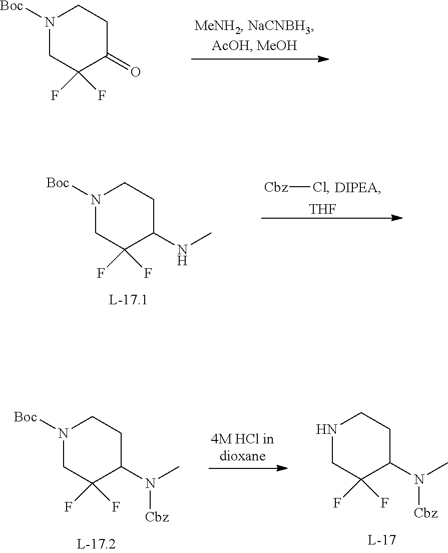 [SMILES: CC(C)(C)OC(=O)N1CCC(=O)C(F)(F)C1 ; CNC1CCN(C(=O)OC(C)(C)C)CC1(F)F ; O=C(Cl)OCc1ccccc1 ; CN(C(=O)OCc1ccccc1)C1CCN(C(=O)OC(C)(C)C)CC1(F)F ; CN(C(=O)OCc1ccccc1)C1CCNCC1(F)F ; "THF"]

Synthesis of compound L-17.1 To a solution of tert-butyl 3,3-difluoro-4-oxopiperidine-1-carboxylate (10 g, 44.05 mmol, 1.0 equiv), acetic acid (5.0 mL) and 1 M methylamine in methanol (60 mL) in methanol (100 mL) at 0° C. was added sodium cyanoborohydride (4.0 g, 66.07 mmol, 1.5 equiv). The reaction mixture was stirred at room temperature for 3 h. It was quenched with a saturated solution of sodium bicarbonate (100 mL). The reaction mixture was extracted with ethyl acetate. The combined organic layers were washed with brine, dried over anhydrous sodium sulfate, filtered, and concentrated under reduced pressure. The residue was purified by flash column chromatography on silica gel (CombiFlash®, 5.0% methanol in DCM in 7.0% ammonia in methanol) to afford L-17.1 (3.5 g, 32%). MS(ES: m/z 251.29 [M+H]⁺.

Synthesis of compound L-17.2 To a solution of L-17.1 (2.0 g, 7.96 mmol, 1.0 equiv) in THE (30 mL) was added N,N-diisopropylethylamine (2.4 mL, 19.92 mmol, 2.5 equiv) and benzylchloroformate (1.3 mL, 9.55 mmol, 1.2 equiv). The reaction mixture was stirred at room temperature for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate hexane) to afford L-17.2 (2.0 g, 74.07%). MS(ES): m/z 385.19 [M+H]⁺.

Synthesis of compound L-17. To a solution of L-17.2 (2.0 g, 5.73 mmol, 1.0 equiv) in 1,4-dioxane (20 mL) was added 4 M hydrochloric acid in 1,4-dioxane (2.0 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure to afford L-17 (1.3 g, 80%). MS(ES): m/z 285.13 [M+H]⁺.

### Preparation of Intermediate cis-L-18: cis-benzyl methyl(2-methylpiperidin-4-yl)carbamate

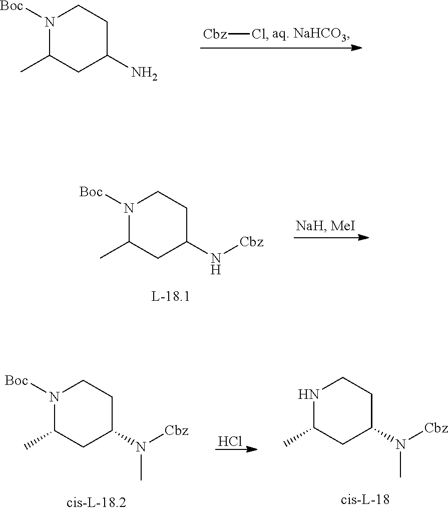 [SMILES: CC1CC(N)CCN1C(=O)OC(C)(C)C ; O=C(Cl)OCc1ccccc1 ; CC1CC(NC(=O)OCc2ccccc2)CCN1C(=O)OC(C)(C)C ; C[C@H]1C[C@@H](N(C)C(=O)OCc2ccccc2)CCN1C(=O)OC(C)(C)C ; Cl ; C[C@H]1C[C@@H](N(C)C(=O)OCc2ccccc2)CCN1]

Synthesis of compound L-18.1. To a solution of tert-butyl 4-amino-2-methylpiperidine-1-carboxylate (3.0 g, 14.00 mmol, 1.0 equiv) in THE (20 mL) was added sodium bicarbonate (3.53 g, 42.00 mmol, 3.0 equiv) at 0° C. followed by the addition of benzylchloroformate (3.58 g, 21.00 mmol, 1.5 equiv). The reaction mixture was stirred at room temperature for 6 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (20% ethyl acetate in hexane) to afford L-18.1 (3.2 g, 66%). MS(ES): m/z 349.20 [M+H]⁺.

Synthesis of compound cis-L-18.2. To a solution of L-18.1 (3.2 g, 9.18 mmol, 1.0 equiv) in DMF (20 mL) at 0° C. was added sodium hydride (0.53 g, 13.793 mmol, 1.5 equiv) and stirred for 15 min. To the mixture was added methyl iodide (1.96 g, 13.78 mmol, 1.5 equiv) and stirred at room temperature for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (10% ethyl acetate in hexane) to afford cis-L-18.2 (2.8 g, 84%). MS(ES): m/z 363.22 [M+H]⁺.

Synthesis of compound cis-L-18. To a solution of cis-L-18.2 (2.8 g, 7.72 mmol, 1.0 equiv) in DCM (5 mL) was added 4.0 M hydrochloric acid in dioxane (2.8 mL) at 0° C. The reaction mixture was stirred at room temperature for 3 h. It was concentrated under vacuum to afford cis-L-18 (2.1 g, 91%). MS(ES): m/z 262.14 [M+H]⁺.

### Preparation of Intermediate trans-L-19: trans-tert-butyl ((3R,4R)-3-methoxypiperidin-4-yl)(methyl)carbamate

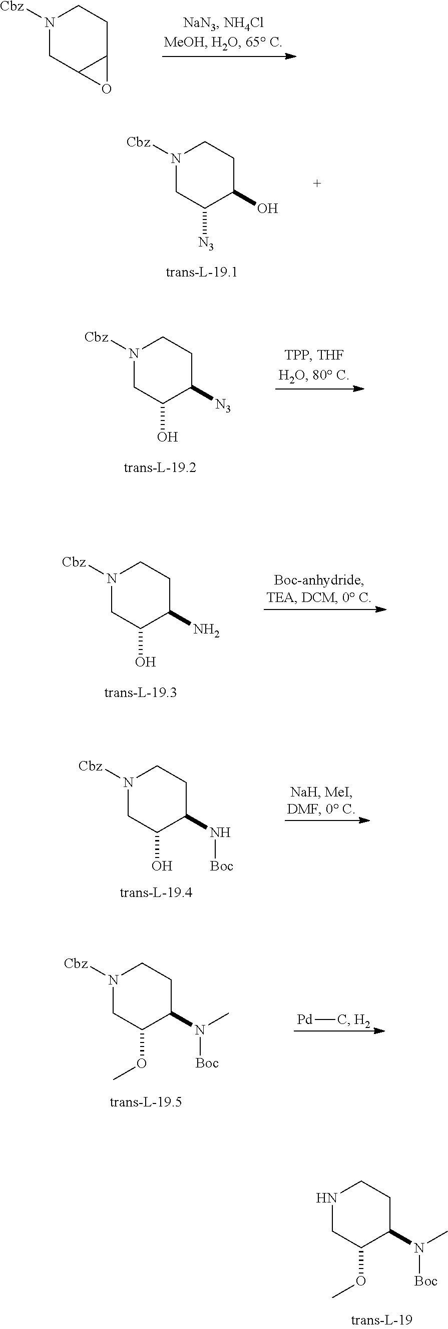

Synthesis of compound trans-L-19.1 and trans-L-19.2. A solution of benzyl 7-oxa-3-azabicyclo[4.1.0]heptane-3-carboxylate (5.0 g, 21.43 mmol, 1.0 equiv) in methanol (60 mL) and water (12 mL), sodium azide (2.09 g, 32.15 mmol, 1.5 equiv) and ammonium chloride (1.15 g, 21.43 mmol, 1.5 equiv) was stirred at 60° C. for 2 h. It was poured over water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford trans-L-19.2 (3.0 g, 51%). MS(ES): m/z 277.30 [M+H]⁺.

Synthesis of compound trans-L-19.3. To a solution of trans-L-19.2 (3.0 g, 10.86 mmol, 1 equiv) in THE (50.0 mL) and water (10 mL) was added triphenylphosphine (5.7 g, 21.72 mmol, 2.0 equiv) and stirred at 80° C. for 3 h. It was quenched with water (10 mL) and extracted with ethyl acetate. The combined organic layers were washed with brine and dried over anhydrous sodium sulfate, filtered and concentrated under reduced pressure. The residue was purified by flash column chromatography on silica gel (CombiFlash®, 12% ethyl acetate in hexane) to afford trans-L-19.3 (2.2 g, 81%). MS(ES): m/z: 251.30 [M+H]⁺.

Synthesis of compound trans-L-19.4. To a solution of trans-L-19.3 (2.2 g, 8.79 mmol, 1.0 equiv) and triethylamine (2.67 g, 2.37 mmol, 3 equiv) in DCM (31 mL) was added at 0° C. di-tert-butyl dicarbonate (2.3 g, 10.56 mmol, 1.2 equiv) and stirred for 4 h. It was concentrated under reduced pressure. The residue was dissolved in DCM and washed with a sat. aqueous sodium bicarbonate solution. The organic layer was isolated, washed with brine, dried over anhydrous sodium sulfate, filtered, and concentrated under reduced pressure. The residue was purified by flash column chromatography on silica gel (CombiFlash®, 20% ethyl acetate in hexane) to afford trans-L-19.4 (2.1 g, 68%). MS(ES): m/z 351.18 [M+H]⁺.

Synthesis of compound trans-L-19.5. To a solution of trans-L-19.4 (2.1 g, 5.99 mmol, 1.0 equiv) in DMF (30 mL) at 0° C. was added sodium hydride (60 wt %, 0.2874 g, 0.287 mmol, 1.2 equiv). The mixture was stirred for 15 min. Methyl iodide (1.02 g, 0.79 mmol, 1.2 equiv) was added and the reaction mixture was stirred at room temperature for 2 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate hexane) to afford trans-L-19.5 (1.3 g, 54%). MS(ES): m/z 379.22 [M+H]⁺.

Synthesis of compound trans-L-19. A mixture of trans-L-19.5 (1.3 g, 3.43 mmol, 1.0 equiv), methanol (20 mL) and 10% palladium on carbon (0.5 g) was stirred under hydrogen (1 atm) at 50° C. for 12 h. It was filtered through a pad of Celite® and concentrated under reduced pressure to afford trans-L-19 (0.80 g, 89%). MS(ES): m/z 245.18 [M+H]⁺.

### Preparation of Intermediate trans-L-20: benzyl (4aS,8aS)-octahydro-1H-pyrido[3,4-b][1,4]oxazine-1-carboxylate

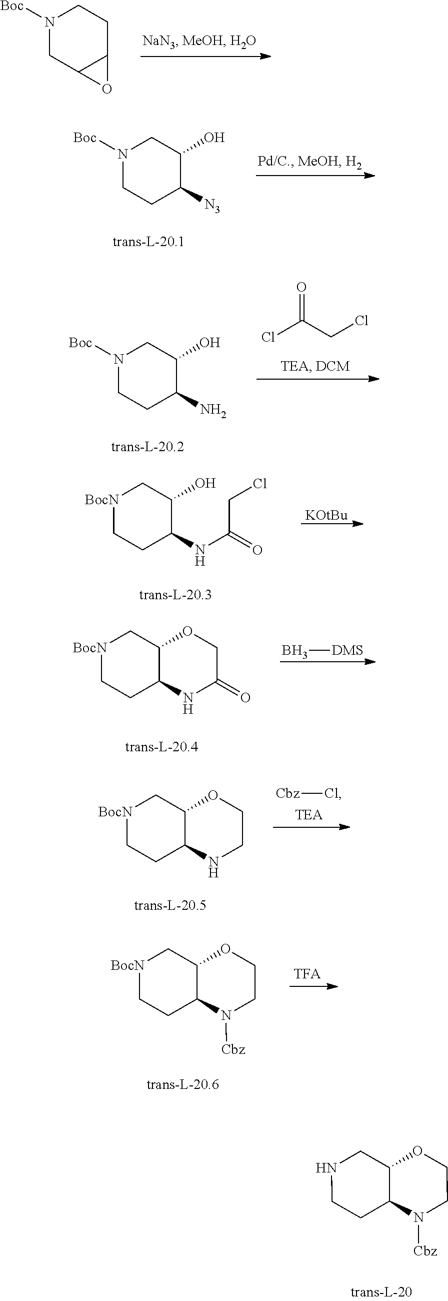

Synthesis of compound trans-L-20.1. To solution of tert-butyl 7-oxa-3-azabicyclo[4.1.0]heptane-3-carboxylate (10.0 g, 42.9 mmol, 1.0 equiv) in methanol (80 mL) and water (20 mL) was added sodium azide (4.18 g, 64.3 mmol, 1.5 equiv). The reaction mixture was stirred for 16 h at 65° C. It was transferred into ice-water and extracted with ethyl acetate. The combined organic layers were washed with sodium bicarbonate solution and brine, dried over sodium sulfate. The residue was purified by flash column chromatography on silica gel (CombiFlash®, 9% ethyl acetate in hexane) to afford trans-L-20.1 (9.0 g, 74%) MS(ES): m/z 243.14 [M+H]⁺.

Synthesis of compound trans-L-20.2. A mixture of trans-L-20.1 (9.0 g, 37.19 mmol, 1 equiv), methanol (90 mL) and 10% palladium on carbon (0.50 g) was stirred under hydrogen. The reaction mixture was filtered through a pad of Celite® and washed with 10% methanol in DCM solution. The filtrate was concentrated under reduced pressure to afford trans-L-20.2 (6.0 g, 75%) MS(ES): m/z 217.15 [M+H]⁺.

Synthesis of compound trans-L-20.3. To a solution of tert-butyl (3S,4S)-4-amino-3-hydroxypiperidine-1-carboxylate (5.0 g, 23.12 mmol) and triethylamine (7.0 g, 69.44 mmol, 3 equiv) in DCM (50 mL) was added 2-chloroacetyl chloride (2.87 g, 25.43 mmol, 1 equiv) at 0° C. The reaction mixture was stirred for 20 min. It was poured into water and the aqueous layer was extracted with DCM. The combined organic layers were dried over sodium sulfate, filtered, and concentrated under pressure to afford trans-L-20.3 (3.4 g, 59%). MS (ES): m/z 293.12 [M+H]⁺.

Synthesis of compound trans-L-20.4. To a stirred solution of trans-L-20.3 (3.4 g, 11.61 mmol, 1.0 equiv) in THE (35 mL) was added potassium tert-butoxide (14.5 mL, 29.03 mmol, 2.5 e g) at 0° C. The mixture was stirred at room temperature for 12 h. It was quenched with saturated solution of ammonium chloride at 0° C. and extracted with ethyl acetate. The organic layer was dried over sodium sulfate and filtered. The filtrate was concentrated in reduced pressure. The residue was purified by flash column chromatography on silica gel (CombiFlash®, 30% ethyl acetate in hexane) to afford trans-L-20.4 (2.3 g, 77%). MS (ES): m/z 257.2 [M+H]⁺.

Synthesis of compound trans-L-20.5. To a stirred solution of trans-L-20.4 (2.3 g, 8.97 mmol, 1.0 equiv) in dry THE (25 mL) was added dropwise borane dimethyl sulfide complex (26.9 mL, 26.92 mmol, 3.0 equiv) at 0° C. The reaction mixture was stirred at room temperature for 12 h. After completion of reaction, methanol (10 mL) was added at room temperature and stirred at reflux for 1 h. The mixture was added to saturated solution of sodium bicarbonate and the aqueous was extracted with ethyl acetate. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford trans-L-20.5 (1.1 g, 51%). MS (ES): m/z 243.2 [M+H]⁺.

Synthesis of compound trans-L-20.6. To a solution of trans-L-20.5 (1.1 g, 4.54 mmol, 1.0 equiv) in DCM (10 mL) was added trimethylamine (0.688 g, 6.8 mmol, 1.5 equiv) and benzyl chloroformate (2.3 g, 6.8 mmol, 1.5 equiv) at 0° C. The reaction mixture was stirred at room temperature for 2 h. It was transferred into water and extracted with DCM. The combined organic layers were washed with brine, dried over anhydrous sodium sulfate, filtered, and concentrated under reduced pressure. The residue was purified by flash column chromatography on silica gel (10% ethyl acetate in hexane) to afford trans-L-20.6 (1.0 g, 59%). MS(ES): m/z 377.20 [M+H]⁺.

Synthesis of compound trans-L-20. To a solution of trans-L-20.6 (1.0 g, 2.66 mmol, 1.0 equiv) in DCM (5 mL) was added trifluoroacetic acid (0.7 mL) at 0° C. The reaction mixture was stirred at room temperature for 3 h. It was concentrated under reduced pressure to afford trans-L-20 (0.423 g, 58%). MS(ES): m/z 277.3 [M+H]⁺.

### Preparation of Intermediate trans-L-21: trans-benzyl (3-methoxypiperidin-4-yl-2,2,6,6-d₄)(methyl)carbamate

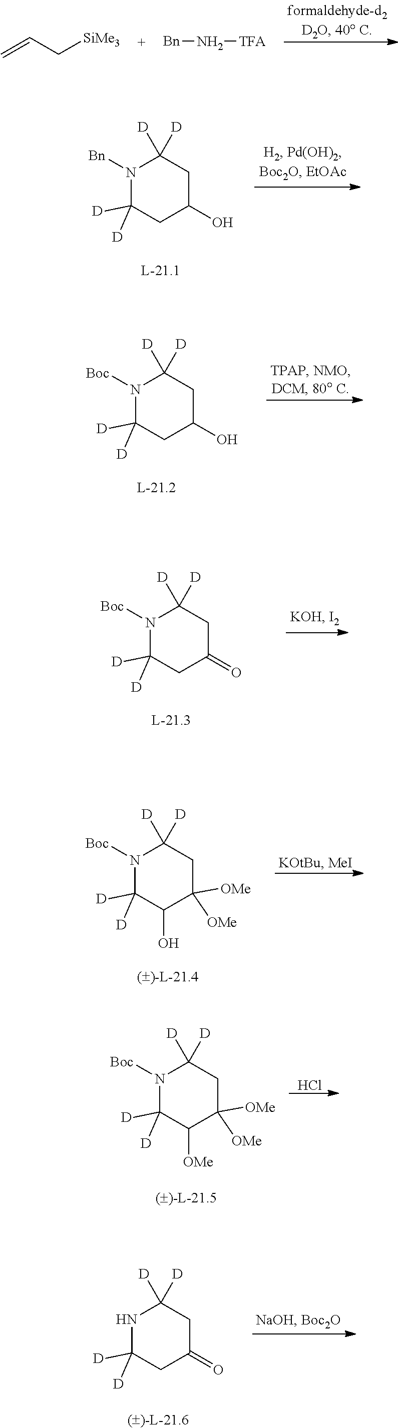

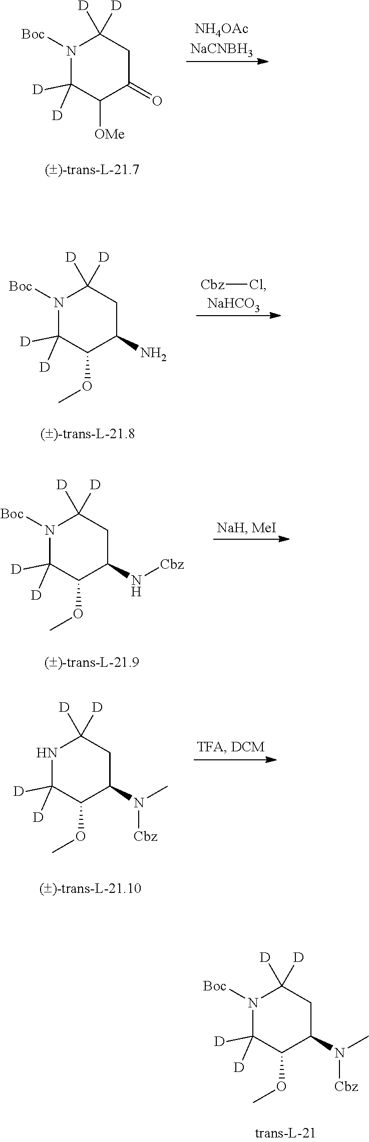

Synthesis of compound L-21.1. To a solution of benzylamine TFA salt (20.0 g, 97.56 mmol, 1.0 equiv) in deuterated water (200 mL) at 0° C. was added deuterated formaldehyde (12.48 g, 390.40 mmol, 4.0 equiv) and stirred for 15 min. To the mixture was added allyltrimethylsilane (12.23 g, 107.31 mmol, 1.1 equiv) and stirred at 40° C. for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (3.1% methanol in DCM) to afford L-21.1 (8.90 g, 47%). MS(ES): m/z 196.30 [M+H]⁺.

Synthesis of compound L-21.2. A mixture of L-21.1 (8.9 g, 45.64 mmol, 1.0 equiv), ethyl acetate (90 mL), 20% palladium hydroxide on carbon (50% of water, 4.5 g) and di-tert-butyl carbonate (14.92 g, 68.46 mmol, 1.5 equiv) was stirred under hydrogen pressure for 16 h. The reaction mixture was filtered through a pad of Celite® and rinsed with methanol. The filtrate was concentrated under reduced pressure. The residue was purified by flash column chromatography on silica gel (CombiFlash®, 2.5% methanol in DCM) to afford L-21.2 (4.3 g, 46%). MS(ES): m/z 206.29 [M+H]⁺.

Synthesis of compound L-21.3. To a solution of L-21.2 (4.3 g, 20.97 mmol, 1.0 equiv) in DCM (43 mL) was added N-methylmorpholine N-oxide (3.68 g, 31.46 mmol, 1.5 equiv) and tetrapropylammonium perruthenate (0.368 g, 1.048 mmol, 0.05 equiv) and stirred at 80° C. for 6 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 60% ethyl acetate in hexane) to afford L-21.3 (3.6 g, 85%). MS(ES): m/z 204.27 [M+H]⁺.

Synthesis of compound (+)-L-21.4. To a solution of L-21.3 (3.6 g, 17.73 mmol, 1.0 equiv) in methanol (36 mL) was added potassium hydroxide (1.78 g, 31.92 mmol, 1.8 equiv) followed by iodine (4.9 g, 19.50 mmol, 1.1 equiv) at 0° C. The reaction mixture was stirred at room temperature for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 60% ethyl acetate in hexane) to afford (+)-L-21.4 (2.9 g, 62%). MS(ES): m/z 266.34 [M+H]⁺.

Synthesis of compound (+)-L-21.5. To a solution of (+)-L-21.4 (2.9 g, 10.94 mmol, 1.0 equiv) in THE (29 mL) was added potassium tert-butoxide (3.06 g, 27.35 mmol, 2.5 equiv) at 0° C. and stirred for 15 min. To the mixture was added methyl iodide (2.33 g, 16.41 mmol, 1.5 equiv) and stirred at room temperature for 16 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (50% ethyl acetate in hexane) to afford (+)-L-21.5 (1.7 g, 56%). MS(ES): m/z 280.37 [M+H]⁺.

Synthesis of compound (+)-L-21.6. To a stirred solution of (+)-L-21.5 (1.7 g, 0.161 mmol, 1 equiv) in 4 M hydrochloric acid in dioxane (17 mL) at 0° C. The reaction mixture was stirred at 50° C. for 2 h. It was poured into a saturated sodium bicarbonate solution extracted with DCM. The combined organic layers were washed with brine, dried over anhydrous sodium sulfate, filtered, and concentrated under reduced pressure to afford (+)-L-21.6 (0.8 g, 99%). MS(ES): m/z 134.18 [M+H]⁺.

Synthesis of compound (+)-L-21.7. To a solution of (+)-L-21.6 (0.8 g, 6.01 mmol, 1.0 equiv) in dioxane (6 mL) was added sodium hydroxide solution in water (0.481 g, 12.03 mmol, 2.0 equiv) followed by di-tert-butyl carbonate (1.44 g, 6.61 mmol, 1.1 equiv) at 0° C. The reaction mixture was stirred at room temperature for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 60% ethyl acetate in hexane) to afford (+)-L-21.7 (1.1 g, 78%). MS(ES): m/z 234.30 [M+H]⁺.

Synthesis of compound (+)-trans-L-21.8. To a solution of (+)-L-21.7 (1.1 g, 4.72 mmol, 1.0 equiv) in methanol (11 mL) was added ammonium acetate (0.436 g, 5.66 mmol, 1.2 equiv) and the reaction mixture was stirred at room temperature for 10 min. To the mixture was added sodium triacetoxyborohydride (2.0 g, 9.44 mmol, 2.0 equiv) and stirred at room temperature for 1 h. It was transferred into ice-water, stirred, and extracted with ethyl acetate. The combined organic layers were washed with brine, dried over anhydrous sodium sulfate, filtered, and concentrated under reduced pressure. The residue was purified by flash column chromatography on silica gel (2.5-3.0% methanol in DCM) to afford (+)-trans-L-21.8 (0.515 g, 50%). MS (ES): m/z 235.33 [M+H]⁺.

Synthesis of compound (+)-trans-L-21.9. To a solution of (+)-trans-L-21.8 (0.515 g, 2.20 mmol, 1.0 equiv) in dioxane (4 mL) and water (2 mL) was added sodium bicarbonate (0.554 g, 6.6 mmol, 3.0 equiv) at 0° C. Benzyl chloroformate (0.376 g, 2.20 mmol, 1.0 equiv) was added dropwise and the reaction mixture was stirred at room temperature for 1 h. It was added into water and extracted with DCM. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford (+)-trans-L-21.9 (0.380 g, 47%). MS(ES): m/z: 369.47 [M+H]⁺.

Synthesis of compound (+)-trans-L-21.10. To a solution of (+)-trans-L-21.9 (0.380 g, 1.03 mmol, 1.0 equiv) in DMF (5 mL) was added sodium hydride (60 wt %, 062 g, 1.54 mmol, 1.5 equiv) in portions and stirred for 15 min at 0° C. To the mixture was added methyl iodide (0.293 g, 2.06 mmol, 2.0 equiv). The reaction mixture was stirred at room temperature for 2 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford (+)-trans-L-21.10 (0.310 g, 79%). MS(ES): m/z 383.49 [M+H]⁺.

Synthesis of compound trans-L-21. To a solution of (+)-trans-L-21.10 (0.310 g, 0.81 mmol, 1.0 equiv) in DCM (2 mL) was added trifluroacetic acid (0.6 mL). The reaction mixture was stirred at room temperature for 1 h. It was transferred into a saturated sodium bicarbonate solution and extracted with DCM. The combined organic layers were washed with brine, dried over anhydrous sodium sulfate, filtered, and concentrated under reduced pressure to afford trans-L-21 (0.210 g, 92%). MS(ES): m/z 283.38 [M+H]⁺.

### Preparation of Intermediate trans-L-22: trans-benzyl ((3S,4S)-3-hydroxypiperidin-4-yl)carbamate

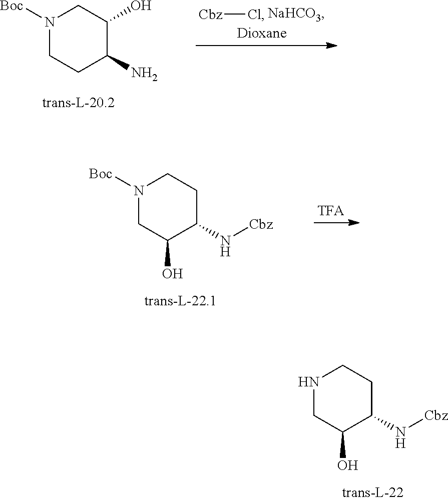 [SMILES: CC(C)(C)OC(=O)N1CC[C@H](N)[C@@H](O)C1 ; O=C(Cl)OCc1ccccc1 ; CC(C)(C)OC(=O)N1CC[C@H](NC(=O)OCc2ccccc2)[C@@H](O)C1 ; O=C(N[C@H]1CCNC[C@@H]1O)OCc1ccccc1]

Synthesis of compound trans-L-22.1. To a solution of trans-L-20.2 (6.0 g, 27.77 mmol, 1.0 equiv) in dioxane (120 mL) at 0° C. was added saturated sodium bicarbonate solution in water (60 mL) at 0° C. The reaction mixture was stirred for 10 min at 0° C. followed by addition of benzyl chloroformate (50% solution in toluene). The reaction mixture was stirred for 30 min at 0° C. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford trans-L-22.1 (4.6 g, 50%). MS(ES): m/z 351.41 [M+H]⁺.

Synthesis of compound trans-L-22. To a solution of trans-L-22.1 (4.6 g, 13.14 mmol, 1.0 equiv) in DCM (3 mL) at 0° C. was added trifluoroacetic acid (3 mL). The reaction mixture was stirred at room temperature for 3 h. It was transferred into a saturated sodium bicarbonate solution and extracted with ethyl acetate. The combined organic layers were washed with a saturated solution of sodium bicarbonate and brine, dried over anhydrous sodium sulfate, filtered, and concentrated under reduced pressure to afford trans-L-22 (2.6 g, 79%). MS(ES): m/z 251.30 [M+H]⁺.

### Preparation of Intermediate T.1: 3-amino-1-methyl-5-(trifluoromethyl)pyridin-2(1H)-one

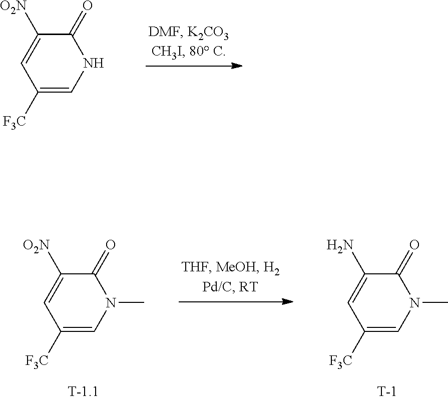

Synthesis of compound T.1. A mixture of 3-nitro-5-(trifluoromethyl)pyridin-2(1H)-one (1.0 g, 4.81 mmol, 1.0 equiv) and potassium carbonate (1.3 g, 9.62 mmol, 2.0 equiv) in DMF (15 mL) was stirred for 15 min before the addition of methyl iodide (1.0 g, 7.21 mmol, 1.5 equiv). The reaction mixture was stirred at 70° C. for 2 h. It was transferred into ice-water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford T.1. MS(ES): m/z 223.12 [M+H]⁺.

Synthesis of compound T-1. A mixture of compound T.1 (0.57 g, 2.57 mmol, 1.0 equiv) and 10% palladium on carbon (0.3 g) in methanol (18 mL) was stirred under hydrogen (1 atm) for 1 h. It was filtered through a pad of Celite® and rinsed with methanol. The filtrate was concentrated under reduced pressure to afford T-1. MS(ES): m/z 193.14 [M+H]⁺.

### Preparation of Intermediate T-2: 3-((4-methylpiperazin-1-yl)methyl)-5-(trifluoromethyl)aniline

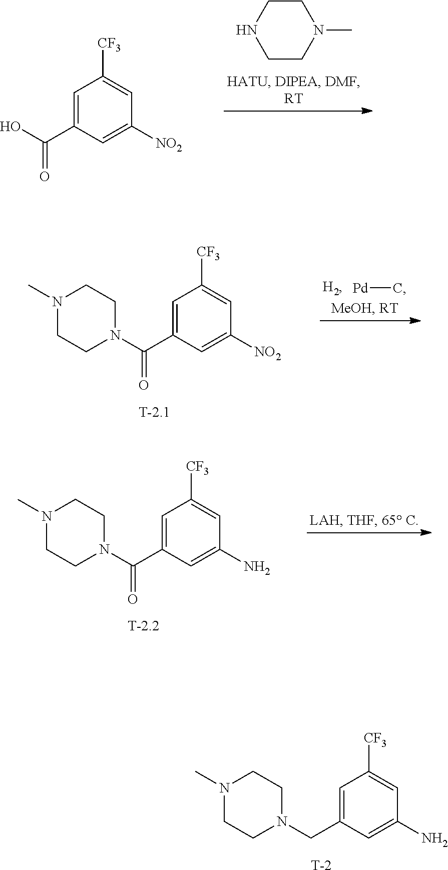

Synthesis of compound T-2.1. A solution of 3-nitro-5-(trifluoromethyl)benzoic acid (2.0 g, 8.51 mmol, 1.0 equiv) and HATU (4.85 g, 12.76 mmol, 1.5 equiv) in DMF (20 mL) was stirred at room temperature for 1 h. To the solution was added 1-methylpiperazine (0.852 g, 8.51 mmol, 1.0 equiv) and N,N-diisopropylethylamine (3.29 g, 25.53 mmol, 3.0 equiv) and stirred for 2 h. It was transferred into water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford T-2.1 (1.6 g, 59%). MS(ES): m/z 318.27 [M+H]⁺.

Synthesis of compound T-2.2. A mixture of compound T-2.1 (1.6 g, 5.04 mmol, 1.0 equiv) and 10% palladium on carbon (0.9 g) in methanol (20 mL) was stirred under hydrogen (1 atm) at room temperature for 2 h. It was filtered through a pad of Celite® and rinsed with methanol. The filtrate was concentrated under reduced pressure. The residue was purified by flash column chromatography on silica gel (CombiFlash®, 5% methanol in DCM) to afford T-2.2 (1.2 g, 83%). MS(ES): m/z 288.29 [M+H]⁺.

Synthesis of compound T-2. To a solution of T-2.2 (1.2 g, 4.18 mmol, 1.0 equiv) in THE (15 mL) was added a solution of lithium aluminum hydride (1 M in THF, 12.54 mL, 12.54 mmol, 3.0 equiv) at 0° C. The reaction mixture was stirred at 65° C. for 3 h. It was cooled to room temperature, transferred into ice-water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.2% methanol in DCM) to afford T-2 (0.620 g, 54%). MS(ES): m/z 274.3 [M+H]⁺.

### Preparation of Intermediate T-3: 1-(3-amino-5-(trifluoromethyl)phenyl)piperidin-4-ol

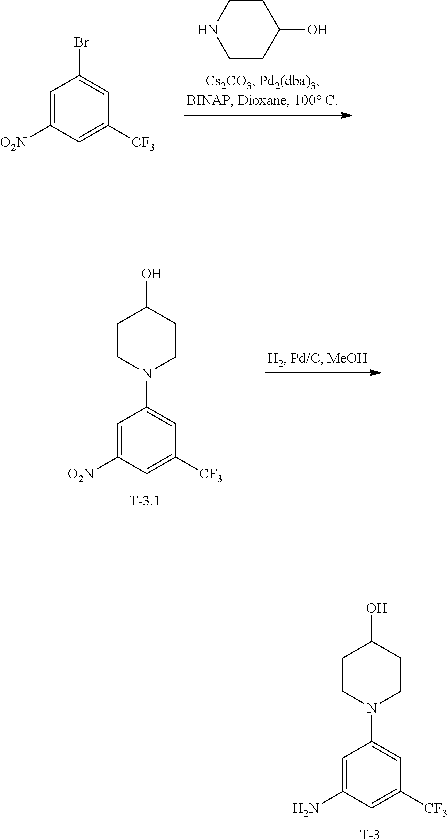

Synthesis of compound T-3.1. A mixture of 1-bromo-3-nitro-5-(trifluoromethyl)benzene (1.0 g, 3.70 mmol, 1.0 equiv), piperidin-4-ol (0.748 g, 7.40 mmol, 2.0 equiv) and cesium carbonate (3.01 g, 9.25 mmol, 2.5 equiv) in 1,4-dioxane (20 mL) was degassed by bubbling through a stream of argon under argon atmosphere for 10 min. 2,2′-Bis(diphenylphosphino)1,1′-binaphthyl (0.230 g, 0.740 mmol, 0.2 equiv) and tris(dibenzylideneacetone)dipalladium (O) (0.338 g, 0.370 mmol, 0.1 equiv) was added and degassed for another 5 min. The reaction mixture was stirred at 110° C. for 5 h. It was cooled to room temperature, filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10-15% ethyl acetate in hexane) to afford T-3.1 (0.500, 46.5%). MS(ES): m/z 291.16 [M+H]⁺.

Synthesis of compound T-3. A mixture compound T-3.1 (0.500 g, 1.72 mmol, 1.0 equiv) and 10% palladium on carbon (0.2 g) in methanol (5 mL) was stirred under hydrogen (1 atm) for 2 h. It was filtered through a pad of Celite® and rinsed with methanol. The filtrate was concentrated under reduced pressure. The residue was purified by flash column chromatography on silica gel (CombiFlash®, 5% methanol in DCM as eluent to afford T-3 (0.350 g, 78%). MS(ES): m/z 260.6 [M+H]⁺.

### Preparation of Intermediate T-4: 6-methyl-4-(trifluoromethyl)pyridin-2-amine

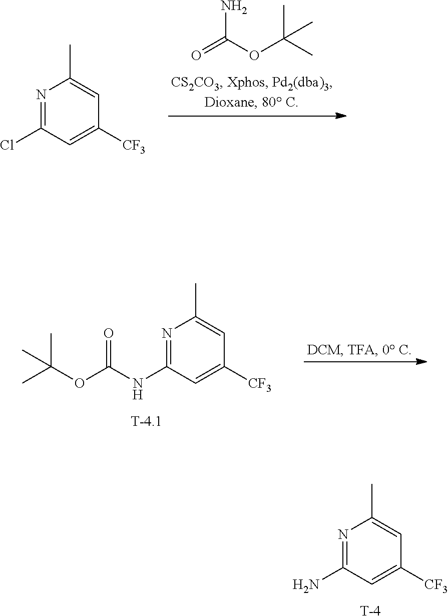

Synthesis of compound T-4.1. A mixture of 2-chloro-6-methyl-4-(trifluoromethyl)pyridine (1.0 g, 5.12 mmol, 1.0 equiv), tert-butyl carbamate (1.2 g, 10.25 mmol, 2.0 equiv) and cesium carbonate (5.8 g, 17.94 mmol, 3.5 equiv) in 1,4-dioxane (10 mL) was degassed by bubbling through a stream of argon for 10 min. Xphos (0.245 g, 0.512 mmol, 0.1 equiv) and tris(dibenzylideneacetone)dipalladium(O) (0.234 g, 0.256 mmol, 0.05 equiv) were added and degassed for another 5 min. The reaction mixture stirred at 80° C. for 2 h. It was cooled to room temperature, transferred into water, and extracted with ethyl acetate. The combined organic layers were washed with brine, dried over anhydrous sodium sulfate, filtered, and concentrated under reduced pressure to afford. The residue was purified by flash column chromatography on silica gel (CombiFlash®, 3.8% methanol in DCM) to afford T-4.1 (0.600 g, 42.5%). MS(ES): m/z: 277.1 [M+H]⁺.

Synthesis of compound T-4. To solution of T-4.1 (0.600 g, 2.895 mmol, 1.0 equiv) in DCM (6 mL) was added trifluoroacetic acid (6.0 mL) at 0° C. and stirred for 1 h. It was transferred into ice-cold saturated sodium bicarbonate solution and extracted with DCM. The combined organic layers were washed with brine, dried over anhydrous sodium sulfate, filtered, and concentrated under reduced pressure to afford T-4 (0.250 g, 65%). MS(ES): m/z: 177.2 [M+H]⁺.

### Preparation of Intermediate T-5: 5-(trifluoromethyl)pyridin-3-amine

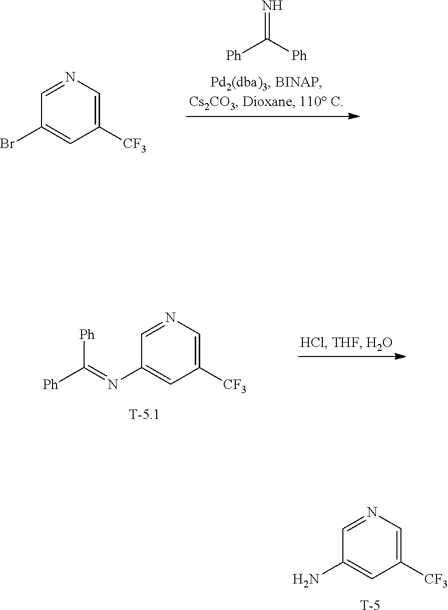

Synthesis of compound T-5.1. To a solution of 3-bromo-5-(trifluoromethyl)pyridine (0.5 g, 2.212 mmol, 1.0 equiv) in 1,4-dioxane (8 mL) was added diphenylmethanimine (0.481 g, 2.65 mmol, 1.2 equiv), cesium carbonate (1.43 g, 4.42 mmol, 2.0 equiv) and (2,2′-bis(diphenylphosphino)-1,1′-binaphthyl) (0.22 g, 0.35 mmol, 0.16 equiv). The reaction mixture was degassed by bubbling through a stream of argon for 10 min. Tris(dibenzylideneacetone) dipalladium(O) (0.202 g, 0.221 mmol, 0.1 equiv) was added, and degassed for 5 min. The reaction mixture was stirred at 110° C. for 6 h. It was cooled to room temperature, filtered through a pad of Celite®. The filtrate was transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford T-5.1 (0.44 g, 26%). MS(ES): m/z 327.32 [M+H]⁺.

Synthesis of compound T-5. To a solution of T-5.1 (0.44 g, 1.35 mmol, 1.0 equiv) in THE (20 mL) was added 4 M hydrochloric acid in 1,4-dioxane (8 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 60% ethyl acetate in hexane) to afford T-5 (0.190 g, 64%). MS(ES): m/z 163.12 [M+H]⁺.

### Preparation of Intermediate T-6: (S)-5-(tert-butyl)-1-(tetrahydrofuran-3-yl)-1H-pyrazol-3-amine

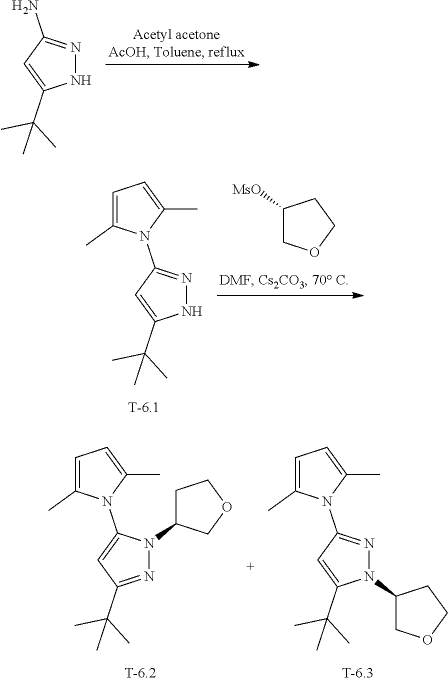

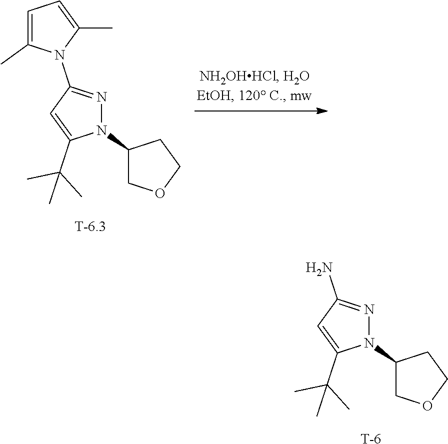

Synthesis of compound T-6.1. To a round-bottomed flask equipped with a Dean-Stark apparatus and a condenser was charged with 5-(tert-butyl)-1H-pyrazol-3-amine (5.0 g, 35.92 mmol, 1.0 equiv), 2, 5-hexanedione (4.09 g, 35.92 mmol, 1.0 equiv), toluene (100 mL) and a few drops of acetic acid (catalytic). The reaction mixture was heated to reflux for 3 hours. It was cooled rt and concentrated under reduced pressure. The residue was purified by flash column chromatography on silica gel (CombiFlash®, 12% ethyl acetate in hexane as eluant) to afford T-6.1. MS (ES): m/z 218.3 [M+H]⁺.

Synthesis of compound T-6.2 and T-6.3. A mixture of T-6.1 (2.5 g, 11.50 mmol, 1.0 equiv), (R)-tetrahydrofuran-3-yl methanesulfonate (1.91 g, 11.50 mmol, 1.0 equiv) and cesium carbonate (7.49 g, 23 mmol, 2.0 equiv) in DMF (15 mL) was stirred at 70° C. for 12 h under nitrogen. It was poured into ice-water, stirred and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2% ethyl acetate in hexane as eluant) to afford T-6.2. MS (ES): m/z 287.4 [M+H]⁺ and T-6.3. MS (ES): m/z 248.3 [M+H]⁺.

Synthesis of compound T-6. To a solution of T-6.3 (0.120 g, 0.417 mmol, 1.0 equiv) in ethanol-water (2:1, 2 mL) was added hydroxylamine hydrochloride (0.287 g, 4.17 mmol, 10 equiv). The reaction mixture was stirred at 120° C. in a microwave reactor for 1 h. It was poured over ice-water, basified by 2 N sodium hydroxide, and extracted with dichloromethane. The combined organic layers were washed with brine, dried over anhydrous sodium sulfate, filtered, and concentrated under reduced pressure to afford T-6. MS (ES): m/z 210.3 [M+H]⁺.

The following intermediates were prepared following the procedure of T-6:

| ID | Structure | Name | MS (ES): m/z |
|---|---|---|---|
| T-7 | 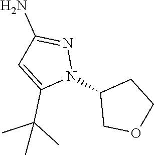 | (R)-5-(tert-butyl)-1-(tetrahydrofuran-3-yl)- 1H-pyrazol-3-amine | 210.3 [M + H]⁺ |
| T-8 | 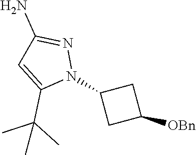 [SMILES: CC(C)(C)c1cc(N)nn1[C@H]1C[C@H](OCc2ccccc2)C1] | trans-1-(3-(benzyloxy)cyclobutyl)-5-(tert- butyl)-1H-pyrazol-3-amine | 300.1 [M + H]⁺ |

### Preparation of Intermediate T-9: 3-amino-5-cyclopropyl-1-(tetrahydro-2H-pyran-3-yl)pyridin-2(1H)-one

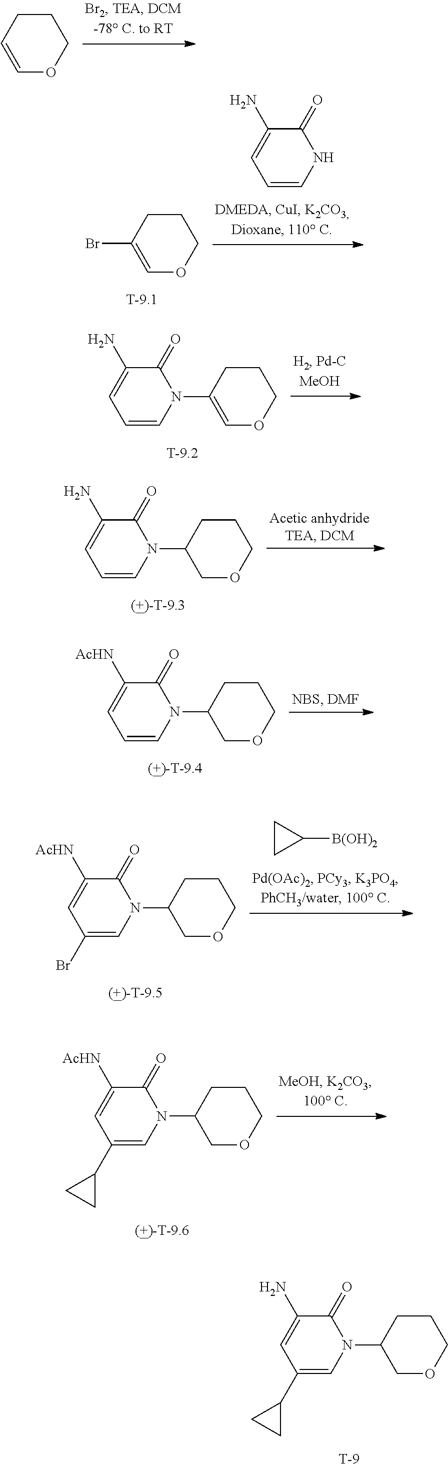

Synthesis of compound T-9.1. To a solution of 3,4-dihydro-2H-pyran (20 g, 238 mmol, 1.0 equiv) in DCM (200 mL) was added bromine (12.2 mL, 238 mmol, 1.0 equiv) in DCM (100 mL) at −78° C. dropwise. The reaction mixture was stirred at −78° C. for 2 h, and it was allowed to warm to room temperature stirring for 16 h. Triethylamine (66 mL, 476 mmol, 2.0 equiv) in DCM (100 mL) was added dropwise at room temperature and then stirred for 5 h. The reaction mixture was concentrated under reduced pressure to afford residue and diethyl ether was added, solid was removed by filtration. The filtrate was concentrated under reduced pressure to afford residue which was purified by vacuum distillation (80° C., 0.02 mmHg), to afford T-9.1. ¹H NMR (400 M Hz, CDCl₃): 6.68 (s, 1H), 4.02-4.00 (t, J=4 Hz, 2H), 2.45-2.42 (m, 2H), 2.06-2.00 (m, 2H).

Synthesis of compound T-9.2. To a mixture of T-9.1 (8 g, 49.08 mmol, 1.0 equiv), 3-aminopyridin-2(1H)-one (6.48 g, 58.89 mmol, 1.2 equiv) and potassium carbonate (13.6 g, 98.16 mmol, 2.0 equiv) in 1,4-dioxane (100 mL) was degassed by bubbling through a stream of argon for 15 min. Copper iodide (1.4 g, 7.40 mmol, 0.15 equiv) and 1,2-dimethylethylenediamine (1.60 mL, 14.72 mmol, 0.30 equiv) were added. The reaction mixture was degassed for 5 min and stirred at 110° C. for 12 h. It was cooled to room temperature, transferred into water and extracted with ethyl acetate. The combined organic layers were washed with brine, dried over anhydrous sodium sulfate, filtered and concentrated under reduced pressure. The residue was purified by flash column chromatography on silica gel (CombiFlash®, 1.2% methanol in DCM) to afford T-9.2. MS(ES): m/z 193.09 [M+H]⁺.

Synthesis of compound (±)-T-9.3. A mixture of T-9.2 (8 g, 41.62 mmol, 1.0 equiv) and palladium on charcoal (4 g) in methanol (100 mL) was stirred under hydrogen (1 atm) for 16 h at room temperature. It was filtered through a pad of Celite® and rinsed with methanol. The filtrate was concentrated under reduced pressure. The residue was purified by trituration with n-pentane to afford (±)-T-9.3. MS (ES): m/z 195.11 [M+H]⁺.

Synthesis of compound (±)-T-9.4. To a solution of (±)-T-9.3 (2 g, 10.30 mmol, 1.0 equiv) in DCM (20 mL) at 0° C. was added triethylamine (4.33 mL, 30.9 mmol, 3.0 equiv) and acetic anhydride (1.55 mL, 16.49 mmol, 1.6 equiv). The reaction mixture was stirred at room temperature for 2 h. It was transferred into water and extracted with DCM. The combined organic layers were dried over anhydrous sodium sulfate, filtered and concentrated under reduced pressure. The residue was purified by flash column chromatography on silica gel (CombiFlash®, 0-5% methanol in DCM) to afford (±)-T-9.4. MS (ES): m/z 237.12 [M+H]⁺.

Synthesis of compound (±)-T-9.5. To a solution of (±)-T-9.4 (0.9 g, 3.81 mmol, 1.0 equiv) in DMF (10 mL) was added NBS (1.017 g, 5.71 mmol, 1.5 equiv) and stirred at room temperature for 1 h. It was poured into ice-water and extracted with ethyl acetate. The combined organic layers were washed with brine, dried over anhydrous sodium sulfate, filtered and concentrated under reduced pressure. The residue was purified by flash column chromatography on silica gel (CombiFlash®, 60% ethyl acetate in hexane) to afford (±)-T-9.5. MS(ES): m/z 316.2 [M+H]⁺.

Synthesis of compound (±)-T-9.6. A mixture of (±)-T-9.5 (0.345 g, 1.09 mmol, 1.0 equiv), cyclopropylboronic acid (0.282 g, 3.28 mmol, 3.0 equiv), potassium carbonate (0.300 g, 2.18 mmol, 2.0 equiv) and tricyclohexylphosphine (0.061 g, 0.218 mmol, 0.2 equiv) in toluene (10 mL) and water (2 mL) was degassed by bubbling through a stream of argon for 10 min. Palladium(II) acetate (0.019 g, 0.087 mmol, 0.08 equiv) was added and degassed for 5 min. The reaction mixture was stirred at 110° C. for 2 h. It was cooled to room temperature, filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 100% ethyl acetate) to afford (±)-T-9.6 (0.182 g, 60%). MS(ES): m/z 277.3 [M+H]⁺.

Synthesis of compound T-9. To a solution of compound (±)-T-9.6 (0.182 g, 0.658 mmol, 1.0 equiv) in methanol (10 mL) was added potassium carbonate (1.816 g, 13.16 mmol, 20.0 equiv) and stirred at 100° C. for 16 h. It was transferred into water, extracted with ethyl acetate. The combined organic layers were washed with brine, dried over sodium sulfate, filtered and concentrated under reduced pressure. The residue was purified by flash column chromatography on silica gel (CombiFlash®, 2.0% methanol in dichloromethane) to afford T-9 (0.114 g, 74%). MS(ES): m/z 235.3 [M+H]⁺.

### Preparation of Intermediate T-10: (R)-1-(tetrahydrofuran-3-yl)-5-(trifluoromethyl)-1H-pyrazol-3-amine

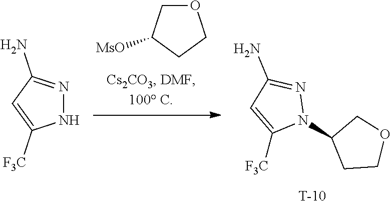

Synthesis of compound T-10. To a mixture of 5-(trifluoromethyl)-1H-pyrazol-3-amine (1.0 g, 6.62 mmol, 1.0 equiv), (S)-tetrahydrofuran-3-yl methanesulfonate (2.20 g, 13.24 mmol, 2.0 equiv) and cesium carbonate (6.45 g, 19.86 mmol, 3.0 equiv) in DMF (10 mL) was stirred at 100° C. for 12 h. It was transferred into ice-water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford T-10. MS (ES): m/z 222.1 [M+H]⁺.

### Preparation of Intermediate T-11: (S)-1-(tetrahydrofuran-3-yl)-5-(trifluoromethyl)-1H-pyrazol-3-amine

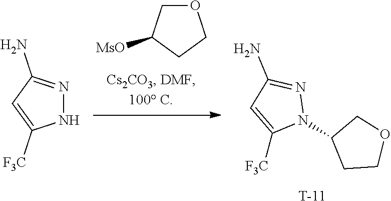

Synthesis of compound T-11. Compound T-11 was prepared following the procedure described in the synthesis of T-10. The product was purified by flash column chromatography on silica gel (CombiFlash®, 20% ethyl acetate in hexane). MS (ES): m/z 222.1 [M+H]⁺.

### Preparation of Intermediate T-12: 4-((4-ethylpiperazin-1-yl)methyl)-3-(trifluoromethyl)aniline

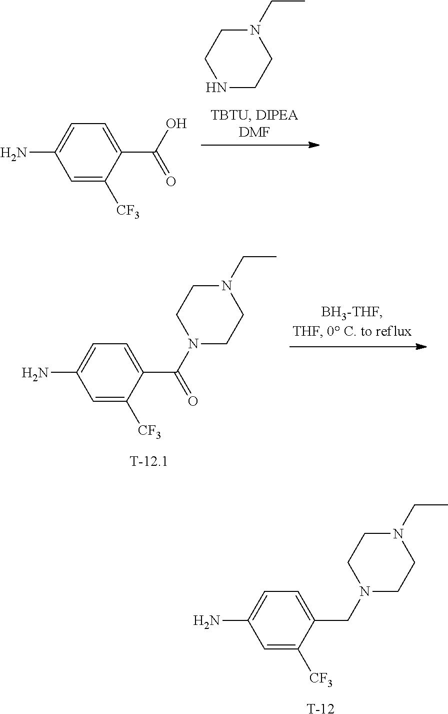

Synthesis of compound T-12.1. To a solution of 4-amino-2-(trifluoromethyl)benzoic acid (10 g, 48.75 mmol, 1.0 equiv) in DMF (30 mL) was added O-(benzotriazol-1-yl)-N,N,N′,N′-tetramethyluronium tetrafluoroborate (18.4 g, 58.5 mmol, 1.2 equiv) at 0° C. and the reaction mixture was stirred for 30 min. To the mixture was added 1-ethylpiperazine (6.67 g, 58.5 mmol, 1.2 equiv), followed by N, N-diisopropylethylamine (24 mL, 146.25 mmol, 3.0 equiv) and stirred at room temperature for 3 h. It was transferred into ice cold water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.5% methanol in DCM) to afford T-12.1 (9 g, 61%). MS (ES): m/z 302.3 [M+H]⁺.

Synthesis of compound T-12. To a solution of T-12.1 (2 g, 6.64 mmol, 1.0 equiv) in THF (20 mL) was added lithium aluminium hydride solution (1 M in THF, 19.9 mL, 19.92 mmol, 3.0 equiv) at 0° C. The reaction mixture was stirred at 80° C. for 3 h. It was cooled to room temperature, transferred into ice-water, and extracted with ethyl acetate. The combined organic layers were washed with brine, dried over anhydrous sodium sulfate, filtered, and concentrated under reduced pressure to afford T-12 (1.3 g, 68%). MS(ES): m/z 288.3 [M+H]⁺.

### Preparation of Provided Compounds

### Example 1: N-(3-methyl-5-(trifluoromethyl)phenyl)-2-(1-(pyrazin-2-yl)piperidin-4-yl)acetamide

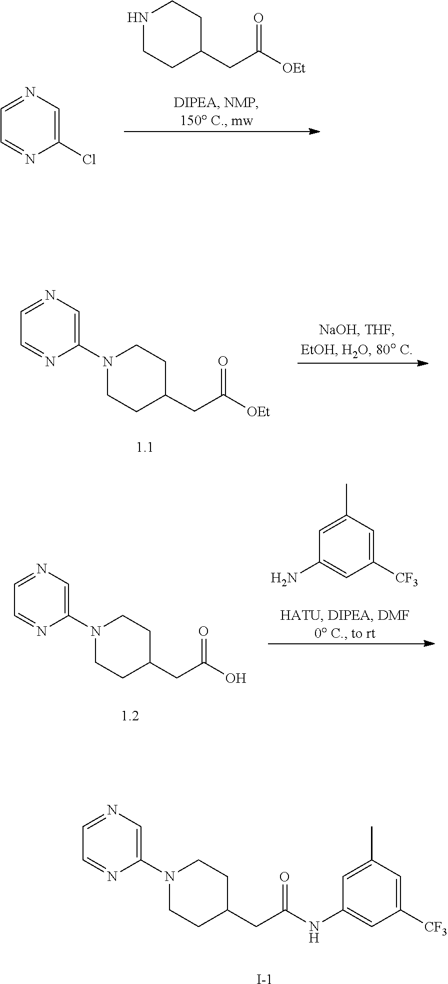

Synthesis of compound 1.1. A solution of 2-chloropyrazine (0.5 g, 4.37 mmol, 1.0 equiv), ethyl 2-(piperidin-4-yl)acetate (1.12 g, 6.55 mmol, 1.5 equiv) and N,N-diisopropylethylamine (1.13 g 8.77 mmol, 2.0 equiv) in N-methyl-2-pyrrolidone (10 mL) was stirred at 150° C. in a microwave reactor for 30 min. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford 1.1 (0.350 g, 32%). MS(ES): m/z 250.6 and 251.23 [M+H]⁺.

Synthesis of compound 1.2. To a solution of 1.1 (0.350 g, 1.4 mmol, 1.0 equiv) in a mixture of THE:ethanol:water (8 mL:4 mL:4 mL) was added sodium hydroxide (0.294 g, 7 mmol, 5.0 equiv). The reaction mixture was stirred at 80° C. for 1 h. The reaction mixture was concentrated under reduced pressure. The residue was dissolved in water and acidified with dilute hydrochloric acid at 0° C. The solids were collected by filtration and dried under reduced pressure to afford 1.2 (0.210 g, 68%). MS(ES): m/z 221.8 and 223.2 [M+H]⁺.

Synthesis of I-1. A solution of 1.2 (0.210 g, 0.949 mmol, 1.0 equiv) and HATU (0.539 g, 1.418 mmol, 1.5 equiv) in DMF (5 mL) was stirred at 0° C. for 15 min. To the solution was added 3-methyl-5-(trifluoromethyl)aniline (0.249 g, 1.42 mmol, 1.5 equiv) and N,N-diisopropylethylamine (0.366 g, 2.837 mmol, 3.0 equiv). The reaction mixture was stirred at room temperature for 16 h, transferred into ice-water and extracted with ethyl acetate. The combined organic layers were washed with water and brine, dried over anhydrous sodium sulfate, filtered, and concentrated under reduced pressure. The residue was purified by flash column chromatography on silica gel (CombiFlash®, 2% methanol in DCM) to afford I-1 (0.060 g, 17%). MS(ES): m/z: 379.26 [M+H]⁺; ¹H NMR (DMSO-d₆, 400 MHz): δ 10.17 (s, 1H), 8.31 (s, 1H), 8.05 (s, 1H), 7.89 (s, 1H), 7.78 (d, J=2.4 Hz, 1H), 7.61 (s, 1H), 7.22 (s, 1H), 4.32 (m, 2H), 2.88 (m, 2H), 2.36 (s, 3H), 2.28 (m, 2H), 2.07 (m, 1H), 1.76 (m, 2H), 1.27-1.17 (m, 2H).

### Example 2: 2-(1-(5-acetamidopyrazin-2-yl)piperidin-4-yl)-N-(3-methyl-5-(trifluoromethyl)phenyl)acetamide

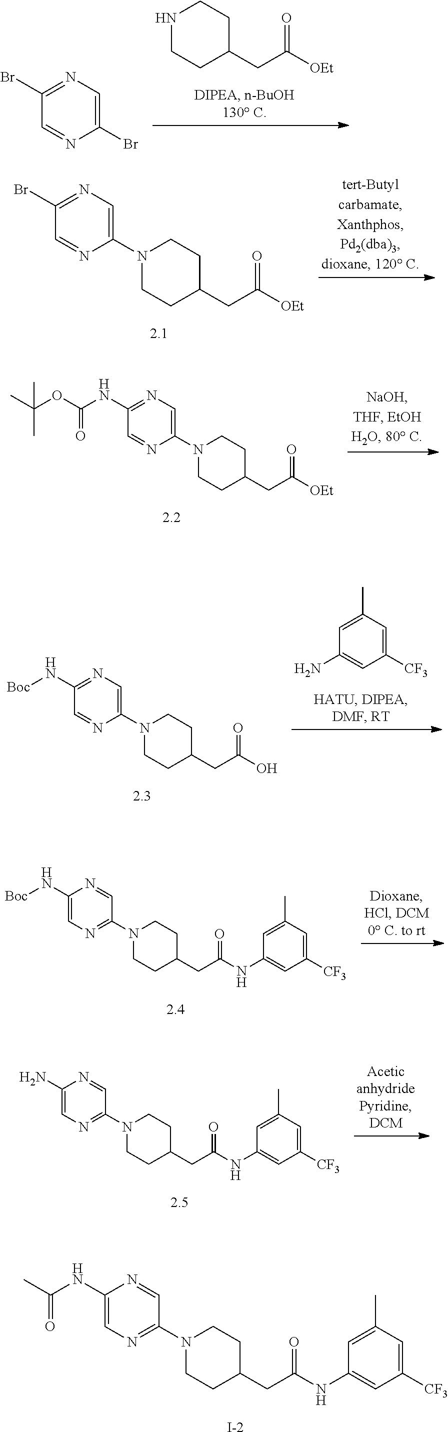

Synthesis of compound 2.1. A solution of 2,5-dibromopyrazine (2.0 g, 8.438 mmol, 1.0 equiv), ethyl 2-(piperidin-4-yl)acetate (2.28 g, 16.877 mmol, 2.0 equiv) and N,N-diisopropylethylamine (3.26 g, 25.316 mmol, 3.0 equiv) in n-butanol (15 mL) was stirred at 130° C. for 30 min. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% ethyl acetate in hexane) to afford 2.1 (1.90 g, 69%). MS(ES): m/z 328.12 and 330.12 [M+1]⁺.

Synthesis of compound 2.2. A mixture of 2.1 (1.9 g, 5.792 mmol, 1.0 equiv), tert-butyl carbamate (1.35 g, 11.585 mmol, 2.0 equiv) and cesium carbonate (5.647 g, 17.378 mmol, 3.0 equiv)) in dioxane (20 mL) was degassed by bubbling through a stream of argon for 10 min. 4,5-Bis(diphenylphosphino)-9,9-dimethylxanthene (0.669 g 1.158 mmol, 0.2 equiv) and tris(dibenzylideneacetone)dipalladium(O) (0.530 g 0.579 mmol, 0.1 equiv) were added and degassed for another 5 min. The reaction mixture was stirred at 130° C. for 3 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford 2.2 (0.800 g, 38%). MS(ES): m/z 365.1 [M+H]⁺.

Synthesis of compound 2.3. To a solution of 2.2 (0.800 g, 1.4 mmol, 1.0 equiv) in THE:ethanol:water (10 mL:5 mL:5 mL) was added sodium hydroxide (0.461 g, 10.989 mmol, 5.0 equiv). The reaction mixture was stirred at 80° C. for 1 h. The reaction mixture was concentrated under reduced pressure. The residue was dissolved in water and acidified with dilute hydrochloric acid at 0° C. The solids were collected by filtration and dried under reduced pressure to afford 2.3 (0.600 g, 81%). MS(ES): m/z 336.39 [M+H]⁺.

Synthesis of compound 2.4. To a solution of 2.3 (0.400 g, 1.190 mmol, 1.0 equiv) in DMF (6 mL) at 0° C. and added HATU (0.678 g, 1.785 mmol, 1.5 equiv). The reaction mixture was stirred for 15 min, and 3-methyl-5-(trifluoromethyl)aniline (0.321 g, 1.785 mmol, 1.5 equiv) and N,N-diisopropylethylamine (0.460 g, 3.571 mmol, 3.0 equiv) were added. The reaction mixture was stirred at room temperature for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford 2.4 (0.300 g, 51%). MS(ES): m/z: 494.33 [M+H]⁺.

Synthesis of compound 2.5. To a solution of 2.4 (0.300 g, 0.608 mmol, 1.0 equiv) in DCM (10 mL) at 0° C. and added a solution of 4 M hydrogen chloride in dioxane (5 mL). The reaction mixture was stirred at room temperature for 1 h. It was transferred into ice-water, neutralized with sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford 2.5 (0.140 g, 59%). MS(ES): m/z: 394.12 [M+H]⁺.

Synthesis of I-2. To a solution of 2.5 (0.075 g, 0.190 mmol, 1.0 equiv) in DCM (3 mL) at 0° C. was added pyridine (0.150 g, 1.908 mmol, 10 equiv) and acetic anhydride (0.192 g, 1.908 mmol, 10 equiv). The reaction mixture was stirred at room temperature for 1 h. It was transferred into ice-water and extracted with DCM. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford I-2 (0.022 g, 26.5%). MS(ES): m/z: 436.39 [M+H]⁺, ¹H NMR (DMSO-d₆, 400 MHz): δ 10.29 (s, 1H), 10.19 (s, 1H), 8.76 (s, 1H), 8.08 (s, 1H), 7.91 (s, 1H), 7.63 (s, 1H), 7.23 (s, 1H), 4.23 (m, 2H), 2.85 (m, 2H), 2.38 (s, 3H), 2.35 (m, 2H), 2.06 (s, 3H), 1.76 (m, 2H), 1.25 (m, 3H).

### Example 3: 2-(1-(5-acetamidopyrazin-2-yl)piperidin-4-yl)-N-(1-methyl-2-oxo-5-(trifluoromethyl)-1,2-dihydropyridin-3-yl)acetamide

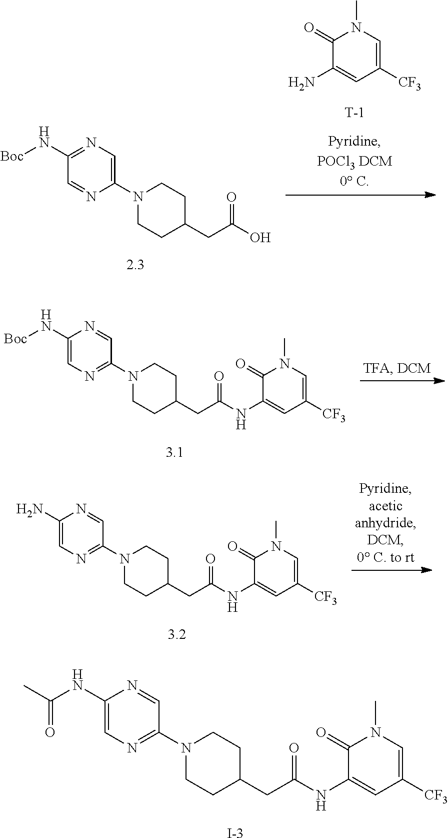

Synthesis of compound 3.1. To a solution of 2.3 (0.325 g, 0.964 mmol, 1.0 equiv), T-1 (0.185 g, 0.964 mmol, 1.0 equiv), and pyridine (0.611 g, 7.738 mmol, 8.0 equiv) in DCM (15 mL) at 0° C. was added phosphorus oxychloride (0.739 g, 4.836 mmol, 5.0 equiv). The reaction mixture was stirred at 0° C. for 15 min. It was transferred into ice-water, neutralized with sodium bicarbonate, and extracted with DCM. The combined organic layers were washed with brine, dried over anhydrous sodium sulfate, filtered, and concentrated under reduced pressure. The residue was purified by flash column chromatography on silica gel (CombiFlash®, 45% ethyl acetate in hexane) to afford 3.1 (0.170 g, 34%). MS(ES): m/z: 511.34 [M+H]⁺.

Synthesis of compound 3.2. To a solution of 3.1 (0.170 g, 0.332 mmol, 1.0 equiv) in DCM (3 mL) at 0° C. was added trifluoroacetic acid (0.6 mL). The reaction mixture was stirred at room temperature for 30 min. It was transferred into ice-water, neutralized with sodium bicarbonate, and extracted with DCM. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford 3.2 (0.080 g, 59%). MS(ES): m/z: 411.32 [M+H]⁺.

Synthesis of I-3. Compound I-3 was prepared from compound 3.2 following the procedure described in the synthesis of I-2. The product was purified by flash column chromatography on silica gel (CombiFlash®, 2.5% methanol in DCM). MS(ES): m/z: 453.33 [M+H]⁺, ¹H NMR (DMSO-d₆, 400 MHz): δ 10.28 (s, 1H), 9.57 (s, 1H), 8.74 (s, 1H), 8.46 (bs, 1H), 8.13 (s, 1H), 8.06 (s, 1H), 4.20 (m, 2H), 3.57 (s, 3H), 2.82 (m, 2H), 2.50-2.46 (m, 2H), 2.04-1.91 (m, 4H), 1.70 (m, 2H), 1.26-1.18 (m, 2H).

### Example 4: N-(3-methyl-5-(trifluoromethyl)phenyl)-1-(1-(pyrazin-2-yl)piperidin-4-yl)cyclopropane-1-carboxamide

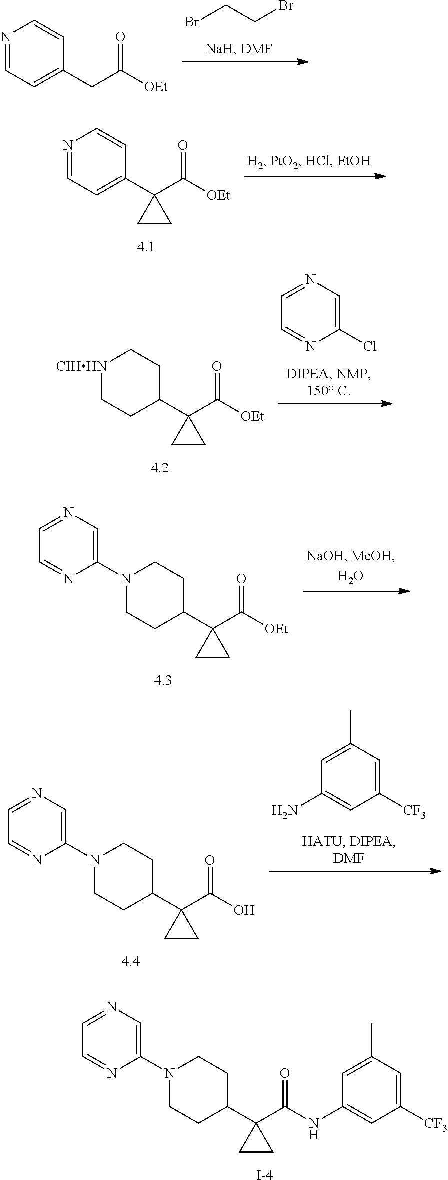

Synthesis of compound 4.1. To a solution of ethyl 2-(pyridin-4-yl)acetate (1.0 g, 6.05 mmol, 1.0 equiv) in DMF (50 mL) was added sodium hydride (0.174 g, 7.27 mmol, 1.2 equiv) at 0° C. and stirred for 15 min. To the mixture was added 1,2-dibromoethane (1.36 g, 7.27 mmol, 1.2 equiv) and stirred at room temperature for 2 h. It was transferred into ice-water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford 4.1 (900 mg, 78%). MS (ES): m/z 192.11 [M+H]⁺.

Synthesis of compound 4.2. A mixture of 4.1 (900 mg, 4.71 mmol, 1.0 equiv), concentrated hydrogen chloride (36.5 wt %, 0.5 mL) and platinum oxide (0.375 g) in ethanol (20 mL) was stirred under hydrogen (1 atm) for 14 h. The reaction mixture was filtered through a pad of Celite® and rinsed with methanol. The filtrate was concentrated under reduced pressure to afford 4.2 (0.7 g, 64%). MS (ES): m/z 198.12 [M+H]⁺.

Synthesis of compound 4.3. A solution of 4.2 (0.600 g, 2.57 mmol, 1.0 equiv), 2-chloropyrazine (0.322 g, 2.83 mmol, 1.1 equiv) and N,N-diisopropylethylamine (1.34 mL, 7.72 mmol, 3.0 equiv) in N-methyl-2-pyrrolidinone (10 mL) was stirred at 150° C. in a microwave reactor for 1 h. It was cooled to room temperature, transferred into ice-water, and extracted with ethyl acetate. The combined organic layers were washed with brine, dried over anhydrous sodium sulfate, filtered, and concentrated under reduced pressure to afford crude material this was purified by flash column chromatography on silica gel (CombiFlash®, 3% methanol in DCM) to afford 4.3 (0.600 g, 57%). MS (ES): m/z 276.1 [M+H]⁺.

Synthesis of compound 4.4. To a solution of 4.3 (0.600 g, 2.18 mmol, 1.0 equiv) in methanol (5 mL) was added a solution of sodium hydroxide (0.872 g, 21.80 mmol, 10 equiv) in water (2 mL), and the reaction mixture was heated at 60° C. for 3 h. It was concentrated under reduced pressure. To the residue was added water and acidified with 1 N hydrochloric acid to adjust to pH 6-6.5. The mixture was extracted with 10% methanol in DCM. The combined organic layers were washed with brine, dried over anhydrous sodium sulfate, filtered, and concentrated under reduced pressure to afford 4.4 (0.300 g, 56%). MS (ES): m/z 248.1 [M+H]⁺.

Synthesis of compound I-4. To a solution of 4.4 (0.150 g, 0.607 mmol, 1.0 equiv) in DMF (5 mL) was added HATU (0.276 g, 0.728 mmol, 1.5 equiv) and stirred at room temperature for 30 min. To the mixture was added N, N-diisopropylethylamine (0.234 g, 1.82 mmol, 3.0 equiv), followed by 3-methyl-5-(trifluoromethyl)aniline (0.077 mg 0.728 mmol 1.2 equiv). The reaction mixture was stirred at room temperature for 16 h. It was transferred into ice-water and extracted with ethyl acetate. The combined organic layers were washed with brine, dried over anhydrous sodium sulfate, filtered, and concentrated under reduced pressure. The residue was purified flash column chromatography on silica gel (CombiFlash®, 3% methanol in DCM) to afford I-4. MS(ES): m/z 405.43 [M+H]⁺; ¹H NMR (DMSO-d₆, 400 MHz): 9.64 (s, 1H), 8.30 (s, 1H), 8.03 (s, 1H), 7.86 (s, 1H), 7.76 (d, J=2.4 Hz, 1H), 7.73 (s, 1H), 7.21 (s, 1H), 4.43 (m, 2H), 2.81-2.73 (m, 2H), 2.35 (s, 3H), 1.94 (m, 1H), 1.72 (m, 2H), 1.32-1.23 (m, 2H), 0.95 (s, 2H), 0.75 (s, 2H).

### Example 5: N-(1-methyl-2-oxo-5-(trifluoromethyl)-1,2-dihydropyridin-3-yl)-1-(1-(pyrazin-2-yl)piperidin-4-yl)cyclopropane-1-carboxamide

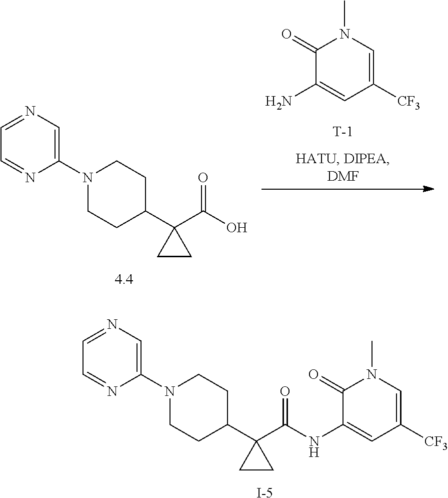

Synthesis of compound I-5. Compound I-5 was prepared from compound 4.4 and T-1 following the procedure described in the synthesis of 1-4. The product was purified flash column chromatography on silica gel (CombiFlash®, 3.9% methanol in DCM). MS(ES): m/z 422 [M+H]⁺; ¹H NMR (DMSO-d₆, 400 MHz): 8.75 (s, 1H), 8.32 (s, 1H), 8.28 (s, 1H), 8.16 (s, 1H), 8.05 (s, 1H), 7.79 (s, 1H), 7.21 (s, 1H), 4.43 (m, 2H), 3.59 (s, 3H), 2.81 (m, 2H), 1.92 (m, 1H), 1.74 (m, 2H), 1.45-1.15 (m, 2H), 0.98 (s, 2H), 0.87 (s, 2H).

### Example 6: 2-(1-(5-acetamidopyrazin-2-yl)piperidin-4-yl)-N-(3-(trifluoromethyl)phenyl)acetamide

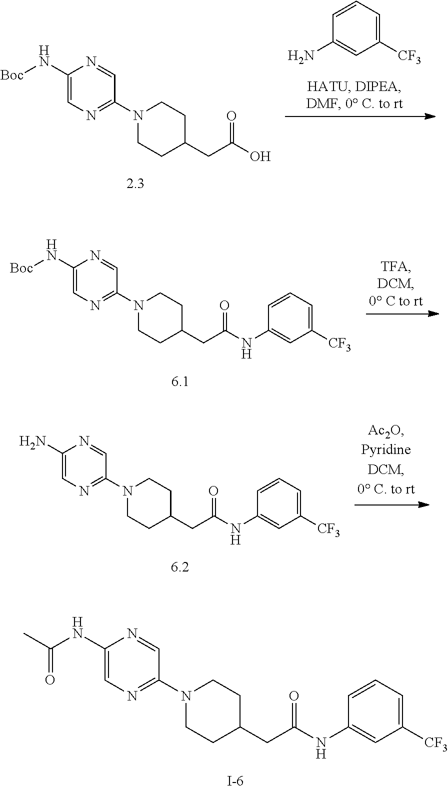

Synthesis of compound 6.1. Compound 6.1 was prepared from compound 2.3 and 3-(trifluoromethyl)aniline following the procedure described in the synthesis of 1-4. The product was purified by flash column chromatography on silica gel (CombiFlash®, 30% ethyl acetate in hexane). MS(ES): m/z: 480.22 [M+H]⁺.

Synthesis of compound 6.2. To a solution of 6.1 (0.170 g, 0.354 mmol, 1.0 equiv) in DCM (5 mL) at 0° C. was added trifluoroacetic acid (1.0 mL). The reaction mixture was stirred at room temperature for 30 min. It was transferred slowly into a mixture of ice and sodium bicarbonate solution and extracted with DCM. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford 6.2 (0.110 g, 82%). MS(ES): m/z: 380.18 [M+H]⁺.

Synthesis of I-6. Compound I-6 was prepared from compound 6.2 following the procedure described in the synthesis of I-2. The product was purified by flash column chromatography on silica gel (CombiFlash®, 2.5% methanol in DCM). MS(ES): m/z: 422.1 [M+H]⁺; ¹H NMR (DMSO-d₆, 400 MHz): δ 10.30 (s, 1H), 10.28 (s, 1H), 8.76 (s, 1H), 8.14 (s, 1H), 8.08 (s, 1H), 7.91 (d, J 8 Hz, 1H), 7.55 (t, J=8 Hz, 1H), 7.40 (d, J=8 Hz, 1H), 7.23 (s, 1H), 4.25 (m, 2H), 2.85 (m, 2H), 2.31 (m, 2H), 2.06 (s, 3H), 1.76 (m, 2H), 1.29-1.23 (m, 3H).

### Example 7: 2-(1-(5-acetamidopyrazin-2-yl)piperidin-4-yl)-N-(3-((4-methylpiperazin-1-yl)methyl)-5-(trifluoromethyl)phenyl)acetamide

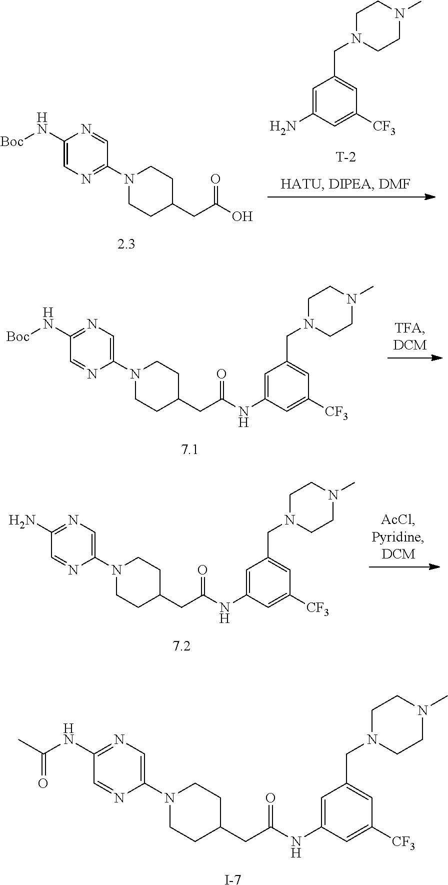

Synthesis of compound 7.1. Compound 7.1 was prepared from compound 2.3 and T-2, following the procedure described in the synthesis of I-4. The product was purified by flash column chromatography on silica gel (CombiFlash®, 3.8% methanol in DCM). MS(ES): m/z: 592.3 [M+H]⁺.

Synthesis of compound 7.2. To a solution of 7.1 (0.220 g, 0.332 mmol, 1.0 equiv) in DCM (3 mL) at 0° C. was added trifluoroacetic acid (0.6 mL). The reaction mixture was stirred at room temperature for 30 min. After completion of reaction, the reaction mixture was extracted with DCM. The combined organic layers were washed with brine, dried over anhydrous sodium sulfate, filtered, and concentrated under reduced pressure. The residue was purified by flash column chromatography on silica gel (CombiFlash®, 6.9% methanol in DCM) to afford 7.2 (0.110 g, 60.18%). MS(ES): m/z: 491.5 [M+H]⁺.

Synthesis of 1-7: Compound 1-7 was prepared from compound 7.2 following the procedure described in the synthesis of 1-2. The product was purified by flash column chromatography on silica gel (CombiFlash®, 3.7% methanol in DCM). MS(ES): m/z: 534.0 and 535.6 [M+H]⁺, ¹H NMR (DMSO-d₆, 400 MHz): δ 10.30 (s, 1H), 10.29 (s, 1H), 8.76 (s, 1H), 8.08 (s, 2H), 7.73 (s, 1H), 7.30 (s, 1H), 4.23 (m, 2H), 3.52 (s, 2H), 2.85 (m, 2H), 2.45-2.25 (m, 8H), 2.21 (s, 3H), 2.06 (s, 3H), 1.76 (m, 2H), 1.24 (m, 4H).

### Example 8: 1-methyl-3-(3-methyl-5-(trifluoromethyl)phenyl)-1-(1-(pyrazin-2-yl)piperidin-4-yl)urea

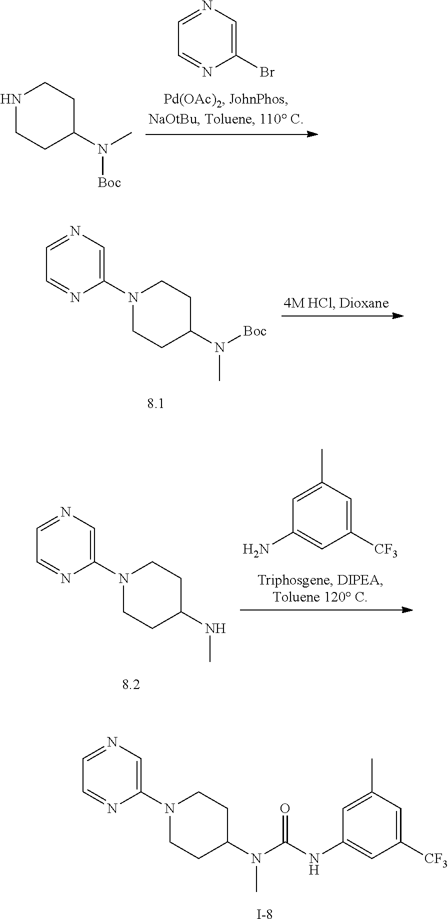

Synthesis of compound 8.1. A mixture of tert-butyl methyl(piperidin-4-yl)carbamate (0.700 g, 3.27 mmol, 1.0 equiv) and 2-bromopyrazine (1.0 g, 6.54 mmol, 2.0 equiv) in toluene (10 mL) was degassed by bubbling through a stream of argon for 15 min. Under argon atmosphere, 2-(di-tert-butylphosphino)biphenyl (0.05 g, 0.165 mmol, 0.05 equiv), sodium tert-butoxide (0.63 g, 6.54 mmol, 2.0 equiv), and palladium(II) acetate (0.021 g, 0.165 mmol, 0.05 equiv) were added to the reaction mixture and degassed for another 5 min. The reaction mixture was stirred at 100° C. for 24 h. It was cooled to room temperature, transferred into brine, and extracted with DCM. The combined organic layers were dried over anhydrous sodium sulfate, filtered, and concentrated under reduced pressure. The residue was purified by flash column chromatography on silica gel (CombiFlash®, 1% methanol in DCM) to afford 8.1 (0.451 g, 47%). MS (ES): m/z: 293.16 [M+H]⁺.

Synthesis of compound 8.2. To a solution of 8.1 (0.31 g, 1.06 mmol, 1.0 equiv) in DCM (5 mL) was added hydrochloric acid (4.0 M in dioxane, 3.0 mL) at 0° C. The reaction mixture was stirred at room temperature for 2 h. It was concentrated under reduced pressure to afford 8.2 (0.28 g). It was used in the next step without purification. MS(ES): m/z 193.19 [M+H]⁺.

Synthesis of I-8. To a solution of 8.2 (0.212 g, 1.21 mmol, 1.0 equiv) in toluene (5 mL) was added N,N-diisopropylethylamine (1.0 mL, 6.0 mmol, 5.0 equiv), followed by triphosgene (0.126 g, 0.35 mmol, 0.35 equiv) at 0° C. The reaction mixture was stirred at 120° C. for 2 h. It was cooled room temperature and 3-methyl-5-(trifluoromethyl)aniline (0.28 g, 1.45 mmol, 1.2 equiv) was added. The mixture was stirred at room temperature for 16 h. It was poured into ice-water and extracted with DCM. The combined organic layers were washed with brine, dried over anhydrous sodium sulfate, filtered, and concentrated under reduced pressure. The residue was purified by flash column chromatography on silica gel (CombiFlash®, 2-3% methanol in DCM) to afford I-8 (0.175 g, 22%). MS (ES): m/z 394.25 [M+H]⁺, ¹H NMR (DMSO-d₆, 400 MHz): δ 8.51 (s, 1H), 8.35 (s, 1H), 8.06 (s, 1H), 7.76 (d, 2H), 7.58 (s, 1H), 7.08 (s, 1H), 4.47 (m, 2H), 4.31 (m, 1H), 2.89 (m, 2H), 2.78 (s, 3H), 2.37 (s, 3H), 1.65 (m, 4H).

### Example 9: 3-(3-(4-hydroxypiperidin-1-yl)-5-(trifluoromethyl)phenyl)-1-methyl-1-(1-(pyrazin-2-yl)piperidin-4-yl)urea

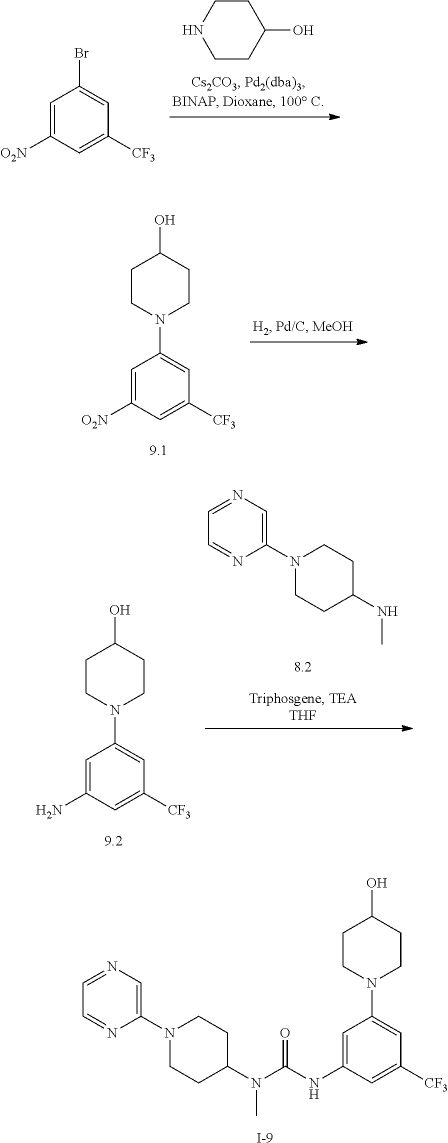

Synthesis of compound 9.1. A mixture of 1-bromo-3-nitro-5-(trifluoromethyl)benzene (1.0 g, 3.70 mmol, 1.0 equiv), piperidin-4-ol (0.748 g, 7.40 mmol, 2.0 equiv) and cesium carbonate (3.01 g, 9.25 mmol, 2.5 equiv) in 1,4-dioxane (20 mL) was degassed by bubbling through a stream of argon under argon atmosphere for 10 min. 2,2′-Bis(diphenylphosphino)1,1′binaphthyl (0.230 g, 0.740 mmol, 0.2 equiv) and tris (dibenzylideneacetone)dipalladium (O) (0.338 g, 0.370 mmol, 0.1 equiv) was added and degassed for another 5 min. The reaction mixture was stirred at 110° C. for 5 h. It was cooled to room temperature, filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10-15% ethyl acetate in hexane) to afford 9.1 (0.500, 46.5%). MS(ES): m/z 291 [M+H]⁺.

Synthesis of compound 9.2. A mixture of compound 9.1 (0.500 g, 1.72 mmol, 1.0 equiv) and 10% palladium on carbon (0.2 g) in methanol (5 mL) was stirred under hydrogen (1 atm) for 2 h. It was filtered through a pad of Celite® and rinsed with methanol. The filtrate was concentrated under reduced pressure. The residue was purified by flash column chromatography on silica gel (CombiFlash®, 5% methanol in DCM as eluent) to afford 9.2 (0.350 g, 78%). MS(ES): m/z 261.26 [M+H]⁺.

Synthesis of compound I-9. To a solution of compound 9.2 (0.150 g, 0.780 mmol, 1.0 equiv) and triethylamine (0.315 g, 3.12 mmol, 4 equiv) in THE (5 mL) at 0° C. was added a solution of triphosgene (0.092 g, 0.312 mmol, 0.4 equiv) in THE (0.5 mL) dropwise and stirred for 15 min. To the mixture was added triethylamine (0.630 g, 6.24 mmol, 8 equiv), followed by 8.2 (0.203 g, 0.40 mmol, 1.0 equiv). The reaction mixture was stirred at room temperature for 30 min. It was transferred into water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.6% methanol in DCM) to afford I-9. MS(ES): m/z 479.39 [M+H]⁺; ¹H NMR (DMSO-d₆, 400 MHz): 8.37 (s, 2H), 8.07 (s, 1H), 7.81 (s, 1H), 7.39 (s, 1H), 7.35 (s, 1H), 6.75 (s, 1H), 4.72 (m, 1H), 4.49 (m, 2H), 4.34 (m, 1H), 3.64 (m, 1H), 3.54 (m, 2H), 2.92 (m, 4H), 1.81 (m, 2H), 1.66 (m, 4H), 1.46 (m, 2H), 1.29-1.18 (m, 2H).

### Example 10: 1-methyl-3-(1-methyl-2-oxo-5-(trifluoromethyl)-1,2-dihydropyridin-3-yl)-1-(1-(pyrazin-2-yl)piperidin-4-yl)urea

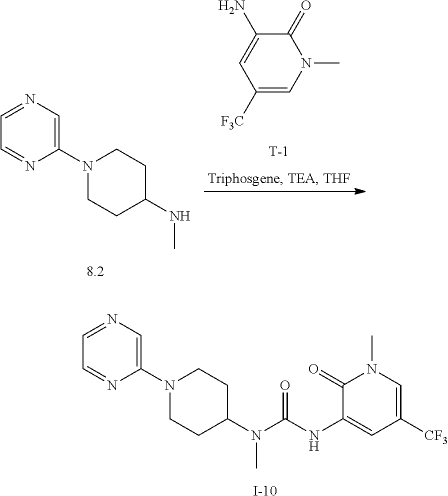

Synthesis of compound 1-10. Compound 1-10 was prepared from compound 8.2 and T-1 following the procedure described in the synthesis of I-9. The product was purified by flash column chromatography on silica gel (CombiFlash®, 2% methanol in DCM). MS(ES): m/z 411.30 [M+H]⁺; ¹H NMR (DMSO-d₆, 400 MHz): 8.370 (s, 1H), 8.17 (d, J=2 Hz, 1H), 8.08 (s, 1H), 8.04 (s, 1H), 7.90 (s, 1H), 7.81 (d, J=2.4 Hz, 1H), 4.49 (m, 2H), 4.33 (m, 1H), 3.59 (s, 3H), 2.93 (m, 2H), 2.83 (s, 3H), 1.68 (m, 4H).

### Example 11: 1-(1-(5H-pyrrolo[2,3-b]pyrazin-2-yl)piperidin-4-yl)-1-methyl-3-(3-methyl-5-(trifluoromethyl)phenyl)urea

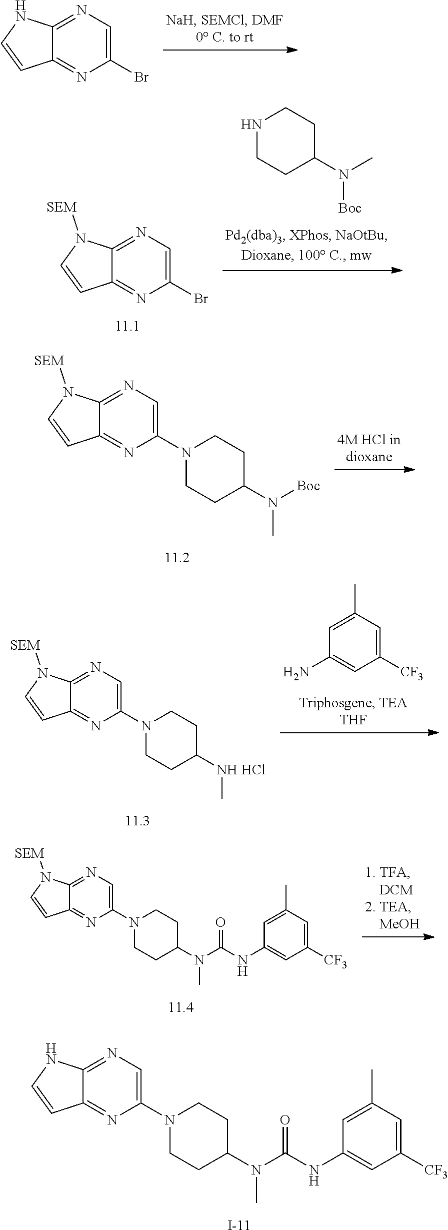

Synthesis of compound 11.1. To a solution of 2-bromo-5H-pyrrolo[2,3-b]pyrazine (1 g, 5 mmol, 1.0 equiv) in DMF (10 mL) was added sodium hydride (0.728 g, 15 mmol, 3 equiv) at 0° C. and stirred for 45 min. To the mixture was added 2-(trimethylsilyl)ethoxymethyl chloride (1.25 g, 7.5 mmol, 1.5 equiv) dropwise and stirred at room temperature for 1 h. The reaction mixture was quenched with cold water carefully and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford 11.1 (1 g, 60%). MS (ES): m/z 328.29 [M+H]⁺.

Synthesis of compound 11.2. To a solution of 11.1 (1 g, 3.1 mmol, 1 equiv) and tert-butyl methyl(piperidin-4-yl)carbamate in dioxane (10 mL) was added sodium tert-butoxide (0.878 g, 9.1 mmol, 3 equiv) and degassed by bubbling through a stream of argon for 10 min. To the mixture was added 2-dicyclohexylphosphino-2′,4′,6′-triisopropylbiphenyl (0.291 g, 0.6 mmol, 0.2 equiv) and tris(dibenzylideneacetone)dipalladium(O) (0.30 g, 0.3 mmol, 0.1 equiv) and degassed for another 5 min. The reaction mixture was stirred at 100° C. for 30 min in a microwave reactor. It was cooled to room temperature, quenched with water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 13% ethyl acetate in hexane) to afford 11.2 (0.8 g, 57%). MS (ES): m/z 462.20 [M+H]⁺.

Synthesis of compound 11.3. Compound 11.2 (0.3 g, 0.64 mmol, 1 equiv) was stirred with 4 M hydrochloric acid in dioxane (5 mL) at room temperature for 1.5 h. It was concentrated under reduced pressure to get 11.3 (0.23 g, 98%). It was used in the next step without purification. MS(ES): m/z 362.37 [M+H]⁺.

Synthesis of compound 11.4. Compound 11.4 was prepared from compound 11.3 and 3-methyl-5-(trifluoromethyl)aniline, following the procedure described in the synthesis of I-9. The product was purified by flash column chromatography on silica gel (CombiFlash®, 2% methanol in DCM). MS(ES): m/z 563.43 [M+H]⁺.

Synthesis of compound I-11. To a stirred solution of 11.4 (0.12 g, 0.213 mmol, 1 equiv) in DCM (5 mL) at 0° C. was added trifluoroacetic acid (1.5 mL). The reaction mixture was allowed to warm to room temperature and stirred for 2 h. It was diluted with DCM and quenched with a saturated aqueous solution of sodium bicarbonate. The organic layer was separated and washed with brine, dried over anhydrous sodium sulfate, filtered, and concentrated under reduced pressure. The residue was dissolved in methanol (5 mL) and water (2 mL) and triethylamine (0.215 g, 0.2 mmol, 10 equiv) was added. The mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.2% methanol in DCM) to afford I-11 (0.04 g, 43%). MS(ES): m/z 433.28 [M+H]⁺; ¹H NMR (DMSO-d₆, 400 MHz): 8.54 (s, 1H), 8.07 (s, 1H), 7.76 (s, 1H), 7.59 (d, J=7.2 Hz, 10.4 Hz, 2H), 7.10 (s, 1H), 6.33 (s, 1H), 4.40-4.30 (m, 3H), 2.91-2.80 (m, 2H), 2.68 (s, 3H), 2.34 (s, 3H), 1.79-1.73 (m, 2H), 1.67-1.64 (m, 2H).

The following intermediates were prepared following the procedure of I-11:

| Cmpd. | | |
|---|---|---|
| No. | Structure and Name | Analytical Data |
| I-38 | 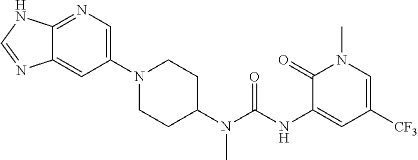 [SMILES: CN(C(=O)Nc1cc(C(F)(F)F)cn(C)c1=O)C1CCN(c2cnc3[nH]cnc3c2)CC1] | MS (ES): m/z 450.39 [M + H]⁺ ¹H-NMR (400 MHz, DMSO-d₆): δ 12.82 (s, 1H), 12.36 (s, 1H), 8.25 (s, 1H), 8.17 (s, 1H), 8.04 (s, 1H), 7.91 (s, 1H), 7.62 (s, 1H), 4.25- 4.13 (m, 1H), 3.65-3.77 (m, 2H), 3.58 (s, 3H), 2.89 (s, 3H), 2.82- 2.76 (m, 2H), 1.91-1.89 (m, 2H), 1.69-1.67 (m, 2H) |
| | 1-(1-(3H-imidazo[4,5-b]pyridin-6-yl)piperidin-4- | |
| | yl)-1-methyl-3-(1-methyl-2-oxo-5- | |
| | (trifluoromethyl)-1,2-dihydropyridin-3-yl)urea | |
| I-39 | 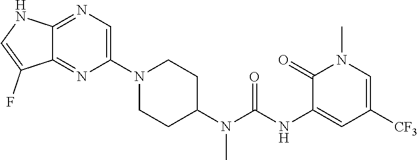 [SMILES: CN(C(=O)Nc1cc(C(F)(F)F)cn(C)c1=O)C1CCN(c2cnc3[nH]cc(F)c3n2)CC1] | MS (ES): m/z 468.30 [M + H]⁺ ¹H-NMR (400 MHz, DMSO-d₆): δ 11.40 (s, 1H), 8.17 (s, 2H), 8.03 (s, 1H), 7.90 (s, 1H), 7.58 (s, 1H), 4.44- 4.41 (d, J = 12.0 Hz, 2H), 4.30- 4.27 (m, 1H), 3.59 (s, 3H), 2.94- 2.88 (m, 2H), 2.84 (s, 3H), 1.77- 1.72 (m, 2H), 1.68-1.65 (m, 2H) |
| | 1-(1-(7-fluoro-5H-pyrrolo[2,3-b]pyrazin-2- | |
| | yl)piperidin-4-yl)-1-methyl-3-(1-methyl-2-oxo-5- | |
| | (trifluoromethyl)-1,2-dihydropyridin-3-yl)urea | |
| I-40 | 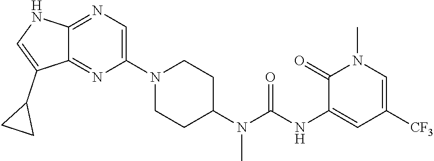 [SMILES: CN(C(=O)Nc1cc(C(F)(F)F)cn(C)c1=O)C1CCN(c2cnc3[nH]cc(C4CC4)c3n2)CC1] | MS (ES): m/z 490.33 [M + H]⁺ ¹H-NMR (400 MHz, DMSO-d₆): δ 11.19 (s, 1H), 8.17 (s, 1H), 8.03 (s, 1H), 8.00 (s, 1H), 7.90 (s, 1H), 7.31 (s, 1H), 4.38-4.35 (m, 2H), 4.51- 4.22 (m, 1H), 3.58 (s, 3H), 2.88- 2.82 (m, 1H), 2.84 (s, 3H), 1.97- 1.89 (m, 1H), 1.81-1.71 (m, 2H), 1.70-1.63 (m, 2H), 1.33-1.23 (s, 1H), 0.88-0.81 (m, 4H) |
| | 1-(1-(7-cyclopropyl-5H-pyrrolo[2,3-b]pyrazin-2- | |
| | yl)piperidin-4-yl)-1-methyl-3-(1-methyl-2-oxo-5- | |
| | (trifluoromethyl)-1,2-dihydropyridin-3-yl)urea | |
| I-47 | 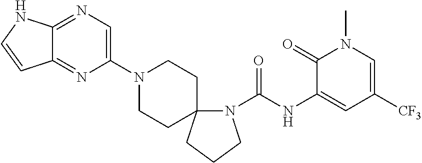 [SMILES: Cn1cc(C(F)(F)F)cc(NC(=O)N2CCCC23CCN(c2cnc4[nH]ccc4n2)CC3)c1=O] N-(1-methyl-2-oxo-5-(trifluoromethyl)-1,2- dihydropyridin-3-yl)-8-(5H-pyrrolo[2,3- | MS (ES): m/z 476.44 [M + H]⁺ ¹H NMR (DMSO-d₆, 400 MHz): δ 11.52 (s, 1H), 8.05-8.04 (d, J = 2.0 Hz, 1H), 8.03 (s, 1H), 7.98 (s, 1H), 7.59 (s, 1H), 7.56-7.55 (m, 1H), 6.31-6.29 (m, 1H), 4.28-4.25 (d, J = 11.6 Hz, 1H), 3.56 (s, 3H), 3.55- 3.51 (m, 1H), 3.38-3.37 (m, 1H), 2.90-2.79 (m, 4H), 2.06-2.02 (m, 2H), 1.94-1.89 (m, 2H), 1.43- 1.40 (d, J = 11.2 Hz, 2H), 1.10- 1.07 (t, J = 6.8 Hz, 1H) |
| | b]pyrazin-2-yl)-1,8-diazaspiro[4.5]decane-1- | |
| | carboxamide | |
| I-52 | 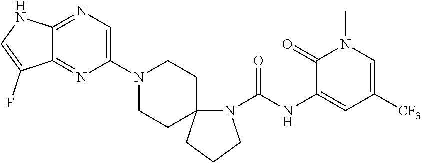 [SMILES: Cn1cc(C(F)(F)F)cc(NC(=O)N2CCCC23CCN(c2cnc4[nH]cc(F)c4n2)CC3)c1=O] | MS (ES): m/z 493.44 [M + H]⁺ ¹H NMR (DMSO-d₆, 400 MHz): δ 8.31 (s, 1H), 8.15 (s, 1H), 7.99 (s, 1H), 7.89 (s, 1H), 7.63 (s, 1H), 4.15- 4.13 (m, 1H), 3.63-3.58 (m, 4H), 2.96-2.90 (m, 4H), 2.90 (s, 3H), 2.80-2.74 (m, 2H), 1.92-1.90 (m, 2H), 1.69-1.67 (m, 2H) |
| | 8-(7-fluoro-5H-pyrrolo[2,3-b]pyrazin-2-yl)-N-(1- | |
| | methyl-2-oxo-5-(trifluoromethyl)-1,2- | |
| | dihydropyridin-3-yl)-1,8-diazaspiro[4.5]decane- | |
| | 1-carboxamide | |
| I-68 | 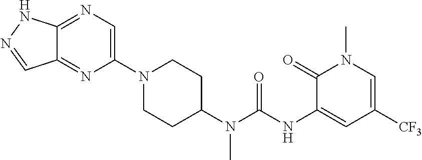 [SMILES: CN(C(=O)Nc1cc(C(F)(F)F)cn(C)c1=O)C1CCN(c2cnc3[nH]ncc3n2)CC1] | MS (ES): m/z 451.29 [M + H]+ 1H NMR (DMSO-d6, 400 MHz): δ 13.55 (s, 1H), 8.50 (s, 1H), 8.17- 8.16 (d, J = 2.4 Hz, 1H), 8.04-8.03 (d, J = 1.2 Hz, 1H), 7.99-7.98 (d, J = 1.2 Hz, 1H) 7.89 (s, 1H), 4.49- 4.46 (d, J = 13.2 Hz, 2H), 4.36-4.27 (m, 1H), 3.58 (s, 3H), 2.99-2.93 (m, 2H), 2.82 (s, 3H), 1.77-1.71 (m, 2H), 1.67-1.64 (m, 2H) |
| | 1-(1-(1H-pyrazolo[3,4-b]pyrazin-5-yl)piperidin- | |
| | 4-yl)-1-methyl-3-(1-methyl-2-oxo-5- | |
| | (trifluoromethyl)-1,2-dihydropyridin-3-yl)urea | |
| I-58-a and I- 58-b^{d} | 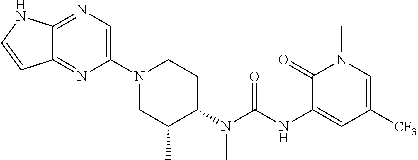 [SMILES: C[C@@H]1CN(c2cnc3[nH]ccc3n2)CC[C@@H]1N(C)C(=O)Nc1cc(C(F)(F)F)cn(C)c1=O] 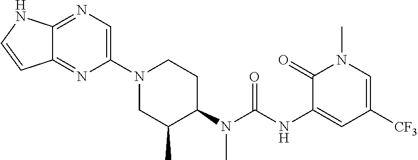 [SMILES: C[C@H]1CN(c2cnc3[nH]ccc3n2)CC[C@H]1N(C)C(=O)Nc1cc(C(F)(F)F)cn(C)c1=O] | MS (ES): m/z 464.37 [M + H]⁺ ¹H-NMR (400 MHz, DMSO-d₆): δ 11.53 (s, 1H), 8.20-8.19 (d, J = 2.4 Hz, 1H), 8.03 (s, 2H), 7.92 (s, 1H), 7.57-7.55 (t, J = 3.2 Hz, 1H), 6.31- 6.29 (m, 1H), 4.43-4.40 (m, 1H), 4.27-4.24 (m, 1H), 4.15-4.12 (m, 1H), 3.59 (s, 3H), 3.03-3.00 (m, 1H), 2.99 (s, 3H), 2.89-2.79 (m, 1H), 2.30-2.22 (m, 1H), 2.19-2.11 (m, 1H), 1.63-1.60 (m, 1H), 0.98- 0.96 (d, J = 6.8 Hz, 3H). |
| | 1-methyl-1-((3R,4S)-3-methyl-1-(5H-pyrrolo[2,3- | |
| | b]pyrazin-2-yl)piperidin-4-yl)-3-(1-methyl-2- | |
| | oxo-5-(trifluoromethyl)-1,2-dihydropyridin-3- | |
| | yl)urea; | |
| | 1-methyl-1-((3S,4R)-3-methyl-1-(5H-pyrrolo[2,3- | |
| | b]pyrazin-2-yl)piperidin-4-yl)-3-(1-methyl-2- | |
| | oxo-5-(trifluoromethyl)-1,2-dihydropyridin-3- | |
| | yl)urea | |
| I-70-a and I- 70-b^{b} | 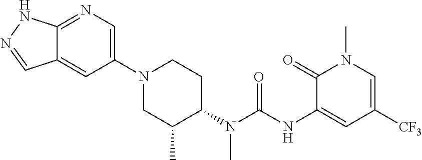 [SMILES: C[C@@H]1CN(c2cnc3[nH]ncc3c2)CC[C@@H]1N(C)C(=O)Nc1cc(C(F)(F)F)cn(C)c1=O] 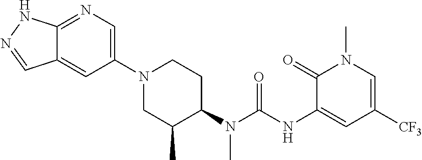 [SMILES: C[C@H]1CN(c2cnc3[nH]ncc3c2)CC[C@H]1N(C)C(=O)Nc1cc(C(F)(F)F)cn(C)c1=O] | MS (ES): m/z 464.30 [M + H]⁺ ¹H NMR (DMSO-d₆, 400 MHz): δ 13.38 (s, 1H), 8.42-8.41 (d, J = 2.4 Hz, 1H), 8.10-8.19 (d, J = 2.4 Hz, 1H), 8.04-8.03 (d, J = 1.2 Hz, 1H), 7.96-7.33 (m, 2H), 7.61-7.60 (d, J = 2.4 Hz, 1H), 4.19-4.17 (m, 1H), 3.65-3.59 (m, 1H), 3.6 (s, 3H), 3.37-3.36 (d, J = 1.2 Hz, 1H), 2.98 (s, 3H), 2.91-2.89 (m, 1H), 2.79- 2.74 (m, 1H), 2.46-2.25 (m, 2H), 1.66-1.64 (m, 1H), 1.33-1.25 (d, J = 7.2 Hz, 3H) |
| | 1-methyl-1-((3R,4S)-3-methyl-1-(1H- | |
| | pyrazolo[3,4-b]pyridin-5-yl)piperidin-4-yl)-3-(1- | |
| | methyl-2-oxo-5-(trifluoromethyl)-1,2- | |
| | dihydropyridin-3-yl)urea; | |
| | 1-methyl-1-((3S,4R)-3-methyl-1-(1H- | |
| | pyrazolo[3,4-b]pyridin-5-yl)piperidin-4-yl)-3-(1- | |
| | methyl-2-oxo-5-(trifluoromethyl)-1,2- | |
| | dihydropyridin-3-yl)urea | |
| I-73 | 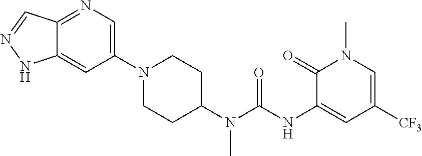 [SMILES: CN(C(=O)Nc1cc(C(F)(F)F)cn(C)c1=O)C1CCN(c2cnc3cn[nH]c3c2)CC1] 1-(1-(1H-pyrazolo[4,3-b]pyridin-6-yl)piperidin-4- | MS (ES): m/z 450.32 [M + H]⁺ ¹H-NMR (400 MHz, DMSO-d₆): δ 12.86 (s, 1H), 8.45-8.44 (d, J = 2.4 Hz, 1H), 8.18-8.17 (d, J = 2.4 Hz, 1H), 8.06 (s, 1H), 8.03 (s, 1H), 7.91 (s, 1H), 7.21-7.20 (d, J = 1.6 Hz, 1H), 4.26-4.19 (m, 1H), 3.89-3.86 (m, 2H), 3.58(s, 3H), 2.88 (s, 3H), 2.84-2.81 (m, 2H), 1.92-1.84 (m, 2H), 1.70-1.67 (m, 2H) |
| | yl)-1-methyl-3-(1-methyl-2-oxo-5- | |
| | (trifluoromethyl)-1,2-dihydropyridin-3-yl)urea | |
| I-74-a and I- 74-b^{d} | 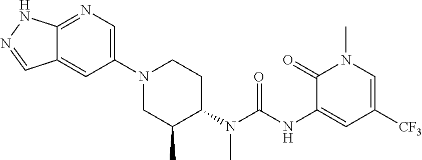 [SMILES: C[C@H]1CN(c2cnc3[nH]ncc3c2)CC[C@@H]1N(C)C(=O)Nc1cc(C(F)(F)F)cn(C)c1=O] | MS (ES): m/z 464.31 [M + H]⁺ ¹H NMR (DMSO-d₆, 400 MHz): δ 13.37 (s, 1H), 8.44 (s, 1H), 8.16 (s, 1H), 8.02 (s, 1H), 7.94 (s, 1H), 7.92 (s, 1H), 7.61 (s, 1H), 3.69-3.66 (m, 2H), 3.57 (s, 3H), 2.93-2.2.89 (m, 2H), 2.82-2.76 (m, 1H), 2.28 (s, 3H), 2.03-1.82 (m, 2H), 1.65-1.62 (m, 1H), 0.83-0.82 (d, J = 6.4 Hz, 3H |
| | 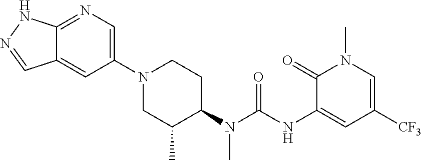 [SMILES: C[C@@H]1CN(c2cnc3[nH]ncc3c2)CC[C@H]1N(C)C(=O)Nc1cc(C(F)(F)F)cn(C)c1=O] | |
| | 1-methyl-1-((3S,4S)-3-methyl-1-(1H- | |
| | pyrazolo[3,4-b]pyridin-5-yl)piperidin-4-yl)-3-(1- | |
| | methyl-2-oxo-5-(trifluoromethyl)-1,2- | |
| | dihydropyridin-3-yl)urea; | |
| | 1-methyl-1-((3R,4R)-3-methyl-1-(1H- | |
| | pyrazolo[3,4-b]pyridin-5-yl)piperidin-4-yl)-3-(1- | |
| | methyl-2-oxo-5-(trifluoromethyl)-1,2- | |
| | dihydropyridin-3-yl)urea | |
| I-77 | 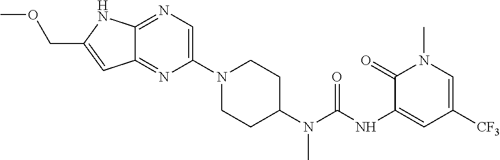 [SMILES: COCc1cc2nc(N3CCC(N(C)C(=O)Nc4cc(C(F)(F)F)cn(C)c4=O)CC3)cnc2[nH]1] | MS (ES): m/z 494.28 [M + H]⁺ ¹H NMR (DMSO-d₆, 400 MHz): δ 11.65 (s, 1H), 8.18 (s 1H), 8.03 (s, 1H), 8.02 (s, 1H), 7.90 (s, 1H), 6,29 (s, 1H), 4.51 (s, 2H), 4.39-4.35 (m, 2H), 4.34-4.26 (m, 1H), 3.59 (s, 3H), 3.32 (s, 3H), 2.89-2.86 (m, 2H), 2.84 (s, 3H), 1.78-1.72 (m, 2H), 1.67-1.64 (m, 2H) |
| | 1-(1-(6-(methoxymethyl)-5H-pyrrolo[2,3- | |
| | b]pyrazin-2-yl)piperidin-4-yl)-1-methyl-3-(1- | |
| | methyl-2-oxo-5-(trifluoromethyl)-1,2- | |
| | dihydropyridin-3-yl)urea | |
| I-79 | 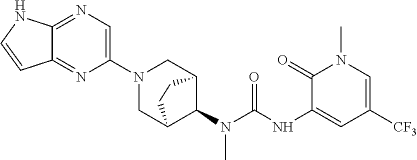 [SMILES: CN(C(=O)Nc1cc(C(F)(F)F)cn(C)c1=O)[C@H]1[C@@H]2CC[C@H]1CN(c1cnc3[nH]ccc3n1)C2] | MS (ES): m/z 476.19 [M + H]⁺ ¹H-NMR (400 MHz, DMSO-d₆): δ 11.43 (s, 1H), 8.15-8.11 (d, J = 17.2 Hz, 2H), 7.99 (s, 1H), 7.89 (s, 1H), 7.53 (s, 1H), 6.27 (s, 1H), 4.04 (m, 1H), 3.71 (m, 1H), 3.55 (s, 3H), 3.45 (m, 2H), 3.10 (s, 3H), 2.92 (m, 2H), 2.42-2.31 (m, 2H), 1.79 (m, 2H), 1.64 (m, 1H) |
| | 1-(3-(5H-pyrrolo[2,3-b]pyrazin-2-yl)-3- | |
| | azabicyclo[3.2.1]octan-8-yl)-1-methyl-3-(1- | |
| | methyl-2-oxo-5-(trifluoromethyl)-1,2- | |
| | dihydropyridin-3-yl)urea | |
| I-80 | 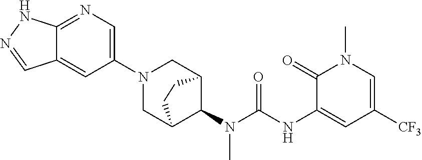 [SMILES: CN(C(=O)Nc1cc(C(F)(F)F)cn(C)c1=O)[C@H]1[C@@H]2CC[C@H]1CN(c1cnc3[nH]ncc3c1)C2] | MS (ES): m/z 476.22 [M + H]⁺ ¹H-NMR (400 MHz, DMSO-d₆): δ 13.24 (s, 1H), 8.42 (s, 1H), 8.15 (s, 1H), 8.10 (s, 1H), 8.00 (s, 1H), 7.89 (s, 1H), 7.44 (s, 1H), 3.55 (s, 3H), 3.50-3.42 (m, 2H), 3.33-3.29 (m, 1H), 3.08 (s, 3H), 2.95-2.92 (m, 4H), 1.77-1.75 (m, 4H). |
| | 1-(3-(1H-pyrazolo[3,4-b]pyridin-5-yl)-3- | |
| | azabicyclo[3.2.1]octan-8-yl)-1-methyl-3-(1- | |
| | methyl-2-oxo-5-(trifluoromethyl)-1,2- | |
| | dihydropyridin-3-yl)urea | |
| I-111-a and I- 111-b^{d} | 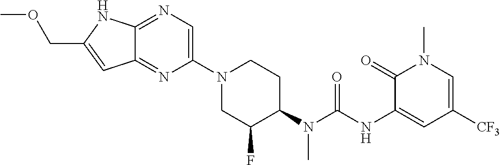 [SMILES: COCc1cc2nc(N3CC[C@@H](N(C)C(=O)Nc4cc(C(F)(F)F)cn(C)c4=O)[C@@H](F)C3)cnc2[nH]1] | MS (ES): m/z 512.34 [M + H]⁺ ¹H-NMR (400 MHz, DMSO-d₆): δ 11.65 (s, 1H), 8.18 (s, 1H), 8.05 (s, 1H), 8.02 (s, 1H), 7.97 (s, 1H), 6.27 (s, 1H), 5.00-4.87 (d, JHF = 50.4 Hz, 1H), 4.59-4.4.40 (m, 4H), 4.15 (s, 2H), 3.59 (s, 3H), 3.29 (s, 3H), 2.96 (s, 3H), 2.69-2.67 (s, 1H), 2.24-2.21 (m, 1H), 1.68-1.62 (m, 1H) |
| | 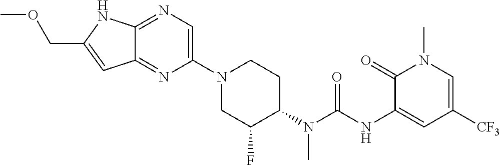 [SMILES: COCc1cc2nc(N3CC[C@H](N(C)C(=O)Nc4cc(C(F)(F)F)cn(C)c4=O)[C@H](F)C3)cnc2[nH]1] | |
| | 1-((3S,4R)-3-fluoro-1-(6-(methoxymethyl)-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea; | |
| | 1-((3R,4S)-3-fluoro-1-(6-(methoxymethyl)-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea | |
| I-112 | 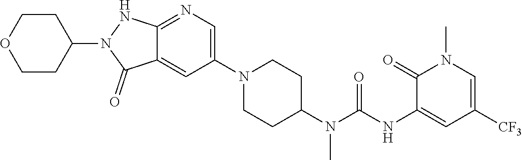 [SMILES: CN(C(=O)Nc1cc(C(F)(F)F)cn(C)c1=O)C1CCN(c2cnc3[nH]n(C4CCOCC4)c(=O)c3c2)CC1] | MS (ES): m/z 550.33 [M + H]⁺ ¹H NMR (DMSO-d₆, 400 MHz): 10.35 (s, 1H), 8.49 (s, 1H), 8.17 (s, 1H), 8.03 (s, 1H), 7.90 (s, 1H), 7.60 (s, 1H), 4.54-4.6 (m, 1H), 4.23-4.13 (m, 1H), 3.96-3.93 (m, 3H), 3.58 (s, 3H), 3.47-3.42 (m, 3H), 2.88 (s, 3H), 2.84-2.77 (m, 2H), 2.02-1.84 (m, 4H), 1.68-1.65 (m, 4H) |
| | 1-methyl-3-(1-methyl-2-oxo-5-(trifluoromethyl)- | |
| | 1,2-dihydropyridin-3-yl)-1-(1-(3-oxo-2- | |
| | (tetrahydro-2H-pyran-4-yl)-2,3-dihydro-1H- | |
| | pyrazolo[3,4-b]pyridin-5-yl)piperidin-4-yl)urea | |
| I-113-a and I- 113-b^{e} | 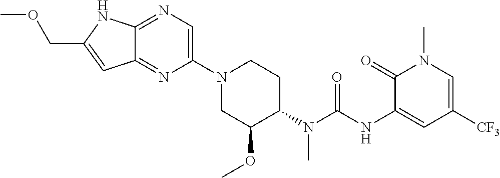 [SMILES: COCc1cc2nc(N3CC[C@H](N(C)C(=O)Nc4cc(C(F)(F)F)cn(C)c4=O)[C@@H](OC)C3)cnc2[nH]1] | MS (ES): m/z 523.23 [M + H]⁺ ¹H NMR (DMSO-d₆, 400 MHz): δ 11.69 (s, 1H), 8.09 (s, 2H), 8.01 (s, 2H), 6.30 (s, 1H), 4.71-4.69 (m, 1H), 4.51 (s, 2H), 4.31-4.28 (m, 1H), 3.58 (s, 3H), 3.44 (s, 3H), 3.42-3.34 (m, 4H), 3.29 (s, 3H), 2.88 (s, 3H), 1.82-1.75 (m, 2H) |
| | 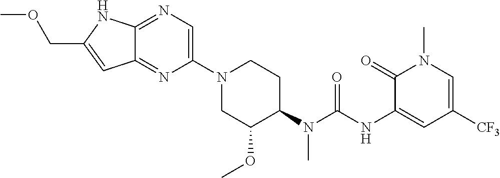 [SMILES: COCc1cc2nc(N3CC[C@@H](N(C)C(=O)Nc4cc(C(F)(F)F)cn(C)c4=O)[C@H](OC)C3)cnc2[nH]1] | |
| | 1-((3S,4S)-3-methoxy-1-(6-(methoxymethyl)-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea; | |
| | 1-((3R,4R)-3-methoxy-1-(6-(methoxymethyl)- | |
| | 5H-pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea | |
| I-115-a and I- 115-b^{c} | 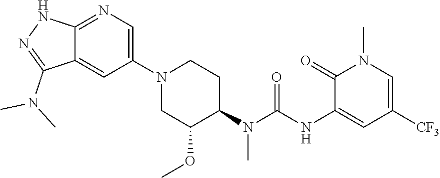 [SMILES: CO[C@@H]1CN(c2cnc3[nH]nc(N(C)C)c3c2)CC[C@H]1N(C)C(=O)Nc1cc(C(F)(F)F)cn(C)c1=O] | MS (ES): m/z 523.45 [M + H]⁺ ¹H NMR (DMSO-d₆, 400 MHz): δ 12.19 (s, 1H), 8.37-8.36 (d, J = 2.4 Hz, 1H), 8.31 (s, 1H), 8.08 (s, 1H), 8.01 (s, 1H), 7.71-7.70 (d, J = 2.4 Hz, 1H), 3.94-3.92 (m, 1H), 3.58 (s, 3H), 3.55-3.53 (m, 2H), 3.37 (s, 3H), 2.97 (s, 6H), 2.94-2.90 (m, 2H), 2.92 (s, 3H), 2.79-2.71 (m, 1H), 2.02-1.93 (m, 1H), 1.78-1.71 (s, 1H) |
| | 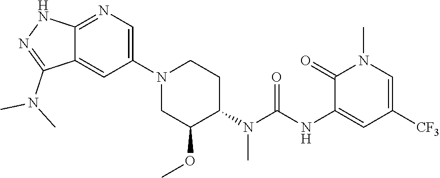 [SMILES: CO[C@H]1CN(c2cnc3[nH]nc(N(C)C)c3c2)CC[C@@H]1N(C)C(=O)Nc1cc(C(F)(F)F)cn(C)c1=O] | |
| | 1-((3R,4R)-1-(3-(dimethylamino)-1/- | |
| | pyrazolo[3,4-b]pyridin-5-yl)-3-methoxypiperidin- | |
| | 4-yl)-1-methyl-3-(1-methyl-2-oxo-5- | |
| | (trifluoromethyl)-1,2-dihydropyridin-3-yl)urea; | |
| | 1-((3S,4S)-1-(3-(dimethylamino)-1H- | |
| | pyrazolo[3,4-b]pyridin-5-yl)-3-methoxypiperidin- | |
| | 4-yl)-1-methyl-3-(1-methyl-2-oxo-5- | |
| | (trifluoromethyl)-1,2-dihydropyridin-3-yl)urea | |
| I-117-a and I- 117-b^{f} | 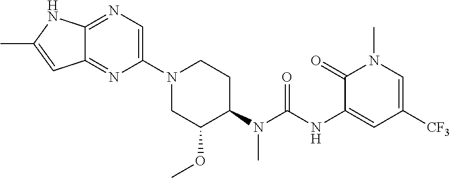 [SMILES: CO[C@@H]1CN(c2cnc3[nH]c(C)cc3n2)CC[C@H]1N(C)C(=O)Nc1cc(C(F)(F)F)cn(C)c1=O] | MS (ES): m/z 494.25 [M + H]⁺ ¹H-NMR (400 MHz, DMSO-d₆): δ 11.41 (s, 1H), 8.08 (s, 1H), 8.03 (s, 1H), 8.01 (s, 1H), 7.95 (s, 1H), 6.07 (s, 1H), 4.68-4.66 (m, 1H), 4.27- 4.24 (m, 1H), 3.58 (s, 3H), 3.43- 3.42 (m, 3H), 3.28 (s, 3H), 2.84 (s, 3H), 2.67-2.65 (m, 1H), 2.39 (s, 3H), 1.84-1.17 (m 2H) |
| | 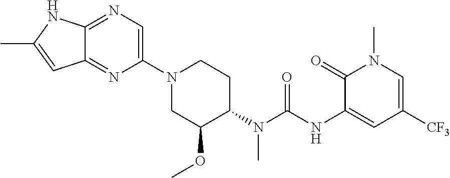 [SMILES: CO[C@H]1CN(c2cnc3[nH]c(C)cc3n2)CC[C@@H]1N(C)C(=O)Nc1cc(C(F)(F)F)cn(C)c1=O] | |
| | 1-((3R,4R)-3-methoxy-1-(6-methyl-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea; | |
| | 1-((3S,4S)-3-methoxy-1-(6-methyl-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea | |
| I-118-a and I- 118-b^{c} | 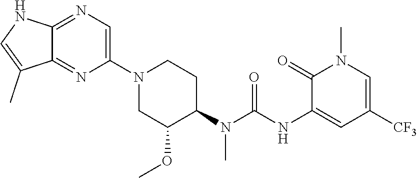 [SMILES: CO[C@@H]1CN(c2cnc3[nH]cc(C)c3n2)CC[C@H]1N(C)C(=O)Nc1cc(C(F)(F)F)cn(C)c1=O] | MS (ES): m/z 494.11 [M + H]⁺ ¹H-NMR (400 MHz, DMSO-d₆): δ 11.24 (s, 1H), 8.10 (s, 1H), 8.11 (s, 1H), 8.01 (s, 1H), 7.38 (s, 1H), 7.38 (s, 1H), 4.74-4.72 (d, J = 8.0Hz, 1H), 3.34-3.33 (d, J = 12.0Hz, 1H), 4.02-4.01 (d, J = 8.0Hz, 2H), 3.58 (s, 3H), 3.47 (s, 3H), 3.85 (s, 3H), 3.20 (s, 3H), 1.90-1.76 (m, 2H), 1.21-1.13 (m, 2H) |
| | 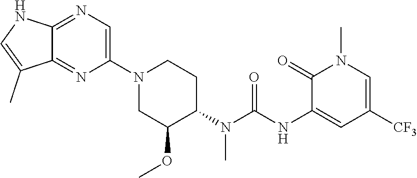 [SMILES: CO[C@H]1CN(c2cnc3[nH]cc(C)c3n2)CC[C@@H]1N(C)C(=O)Nc1cc(C(F)(F)F)cn(C)c1=O] | |
| | 1-((3R,4R)-3-methoxy-1-(7-methyl-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea; | |
| | 1-((3S,4S)-3-methoxy-1-(7-methyl-5H- | |
| | pyrrolo[2,3-b]pyrazin-2-yl)piperidin-4-yl)-1- | |
| | methyl-3-(1-methyl-2-oxo-5-(trifluoromethyl)- | |
| | 1,2-dihydropyridin-3-yl)urea | |
| I-159-a and I- 159-b^{d} | 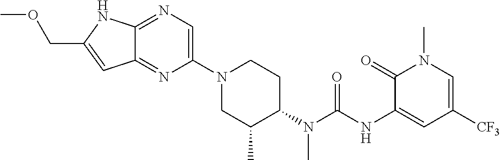 [SMILES: COCc1cc2nc(N3CC[C@H](N(C)C(=O)Nc4cc(C(F)(F)F)cn(C)c4=O)[C@H](C)C3)cnc2[nH]1] 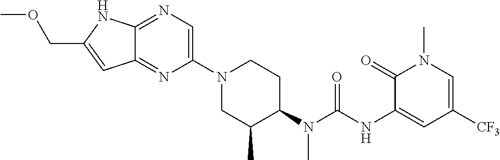 [SMILES: COCc1cc2nc(N3CC[C@@H](N(C)C(=O)Nc4cc(C(F)(F)F)cn(C)c4=O)[C@@H](C)C3)cnc2[nH]1] | MS (ES): m/z 508.28 [M + H]⁺ ¹H NMR (DMSO-d₆, 400 MHz): δ 11.63 (s, 1H), 8.20 (d, J = 2.4 Hz, 1H), 8.04 (d, J = 1.2 Hz, 1H), 8.01 (s, 1H), 7.93 (s, 1H), 6.27 (d, J = 2.0 Hz, 1H), 4.51 (s, 2H), 4.42-4.39 (d, J = 12.4 Hz, 1H), 4.28-4.25 (d, J = 12.0 Hz, 1H), 4.14-4.11 (d, J = 12.8 Hz, 1H), 3.60 (s, 3H), 3.34 (s, 3H), 3.03-2.99 (d, J = 15.6 Hz, 1H), 2.95 (s, 3H), 2.86-2.80 (t, J = 10.4 Hz, 1H), 2.27-2.09 (m, 2H), 1.63- 1.61 (d, J = 9.6 Hz, 1H), 0.98-0.97 (d, J = 7.2 Hz, 1H) |
| | 1-((3R,4S)-1-(6-(methoxymethyl)-5H- | |
| | pyrrolo[2,3-b]pyrazin-2-yl)-3-methylpiperidin-4- | |
| | yl)-1-methyl-3-(1-methyl-2-oxo-5- | |
| | (trifluoromethyl)-1,2-dihydropyridin-3-yl)urea; | |
| | 1-((3S,4R)-1-(6-(methoxymethyl)-5H- | |
| | pyrrolo[2,3-b]pyrazin-2-yl)-3-methylpiperidin-4- | |
| | yl)-1-methyl-3-(1-methyl-2-oxo-5- | |
| | (trifluoromethyl)-1,2-dihydropyridin-3-yl)urea | |

^{a}Separated by chiral SFC (Method A);
^{b}Separated by chiral SFC (Method B);
^{c}Separated by chiral HPLC (Method D);
^{d}Separated by chiral SFC (Method H);
^{e}Separated by chiral SFC (Method F);
^{f}Separated by chiral SFC (Method I).

### Example 12: N-(5-(4-(1-methyl-3-(3-methyl-5-(trifluoromethyl)phenyl)ureido)piperidin-1-yl)pyridin-2-yl)acetamide

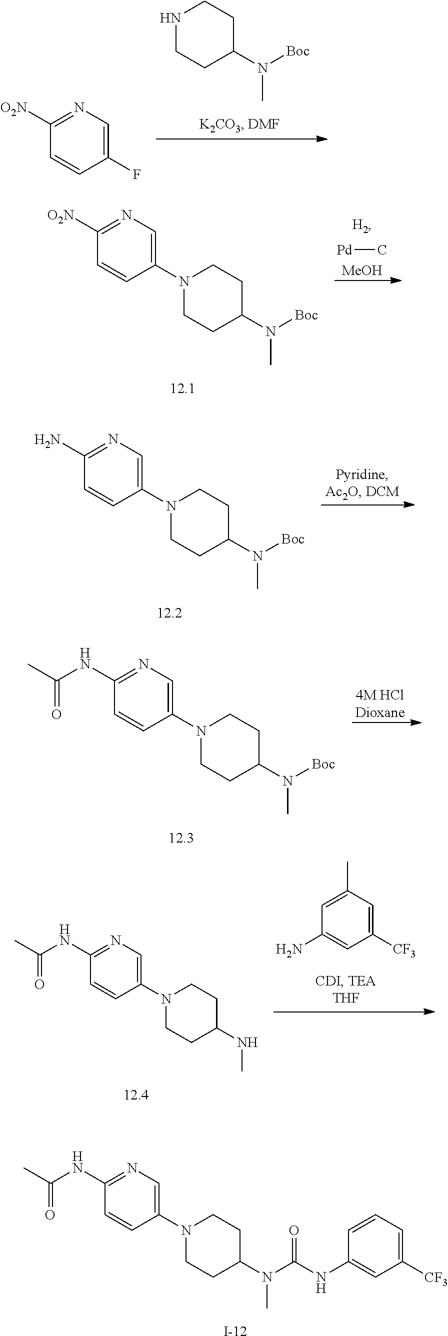

Synthesis of compound 12.1. A mixture of 5-fluoro-2-nitropyridine (0.500 g, 3.52 mmol, 1.0 equiv), tert-butyl methyl(piperidin-4-yl)carbamate (0.754 g, 3.52 mmol, 1.0 equiv), and potassium carbonate (1.457 g, 10.56 mmol, 3.0 equiv) in DMF (5 mL) was stirred at room temperature for 2 h. It was transferred into ice-water. The precipitated solids were collected by filtration and dried under reduce pressure to afford 12.1 (0.750 g, 63%). MS(ES): m/z 337.22 [M+H]⁺.

Synthesis of compound 12.2. A mixture of compound 12.1 (0.750 g, 2.23 mmol, 1.0 equiv) and 10% palladium on carbon (0.375 g) in methanol (5 mL) was stirred under hydrogen (1 atm) for 1 h. The mixture was filtered through a pad of Celite® and rinsed with methanol. The filtrate was concentrated under reduced pressure to afford 12.2 (0.510 g, 75%). MS(ES): m/z 307.0 and 308.4 [M+H]⁺.

Synthesis of compound 12.3. To a solution of 12.2 (0.510 g, 1.66 mmol, 1.0 equiv) in DCM (5.0 mL) was added pyridine (0.67 mL, 8.32 mmol, 5.0 equiv), followed by acetic anhydride (0.42 mL, 4.16 mmol, 2.5 equiv) at room temperature and stirred for 16 h. The reaction mixture was poured in ice-water and extracted with DCM. The combined organic layers were washed with brine, dried over anhydrous sodium sulfate, filtered, and concentrated under reduced pressure. The residue was purified by flash column chromatography on silica gel (CombiFlash®, 3.1% methanol in DCM) to afford 12.3 (0.400 g, 69%). MS (ES): m/z 349.20 [M+H]⁺.

Synthesis of compound 12.4. To a cooled solution of 12.3 (0.400 g, 1.15 mmol, 1.0 equiv) in dioxane (1 mL) was added 4 M hydrochloric acid in dioxane (4.0 mL) dropwise at 0° C. The reaction mixture was stirred at room temperature for 30 min. It was transferred into saturated aqueous solution of sodium bicarbonate and extracted with DCM. The combined organic layers were washed with brine, dried over anhydrous sodium sulfate, filtered, and concentrated under reduced pressure to afford 12.4. MS(ES): m/z 249.19 [M+H]⁺.

Synthesis of compound I-12. To a solution of 12.4 (0.100 g, 0.403 mmol, 1.0 equiv), 3-methyl-5-(trifluoromethyl)aniline (0.070 g, 0.403 mmol, 1.0 equiv) and triethylamine (0.203 g, 2.01 mmol, 5.0 equiv) in THE (2 mL) was added 1,1′-carbonyldiimidazole (0.163 g, 1.00 mmol, 2.5 equiv) at 0° C. The reaction mixture was stirred at room temperature for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.3% methanol in DCM) to afford I-12 (0.025 g, 14%). MS(ES): m/z: 450.32 [M+H]⁺; ¹H NMR (DMSO-d₆, 400 MHz): δ 10.24 (s, 1H), 8.54 (s, 1H), 8.03 (d, J=2.8 Hz, 1H), 7.94 (d, J=8.8 Hz, 1H), 7.77 (s, 1H), 7.621 (s, 1H), 7.45 (dd, J=2.8 Hz, 1H), 7.11 (s, 1H), 4.21 (m, 1H), 3.76 (m, 2H), 2.86 (s, 3H), 2.76 (m, 2H), 2.35 (s, 3H), 2.05 (s, 3H), 1.88-1.80 (m, 2H), 1.66 (m, 2H).

### Example 13: N-(5-(4-(1-methyl-3-(3-methyl-5-(trifluoromethyl)phenyl)ureido)piperidin-1-yl)pyrazin-2-yl)acetamide

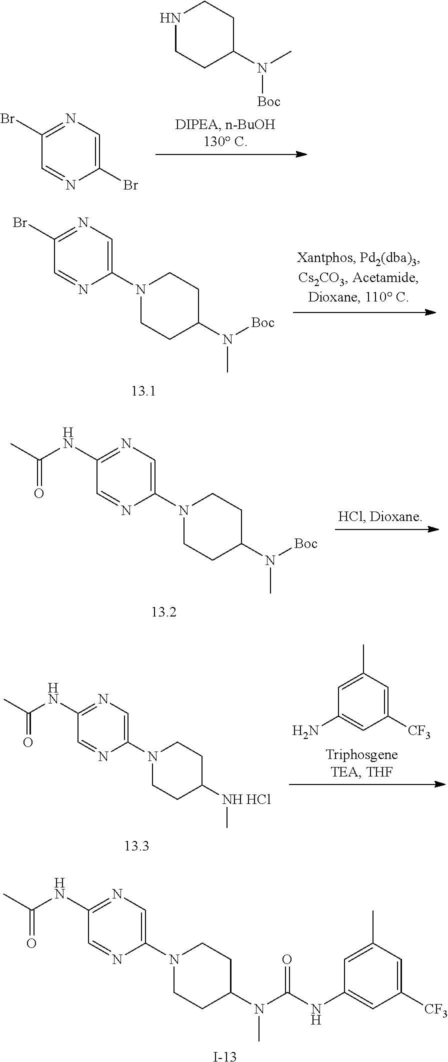

Synthesis of compound 13.1. A solution of 2,5-dibromopyrazine (2 g, 8.4 mmol, 1.0 equiv), tert-butyl methyl(piperidin-4-yl)carbamate (3.6 g, 16.8 mmol, 2.0 equiv), and DIPEA (1.35 g. 10.5 mmol, 5 equiv) in n-butanol (20 mL) was stirred at 130° C. for 30 min. It was cooled to room temperature, poured into cold water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford 13.1 (2 g, 64%). MS (ES): m/z 371.10 and 373.1 [M+H]⁺.

Synthesis of compound 13.2. A mixture of 13.1 (2 g, 2.6 mmol, 1 equiv) and acetamide (0.477 g, 8 mmol, 3.0 equiv) in dioxane (10 mL) was degassed by bubbling through a stream of argon for 5 min. Cesium carbonate (2.6 g, 8.0 mmol, 3 equiv), XantPhos (0.311 mg, 0.5 mmol, 0.2 equiv), and Pd₂(dba)₃ (0.246 g, 0.26 mmol, 0.1 equiv) were added and degassed for another 5 min. The reaction mixture was stirred at 100° C. for 90 min. It was cooled to room temperature, transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford 13.2 (0.8 g, 42.5%). MS (ES): m/z 350.2 [M+H]⁺.

Synthesis of compound 13.3. Compound 13.2 (0.8 g, 2.2 mmol, 1 equiv) was stirred with 4 M hydrogen chloride in dioxane (5 mL) at room temperature for 1.5 h. It was concentrated under reduced pressure to afford 13.3 (0.550 g, 96%). It was used in the next step without purification. MS(ES): m/z 250.12 [M+H]⁺.

Synthesis of compound I-13. Compound I-13 was prepared from compound 13.3 and 3-methyl-5-(trifluoromethyl)aniline, following the procedure described in the synthesis of I-9. The product was purified by flash column chromatography on silica gel (CombiFlash®, 2% methanol in DCM). MS(ES): m/z 451.28 [M+H]⁺; ¹H NMR (DMSO-d₆, 400 MHz): 10.30 (s, 1H), 8.77 (s, 1H), 8.53 (s, 1H), 8.12 (s, 1H), 7.76 (s, 1H), 7.60 (s, 1H), 7.10 (s, 1H), 4.38 (m, 2H), 4.30 (m, 1H), 2.91 (m, 2H), 2.80 (s, 3H), 2.34 (s, 3H), 2.06 (s, 3H), 1.80-1.60 (m, 4H).

### Example 14: N-(5-(4-(1-methyl-3-(1-methyl-2-oxo-5-(trifluoromethyl)-1,2-dihydropyridin-3-yl)ureido)piperidin-1-yl)pyrazin-2-yl)acetamide

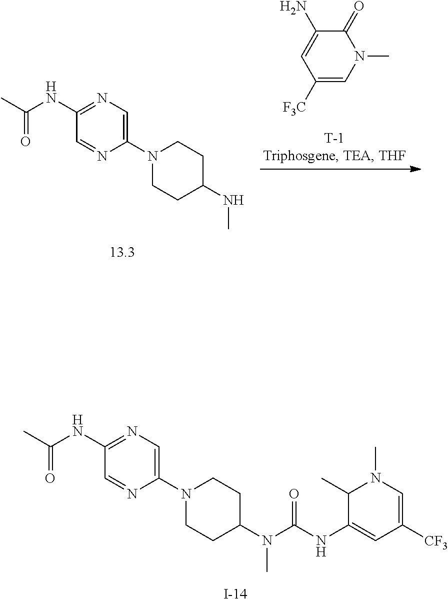

Synthesis of compound I-14. Compound I-14 was prepared from compound 13.3 and T-1, following the procedure described in the synthesis of 1-9. The product was purified by flash column chromatography on silica gel (CombiFlash®, 2% methanol in DCM). MS(ES): m/z 468.37 [M+H]⁺; ¹H NMR (DMSO-d₆, 400 MHz): 10.30 (s, 1H), 8.17 (s, 1H), 8.12 (s, 1H), 8.04 (s, 1H), 7.90 (s, 1H), 4.39 (m, 2H), 4.30 (m, 1H), 3.60 (s, 3H), 2.88 (m, 2H), 2.86 (s, 3H), 2.06 (s, 3H), 1.66 (m, 4H).

### Example 15: 1-(1-(5H-pyrrolo[2,3-b]pyrazin-2-yl)piperidin-4-yl)-1-methyl-3-(1-methyl-2-oxo-5-(trifluoromethyl)-1,2-dihydropyridin-3-yl)urea

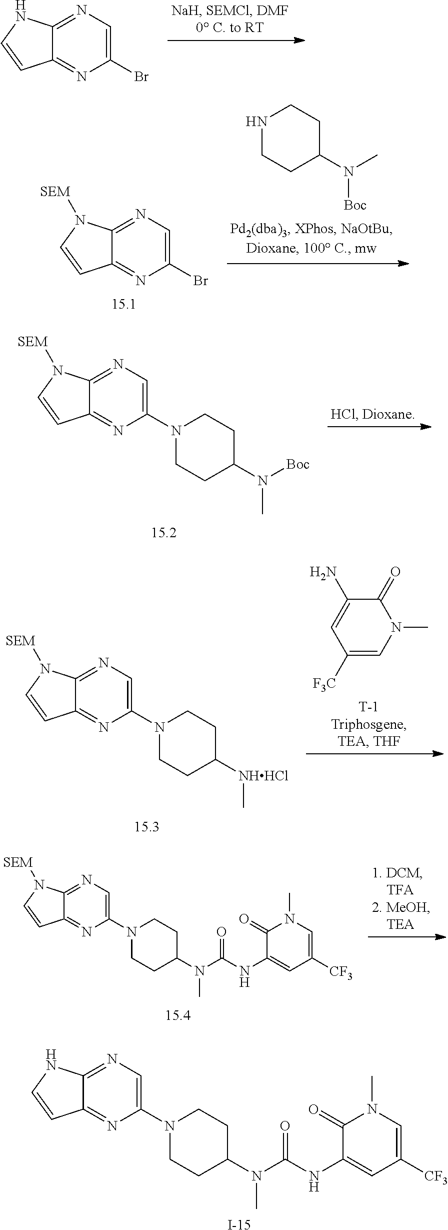

Synthesis of compound 15.1. To a solution of 2-bromo-5H-pyrrolo[2,3-b]pyrazine (1 g, 5.050 mmol, 1.0 equiv) in DMF (15 mL) was added sodium hydride (60% in mineral oil, 0.606 g, 15.15 mmol, 3.0 equiv) in small portions at 0° C. To the mixture was added SEM chloride (1.25 g, 75.75 mmol, 1.5 equiv) and stirred at room temperature for 1 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford 15.1 (1.2 g, 72%). MS(ES): m/z 329 [M+1].

Synthesis of compound 15.2. Compound 15.2 was prepared from compound 15.1 and tert-butyl methyl(piperidin-4-yl)carbamate, following the procedure described in the synthesis of 11.2. The product was purified by flash column chromatography on silica gel (CombiFlash®, 20% ethyl acetate in hexane). MS(ES): m/z 461 [M+H]⁺.

Synthesis of compound 15.3. Compound 15.2 (0.3 g, 0.650 mmol, 1.0 equiv) was stirred with 4 N HCl in dioxane (5 mL) at room temperature for 1 h. The reaction mixture was concentrated under reduced pressure to afford 15.3 (0.250 g). It was used in the next step without purification. MS(ES): m/z 361 [M+H]⁺.

Synthesis of compound 15.4. Compound 15.4 was prepared from compound 15.3 and T-1, following the procedure described in the synthesis of I-9. The product was purified by flash column chromatography on silica gel (CombiFlash®, 5% methanol in DCM). MS(ES): m/z 579 [M+1]⁺.

Synthesis of I-15. Compound I-15 was prepared from compound 15.4, following the procedure described in the synthesis of I-11. The product was purified by flash column chromatography on silica gel (CombiFlash®, 5% methanol in DCM). MS(ES): m/z 450.25 [M+H]⁺; ¹H NMR (DMSO-d₆, 400 MHz): δ 11.58 (s, 1H), 8.19 (s, 1H), 8.05 (s, 1H), 8.03 (s, 1H), 7.89 (s, 1H), 7.58 (s, 1H), 6.34 (s, 1H), 4.38 (d, J=12.4 Hz, 2H), 4.28 (m, 1H), 3.58 (s, 3H), 2.90-2.60 (m, 5H), 1.78-1.66 (m, 4H).

### Example 16: N-(5-(4-(1-methyl-3-(3-(trifluoromethyl)phenyl)ureido)piperidin-1-yl)pyrazin-2-yl)acetamide

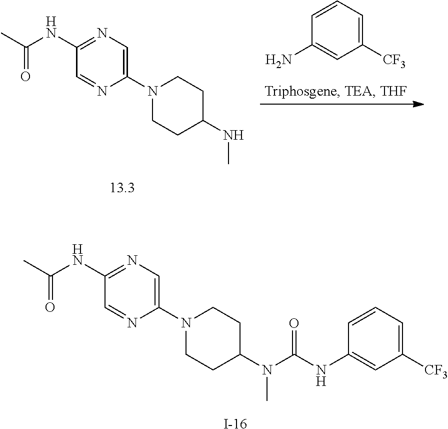

Synthesis of compound I-16. Compound I-16 was prepared from compound 13.3 and 3-(trifluoromethyl)aniline, following the procedure described in the synthesis of 1-9. The product was purified by flash column chromatography on silica gel (CombiFlash®, 2% methanol in DCM). MS(ES): m/z 437.31 [M+H]⁺; ¹H NMR (DMSO-d₆, 400 MHz): 10.32 (s, 1H), 8.79 (s, 1H), 8.63 (s, 1H), 8.14 (s, 1H), 7.97 (s, 1H), 7.79 (d, J=8 Hz, 1H), 7.48 (t, J=8 Hz, 1H), 7.29 (d, J=8 Hz, 1H), 4.42-4.33 (m, 3H), 2.92 (m, 2H), 2.83 (s, 3H), 2.08 (s, 3H), 1.72-1.68 (m, 4H).

### Example 17: 1-(1-(1H-pyrazolo[3,4-b]pyridin-5-yl)piperidin-4-yl)-1-methyl-3-(3-methyl-5-(trifluoromethyl)phenyl)urea

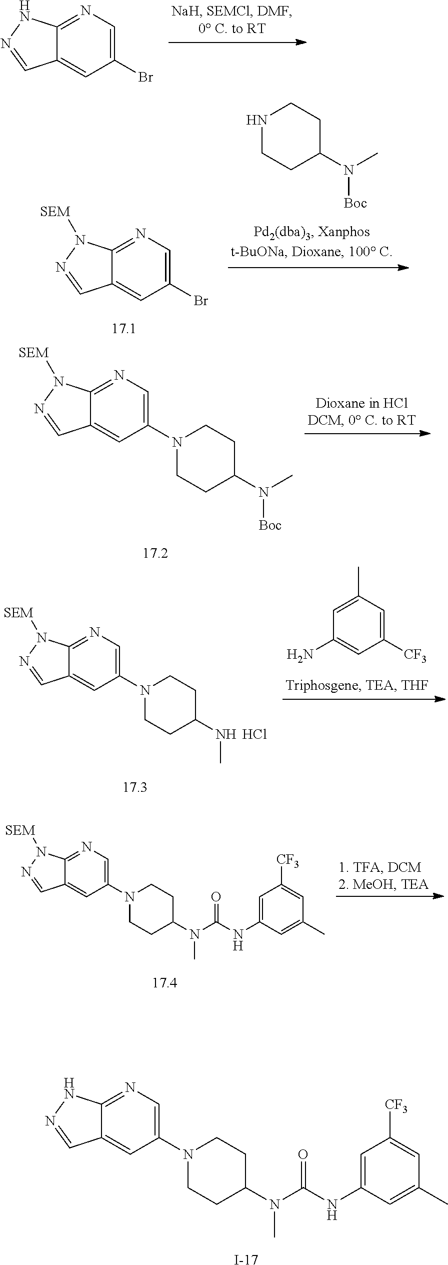

Synthesis of compound 17.1. To solution of 5-bromo-1H-pyrazolo[3,4-b]pyridine (2.0 g, 10.10 mmol, 1.0 equiv) in DMF (20 mL) was added sodium hydride (0.727 g, 15.15 mmol, 1.5 equiv) in small portions at 0° C. and stirred for 30 min. To the mixture was added 2-(trimethylsilyl)ethoxymethyl chloride (1.8 g, 11.11 mmol, 1.1 equiv), and the reaction mixture was stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (CombiFlash®, 10% ethyl acetate in hexane) to afford 17.1 (2.0 g, 60%), MS(ES): m/z 329.29 [M+H]⁺.

Synthesis of compound 17.2. A mixture of 17.1 (0.500 g, 1.5 mmol, 1.0 equiv) and tert-butyl methyl(piperidin-4-yl)carbamate (0.326 g, 1.5 mmol, 1.0 equiv) in 1,4-dioxane (6 mL) was degassed by bubbling through a stream of argon for 10 min. 4,5-Bis(diphenylphosphino)-9,9-dimethylxanthene (0.176 g, 0.304 mmol, 0.2 equiv), sodium tert-butoxide (0.438 g, 4.57 mmol, 3.0 equiv) and tris(dibenzylideneacetone)dipalladium(O) (0.139 g, 0.152 mmol, 0.1 equiv) were added and degassed for another 5 min. The reaction mixture was stirred at 100° C. for 2 h. It was cooled to room temperature, transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 35% ethyl acetate in hexane) to afford 17.2 (0.460 g, 65%). MS(ES): m/z 462.68 [M+H]⁺.

Synthesis of compound 17.3. To a solution of 17.2 (0.460 g, 0.997 mmol, 1.0 equiv) in DCM (5 mL) was added hydrochloric acid (4.0 M in dioxane, 4.6 mL) at 0° C. The reaction mixture was stirred at room temperature for 3 h. It was concentrated under reduced pressure to afford 17.3 (0.253 g, 70%). It was used in the next step without purification. MS(ES): m/z 362.57 [M+H]⁺.

Synthesis of compound 17.4. Compound 17.4 was prepared from compound 17.3 and 3-methyl-5-(trifluoromethyl)aniline, following the procedure described in the synthesis of I-9. The product was purified by flash column chromatography on silica gel (CombiFlash®, 2.2% methanol in DCM). MS(ES): m/z 563.71 [M+H]⁺.

Synthesis of 1-17. Compound 1-17 was prepared from compound 17.4, following the procedure described in the synthesis of I-11. The product was purified by flash column chromatography on silica gel (CombiFlash®, 3.2% methanol in DCM). MS(ES): m/z 433.38 [M+H]⁺, ¹H NMR (DMSO-d₆, 400 MHz): δ 13.41 (s, 1H), 8.68 (s, 1H), 8.43 (s, 1H), 7.99 (s, 1H), 7.78 (s, 1H), 7.66-7.62 (m, 2H), 7.11 (s, 1H), 4.22 (m, 1H), 3.70 (m, 2H), 2.89 (s, 3H), 2.80 (m, 2H), 2.36 (s, 3H), 1.94 (m, 2H), 1.71 (m, 2H).

### Example 18: N-(5-(4-(1-methyl-3-(1-methyl-2-oxo-5-(trifluoromethyl)-1,2-dihydropyridin-3-yl)ureido)piperidin-1-yl)pyrazin-2-yl)cyclopropanecarboxamide

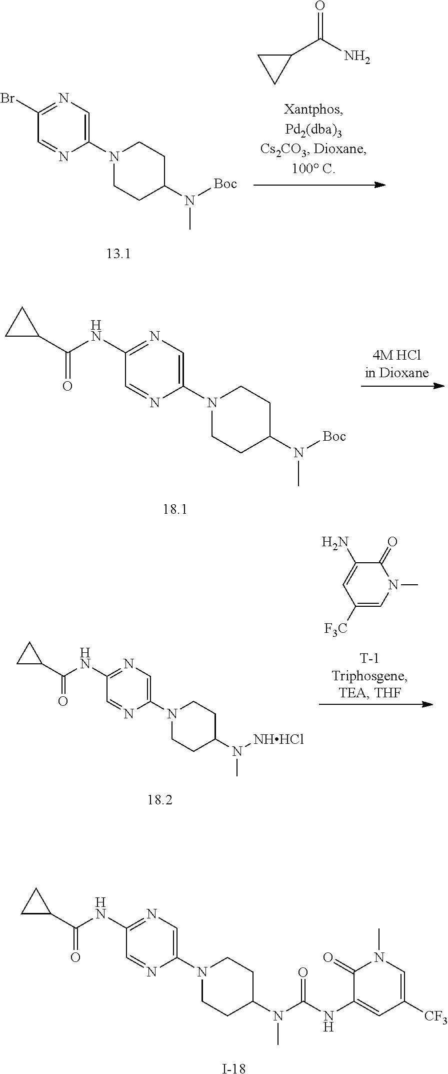

Synthesis of compound 18.1. A mixture of 13.1 (4.5 g, 12.12 mmol, 1 equiv), cyclopropylcarboxamide (3.09 g, 36.36 mmol, 3.0 equiv) and cesium carbonate (118 g, 36.8 mmol, 3 equiv) in dioxane (20 mL) was degassed by bubbling through a stream of argon for 10 min. 4,5-Bis(diphenylphosphino)-9,9-dimethylxanthene (1.40 g, 2.4 mmol, 0.2 equiv) and tris(dibenzylideneacetone)dipalladium(O) (1.10 g, 1.21 mmol, 0.1 equiv) were added and degassed for another 5 min. The reaction mixture was stirred at 100° C. for 90 min. It was cooled to room temperature and poured into water, stirred and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford 18.1 (3.1 g, 68%). MS (ES): m/z 376.34 [M+H]⁺.

Synthesis of compound 18.2. Compound 18.1 (1.5 g, 3.9 mmol, 1 equiv) was stirred with 4 M hydrogen chloride in dioxane (15 mL) at room temperature for 1.5 h. The reaction mixture was concentrated under reduced pressure to afford 18.2 (1.0 g, 80%). It was used in the next step without purification. MS(ES): m/z 312.14[M+H]⁺.

Synthesis of compound I-18. Compound I-18 was prepared from compound 18.2 and T-1, following the procedure described in the synthesis of 1-9. The product was purified by flash column chromatography on silica gel (CombiFlash®, 2% methanol in DCM). MS(ES): m/z 494.28 [M+H]⁺; ¹H NMR (DMSO-d₆, 400 MHz): 10.61 (s, 1H), 8.78 (s, 1H), 8.17 (s, 1H), 8.13 (s, 1H), 8.04 (s, 1H), 7.90 (s, 1H), 4.45-4.20 (m, 3H), 3.59 (s, 3H), 2.91-2.80 (m, 5H), 1.93 (m, 1H), 1.80-1.60 (m, 4H), 0.80-0.78 (m, 4H).

### Example 19: N-(5-(4-(1-methyl-3-(3-((4-methylpiperazin-1-yl)methyl)-5-(trifluoromethyl)phenyl)ureido)piperidin-1-yl)pyrazin-2-yl)acetamide

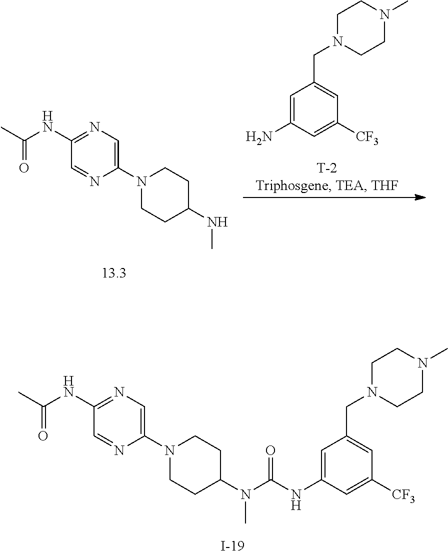

Synthesis of compound I-19. Compound I-19 was prepared from compound 13.3 and T-2, following the procedure described in the synthesis of 1-9. The product was purified by flash column chromatography on silica gel (CombiFlash®, 2% methanol in DCM). MS(ES): m/z 549.61 [M+H]⁺; ¹H NMR (DMSO-d₆, 400 MHz): 10.33 (s, 1H), 8.78 (s, 1H), 8.63 (s, 1H), 8.14 (s, 1H), 7.89 (s, 1H), 7.73 (s, 1H), 7.19 (s, 1H), 4.45-4.25 (m, 3H), 3.50 (s, 3H), 2.92 (m, 2H), 2.82 (s, 3H), 2.35 (m, 6H), 2.24 (m, 4H), 2.07 (s, 3H), 1.74-1.67 (m, 4H).

### Example 20: N-(5-(4-(1-methyl-3-(1-methyl-2-oxo-5-(trifluoromethyl)-1,2-dihydropyridin-3-yl)ureido)piperidin-1-yl)pyridin-2-yl)acetamide

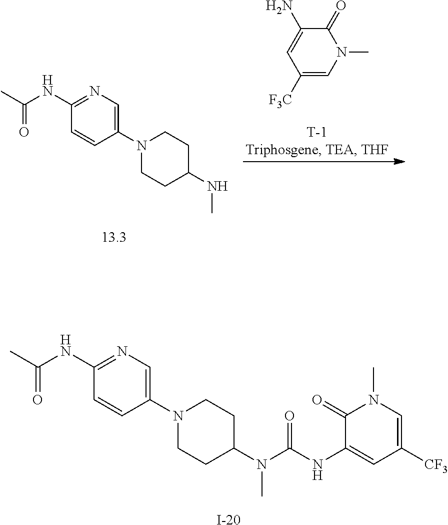

Synthesis of compound I-20. Compound I-20 was prepared from compound 12.4 and T-1, following the procedure described in the synthesis of 1-9. The product was purified by flash column chromatography on silica gel (CombiFlash®, 3.0% methanol in DCM). MS(ES): m/z: 467.24 [M+H]⁺; ¹H NMR (DMSO-d₆, 400 MHz): 10.24 (s, 1H), 8.17 (s, 1H), 8.02 (d, J=4 Hz, 2H), 7.91 (d, J=12 Hz, 2H), 7.42 (d, J=8 Hz, 1H), 4.18 (m, 1H), 3.76 (m, 2H), 3.59 (s, 3H), 2.87 (s, 3H), 2.71-2.67 (m, 2H), 2.04 (s, 3H), 1.82 (m, 2H), 1.65 (m, 2H).

### Example 21: 1-(1-(1H-pyrrolo[2,3-b]pyridin-5-yl)piperidin-4-yl)-1-methyl-3-(1-methyl-2-oxo-5-(trifluoromethyl)-1,2-dihydropyridin-3-yl)urea

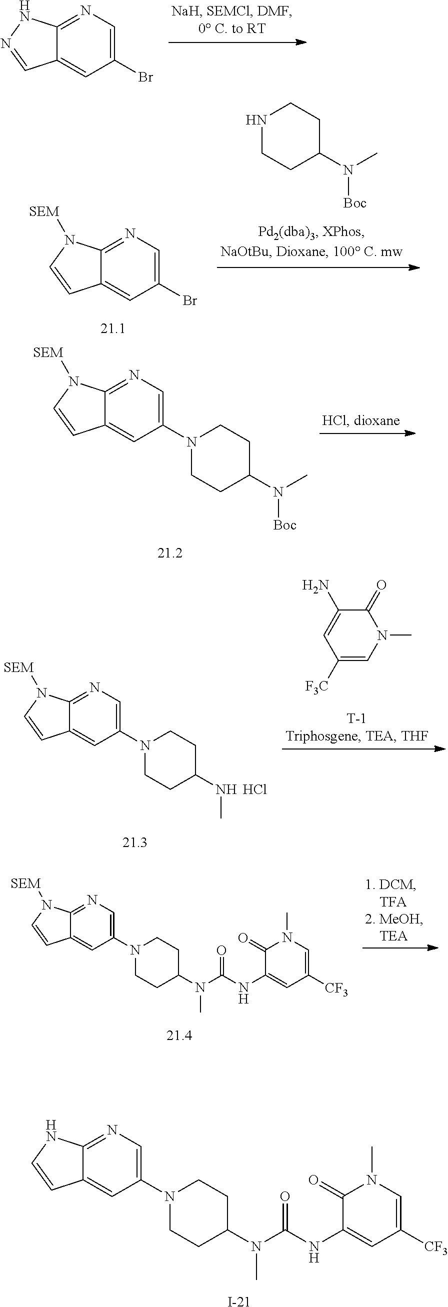

Synthesis of compound 21.1. To a solution of 5-bromo-1H-pyrrolo[2,3-b]pyridine (1 g, 5.076 mmol, 1.0 equiv) in DMF (15 mL) was added sodium hydride (60% in mineral oil, 0.243 g, 6.09 mmol, 1.2 equiv) at 0° C. and stirred for 15 min. To the mixture was added 2-(trimethylsilyl)ethoxymethyl chloride (1.05 g, 6.09 mmol, 1.2 equiv) and stirred at room temperature for 1 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford 21.1 (1.2 g, 72%). MS(ES): m/z 326 [M+1]⁺.

Synthesis of compound 21.2. Compound 21.2 was prepared from compound 21.1 and tert-butyl methyl(piperidin-4-yl)carbamate, following the procedure described in the synthesis of 11.2. The product was purified by flash column chromatography on silica gel (CombiFlash®, 20% ethyl acetate in hexane). MS(ES): m/z 460 [M+H]⁺.

Synthesis of compound 21.3. Compound 21.2 (0.3 g, 1.87 mmol, 1.0 equiv) was stirred with 4 N HCl in dioxane (5 mL) at room temperature for 1 h. The reaction mixture was concentrated under reduced pressure to afford 21.3 (0.280 g). It was used in the next step without purification. MS(ES): m/z 360 [M+H]⁺.

Synthesis of compound 21.4. Compound 21.4 was prepared from compound 21.3 and T-1, following the procedure described in the synthesis of I-9. The product was purified by flash column chromatography on silica gel (CombiFlash®, 5% methanol in DCM as). MS(ES): m/z 578 [M+1]⁺.

Synthesis of 1-21. Compound 1-21 was prepared from compound 21.4 following the procedure described in the synthesis of I-11. The product was purified by flash column chromatography on silica gel (CombiFlash®, 5% methanol in DCM). MS(ES): m/z 449.39 [M+H]⁺; ¹H NMR (DMSO-d₆, 400 MHz): δ 11.39 (s, 1H), 8.19 (s, 1H), 8.09 (s, 1H), 8.06 (s, H), 7.93 (s, 1H), 7.54 (s, 1H), 7.38 (s, 1H), 6.33 (s, 1H), 4.16 (m, 2H), 3.61 (s, 3H), 3.18 (m, 1H), 2.91 (s, 3H), 2.80-2.75 (m, 2H), 1.94 (m, 2H), 1.70 (m, 2H).

### Example 22: (R)-1-(3,3-difluoro-1-(pyrazin-2-yl)piperidin-4-yl)-1-methyl-3-(1-methyl-2-oxo-5-(trifluoromethyl)-1,2-dihydropyridin-3-yl)urea and (S)-1-(3,3-difluoro-1-(pyrazin-2-yl)piperidin-4-yl)-1-methyl-3-(1-methyl-2-oxo-5-(trifluoromethyl)-1,2-dihydropyridin-3-yl)urea

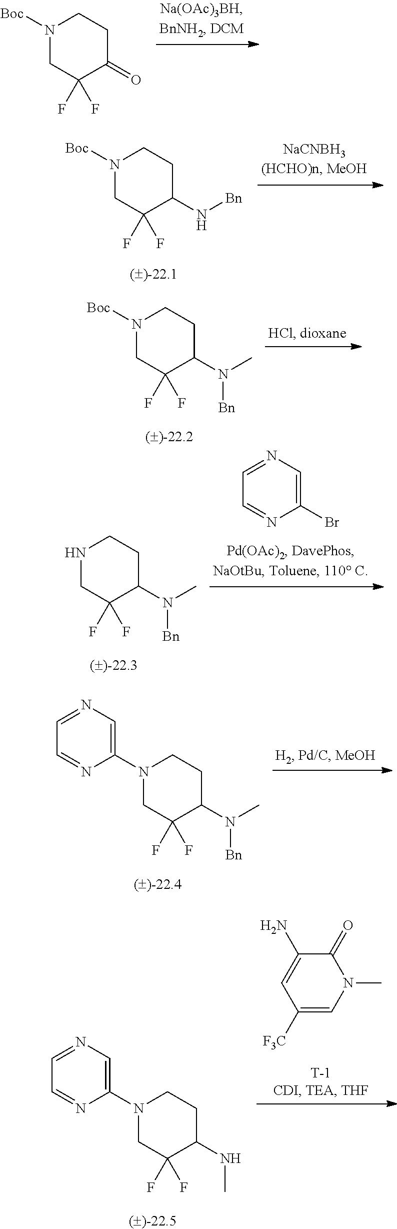

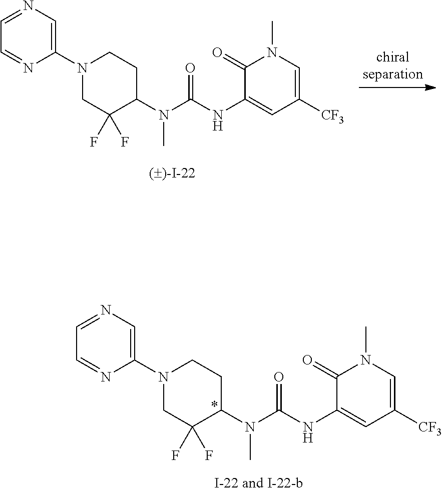 [SMILES: CN(C(=O)Nc1cc(C(F)(F)F)cn(C)c1=O)C1CCN(c2cnccn2)CC1(F)F ; CN(C(=O)Nc1cc(C(F)(F)F)cn(C)c1=O)C1CCN(c2cnccn2)CC1(F)F]

Synthesis of compound (±)-22.1. To a solution of tert-butyl 3,3-difluoro-4-oxopiperidine-1-carboxylate (5 g, 21.25 mmol, 1.0 equiv) in DCM (100 mL) was added benzyl amine (3.41 g, 31.88 mmol, 1.5 equiv) and sodium triacetoxyborohydride (18 g, 85.02 mmol, 4.0 equiv). The reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford (±)-22.1 (3.9 g, 56%). MS(ES): m/z 327 [M+1]⁺.

Synthesis of compound (±)-22.2. To a solution of (±)-22.1 (3.8 g, 11.62 mmol, 1.0 equiv) and paraformaldehyde (3.48 g, 116.2 mmol, 10 equiv) in methanol (75 mL) was added sodium cyanoborohydride (3.65 g, 58.10 mmol, 5.0 equiv). The reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 28% ethyl acetate in hexane) to afford (±)-22.2 (3.1 g, 78%). MS(ES): m/z 341 [M+H]⁺.

Synthesis of compound (±)-22.3. To a solution of (±)-22.2 (3.1 g, 2.71 mmol, 1.0 equiv) in methanol (31 mL) was added hydrochloric acid (4.0 M in dioxane, 30 mL). The reaction mixture was stirred at room temperature for 3 h. It was transferred into saturated sodium bicarbonate solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 4% methanol in DCM) to afford (±)-22.3 (1.6 g, 73%). MS(ES): m/z 241 [M+H]⁺.

Synthesis of compound (±)-22.4. A mixture of (±)-22.3 (1.0 g, 4.16 mmol, 1.3 equiv), 2-bromopyrazine (0500 g, 3.14 mmol, 1.0 equiv), and sodium tert-butoxide (0.36 g, 3.77 mmol, 1.2 equiv) in toluene (20 mL) was degassed by bubbling through a stream of argon for 15 min. DavePhos (0.037 g, 0.094 mmol, 0.03 equiv) and palladium(II) acetate (0.021 g, 0.094 mmol, 0.03 equiv) and degassed for another 10 min. The reaction mixture was stirred at 110° C. for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford (±)-22.4 (0.750 g, 57%). MS(ES): m/z 319.2 [M+H]⁺.

Synthesis of compound (±)-22.5. A mixture of (±)-22.4 (0.750 g, 2.35 mmol, 1.0 equiv) and (10% palladium on charcoal, 0.375 g) in methanol (10 mL) was stirred under hydrogen (1 atm) at room temperature for 16 h. The reaction mixture was filtered through a pad of Celite®. The filtrate was concentrated under reduced pressure to afford (±)-22.5 (0.350 g, 65%). MS(ES): m/z 229.12 [M+H]⁺.

Synthesis of compound I-22-a and I-22-b. To a solution of (±)-22.5 (0.300 g, 1.56 mmol, 1.0 equiv), T-1 (0.356 g, 1.56 mmol, 1.0 equiv), and trimethylamine (2.36 g, 23.4 mmol, 15.0 equiv) in THE (10 mL) was added 1,1′-carbonyldiimidazole (1.77 g, 1.56 mmol, 1.0 equiv) at room temperature. The reaction mixture stirred for 4 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford (±)-I-22 (0.150 g, 26%). MS(ES): m/z 447.21 [M+H]⁺.

The racemate was separated by chiral HPLC (column: CHIRALPAK IBN-5 (250 mm*4.6 mm, 5 μm); mobile phase: (A) 0.1% diethylamine in n-hexane, (B) 0.1% diethylamine in isopropanol: methanol (1:1); flow rate: 20 mL/min) to afford first eluting fraction (1-22-a) and second eluting fraction (I-22-b). (*Absolute configuration was not determined.)

I-22-a: MS(ES): m/z: 447.20 [M+H]⁺; ¹H NMR (DMSO-d₆, 400 MHz): δ 8.50 (s, 1H), 8.17 (d, J=2.4 Hz, 1H), 8.13 (s, 1H), 8.09 (s, 1H), 8.06 (s, 1H), 7.91 (d, J=2.4 Hz, 1H), 4.95 (m, 1H), 4.82 (m, 1H), 4.55 (m, 1H), 3.61 (s, 3H), 3.25 (m, 2H), 2.98 (s, 3H), 2.11 (m, 1H), 1.88 (m, 1H).

I-22-b: MS(ES): m/z: 447.19 [M+H]⁺, ¹H NMR (DMSO-d₆, 400 MHz): δ 8.50 (s, 1H), 8.17 (d, J=2.4 Hz, 1H), 8.13 (s, 1H), 8.09 (s, 1H), 8.06 (s, 1H), 7.91 (d, J=2.4 Hz, 1H), 4.95 (m, 1H), 4.82 (m, 1H), 4.55 (m, 1H), 3.61 (s, 3H), 3.25 (m, 2H), 2.98 (s, 3H), 2.11 (m, 1H), 1.88 (m, 1H).

### Example 23: N-(5-(4-(1-methyl-3-(6-methyl-4-(trifluoromethyl)pyridin-2-yl)ureido)piperidin-1-yl)pyrazin-2-yl)acetamide

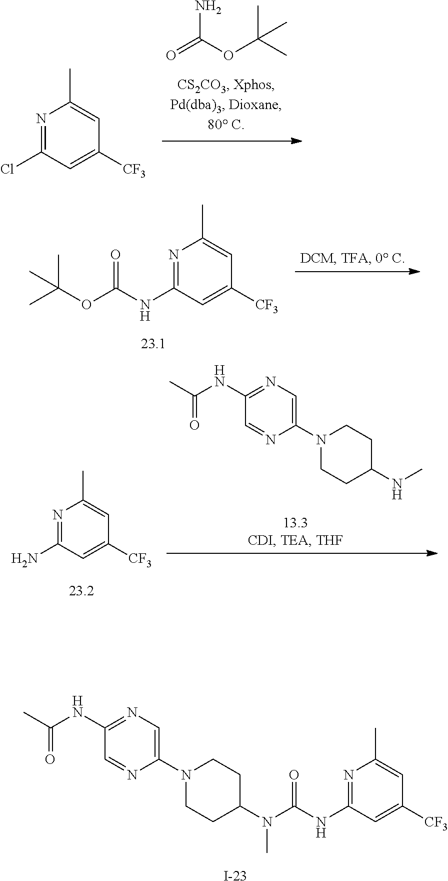

Synthesis of compound 23.1. A mixture of 2-chloro-6-methyl-4-(trifluoromethyl)pyridine (1.0 g, 5.12 mmol, 1.0 equiv), tert-butyl carbamate (1.2 g, 10.25 mmol, 2.0 equiv) and cesium carbonate (5.8 g, 17.94 mmol, 3.5 equiv) in 1,4-dioxane (10 mL) was degassed by bubbling through a stream of argon for 10 min. XPhos (0.245 g, 0.512 mmol, 0.1 equiv) and tris(dibenzylideneacetone)dipalladium(O) (0.234 g, 0.256 mmol, 0.05 equiv) were added and degassed for another 5 min. The reaction mixture stirred at 80° C. for 2 h. It was cooled to room temperature, transferred into water, and extracted with ethyl acetate. The combined organic layers were washed with brine, dried over anhydrous sodium sulfate, filtered, and concentrated under reduced pressure to afford. The residue was purified by flash column chromatography on silica gel (CombiFlash®, 3.8% methanol in DCM) to afford 23.1 (0.600 g, 42.5%). MS(ES): m/z: 276.26 [M+H]⁺.

Synthesis of compound 23.2. To solution of 23.1 (0.600 g, 2.895 mmol, 1.0 equiv) in DCM (6 mL) was added trifluoroacetic acid (6.0 mL) at 0° C. and stirred for 1 h. It was transferred into ice-cold saturated sodium bicarbonate solution and extracted with DCM. The combined organic layers were washed with brine, dried over anhydrous sodium sulfate, filtered, and concentrated under reduced pressure to afford 23.2 (0.250 g, 65%). MS(ES): m/z: 177.2 [M+H]⁺.

Synthesis of compound I-23. Compound I-23 was prepared from compound 23.2 and 13.3, following the procedure described in the synthesis of I-12. The product was purified by flash column chromatography on silica gel (CombiFlash®, 3.0% methanol in DCM). MS(ES): m/z: 452.4 [M+H]⁺; ¹H NMR (DMSO-d₆, 400 MHz): δ 10.32 (s, 1H), 9.38 (s, 1H), 8.77 (s, 1H), 8.12 (s, 1H), 7.36 (s, 1H), 6.83 (s, 1H), 4.36 (m, 3H), 3.35 (s, 3H), 2.95-2.70 (m, 5H), 2.06 (s, 3H), 1.55 (m, 4H).

### Example 24: N-(5-(4-(1-methyl-3-(1-methyl-2-oxo-5-(trifluoromethyl)-1,2-dihydropyridin-3-yl)ureido)piperidin-1-yl)pyridin-2-yl)cyclopropanecarboxamide

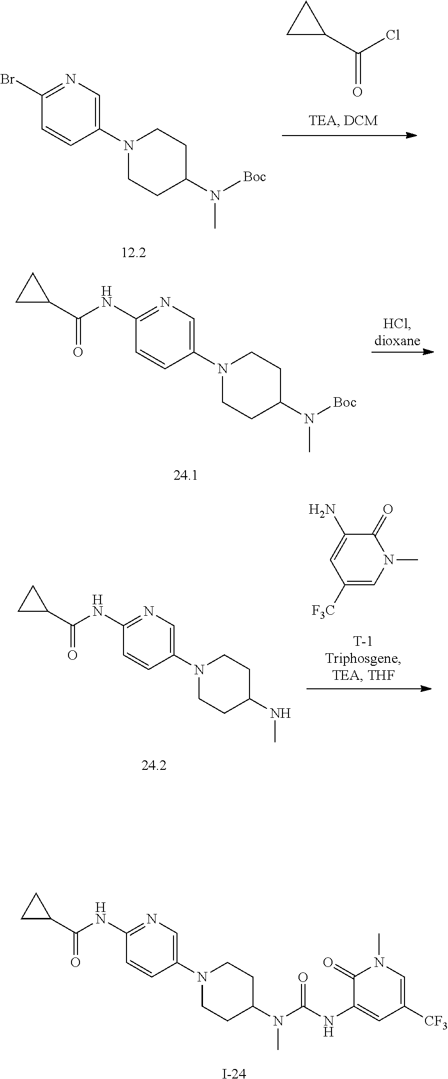

Synthesis of compound 24.1. To a solution of 12.2 (0.7 g, 2.28 mmol, 1.0 equiv) in DCM (9 mL) was added cyclopropanecarbonyl chloride (0.262 g, 2.51 mmol, 1.1 equiv), followed by triethylamine (0.310 g, 2.721 mmol, 3.0 equiv) at 0° C. The reaction mixture was stirred at room temperature for 30 min. It was transferred into ice-water and extracted with DCM. The combined organic layers were washed with brine, dried over anhydrous sodium sulfate, filtered, and concentrated under reduced pressure. The residue was purified by flash column chromatography on silica gel (CombiFlash®, 35% ethyl acetate in hexane) to afford 24.1 (0.4 g, 47%). MS(ES) m/z 375.67 [M+H]⁺.

Synthesis of compound 24.2. Compound 24.1 (0.4 g, 1.07 mmol, 1.0 equiv) was stirred with hydrogen chloride (4 M in 1,4-dioxane, 10 mL) at room temperature for 8 h. It was transferred and concentrated under reduced pressure to afford 24.2 (0.20 g, 68%). It was used without purification. MS(ES): m/z 275.36 [M+H]⁺.

Synthesis of I-24. Compound I-24 was prepared from compound 24.2 and T-1, following the procedure described in the synthesis of 1-9. The product was purified by flash column chromatography on silica gel (CombiFlash®, 4.2% methanol in DCM). MS(ES): m/z: 493.13 [M+H]⁺; ¹H NMR (DMSO-d₆, 400 MHz): δ 10.55 (s, 1H), 8.18 (d, J=4 Hz, 1H), 8.04 (d, J=4 Hz, 2H), 7.93 (d, J=8 Hz, 2H), 7.44 (t, 1H), 4.20-4.12 (m, 3H), 3.79-3.76 (m, 2H), 3.60 (s, 3H), 2.89 (s, 3H), 2.76-2.73 (m, 1H), 1.95-1.86 (m, 2H), 1.68-1.65 (m, 2H), 1.25 (m, 2H), 0.77 (m, 2H).

### Example 25: 1-(1-(6-cyclopropyl-5H-pyrrolo[2,3-b]pyrazin-2-yl)piperidin-4-yl)-1-methyl-3-(1-methyl-2-oxo-5-(trifluoromethyl)-1,2-dihydropyridin-3-yl)urea

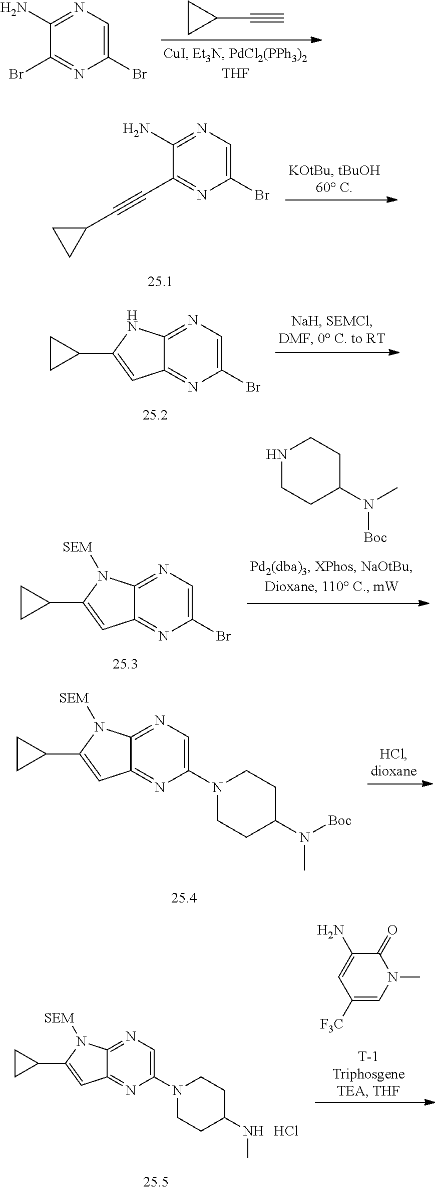

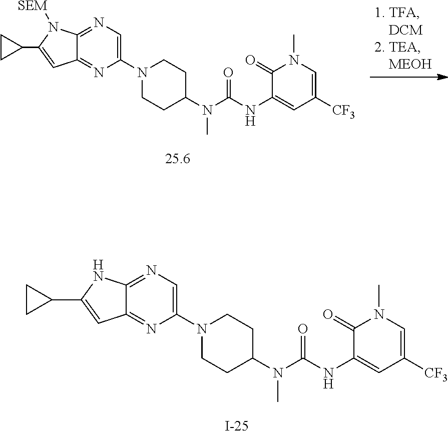

Synthesis of compound 25.1. A mixture of 3,5-dibromopyrazin-2-amine (3.0 g, 11.86 mmol, 1.0 equiv), triethylamine (4.95 mL, 35.35 mmol, 3.0 equiv), and copper iodide (0.225 g, 1.185 mmol, 0.1 equiv) in THE (60 mL) was degassed by bubbling through a stream of argon for 5 min. Palladium(II)bis(triphenylphosphine) dichloride (0.866 g, 1.185 mmol, 0.1 equiv) and ethynylcyclopropane (0.822 g, 12.45 mmol, 1.05 equiv) were added and degassed with argon for another 5 min. The reaction mixture was stirred at room temperature for 3 h. The reaction mixture was poured in water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 8% ethyl acetate in hexane) to afford 25.1 (1.2 g, 42.5%). MS(ES): m/z 238.09 [M+H]⁺.

Synthesis of compound 25.2. To a solution of 25.1 (1.2 g, 5.04 mmol, 1.0 equiv) in tert-butanol (15 mL) was added potassium tert-butoxide (1.24 g, 11.09 mmol, 2.2 equiv). The reaction mixture was stirred at 60° C. for 2 h. It was poured into aqueous 5 N hydrogen chloride solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford 25.2 (0.900 g, 75%). MS(ES): m/z 238.05 [M+H]⁺.

Synthesis of compound 25.3. To a solution of 25.2 (0.900 g, 3.78 mmol, 1.0 equiv) in DMF (20 mL) was added sodium hydride (0.227 g, 5.67 mmol, 1.5 equiv) at 0° C. and stirred for 30 min. To the mixture was added 2-(trimethylsilyl)ethoxymethyl chloride (1.26 g, 7.56 mmol, 2.0 equiv) and stirred at room temperature for 2 h. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 7.7% ethyl acetate in hexane) to afford 25.3 (0.950 g, 68%). MS(ES): m/z 368.35 [M+H]⁺.

Synthesis of compound 25.4. A mixture of 25.3 (0.950 g, 2.57 mmol, 1.0 equiv), tert-butyl methyl(piperidin-4-yl)carbamate (0.607 g, 2.83 mmol, 1.1 equiv), and sodium tert-butoxide (0.620 g, 6.44 mmol 2.5 equiv) in 1,4-dioxane (5 mL) was degassed by bubbling through a stream of argon for 10 min. 2-Dicyclohexylphosphino-2′,4′,6′-triisopropylbiphenyl (0.245 g, 0.515 mmol, 0.2 equiv) and tris(diphenylideneacetone)dipalladium(O) (0.236 g, 0.257 mmol, 0.1 equiv) were added and degassed with argon for another 10 min. The reaction mixture was stirred at 110° C. in a microwave reactor for 1 h. The reaction mixture was poured in water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford 25.4 (0.450 g, 35%). MS(ES): m/z 501.75 [M+H]⁺.

Synthesis of compound 25.5. Compound 25.4 (0.450 g, 0.896 mmol, 1.0 equiv) was stirred with 4 M hydrochloric acid in dioxane (10 mL) at room temperature for 1.5 h. The reaction mixture was concentrated under reduced pressure to afford 25.5 (0.250 g, 69%). It was used in the next step without purification. MS (ES): m/z 401.75 [M+H]⁺.

Synthesis of compound 25.6. Compound 25.6 was prepared from compound 25.5 and T-1, following the procedure described in the synthesis of I-9. The product was purified by flash column chromatography on silica gel (CombiFlash®, 2% methanol in DCM). MS (ES): m/z 619.77 [M+H]⁺.

Synthesis of compound I-25. Compound I-25 was prepared from compound 25.6 following the procedure described in the synthesis of I-11. The product was purified by flash column chromatography on silica gel (CombiFlash®, 5.6% methanol in DCM). MS(ES): m/z 490.48 [M+H]⁺; ¹H NMR (DMSO-d₆, 400 MHz): δ 11.67 (s, 1H), 8.17 (s, 1H), 8.03 (s, 1H), 7.89 (s, 1H), 7.86 (s, 1H), 6.00 (s, 1H), 4.40-4.20 (m, 3H), 3.59 (s, 3H), 2.83 (s, 3H), 2.00 (m, 1H), 1.74 (m, 2H), 1.65 (m, 2H), 1.232 (m, 2H), 0.86 (m, 2H), 0.85 (m, 2H).

### Example 26: 1-(1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

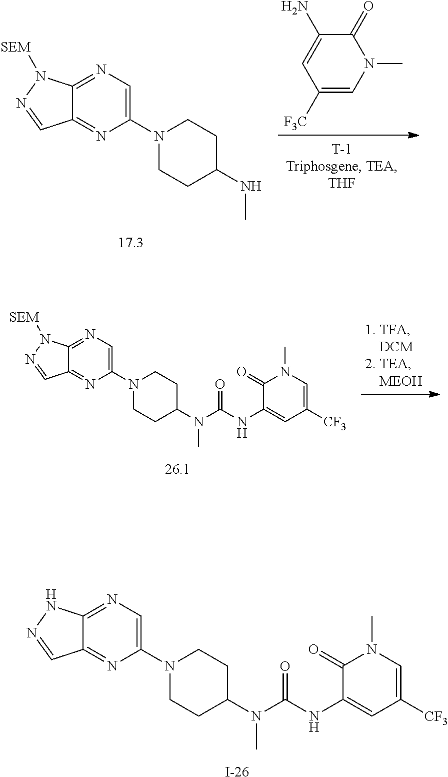

Synthesis of compound 26.1. Compound 26.1 was prepared from compound 17.3 and T-1 following the procedure described in the synthesis of I-9. The product was purified by silica gel flash column chromatography on silica gel (CombiFlash®, 3.7% methanol in DCM). MS(ES): m/z 580.70 [M+H]⁺.

Synthesis of I-26. Compound I-26 was prepared from compound 26.1 following the procedure described in the synthesis of I-11. The product was purified by flash column chromatography on silica gel (CombiFlash®, 2.8% methanol in DCM). MS(ES): m/z: 450.4 [M+H]⁺; ¹H NMR (DMSO-d₆, 400 MHz): δ 13.39 (s, 1H), 8.46 (s, 1H), 8.18 (s, 1H), 8.04 s, 1H), 7.97 (s, 1H), 7.92 (s, 1H), 7.64 (s, 1H), 4.19 (m, 1H), 3.68 (m, 2H), 3.60 (s, 3H), 2.91 (s, 3H), 2.78 (m, 2H), 1.92 (m, 2H), 1.70 (m, 2H).

### Example 27: (R)-1-(3,3-difluoro-1-(5H-pyrrolo[2,3-b]pyrazin-2-yl)piperidin-4-yl)-1-methyl-3-(1-methyl-2-oxo-5-(trifluoromethyl)-1,2-dihydropyridin-3-yl)urea and (S)-1-(3,3-difluoro-1-(5H-pyrrolo[2,3-b]pyrazin-2-yl)piperidin-4-yl)-1-methyl-3-(1-methyl-2-oxo-5-(trifluoromethyl)-1,2-dihydropyridin-3-yl)urea

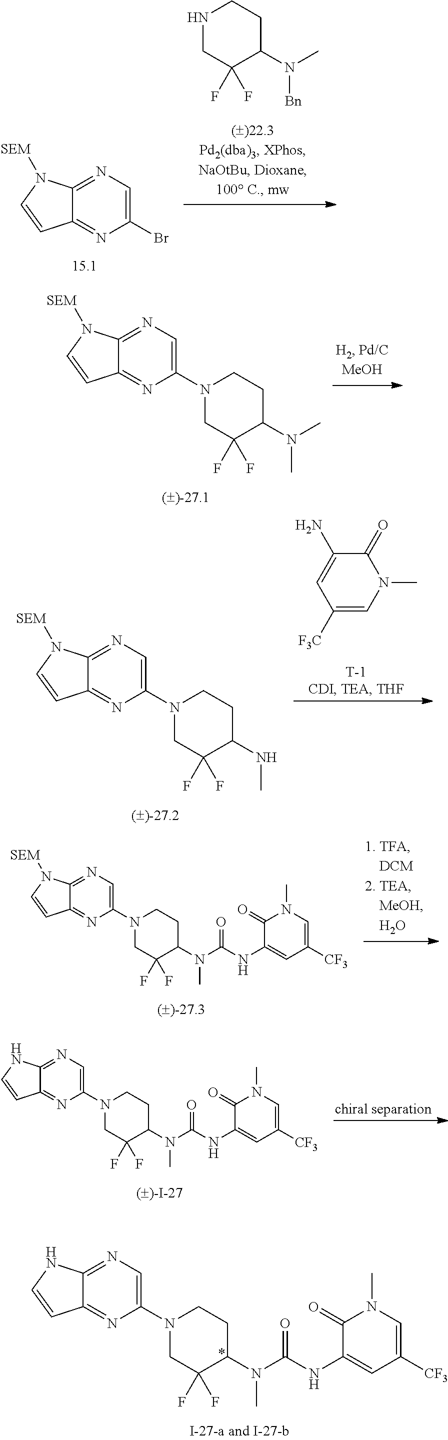

Synthesis of compound (±)-27.1. Compound (±)-27.1 was prepared from compound 15.1 and (±)-22.3, following the procedure described in the synthesis of 11.2. The product was purified by flash column chromatography on silica gel (CombiFlash®, 20% ethyl acetate in hexane). MS(ES): m/z 488.6 [M+H]⁺.

Synthesis of compound (±)-27.2. A mixture of (±)-27.1 (1.0 g, 2.05 mmol, 1.0 equiv) and 10% palladium on charcoal (0.500 g) in methanol (10 mL) was stirred under hydrogen (1 atm) for 16 h. The reaction mixture was filtered through a pad of Celite®. The filtrate was concentrated under reduced pressure to afford (±)-27.2 (0.700 g, 86%). MS(ES): m/z 398.45 [M+H]⁺.

Synthesis of compound (±)-27.3. Compound (±)-27.3 was prepared from compound (±)-27.2 and T-1, following the procedure described in the synthesis of I-12. The product was purified by flash column chromatography on silica gel (CombiFlash®, 5% methanol in DCM). MS(ES): m/z 616.5 [M+H]⁺.

Synthesis of I-27-a and I-27-b. Compound (±)-27 was prepared from compound (±)-27.3 following the procedure described in the synthesis of I-11. The product was purified by flash column chromatography on silica gel (CombiFlash®, 5% methanol in DCM). The racemate was separated by chiral HPLC (column: CHIRALPAK IBN-5 (250 mm*4.6 mm, 5 μm); mobile phase: (A) 0.1% diethylamine in n-hexane, (B) 0.1% diethylamine in isopropanol: methanol (1:1); flow rate: 20 mL/min) to afford first eluting fraction (I-27-a) and second eluting fraction (I-27-b). (*Absolute configuration was not determined.)

I-27-a: MS(ES): m/z: 486.39 [M+H]⁺; ¹H NMR (DMSO-d₆, 400 MHz): δ 11.65 (s, 1H), 8.15 (s, 2H), 8.08 (s, 1H), 8.06 (s, 1H), 7.63 (s, 1H), 6.36 (s, 1H), 4.91-4.87 (m, 1H), 4.69-4.63 (m, 1H), 4.47-4.44 (m, 1H), 3.60 (s, 3H), 3.14 (m, 2H), 2.98 (s, 3H), 2.21-2.19 (m, 1H), 1.87 (m, 1H).

I-27-b: MS(ES): m/z: 486.38 [M+H]⁺; ¹H NMR (DMSO-d₆, 400 MHz): δ 11.65 (s, 1H), 8.15 (s, 2H), 8.08 (s, 1H), 8.06 (s, 1H), 7.63 (s, 1H), 6.36 (s, 1H), 4.91-4.87 (m, 1H), 4.69-4.63 (m, 1H), 4.47-4.44 (m, 1H), 3.60 (s, 3H), 3.14 (m, 2H), 2.98 (s, 3H), 2.21-2.19 (m, 1H), 1.87 (m, 1H).

The following compounds were prepared following the procedure of I-27:

| Cmpd. | | |
|---|---|---|
| No. | Structure and Name | Analytical Data |
| I-33-aª | 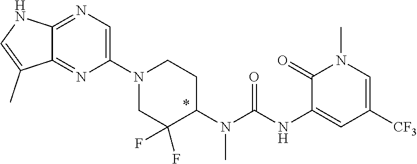 [SMILES: Cc1c[nH]c2ncc(N3CCC(N(C)C(=O)Nc4cc(C(F)(F)F)cn(C)c4=O)C(F)(F)C3)nc12] | MS (ES): m/z 500.40 [M + H]⁺ ¹H NMR (DMSO-d₆, 400 MHz): δ 11.30 (s, 1H), 8.16 (s 1H), 8.11 (s, 1H), 8.08 (s, 1H), 8.06 (s, 1H), 7.42 (s, 1H), 4.94-4.84 (m, 1H), 4.71-4.46 (t, J = 12.0 Hz, 1H), 4.49-4.46 (d, J = 12.4 Hz, 1H), 3.60 (s, 3H), 3.30-3.24 (m, 1H), 3.18-3.09 (m, 2H), 3.00 (s, 3H), 2.20 (s, 3H), 1.91-1.83 (m, 1H) |
| | (R)- or (S)-1-(3,3-difluoro-1-(7-methyl- | |
| | 5H-pyrrolo[2,3-b]pyrazin-2- | |
| | yl)piperidin-4-yl)-1-methyl-3-(1- | |
| | methyl-2-oxo-5-(trifluoromethyl)-1,2- | |
| | dihydropyridin-3-yl)urea | |
| I-33-b^{b} | 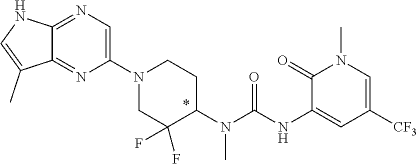 [SMILES: Cc1c[nH]c2ncc(N3CCC(N(C)C(=O)Nc4cc(C(F)(F)F)cn(C)c4=O)C(F)(F)C3)nc12] | MS (ES): m/z 500.41 [M + H]⁺ ¹H NMR (DMSO-d6, 400 MHz): δ 11.30 (s, 1H), 8.16 (s 1H), 8.11 (s, 1H), 8.08 (s, 1H), 8.06 (s, 1H), 7.42 (s, 1H), 4.90-4.86 (m, 1H), 4.71-4.65 (t, J = 12.0 Hz, 1H), 4.49-4.46 (d, J = 12.4 Hz, 1H), 3.60 (s, 3H), 3.27-3.23 (m, 1H), 3.17-3.09 (m, 2H), 3.00 (s, 3H), 2.20 (s, 3H), 1.91-1.82 (m, 1H) |
| | (S)- or (R)-1-(3,3-difluoro-1-(7-methyl- | |
| | 5H-pyrrolo[2,3-b]pyrazin-2- | |
| | yl)piperidin-4-yl)-1-methyl-3-(1- | |
| | methyl-2-oxo-5-(trifluoromethyl)-1,2- | |
| | dihydropyridin-3-yl)urea | |
| 1-36-aª | 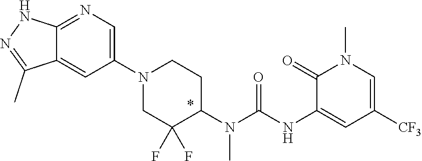 [SMILES: Cc1n[nH]c2ncc(N3CCC(N(C)C(=O)Nc4cc(C(F)(F)F)cn(C)c4=O)C(F)(F)C3)cc12] | MS (ES): m/z 500.33 [M + H]⁺ ¹H-NMR (400 MHz, DMSO-d6): δ 12.98 (s, 1H), 8.43 (s, 1H), 8.15 (s, 1H), 8.07 (s, 1H), 8.07 (s, 1H) 7.72 (s, 1H), 4.82 (s, 1H), 3.91 (s, 1H), 3.77 (d, J = 8.0 Hz, 1H), 3.60 (s, 3H), 3.22-3.20 (m, 2H), 3.10 (s, 3H), 2.46 (s, 3H), 2.31 (s, 1H), 1.88 (s, 1H) |
| | (R)- or (S)-1-(3,3-difluoro-1-(3-methyl- | |
| | 1H-pyrazolo[3,4-b]pyridin-5- | |
| | yl)piperidin-4-yl)-1-methyl-3-(1- | |
| | methyl-2-oxo-5-(trifluoromethyl)-1,2- | |
| | dihydropyridin-3-yl)urea | |
| I-36-b^{b} | 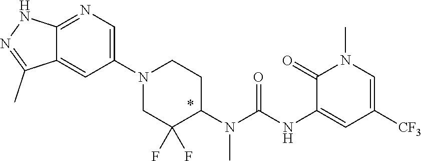 [SMILES: Cc1n[nH]c2ncc(N3CCC(N(C)C(=O)Nc4cc(C(F)(F)F)cn(C)c4=O)C(F)(F)C3)cc12] | MS (ES): m/z 500.33 [M + H]⁺ ¹H-NMR (400 MHz, DMSO-d6): δ 12.98 (s, 1H), 8.43 (s, 1H), 8.11 (s, 1H), 8.15 (s, 1H), 8.07 (s, 1H), 7.72 (s, 1H), 4.79 (s, 1H), 3.94- 3.91 (d, J = 12.00 Hz, 1H), 3.78-3.76 (d, J = 8.0 Hz, 1H), 3.59 (s, 3H), 3.27-3.17 (m, 2H), 3.05 (s, 3H), 2.49 (s, 3H), 2.30 (d, J = 12.0 Hz, 1H), 1.87 (s, 1H) |
| | (S)- or (R)-1-(3,3-difluoro-1-(3-methyl- | |
| | 1H-pyrazolo[3,4-b]pyridin-5- | |
| | yl)piperidin-4-yl)-1-methyl-3-(1- | |
| | methyl-2-oxo-5-(trifluoromethyl)-1,2- | |
| | dihydropyridin-3-yl)urea | |
| I-37-a^{e} | 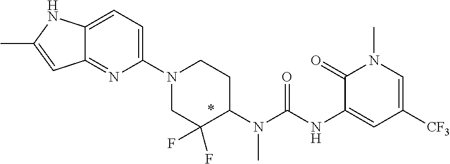 [SMILES: Cc1cc2nc(N3CCC(N(C)C(=O)Nc4cc(C(F)(F)F)cn(C)c4=O)C(F)(F)C3)ccc2[nH]1] | MS (ES): m/z 500.32 [M + H]⁺ ¹H-NMR (400 MHz, DMSO-d6): δ 11.47 (s, 1H), 8.18-8.15 (m, 1H), 8.07-7.96 (m, 3H), 6.11-6.07 (m, 1H), 4.92-4.81 (m, 1H), 4.68- 4.55 (m, 1H), 4.46-4.36 (s, 1H), 3.59 (s, 3H), 3.37-3.33 (m, 2H), 2.98 (s, 3H), 2.40 (s, 3H), 2.29-2.13 (m, 1H), 2.41-2.26 (m, 1H) |
| | (R)- or (S)-1-(3,3-difluoro-1-(6-methyl- | |
| | 5H-pyrrolo[2,3-b]pyrazin-2- | |
| | yl)piperidin-4-yl)-1-methyl-3-(1- | |
| | methyl-2-oxo-5-(trifluoromethyl)-1,2- | |
| | dihydropyridin-3-yl)urea | |
| I-37-b^{f} | 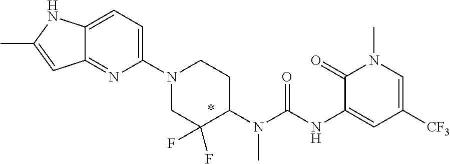 [SMILES: Cc1cc2nc(N3CCC(N(C)C(=O)Nc4cc(C(F)(F)F)cn(C)c4=O)C(F)(F)C3)ccc2[nH]1] | MS (ES): m/z 500.29 [M + H]⁺ ¹H-NMR (400 MHz, DMSO-d6): δ 11.46 (s, 1H), 8.15 (s, 1H), 8.07 (s, 1H), 8.05 (s, 1H), 7.97 (s, 1H), 6.08 (s, 1H), 4.94-4.81 (m, 1H), 4.65-4.58 (m, 1H), 4.42-4.38 (d, J = 12.4 Hz, 1H), 3.59 (s, 3H), 3.24-3.06 (m, 2H), 2.98 (s, 3H), 2.40 (s, 3H), 2.20-2.18 (m, 1H), 1.89-1.81 (m, 1H) |
| | (S)- or (R)-1-(3,3-difluoro-1-(6-methyl- | |
| | 5H-pyrrolo[2,3-b]pyrazin-2- | |
| | yl)piperidin-4-yl)-1-methyl-3-(1- | |
| | methyl-2-oxo-5-(trifluoromethyl)-1,2- | |
| | dihydropyridin-3-yl)urea | |
| I-41-aª | 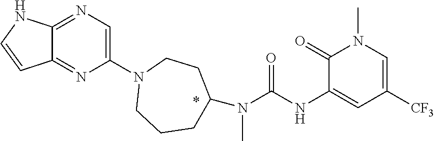 [SMILES: CN(C(=O)Nc1cc(C(F)(F)F)cn(C)c1=O)C1CCCN(c2cnc3[nH]ccc3n2)CC1] | MS (ES): m/z 464.37 [M + H]⁺ ¹H-NMR (400 MHz, DMSO-d6): δ 11.45 (s, 1H), 8.13 (s, 1H), 8.02 (s, 1H), 7.83-7.81 (d, J = 8.0 Hz, 2H), 7.53 (s, 1H), 6.29 (s, 1H), 4.11-4.19 (m, 1H), 3.90-3.83 (m, 1H), 3.58 (s, 3H), 2.84 (s, 3H), 1.97-1.86 (m, 2H), 1.78- 1.63 (m, 2H), 1.35-1.01 (m, 5H) |
| | (R)- or (S)-1-(1-(5H-pyrrolo[2,3- | |
| | b]pyrazin-2-yl)azepan-4-yl)-1-methyl- | |
| | 3-(1-methyl-2-oxo-5-(trifluoromethyl)- | |
| | 1,2-dihydropyridin-3-yl)urea | |
| I-41-b^{b} | 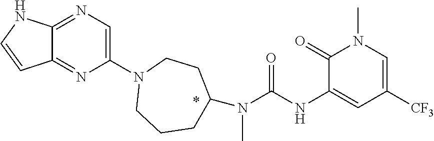 [SMILES: CN(C(=O)Nc1cc(C(F)(F)F)cn(C)c1=O)C1CCCN(c2cnc3[nH]ccc3n2)CC1] | MS (ES): m/z 464.36 [M + H]⁺ ¹H-NMR (400 MHz, DMSO-d6): δ 11.44 (s, 1H), 8.12 (s, 1H), 8.01 (s, 1H), 7.82-7.81 (d, J = 8.0 Hz, 2H), 7.53 (s, 1H), 6.29 (s, 1H), 4.12-4.19 (m, 1H), 3.86-3.82 (m, 1H), 3.58 (s, 3H), 2.83 (s, 3H), 2.01-1.86 (m, 2H), 1.77- 1.63 (m, 2H), 1.31-1.12 (m, 5H). |
| | (S)- or (R)-1-(1-(5H-pyrrolo[2,3- | |
| | b]pyrazin-2-yl)azepan-4-yl)-1-methyl- | |
| | 3-(1-methyl-2-oxo-5-(trifluoromethyl)- | |
| | 1,2-dihydropyridin-3-yl)urea | |

^{a}Separated by chiral SFC (Method A), first eluting isomer;
^{b}Separated by chiral SFC (Method A), second eluting isomer;
^{c}Separated by chiral SFC (Method B), first eluting isomer;
^{d}Separated by chiral SFC (Method B), second eluting isomer;
^{e}Separated by chiral SFC (Method D), first eluting isomer;
^{f}Separated by chiral SFC (Method D), second eluting isomer;
*Absolute stereochemistry not determined.

### Example 28: 1-methyl-1-(1-(3-methyl-1H-pyrazolo[3,4-b]pyridin-5-yl)piperidin-4-yl)-3-(1-methyl-2-oxo-5-(trifluoromethyl)-1,2-dihydropyridin-3-yl)urea

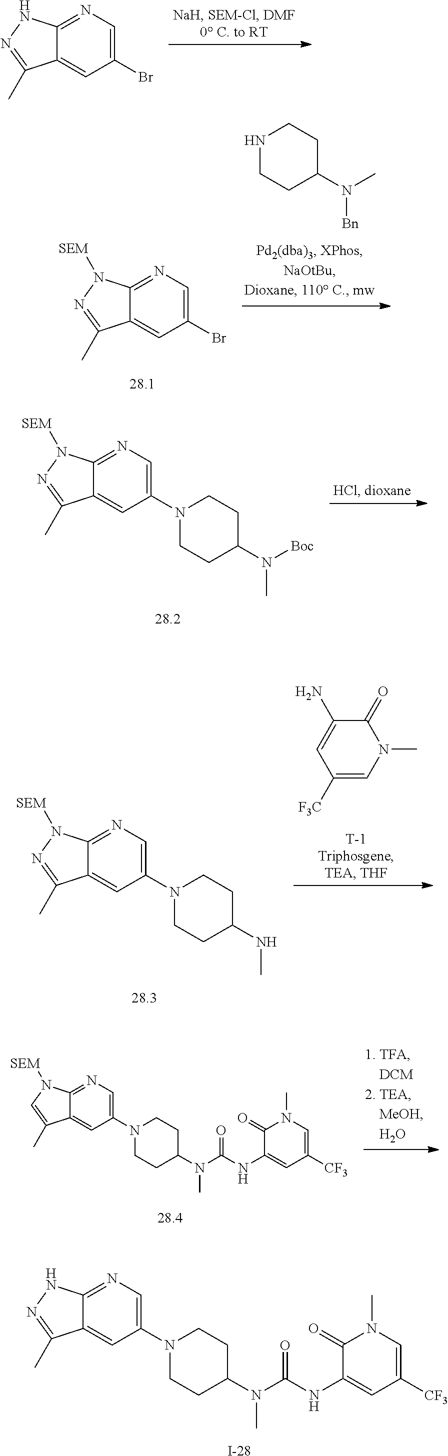

Synthesis of compound 28.1. To a solution of 5-bromo-3-methyl-1H-pyrazolo[3,4-b]pyridine (1.0 g, 4.72 mmol, 1.0 equiv) in DMF (20 mL) was added sodium hydride (0.283 g, 5.67 mmol, 1.5 equiv) at 0° C. and stirred for 30 min. To the mixture was added 2-(trimethylsilyl) ethoxymethyl chloride (1.26 g, 9.43 mmol, 2.0 equiv). The reaction mixture was stirred at room temperature for 2 h. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 7.7% ethyl acetate in hexane) to afford 28.1. MS(ES): m/z 342.31 [M+H]⁺.

Synthesis of compound 28.2. A mixture of 28.1 (0.800 g, 2.34 mmol, 1.0 equiv), 1.2 (0.500 g, 2.38 mmol, 1.1 equiv), and sodium tert-butoxide (0.561 g, 5.84 mmol 2.5 equiv) in 1,4-dioxane (10 mL) was degassed by bubbling through a stream of argon for 10 min. XPhos (0.222 g, 0.467 mmol, 0.2 equiv) and tris(diphenylideneacetone)dipalladium(O) (0.214 g, 0.233 mmol, 0.1 equiv) were added and degassed with argon for another 10 min. The reaction mixture was stirred at 110° C. in a microwave reactor for 1 h. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford 28.2 (0.650 g, 58.5%). MS(ES): m/z 475.71 [M+H]⁺.

Synthesis of compound 28.3. Compound 28.2 (0.650 g, 1.366 mmol, 1.0 equiv) was stirred with 4 M hydrochloric acid in dioxane (15 mL) at room temperature for 1.5 h. The reaction mixture was concentrated under reduced pressure to afford 28.3 (0.325 g, 63%). It was used in the next step without purification. MS (ES): m/z 375.59 [M+H]⁺.

Synthesis of compound 28.4. Compound 28.4 was prepared from compound 28.3 and T-1, following the procedure described in the synthesis of I-9. The product was purified by flash column chromatography on silica gel (CombiFlash®, 3.5% methanol in DCM). MS (ES): m/z 593.77 [M+H]⁺.

Synthesis of compound 1-28. Compound 1-28 was prepared from compound 28.4 following the procedure described in the synthesis of I-11. The product was purified by flash column chromatography on silica gel (CombiFlash®, 4.7% methanol in DCM). MS(ES): 464.39 m/z [M+H]⁺; ¹H NMR (DMSO-d₆, 400 MHz): δ 12.93 (s, 1H), 8.42 (s, 1H), 8.19 (s, 1H), 8.05 (s, 1H), 7.93 (s, 1H), 5.77 (s, 1H), 3.68-3.29 (m, 4H), 2.93 (s, 3H), 2.78 (s, 3H), 2.52-2.47 (m, 4H), 1.95-1.90 (m, 2H), 1.73-1.70 (m, 2H).

### Example 29: (R)-1-(3,3-difluoro-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and (S)-1-(3,3-difluoro-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

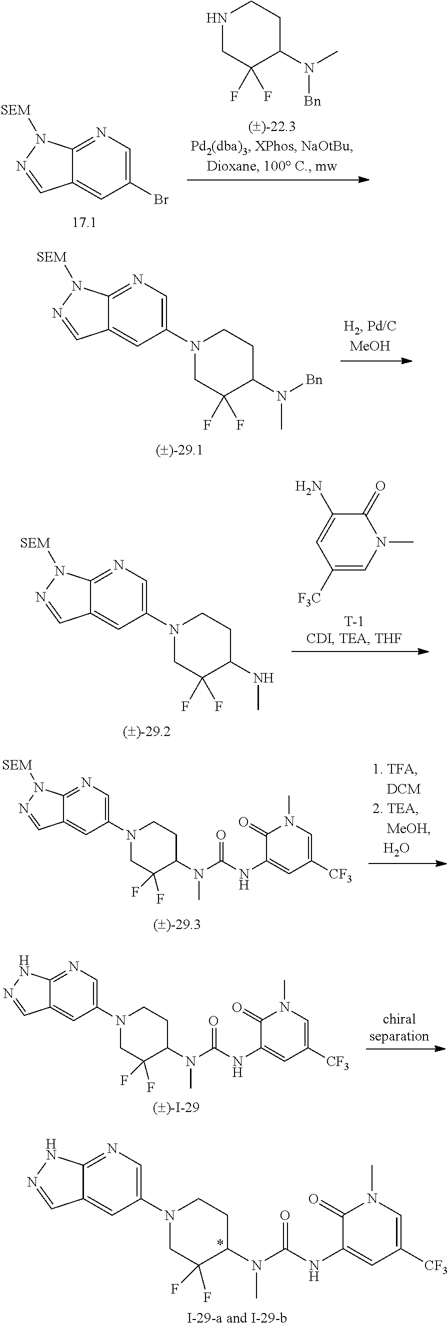

Synthesis of compound (±)-29.1. Compound (±)-29.1 was prepared from 17.1 and (±)-22.3, following the procedure described in the synthesis of 11.2. The product was purified by flash column chromatography on silica gel (CombiFlash®, 20% ethyl acetate in hexane). MS(ES): m/z 489 [M+H]⁺.

Synthesis of compound (±)-29.2. A mixture of (±)-29.1 (1.0 g, 2.05 mmol, 1.0 equiv) and 10% palladium on charcoal (0.500 g) in methanol (10 mL) was stirred under hydrogen (1 atm) for 16 h. The reaction mixture was filtered through a pad of Celite®. The filtrate was concentrated under reduced pressure to afford (±)-29.2 (0.700 g, 86%). MS(ES): m/z 398 [M+H]⁺.

Synthesis of compound (±)-29.3. Compound (±)-29.3 was prepared from (±)-29.2 and T-1, following the procedure described in the synthesis of I-12. The product was purified by flash column chromatography on silica gel (CombiFlash®, 5% methanol in DCM). MS(ES): m/z 616 [M+1]⁺.

Synthesis of I-29-a and I-29-b. Compound (±)-29 was prepared from compound (f)-29.3 following the procedure described in the synthesis of I-11. The product was purified by flash column chromatography on silica gel (CombiFlash®, 5% methanol in DCM). The racemate was separated by chiral HPLC (column: CHIRALPAK IBN-5 (250 mm*4.6 mm, 5 μm); mobile phase: (A) 0.1% diethylamine in n-hexane, (B) 0.1% diethylamine in isopropanol: methanol (1:1); flow rate: 20 mL/min) to afford first eluting fraction (I-29-a) and second eluting fraction (I-29-b).

I-29-a: MS(ES): m/z: 486.39 [M+H]⁺; ¹H NMR (DMSO-d₆, 400 MHz): δ 13.47 (s, 1H), 8.50 (m, 1H), 8.17 (s, 1H), 8.08 (s, 2H), 8.01 (s, 1H), 7.74 (s, 1H), 4.86-4.76 (m, 1H), 3.96-3.90 (m, 1H), 3.80-3.77 (m, 1H), 3.62 (s, 3H), 3.27 (m, 2H), 3.06 (s, 3H), 2.30 (m, 1H), 1.88 (m, 1H).

I-29-b: MS(ES): m/z: 486.36 [M+H]⁺; ¹H NMR (DMSO-d₆, 400 MHz): δ 13.47 (s, 1H), 8.50 (m, 1H), 8.17 (s, 1H), 8.08 (s, 2H), 8.01 (s, 1H), 7.74 (s, 1H), 4.86-4.76 (m, 1H), 3.96-3.90 (m, 1H), 3.80-3.77 (m, 1H), 3.62 (s, 3H), 3.27 (m, 2H), 3.06 (s, 3H), 2.30 (m, 1H), 1.88 (m, 1H).

### Example 30: 1-methyl-3-(1-methyl-2-oxo-5-(trifluoromethyl)-1,2-dihydropyridin-3-yl)-1-(1-(7-methyl-5H-pyrrolo[2,3-b]pyrazin-2-yl)piperidin-4-yl)urea

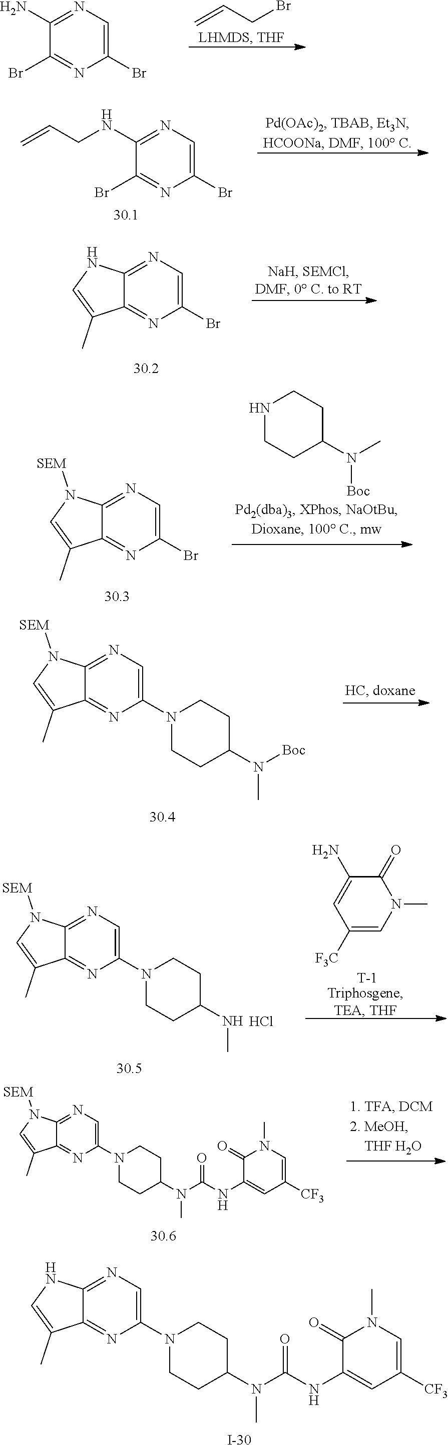

Synthesis of compound 30.1. To a solution of 3,5-dibromopyrazin-2-amine (29 g, 114.2 mmol, 1.0 equiv) in THE (100 mL) was added a solution of lithium bis(trimethylsilyl)amide (1 M in THF, 140 mL, 142.0 mmol, 1.2 equiv) at room temperature and stirred for 2 h. To the solution was added allyl bromide (27.9 g, 229.0 mmol, 2.0 equiv) dropwise and stirred for 16 h. The reaction mixture was slowly quenched with an aqueous ammonium chloride solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.0% ethyl acetate in hexane) to afford 30.1 (18 g, 54%). MS (ES): m/z 292.96 [M+H]⁺.

Synthesis of compound 30.2. A mixture of 30.1 (6 g, 3.1 mmol, 1 equiv), triethylamine (5.188 g, 42.8 mmol, 2.5 equiv), sodium formate (0.279 g, 4.10 mmol, 0.2 equiv), palladium(II) acetate (0.460 g, 2.0 mmol, 0.1 equiv) and tetrabutylammonium bromide (0.992 g, 3.0 mmol, 0.15 equiv) in DMF (6 mL) was purged with argon. The flask was sealed and reaction mixture was stirred at 100° C. for 16 h. It was cooled to room temperature, poured into water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 11% ethyl acetate in hexane) to afford 30.2 (0.620 g, 14%). MS (ES): m/z 212.05 [M+H]⁺.

Synthesis of compound 30.3. To a solution of 30.2 (0.620 g, 2.9 mmol, 1.0 equiv) in DMF (8 mL) was added sodium hydride (0.421 g. 8.77 mmol, 3 equiv) at 0° C. and stirred 45 min. To the mixture was added 2-(trimethylsilyl)ethoxymethyl chloride (0.728 g, 4.3 mmol, 1.5 equiv) at 0° C. and the resulting mixture was stirred at room temperature for 1 h. It was slowly poured into cold water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford 30.3 (0.450 g, 45%). MS (ES): m/z 342.31 [M+H]⁺.

Synthesis of compound 30.4. Compound 30.4 was prepared from compound 30.3 and tert-butyl methyl(piperidin-4-yl)carbamate, following the procedure described in the synthesis of 11.2. The product was purified by flash column chromatography on silica gel (CombiFlash®, 13% ethyl acetate in hexane). MS(ES): m/z 475.71 [M+H]⁺.

Synthesis of compound 30.5. Compound 30.4 (0.38 g, 0.64 mmol, 1 equiv) was stirred with 4 M hydrogen chloride in dioxane (5 mL) at room temperature for 1.5 h. It was concentrated under reduced pressure to afford 30.5 (0.3 g, 100%). It was used in the next step without purification. MS(ES): m/z 357.59 [M+H]⁺.

Synthesis of compound 30.6. Compound 30.6 was prepared from compound 30.5 and T-1, following the procedure described in the synthesis of I-9. The product was purified by flash column chromatography on silica gel (CombiFlash®, 2% methanol in DCM). MS(ES): m/z 593.66 [M+H]⁺.

Synthesis of compound 1-30. Compound 1-30 was prepared from compound 30.6 following the procedure described in the synthesis of I-11. The product was purified by flash column chromatography on silica gel (CombiFlash®, 2.2% methanol in DCM). MS(ES): m/z 464.28 [M+H]⁺; ¹H NMR (DMSO-d₆, 400 MHz): 9.18 (s, 1H), 8.18 (s, 1H), 8.04 (s, 1H), 7.90 (s, 1H), 7.10 (s, 1H), 7.37 (s, 1H), 4.46-4.38 (m, 2H), 4.27 (m, 1H), 4.09 (m, 1H), 3.59 (s, 3H), 2.88-2.82 (m, 4H), 2.19 (s, 3H), 1.79-1.65 (m, 2H), 1.54 (m, 2H).

### Example 31: 1-methyl-3-(1-methyl-2-oxo-5-(trifluoromethyl)-1,2-dihydropyridin-3-yl)-1-(1-(6-methyl-5H-pyrrolo[2,3-b]pyrazin-2-yl)piperidin-4-yl)urea

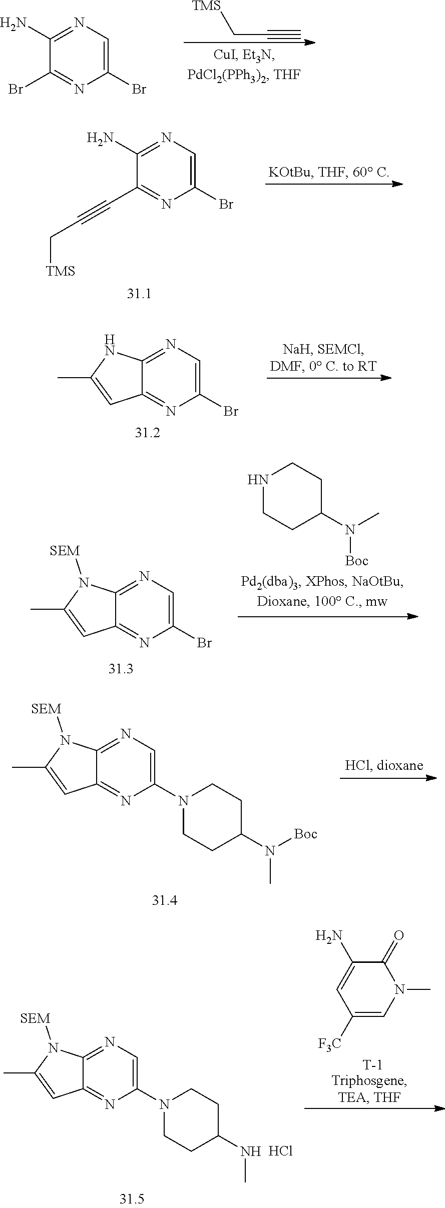

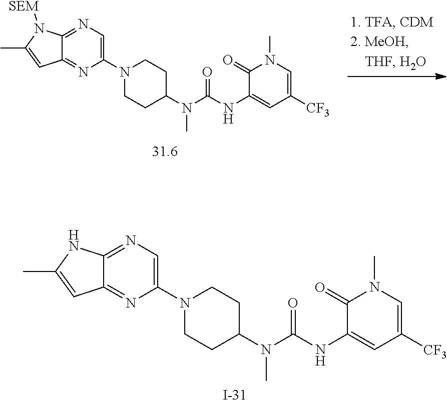

Synthesis of compound 31.1. A mixture of 3,5-dibromopyrazin-2-amine (3 g, 11.6 mmol, 1.0 equiv), triethylamine (1.43 g, 14.24 mmol, 1.2 equiv), copper iodide (0.113 g, 0.59 mmol, 0.05 equiv) and 1,1-bis(triphenylphosphine)palladium(II) chloride (0.417 g, 0.59 mmol, 0.05 equiv) in THF (40 mL) was degassed by bubbling through a stream of argon for 10 min. Trimethyl(prop-2-yn-1-yl)silane (1.6 g, 14.24 mmol, 1.2 equiv) was added and the reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in hexane) to afford 31.1 (3.0 g, 89%). MS(ES): m/z 286.12 [M+2]⁺.

Synthesis of compound 31.2. To a solution of 31.1 (3 g, 10.55 mmol, 1.0 equiv) in THF (40 mL) was added 1 M potassium tert-butoxide in THF (21.12 mL, 21.12 mmol, 2.0 equiv). The reaction mixture was stirred at 60° C. for 2 h. It was diluted with ethyl acetate, washed with water, and then brine. The organic layer was dried over anhydrous sodium sulfate, filtered, and concentrated under reduced pressure to afford 31.2 (1.1 g, 49%). It was used in the next step without purification. MS(ES): m/z 214.23 [M+2]⁺.

Synthesis of compound 31.3. To a solution of 31.2 (1.1 g, 5.19 mmol, 1.0 equiv) in DMF (5 mL) at 0° C. was added 60% sodium hydride (0.311 g, 7.78 mmol, 1.0 equiv) and stirred for 15 min. To the mixture was added 2-(trimethylsilyl)ethoxymethyl chloride (1.03 g, 6.22 mmol, 1.2 equiv) at 0° C. and the resulting mixture was stirred at room temperature for 2 h. The reaction mixture was poured into crushed ice-water and extracted with ethyl acetate. The combined organic layers were washed with brine, dried over anhydrous sodium sulfate, filtered, and concentrated under reduced pressure. The residue was purified by silica gel flash column chromatography on silica gel (CombiFlash®, 10% ethyl acetate in hexane) to afford 31.3. MS(ES): m/z 344.31 [M+2]⁺.

Synthesis of compound 31.4. Compound 31.4 was prepared from compound 31.3 and tert-butyl methyl(piperidin-4-yl)carbamate, following the procedure described in the synthesis of 11.2. The product was purified by flash column chromatography on silica gel (CombiFlash®, 34% ethyl acetate in hexane). MS(ES): m/z: 476.52 [M+H]⁺.

Synthesis of compound 31.5. To a solution of 31.4 (0.15 g, 0.31 mmol, 1.0 equiv) in DCM (10 mL) was added 4 M HCl in dioxane (5 mL) at 0° C. and reaction mixture was stirred at room temperature for 1 h. It was concentrated under reduced pressure to afford 31.5 (0.12 g, 92%). It was used in the next step without purification. MS(ES): m/z 376.48 [M+H]⁺.

Synthesis of compound 31.6. Compound 31.6 was prepared from compound 31.5 and T-1, following the procedure described in the synthesis of I-9. The product was purified by silica gel flash column chromatography on silica gel (CombiFlash®, 1.5% methanol in DCM). MS(ES): m/z 593.73 [M+H]⁺.

Synthesis of 1-31. Compound 1-31 was prepared from compound 31.6 following the procedure described in the synthesis of I-11. The product was purified by flash column chromatography on silica gel (CombiFlash®, 3.3% methanol in DCM). MS(ES): m/z: 464.40 [M+H]⁺; ¹H NMR (DMSO-d₆, 400 MHz): δ 11.39 (s, 1H), 8.17 (s, 1H), 8.04 (s, 1H), 7.90 (s, 1H), 7.89 (s, 1H), 6.05 (s, 1H), 4.35-4.26 (m, 3H), 3.59 (s, 3H), 2.86-2.81 (m, 5H), 2.39 (s, 3H), 1.80-1.63 (m, 4H).

### Example 34: ((R)—N-(5-(3,3-difluoro-4-(1-methyl-3-(1-methyl-2-oxo-5-(trifluoromethyl)-1,2-dihydropyridin-3-yl)ureido)piperidin-1-yl)pyrazin-2-yl)cyclopropanecarboxamide and (S)—N-(5-(3,3-difluoro-4-(1-methyl-3-(1-methyl-2-oxo-5-(trifluoromethyl)-1,2-dihydropyridin-3-yl)ureido)piperidin-1-yl)pyrazin-2-yl)cyclopropanecarboxamide

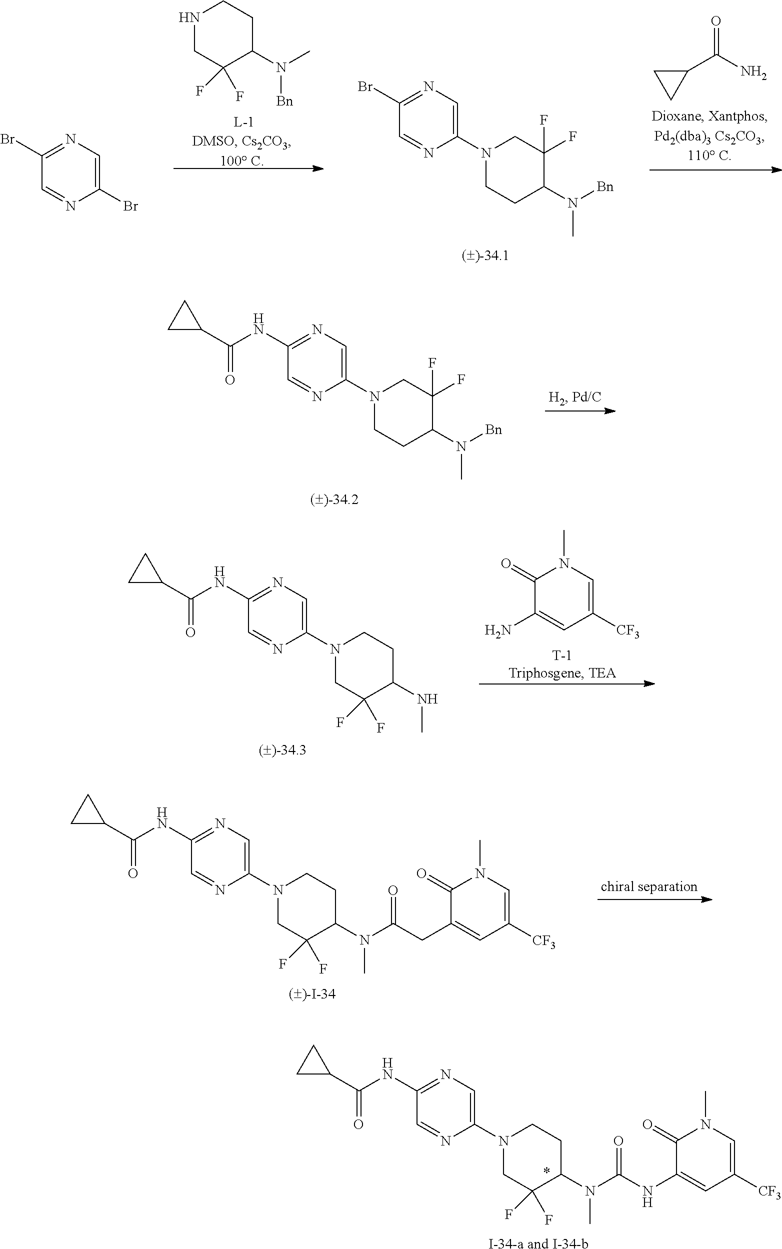

Synthesis of compound (±)-34.1. A mixture of 2,5-dibromopyrazine (1.2 g, 5.04 mmol, 1.0 equiv), cesium carbonate (3.2 g, 10.08 mmol, 2.0 equiv) and L-1 (1.2 g, 5.04 mmol, 1.0 equiv) in dimethyl sulfoxide (10 mL) was stirred at 100° C. for 5 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (20% ethyl acetate in hexane) to afford (±)-34.1 (0.68 g, 34%). MS(ES): m/z: 398.08 [M+H]⁺.

Synthesis of compound (±)-34.2. A mixture of (±)-34.2 (0.68 g, 1.71 mmol, 1.0 equiv), cyclopropanecarboxamide (0.728 g, 8.56 mmol, 5.0 equiv), 4,5-bis(diphenylphosphino)-9,9-dimethylxanthene (0.198 g, 0.342 mmol, 0.2 equiv) and cesium carbonate (1.67 g, 5.13 mmol, 3.0 equiv) in 1,4-dioxane (5 mL) was purged with argon for 5 min. To the mixture was added tris(dibenzylideneacetone)dipalladium(O) (0.156 g, 0.171 mmol, 0.1 equiv) and purged with argon for 10 min. The reaction mixture was stirred at 110° C. for 16 h. It was cooled to room temperature, transferred into water and extracted with ethyl acetate. The organic layer was separated, dried over anhydrous sodium sulfate, filtered, and concentrated under reduced pressure. The residue was purified by flash column chromatography on silica gel (CombiFlash®, 29% ethyl acetate in hexane) to afford (±)-34.2 (0.495 g, 82%). MS(ES): m/z: 402.20 [M+H]⁺.

Synthesis of compound (±)-34.3. A mixture of (±)-34.2 (0.495 g, 1.23 mmol, 1.0 equiv) and palladium on charcoal (10 wt %, 0.15 g) in methanol (3.0 mL) was stirred under hydrogen atmosphere (1 atm) at room temperature for 3 h. It was filtered through a pad of Celite® and rinsed with diethyl ether. The filtrate was concentrated under reduced pressure to afford (±)-34.3 (0.25 g, 65%). MS(ES): m/z 312.16 [M+H]⁺.

Synthesis of compound (±)-I-34. Compound (±)-I-33 was prepared from compound (±)-34.3 and T-1, following the procedure described in the synthesis of I-9. The product was purified by column chromatography on silica gel (2.5% methanol in DCM). MS(ES): m/z 530.19 [M+H]⁺.

I-34-a and I-34-b: The racemate was separated by chiral SFC (Method A) to afford first eluting fraction (I-34-a) and second eluting fraction (I-34-b). (*Absolute configuration was not determined.)

I-34-a: MS(ES): m/z: 530.45 [M+H]⁺, ¹H-NMR (400 MHz, DMSO-d₆): δ 10.67 (s, 1H), 8.79 (s, 1H), 8.25 (s, 1H), 8.16 (s, 1H), 8.08 (s, 1H), 7.60 (s, 1H), 4.95-4.86 (m, 1H), 4.71-4.65 (m, 1H), 4.46-4.43 (m, 1H), 3.60 (s, 3H), 3.19-3.13 (m, 1H), 2.97 (s, 3H), 2.14-2.10 (m, 1H), 1.95-1.93 (m, 2H), 1.85-1.82 (m, 1H), 0.81-0.79 (m, 4H).

I-34-b: MS(ES): m/z: 530.45 [M+H]⁺, ¹H-NMR (400 MHz, DMSO-d₆): δ 10.67 (s, 1H), 8.79 (s, 1H), 8.25 (s, 1H), 8.16 (s, 1H), 8.08 (s, 1H), 8.05 (s, 1H), 4.96-4.86 (m, 1H), 4.71-4.65 (m, 1H), 4.46-4.43 (m, 1H), 3.60 (s, 3H), 3.19-3.13 (m, 1H), 2.97 (s, 3H), 2.17-2.10 (m, 1H), 1.99-1.95 (m, 2H), 1.85-1.82 (m, 1H), 0.81-0.79 (m, 4H).

### Example 35: (R)—N-(5-(3,3-difluoro-4-(1-methyl-3-(1-methyl-2-oxo-5-(trifluoromethyl)-1,2-dihydropyridin-3-yl)ureido)piperidin-1-yl)pyrazin-2-yl)acetamide and (S)—N-(5-(3,3-difluoro-4-(1-methyl-3-(1-methyl-2-oxo-5-(trifluoromethyl)-1,2-dihydropyridin-3-yl)ureido)piperidin-1-yl)pyrazin-2-yl)acetamide

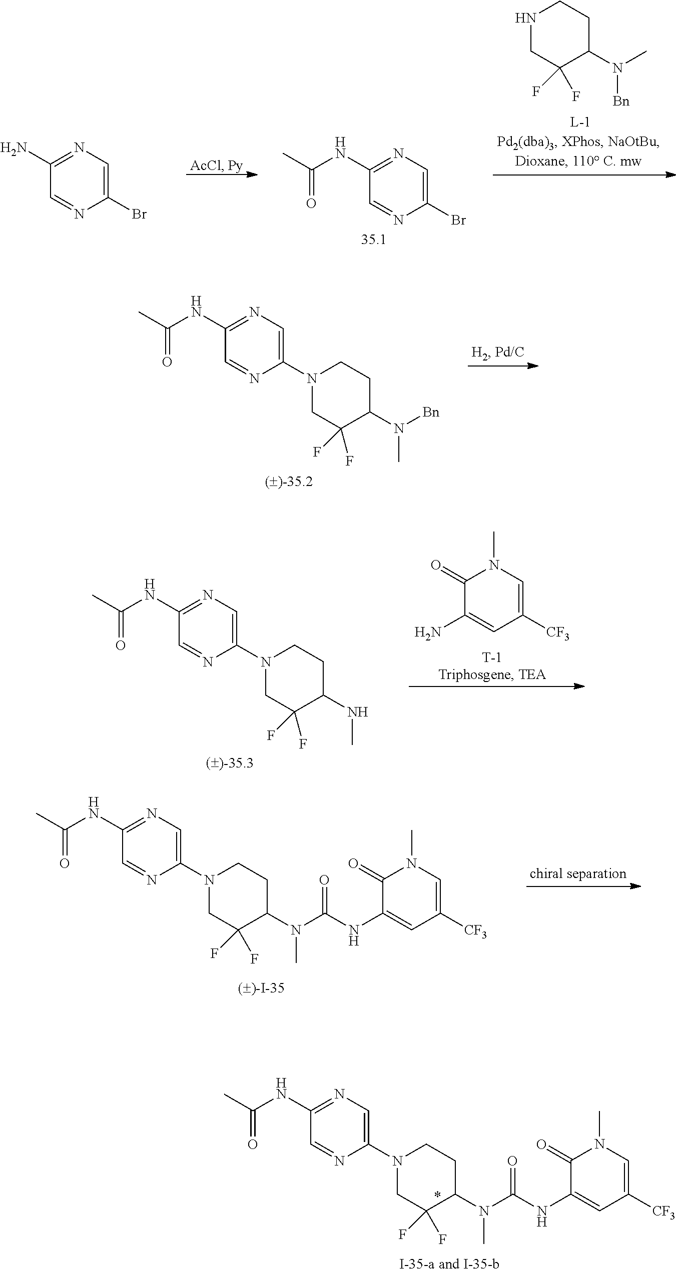

Synthesis of compound 35.1. To a solution of 5-bromopyrazin-2-amine (1.0 g, 5.74 mmol, 1 equiv) and pyridine (11.1 mL, 139.67 mmol, 24 equiv) in DCM (11 mL) at 0° C. was added acetyl chloride (0.66 mL, 9.195 mmol, 1.6 equiv). The reaction mixture was stirred for 1 h. It was concentrated under reduced pressure. The residue was purified by flash column chromatography on silica gel (CombiFlash®, 20% ethyl acetate in hexane) to afford 35.1 (0.87 g, 70%). MS(ES): m/z: 217.04 [M+H]⁺.

Synthesis of compound (±)-35.2. Compound (±)-35.2 was prepared from 35.1 and L-1, following the procedure described in the synthesis of compound 11.1. The product was purified by flash column chromatography on silica gel (CombiFlash®, 15% ethyl acetate in hexane). MS(ES): m/z: 376.19 [M+H]⁺.

Synthesis of compound (±)-35.3. A mixture of (±)-35.2 (0.31 g, 0.82 mmol, 1.0 equiv) and 10% palladium on carbon (0.5 g) in methanol (3.0 mL) was stirred under hydrogen atmosphere (1 atm) for 4 h. The reaction mixture was filtered through a pad of Celite® and rinsed with methanol. The filtrate was concentrated under reduced pressure to afford (±)-35.3 (0.205 g, 87%). MS(ES): m/z 286.14 [M+H]⁺.

Synthesis of compound (±)-I-35. Compound (±)-I-35 was prepared from compound (±)-34.3 and T-1, following the procedure described in the synthesis of I-9. The product was purified by flash column chromatography on silica gel (CombiFlash®, 2.9% methanol in DCM). MS(ES): m/z 504.17 [M+H]⁺.

I-35-a and I-35-b: The racemate was separated by chiral SFC (Method A) to afford first eluting fraction (I-35-a) and second eluting fraction (I-35-b). (*Absolute configuration was not determined.)

I-35-a: MS(ES): m/z: 504.4 [M+H]⁺, ¹H NMR (DMSO-d₆, 400 MHz): δ 10.38 (s, 1H), 8.78 (s, 1H), 8.25 (s, 1H), 8.16 (s, 1H), 8.08 (s, 1H), 8.05 (s, 1H), 4.99-4.83 (m, 1H), 4.61-4.77 (m, 2H), 4.41-4.49 (m, 1H), 3.59 (s, 3H), 3.21-3.11 (m, 1H), 2.97 (s, 3H), 2.19-2.07 (m, 1H), 2.06 (s, 3H), 1.89-1.76 (m, 1H).

I-35-b: MS(ES): m/z: 504.44 [M+H]⁺, ¹H NMR (DMSO-d₆, 400 MHz): δ 10.38 (s, 1H), 8.79 (s, 1H), 8.25 (s, 1H), 8.16 (s, 1H), 8.08 (s, 1H), 8.05 (s, 1H), 4.93-4.88 (m, 1H), 4.71-4.69 (m, 2H), 4.46-4.43 (m, 1H), 3.60 (s, 3H), 3.20-3.13 (m, 1H), 2.97 (s, 3H), 2.14-2.09 (m, 1H), 2.06 (s, 3H), 1.89-1.80 (m, 1H).

### Example 42: 1-methyl-3-(1-methyl-2-oxo-5-(trifluoromethyl)-1,2-dihydropyridin-3-yl)-1-(1-(7-(methylamino)pyrazolo[1,5-a]pyrimidin-3-yl)piperidin-4-yl)urea

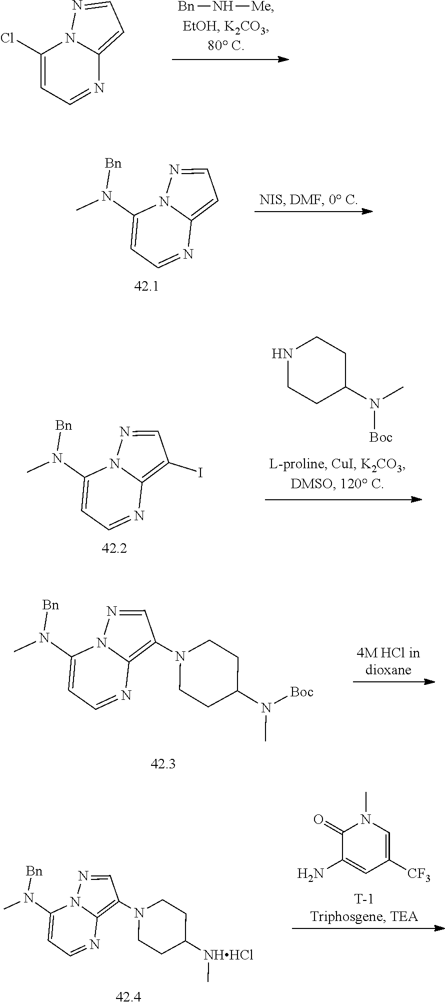

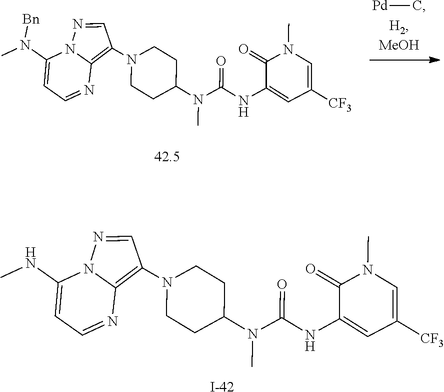

Synthesis of compound 42.1. A mixture of 7-chloropyrazolo[1,5-a]pyrimidine (1.0 g, 6.49 mmol, 1.0 equiv), potassium carbonate (2.6 g, 19.48 mmol 3.0 equiv) and N-methylbenzylamine (0.942 g, 7.78 mmol, 1.2 equiv) in ethanol (10 mL) was stirred at 80° C. for 4 h. It was cooled to rt and most solvent was removed under reduced pressure. The residue was transferred into ice-water. The precipitated solids were collected by filtration, washed with cold water and dried in vacuum to afford 42.1 (1.0 g, 64%). MS(ES): m/z: 239.12 [M+H]⁺.

Synthesis of compound 42.2. To a solution of 42.1 (1.0 g, 4.18 mmol, 1.0 equiv) in DMF (10 mL) was added N-iodosuccinimide (0.94 g, 4.18 mmol, 1.0 equiv) in portions at 0° C. The reaction mixture was stirred 20° C. for 2 h. It was transferred into ice-water. The solids precipitated were collected by filtration, washed with cold water and dried in vacuum to afford 42.2 (0.9 g, 59%). MS(ES): m/z 365.02 [M+H]⁺.

Synthesis of compound 42.3. A mixture of 42.2 (0.9 g, 6.18 mmol, 1.0 equiv), tert-butyl methyl(piperidin-4-yl)carbamate (2.6 g, 30.90 mmol, 5.0 equiv) and potassium carbonate (6.0 g, 18.54 mmol, 3.0 equiv) in DMSO (10 mL) was degassed by bubbling through a stream of argon for 10 min. L-Proline (0.451 g, 0.618 mmol, 0.1 equiv) and copper(I) iodide (0.2 g) were added and degassed for 5 min. The reaction mixture was stirred at 120° C. for 2 h in a microwave reactor. It was cooled to room temperature, reaction mixture was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 35% ethyl acetate in hexane) to afford 42.3 (0.6 g, 54%). MS(ES): m/z 451.27 [M+H]⁺.

Synthesis of compound 42.4. To a solution of 42.3 (0.6 g, 1.09 mmol, 1.0 equiv) in DCM (5.0 mL) was added hydrochloric acid (4.0 M in dioxane, 0.6 mL) at 0° C. The reaction mixture was stirred at room temperature for 3 h. It was concentrated under vacuum and directly used for next step 42.4 (0.4 g, 92%). MS(ES): m/z 351.27 [M+H]⁺.

Synthesis of compound 42.5. Compound 42.5 was prepared from compound 42.4 and T-1 following the procedure described in the synthesis of I-9. The product was purified by column chromatography on silica gel (2.5% methanol in DCM). MS(ES): m/z 569.25 [M+H]⁺.

Synthesis of compound 1-42. A mixture of 42.5 (0.105 g, 0.18 mmol, 1.0 equiv) and palladium on charcoal (10 wt %, 0.05 g) in methanol (3.0 mL) was stirred under hydrogen atmosphere (1 atm) at room temperature for 3 h. It was filtered through a pad of Celite®. The filtrate was concentrated under reduced pressure. The residue was washed with diethyl ether to afford 1-42 (0.030 g, 35%). MS(ES): m/z 479.21 [M+H]⁺. ¹H-NMR (400 MHz, DMSO-d₆): δ 8.18 (s, 1H), 8.03-8.02 (m, 2H), 7.91 (s, 1H), 7.83-7.81 (m, 2H), 5.98-5.95 (m, 1H), 4.19-4.12 (m, 1H), 3.91-3.89 (m, 2H), 3.60 (s, 3H), 2.97-2.95 (m, 3H), 2.92 (s, 3H), 2.71-2.65 (m, 2H), 1.96-1.88 (m, 2H), 1.66-1.63 (m, 2H).

### Example 43: 1-((1S,4S,5R)-2-(5H-pyrrolo[2,3-b]pyrazin-2-yl)-2-azabicyclo[2.2.1]heptan-5-yl)-1-methyl-3-(1-methyl-2-oxo-5-(trifluoromethyl)-1,2-dihydropyridin-3-yl)urea and 1-((1R,4R,5S)-2-(5H-pyrrolo[2,3-b]pyrazin-2-yl)-2-azabicyclo[2.2.1]heptan-5-yl)-1-methyl-3-(1-methyl-2-oxo-5-(trifluoromethyl)-1,2-dihydropyridin-3-yl)urea

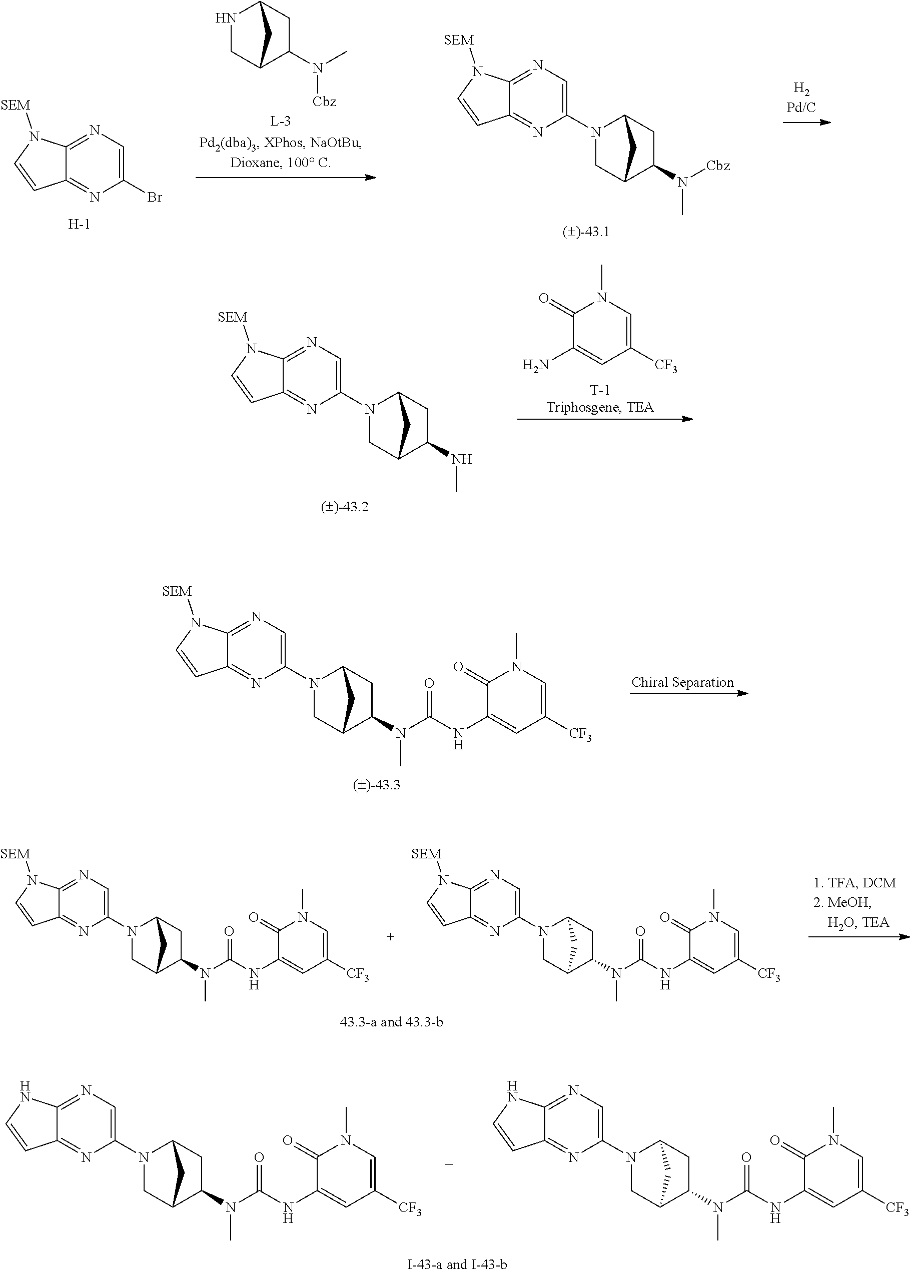

Synthesis of compound (±)-43.1. Compound (±)-43.1 was prepared from H-1 and L-3, following the procedure described in the synthesis of 11.1. The product was purified by flash column chromatography on silica gel (CombiFlash®, 25% ethyl acetate in hexane). MS(ES): m/z: 508.27 [M+H]⁺.

Synthesis of compound (±)-43.2. A mixture of compound (±)-43.1 (0.75 g, 0.62 mmol, 1.0 equiv) and 10% palladium on carbon (0.2 g) in methanol (5 mL) was stirred under hydrogen atmosphere (1 atm) for 5 h. The reaction mixture was filtered through a pad of Celite® and rinsed with methanol. The filtrate was concentrated under reduced pressure to afford (f)-43.2 (0.21 g, 89%). MS(ES): m/z 374.23 [M+H]⁺.

Synthesis of compound (±)-43.3. Compound (±)-43.3 was prepared from compound (±)-43.2 and T-1 following the procedure described in the synthesis of I-9. The product was purified by column chromatography on silica gel (1.5% methanol in DCM). MS(ES): m/z 520.26 [M+H]⁺.

Compound 43.3-a and 43.3-b: The racemate was separated by chiral SFC (Method B) to afford first eluting fraction (43.3-a), MS(ES): m/z: 592.26 [M+H]⁺, and second eluting fraction (43.3-b), MS(ES): m/z: 592.26 [M+H]⁺. (*Absolute configuration was not determined.)

Synthesis of I-43-a. To a solution of 43.3-a (0.075 g, 0.14 mmol, 1.0 equiv) in DCM (1.0 mL) was cooled to 0° C. and added trifluoroacetic acid (0.5 mL). The reaction mixture was stirred at room temperature for 0.5 h. Most solvent was removed under reduced pressure. The residue was diluted with DCM (3.0 mL), basified with saturated sodium bicarbonate solution, and extracted with DCM. The combined organic layers were washed with brine, dried over anhydrous sodium sulfate, filtered, and concentrated under reduced pressure. To the residue was added water (1.5 mL), triethylamine (1.5 mL) and methanol (1.5 mL) stirred for 16 h at room temperature. It was transferred into cold water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.5% methanol in DCM) to afford I-43-a (0.020 g, 30%). MS(ES): m/z 462.39 [M+H]⁺, ¹H-NMR (400 MHz, DMSO-d₆): δ 11.47 (s, 1H), 8.13-8.12 (d, J=2.4 Hz, 1H), 8.01 (s, 1H), 7.92 (s, 1H), 7.68 (s, 1H), 7.54-7.53 (t, J=6.0 Hz, 1H), 6.31-6.30 (m, 1H), 4.57 (s, 1H), 4.09-4.06 (m, 1H), 3.56 (s, 3H), 3.51-3.48 (m, 1H), 3.21-3.19 (m, 1H), 2.98 (s, 3H), 2.75-2.78 (m, 1H), 2.21-2.29 (m, 1H), 1.91-1.89 (m, 1H), 1.82-1.74 (2H).

Synthesis of 1-43-b. Compound 1-43-b was prepared from compound 43.3-b following the procedure described in the synthesis of I-43-a. MS(ES): m/z 462.32 [M+H]⁺, 1H-NMR (400 MHz, DMSO-d₆): δ 11.47 (s, 1H), 8.13 (s, 1H), 8.01 (s, 1H), 7.92 (s, 1H), 7.68 (s, 1H), 7.53 (s, 1H), 6.30 (s, 1H), 4.57 (s, 1H), 4.05-4.09 (m, 1H), 3.56 (s, 3H), 3.51-3.49 (m, 1H), 3.21-3.19 (m, 1H), 2.98 (s, 3H), 2.78-2.76 (m, 1H), 2.28-2.22 (m, 1H), 1.91-1.89 (m, 1H), 1.83-1.73 (m, 2H).

### Example 44: N-(5-((1S,4S,5R)-5-(1-methyl-3-(1-methyl-2-oxo-5-(trifluoromethyl)-1,2-dihydropyridin-3-yl)ureido)-2-azabicyclo[2.2.1]heptan-2-yl)pyrazin-2-yl)cyclopropanecarboxamide and N-(5-((1R,4R,5S)-5-(1-methyl-3-(1-methyl-2-oxo-5-(trifluoromethyl)-1,2-dihydropyridin-3-yl)ureido)-2-azabicyclo[2.2.1]heptan-2-yl)pyrazin-2-yl)cyclopropanecarboxamide

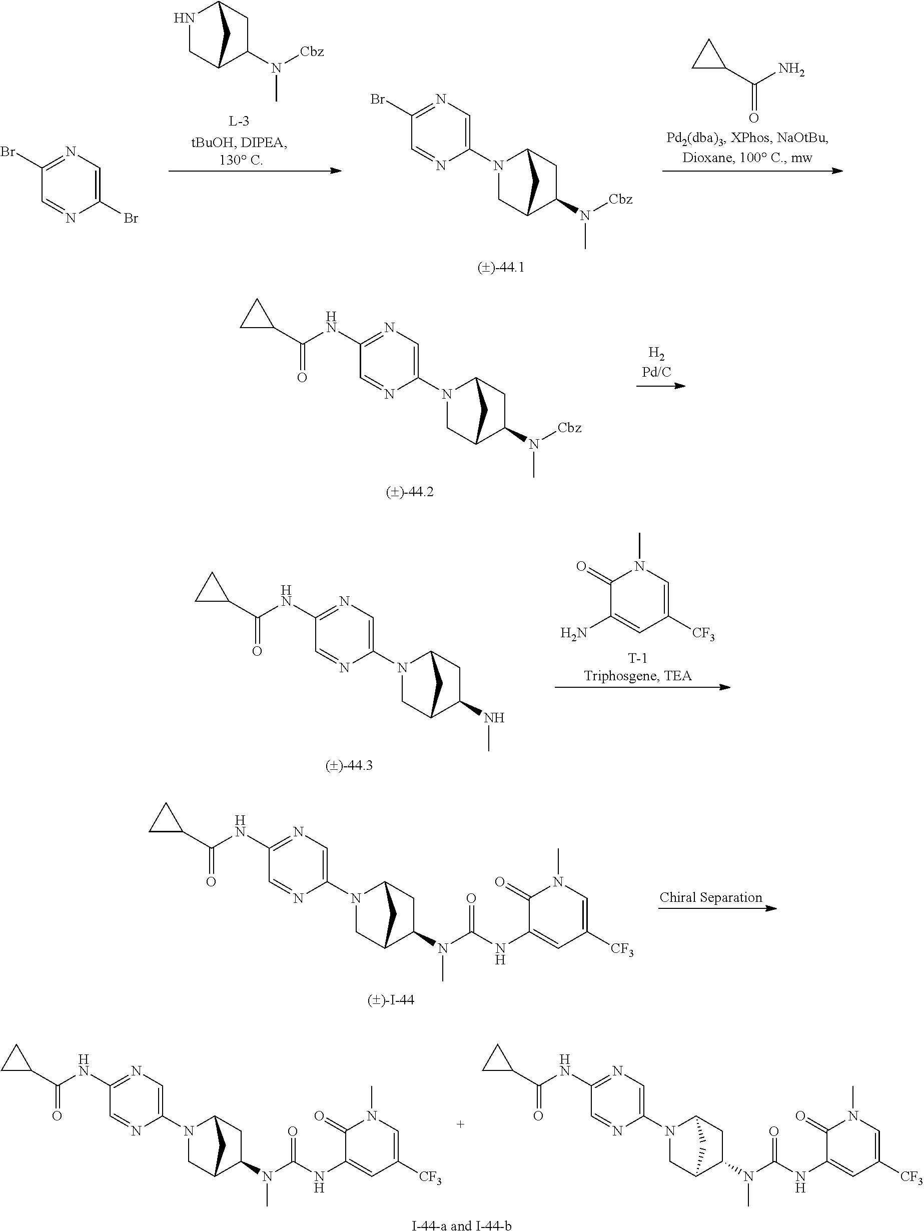

Synthesis of compound (±)-44.1. A solution of 2,5-dibromopyrazine (0.5 g, 2.109 mmol, 1.0 equiv), L-3 (0.548 g, 2.109 mmol, 1.0 equiv) and N,N′-diisopropylethylamine (1.16 mL, 6.32 mmol, 3 equiv) in t-butanol (5 mL) was stirred in a microwave reactor at 130° C. for 2 h. It was concentrated under reduced pressure. The residue was purified by flash column chromatography on silica gel (CombiFlash®, 29% ethyl acetate in hexane) to afford (±)-44.1 (0.504 g, 57%), MS(ES): m/z 418.08 [M+H]⁺.

Synthesis of compound (±)-44.2. A mixture of (±)-44.1 (0.5 g, 1.199 mmol, 1.0 equiv), cyclopropanecarboxamide (0.305 g, 3.597 mmol, 3.0 equiv) and sodium tert-butoxide (0.345 g, 3.597 mmol, 3.0 equiv) in 1,4-dioxane (5.0 mL) was degassed by bubbling through a stream of argon for 10 min. Tris(dibenzylideneacetone)dipalladium(O) (0.054 g, 0.0599 mmol, 0.05 equiv) and 2-dicyclohexyl phosphino-2,4,6-triisopropylbiphenyl (0.114 g, 0.239 mmol, 0.2 equiv) were added and degassed by bubbling through a stream of argon for 5 min. The reaction mixture was stirred at 100° C. for 1 h in a microwave reactor. It was cooled to room temperature, and filtered through a pad of Celite®. The filtrate was transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 45% ethyl acetate in hexane) to afford (f)-44.2 (0.405 g, 80%), MS(ES): m/z 422.21 [M+H]⁺.

Synthesis of compound (±)-44.3. A mixture of compound (±)-44.2 (0.405 g, 0.95 mmol, 1.0 equiv) and 10% palladium on carbon (0.12 g) in methanol (2.0 mL) was stirred under hydrogen atmosphere (1 atm) for 1 h. The reaction mixture was filtered through a pad of Celite® and rinsed with methanol. The filtrate was concentrated under reduced pressure to afford (f)-44.3 (0.18 g, 65%). MS(ES): m/z 288.17 [M+H]⁺.

Synthesis of compound (±)-I-44. Compound (±)-I-44 was prepared from compound (±)-44.3 and T-1 following the procedure described in the synthesis of I-9. The product was purified by flash column chromatography on silica gel (3.1% methanol in DCM). MS(ES): m/z 506.20 [M+H]⁺.

I-44-a and I-44-b: The racemate was separated by chiral SFC (Method H) to afford first eluting fraction (I-44-a) and second eluting fraction (I-44-b). (*Absolute configuration was not determined.)

I-44-a: MS(ES): m/z: 506.42 [M+H]⁺, ¹H-NMR (400 MHz, DMSO-d₆): δ 10.49 (s, 1H), 8.70 (s, 1H), 8.12 (s, 1H), 8.01 (s, 1H), 7.91 (s, 1H), 7.76 (s, 1H), 4.07-4.11 (m, 1H), 3.57 (s, 3H), 3.43-3.33 (m, 1H), 3.14-3.12 (m, 1H), 2.95 (s, 3H), 2.75-2.72 (m, 1H), 2.20-2.15 (m, 2H), 1.93-1.85 (m, 2H), 1.77-1.69 (m, 2H), 0.82-0.76 (m, 4H).

I-44-b: MS(ES): m/z 506.2, ¹H-NMR (400 MHz, DMSO-d₆): δ 10.48 (s, 1H), 8.70 (s, 1H), 8.11 (s, 1H), 8.00 (s, 1H), 7.91 (s, 1H), 7.75 (s, 1H), 4.07-4.11 (m, 1H), 3.56 (s, 3H), 3.44-3.32 (m, 1H), 3.14-3.11 (m, 1H), 2.95 (s, 3H), 2.75-2.72 (m, 1H), 2.20-2.15 (m, 2H), 1.93-1.85 (m, 2H), 1.77-1.69 (m, 2H), 0.81-0.76 (m, 4H).

### Example 45: (S)-1-(3,3-dimethyl-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and (R)-1-(3,3-dimethyl-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

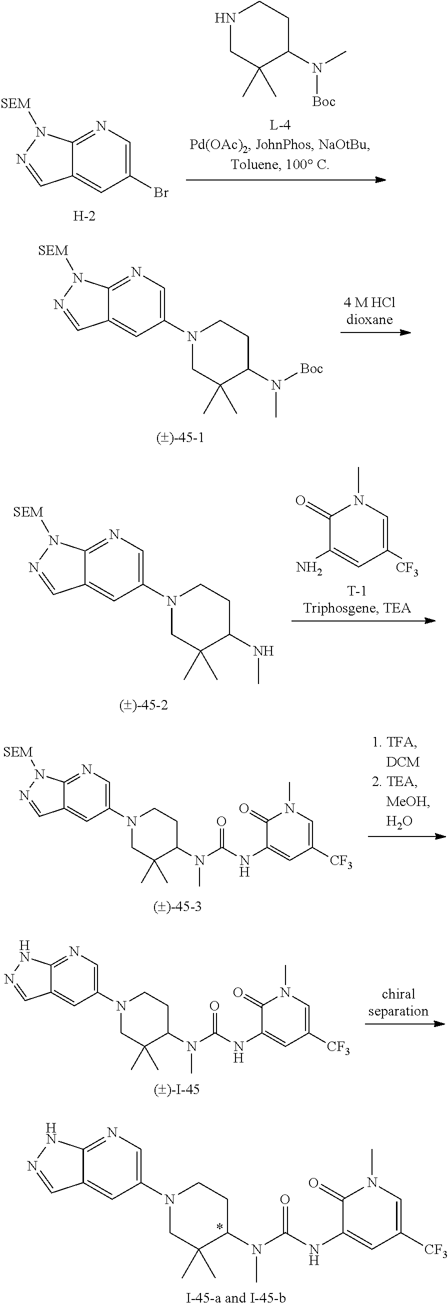

Synthesis of compound (±)-45-1. A mixture of H-2 (0.465 g, 1.42 mmol, 1.0 equiv), L-4 (0.550 g, 2.27 mmol, 1.6 equiv) and sodium tert-butoxide (0.272 g, 2.84 mmol, 2.0 equiv) in toluene (6.0 mL) was purged with a stream of argon for 15 min. To the mixture was added (2-biphenyl)di-tert-butylphosphine, (0.042 g, 0.142 mmol, 0.1 equiv) and palladium(II) acetate (0.031 g, 0.142 mmol, 0.1 equiv) and degassed for 10 min. The reaction mixture was stirred at 100° C. for 5 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 22% ethyl acetate in hexane) to afford (±)-45-1 (0.330 g, 42%). MS(ES): m/z 490.31 [M+H]⁺.

Synthesis of compound (±)-45-2. To a solution of (±)-45-1 (0.33 g, 67.48 mmol, 1.0 equiv) in DCM (0.33 mL) was added 4.0 M hydrochloric acid in dioxane (0.33 mL). The reaction mixture was stirred at room temperature for 3 h. The reaction mixture was concentrated under reduced pressure and azeotroped with DCM three times to afford (±)-45-2 (0.26 g, 99%). MS(ES): m/z 390.26 [M+H]⁺.

Synthesis of compound (±)-45-3. Compound (±)-45-3 was prepared from compound (±)-45-2 and T-1 following the procedure described in the synthesis of I-9. The product was purified by flash column chromatography on silica gel (3.0% methanol in DCM). MS(ES): m/z 608.26 [M+H]⁺.

Synthesis of compound (±)-45. Compound (±)-45 was prepared from compound (f)-45-3 following the procedure described in the synthesis of I-11. The product was purified by flash column chromatography on silica gel (CombiFlash®, 3.2% methanol in DCM). MS(ES): m/z 478.21 [M+H]⁺.

I-45-a and I-45-b: The racemate was separated by chiral HPLC (Method L) to afford first eluting fraction (I-45-a) and second eluting fraction (I-45-b). (*Absolute configuration was not determined.)

I-45-a: MS(ES): m/z: 478.43 [M+H]⁺, ¹H-NMR (400 MHz, DMSO-d₆): δ 13.36 (s, 1H), 8.41 (s, 1H), 8.18 (s, 1H), 8.02 (s, 1H), 7.97 (s, 1H), 7.95 (s, 1H), 7.60 (s, 1H), 4.19-4.16 (m, 1H), 3.65-3.59 (m, 2H), 3.31-314 (m, 2H), 2.96 (s, 3H), 2.79-2.66 (m, 2H), 2.36-2.34 (m, 2H), 1.57-1.55 (m, 1H), 1.16 (s, 3H), 0.89 (s, 3H).

I-45-b: MS(ES): m/z: 478.21 [M+H]⁺, ¹H-NMR (400 MHz, DMSO-d₆): δ 13.36 (s, 1H), 8.41 (s, 1H), 8.18 (s, 1H), 8.02 (s, 1H), 7.97 (s, 1H), 7.95 (s, 1H), 7.59 (s, 1H), 4.18-4.16 (m, 1H), 3.64-3.59 (m, 2H), 3.31-314 (m, 2H), 2.96 (s, 3H), 2.91-2.89 (m, 2H), 2.36-2.34 (m, 2H), 1.57-1.55 (m, 1H), 1.16 (s, 3H), 0.89 (s, 3H).

The following compounds were prepared following the procedure of 1-45:

| Cmpd. | | |
|---|---|---|
| No. | Structure and Name | Analytical Data |
| I-50 | 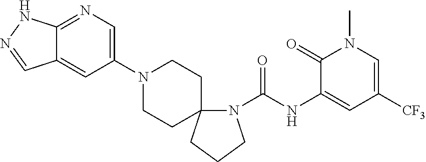 [SMILES: Cn1cc(C(F)(F)F)cc(NC(=O)N2CCCC23CCN(c2cnc4[nH]ncc4c2)CC3)c1=O] | MS (ES): m/z 476.19 [M + H]⁺ ¹H-NMR (400 MHz, DMSO-d6): δ 13.36 (s, 1H), 8.44 (s, 1H), 8.14 (s, 1H), 8.00 (s, 1H), 7.95 (s, 1H), 7.61 (s, 1H), 7.59 (s, 1H), 3.62 (s, 3H), 3.58-3.54 (m, 2H), 3.17- 3.16 (m, 1H), 3.01-2.96 (m, 2H), 2.79- 2.73 (m, 3H), 1.99-1.98 (m, 2H), 1.91- 1.86 (m, 2H), 1.46-1.43 (m, 2H) |
| | N-(1-methyl-2-oxo-5-(trifluoromethyl)- | |
| | 1,2-dihydropyridin-3-yl)-8-(1H- | |
| | pyrazolo[3,4-b]pyridin-5-yl)-1,8- | |
| | diazaspiro[4.5]decane-1-carboxamide | |
| I-61-aª and I- 61-b^{b} | 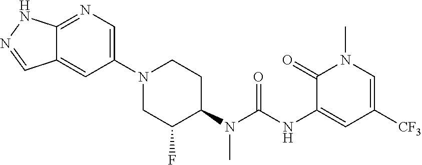 [SMILES: CN(C(=O)Nc1cc(C(F)(F)F)cn(C)c1=O)[C@@H]1CCN(c2cnc3[nH]ncc3c2)C[C@H]1F] | MS (ES): m/z 468.40 [M + H]⁺ ¹H NMR (DMSO-d6, 400 MHz): δ 13.41 (s, 1H), 8.49 (s, 1H), 8.15 (s, 1H), 8.05 (s, 1H), 7.98 (s, 2H), 7.72 (s, 1H), 4.98-4.97 (m, 1H), 4.86-4.85 (m, 1H), 4.37-4.27 (m, 1H), 4.05-3.97 (m, 1H), 3.61 (s, 3H), 2.99 (s, 3H), 2.76-2.87 (m, 2H), 2.06-0.97 (m, 1H), 1.83-1.76 (m, 1H) |
| | 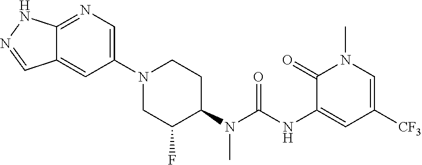 [SMILES: CN(C(=O)Nc1cc(C(F)(F)F)cn(C)c1=O)[C@@H]1CCN(c2cnc3[nH]ncc3c2)C[C@H]1F] | |
| | 1-((3S,4S)-3-fluoro-1-(1H-pyrazolo[3,4- | |
| | b]pyridin-5-yl)piperidin-4-yl)-1-methyl- | |
| | 3-(1-methyl-2-oxo-5-(trifluoromethyl)- | |
| | 1,2-dihydropyridin-3-yl)urea; | |
| | 1-((3R,4R)-3-fluoro-1-(1H-pyrazolo[3,4- | |
| | b]pyridin-5-yl)piperidin-4-yl)-1-methyl- | |
| | 3-(1-methyl-2-oxo-5-(trifluoromethyl)- | |
| | 1,2-dihydropyridin-3-yl)urea | |
| I-62-aª and I- 62-b^{b} | 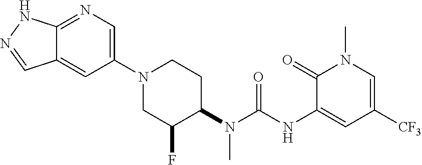 [SMILES: CN(C(=O)Nc1cc(C(F)(F)F)cn(C)c1=O)[C@@H]1CCN(c2cnc3[nH]ncc3c2)C[C@@H]1F] | MS (ES): m/z 468.41 [M + H]⁺ ¹H NMR (DMSO-d₆, 400 MHz): δ 13.37 (s, 1H), 8.45-8.44 (d, J = 2.4 Hz, 1H), 8.18-8.18 (d, J = 1.6 Hz, 1H), 8.05 (s, 1H), 7.99 (s, 1H), 7.98-7.97 (d, J = 8.0 Hz, 1H), 7.61-7.60 (d, J = 2.4 Hz, 1H), 4.99- 4.86 (d, J_{HF} = 50.8 Hz, 1H), 4.47-4.35 (m, 1H), 3.88-3.75 (m, 2H), 3.61 (s, 3H), 3.17- 3.05 (m, 1H), 3.02 (s, 3H), 2.98-2.92 (m, 1H), 2.34-1.32 (m, 1H), 1.69-1.65 (m, 1H) |
| | 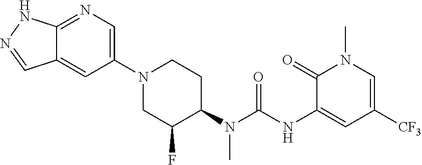 [SMILES: CN(C(=O)Nc1cc(C(F)(F)F)cn(C)c1=O)[C@@H]1CCN(c2cnc3[nH]ncc3c2)C[C@@H]1F] | |
| | 1-((3R,4S)-3-fluoro-1-(1H-pyrazolo[3,4- | |
| | b]pyridin-5-yl)piperidin-4-yl)-1-methyl- | |
| | 3-(1-methyl-2-oxo-5-(trifluoromethyl)- | |
| | 1,2-dihydropyridin-3-yl)urea; | |
| | 1-((3S,4R)-3-fluoro-1-(1H-pyrazolo[3,4- | |
| | b]pyridin-5-yl)piperidin-4-yl)-1-methyl- | |
| | 3-(1-methyl-2-oxo-5-(trifluoromethyl)- | |
| | 1,2-dihydropyridin-3-yl)urea | |
| I-64-a^{c} and I- 64-b^{d} | 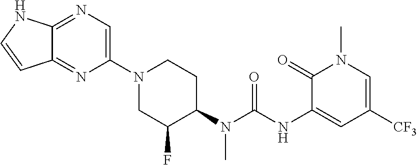 [SMILES: CN(C(=O)Nc1cc(C(F)(F)F)cn(C)c1=O)[C@@H]1CCN(c2cnc3[nH]ccc3n2)C[C@@H]1F] | MS (ES): m/z 468.45 [M + H]⁺ ¹H NMR (DMSO-d6, 400 MHz): δ 11.55 (s, 1H), 8.19-8.18 (d, J = 2.4 Hz, 1H), 8.06-8.05 (m, 2H), 7.97 (s, 1H), 7.58- 7.57 (t, J = 3.2 Hz, 1H), 6.32-6.31 (dd, J = 3.2 Hz, J = 2.0 Hz, 1H), 4.93 (d, J_{HF} = 50.4 Hz, 1H), 4.59-4.56 (m, 1H), 4.49- 4.46 (m, 2H), 3.59 (s, 3H), 3.21-3.16 (m, 1H), 3.10-3.07 (m, 1H), 2.96 (s, 3H), 2.25-2.22 (m, 1H), 1.65-1.63 (m, 1H) |
| | 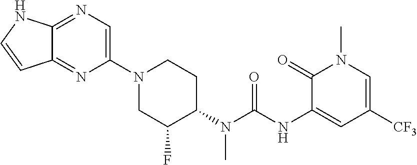 [SMILES: CN(C(=O)Nc1cc(C(F)(F)F)cn(C)c1=O)[C@H]1CCN(c2cnc3[nH]ccc3n2)C[C@H]1F] | |
| | 1-((3S,4R)-3-fluoro-1-(5H-pyrrolo[2,3- | |
| | b]pyrazin-2-yl)piperidin-4-yl)-1-methyl- | |
| | 3-(1-methyl-2-oxo-5-(trifluoromethyl)- | |
| | 1,2-dihydropyridin-3-yl)urea; | |
| | 1-((3R,4S)-3-fluoro-1-(5HI-pyrrolo[2,3- | |
| | b]pyrazin-2-yl)piperidin-4-yl)-1-methyl- | |
| | 3-(1-methyl-2-oxo-5-(trifluoromethyl)- | |
| | 1,2-dihydropyridin-3-yl)urea | |
| I-83-a^{e} and I- 83-b^{f} | 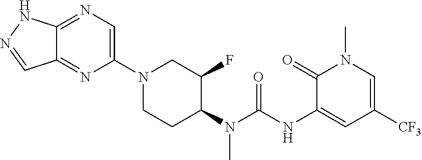 [SMILES: CN(C(=O)Nc1cc(C(F)(F)F)cn(C)c1=O)[C@H]1CCN(c2cnc3[nH]ncc3n2)C[C@H]1F] | MS (ES): m/z 469.33 [M + H]⁺ ¹H-NMR (400 MHz, DMSO-d6): δ 13.60 (s, 1H), 8.56 (s, 1H), 8.13 (s, 1H), 8.04 (s, 1H), 8.03 (s, 1H), 7.96 (s, 1H), 4.85-4.76 (m, 2H), 4.42-4.39 (m, 2H), 3.58 (s, 3H), 3.04-3.02 (m, 2H), 2.92 (s, 3H), 1.87-1.79 (m, 2H) |
| | 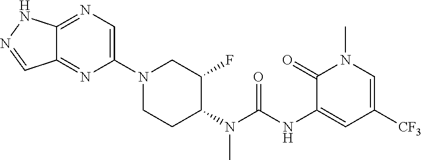 [SMILES: CN(C(=O)Nc1cc(C(F)(F)F)cn(C)c1=O)[C@@H]1CCN(c2cnc3[nH]ncc3n2)C[C@@H]1F] | |
| | 1-((3R,4S)-3-fluoro-1-(1H-pyrazolo[3,4- | |
| | b]pyrazin-5-yl)piperidin-4-yl)-1-methyl- | |
| | 3-(1-methyl-2-oxo-5-(trifluoromethyl)- | |
| | 1,2-dihydropyridin-3-yl)urea; | |
| | 1-((3S,4R)-3-fluoro-1-(1H-pyrazolo[3,4- | |
| | b]pyrazin-5-yl)piperidin-4-yl)-1-methyl- | |
| | 3-(1-methyl-2-oxo-5-(trifluoromethyl)- | |
| | 1,2-dihydropyridin-3-yl)urea | |

^{a}Separated by chiral HPLC (Method D), first eluting fraction;
^{b}Separated by chiral HPLC (Method D), second eluting fraction;
^{c}Separated by chiral HPLC (Method C), first eluting fraction;
^{d}Separated by chiral HPLC (Method C), second eluting fraction;
^{e}Separated by chiral SFC (Method E), first eluting fraction;
^{f}Separated by chiral SFC (Method E), second eluting fraction.

### Example 46: 1-(1-(3-(dimethylamino)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

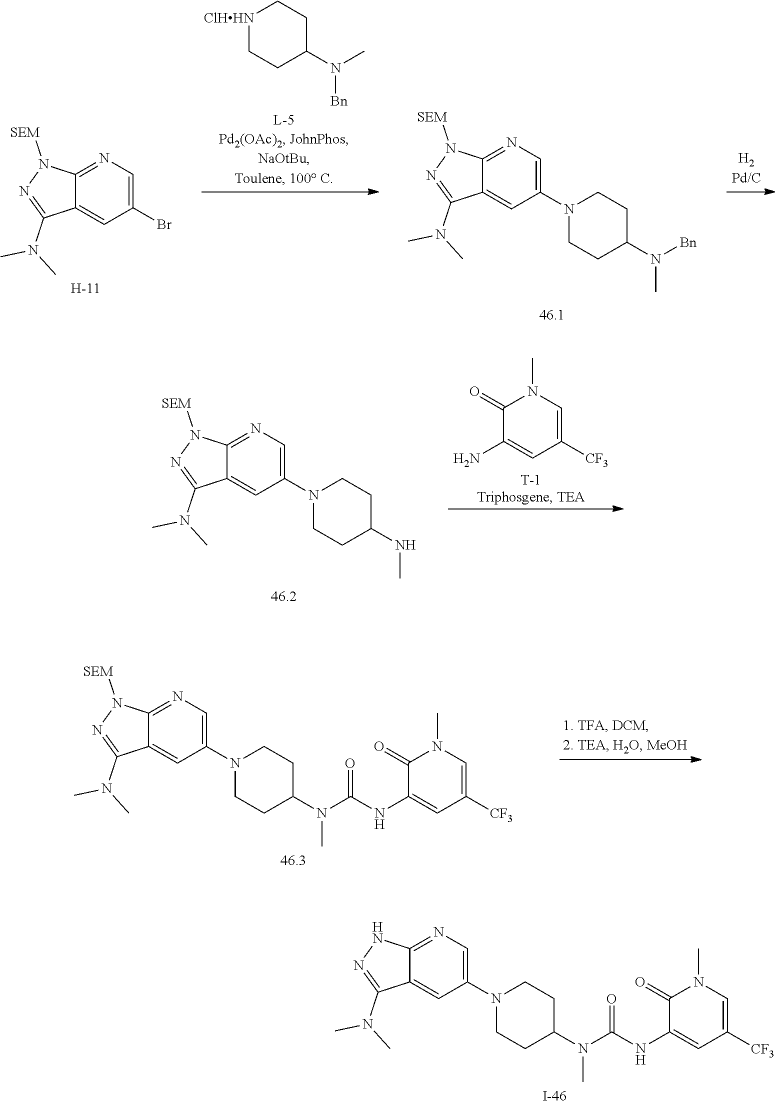

Synthesis of compound 46.1. A mixture of H-11 (0.45 g, 1.21 mmol, 1.0 equiv), L-5 (0.322 g, 1.58 mmol, 1.3 equiv) and sodium tert-butoxide (0.350 g, 3.64 mmol, 3.0 equiv) in toluene (5.0 mL) was purged with argon for 15 min. To the mixture was added (2-biphenyl)di-tert-butylphosphine (0.072 g, 0.24 mmol, 0.2 equiv) and palladium(II) acetate (0.081 g, 0.364 mmol, 0.1 equiv). The reaction mixture was stirred at 100° C. for 6 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford 46.1 (0.250 g, 42%). MS(ES): m/z 495.32 [M+H]⁺.

Synthesis of compound 46.2. A mixture of compound 46.1 (0.25 g, 0.50 mmol, 1.0 equiv) and 10% palladium hydroxide on carbon (0.3 g) in methanol (2.0 mL) was added under hydrogen (1 atm) for 2 h. The reaction mixture was filtered through a pad of Celite® and rinsed with methanol. The filtrate was concentrated under reduced pressure to afford 46.2 (0.15 g, 74%). MS(ES): m/z 405.27 [M+H]⁺.

Synthesis of compound 46.3. Compound 46.3 was prepared from compound 46.2 and T-1 following the procedure described in the synthesis of I-9. The product was purified by flash column chromatography on silica gel (CombiFlash®, 2% methanol in DCM). MS(ES): m/z 623.30 [M+H]⁺.

Synthesis of I-46. Compound I-46 was prepared from compound 46.3 following the procedure described in the synthesis of I-11. The product was purified by flash column chromatography on silica gel (CombiFlash®, 3.0% methanol in DCM). MS(ES): m/z 493.44 [M+H]⁺, ¹H NMR (DMSO-d₆, 400 MHz): δ 11.36 (s, 1H), 8.31 (s, 1H), 8.15 (s, 1H), 7.99 (s, 1H), 7.89 (s, 1H), 7.63 (s, 1H), 4.16-4.12 (i, 1H), 3.63-3.59 (m, 1H), 3.58 (s, 3H), 2.96 (s, 6H), 2.90 (s, 3H), 2.80-2.74 (9, 2H), 1.92-1.90 (m, 3H), 1.69-1.67 (1, 2H).

The following compounds were prepared following the procedure of I-46:

| Cmpd. | | |
|---|---|---|
| No. | Structure and Name | Analytical Data |
| I-49-aª | 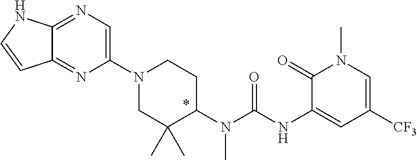 [SMILES: CN(C(=O)Nc1cc(C(F)(F)F)cn(C)c1=O)C1CCN(c2cnc3[nH]ccc3n2)CC1(C)C] | MS (ES): m/z 478.36 [M + H]⁺ ¹H-NMR (400 MHz, DMSO-d₆): δ 13.36 (s, 1H), 8.44 (s, 1H), 8.14 (s, 1H), 8.00 (s, 1H), 7.95 (s, 1H), 7.61 (s, 1H), 7.59 (s, 1H), 3.62 (s, 3H), 3.58-3.54 (m, 2H), 3.17-3.16 (m, 1H), 3.01-2.96 (m, 2H), 2.79-2.73 (m, 3H), 1.99-1.98 (m, 2H), 1.91-1.86 (m, 2H), 1.46-1.43 (m, 2H) |
| | (R)- or (S)-1-(3,3-dimethyl-1-(5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-49-b^{b} | 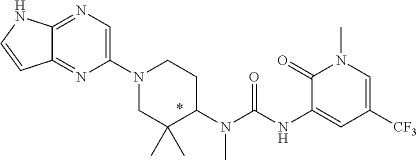 [SMILES: CN(C(=O)Nc1cc(C(F)(F)F)cn(C)c1=O)C1CCN(c2cnc3[nH]ccc3n2)CC1(C)C] | MS (ES): m/z 478.41 [M + H]⁺ ¹H-NMR (400 MHz, DMSO-d₆): δ 11.51 (s, 1H), 8.18 (s, 1H), 8.05-8.02 (d, J = 8.0 Hz, 2H), 7.96 (s, 1H), 7.55 (s, 1H), 6.30 (s, 1H), 4.45-4.41 (m, 1H), 4.26- 4.23 (m, 1H), 3.92-3.91 (1H), 3.59 (s,3H), 2.93 (s, 3H), 2.19-2.17 (m, 1H), 1.54-1.52 (m, 1H), 1.32-1.13 (m, 2H), 0.99-0.92 (m, 6H) |
| | (S)- or (R)-1-(3,3-dimethyl-1-(5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-63-a^{c} and I- 63-b^{d} | 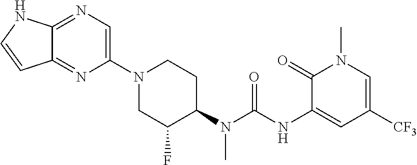 [SMILES: CN(C(=O)Nc1cc(C(F)(F)F)cn(C)c1=O)[C@@H]1CCN(c2cnc3[nH]ccc3n2)C[C@H]1F] | MS (ES): m/z 468.36 [M + H]⁺ ¹H NMR (DMSO-d₆, 400 MHz): δ 11.55 (s, 1H), 8.19-8.18 (d, J = 2.4 Hz, 1H), 8.06-8.05 (m, 2H), 7.97 (s, 1H), 7.58- 7.57 (t, J = 3.2 Hz, 1H), 6.32-6.31 (dd, J = 3.2 Hz, J = 2.0 Hz, 1H), 4.93 (d, J_{HF} = 50.4 Hz, 1H), 4.59-4.56 (m, 1H), 4.49- 4.46 (m, 2H), 3.59 (s, 3H), 3.21-3.16 (m, 1H), 3.10-3.07 (m, 1H), 2.96 (s, 3H), 2.25-2.22 (m, 1H), 1.65-1.63 (m, 1H) |
| | 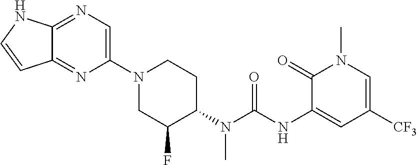 [SMILES: CN(C(=O)Nc1cc(C(F)(F)F)cn(C)c1=O)[C@H]1CCN(c2cnc3[nH]ccc3n2)C[C@@H]1F] | |
| | 1-((3R,4R)-3-fluoro-1-(5H-pyrrolo[2,3- | |
| | b]pyrazin-2-yl)piperidin-4-yl)-1-methyl- | |
| | 3-(1-methyl-2-oxo-5-(trifluoromethyl)- | |
| | 1,2-dihydropyridin-3-yl)urea | |
| | 1-((35,4S)-3-fluoro-1-(5H-pyrrolo[2,3- | |
| | b]pyrazin-2-yl)piperidin-4-yl)-1-methyl- | |
| | 3-(1-methyl-2-oxo-5-(trifluoromethyl)- | |
| | 1,2-dihydropyridin-3-yl)urea | |

^{a}Separated by chiral SFC (Method H); first eluting isomer;
^{b}Separated by chiral SFC (Method H); second eluting isomer;
^{c}Separated by chiral SFC (Method B); first eluting isomer;
^{d}Separated by chiral SFC (Method B); second eluting isomer.
*Absolute stereochemistry not determined.

### Example 48: 1,1-dimethyl-3-(5-(4-(1-methyl-3-(1-methyl-2-oxo-5-(trifluoromethyl)-1,2-dihydropyridin-3-yl)ureido)piperidin-1-yl)pyrazin-2-yl)urea

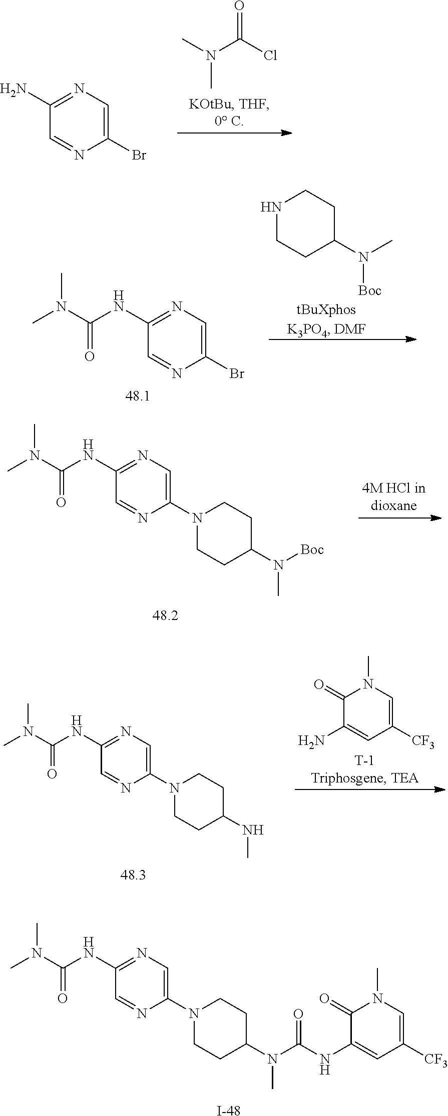

Synthesis of compound 48.1. To a solution of 5-bromopyrazin-2-amine (1.0 g, 5.74 mmol, 1.0 equiv) in THF (10 mL) was added dimethylcarbamic chloride (0.680 g, 6.32 mmol, 1.1 equiv) at 0° C. To the solution was added potassium tert-butoxide (1 M in THF, 8.6 mL, 11.49 mmol, 2.0 equiv). The reaction mixture was stirred at room temperature for 1 h. It transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford 48.1 (1.0 g, 71%). m/z: 246.23 [M+H]⁺.

Synthesis of compound 48.2. To a solution of 48.1 (0.75 g, 3.06 mmol, 1.0 equiv) in DMF (10 mL) was added tert-butyl methyl(piperidin-4-yl)carbamate (0.982 g, 4.59 mmol, 1.5 equiv), potassium phosphate (1.94 g, 9.18 mmol, 3.0 equiv) and purged with argon for 10 min. tBuXPhos-Pd-G3 was added (0.364 g, 0.738 mmol, 0.15 equiv) and the reaction mixture was stirred at room temperature overnight. It was filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (1.5% methanol in DCM) to afford 48.2 (0.27 g, 23.31%). MS(ES): m/z 379.24 [M+H]⁺.

Synthesis of compound 48.3. To a solution of 48.2 (0.27 g, 0.714 mmol, 1.0 equiv) in DCM (2 mL) was added 4 M hydrochloric acid in dioxane (2 mL) dropwise at 0° C. The reaction mixture was stirred at room temperature for 1 h. It was transferred into a saturated aqueous solution of sodium bicarbonate and extracted with DCM. The combined organic layers were washed with brine, dried over anhydrous sodium sulfate, filtered, and concentrated under reduced pressure to afford 48.3 (0.17 g, 85%). MS(ES): m/z 279.19 [M+H]⁺.

Synthesis of compound I-48. Compound I-48 was prepared from compound 48.2 and T-1 following the procedure described in the synthesis of I-9. The product was purified by flash column chromatography on silica gel (1.5% methanol in DCM). MS(ES): m/z 497.22 [M+H]⁺, ¹H NMR (DMSO-d₆, 400 MHz): δ 8.63 (s, 1H), 8.47 (s, 1H), 8.17 (s, 1H), 8.04 (s, 1H), 8.02 (s, 1H), 7.89 (s, 1H), 4.35-4.32 (m, 3H), 3.58 (s, 3H), 2.92 (s, 6H), 2.88-2.87 (m, 2H), 2.83 (s, 3H), 1.70-1.66 (m, 4H).

### Example 51: 1-methyl-1-(4-methyl-1-(5H-pyrrolo[2,3-b]pyrazin-2-yl)piperidin-4-yl)-3-(1-methyl-2-oxo-5-(trifluoromethyl)-1,2-dihydropyridin-3-yl)urea

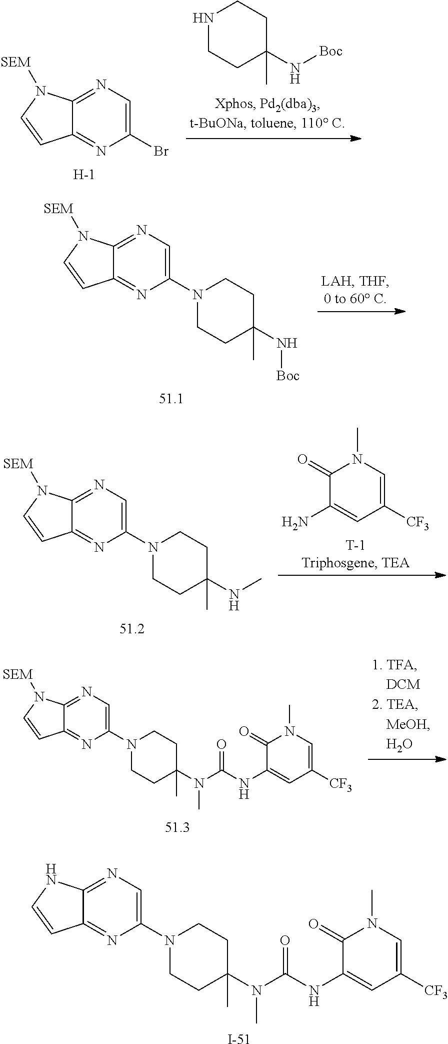

Synthesis of compound 51.1. A mixture of H-1 (2.0 g, 6.06 mmol, 1.0 equiv), tert-butyl (4-methylpiperidin-4-yl)carbamate (1.3 g, 6.06 mmol, 1.0 equiv) and sodium tert-butoxide (1.7 g, 18.18 mmol, 3.0 equiv) in toluene (10 mL) was degassed by bubbling through a stream of argon for 10 min. 2-Dicyclohexylphosphino-2′,4′,6′-triisopropylbiphenyl (0.578 g, 1.21 mmol, 0.2 equiv) and tris(dibenzylideneacetone)dipalladium (0.555 g, 0.606 mmol, 0.1 equiv) were added and degassed for 5 min. The mixture was stirred in a microwave reactor at 110° C. for 1 h. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.5% methanol in DCM) to afford 51.1 (1.2 g, 43%). MS(ES): m/z 462.28 [M+H]⁺.

Synthesis of compound 51.2. To a solution of 51.1 (1.0 g, 2.25 mmol, 1.0 equiv) in THF (10 mL) at 0° C. was added lithium aluminum hydride (1 M in THF, 2.48 mL, 2.48 mmol, 1.1 equiv). The mixture was stirred at 60° C. for 15 min. It was quenched with saturated solution of ammonium chloride. The solid formed was filtered through sintered and filtrate was extracted with ethyl acetate. The combined organic layers were washed with brine, dried over anhydrous sodium sulfate, filtered, and concentrated under reduced pressure. The residue was purified by reversed phase column chromatography (acetonitrile in water) to afford 51.2 (0.40 g, 42%). MS(ES): m/z 376.25 [M+H]⁺.

Synthesis of compound 51.3. Compound 51.3 was prepared from compound 51.2 and T-1 following the procedure described in the synthesis of I-9. The product was purified by flash column chromatography on silica gel (3.2% methanol in DCM). MS(ES): m/z 594.27 [M+H]⁺.

Synthesis of compound I-51. Compound I-51 was prepared from compound 51.3 and following the procedure described in the synthesis of I-11. The product was purified by flash column chromatography on silica gel (CombiFlash®, 2.5% methanol in DCM). MS(ES): m/z 464.59 [M+H]⁺, ¹H-NMR (400 MHz, DMSO-d₆): δ 11.50 (s, 1H), 8.08-8.07 (m, 2H), 8.01 (s, 1H), 7.99 (s, 1H), 7.56-7.55 (m, 1H), 6.31-6.30 (m, 1H), 3.74-3.71 (m, 2H), 3.56 (s, 3H), 3.23-3.18 (m, 2H), 2.97 (s, 3H), 2.35-2.37 (m, 2H), 1.76-1.71 (m, 2H), 1.33 (s, 3H).

The following compound was prepared following the procedure of I-53:

| Cmpd. | | |
|---|---|---|
| No. | Structure and Name | Analytical Data |
| I-56 | 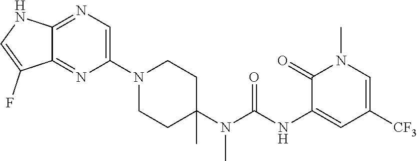 [SMILES: CN(C(=O)Nc1cc(C(F)(F)F)cn(C)c1=O)C1(C)CCN(c2cnc3[nH]cc(F)c3n2)CC1] | MS (ES): m/z 482.33 [M + H]⁺ ¹H-NMR (400 MHz, DMSO-d₆): δ 11.36 (s, 1H), 8.12 (s, 1H), 8.07-8.06 (m, 2H), 7.98 (s, 1H), 7.55 (s, 1H), 3.78-3.75 (m, 2H), 3.55 (s, 3H), 3.23-3.17 (m, 4H), 2.96 (s, 3H), 1.74-1.68 (m, 2H), 1.32 (s, 3H) |
| | 1-(1-(7-fluoro-5H-pyrrolo[2,3-b]pyrazin-2- | |
| | yl)-4-methylpiperidin-4-yl)-1-methyl-3-(1- | |
| | methyl-2-oxo-5-(trifluoromethyl)-1,2- | |
| | dihydropyridin-3-yl)urea | |

### Example 53: (S)-3-methyl-N-(1-methyl-2-oxo-5-(trifluoromethyl)-1,2-dihydropyridin-3-yl)-8-(5H-pyrrolo[2,3-b]pyrazin-2-yl)-1,8-diazaspiro[4.5]decane-1-carboxamide and (R)-3-methyl-N-(1-methyl-2-oxo-5-(trifluoromethyl)-1,2-dihydropyridin-3-yl)-8-(5H-pyrrolo[2,3-b]pyrazin-2-yl)-1,8-diazaspiro[4.5]decane-1-carboxamide

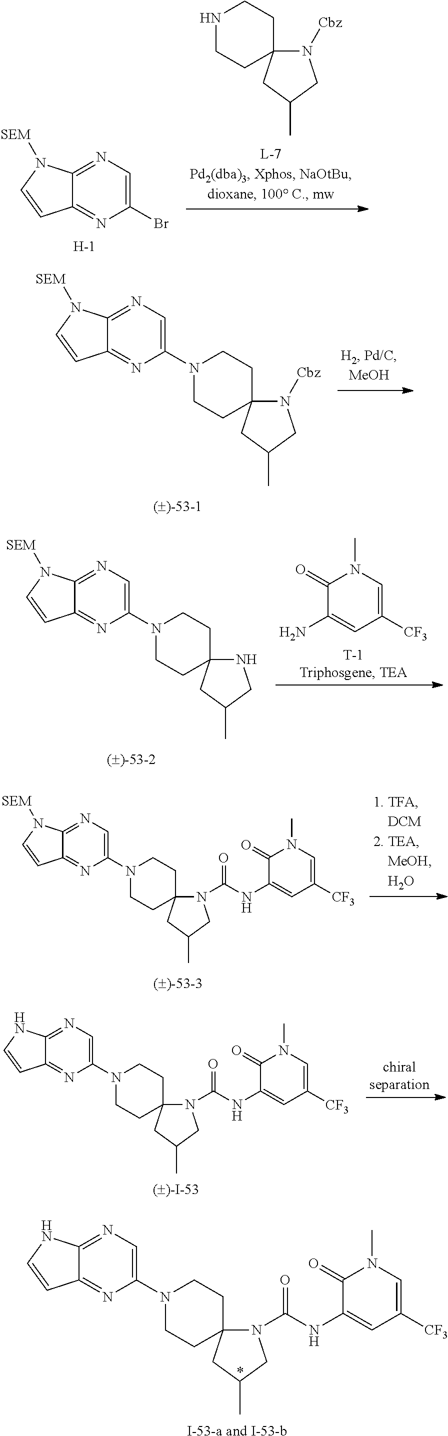

Synthesis of compound (±)-53.1. Compound (±)-53.1 was prepared from compound H-1 and L-7 following the procedure described in the synthesis of 11.1. The product was purified by flash column chromatography on silica gel (CombiFlash®, 2.5% methanol in DCM). MS(ES): m/z: 536.30 [M+H]⁺.

Synthesis of compound (±)-53.2. A mixture of compound (±)-53.1 (0.25 g, 0.46 mmol, 1.0 equiv) and 10% palladium on carbon (0.05 g) in methanol (2.0 mL) was stirred under hydrogen (1 atm) for 3.0 h. The reaction mixture was filtered through a pad of Celite® and rinsed with methanol. The filtrate was concentrated under reduced pressure to afford (±)-53.2 (0.18 g, 96%). MS(ES): m/z 402.26 [M+H]⁺.

Synthesis of compound (±)-53.3. Compound (±)-53.3 was prepared from compound (±)-53.2 and T-1 following the procedure described in the synthesis of I-9. The product was purified by flash column chromatography on silica gel (3.0% methanol in DCM). MS(ES): m/z 620.29 [M+H]⁺.

Synthesis of compound (±)-I-53. Compound (±)-I-53 was prepared from compound (±)-53.3 following the procedure described in the synthesis of I-11. The product was purified by flash column chromatography on silica gel (CombiFlash®, 3.1% methanol in DCM). MS(ES): m/z 490.21 [M+H]⁺.

I-53-a and I-53-b: The racemate was separated by chiral SFC (Method D) to afford first eluting fraction (I-53-a) and second eluting fraction (I-53-b). (*Absolute configuration was not determined.)

I-53-a: MS(ES): m/z: 490.21 [M+H]⁺, ¹H-NMR (400 MHz, DMSO-d₆): δ 11.53 (s, 1H), 8.04 (s, 1H), 8.02 (s, 1H), 8.01 (s, 1H), 7.98 (s, 1H), 7.56 (s, 1H), 6.30 (s, 1H), 4.26-4.24 (m, 2H), 3.67-3.65 (m, 1H), 3.57 (s, 3H), 3.09-3.07 (m, 1H), 3.02-2.92 (m, 2H), 2.83-2.72 (m, 1H), 2.35-2.33 (m, 1H), 1.51-1.47 (m, 1H), 1.42-1.33 (m, 2H), 1.27-1.21 (m, 1H), 1.09-1.07 (m, 1H), 1.04-1.01 (m, 3H).

I-53-b. MS(ES): m/z: 490.25 [M+H]⁺, ¹H-NMR (400 MHz, DMSO-d₆): δ 11.53 (s, 1H), 8.04-8.04 (d, J=2.4 Hz, 1H), 8.02 (s, 1H), 7.98 (s, 1H), 7.56-7.55 (d, J=3.6 Hz, 2H), 6.30-6.29 (m, 1H), 4.26-4.24 (m, 2H), 3.66-3.64 (m, 1H), 3.56 (s, 3H), 3.11-3.07 (m, 1H), 3.06-2.85 (m, 2H), 2.85-2.76 (m, 1H), 2.69-2.53 (m, 1H), 2.41-2.32 (m, 1H), 1.51-1.47 (m, 1H), 1.41-1.38 (m, 2H), 1.33-1.32 (m, 1H), 1.08-1.07 (d, J=6.4 Hz, 3H).

The following compounds were prepared following the procedure of I-53:

| Cmpd. | | |
|---|---|---|
| No. | Structure and Name | Analytical Data |
| I-54-aª and I- 54-b^{b} | 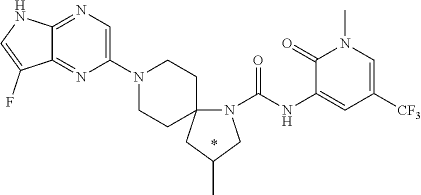 [SMILES: CC1CN(C(=O)Nc2cc(C(F)(F)F)cn(C)c2=O)C2(CCN(c3cnc4[nH]cc(F)c4n3)CC2)C1] | MS (ES) m/z 508.48 [M + H]⁺ ¹H-NMR (400 MHz, DMSO-d₆): δ 11.40 (s, 1H), 8.14 (s, 1H), 8.04-8.03 (d, J = 2.4 Hz, 1H), 7.97 (s, 1H), 7.56 (s, 2H), 4.37-4.29 (m, 2H), 3.69-3.65 (m, 1H), 3.56 (s, 3H), 3.12-3.07 (m, 1H), 3.01- 2.93 (m, 2H), 2.86-2.83 (m, 1H), 2.35- 2.33 (m, 1H), 1.52-1.49 (m, 1H), 1.43- 1.33 (m, 3H), 1.24-1.21 (m, 1H), 1.09- 1.07 (d, J = 6.4 Hz, 3H). |
| | (R)-or (S)-8-(7-fluoro-5H-pyrrolo[2,3- | |
| | b]pyrazin-2-yl)-3-methyl-N-(1-methyl-2- | |
| | oxo-5-(trifluoromethyl)-1,2- | |
| | dihydropyridin-3-yl)-1,8- | |
| | diazaspiro[4.5]decane-1-carboxamide | |
| I-55 | 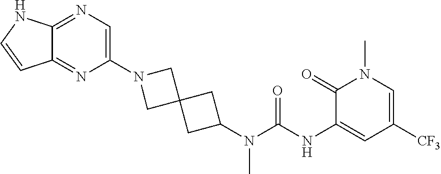 [SMILES: CN(C(=O)Nc1cc(C(F)(F)F)cn(C)c1=O)C1CC2(C1)CN(c1cnc3[nH]ccc3n1)C2] | MS (ES) m/z 462.10 [M + H]⁺ ¹H-NMR (400 MHz, DMSO-d₆): δ 11.54 (s, 1H), 8.14 (s, 1H), 8.02 (s, 1H), 7.85 (s, 1H), 7.56-7.55 (d, J = 4.0 Hz, 1H), 7.54-7.53 (d, J = 3.2 Hz, 1H), 6.30 (s, 1H), 4.46-4.45 (m, 1H), 4.06 (s, 2H), 3.93 (s, 2H), 3.58 (s, 3H), 2.90 (s, 3H), 2.43-2.32 (m, 4H) |
| | 1-(2-(5H-pyrrolo[2,3-b]pyrazin-2-yl)-2- | |
| | azaspiro[3.3]heptan-6-yl)-1-methyl-3-(1- | |
| | methyl-2-oxo-5-(trifluoromethyl)-1,2- | |
| | dihydropyridin-3-yl)urea | |
| I-57 | 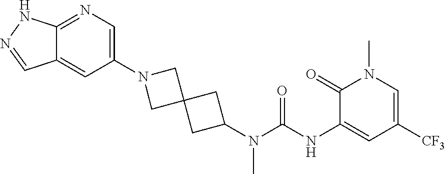 [SMILES: CN(C(=O)Nc1cc(C(F)(F)F)cn(C)c1=O)C1CC2(C1)CN(c1cnc3[nH]ncc3c1)C2] | MS (ES) m/z 462.42 [M + H]⁺ ¹H-NMR (400 MHz, DMSO-d₆): δ 13.28 (s, 1H), 8.14 (s, 1H), 8.02 (s, 1H), 7.94- 7.93 (d, J = 2.4 Hz, 1H), 7.90 (s, 1H), 7.85 (s, 1H), 7.10 (s, 1H), 4.47-4.43 (m, 1H), 3.94 (s, 2H), 3.86 (s, 2H), 3.58 (s, 3H), 2.90 (s, 3H), 2.43-2.38 (m, 2H), 2.33-2.32 (m, 2H) |
| | 1-(2-(1H-pyrazolo[3,4-b]pyridin-5-yl)-2- | |
| | azaspiro[3.3 ]heptan-6-yl)-1-methyl-3-(1- | |
| | methyl-2-oxo-5-(trifluoromethyl)-1,2- | |
| | dihydropyridin-3-yl)urea | |
| I-67 | 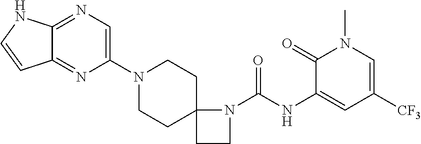 [SMILES: Cn1cc(C(F)(F)F)cc(NC(=O)N2CCC23CCN(c2cnc4[nH]ccc4n2)CC3)c1=O] | MS (ES) m/z 462.41 [M + H]⁺ ¹H NMR (DMSO-d₆, 400 MHz): δ 11.50 (s, 1H), 8.51 (s, 1H), 8.20 (s, 1H), 7.99 (s, 1H), 7.92 (s, 1H), 7.55-7.54 (t, J = 2.8 Hz, 1H), 6.30-6.29 (m, 1H), 4.27-4.19 (m, 2H), 3.56 (s, 3H), 3.19-3.12 (m, 2H), 2.81-2.73 (m, 2H), 1.76-1.1.73 (m, 2H), 1.59-1.51 (m, 1H), 1.41-1.37 (m, 1H), 1.17-1.21 (m, 2H) |
| | N-(1-methyl-2-oxo-5-(trifluoromethyl)- | |
| | 1,2-dihydropyridin-3-yl)-7-(5H- | |
| | pyrrolo[2,3-b]pyrazin-2-yl)-1,7- | |
| | diazaspiro[3.5]nonane-1-carboxamide | |
| I-72 | 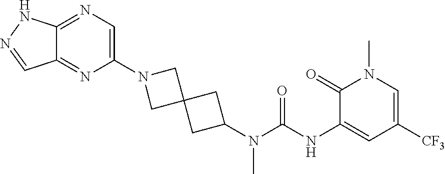 [SMILES: CN(C(=O)Nc1cc(C(F)(F)F)cn(C)c1=O)C1CC2(C1)CN(c1cnc3[nH]ncc3n1)C2] | MS (ES) m/z 463.27 [M + H]⁺ ¹H NMR (DMSO-d₆, 400 MHz): δ 13.55 (s, 1H), 8.14-8.13 (d, J = 2.0 Hz, 1H), 8.02 (s, 1H), 7.97 (s, 1H), 7.94 (s, 1H), 7.85 (s, 1H), 4.48-4.44 (m, 1H), 4.16 (s, 2H), 4.03 (s, 2H), 3.58 (s, 3H), 2.90 (s, 3H), 2.44-2.32 (m, 4H). |
| | 1-(2-(1H-pyrazolo[3,4-b]pyrazin-5-yl)-2- | |
| | azaspiro[3.3]heptan-6-yl)-1-methyl-3-(1- | |
| | methyl-2-oxo-5-(trifluoromethyl)-1,2- | |
| | dihydropyridin-3-yl)urea | |
| I-86 | 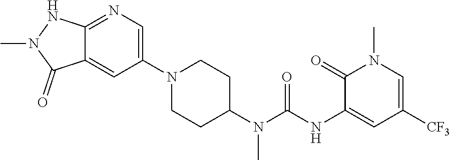 [SMILES: CN(C(=O)Nc1cc(C(F)(F)F)cn(C)c1=O)C1CCN(c2cnc3[nH]n(C)c(=O)c3c2)CC1] | MS (ES) m/z 480.2 [M + H]⁺ ¹H NMR (DMSO-d₆, 400 MHz): δ 11.47 (s, 1H), 8.44 (s 1H), 8.17 (s, 1H), 8.03 (s, 1H), 7.90 (s, 1H), 7.56 (s, 1H), 4.13-4.21 (m, 1H), 3.72-3.69 (d, J = 11.2 Hz, 2H), 3.58 (s, 3H), 3.35 (s, 3H), 288 (s, 3H), 2.80-2.75 (m, 2H), 1.91-1.83 (m, 2H), 1.67-1.65 (m, 2H) |
| | 1-methyl-3-(1-methyl-2-oxo-5- | |
| | (trifluoromethyl)-1,2-dihydropyridin-3- | |
| | yl)-1-(1-(2-methyl-3-oxo-2,3-dihydro-1H- | |
| | pyrazolo[3,4-b]pyridin-5-yl)piperidin-4- | |
| | yl)urea | |
| I-88-aª and I- 88-b^{b} | 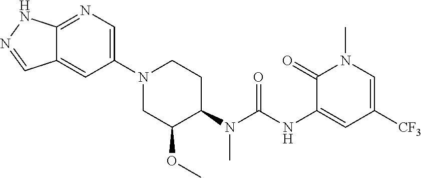 [SMILES: CO[C@H]1CN(c2cnc3[nH]ncc3c2)CC[C@H]1N(C)C(=O)Nc1cc(C(F)(F)F)cn(C)c1=O] | MS (ES) m/z 480.37 [M + H]⁺ ¹H-NMR (400 MHz, DMSO-d₆): δ 13.36 (s, 1H), 8.41 (s, 1H), 8.18 (s, 1H), 8.15 (s, 1H), 8.03 (s, 1H), 7.96 (s, 1H), 7.62 (s, 1H), 4.23-4.12 (m, 1H), 3.92-3.89 (m, 1H), 3.72-3.63 (1H), 3.59 (s, 3H), 3.32 (s, 3H), 3.03 (s, 3H), 2.86-2.83 (m, 1H), 2.76-2.73 (m, 1H), 2.43-2.33 (m, 1H), 1.62-1.53 (m, 2H) |
| | 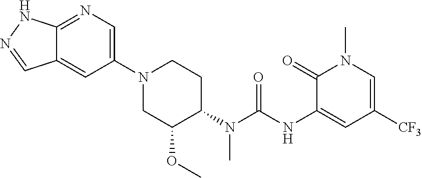 [SMILES: CO[C@@H]1CN(c2cnc3[nH]ncc3c2)CC[C@@H]1N(C)C(=O)Nc1cc(C(F)(F)F)cn(C)c1=O] | |
| | 1-((3S,4R)-3-methoxy-1-(1H- | |
| | pyrazolo[3,4-b]pyridin-5-yl)piperidin-4- | |
| | yl)-1-methyl-3-(1-methyl-2-oxo-5- | |
| | (trifluoromethyl)-1,2-dihydropyridin-3- | |
| | yl)urea | |
| | 1-((3R,4S)-3-methoxy-1-(1H- | |
| | pyrazolo[3,4-b]pyridin-5-yl)piperidin-4- | |
| | yl)-1-methyl-3-(1-methyl-2-oxo-5- | |
| | (trifluoromethyl)-1,2-dihydropyridin-3- | |
| | yl)urea | |
| I-89-a^{c} and I-89-b^{d} | 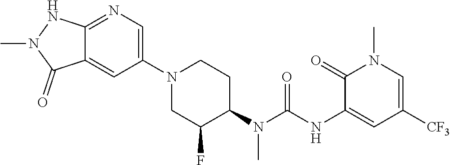 [SMILES: CN(C(=O)Nc1cc(C(F)(F)F)cn(C)c1=O)[C@@H]1CCN(c2cnc3[nH]n(C)c(=O)c3c2)C[C@@H]1F] | MS (ES) m/z 498.12 [M + H]⁺ ¹H NMR (DMSO-d₆, 400 MHz): δ 10.38 (s, 1H), 8.43 (s, 1H), 8.17 (s, 1H), 8.05 (s, 1H), 7.98 (s, 1H), 7.57 (s, 1H), 4.97- 4.85 (d, J_{HF} = 49.6 Hz, 1H), 4.46-4.35 (m, 1H), 4.11-4.09 (m, 2H), 3.89-3.82 (m, 1H), 3.59 (s, 3H), 3.32 (s, 3H), 3.21- 3.11 (m, 2H), 2.88 (s, 3H), 1.69-1.61 (m, 1H). |
| | 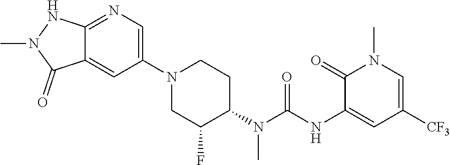 [SMILES: CN(C(=O)Nc1cc(C(F)(F)F)cn(C)c1=O)[C@H]1CCN(c2cnc3[nH]n(C)c(=O)c3c2)C[C@H]1F] | |
| | 1-((3S,4R)-3-fluoro-1-(2-methyl-3-oxo- | |
| | 2,3-dihydro-1H-pyrazolo[3,4-b]pyridin-5- | |
| | yl)piperidin-4-yl)-1-methyl-3-(1-methyl- | |
| | 2-oxo-5-(trifluoromethyl)-1,2- | |
| | dihydropyridin-3-yl)urea | |
| | 1-((3R,4S)-3-fluoro-1-(2-methyl-3-oxo- | |
| | 2,3-dihydro-1H-pyrazolo[3,4-b]pyridin-5- | |
| | yl)piperidin-4-yl)-1-methyl-3-(1-methyl- | |
| | 2-oxo-5-(trifluoromethyl)-1,2- | |
| | dihydropyridin-3-yl)urea | |
| 1-90-aª and I- 90-b^{b} | 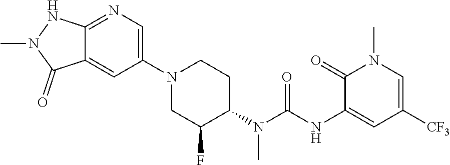 [SMILES: CN(C(=O)Nc1cc(C(F)(F)F)cn(C)c1=O)[C@H]1CCN(c2cnc3[nH]n(C)c(=O)c3c2)C[C@@H]1F] | MS (ES) m/z 498.29 [M + H]⁺ ¹H NMR (DMSO-d₆, 400 MHz): δ 10.31 (bs, 1H), 8.43 (s, 1H), 8.18 (s, 1H), 8.05 (s, 1H), 7.98 (s, 1H), 7.57 (s, 1H), 4.97- 4.85 (d, J_{HF} = 49.6 Hz, 1H), 4.46-4.35 (m, 1H), 3.92-3.87 (m, 3H), 3.59 (s, 3H), 3.32 (s, 2H), 3.17-3.15 (m, 2H), 3.00 (s, 3H), 2.32-2.29 (m, 1H), 1.66-1.63 (m, 1H) |
| | 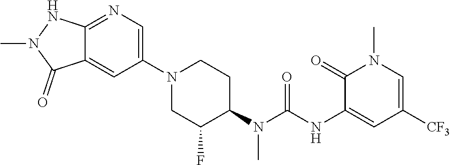 [SMILES: CN(C(=O)Nc1cc(C(F)(F)F)cn(C)c1=O)[C@@H]1CCN(c2cnc3[nH]n(C)c(=O)c3c2)C[C@H]1F] | |
| | 1-((3R,4S)-3-fluoro-1-(2-methyl-3-oxo- | |
| | 2,3-dihydro-1H-pyrazolo[3,4-b]pyridin-5- | |
| | yl)piperidin-4-yl)-1-methyl-3-(1-methyl- | |
| | 2-oxo-5-(trifluoromethyl)-1,2- | |
| | dihydropyridin-3-yl)urea | |
| | 1-((3R,4R)-3-fluoro-1-(2-methyl-3-oxo- | |
| | 2,3-dihydro-1H-pyrazolo[3,4-b]pyridin-5- | |
| | yl)piperidin-4-yl)-1-methyl-3-(1-methyl- | |
| | 2-oxo-5-(trifluoromethyl)-1,2- | |
| | dihydropyridin-3-yl)urea | |
| I-95-a^{c} and I-95-b^{d} | 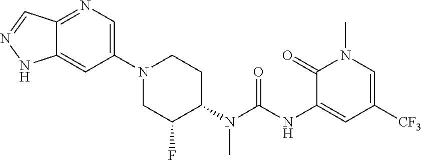 [SMILES: CN(C(=O)Nc1cc(C(F)(F)F)cn(C)c1=O)[C@H]1CCN(c2cnc3cn[nH]c3c2)C[C@H]1F] | MS (ES) m/z 468.7 [M + H]⁺ ¹H-NMR (400 MHz, DMSO-d₆): δ 12.88 (s, 1H), 8.44 (s, 1H), 8.18 (s, 1H), 8.06 (s, 1H), 8.05 (s, 1H), 7.99 (s, 1H), 7.22 (s, 1H), 5.02-4.89 (d, J_{HF} = 52 Hz, 1H), 4.52-4.08 (m, 1H), 4.12-3.97 (m, 2H), 3.60 (s, 3H), 3.21-3.18 (m, 1H), 3.08- 3.06 (m, 1H), 3.01 (s, 3H), 2.37-2.28 (m, 1H), 1.69-1.66 (m, 1H) |
| | 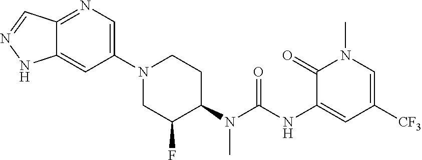 [SMILES: CN(C(=O)Nc1cc(C(F)(F)F)cn(C)c1=O)[C@@H]1CCN(c2cnc3cn[nH]c3c2)C[C@@H]1F] | |
| | 1-((3R,4S)-3-fluoro-1-(1H-pyrazolo[4,3- | |
| | b]pyridin-6-yl)piperidin-4-yl)-1-methyl-3- | |
| | (1-methyl-2-oxo-5-(trifluoromethyl)-1,2- | |
| | dihydropyridin-3-yl)urea | |
| | 1-((3S,4R)-3-fluoro-1-(1H-pyrazolo[4,3- | |
| | b]pyridin-6-yl)piperidin-4-yl)-1-methyl-3- | |
| | (1-methyl-2-oxo-5-(trifluoromethyl)-1,2- | |
| | dihydropyridin-3-yl)urea | |
| I-99 | 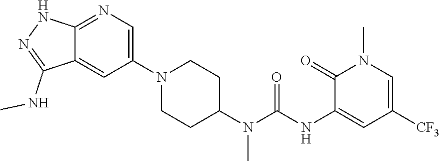 [SMILES: CNc1n[nH]c2ncc(N3CCC(N(C)C(=O)Nc4cc(C(F)(F)F)cn(C)c4=O)CC3)cc12] | MS (ES) m/z 479.14 [M + H]⁺ ¹H NMR (DMSO-d₆, 400 MHz): δ 11.69 (s, 1H), 8.28 (s, 1H), 8.17 (s, 1H), 8.03 (s, 1H), 7.91 (s, 1H), 7.64 (s, 1H), 5.93- 5.97 (m, 1H), 4.19-4.17 (m, 1H), 3.58 (s, 3H), 3.57-3.54 (m, 2H), 2.95 (s, 3H), 2.90 (s, 3H), 2.83-2.66 (m, 2H), 1.94- 1.90 (m, 2H), 1.70-1.67 (m, 2H) |
| | 1-methyl-3- | |
| | (1-methyl-2-oxo-5-(trifluoromethyl)-1,2- | |
| | dihydropyridin-3-yl)-1-(1-(3- | |
| | (methylamino)-1H-pyrazolo[3,4- | |
| | b]pyridin-5-yl)piperidin-4-yl)urea | |
| I-110- a^{e} and I-110- b^{f} | 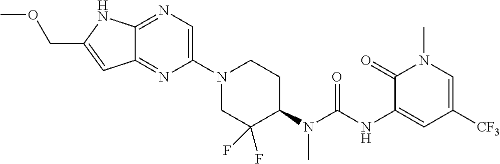 [SMILES: COCc1cc2nc(N3CC[C@@H](N(C)C(=O)Nc4cc(C(F)(F)F)cn(C)c4=O)C(F)(F)C3)cnc2[nH]1] | MS (ES) m/z 530.36 [M + H]⁺ ¹H-NMR (400 MHz, DMSO-d₆): δ 11.74 (s, 1H), 8.16 (s, 1H), 8.12 (s, 1H), 8.08 (s, 1H), 8.06 (s, 1H), 6.32 (s, 1H), 4.93- 4.84 (m, 1H), 4.68-4.62 (m, 1H), 4.53 (s, 1H), 4.46-4.43 (m, 1H), 3.60 (s, 3H), 3.29 (s, 3H), 3.13-3.10 (m, 1H), 2.98 (s, 3H), 2.21-2.19 (m, 1H), 1.88-1.84 (m, 1H) |
| | 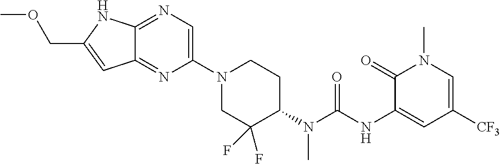 [SMILES: COCc1cc2nc(N3CC[C@H](N(C)C(=O)Nc4cc(C(F)(F)F)cn(C)c4=O)C(F)(F)C3)cnc2[nH]1] | |
| | (R)-1-(3,3-difluoro-1-(6- | |
| | (methoxymethyl)-5H-pyrrolo[2,3- | |
| | b]pyrazin-2-yl)piperidin-4-yl)-1-methyl- | |
| | 3-(1-methyl-2-oxo-5-(trifluoromethyl)- | |
| | 1,2-dihydropyridin-3-yl)urea | |
| | (S)-1-(3,3-difluoro-1-(6-(methoxymethyl)- | |
| | 5H-pyrrolo[2,3-b]pyrazin-2-yl)piperidin- | |
| | 4-yl)-1-methyl-3-(1-methyl-2-oxo-5- | |
| | (trifluoromethyl)-1,2-dihydropyridin-3- | |
| | yl)urea | |
| I-124 | 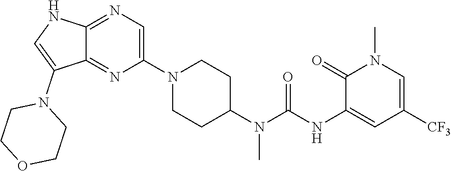 [SMILES: CN(C(=O)Nc1cc(C(F)(F)F)cn(C)c1=O)C1CCN(c2cnc3[nH]cc(N4CCOCC4)c3n2)CC1] | MS (ES) m/z 535.43 [M + H]₊ ₁H NMR (DMSO-d₆, 400 MHz): δ 10.95 (s, 1H), 8.17-8.16 (d, J = 2 Hz, 1H), 8.03 (s, 2H), 7.89 (s, 1H), 7.00-6.99 (d, J = 2.4 Hz, 1H), 4.34-4.31 (m, 3H) 4.32- 4.26 (m, 1H), 3.77 (s, 3H), 3.58 (s, 3H), 3.15 (m, 5H), 2.87-2.84 (m, 4H), 1.78- 1.76 (m, 2H), 1.73-1.68 (m, 2H) |
| | 1-methyl-3-(1-methyl-2-oxo-5- | |
| | (trifluoromethyl)-1,2-dihydropyridin-3- | |
| | yl)-1-(1-(7-morpholino-5H-pyrrolo[2,3- | |
| | b]pyrazin-2-yl)piperidin-4-yl)urea | |
| I-126- aª and I-126- b^{b} | 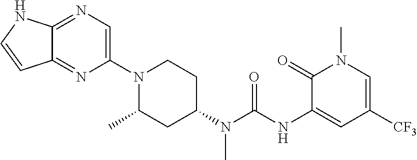 [SMILES: C[C@H]1C[C@@H](N(C)C(=O)Nc2cc(C(F)(F)F)cn(C)c2=O)CCN1c1cnc2[nH]ccc2n1] | MS (ES) m/z 464.43 [M + H]⁺ ¹H-NMR (400 MHz, DMSO-d₆): δ 11.64 (s, 1H), 8.20 (s, 1H), 8.05 (s, 1H), 8.01 (s, 1H), 7.93 (s, 1H), 7.57 (s, 1H), 6.31 (s, 1H), 4.84 (m, 1H), 4.56 (m, 1H), 4.23- 4.19 (m, 1H), 3.71 (s, 3H), 3.19-3.08 (m, 2H), 2.92 (s, 3H), 2.01-1.97 (m, 1H), 1.85-1.73 (m, 1H), 1.60-1.57 (m, 1H), 1.12-1.10 (d, J = 2.0 Hz, 3H) |
| | 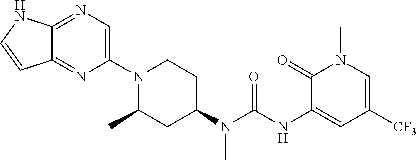 [SMILES: C[C@@H]1C[C@H](N(C)C(=O)Nc2cc(C(F)(F)F)cn(C)c2=O)CCN1c1cnc2[nH]ccc2n1] | |
| | 1-methyl-1-((2S,4S)-2-methyl-1-(5H- | |
| | pyrrolo[2,3-b]pyrazin-2-yl)piperidin-4- | |
| | yl)-3-(1-methyl-2-oxo-5- | |
| | (trifluoromethyl)-1,2-dihydropyridin-3- | |
| | yl)urea | |
| | 1-methyl-1-((2R,4R)-2-methyl-1-(5H- | |
| | pyrrolo[2,3-b]pyrazin-2-yl)piperidin-4- | |
| | yl)-3-(1-methyl-2-oxo-5- | |
| | (trifluoromethyl)-1,2-dihydropyridin-3- | |
| | yl)urea | |
| I-128- a^{g} and I-128- b^{h} | 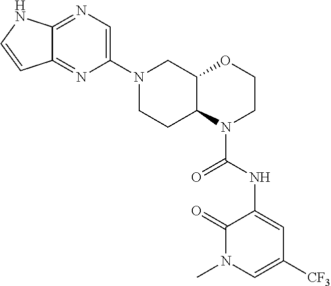 [SMILES: Cn1cc(C(F)(F)F)cc(NC(=O)N2CCO[C@H]3CN(c4cnc5[nH]ccc5n4)CC[C@@H]32)c1=O] | MS (ES) m/z 478.43 [M + H]⁺ ¹H NMR (DMSO-d₆, 400 MHz): δ 11.58 (s, 1H), 8.26 (s, 1H), 8.08 (s, 1H), 8.06 (s, 1H), 8.04 (s, 1H), 7.59 (s, 1H), 6.34 (s, 1H), 4.45-4.21 (m, 2H), 3.96-3.86 (m, 2H), 3.79-3.71 (m, 2H), 3.69 (s, 3H), 3.41-3.35 (m, 2H), 2.89-2.81 (m, 1H), 2.75-2.61 (m, 1H), 1.61-1.53 (m, 2H) |
| | 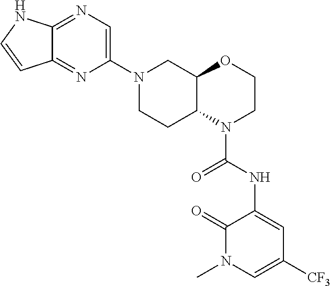 [SMILES: Cn1cc(C(F)(F)F)cc(NC(=O)N2CCO[C@@H]3CN(c4cnc5[nH]ccc5n4)CC[C@H]32)c1=O] | |
| | (4aS,8aS)-N-(1-methyl-2-oxo-5- | |
| | (trifluoromethyl)-1,2-dihydropyridin-3- | |
| | yl)-6-(5H-pyrrolo[2,3-b]pyrazin-2- | |
| | yl)octahydro-1H-pyrido[3,4- | |
| | b][1,4]oxazine-1-carboxamide | |
| | (4aR,8aR)-N-(1-methyl-2-oxo-5- | |
| | (trifluoromethyl)-1,2-dihydropyridin-3- | |
| | yl)-6-(5H-pyrrolo[2,3-b]pyrazin-2- | |
| | yl)octahydro-1H-pyrido[3,4- | |
| | b][1,4]oxazine-1-carboxamide | |
| I-149 | 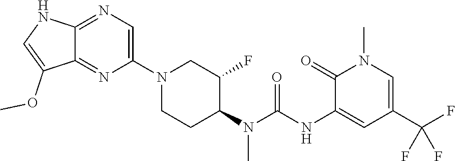 [SMILES: COc1c[nH]c2ncc(N3CC[C@H](N(C)C(=O)Nc4cc(C(F)(F)F)cn(C)c4=O)[C@@H](F)C3)nc12] | MS (ES) m/z 498.44 [M + H]⁺ ¹HNMR (DMSO-d₆, 400 MHz): δ 11.03 (s, 1H), 8.14(s, 1H), 8.05 (s, 1H), 7.98 (s, 1H), 7.24 (s, 1H), 4.90-4.76 (m, 1H), 4.74-4.67 (m, 1H), 4.42 (bs, 1H), 4.30- 4.27 (d, J = 12 Hz 1H), 3.89 (s, 3H), 3.59 (s, 3H), 3.01-2.89 (m, 5H), 1.88-1.77 (m, 2H) |
| | 1-((3S,4S)-3-fluoro-1-(7-methoxy-5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-150 | 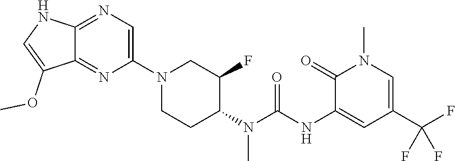 [SMILES: COc1c[nH]c2ncc(N3CC[C@@H](N(C)C(=O)Nc4cc(C(F)(F)F)cn(C)c4=O)[C@H](F)C3)nc12] | MS (ES) m/z 498.47 [M + H]⁺ ¹HNMR (DMSO-d₆, 400 MHz): δ 11.02 (s, 1H), 8.15 (s, 1H), 8.05 (s, 1H), 7.98 (s, 1H), 7.24 (s, 1H), 4.91-4.73 (m, 1H), 4.74-4.63 (m, 1H), 4.41 (bs, 1H), 4.30- 4.27 (d, J = 12 Hz 1H), 3.83 (s,3H), 3.59 (s, 3H), 3.01-2.89 (m, 5H), 1.88-1.77 (m, 2H) |
| | 1-((3R,4R)-3-fluoro-1-(7-methoxy-5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-151 | 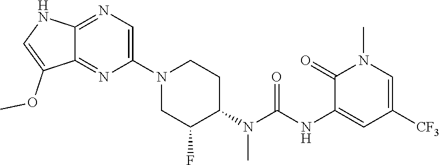 [SMILES: COc1c[nH]c2ncc(N3CC[C@H](N(C)C(=O)Nc4cc(C(F)(F)F)cn(C)c4=O)[C@H](F)C3)nc12] | MS (ES) m/z 498.47 [M + H]⁺ ¹H NMR (DMSO-d₆, 400 MHz): δ 10.95 (s, 1H), 8.19 (s, 1H), 8.08 (s, 1H), 8.06 (s, 1H), 7.98 (s, 1H), 7.21-7.20 (d, J = 4 Hz, 1H), 5.00-4.88 (m, 1H), 4.62-4.55 (m, 1H), 4.52-4.42 (m, 2H), 3.82 (s, 3H), 3.59 (s, 3H), 3.29-3.20 (m, 2H), 2.93 (s, 3H), 2.33-2.25 (m, 2H) |
| | 1-((3R,4S)-3-fluoro-1-(7-methoxy-5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-152 | 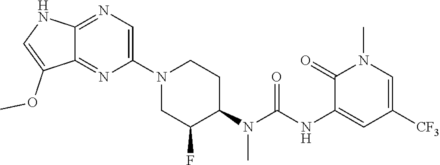 [SMILES: COc1c[nH]c2ncc(N3CC[C@@H](N(C)C(=O)Nc4cc(C(F)(F)F)cn(C)c4=O)[C@@H](F)C3)nc12] | MS (ES) m/z 498.47 [M + H]⁺ ¹H NMR (DMSO-d₆, 400 MHz): δ 10.94 (s, 1H), 8.18 (s, 1H), 8.08 (s, 1H), 8.05 (s, 1H), 7.97 (s, 1H), 7.21-7.20 (d, J = 4.0 Hz, 1H), 5.00-4.87 (d, J_{HF} = 50.0 Hz, 1H), 4.61-4.55 (m, 1H), 4.52-4.45 (m, 2H), 3.82 (s, 3H), 3.59 (s, 3H), 3.20-3.02 (m, 2H), 2.96 (s, 3H), 2.24-2.22 (m, 1H), 1.65-1.63 (m, 1H) |
| | 1-((3S,4R)-3-fluoro-1-(7-methoxy-5H- | |
| | pyrrolo[2,3-b]pyrazin-2-yl)piperidin-4- | |
| | yl)-1-methyl-3-(1-methyl-2-oxo-5- | |
| | (trifluoromethyl)-1,2-dihydropyridin-3- | |
| | yl)urea | |
| I-153-aⁱ and I- 153-b^{j} | 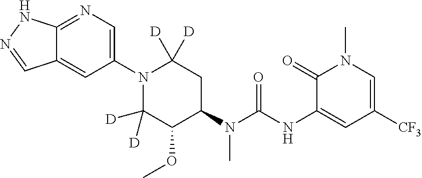 [SMILES: [2H]C1([2H])C[C@@H](N(C)C(=O)Nc2cc(C(F)(F)F)cn(C)c2=O)[C@H](OC)C([2H])([2H])N1c1cnc2[nH]ncc2c1] | MS (ES) m/z 484.50 [M + H]⁺ ¹H NMR (DMSO-d₆, 400 MHz): δ 13.09 (s, 1H), 8.49-8.50 (d, J = 2.8 Hz, 1H), 8.08 (s, 1H), 8.04 (s, 1H), 8.01 (s, 1H), 7.97 (s, 1H), 7.70 (s, 1H), 3.92 (s, 1H), 3.58 (s, 3H), 3.54 (m, 1H), 3.38 (s, 3H), 2.90 (s, 3H), 1.98-1.92 (m, 1H), 1.76 (m, 1H) |
| | 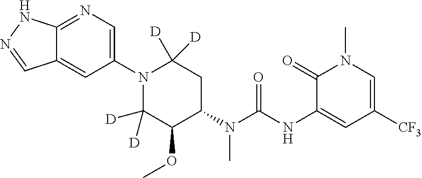 [SMILES: [2H]C1([2H])C[C@H](N(C)C(=O)Nc2cc(C(F)(F)F)cn(C)c2=O)[C@@H](OC)C([2H])([2H])N1c1cnc2[nH]ncc2c1] | |
| | 1-((3R,4R)-3-methoxy-1-(1H- | |
| | pyrazolo[3,4-b]pyridin-5-yl)piperidin-4- | |
| | yl-2,2,6,6-d₄)-1-methyl-3-(1-methyl-2- | |
| | oxo-5-(trifluoromethyl)-1,2- | |
| | dihydropyridin-3-yl)urea | |
| | 1-((3S,4S)-3-methoxy-1-(1H- | |
| | pyrazolo[3,4-b]pyridin-5-yl)piperidin-4- | |
| | yl-2,2,6,6-d₄)-1-methyl-3-(1-methyl-2- | |
| | oxo-5-(trifluoromethyl)-1,2- | |
| | dihydropyridin-3-yl)urea | |
| I-155- a^{k} and I-155-b^{l} | 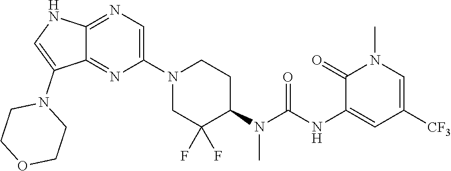 [SMILES: CN(C(=O)Nc1cc(C(F)(F)F)cn(C)c1=O)[C@@H]1CCN(c2cnc3[nH]cc(N4CCOCC4)c3n2)CC1(F)F] | MS (ES) m/z 571.36 [M + H]⁺ ¹H-NMR (400 MHz, DMSO-d₆): δ 11.06 (s, 1H), 8.15-8.05 (m, 3H), 7.04 (s, 1H), 4.89 (bs, 1H), 4.59-4.56 (t, J = 12.0 Hz, 1H), 4.43-4.40 (d, J = 12.0 Hz, 1H), 3.78 (s, 3H), 3.59 (s, 3H), 3.11-3.08 (m, 4H), 2.91-2.69 (m, 4H), 2.21-2.18 (m, 2H), 1.87 (m, 2H) |
| | 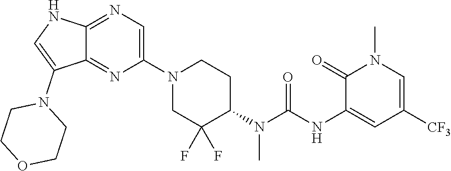 [SMILES: CN(C(=O)Nc1cc(C(F)(F)F)cn(C)c1=O)[C@H]1CCN(c2cnc3[nH]cc(N4CCOCC4)c3n2)CC1(F)F] | |
| | (R)-or (S)-1-(3,3-difluoro-1-(7- | |
| | morpholino-5H-pyrrolo[2,3-b]pyrazin-2- | |
| | yl)piperidin-4-yl)-1-methyl-3-(1-methyl- | |
| | 2-oxo-5-(trifluoromethyl)-1,2- | |
| | dihydropyridin-3-yl)urea | |
| I-161- a^{m} and I-161- bⁿ | 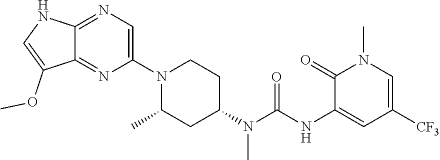 [SMILES: COc1c[nH]c2ncc(N3CC[C@H](N(C)C(=O)Nc4cc(C(F)(F)F)cn(C)c4=O)C[C@@H]3C)nc12] | MS (ES) m/z 494.26 [M + H]⁺ ¹H NMR (DMSO-d₆, 400 MHz): δ 11.24 (s, 1H), 8.17 (s, 1H), 8.04 (s, 1H), 7.98 (s, 1H), 7.92 (s, 1H), 7.33 (s, 1H), 4.21 (bs, 1H), 3.86 (s, 1H), 3.59 (s, 1H), 3.46- 3.45 (m, 1H), 3.18-3.12 (m, 2H), 2.92 (s, 3H), 1.84 (s, 1H), 1.74-1.68 (m, 3H), 1.01 (s, 3H) |
| | 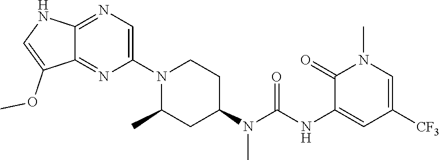 [SMILES: COc1c[nH]c2ncc(N3CC[C@@H](N(C)C(=O)Nc4cc(C(F)(F)F)cn(C)c4=O)C[C@H]3C)nc12] | |
| | 1-((2S,4S)-1-(7-methoxy-5H-pyrrolo[2,3- | |
| | b]pyrazin-2-yl)-2-methylpiperidin-4-yl)- | |
| | 1-methyl-3-(1-methyl-2-oxo-5- | |
| | (trifluoromethyl)-1,2-dihydropyridin-3- | |
| | yl)urea | |
| | 1-((2R,4R)-1-(7-methoxy-5H-pyrrolo[2,3- | |
| | b]pyrazin-2-yl)-2-methylpiperidin-4-yl)- | |
| | 1-methyl-3-(1-methyl-2-oxo-5- | |
| | (trifluoromethyl)-1,2-dihydropyridin-3- | |
| | yl)urea | |
| I-163- a^{e} and I-163- b^{f} | 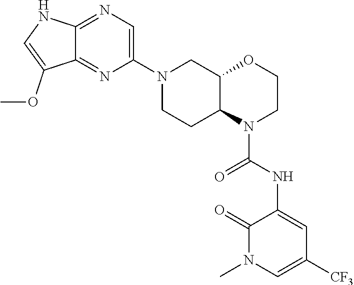 [SMILES: COc1c[nH]c2ncc(N3CC[C@H]4[C@H](C3)OCCN4C(=O)Nc3cc(C(F)(F)F)cn(C)c3=O)nc12] | MS (ES) m/z 508.14 [M + H]⁺ ¹H NMR (DMSO-d₆, 400 MHz): δ 12.25 (s, 1H), 8.41-8.40 (d, J = 2.8 Hz, 1H), 8.18-8.17 (d, J = 2.0 Hz, 1H), 8.13 (s, 1H), 8.03 (s, 1H), 7.45-7.44 (d, J = 2.4 Hz, 1H), 4.19-4.15 (m, 1H), 3.97 (s, 3H), 3.89-3.85 (m, 1H), 3.68-3.58 (m, 2H), 3.53 (s, 3H), 3.31 (s, 3H), 3.02 (s, 3H), 2.86-2.80 (m, 1H), 2.73-2.70 (m, 1H), 2.38-2.32 (m, 1H), 1.57-1.55 (m, 1H) |
| | 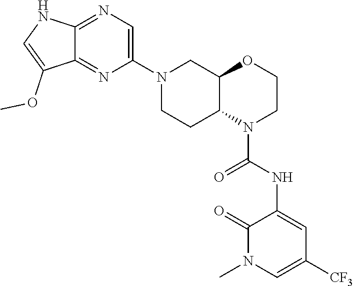 [SMILES: COc1c[nH]c2ncc(N3CC[C@@H]4[C@@H](C3)OCCN4C(=O)Nc3cc(C(F)(F)F)cn(C)c3=O)nc12] | |
| | (4aS,8aS)-6-(7-methoxy-5H-pyrrolo[2,3- | |
| | b]pyrazin-2-yl)-N-(1-methyl-2-oxo-5- | |
| | (trifluoromethyl)-1,2-dihydropyridin-3- | |
| | yl)octahydro-1H-pyrido[3,4- | |
| | b][1,4]oxazine-1-carboxamide | |
| | (4aR,8aR)-6-(7-methoxy-5H-pyrrolo[2,3- | |
| | b]pyrazin-2-yl)-N-(1-methyl-2-oxo-5- | |
| | (trifluoromethyl)-1,2-dihydropyridin-3- | |
| | yl)octahydro-1H-pyrido[3,4- | |
| | b][1,4]oxazine-1-carboxamide | |
| I-164- aª and I-164- b^{b} | 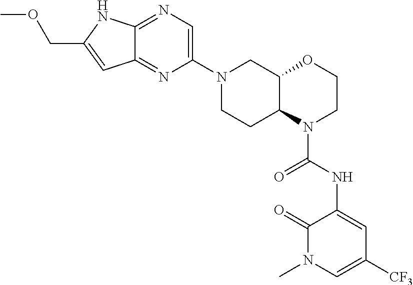 [SMILES: COCc1cc2nc(N3CC[C@H]4[C@H](C3)OCCN4C(=O)Nc3cc(C(F)(F)F)cn(C)c3=O)cnc2[nH]1] | MS (ES) m/z 522.32 [M + H]⁺ ¹H NMR (DMSO-d₆, 400 MHz): δ 11.68 (s, 1H), 8.27 (s, 1H), 8.09-8.08 (d, J = 2.8 Hz, 1H), 8.06-8.02 (m, 2H), 6.31- 6.30 (d, J = 2 Hz, 1H), 4.52 (s, 2H), 4.41- 4.37 (m, 1H), 4.29-4.25 (m, 1H), 3.96- 3.95 (m, 1H), 3.83-3.73 (m, 2H), 3.58 (m, 4H), 3.37 (m, 1H), 3.35 (s, 3H), 2.86- 2.80 (t, J = 10.4 Hz, 1H), 2.73-2.70 (m, 2H), 1.41-1.26 (m, 2H) |
| | 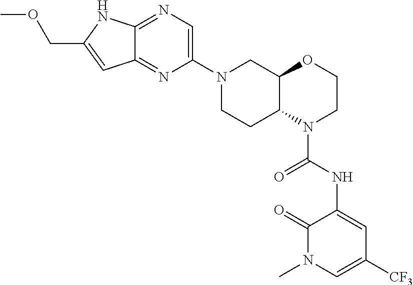 [SMILES: COCc1cc2nc(N3CC[C@@H]4[C@@H](C3)OCCN4C(=O)Nc3cc(C(F)(F)F)cn(C)c3=O)cnc2[nH]1] | |
| | (4aS,8aS)-6-(6-(methoxymethyl)-5H- | |
| | pyrrolo[2,3-b]pyrazin-2-yl)-N-(1-methyl- | |
| | 2-oxo-5-(trifluoromethyl)-1,2- | |
| | dihydropyridin-3-yl)octahydro-1/- | |
| | pyrido[3,4-b][1,4]oxazine-1-carboxamide | |
| | (4aR,8aR)-6-(6-(methoxymethyl)-5H- | |
| | pyrrolo[2,3-b]pyrazin-2-yl)-N-(1-methyl- | |
| | 2-oxo-5-(trifluoromethyl)-1,2- | |
| | dihydropyridin-3-yl)octahydro-1H- | |
| | pyrido[3,4-b][1,4]oxazine-1-carboxamide | |

ªSeparated by chiral SFC (Method D); first eluting isomer;
^{b}Separated by chiral SFC (Method D); second eluting isomer;
^{c}Separated by chiral HPLC (Method E); first eluting isomer;
^{d}Separated by chiral HPLC (Method E); second eluting isomer;
^{e}Separated by chiral SFC (Method F); first eluting isomer;
^{f}Separated by chiral SFC (Method F); second eluting isomer;
^{g}Separated by chiral HPLC (Method C); first eluting isomer;
^{h}Separated by chiral HPLC (Method C); second eluting isomer;
ⁱSeparated by chiral HPLC (Method K); first eluting isomer;
^{j}Separated by chiral HPLC (Method K); second eluting isomer;
^{k}Separated by chiral SFC (Method H); first eluting isomer;
^{l}Separated by chiral SFC (Method H); second eluting isomer.
^{m}Separated by chiral HPLC (Method M); first eluting isomer;
ⁿSeparated by chiral HPLC (Method M); second eluting isomer.

### Example 59: 1-(1-(7-cyano-5H-pyrrolo[2,3-b]pyrazin-2-yl)piperidin-4-yl)-1-methyl-3-(1-methyl-2-oxo-5-(trifluoromethyl)-1,2-dihydropyridin-3-yl)urea

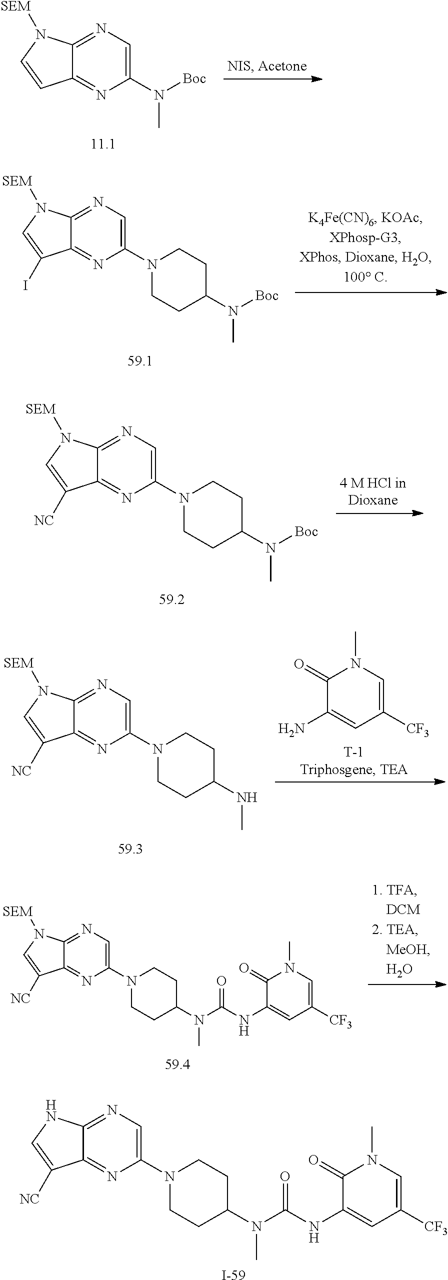

Synthesis of compound 59.1. To a solution of 11.1 (1.5 g, 3.25 mmol, 1.0 equiv) in acetone (20 mL) was added N-iodosuccinimide (1.09 g, 4.88 mmol, 1.5 equiv) in portions at 0° C. The reaction mixture was stirred at room temperature for 1 h. It was concentrated under reduce pressure. To the residue was added water and extracted with DCM. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford 59.1 (0.9 g, 42%). MS(ES): m/z 588.18 [M+H]⁺.

Synthesis of compound 59.2. To a solution of 59.1 (0.9 g, 1.53 mmol, 1.0 equiv) in 1,4-dioxane (8.0 mL) and water (2.0 mL) was degassed by bubbling through a stream of argon for 10 min. To the reaction mixture was added [2-dicyclohexylphosphino-2′,4′,6′-triisopropylbiphenyl] (0.036 g, 0.076 mmol, 0.05 equiv), methanesulfonato(2-dicyclohexylphosphino-2′,4′,6′-tri-1-propyl-1,1′-biphenyl)(2′-amino-1,1′-biphenyl-2-yl)palladium(II) (0.064 g, 0.076 mmol, 0.05 equiv), potassium ferrocyanide (0.323 g, 0.765 mmol, 0.5 equiv) and potassium acetate (0.030 g, 0.306 mmol, 0.2 equiv) and degassed for 5 min. The reaction mixture was stirred at 100° C. for 2 h. It was cooled to room temperature, filtered through a pad of Celite®. The filtrate was transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford 59.2 (0.35 g, 46.97%). MS(ES): m/z 487.28 [M+H]⁺.

Synthesis of compound 59.3. To a solution of 59.2 (0.35 g, 0.71 mmol, 1.0 equiv) in DCM (0.7 mL) was added 4 M hydrochloric acid in 1,4-dioxane (0.3 mL) dropwise at 0° C. The reaction mixture was stirred at room temperature for 1 h. It was concentrated under reduced pressure to afford 59.3 (0.25 g, 91%). MS(ES): m/z 387.23 [M+H]⁺.

Synthesis of compound 59.4. Compound 59.4 was prepared from compound 59.3 and T-1 following the procedure described in the synthesis of I-9. The product was purified by flash column chromatography on silica gel (CombiFlash®, 2.0% methanol in DCM). MS(ES): m/z 605.26 [M+H]⁺.

Synthesis of compound I-59. Compound I-59 was prepared from compound 59.4 following the procedure described in the synthesis of I-11. The product was purified by flash column chromatography on silica gel (CombiFlash®, 2.5% methanol in DCM). MS(ES): m/z 475.30 [M+H]⁺, ¹H-NMR (400 MHz, DMSO-d₆): δ 12.71 (s, 1H), 8.36 (s, 1H), 8.25 (s, 1H), 8.18-8.17 (d, J=2.0 Hz, 1H), 8.03 (s, 1H), 7.89 (s, 1H), 4.53-4.49 (m, 2H), 4.38-4.26 (m, 1H), 3.58 (s, 3H), 2.98-2.92 (m, 2H), 2.83 (s, 3H), 11.76-1.69 (m, 4H).

### Example 60: (S)-8-(7-cyano-5H-pyrrolo[2,3-b]pyrazin-2-yl)-3-methyl-N-(1-methyl-2-oxo-5-(trifluoromethyl)-1,2-dihydropyridin-3-yl)-1,8-diazaspiro[4.5]decane-1-carboxamide and (R)-8-(7-cyano-5H-pyrrolo[2,3-b]pyrazin-2-yl)-3-methyl-N-(1-methyl-2-oxo-5-(trifluoromethyl)-1,2-dihydropyridin-3-yl)-1,8-diazaspiro[4.5]decane-1-carboxamide

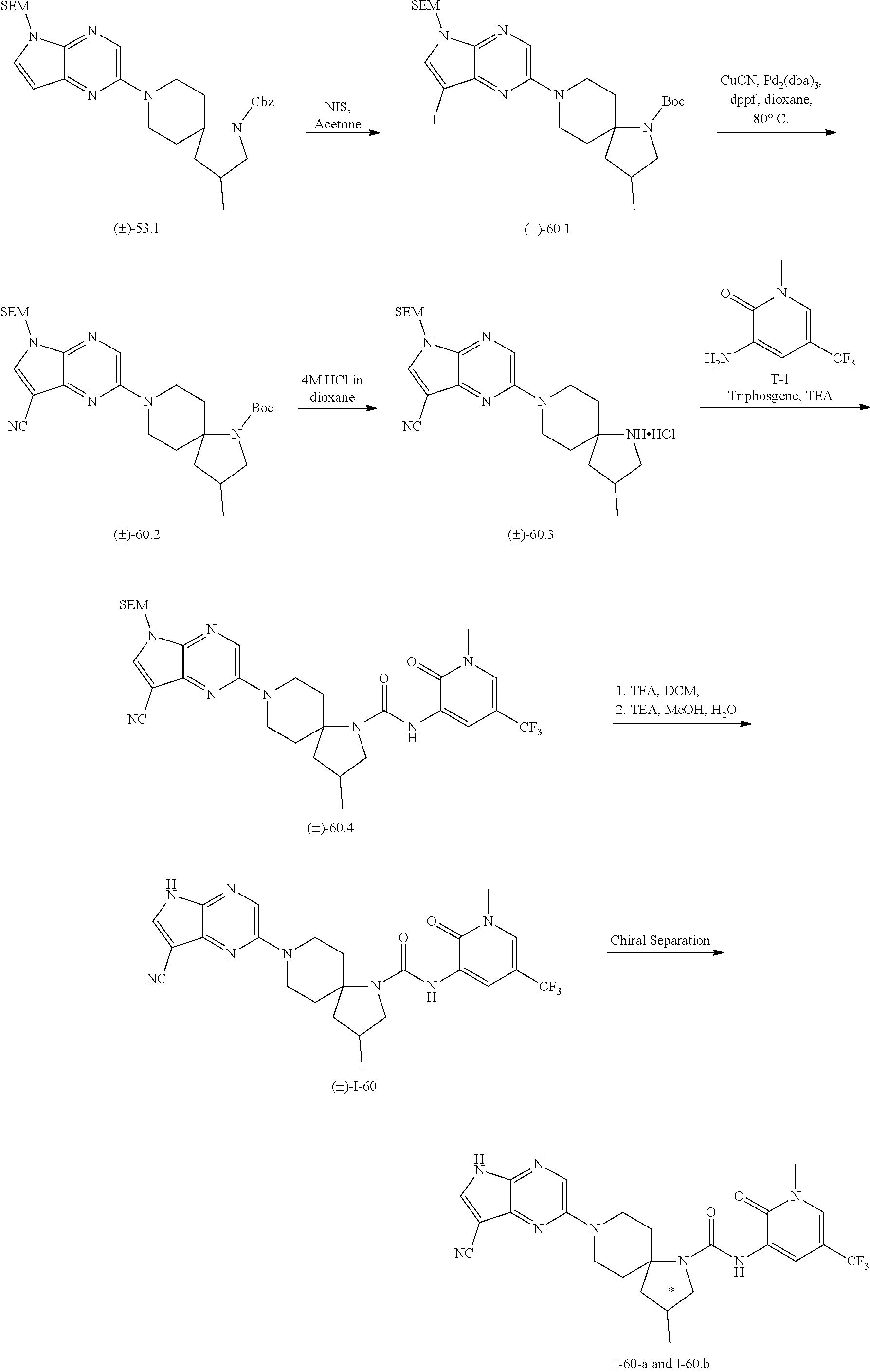

Synthesis of compound (±)-60.1. To a solution of (±)-53.1 (0.42 g, 0.83 mmol, 1.0 equiv) in acetone (8.0 mL) was added N-iodosuccinimide (0.281 g, 1.25 mmol, 1.5 equiv) in portions at 0° C. The reaction mixture was stirred at room temperature for 1 h. It was concentrated under reduce pressure. To the residue was added water and extracted with DCM. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford (±)-60.1 (0.37 g, 71%). MS(ES): m/z 628.21 [M+H]⁺.

Synthesis of compound (±)-60.2. A mixture of (±)-60.1 (0.37 g, 0.59 mmol, 1.0 equiv), 1,4-dioxane (5.0 mL) and copper(I) cyanide (0.210 g, 2.36 mmol, 4.0 equiv) was degassed by bubbling through a stream of argon for 10 min. Tris(dibenzylideneacetone)dipalladium(O) (0.027 g, 0.029 mmol, 0.05 equiv) and 1,1′-bis(diphenylphosphino)ferrocene (0.033 g, 0.059 mmol, 0.1 equiv) were added and degassed for 10 min. The reaction mixture was stirred at 80° C. for 16 h. It was cooled to room temperature, filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 18% ethyl acetate in hexane) to afford (f)-60.2 (0.2 g, 64%). MS(ES): m/z 527.31 [M+H]⁺.

Synthesis of compound (±)-60.3. To a solution of (±)-60.2 (0.2 g, 0.37 mmol, 1.0 equiv) in 1,4-dioxane (0.8 mL) was added 4 M hydrochloric acid in 1,4-dioxane (0.9 mL) dropwise at 0° C. The reaction mixture was stirred at room temperature for 1 h. It was concentrated under reduced pressure. The residue was used in the next step without further purification (±)-60.3 (0.170 g, 99%). MS(ES): m/z 463.23 [M+H]⁺.

Synthesis of compound (±)-60.4. Compound (±)-60.4 was prepared from compound (±)-60.4 and T-1 following the procedure described in the synthesis of I-9. The product was purified by flash column chromatography on silica gel (CombiFlash®, 3.5% methanol in DCM). MS(ES): m/z 645.29 [M+H]⁺.

Synthesis of compound (±)-I-60. Compound (±)-I-60 was prepared from compound (±)-60.4 following the procedure described in the synthesis of I-11. The product was purified by flash column chromatography on silica gel (CombiFlash®, 3.2% methanol in DCM). MS(ES): m/z 515.21 [M+H]⁺.

I-60-a and I-60-b. The racemate was separated by chiral SFC (Method H) to afford first eluting fraction (I-60-a) and second eluting fraction (I-60-b). *Absolute stereochemistry was not determined.

I-60-a. MS(ES): m/z: 515.21 [M+H]⁺, ¹H-NMR (400 MHz, DMSO-d₆): δ 12.59 (s, 1H), 8.37-8.36 (d, J=2.4 Hz, 1H), 8.24-8.23 (d, J=2.4 Hz, 1H), 8.04 (s, 1H), 7.99 (s, 1H), 7.57 (s, 1H), 4.45-4.33 (m, 2H), 3.68-3.66 (m, 1H), 3.57 (s, 3H), 3.14-3.09 (m, 1H), 3.05-2.85 (m, 4H), 2.37-2.33 (m, 1H), 1.55-1.52 (m, 1H), 1.47-1.32 (m, 3H), 1.10-1.09 (d, J=6.0 Hz, 3H).

I-60-b. MS(ES): m/z: 515.44 [M+H]⁺, ¹H-NMR (400 MHz, DMSO-d₆): δ 12.59 (s, 1H), 8.36 (s, 1H), 8.23 (s, 1H), 8.04 (s, 1H), 7.99 (s, 1H), 7.57 (s, 1H), 4.45-4.33 (m, 2H), 3.68-3.66 (m, 1H), 3.57 (s, 3H), 3.13-3.09 (m, 1H), 3.01-2.89 (m, 4H), 2.41-2.33 (m, 1H), 1.55-1.52 (m, 1H), 1.47-1.32 (m, 3H), 1.10-1.08 (d, J=6.4 Hz, 3H).

### Example 65: 1-(1-(7-fluoro-5H-pyrrolo[2,3-b]pyrazin-2-yl)piperidin-4-yl)-N-(1-methyl-2-oxo-5-(trifluoromethyl)-1,2-dihydropyridin-3-yl)cyclopropane-1-carboxamide

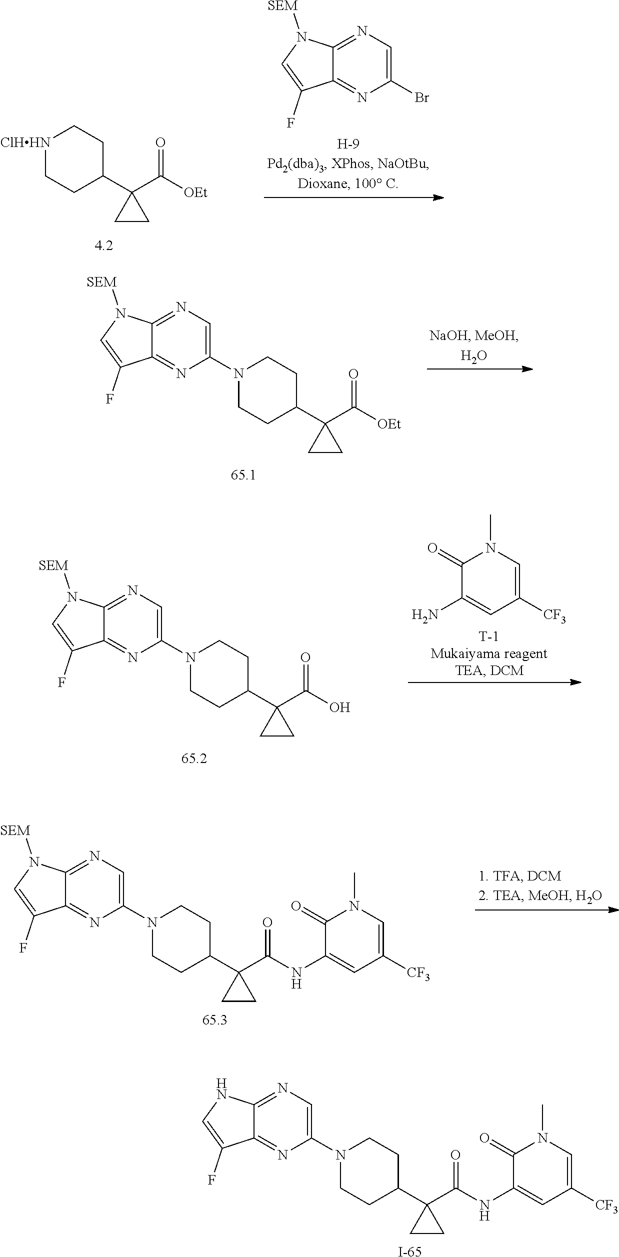

Synthesis of compound 65.1. Compound 65.1 was prepared from compound 4.2 and H-9 following the procedure described in the synthesis of compound 11.1. The product was purified by flash column chromatography on silica gel (CombiFlash®, 20% ethyl acetate in hexane). MS (ES): m/z 463.25 [M+H]⁺.

Synthesis of compound 65.2. To a stirred solution of 65.1 (0.9 g, 7.70 mmol, 1.0 equiv) in methanol was added an aqueous solution of sodium hydroxide (0.389, 9.7310 mmol, 5.0 equiv) dropwise. The reaction mixture was stirred at 60° C. for 3 h. It was concentrated under reduced pressure. To the residue was added water and acidified with 1 N hydrochloric acid to adjust to pH 6.5 at 10° C. The mixture was extracted with 10% methanol in DCM. The combined organic layers were washed with brine, dried over anhydrous sodium sulfate, filtered, and concentrated under reduced pressure to afford 65.2 (0.35 g, 41%). MS (ES): m/z 435.21 [M+H]⁺.

Synthesis of compound 65.3. To a solution of 65.2 (0.35 g, 0.850 mmol, 1.0 equiv) and triethylamine (0.244 g, 2.41 mmol, 3.0 equiv) in DCM (5.0 mL) was added a 2-chloro-1-methylpyridinium iodide (Mukaiyama reagent, 0.246 g, 0.96 mmol, 1.2.0 equiv) and stirred for 48 h. It was added to water and extracted with ethyl acetate. The organic layer was separated, dried over anhydrous sodium sulfate, and concentrated under reduced pressure to afford the crude product. The residue was purified by flash column chromatography on silica gel (CombiFlash®, 5% methanol in DCM) to afford 65.3 (0.06 g, 12%). MS (ES): m/z 609.71 [M+H]⁺.

Synthesis of compound I-65. Compound I-65 was prepared from compound 65.3 following the procedure described in the synthesis of compound I-11. The product was purified by flash column chromatography on silica gel (CombiFlash®, 3% methanol in DCM). MS(ES): m/z 479.49 [M+H]⁺, ¹H NMR (DMSO-d₆, 400 MHz): δ 11.36 (s, 1H), 8.76 (s, 1H), 8.27 (s, 1H), 8.14 (s, 1H), 8.12 (s, 1H), 7.56 (s, 1H), 4.38-4.35 (m, 2H), 3.58 (s, 3H), 2.82-2.76 (m, 2H), 1.91-1.83 (m, 1H), 1.81-1.75 (m, 2H), 1.41-1.38 (m, 2H), 0.97 (s, 2H), 0.87 (s, 2H).

### Example 66: 1-(1-(isothiazolo[5,4-b]pyridin-5-yl)piperidin-4-yl)-1-methyl-3-(1-methyl-2-oxo-5-(trifluoromethyl)-1,2-dihydropyridin-3-yl)urea

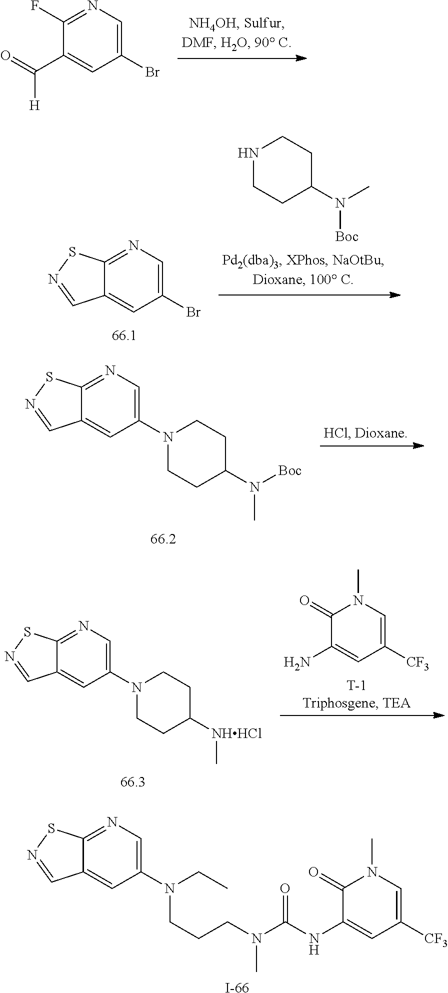

Synthesis of compound 66.1. A mixture of 5-bromo-2-fluoronicotinaldehyde (7.0 g, 14.0 mmol, 1.0 equiv) and sulfur (10.56 g, 41.18 mmol, 5 equiv) in DMF (80 mL) was added at 0° C. was added an aqueous solution of ammonium hydroxide (70 mL). The reaction mixture was stirred at 90° C. in a sealed autoclave for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (60% ethyl acetate in hexane) to afford 66.1 (0.650 g, 8.8%). MS(ES): m/z 216.92 [M+H]⁺.

Synthesis of compound 66.2. Compound 66.2 was prepared from compound 66.1 and tert-butyl methyl(piperidin-4-yl)carbamate following the procedure described in the synthesis of compound 11.1. The product was purified by flash column chromatography on silica gel (CombiFlash®, 40% ethyl acetate in hexane). MS(ES): m/z 349.16 [M+H]⁺.

Synthesis of compound 66.3. To a solution of 66.2 (0.13 g, 0.373 mmol, 1.0 equiv) in DCM (5 mL) was added 4.0 M hydrochloric acid in dioxane (2.8 mL) at 0° C. The reaction mixture was stirred at room temperature for 3 h. It was concentrated under reduced pressure to afford 66.3 (0.1 g, 94%). MS(ES): m/z 249.11 [M+H]⁺.

Synthesis compound I-66. Compound I-66 was prepared from compound 66.3 and T-1 following the procedure described in the synthesis of 1-9. The product was purified by flash column chromatography on silica gel (2.5% methanol in DCM). MS(ES): m/z 467.20 [M+H]*, H NMR (DMSO-d₆, 400 MHz): δ 9.02 (s, 1H), 8.79-8.78 (d, J=2.4 Hz, 1H), 8.18-8.17 (d, J=2.4 Hz, 1H), 8.03 (s, 1H), 8.01-8.00 (d, J=2.8 Hz, 1H) 7.91 (s, 1H), 4.28-4.22 (m, 1H), 3.95-3.92 (d, J=12.0 Hz, 2H) 3.58 (s, 3H), 2.93-2.88 (m, 2H), 2.90 (s, 3H), 1.92-1.84 (m, 2H), 1.71-1.68 (m, 2H).

### Example 69: 1-(1-(3-methoxy-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

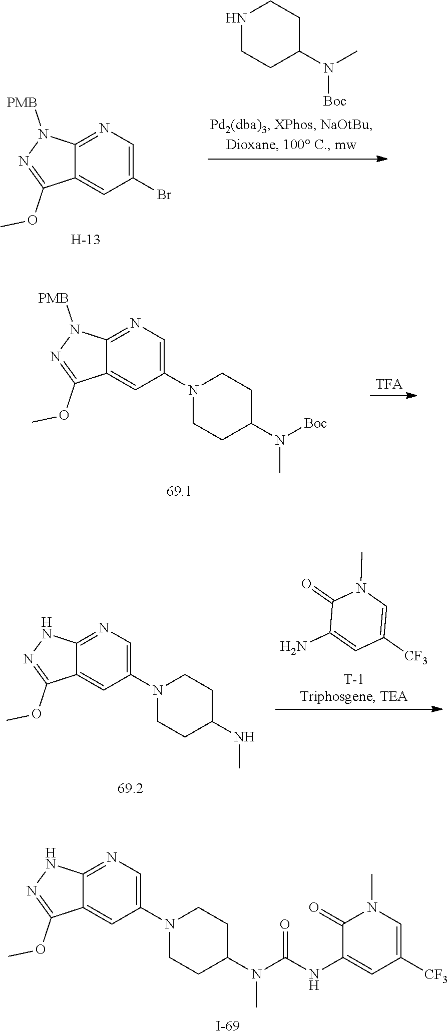

Synthesis of compound 69.1. Compound 69.1 was prepared from compound H-13 and tert-butyl methyl(piperidin-4-yl)carbamate following the procedure described in the synthesis of compound 11.1. The product was purified by flash column chromatography on silica gel (CombiFlash®, 40% ethyl acetate in hexane). MS(ES): m/z: 482.17 [M+H]⁺.

Synthesis of compound 69.2. To a solution of 69.1 (0.410 g, 0.851 mmol, 1.0 equiv) in trifluoroacetic acid (5.0 mL) was stirred at 80° C. for 6 h. It was poured over aqueous solution of sodium bicarbonate and extracted with ethyl acetate. The combined organic layers were washed with brine, dried over anhydrous sodium sulfate, filtered, and concentrated under reduced pressure. The residue was purified by flash column chromatography on silica gel (2.5% methanol in DCM) to afford 69.2 (0.17 g, 76%). MS(ES): m/z 262.33 [M+H]⁺.

Synthesis of compound I-69. Compound I-69 was prepared from compound 69.2 and T-1 following the procedure described in the synthesis of I-9. The product was purified by flash column chromatography on silica gel (2% methanol in DCM). MS(ES): m/z 480.24 [M+H]⁺, ¹H NMR (DMSO-d₆, 400 MHz): δ 12.27 (s, 1H), 8.42 (s, 1H), 8.18 (s, 1H), 8.04 (s, 1H), 7.91 (s, 1H), 7.47 (s, 1H), 4.22-4.13 (m, 1H), 3.98 (s, 3H), 3.66-3.63 (m, 2H), 3.59 (s, 3H), 2.90 (s, 3H), 2.79-2.73 (m, 2H), 1.92-1.90 (m, 2H), 1.69-1.67 (m, 2H).

### Example 71: 4-methyl-1-(5H-pyrrolo[2,3-b]pyrazin-2-yl)piperidin-4-yl (1-methyl-2-oxo-5-(trifluoromethyl)-1,2-dihydropyridin-3-yl)carbamate

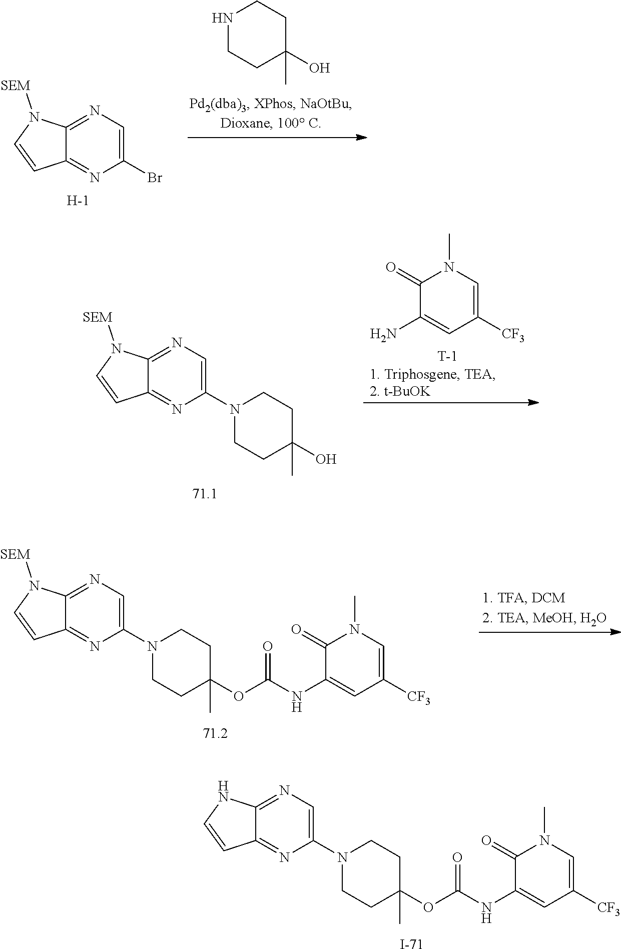

Synthesis of compound 71.1. Compound 71.1 was prepared from compound H-1 and 4-methylpiperidin-4-ol following the procedure described in the synthesis of compound 11.1. The product was purified by flash column chromatography on silica gel (CombiFlash®, 30% ethyl acetate in hexane) to afford 71.1 (0.93 g, 84%). MS (ES): m/z 363.21 [M+H]⁺.

Synthesis of compound 71.2. To a solution of compound 71.1 (0.293 g, 1.519 mmol, 1.0 equiv) in THE (5 mL) was added triethylamine (0.86 mL, 6.104 mmol, 4.0 equiv) at 0° C. Triphosgene (0.225 g, 0.758 mmol, 0.5 equiv) solution in THF (0.5 mL) was added dropwise and stirred for 15 min. Potassium tert-butoxide (0.85 g, 7.596 mmol, 5.0 equiv) and T-1 (0.550 g, 1.519 mmol, 1.0 equiv) were added and the reaction mixture was stirred at room temperature for 1.5 h. It was transferred into water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.6% methanol in DCM) to afford 71.2 (0.41 g, 47%). MS(ES): m/z 581.24 [M+H]⁺.

Synthesis of compound I-71. Compound I-71 was prepared from compound 71.2 following the procedure described in the synthesis of I-11. The product was purified by flash column chromatography on silica gel (CombiFlash®, 2.2% methanol in DCM). MS(ES): m/z 451.34 [M+H]⁺, ¹H NMR (DMSO-d₆, 400 MHz): δ 11.53 (s, 1H), 8.54 (s, 1H), 8.09 (s, 1H), 8.04 (s, 1H), 8.00 (s, 1H), 7.57 (s, 1H), 6.31 (s, 1H), 3.94-3.90 (m, 2H), 3.56 (s, 3H), 3.38-3.26 (m, 2H), 2.24-2.20 (m, 2H), 1.76-1.71 (m, 2H), 1.57 (s, 3H).

### Example 75: 1-(2-(7-cyano-5H-pyrrolo[2,3-b]pyrazin-2-yl)-2-azaspiro[3.3]heptan-6-yl)-1-methyl-3-(1-methyl-2-oxo-5-(trifluoromethyl)-1,2-dihydropyridin-3-yl)urea

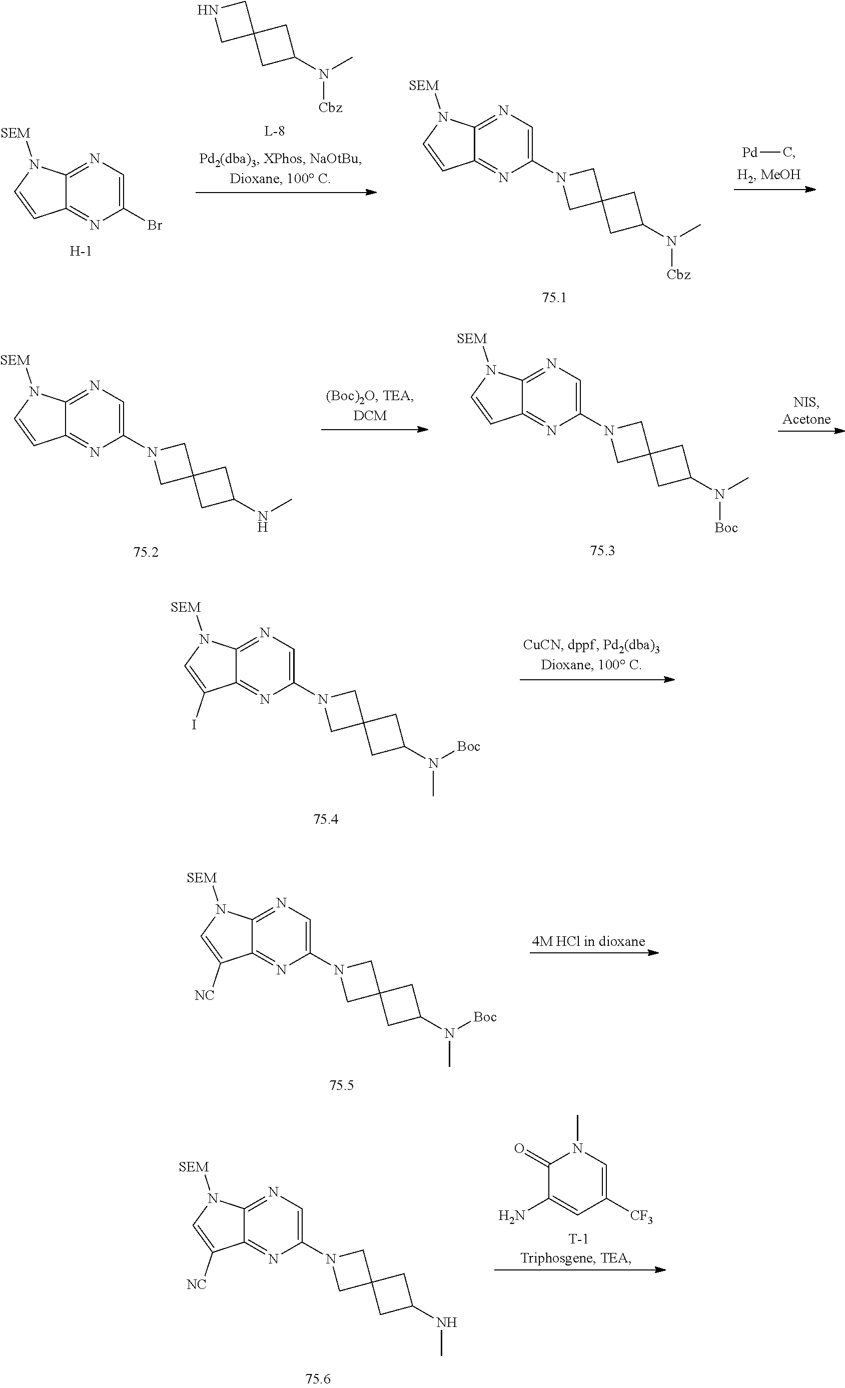

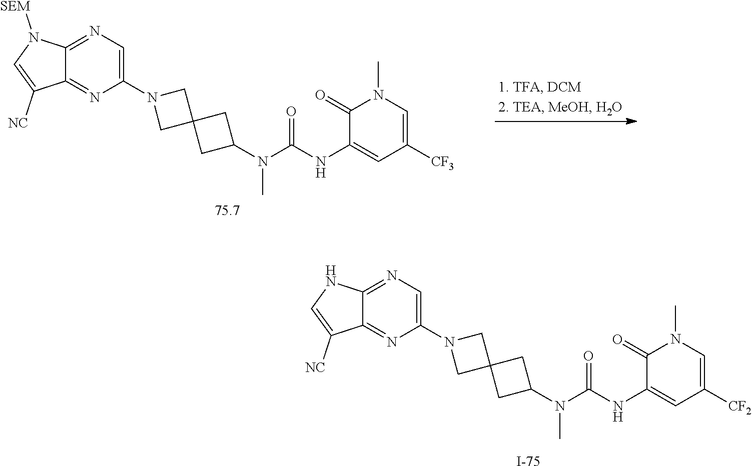

Synthesis of compound 75.1. Compound 75.1 was prepared from compound H-1 and L-8 following the procedure described in the synthesis of compound 11.1. The product was purified by flash column chromatography on silica gel (CombiFlash®, 2.0% methanol in DCM). MS(ES): m/z: 508.27 [M+H]⁺.

Synthesis of compound 75.2. A mixture of 75.1 (0.630 g, 1.24 mmol, 1.0 equiv), methanol (6.0 mL) and 10% palladium on carbon (0.6 g) was stirred under hydrogen atmosphere for 7 h. The reaction mixture was filtered through a pad of Celite® and rinsed with methanol. The filtrate was concentrated under reduced pressure to afford 75.2 (0.47 g, 100%). MS(ES): m/z 374.23 [M+H]⁺.

Synthesis of compound 75.3. To a solution of 75.2 (0.47 g, 1.25 mmol, 1.0 equiv) in DCM (5.0 mL) at 0° C. was added triethylamine (0.45 mL, 3.14 mmol, 2.5 equiv) followed by dropwise addition of di-tert-butyl dicarbonate (0.45 mL, 1.87 mmol, 1.5 equiv). The reaction was stirred at room temperature for 2 h. It was transferred into ice, stirred, and extracted with DCM. The combined organic layers were washed with brine, dried over anhydrous sodium sulfate, filtered, and concentrated under reduced pressure to afford 75.3 (0.425 g, 72%). MS (ES): m/z 474.28 [M+H]⁺.

Synthesis of compound 75.4. To a solution of 75.3 (0.425 g, 0.89 mmol, 1.0 equiv) in acetone (10 mL) was added N-iodosuccinimide (0.301 g, 1.34 mmol, 1.5 equiv in portions) at 0° C. The reaction mixture was stirred at room temperature for 1 h. It was concentrated under reduced pressure. To the residue was added water and extracted with DCM. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford 75.4 (0.348 g, 65%). MS(ES): m/z 600.18 [M+H]⁺.

Synthesis of compound 75.5. A solution of 75.4 (0.348 g, 0.58 mmol, 1.0 equiv) in 1,4-dioxane (4.0 mL) was degassed by bubbling through a stream of argon for 10 min. To the reaction mixture was added tris(dibenzylideneacetone) dipalladium (O) (0.027 g, 0.029 mmol, 0.05 equiv), 1,1′-bis(diphenylphosphino)ferrocene (0.031 g, 0.058 mmol, 0.1 equiv) and copper(I) cyanide (0.258 g, 2.90 mmol, 5.0 equiv) and degassed for 5 min. The reaction mixture was stirred at 100° C. for 5 h. It was cooled to room temperature, filtered through a pad of Celite®. The filtrate was transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford 75.5 (0.20 g, 69%). MS(ES): m/z 499.28 [M+H]⁺.

Synthesis of compound 75.6. To a solution of 75.5 (0.20 g, 0.40 mmol, 1.0 equiv) in DCM (1.0 mL) was added 4.0 M hydrochloric acid in dioxane (0.5 mL). The reaction mixture was stirred at room temperature for 2 h. It was concentrated under reduced pressure. The residue was azeotroped with DCM three time to afford 75.6 (0.145 g, 89%). MS(ES): m/z 399.23 [M+H]⁺.

Synthesis of compound 75.7. Compound 75.7 was prepared from compound 75.6 and T-1 following the procedure described in the synthesis of I-9. The product was purified by flash column chromatography on silica gel (3.0% methanol in DCM). MS(ES): m/z 617.26 [M+H]⁺.

Synthesis of compound I-75. Compound I-75 was prepared from compound 75.7 following the procedure described in the synthesis of I-11. The product was purified by flash column chromatography on silica gel (CombiFlash®, 3.5% methanol in DCM). MS(ES): m/z 487.17 [M+H]⁺, ¹H-NMR (400 MHz, DMSO-d₆): δ 9.16 (s, 1H), 8.15-8.14 (d, J=2.4 Hz, 1H), 8.02 (s, 1H), 7.99 (s, 1H), 7.84 (s, 1H), 7.80 (s, 1H), 4.78-4.76 (m, 1H), 3.57 (s, 4H), 3.53 (s, 3H), 2.96 (s, 3H), 2.36-2.33 (m, 2H), 2.23-2.21 (m, 2H).

### Example 76: 1-methyl-3-(1-methyl-2-oxo-5-(trifluoromethyl)-1,2-dihydropyridin-3-yl)-1-(1-(2-oxo-2,3-dihydro-1H-imidazo[4,5-b]pyridin-6-yl)piperidin-4-yl)urea

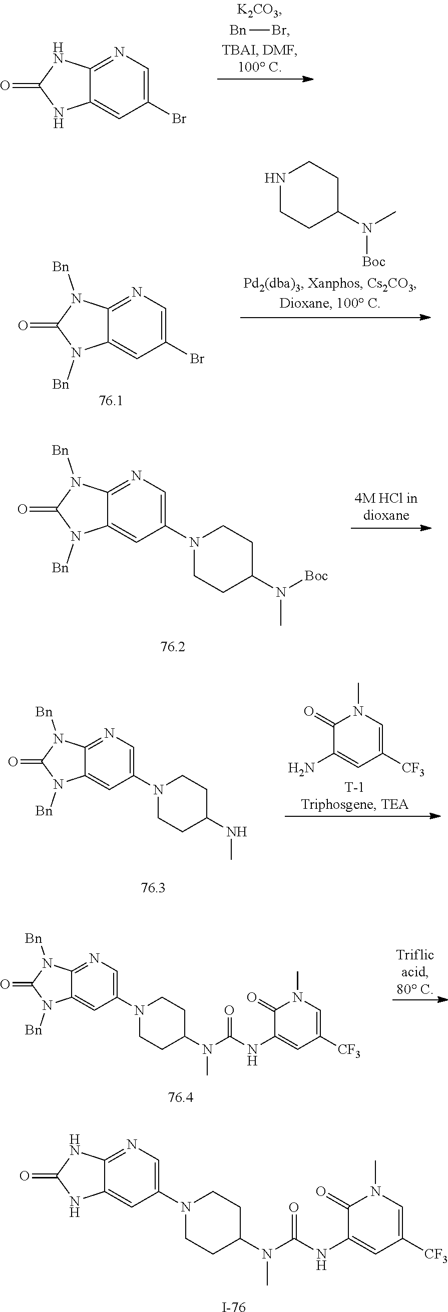

Synthesis of compound 76.1. To a mixture of 6-bromo-1,3-dihydro-2H-imidazo[4,5-b]pyridin-2-one (2.0 g, 9.34 mmol, 1.0 equiv) and potassium carbonate (0.727 g, 55.55 mmol, 3.0 equiv) in DMF (50 mL) was added tetra-n-butyl ammonium iodide (3.7 g, 10.27 mmol, 1.1 equiv) in portions at 0° C. and the reaction mixture was stirred for 10 min. To the mixture was added benzyl bromide (2.4 mL, 20.56 mmol, 2.2 equiv) and stirred at 100° C. for 16 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (CombiFlash®, 30% ethyl acetate in hexane) to afford 76.1 (2.0 g, 54%), MS(ES): m/z 394.05 [M+H]⁺.

Synthesis of compound 76.2. A mixture of 76.1 (1.0 g, 2.53 mmol, 1.0 equiv), tert-butyl methyl(piperidin-4-yl)carbamate (2.7 g, 12.65 mmol, 5.0 equiv), cesium carbonate (2.4 g, 7.61 mmol, 3.0 equiv) and 4,5-bis(diphenylphosphino)-9,9-dimethylxanthene (0.292 g, 0.506 mmol, 0.2 equiv) in 1,4-dioxane (5 mL) was degassed by bubbling through a stream of argon for 10 min. Tris(dibenzylideneacetone)dipalladium(O) (0.231 g, 0.253 mmol, 0.1 equiv) was added and the reaction mixture was stirred at 100° C. for 1 h in a microwave reactor. It was cooled to room temperature, poured over water, and extracted with ethyl acetate. The organic layer was separated, dried over anhydrous sodium sulfate, filtered, and concentrated under reduced pressure. The residue was purified by flash column chromatography on silica gel (CombiFlash®, 3.0% methanol in DCM) to afford 76.2 (0.400 g, 30%). MS(ES): m/z: 528.29 [M+H]⁺.

Synthesis of compound 76.3. To a solution of 76.2 (0.4 g, 0.75 mmol, 1.0 equiv) in DCM (1.0 mL) was added hydrochloric acid (4.0 M in dioxane) (0.5 mL). The reaction mixture was stirred at room temperature for 2 h. It was concentrated under reduced pressure. The residue was azeotroped with DCM three times to afford 76.3 (0.350 g, 100%). MS(ES): m/z 428.24 [M+H]⁺.

Synthesis of compound 76.4. Compound 76.4 was prepared from compound 76.3 and T-1 following the procedure described in the synthesis of I-9. The product was purified by flash column chromatography on silica gel (3.0% methanol in DCM). MS(ES): m/z 646.27 [M+H]⁺.

Synthesis of compound 1-76. To a solution of 76.4 (0.105 g, 0.16 mmol, 1.0 equiv) in DCM (3.0 mL) was cooled to 0° C. and added trifluoromethanesulfonic acid (1.0 mL). The reaction mixture was stirred at 80° C. for 1 h. It was concentrated under reduced pressure. The residue was diluted with DCM (10 mL), basified with saturated sodium bicarbonate solution and extracted with DCM. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford 1-76 (0.025 g, 33%). MS(ES): m/z 466.41 [M+H]⁺, ¹H-NMR (400 MHz, DMSO-d₆): δ 11.00 (s, 1H), 10.68 (s, 1H), 8.18 (s, 1H), 8.04 (s, 1H), 7.91 (s, 1H), 7.60 (s, 1H), 6.95 (s, 1H), 4.21-4.12 (m, 1H), 3.60 (s, 3H), 2.90 (s, 3H), 2.76-2.68 (m, 2H), 2.65-2.54 (m, 2H), 1.88-1.86 (m, 2H), 1.67-1.65 (m, 2H).

### Example 78: N-(1-methyl-2-oxo-5-(trifluoromethyl)-1,2-dihydropyridin-3-yl)-8-(5H-pyrrolo[2,3-b]pyrazin-2-yl)-1,8-diazaspiro[4.5]dec-3-ene-1-carboxamide

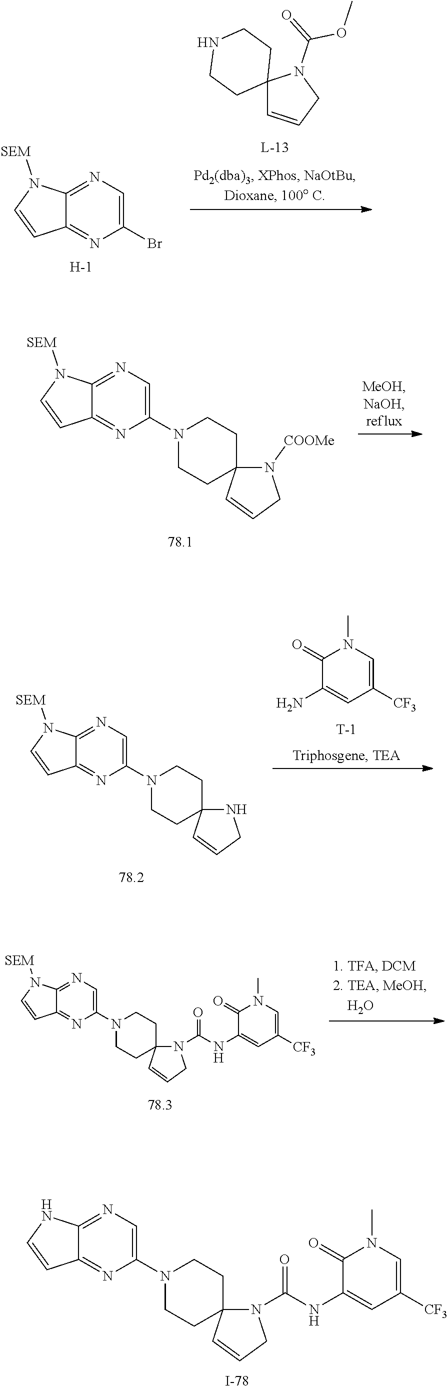

Synthesis of compound 78.1. Compound 78.1 was prepared from compound H-1 and L-13 following the procedure described in the synthesis of compound 11.1. The product was purified by flash column chromatography on silica gel (CombiFlash®, 35% ethyl acetate in hexane) to afford ed 2.1 (0.220 g, 46.80%). MS(ES): m/z: 444.24 [M+H]⁺.

Synthesis of compound 78.2. To a solution of 78.1 (0.170 g, 0.38 mmol, 1.0 equiv) in methanol (3.0 mL) was added sodium hydroxide (0.306 g, 7.65 mmol, 20 equiv) in portions. The reaction mixture was stirred at 90° C. for 16 h. It was quenched with water and extracted with 10% methanol in DCM. The combined organic layers were washed with brine, dried over anhydrous sodium sulfate, filtered, and concentrated under reduced pressure. The residue was purified by flash column chromatography on silica gel (CombiFlash®, 3.5% methanol in DCM) to afford 78.2. (0.110 g, 75%). MS(ES): m/z 386.23 [M+H]⁺.

Synthesis of compound 78.3. Compound 78.3 was prepared from compound 78.2 and T-1 following the procedure described in the synthesis of I-9. The product was purified by flash column chromatography on silica gel (3.0% methanol in DCM). MS(ES): m/z 604.26 [M+H]⁺.

Synthesis of I-78. Compound I-78 was prepared from compound 78.3 following the procedure described in the synthesis of I-11. The product was purified by flash column chromatography on silica gel (CombiFlash®, 3.8% methanol in DCM). MS(ES): m/z 474.42 [M+H]⁺, ¹H NMR (DMSO-d₆, 400 MHz): δ 11.55 (s, 1H), 8.07-8.05 (d, J=5.2 Hz, 2H), 8.01 (s, 1H), 7.61 (s, 1H), 7.58 (s, 1H), 6.62-6.60 (d, J=6.0 Hz, 1H), 6.32 (s, 1H), 5.96-5.95 (d, J=6.0 Hz, 1H), 4.36 (s, 2H), 4.32-4.29 (m, 2H), 3.57 (s, 3H), 3.08-3.01 (m, 2H), 2.96-2.90 (m, 2H), 1.49-1.46 (m, 2H).

The following compounds were prepared following the procedure of I-78:

| Cmpd. | | |
|---|---|---|
| No. | Structure and Name | Analytical Data |
| I-81 | 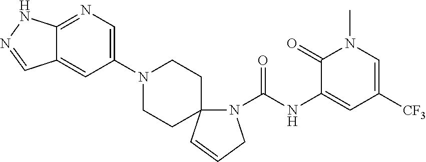 [SMILES: Cn1cc(C(F)(F)F)cc(NC(=O)N2CC=CC23CCN(c2cnc4[nH]ncc4c2)CC3)c1=O] N-(1-methyl-2-oxo-5-(trifluoromethyl)- 1,2-dihydropyridin-3-yl)-8-(1H- pyrazolo[3,4-b]pyridin-5-yl)-1,8- diazaspiro[4.5]dec-3-ene-1- carboxamide | MS (ES): m/z 474.38 [M + H]⁺ ¹H NMR (DMSO-d6, 400 MHz): δ 13.37 (s, 1H), 8.47 (s 1H), 8.16 (s, 1H), 8.03 (s, 1H), 7.96 (s, 1H), 7.63 (s, 2H), 6.54-6.52 (d, J = 6.4 Hz, 1H), 5.95-5.94 (d, J = 6.4 Hz, 1H), 4.36 (s, 2H), 3.58 (s, 3H), 3.17- 3.12 (m, 4H), 2.97-2.94 (m, 2H), 1.52-1.49 (d, J = 11.6 Hz, 2H) |
| I-93 | 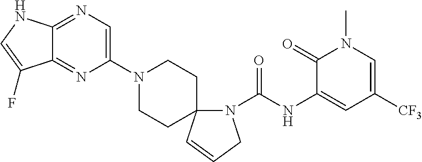 [SMILES: Cn1cc(C(F)(F)F)cc(NC(=O)N2CC=CC23CCN(c2cnc4[nH]cc(F)c4n2)CC3)c1=O] 8-(7-fluoro-5H-pyrrolo[2,3-b]pyrazin- 2-yl)-N-(1-methyl-2-oxo-5- (trifluoromethyl)-1,2-dihydropyridin-3- yl)-1,8-diazaspiro[4.5]dec-3-ene-1- carboxamide | MS (ES): m/z 492.31 [M + H]⁺ ¹H NMR (DMSO-d6, 400 MHz): δ 11.39 (s, 1H), 8.17 (s 1H), 8.06 (s, 1H), 8.00 (s, 1H), 7.60 (s, 1H), 7.57 (s, 1H), 6.62-6.60 (d, J = 5.6 Hz, 1H), 5.97-5.95 (d, J = 6.0 Hz, 1H), 4.36-4.33 (m, 4H), 3.56 (s, 3H), 3.11-3.04 (m, 2H), 2.94-2.89 (s, 2H), 1.50- 1.47 (2H) |

### Example 82: (S)-1-(4-(5H-pyrrolo[2,3-b]pyrazin-2-yl)cyclohex-3-en-1-yl)-1-methyl-3-(1-methyl-2-oxo-5-(trifluoromethyl)-1,2-dihydropyridin-3-yl)urea and (R-1-(4-(5H-pyrrolo[2,3-b]pyrazin-2-yl)cyclohex-3-en-1-yl)-1-methyl-3-(1-methyl-2-oxo-5-(trifluoromethyl)-1,2-dihydropyridin-3-yl)urea

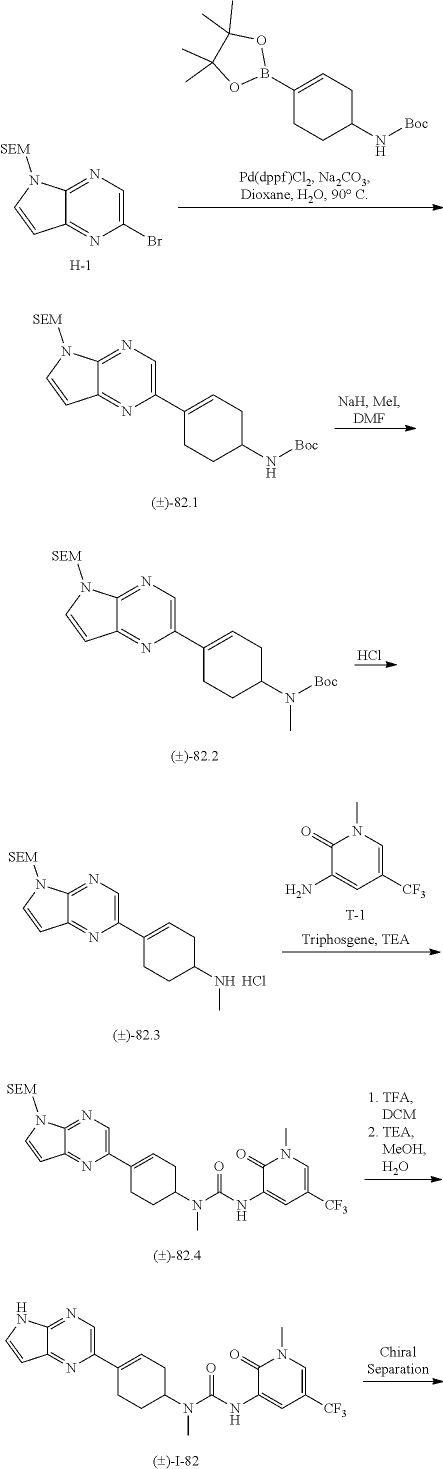

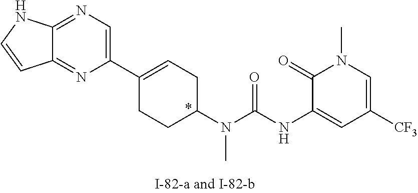 [SMILES: CN(C(=O)Nc1cc(C(F)(F)F)cn(C)c1=O)C1CC=C(c2cnc3[nH]ccc3n2)CC1]

Synthesis of compound (±)-82.1. A mixture of H-1 (1.5 g, 4.56 mmol, 1.0 equiv), 1,4-dioxane (10 mL), water (2.0 mL), tert-butyl (4-(4,4,5,5-tetramethyl-1,3,2-dioxaborolan-2-yl)cyclohex-3-en-1-yl)carbamate (1.6 g, 5.03 mmol, 1.1 equiv) and sodium carbonate (1.4 g, 13.68 mmol, 3.0 equiv) was degassed by bubbling through a stream of argon for 10 min. Dichlorobis(triphenylphosphine)palladium(II) (0.336 g, 0.456 mmol, 0.1 equiv) was added and degassed for 5 min. The reaction mixture was stirred at 90° C. for 1 h. It was cooled to room temperature, transferred into water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 24% ethyl acetate in hexane) to afford (±)-82.1 (1.2 g, 59%). MS(ES): m/z 445.26 [M+H]⁺.

Synthesis of compound (±)-82.2. To a solution of (±)-82.1 (1.2 g, 2.69 mmol, 1.0 equiv) in DMF (20 mL) at 0° C. was added sodium hydride (0.106 g, 4.04 mmol, 1.5 equiv) in portions and stirred for 20 min. To the mixture was added methyl iodide (0.33 mL, 5.38 mmol, 2.0 equiv). It was stirred at room temperature for 1 h, transferred into ice-water, stirred and extracted with ethyl acetate. The combined organic layers were washed with brine, dried over anhydrous sodium sulfate, filtered, and concentrated under reduced pressure. The residue was purified by flash column chromatography on silica gel (CombiFlash®, 17% ethyl acetate in hexane) to afford (±)-82.2 (1.3 g, 97%). MS (ES): m/z 459.27 [M+H]⁺.

Synthesis of compound (±)-82.3. To a solution of (±)-82.2 (1.3 g, 3.04 mmol, 1.0 equiv) in DCM (10 mL) was added 4 M hydrochloric acid in diethyl ether (4.0 mL) at 0° C. The mixture was stirred for 1 h and concentrated under reduced pressure. The resulting solid was used in next step without further purification (0.68 g, 49%). MS (ES): m/z 359.22 [M+H]⁺.

Synthesis of compound (±)-82.4. Compound (±)-82.4 was prepared from compound (±)-82.3 and T-1 following the procedure described in the synthesis of I-9. The product was purified by flash column chromatography on silica gel (CombiFlash®, 2% methanol in DCM). MS (ES): m/z 577.25 [M+H]⁺.

Synthesis of compound (±)-I-82. Compound (±)-I-82 was prepared from compound (±)-82.4 following the procedure described in the synthesis of I-11. The product was purified by flash column chromatography on silica gel (CombiFlash®, 50% ethyl acetate in hexane). MS (ES): m/z 447.17 [M+H]⁺.

I-82-a and I-82-b: The racemate was separated by chiral SFC (Method F) to afford first eluting fraction (I-82-a) and second eluting fraction (I-82-b). (*Absolute configuration was not determined.)

I-82-a: MS(ES): m/z: 447.36 [M+H]⁺, ¹H-NMR (400 MHz, DMSO-d₆): δ 11.96 (s, 1H), 8.49 (s, 1H), 8.18 (s, 1H), 8.02 (s, 1H), 7.93 (s, 1H), 7.81 (s, 1H), 6.64 (s, 1H), 6.58 (s, 1H), 4.39-4.31 (m, 1H), 3.59 (s, 3H), 2.92 (s, 3H), 2.60-2.57 (m, 2H), 2.32-2.28 (m, 2H), 1.93-1.88 (m, 2H).

I-82-b: MS(ES): m/z 447.35 [M+H]⁺, ¹H-NMR (400 MHz, DMSO-d₆): δ 11.96 (s, 1H), 8.50 (s, 1H), 8.18 (d, J=2.4 Hz, 1H), 8.03 (s, 1H), 7.93 (s, 1H), 7.82-7.81 (t, J=3.2 Hz, 1H), 6.64 (s, 1H), 6.58-6.57 (m, 1H), 4.39-4.31 (m, 1H), 3.59 (s, 3H), 2.92 (s, 3H), 2.60-2.57 (m, 2H), 2.32-2.28 (m, 2H), 1.93-1.88 (m, 2H).

The following compounds were prepared following the procedure of 1-82:

| Cmpd. | | |
|---|---|---|
| No. | Structure and Name | Analytical Data |
| I-160-a^{a} and I- 160-b^{b} | 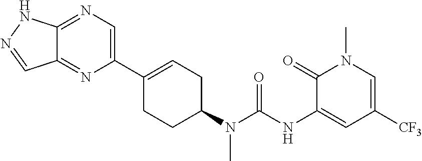 [SMILES: CN(C(=O)Nc1cc(C(F)(F)F)cn(C)c1=O)[C@@H]1CC=C(c2cnc3[nH]ncc3n2)CC1] | MS (ES): m/z 448.38 [M + H]⁺ ¹H NMR (DMSO-d₆, 400 MHz): δ 14.01 (s, 1H), 8.94 (s, 1H), 8.39 (s, 1H), 8.18 (s, 1H), 8.04 (s, 1H), 7.94 (s, 1H), 6.83 (bs, 1H), 4.39-4.37 (m, 1H), 3.59 (s, 3H), 2.93 (s, 3H), 2.64- 2.62 (m, 2H), 2.37-2.32 (m, 2H), 1.92-1.88 (m, 2H) |
| | 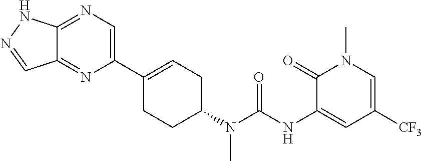 [SMILES: CN(C(=O)Nc1cc(C(F)(F)F)cn(C)c1=O)[C@H]1CC=C(c2cnc3[nH]ncc3n2)CC1] | |
| | (S)- or (R)-1-(4-(1H-pyrazolo[3,4-b]pyrain- | |
| | 5-yl)cyclohex-3-en-1-yl)-1-methyl-3-(2- | |
| | methyl-2-oxo-5-(trifluoromethyl)-1,2- | |
| | dihydropyridin-3-yl)urea | |

^{a}Separated by chiral SFC (Method D); first eluting isomer;
^{b}Separated by chiral SFC (Method D); second eluting isomer.

### Example 84: 1-((3S,4S)-3-fluoro-1-(3-methoxy-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-((3R,4R)-3-fluoro-1-(3-methoxy-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

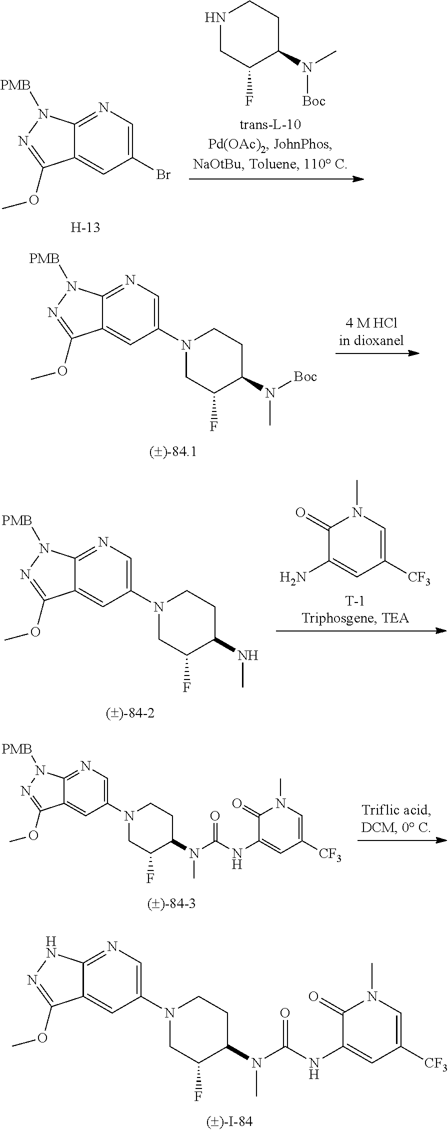

Synthesis of compound (±)-84.1. A mixture of H-13 (0.50 g, 1.43 mmol, 1.0 equiv), trans-L-10 (0.544 g, 2.33 mmol, 1.6 equiv) and sodium tert-butoxide (0.274 g, 2.86 mmol, 2.0 equiv) in toluene (2.0 mL) was bubbled through with a stream of argon for 15 min. To the mixture were added (2-biphenyl)di-tert-butylphosphine (0.042 g, 0.143 mmol, 0.1 equiv) and palladium(II) acetate (0.032 g, 0.143 mmol, 0.1 equiv). It was degassed for 10 min. The reaction mixture was stirred at 110° C. for 2 h in a microwave reactor. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford (±)-84.1 (0.390 g, 55%). MS(ES): m/z 500.28 [M+H]⁺.

Synthesis of compound (±)-84.2. To a solution of (±)-84.1 (0.39 g, 0.78 mmol, 1.0 equiv) in DCM (0.39 mL) was added 4.0 M hydrochloric acid in dioxane (0.39 mL). The reaction mixture was stirred at room temperature for 3 h. It was concentrated under reduced pressure to afford (±)-84.2 (0.39 g, 100%). MS(ES): m/z 400.21 [M+H]⁺.

Synthesis compound (±)-84.3. Compound (±)-84.3 was prepared from compound (±)-84.2 and T-1 following the procedure described in the synthesis of I-9. The product was purified by flash column chromatography on silica gel (2.5% methanol in DCM). MS(ES): m/z: 618.24 [M+H]⁺.

Synthesis of compound (±)-84. To a solution of (±)-84.3 (0.200 g, 0.32 mmol, 1.0 equiv) in DCM (2.0 mL) at 0° C. was added trifluoromethanesulfonic acid (1.0 mL). The reaction mixture was stirred at room temperature for 35 min. The reaction mixture diluted with DCM (5.0 mL), basified with saturated sodium bicarbonate solution and extracted with DCM. The combined organic layers were washed with brine, dried over anhydrous sodium sulfate, filtered, and concentrated under reduced pressure. The residue was purified by flash column chromatography on silica gel (CombiFlash®, 3.2% methanol in DCM) to afford (±)-84 (0.10 g, 62.5%). MS(ES): m/z 498.18 [M+H]⁺.

I-84-a and 1-84-b: The racemate was separated by chiral HPLC (Method E) to afford first eluting fraction (I-84-a) and second eluting fraction (I-84-b).

I-84-a: MS(ES): m/z: 498.35 [M+H], ¹H-NMR (400 MHz, DMSO-d₆): δ 12.31 (s, 1H), 8.45 (s, 1H), 8.15 (s, 1H), 8.04 (s, 1H), 7.98 (s, 1H), 7.56 (s, 1H), 4.97-4.83 (m, 1H), 4.36-4.22 (m, 1H), 3.98 (s, 3H), 3.59 (s, 3H), 3.56-3.51 (m, 2H), 2.99 (s, 3H), 2.84-2.78 (m, 2H), 1.99-1.96 (m, 1H), 1.82-1.76 (m, 1H).

I-84-b: MS(ES): m/z: 498.18 [M+H]⁺, ¹H-NMR (400 MHz, DMSO-d₆): δ 12.31 (s, 1H), 8.44 (s, 1H), 8.14 (s, 1H), 8.04 (s, 1H), 7.98 (s, 1H), 7.55 (s, 1H), 4.97-4.83 (s, 1H), 4.36-4.21 (i, 1H), 3.97 (s, 3H), 3.58 (s, 3H), 3.56-3.52 (m, 2H), 2.98 (s, 3H), 2.84-2.78 (m, 2H), 1.98-1.96 (NH, 1H), 1.82-1.76 (7, 1H).

The following compounds were prepared following the procedure of I-84:

| Cmpd. | | |
|---|---|---|
| No. | Structure and Name | Analytical Data |
| I-87-aª and I- 87-b^{b} | 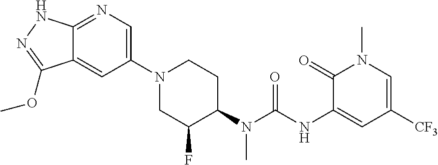 [SMILES: COc1n[nH]c2ncc(N3CC[C@@H](N(C)C(=O)Nc4cc(C(F)(F)F)cn(C)c4=O)[C@@H](F)C3)cc12] 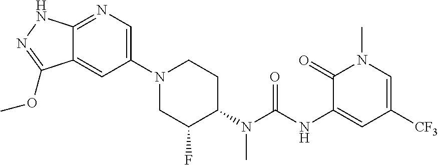 [SMILES: COc1n[nH]c2ncc(N3CC[C@H](N(C)C(=O)Nc4cc(C(F)(F)F)cn(C)c4=O)[C@H](F)C3)cc12] 1-((3S,4R)-3-fluoro-1-(3-methoxy-1H-pyrazolo[3,4- b]pyridin-5-yl)piperidin-4-yl)-1-methyl-3-(1- methyl-2-oxo-5-(trifluoromethyl)-1,2- dihydropyridin-3-yl)urea; 1-((3R,4S)-3-fluoro-1-(3-methoxy-1H-pyrazolo[3,4- b]pyridin-5-yl)piperidin-4-yl)-1-methyl-3-(1- methyl-2-oxo-5-(trifluoromethyl)-1,2- dihydropyridin-3-yl)urea | MS (ES): m/z 498.49 [M + H]⁺ 1H-NMR (400 MHz, DMSO- d₆): δ 12.27 (s, 1H), 8.41-8.40 (d, J = 2.4 Hz, 1H), 8.18-8.17 (d, J = 2.4 Hz, 1H), 8.05 (s, 1H), 7.98 (s, 1H), 7.49-7.48 (d, J = 2.4 Hz, 1H), 4.97-4.85 (d, J_{HF} = 50.4 Hz, 1H), 4.45- 4.34 (m, 1H), 3.97 (s, 3H), 3.87-3.71 (m, 2H), 3.59 (s, 3H), 3.07 (s, 3H), 3.01-2.89 (m, 1H), 2.40-2.29 (m, 2H), 1.67-1.64 (m, 1H) |

^{a}Separated by chiral HPLC (Method C); first eluting isomer;
^{b}Separated by chiral HPLC (Method C); second eluting isomer.

### Example 85: 1-(1-(5H-pyrrolo[2,3-b]pyrazin-2-yl)piperidin-4-yl)-N-(1-methyl-2-oxo-5-(trifluoromethyl)-1,2-dihydropyridin-3-yl)cyclopropane-1-carboxamide

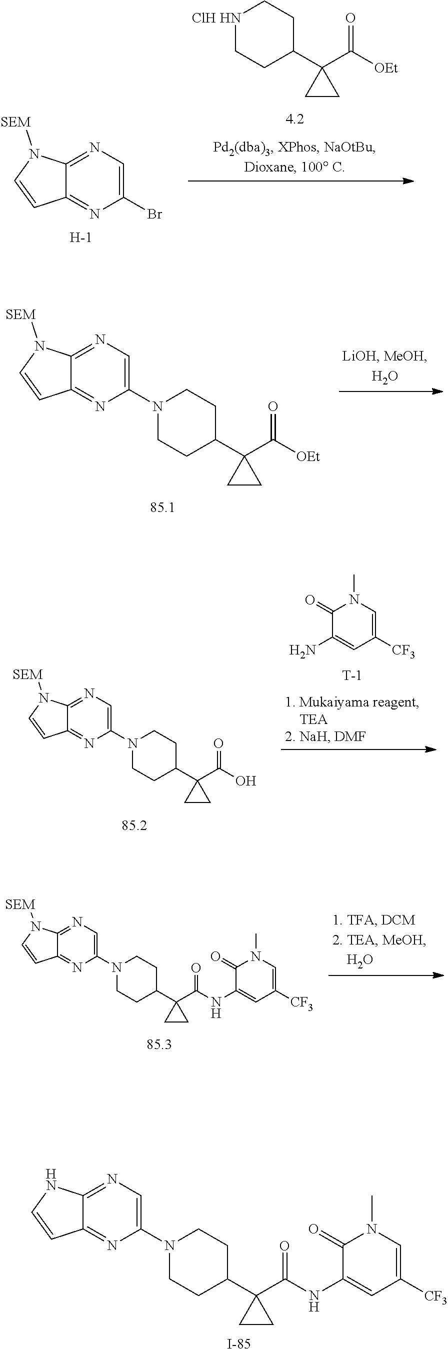

Synthesis of compound 85.1. Compound 85.1 was prepared from compound H-1 and 4.2 following the procedure described in the synthesis of 11.1. The product was purified by flash column chromatography on silica gel (CombiFlash®, 20% ethyl acetate in hexane). MS(ES): m/z 445.26 [M+H]⁺.

Synthesis of compound 85.2. To a solution of 85.1 (0.600 g, 1.34 mmol, 1.0 equiv) in methanol (5.0 mL) was added a solution of lithium hydroxide (0.323 g, 13.48 mmol, 10 equiv) in water (2.0 mL) and the reaction mixture was stirred at 55-60° C. for 3 h. It was concentrated under reduced pressure. To the residue was added water and acidified with 1 N hydrochloric acid to adjust to pH 6-6.5. The mixture was extracted with 10% methanol in DCM. The combined organic layers were washed with brine, dried over anhydrous sodium sulfate, filtered, and concentrated under reduced pressure to afford 85.2 (0.318 g, 57%). MS (ES): m/z 417.22 [M+H]⁺.

Synthesis of compound 85.3. To a solution of 85.2 (0.318 g, 0.764 mmol, 1.0 equiv) in DCM (7.0 mL) were added triethylamine (0.44 mL, 3.053 mmol, 4.0 equiv) at 0° C. followed by addition of 2-chloro-1-methylpyridinium iodide (0.0788 g, 0.305 mmol, 0.4 equiv). The reaction mixture was stirred for 3.0 h. To another flask with a solution of T-1 (0.146 g, 0.764 mmol, 1.0 equiv) in DMF (5.0 mL) were added sodium hydride (0.121 g, 3.05 mol, 4.0 equiv) at 0° C. and stirred for 2 h. It was transferred to the reaction mixture of 85.2 and stirred at room temperature for 16 h. It was transferred into water and extracted with ethyl acetate. The combined organic layers were washed with brine, dried over anhydrous sodium sulfate, filtered, and concentrated under reduced pressure to afford 85.3 (105 mg, 21%). MS(ES): m/z 591.26 [M+H]⁺.

Synthesis of I-85. Compound I-85 was prepared from compound 85.3 following the procedure described in the synthesis of I-11. The product was purified by flash column chromatography on silica gel (CombiFlash®, 5.0% methanol in DCM). MS(ES): m/z 461 [M+H]⁺, ¹H NMR (DMSO-d₆, 400 MHz): δ 11.51 (s, 1H), 8.75 (s, 1H), 8.27 (s, 1H), 8.13 (s, 1H), 8.00 (s, 1H), 7.55 (s, 1H), 6.30 (s, 1H), 4.32-4.29 (m, 1H), 3.57 (s, 3H), 2.77-2.71 (m, 2H), 1.90-1.84 (m, 2H), 1.77-1.74 (m, 2H), 1.40-1.37 (m, 2H), 0.96 (s, 2H), 0.86 (s, 2H).

### Example 91: 1-(1-(imidazo[1,5-a]pyrimidin-3-yl)piperidin-4-yl)-1-methyl-3-(1-methyl-2-oxo-5-(trifluoromethyl)-1,2-dihydropyridin-3-yl)urea

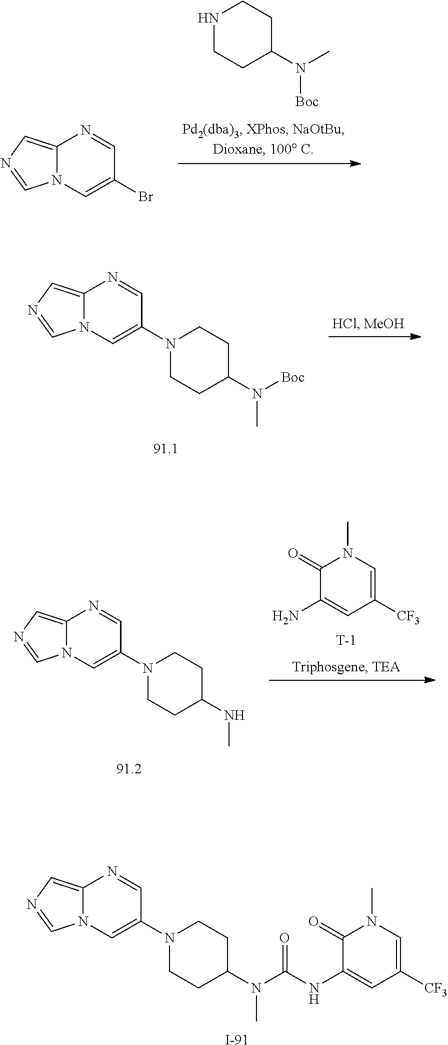

Synthesis of compound 91.1. Compound 91.1 was prepared from 3-bromoimidazo[1,5-a]pyrimidine and tert-butyl methyl(piperidin-4-yl)carbamate following the procedure described in the synthesis of 11.1. The product was purified by flash column chromatography on silica gel (CombiFlash®, 25% ethyl acetate in hexane). MS(ES): m/z 332.20 [M+H]⁺.

Synthesis of compound 91.2. To a solution of 91.1 (0.090 g, 0.27 mmol, 1.0 equiv) in DCM (1.0 mL) was added 4.0 M hydrochloric acid in dioxane (0.5 mL). The reaction mixture was stirred at room temperature for 2 h. It was concentrated under reduced pressure. The residue was purified by trituration with diethyl ether to afford 91.2 (0.090 g, 100%). MS(ES): m/z 232.15 [M+H]⁺.

Synthesis compound I-91. Compound I-91 was prepared from compound 91.2 and T-1 following the procedure described in the synthesis of 1-9. The product was purified by flash column chromatography on silica gel (2.5% methanol in DCM). MS(ES): m/z 450.36 [M+H]*, ¹H NMR (DMSO-d₆, 400 MHz): δ 8.31 (s, 1H), 8.23 (s, 1H), 8.18 (s, 1H), 8.12-8.11 (d, J=4.4 Hz, 1H), 8.03 (s, 1H), 7.93 (s, 1H), 4.39-4.28 (m, 1H), 3.75-3.72 (m, 2H), 3.59 (s, 3H), 3.00-2.97 (m, 2H), 2.94 (s, 3H), 2.08-1.99 (m, 2H), 1.75-1.73 (m, 2H).

### Example 92: 1-methyl-3-(1-methyl-2-oxo-5-(trifluoromethyl)-1,2-dihydropyridin-3-yl)-1-(1-(6-(methylamino)pyrazolo[1,5-a]pyrazin-3-yl)piperidin-4-yl)urea

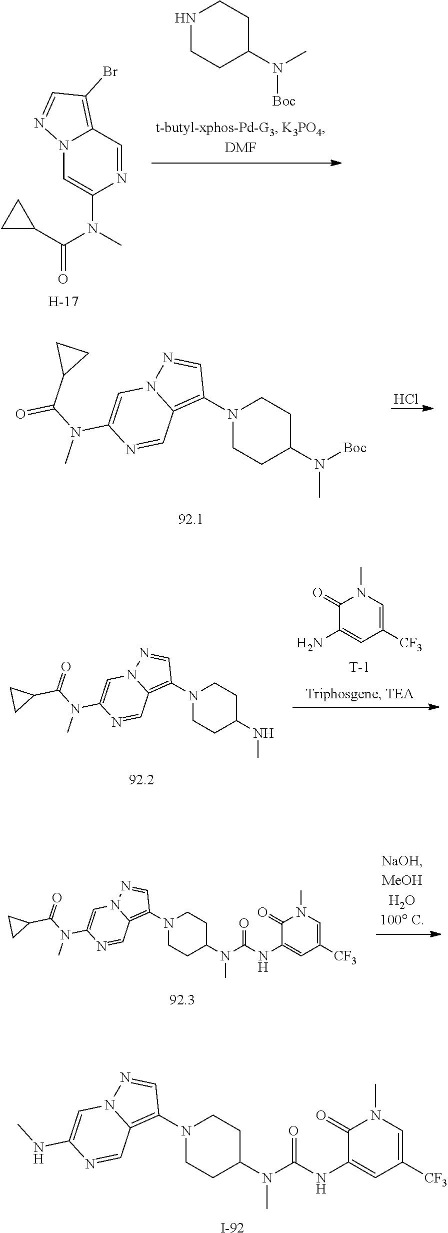

Synthesis of compound 92.1. A solution of H-17 (0.280 g, 0.94 mmol, 1.0 equiv), tert-butyl methyl(piperidin-4-yl)carbamate (0.223 g, 1.04 mmol, 1.1 equiv) and potassium phosphate (0.597 g, 2.82 mmol, 3.0 equiv) in DMF (3.0 mL) was degassed by bubbling through a stream of argon for 10 min. [(2-Di-tert-butylphosphino-2′,4′,6′-triisopropyl-1,1′-biphenyl)-2-(2′-amino-1,1′-biphenyl)]palladium(II) methanesulfonate (0.223 g, 0.282 mmol, 0.3 equiv) was added and degassed for 5 min. The reaction mixture was stirred at room temperature for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5.0% methanol in DCM) to afford 92.1 (0.04 g, 10%). MS(ES): m/z 429.25 [M+H]⁺.

Synthesis of compound 92.2. To a solution of 92.1 (0.04 g, 0.09 mmol, 1.0 equiv) in 1,4-dioxane (0.8 mL) was added 4 M hydrochloric acid in 1,4-dioxane (0.3 mL) dropwise at 0° C. The reaction mixture was stirred at room temperature for 60 min. It was concentrated under reduced pressure to afford 92.2 (0.028 g, 91%). MS(ES): m/z 329.20 [M+H]⁺.

Synthesis compound 92.3. Compound 92.3 was prepared from compound 92.2 and T-1 following the procedure described in the synthesis of I-9. The product was purified by flash column chromatography on silica gel (2.5% methanol in DCM). MS(ES): m/z: 547.23 [M+H]⁺.

Synthesis of I-92. To a solution of 92.3 (0.030 g, 0.054 mmol, 1.0 equiv) in methanol (5 mL) was added solution of sodium hydroxide (0.088 g, 2.19 mmol, 40 equiv) in water (2 mL) The reaction mixture was stirred at 80° C. for 3 h. It concentrated under reduced pressure. The residue was transferred into water and extracted with DCM. The combined organic layers were washed with brine, dried over anhydrous sodium sulfate, filtered, and concentrated under reduced pressure. The residue was purified by trituration with methanol to afford I-92 (0.015 g, 57%). MS(ES): m/z: 479.42 [M+H]⁺, ¹H NMR (DMSO-d₆, 400 MHz): 8.69 (s, 1H), 8.41 (s, 1H), 7.94 (s, 1H), 7.54 (s, 1H), 7.46 (s, 1H), 7.45 (s, 1H), 5.95 (s, 1H), 4.47-4.42 (m, 1H), 3.67 (s, 3H), 3.57-3.51 (m, 1H), 3.05 (s, 3H), 3.04-2.91 (m, 2H), 2.87 (s, 3H), 2.35-2.23 (m, 1H), 2.12-2.03 (m, 2H), 1.86-1.84 (m, 2H).

### Example 94: 1-(1-(3-methoxy-1H-pyrrolo[2,3-b]pyridin-5-yl)piperidin-4-yl)-1-methyl-3-(1-methyl-2-oxo-5-(trifluoromethyl)-1,2-dihydropyridin-3-yl)urea

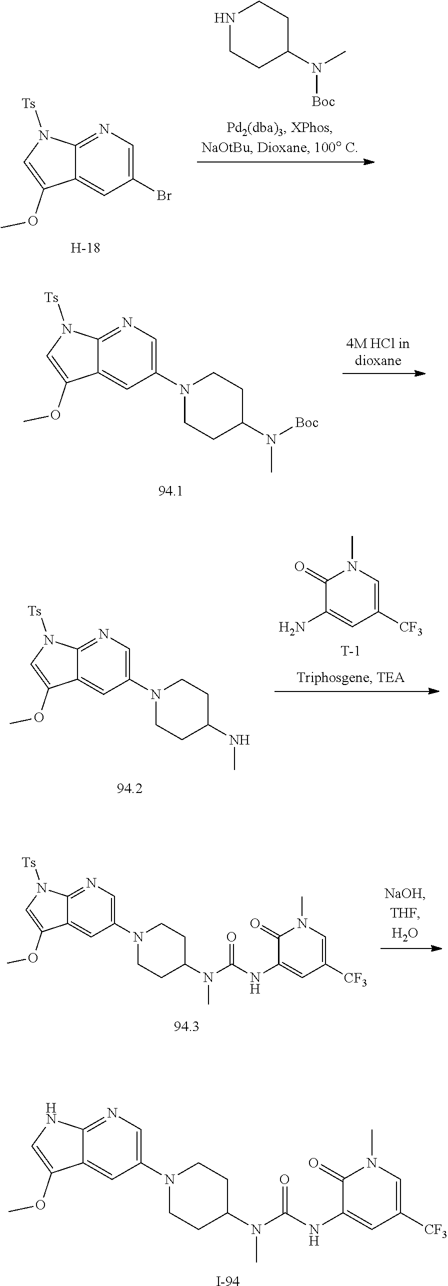

Synthesis of compound 94.1. Compound 94.1 was prepared from H-18 and tert-butyl methyl(piperidin-4-yl)carbamate following the procedure described in the synthesis of 11.1. The product was purified by flash column chromatography on silica gel (CombiFlash®, 40% ethyl acetate in hexane). MS(ES): m/z: 515.22 [M+H]⁺.

Synthesis of compound 94.2. To a solution of 94.1 (0.110 g, 0.21 mmol, 1.0 equiv) in DCM (0.7 mL) was added 4 M hydrochloric acid in 1,4-dioxane (0.7 mL) dropwise at 0° C. The reaction mixture was stirred at room temperature for 30 min. It was concentrated under reduced pressure to afford 94.2 (0.085 g, 97%). MS(ES): m/z 415.17 [M+H]⁺.

Synthesis of compound 94.3. Compound 94.3 was prepared from compound 94.2 and T-1 following the procedure described in the synthesis of I-9. The product was purified by flash column chromatography on silica gel (3.0% methanol in DCM). MS(ES): m/z 633.20 [M+H]⁺.

Synthesis of 1-94. To a solution of 94.3 (0.075 g, 0.12 mmol, 1.0 equiv) in THE (1.0 mL) and water (0.5 mL) was added sodium hydroxide (0.024 g, 0.60 mmol, 5.0 equiv) in portions. The reaction mixture was stirred 80° C. for 6 h. It was poured over water and extracted with 2.0% methanol in DCM. The combined organic layers were washed with brine, dried over anhydrous sodium sulfate, filtered, and concentrated under reduced pressure. The residue was purified by flash column chromatography on silica gel (CombiFlash®, 3.5% methanol in DCM) to afford 1-94 (0.016 g, 29%). MS(ES): m/z 489.22 [M+H]⁺, ¹H NMR (DMSO-d₆, 400 MHz): δ 10.72 (s, 1H), 8.17 (s 1H), 8.07 (s, 1H), 8.03 (s, 1H), 7.91 (s, 1H), 7.39 (s, 1H), 6.94 (s, 1H), 4.19-4.12 (m, 1H), 3.77 (s, 3H), 3.58 (s, 3H), 3.57-3.54 (m, 2H), 2.90 (s, 3H), 2.77-2.76 (m, 2H), 1.92-1.90 (m, 2H), 1.68-1.66 (m, 2H).

### Example 96: (R)-1-(3,3-difluoro-1-(2-methyl-3-oxo-2,3-dihydro-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and (S)-1-(3,3-difluoro-1-(2-methyl-3-oxo-2,3-dihydro-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

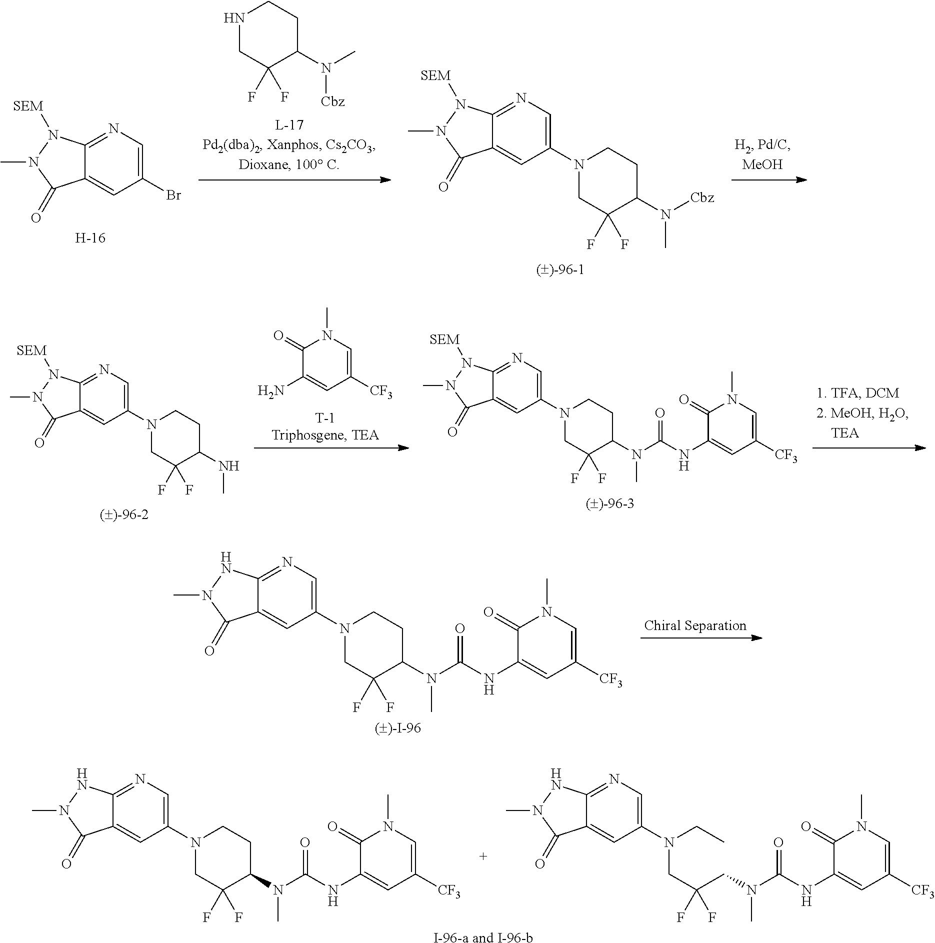

Synthesis of compound (±)-96.1. A mixture of H-16 (0.50 g, 1.40 mmol, 1.0 equiv), L-17 (0.518 g, 1.82 mmol, 1.2 equiv) and cesium carbonate (1.4 g, 4.20 mmol, 3.0 equiv) in 1,4-dioxane (6.0 mL) was degassed by bubbling through a stream of argon for 10 min. Tris(dibenzylideneacetone)dipalladium(O) (0.128 g, 0.14 mmol, 0.1 equiv) and 4,5-bis(diphenyl phosphino)-9,9-dimethyl-9H-xanthene (0.142 g, 0.14 mmol, 0.1 equiv) was added and degassed for 5 min. The reaction mixture was stirred at 100° C. for 1 h in a microwave reactor. It was cooled to room temperature, transferred into water, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford (±)-96.1 (0.320 g, 41%). MS(ES): m/z 562.26 [M+H]⁺.

Synthesis of compound (±)-96.2. A mixture of compound (±)-96.1 (0.32 g, 0.57 mmol, 1.0 equiv), methanol (10 mL) and 10% palladium on carbon (0.31 g) was stirred under hydrogen (1 atm) for 1 h. The reaction mixture was filtered through a pad of Celite® and rinsed with methanol. The filtrate was concentrated under reduced pressure to afford (±)-96.2 (0.19 g, 70%), MS(ES): m/z 428.22 [M+H]⁺.

Synthesis compound (±)-96.3. Compound (±)-96.3 was prepared from compound (±)-96.2 and T-1 following the procedure described in the synthesis of I-9. The product was purified by flash column chromatography on silica gel (2.5% methanol in DCM). MS(ES): m/z 646.25 [M+H]⁺.

Synthesis of compound (±)-96. Compound (±)-96 was prepared from compound (f)-96.3 following the procedure described in the synthesis of I-11. The product was purified by flash column chromatography on silica gel (CombiFlash®, 2.5% methanol in DCM). MS(ES): m/z 516.17 [M+H]⁺.

I-96-a and I-96-b: The racemate was separated by chiral SFC (Method D) to afford first eluting fraction (I-96-a) and second eluting fraction (I-96-b).

I-96-a: MS(ES): m/z: 516.12 [M+H]⁺, ¹H-NMR (400 MHz, DMSO-d₆): δ 10.55 (s, 1H), 8.47 (s, 1H), 8.15 (s, 1H), 8.06 (s, 2H), 7.67 (s, 1H), 5.75 (s, 1H), 4.83-4.74 (m, 1H), 4.02-3.95 (m, 1H), 3.80-3.77 (m, 1H), 3.60 (s, 3H), 3.36 (s, 3H), 3.06-3.02 (m, 1H), 2.97 (s, 3H), 2.27-2.23 (m 1H), 1.81-1.89 (m, 1H).

I-96-b: MS(ES): m/z 516.20, ¹H-NMR (400 MHz, DMSO-d₆): ¹H-NMR (400 MHz, DMSO-d₆): δ 10.64 (s, 1H), 8.45 (s, 1H), 8.15 (s, 1H), 8.06 (s, 2H), 7.66 (s, 1H), 5.75 (s, 1H), 4.83-4.74 (m, 1H), 4.02-3.95 (m, 1H), 3.80-3.77 (m, 1H), 3.60 (s, 3H), 3.36 (s, 3H), 3.06-3.02 (m, 2H), 2.97 (s, 3H), 2.26-2.22 (m 1H), 1.81-1.89 (m, 1H).

### Example 97: 1-((3S,4R)-3-fluoro-1-(3-(methylamino)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-((3R,4S)-3-fluoro-1-(3-(methylamino)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

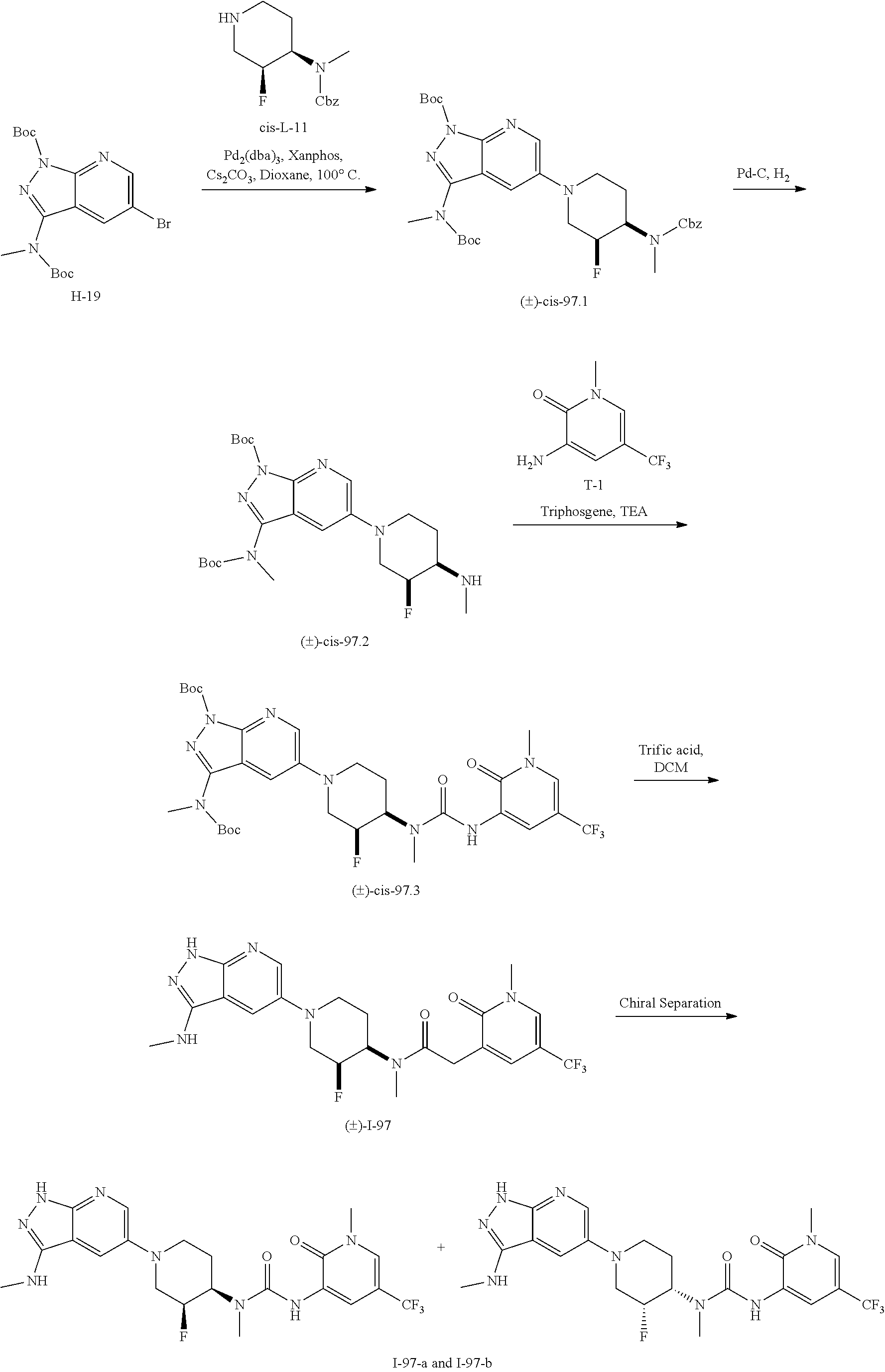

Synthesis of compound (±)-cis-97.1. Compound (±)-cis-97.1 was prepared from H-16 and L-17 following the procedure described in the synthesis of (±)-96.1. The product was purified by flash column chromatography on silica gel (CombiFlash®, 23% ethyl acetate in hexane). MS(ES): m/z 613.31 [M+H]⁺.

Synthesis of compound (±)-cis-97.2. A mixture of (±)-cis-97.1 (0.208 g, 0.34 mmol, 1.0 equiv), methanol (7.0 mL) and 10% palladium on charcoal (0.03 g) was stirred under hydrogen atmosphere at room temperature for 6 h. It was filtered through a pad of Celite®. The filtrate was concentrated under reduced pressure to afford (±)-cis-97.2 (0.110 g, 68%). MS(ES): m/z 479.27 [M+H]⁺.

Synthesis of compound (±)-cis-97.3. Compound (±)-cis-97.3 was prepared from compound (±)-cis-97.2 and T-1 following the procedure described in the synthesis of I-9. The product was purified by flash column chromatography on silica gel (CombiFlash®, 79% ethyl acetate in hexane). MS(ES): m/z 697.70 [M+H]⁺.

Synthesis of compound (±)-I-97. To a solution of compound (±)-cis-97.3 (0.07 g, 0.10 mol, 1.0 equiv) in DCM (1 mL) was added trifluoromethanesulfonic acid (0.5 mL) at 0° C. The reaction mixture was stirred at room temperature for 30 min. It was concentrated under reduced pressure. The residue was dissolved in DCM and washed with a saturate aqueous solution of sodium bicarbonate. The organic layer was washed with brine, dried over anhydrous sodium sulfate, filtered, and concentrated under reduced pressure. The residue was purified by flash column chromatography on silica gel (CombiFlash®, 2.3% methanol in DCM) to afford (±)-I-97 (0.035 g, 70%). MS(ES): m/z 497.47 [M+H]⁺.

I-97-a and I-97-b: The racemate was separated by chiral SFC (Method D) to afford first eluting fraction (I-97-a) and second eluting fraction (I-97-b).

I-97-a: MS(ES): m/z: 497.17 [M+H]⁺, ¹H-NMR (400 MHz, DMSO-d₆): δ 11.70 (s, 1H), 8.26 (s, 1H), 8.18 (s, 1H), 8.06 (s, 1H), 7.99 (s, 1H), 7.65 (s, 1H), 5.97 (m, 1H), 4.99 (m, 1H), 4.86 (m, 1H), 4.46-4.44 (m, 1H), 4.37-4.34 (m, 1H), 3.72-3.69 (m, 1H), 3.55 (s, 3H), 2.96 (s, 3H), 2.93-2.88 (m, 2H), 2.83-2.82 (d, J=4.4 Hz, 3H), 1.69-1.66 (m, 1H).

I-97-b: MS(ES): m/z: 497.17 [M+H]⁺, 1H-NMR (400 MHz, DMSO-d₆): δ 11.70 (s, 1H), 8.26 (s, 1H), 8.18 (s, 1H), 8.06 (s, 1H), 7.99 (s, 1H), 7.65 (s, 1H), 5.95 (m, 1H), 4.99 (m, 1H), 4.86 (m, 1H), 4.43-4.34 (m, 2H), 3.72-3.69 (m, 1H), 3.59 (s, 3H), 2.97 (s, 3H), 2.93-2.88 (m, 2H), 2.83-2.82 (d, J=4.4 Hz, 3H), 1.75-1.70 (m, 1H).

The following compounds were prepared following the procedure of I-97:

| Cmpd. | | |
|---|---|---|
| No. | Structure and Name | Analytical Data |
| I-101-aª and I- 101-b^{b} | 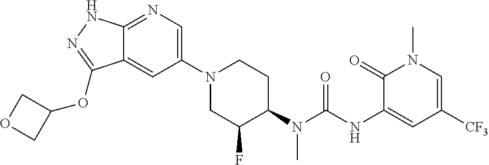 [SMILES: CN(C(=O)Nc1cc(C(F)(F)F)cn(C)c1=O)[C@@H]1CCN(c2cnc3[nH]nc(OC4COC4)c3c2)C[C@@H]1F] 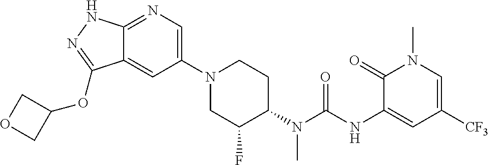 [SMILES: CN(C(=O)Nc1cc(C(F)(F)F)cn(C)c1=O)[C@H]1CCN(c2cnc3[nH]nc(OC4COC4)c3c2)C[C@H]1F] 1-((3S,4R)-3-fluoro-1-(3-(oxetan-3-yloxy)-1H- pyrazolo[3,4-b]pyridin-5-yl)piperidin-4-yl)-1- methyl-3-(1-methyl-2-oxo-5-(trifluoromethyl)-1,2- dihydropyridin-3-yl)urea; 1-((3R,4S)-3-fluoro-1-(3-(oxetan-3-yloxy)-1H- pyrazolo[3,4-b]pyridin-5-yl)piperidin-4-yl)-1- methyl-3-(1-methyl-2-oxo-5-(trifluoromethyl)-1,2- dihydropyridin-3-yl)urea | MS (ES): m/z 540.16 [M + H]⁺ ¹H-NMR (400 MHz, DMSO- d₆): δ 11.61 (s, 1H), 8.44 (s, 1H), 8.18 (s, 1H), 8.06 (s, 1H), 7.99 (s, 1H), 7.26 (s, 1H), 5.76 (m, 1H), 5.54 (m, 1H), 5.31-5.28 (m, 1H), 4.98- 4.85 (d, J_{HF} = 50.8 Hz, 1H), 4.69-4.64 (m, 1H), 4.45-4.34 (m, 1H), 3.88-3.85 (m, 1H), 3.79-3,70 (m, 2H), 3.60 (s, 3H), 3.02 (s, 3H), 2.94-2.82 (m, 2H), 2.39-2.33 (m, 1H), 1.67-1.65 (m, 1H) |
| I-102-a^{c} and I- 102-b^{d} | 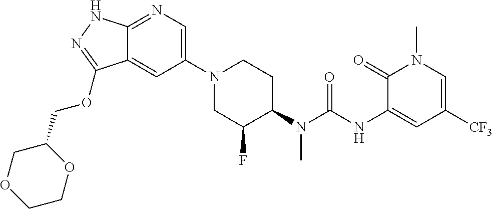 [SMILES: CN(C(=O)Nc1cc(C(F)(F)F)cn(C)c1=O)[C@@H]1CCN(c2cnc3[nH]nc(OC[C@H]4COCCO4)c3c2)C[C@@H]1F] 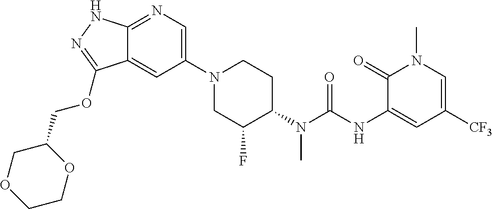 [SMILES: CN(C(=O)Nc1cc(C(F)(F)F)cn(C)c1=O)[C@H]1CCN(c2cnc3[nH]nc(OC[C@H]4COCCO4)c3c2)C[C@H]1F] 1-((3S,4R)-1-(3-(((R)-1,4-dioxan-2-yl)methoxy)- 1H-pyrazolo[3,4-b]pyridin-5-yl)-3-fluoropiperidin- 4-yl)-1-methyl-3-(1-methyl-2-oxo-5- (trifluoromethyl)-1,2-dihydropyridin-3-yl)urea; 1-((3R,4S)-1-(3-(((R)-1,4-dioxan-2-yl)methoxy)- 1H-pyrazolo[3,4-b]pyridin-5-yl)-3-fluoropiperidin- 4-yl)-1-methyl-3-(1-methyl-2-oxo-5- (trifluoromethyl)-1,2-dihydropyridin-3-yl)urea | MS (ES): m/z 584.18 [M + H]⁺ ¹H-NMR (400 MHz, DMSO- d₆): δ 12.33 (s, 1H), 8.43 (s, 1H), 8.19 (s, 1H), 8.07 (s, 1H), 8.00 (s, 1H), 7.50 (s, 1H), 4.99-4.87 (d, J_{HF} = 50 Hz, 1H), 4.51-4.32 (m, 1H), 4.28 (m, 3H), 3.96-3.91 (m, 1H), 3.88-3.78 (m, 3H), 3.70- 3.64 (m, 2H), 3.61 (s, 3H), 3.56-3.53 (m, 1H), 3.48-3.42 (m, 1H), 3.09-2.91 (m, 2H), 2.99 (s, 3H), 2.37-2.34 (m, 1H), 1.69-1.67 (m,1H) |
| I-104-a^{e} and I- 104-b^{f} | 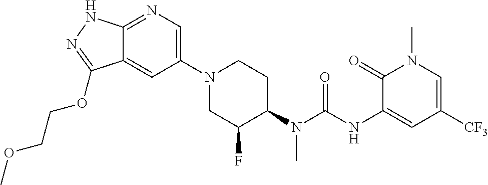 [SMILES: COCCOc1n[nH]c2ncc(N3CC[C@@H](N(C)C(=O)Nc4cc(C(F)(F)F)cn(C)c4=O)[C@@H](F)C3)cc12] 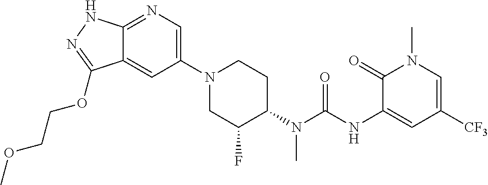 [SMILES: COCCOc1n[nH]c2ncc(N3CC[C@H](N(C)C(=O)Nc4cc(C(F)(F)F)cn(C)c4=O)[C@H](F)C3)cc12] 1-((3S,4R)-3-fluoro-1-(3-(2-methoxyethoxy)-1H- pyrazolo[3,4-b]pyridin-5-yl)piperidin-4-yl)-1- methyl-3-(1-methyl-2-oxo-5-(trifluoromethyl)-1,2- dihydropyridin-3-yl)urea; 1-((3R,4S)-3-fluoro-1-(3-(2-methoxyethoxy)-1H- pyrazolo[3,4-b]pyridin-5-yl)piperidin-4-yl)-1- methyl-3-(1-methyl-2-oxo-5-(trifluoromethyl)-1,2- dihydropyridin-3-yl)urea | MS (ES): m/z 542.41 [M + H]⁺ ¹H-NMR (400 MHz, DMSO- d₆): δ 12.29 (s, 1H), 8.41 (s, 1H), 8.18 (s, 1H), 8.06 (s, 1H), 7.99 (s, 1H), 7.47 (s, 1H), 4.98-4.85 (d, J_{HF} = 50.4 Hz, 1H), 4.42-4.32 (m, 3H), 3.82-3.73 (m, 2H), 3.60 (s, 3H), 2.93 (s, 3H), 3.11-2.93 (m, 5H), 2.50 (m, 2H), 2.36- 2.33 (m, 1H), 1.68 (m, 1H) |

^{a}Separated by chiral HPLC (Method G); first eluting isomer;
^{b}Separated by chiral HPLC (Method G); second eluting isomer;
^{c}Separated by chiral HPLC (Method C); first eluting isomer;
^{d}Separated by chiral HPLC (Method C); second eluting isomer;
^{e}Separated by chiral SFC (Method I); first eluting isomer;
^{f}Separated by chiral SFC (Method I); second eluting isomer.

### Example 98: 8-(7-cyano-5H-pyrrolo[2,3-b]pyrazin-2-yl)-N-(1-methyl-2-oxo-5-(trifluoromethyl)-1,2-dihydropyridin-3-yl)-1,8-diazaspiro[4.5]dec-3-ene-1l-carboxamide

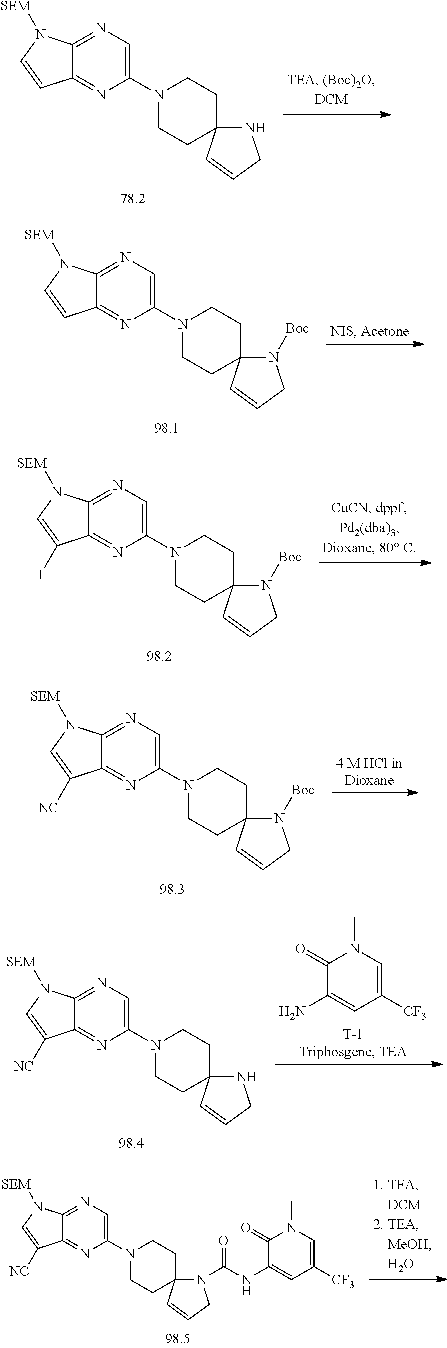

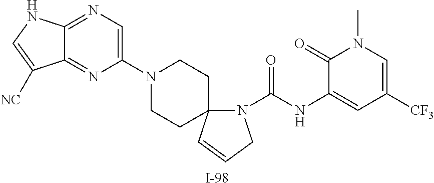

Synthesis of compound 98.1 To a solution of 78.2 (0.24 g, 0.62 mmol, 1.0 equiv) in DCM (5.0 mL) was added trimethylamine (0.1 mL, 0.12 mmol, 0.2 equiv) and di-tert-butyl dicarbonate (0.15 mL, 0.62 mmol, 1.0 equiv) dropwise at 0° C. The reaction mixture was stirred at room temperature for 2 h. It was poured over water and extracted with DCM. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford 98.1 (0.20 g, 66%). MS(ES): m/z 486.28 [M+H]⁺.

Synthesis of compound 98.2 To a solution of 98.1 (0.20 g, 0.41 mmol, 1.0 equiv) in acetone (4.0 mL) was added in portions N-iodosuccinimide (0.137 g, 0.61 mmol, 1.5 equiv) at 0° C. The reaction mixture was stirred at room temperature for 1 h. It was concentrated under reduced vacuum. To the residue was added water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford 98.2 (0.145 g, 58%). MS(ES): m/z 612.18 [M+H]⁺.

Synthesis of compound 98.3. A mixture of 98.2 (0.145 g, 0.23 mmol, 1.0 equiv) in 1,4-dioxane (5.0 mL) was degassed by bubbling through a stream of argon for 10 min followed by addition of tris(dibenzylideneacetone)dipalladium(O) (0.011 g, 0.011 mmol, 0.05 equiv), 1,1′-bis(diphenylphosphino)ferrocene (0.013 g, 0.023 mmol, 0.1 equiv) and copper(I) cyanide (0.082 g, 0.92 mmol, 4.0 equiv). The reaction mixture was stirred at 80° C. for 16 h. It was cooled to room temperature, filtered through a pad of Celite®. The filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 18% ethyl acetate in hexane) to afford 98.3 (0.085 g, 72%). MS(ES): m/z 511.28 [M+H]⁺.

Synthesis of compound 98.4. To a solution of 98.3 (0.085 g, 0.16 mmol, 1.0 equiv) in 1,4-dioxane (0.8 mL) was added 4 M hydrochloric acid in 1,4-dioxane (0.9 mL) dropwise at 0° C. The reaction mixture was stirred at room temperature for 1 h. It was concentrated under reduced pressure to afford 98.4 (0.072 g, 100%). MS(ES): m/z 411.23 [M+H]⁺.

Synthesis of compound 98.5. Compound 98.5 was prepared from compound 98.4 and T-1 following the procedure described in the synthesis of I-9. The product was purified by flash column chromatography on silica gel (3.0% methanol in DCM). MS(ES): m/z 629.26 [M+H]⁺.

Synthesis of I-98. Compound I-98 was prepared from compound 98.5 following the procedure described in the synthesis of I-11. The product was purified by flash column chromatography on silica gel (CombiFlash®, 4.0% methanol in DCM). MS(ES): m/z 499.12 [M+H]⁺, ¹H NMR (DMSO-d₆, 400 MHz): δ 11.71 (s, 1H), 8.37 (s 1H), 8.25 (s, 1H), 8.06 (s, 1H), 8.00 (s, 1H), 7.60 (s, 1H), 6.62 (s, 1H), 5.97 (s, 1H), 4.43-4.36 (m, 3H), 3.56 (s, 3H), 3.16-3.09 (2H), 2.92-2.90 (m, 2H), 2.55-2.50 (m, 2H), 1.53-1.50 (m, 1H).

### Example 100: 1-((3R,4S)-3-fluoro-1-(2-oxo-2,3-dihydro-1H-imidazo[4,5-b]pyridin-6-yl)piperidin-4-yl)-1-methyl-3-(1-methyl-2-oxo-5-(trifluoromethyl)-1,2-dihydropyridin-3-yl)urea and 1-((3S,4R)-3-fluoro-1-(2-oxo-2,3-dihydro-1H-imidazo[4,5-b]pyridin-6-yl)piperidin-4-yl)-1-methyl-3-(1-methyl-2-oxo-5-(trifluoromethyl)-1,2-dihydropyridin-3-yl)urea

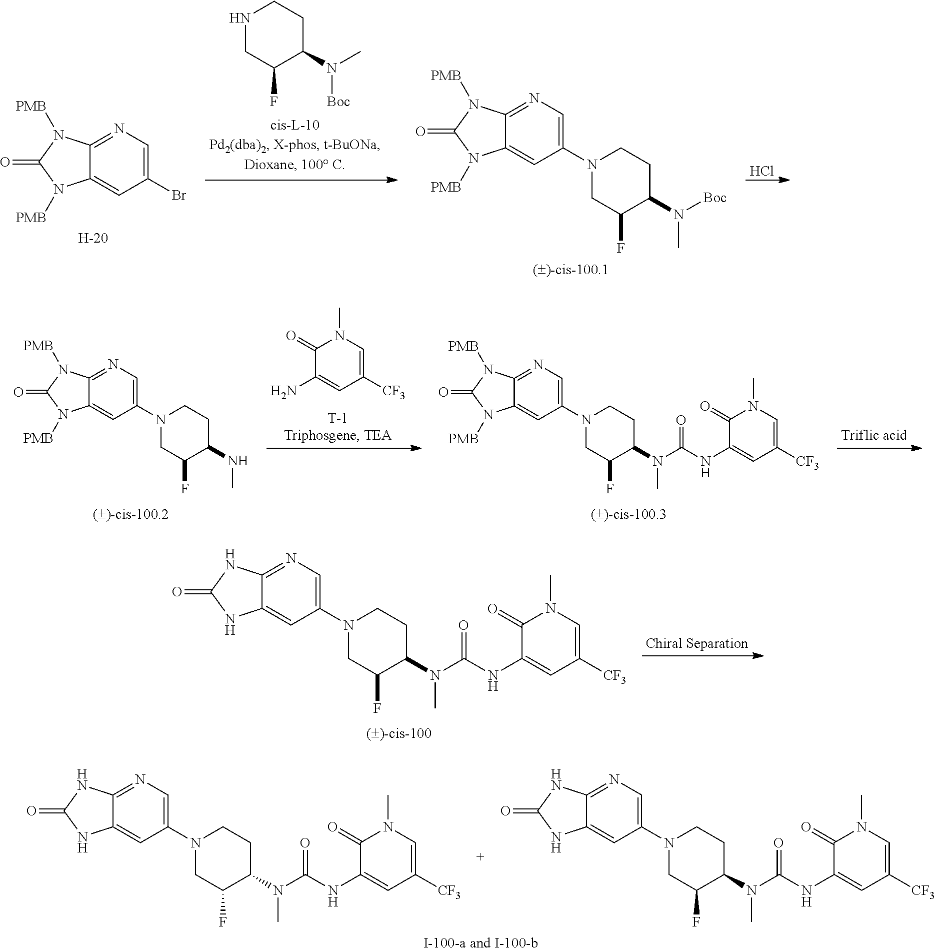

Synthesis of compound (±)-cis-100.1. Compound (±)-cis-100.1 was prepared from compound H-20 and cis-L-10 following the procedure described in the synthesis of 11.1. The product was purified by flash column chromatography on silica gel (CombiFlash®, 60% ethyl acetate in hexane). MS(ES): m/z 606.71 [M+H]⁺.

Synthesis of compound (±)-cis-100.2. To a solution of (±)-cis-100.1 (0.650 g, 1.07 mmol, 1.0 equiv) in DCM (10. mL) was added 4M hydrogen chloride in 1,4-dioxane (4.0 mL) and stirred at 0° C. for 4 h. It was concentrated under reduced pressure to afford (±)-cis-100.2 (0.5 g, 92%). MS(ES): m/z 506.59 [M+H]⁺.

Synthesis of compound (±)-cis-100.3. Compound (±)-cis-100.3 was prepared from compound (±)-cis-100.3 and T-1 following the procedure described in the synthesis of I-9. The product was purified by flash column chromatography on silica gel (CombiFlash®, 90% ethyl acetate in hexane). MS(ES): m/z 724.73 [M+H]⁺.

Synthesis of compound (±)-cis-100. To a solution of compound (±)-cis-100.3 (0.2 g, 0.27 mmol, 1.0 equiv) in DCM (4.0 mL) was added trifluoromethanesulfonic acid (0.5 mL) at 0° C. The reaction mixture was stirred at room temperature for 30 min. It was concentrated under reduced pressure. The residue was dissolved in DCM and washed with a saturated aqueous solution of sodium bicarbonate. The organic layer was washed with brine, dried over anhydrous sodium sulfate, filtered, and concentrated under reduced pressure. The residue was purified by flash column chromatography on silica gel (CombiFlash®, 2.3% methanol in DCM) to afford (±)-cis-100 (0.04 g, 20%). MS(ES): m/z 484.43 [M+H]⁺.

I-100-a and I-100-b. The racemate was separated by chiral SFC (Method H) to afford first eluting fraction (I-100-a) and second eluting fraction (I-100-b).

I-100-a: MS(ES): m/z: 484.10 [M+H]⁺, ¹H-NMR (400 MHz, DMSO-d₆): δ 10.98 (s, 1H), 10.67 (s, 1H), 8.17 (s, 1H), 8.05 (s, 1H), 7.98 (s, 1H), 7.58 (s, 1H), 6.93 (s, 1H), 4.93-4.83 (d, J_{HF}=48 Hz, 1H), 1H), 4.43-4.32 (m, 1H), 3.75-3.65 (m, 2H), 3.59 (s, 3H), 3.01 (s, 3H), 2.92-2.89 (m, 2H), 2.33-2.21 (m, 1H), 1.67-1.64 (m, 1H).

I-100-b: MS(ES): m/z: 484.43 [M+H]⁺, ¹H-NMR (400 MHz, DMSO-d₆): δ 10.98 (s, 1H), 10.67 (s, 1H), 8.17 (s, 1H), 8.05 (s, 1H), 7.98 (s, 1H), 7.58 (s, 1H), 6.93 (s, 1H), 4.95-4.83 (d, J_{HF}=48 Hz, 1H), 4.43-4.32 (m, 1H), 3.75-3.65 (m, 2H), 3.59 (s, 3H), 3.01 (s, 3H), 2.92-2.89 (m, 2H), 2.33-2.21 (m, 1H), 1.67-1.64 (m, 1H).

The following compounds were prepared following the procedure of I-100:

| Cmpd. | | |
|---|---|---|
| No. | Structure and Name | Analytical Data |
| I-108-aª and I- 108-b^{b} | 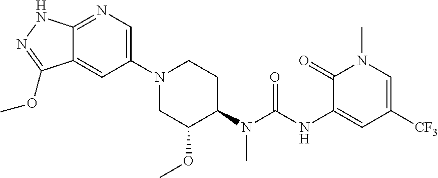 [SMILES: COc1n[nH]c2ncc(N3CC[C@@H](N(C)C(=O)Nc4cc(C(F)(F)F)cn(C)c4=O)[C@H](OC)C3)cc12] 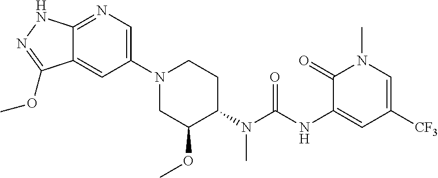 [SMILES: COc1n[nH]c2ncc(N3CC[C@H](N(C)C(=O)Nc4cc(C(F)(F)F)cn(C)c4=O)[C@@H](OC)C3)cc12] 1-((3R,4R)-3-methoxy-1-(3-methoxy-1H- pyrazolo[3,4-b]pyridin-5-yl)piperidin-4- yl)-1-methyl-3-(1-methyl-2-oxo-5- (trifluoromethyl)-1,2-dihydropyridin-3- yl)urea; 1-((3S,4S)-3-methoxy-1-(3-methoxy-1H- pyrazolo[3,4-b]pyridin-5-yl)piperidin-4- yl)-1-methyl-3-(1-methyl-2-oxo-5- (trifluoromethyl)-1,2-dihydropyridin-3- yl)urea | MS (ES): m/z 510.14 [M + H]⁺ ¹H NMR (DMSO-d₆, 400 MHz): δ 12.29 (s, 1H), 8.45 (s, 1H), 8.07 (s, 1H), 8.05 (s, 1H), 8.01 (s, 1H), 7.54 (s, 1H), 4.05-3.97 (m, 4H), 3.62-3.53 (m, 4H), 3.37-3.33 (m, 3H), 2.89 (s, 3H), 2.79-2.71 (m, 2H), 2.70-2.62 (m, 2H), 2.01-1.91 (m, 1H), 1.79-1.71 (m, 1H) |
| I-109-aª and I- 109-b^{b} | 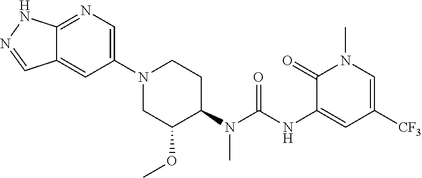 [SMILES: CO[C@@H]1CN(c2cnc3[nH]ncc3c2)CC[C@H]1N(C)C(=O)Nc1cc(C(F)(F)F)cn(C)c1=O] 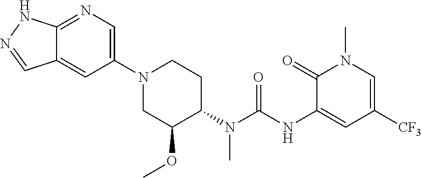 [SMILES: CO[C@H]1CN(c2cnc3[nH]ncc3c2)CC[C@@H]1N(C)C(=O)Nc1cc(C(F)(F)F)cn(C)c1=O] 1-((3R,4R)-3-methoxy-1-(1H-pyrazolo[3,4- b]pyridin-5-yl)piperidin-4-yl)-1-methyl-3- (1-methyl-2-oxo-5-(trifluoromethyl)-1,2- dihydropyridin-3-yl)urea; 1-((3S,4S)-3-methoxy-1-(1H-pyrazolo[3,4- b]pyridin-5-yl)piperidin-4-yl)-1-methyl-3- (1-methyl-2-oxo-5-(trifluoromethyl)-1,2- dihydropyridin-3-yl)urea | MS (ES): m/z 480.44 [M + H]⁺ ¹H NMR (DMSO-d₆, 400 MHz): δ 13.42 (s, 1H), 8.49 (s, 1H), 8.29 (s, 1H), 8.08 (s, 1H), 8.01 (s, 1H), 7.97 (s, 1H), 7.70 (s, 1H), 4.02-4.00 (m, 2H), 3.59-3.67 (m, 2H), 3.58 (s, 3H), 3.38 (s, 3H), 2.90 (s, 3H), 2.79-2.73 (m, 2H), 1.98-1.96 (m, 1H), 1.79-1.73 (m, 1H) |
| I-131-a^{c} and I- 131-b^{d} | 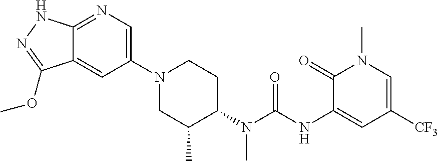 [SMILES: COc1n[nH]c2ncc(N3CC[C@H](N(C)C(=O)Nc4cc(C(F)(F)F)cn(C)c4=O)[C@H](C)C3)cc12] 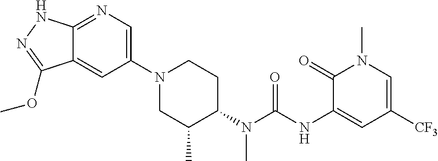 [SMILES: COc1n[nH]c2ncc(N3CC[C@H](N(C)C(=O)Nc4cc(C(F)(F)F)cn(C)c4=O)[C@H](C)C3)cc12] 1-((3R,4S)-1-(3-methoxy-1H-pyrazolo[3,4- b]pyridin-5-yl)-3-methylpiperidin-4-yl)-1- methyl-3-(1-methyl-2-oxo-5- (trifluoromethyl)-1,2-dihydropyridin-3- yl)urea; 1-((3S,4R)-1-(3-methoxy-1H-pyrazolo[3,4- b]pyridin-5-yl)-3-methylpiperidin-4-yl)-1- methyl-3-(1-methyl-2-oxo-5- (trifluoromethyl)-1,2-dihydropyridin-3- yl)urea | MS (ES): m/z 494.14 [M + H]⁺ ¹H NMR (DMSO-d₆, 400 MHz): δ 12.27 (s, 1H), 8.37 (s, 1H), 8.19 (s, 1H), 8.03 (s, 1H), 7.92 (s, 1H), 7.42 (s, 1H), 4.18-4.15 (m, 1H), 3.97 (s, 3H), 3.62-3.61 (m, 1H), 3.59 (s, 3H), 3.40- 3.33 (m, 1H), 2.97 (s, 3H), 2.88-2.86 (m, 1H), 2.78-2.72 (m, 1H), 2.35-2.24 (m, 2H), 1.64-1.61 (m, 1H), 1.13-1.11 (d, J = 6.8 Hz, 3H). |
| I-138 | 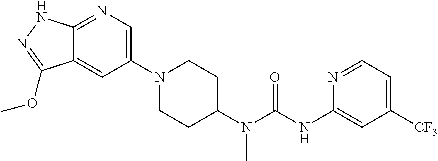 [SMILES: COc1n[nH]c2ncc(N3CCC(N(C)C(=O)Nc4cc(C(F)(F)F)ccn4)CC3)cc12] 1-(1-(3-methoxy-1H-pyrazolo[3,4- b]pyridin-5-yl)piperidin-4-yl)-1-methyl-3- (4-(trifluoromethyl)pyridin-2-yl)urea | MS (ES): m/z 450.1 [M + H]⁺ ¹H NMR (DMSO-d₆, 400 MHz): δ 12.26 (s, 1H), 9.46 (s, 1H), 8.51-8.50 (d, J = 4.8 Hz, 1H), 8.42-8.41 (d, J = 2.4 Hz, 1H), 8.18 (s, 1H), 7.46-7.45 (d, J = 2.4 Hz, 1H), 7.31-7.30 (d, J = 5.2 Hz, 1H), 4.25-4.19 (s, 1H), 3.97 (s, 3H), 3.64-3.61 (m, 2H), 2.90 (s, 3H), 2.80-2.74 (m, 2 H), 1.97-1.87 (m, 2H), 1.70-1.68 (m, 2H) |
| I-182 | 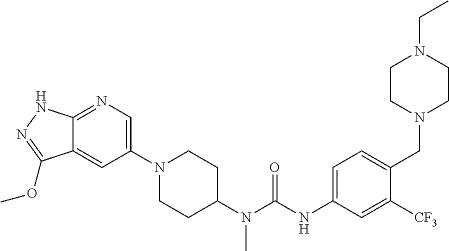 [SMILES: CCN1CCN(Cc2ccc(NC(=O)N(C)C3CCN(c4cnc5[nH]nc(OC)c5c4)CC3)cc2C(F)(F)F)CC1] 3-(4-((4-ethylpiperazin-1-yl)methyl)-3- (trifluoromethyl)phenyl)-1-(1-(3-methoxy- 1H-pyrazolo[3,4-b]pyridin-5-yl)piperidin- 4-yl)-1-methylurea | MS (ES): m/z 575.62 [M + H]⁺ ¹H-NMR (400 MHz, DMSO-d₆): δ 12.27 (s, 1H), 8.58 (s, 1H), 8.43-8.42 (d, J = 2.4 Hz, 1H), 8.15 (s, 1H), 7.91 (s, 1H), 7.76-7.74 (m, 1H), 7.58-7.56 (m, 1H), 7.47-7.46 (m, 1H), 4.22-4.16 (m, 1H), 3.98 (s, 3H), 3.66-3.63 (m, 2H), 3.52 (s, 2H), 2.87 (s, 3H), 2.80- 2.74 (m, 2H), 2.51-2.50 (m, 1H), 2.40- 2.23 (m, 8H), 1.99-1.87 (m, 2H), 1.69- 1.66 (2H), 1.02-0.98 (m, 3H) |

^{a}Separated by chiral SFC (Method E); first eluting isomer;
^{b}Separated by chiral SFC (Method E); second eluting isomer;
^{c}Separated by chiral HPLC (Method K); first eluting isomer;
^{d}Separated by chiral HPLC (Method K); second eluting isomer.

### Example 103: 1-((3R,4S)-3-fluoro-1-(1-methyl-2-oxo-2,3-dihydro-1H-imidazo[4,5-b]pyridin-6-yl)piperidin-4-yl)-1-methyl-3-(1-methyl-2-oxo-5-(trifluoromethyl)-1,2-dihydropyridin-3-yl)urea and 1-((3S,4R)-3-fluoro-1-(1-methyl-2-oxo-2,3-dihydro-1H-imidazo[4,5-b]pyridin-6-yl)piperidin-4-yl)-1-methyl-3-(1-methyl-2-oxo-5-(trifluoromethyl)-1,2-dihydropyridin-3-yl)urea

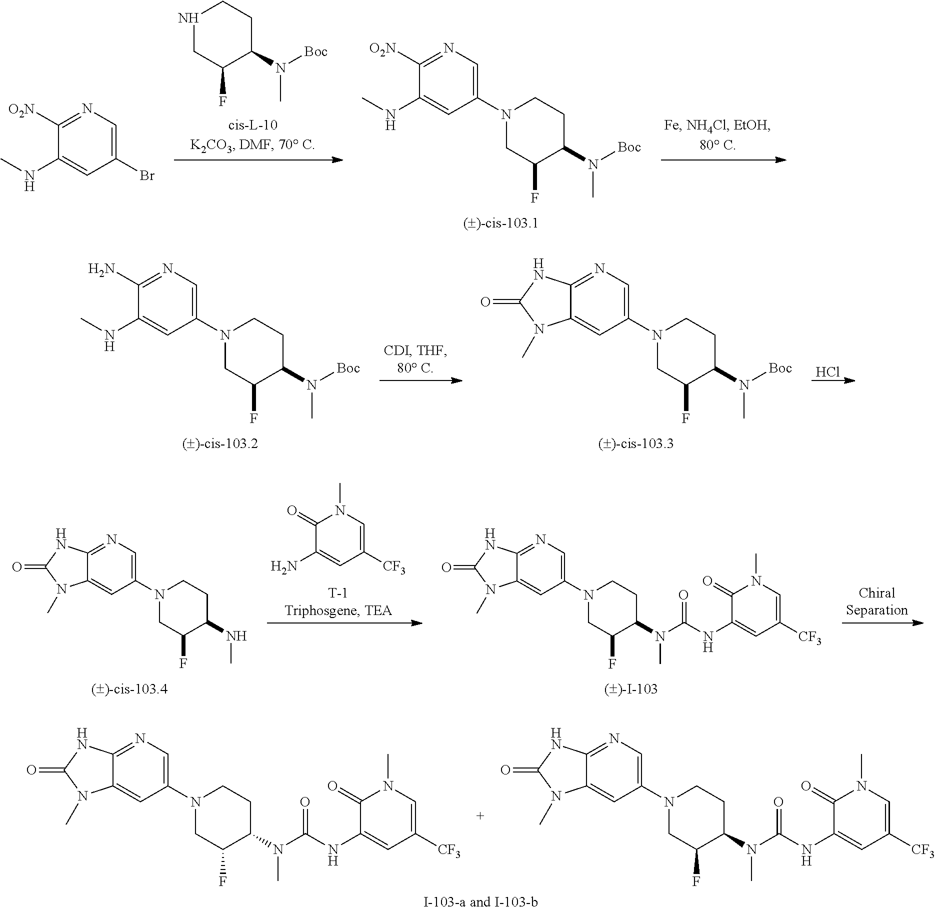

Synthesis of compound (±)-cis-103.1. A mixture of 5-fluoro-N-methyl-2-nitropyridin-3-amine (1.0 g, 5.84 mmol, 1.0 equiv), potassium carbonate (1.6 g, 11.69 mmol, 2.0 equiv) and cis-L-10 (2.0 g, 8.76 mmol, 1.5 equiv) in DMF (10 mL) was stirred at 70° C. for 3 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford (±)-cis-103.1 (1.0 g, 45%). MS(ES): m/z 384.20 [M+H]⁺.

Synthesis of compound (±)-cis-103.2. To a solution of (±)-cis-103.1 (1.0 g, 2.60 mmol, 1.0 equiv) in ethanol (20 mL) at 0° C. was added ammonium chloride (1.3 g, 26.04 mmol, 10 equiv) and iron powder (1.4 g, 26.04 mmol, 10 equiv). The reaction mixture was stirred at 80° C. for 1 h. It was cooled to rt and filtered through a pad of Celite®. The filtrate was concentrated under reduced pressure. The residue was dissolved in water and extracted with DCM. The combined organic layers were washed with brine, dried over anhydrous sodium sulfate, filtered, and concentrated under reduced pressure. The residue was purified by flash column chromatography on silica gel (CombiFlash®, 100% ethyl acetate) to afford (±)-cis-103.2 (0.80 g, 87%). MS(ES): m/z 354.22 [M+H]⁺.

Synthesis of compound (±)-cis-103.3. To a stirred solution of (±)-cis-103.2 (0.80 g, 2.25 mmol, 1.0 equiv) in THE (10 mL) was added carbonyldiimidazole (0.546 g, 3.37 mmol, 1.5 equiv). The reaction mixture was stirred at 80° C. for 1 h. It was cooled to room temperature and quenched by water and extracted with ethyl acetate. The organic layer was separated, dried over anhydrous sodium sulfate, filtered, and concentrated under reduced pressure. The residue was purified by flash column chromatography on silica gel (CombiFlash®, 30% ethyl acetate in hexane) to afford (±)-cis-103.3 (0.70 g, 82%). MS(ES): m/z: 380.20 [M+H]⁺.

Synthesis of compound (±)-cis-103.4. To a solution of (±)-cis-103.3 (0.70 g, 1.84 mmol, 1.0 equiv) in 1,4-dioxane (0.8 mL) was added 4 M hydrochloric acid in 1,4-dioxane (0.9 mL) dropwise at 0° C. The reaction mixture was stirred at room temperature for 1 h. It was concentrated under reduced pressure. The residue was used in the next step without further purification (±)-cis-103.4 (0.60 g, 100%). MS(ES): m/z 280.20 [M+H]⁺.

Synthesis compound (±)-cis-103. Compound (±)-cis-103 was prepared from compound (±)-cis-103.4 and T-1 following the procedure described in the synthesis of I-9. The product was purified by flash column chromatography on silica gel (2.5% methanol in DCM). MS(ES): m/z 498.18 [M+H]⁺.

I-103-a and I-103-b: The racemate was separated by chiral SFC (Method F) to afford first eluting fraction (I-103-a) and second eluting fraction (I-103-b).

I-103-a: MS(ES): m/z: 498.15 [M+H]⁺, ¹H-NMR (400 MHz, DMSO-d₆): δ 11.20 (s, 1H), 8.18 (s, 1H), 8.06 (s, 1H), 7.99 (s, 1H), 7.60 (s, 1H), 7.24 (s, 1H), 4.98-4.85 (d, J_{HF}=50.4 Hz, 1H), 4.46-4.34 (m, 2H), 3.89-3.75 (m, 2H), 3.60 (s, 3H), 3.27 (s, 3H), 3.02 (s, 3H), 2.97-2.88 (m, 1H), 2.33-2.30 (m, 1H), 1.68-1.65 (m, 1H).

I-103-b: MS(ES): m/z: 498.16 [M+H]⁺, ¹H-NMR (400 MHz, DMSO-d₆): δ 11.12 (s, 1H), 8.18 (s, 1H), 8.05 (s, 1H), 7.98 (s, 1H), 7.53 (s, 1H), 7.11 (s, 1H), 4.96-4.84 (d, J_{HF}=50.4 Hz, 1H), 4.44-4.33 (m, 1H), 3.79-3.68 (m, 2H), 3.59 (s, 3H), 3.23 (3H), 3.01 (s, 3H), 2.94-2.85 (m, 2H), 2.32-2.29 (m, 1H), 1.66-1.64 (m, 1H).

### Example 105: 1-methyl-1-((2S,4S)-2-methyl-1-(1H-pyrazolo[3,4-b]pyridin-5-yl)piperidin-4-yl)-3-(1-methyl-2-oxo-5-(trifluoromethyl)-1,2-dihydropyridin-3-yl)urea and 1-methyl-1-((2R,4R)-2-methyl-1-(1H-pyrazolo[3,4-b]pyridin-5-yl)piperidin-4-yl)-3-(1-methyl-2-oxo-5-(trifluoromethyl)-1,2-dihydropyridin-3-yl)urea

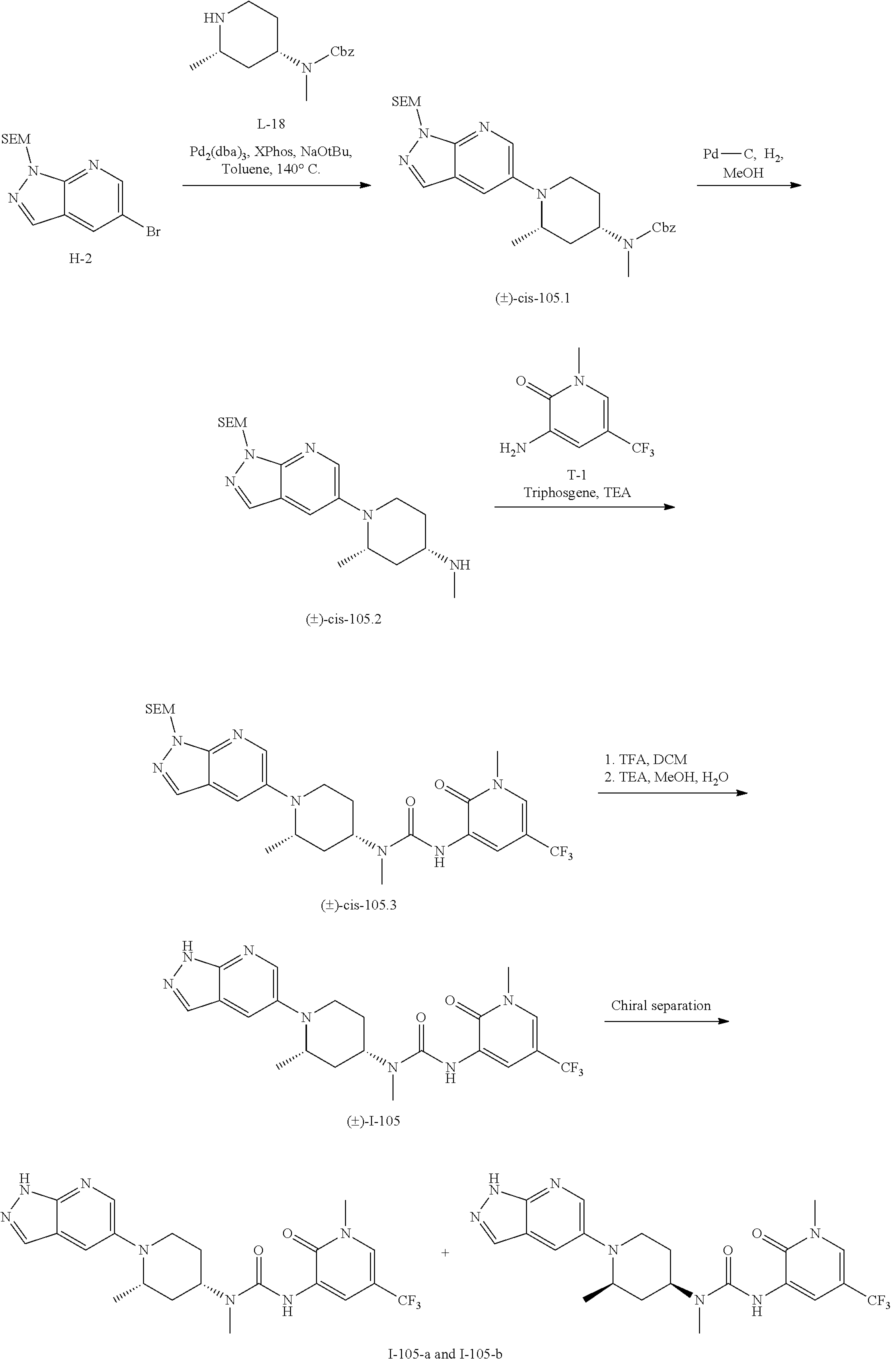

Synthesis of compound (±)-cis-105.1. A mixture of H-2 (1.7 g, 5.18 mmol, 1.0 equiv), cis-L-18 (2.1 g, 8.03 mmol, 1.55 equiv) and sodium tert-butoxide (1.24 g, 12.95 mmol, 2.5 equiv) in toluene (15 mL) was degassed by bubbling through a stream of argon for 10 min. [2-Dicyclohexylphosphino-2′,4′,6′-triisopropylbiphenyl] (0.370 g, 0.777 mmol, 0.15 equiv) and tris(dibenzylideneacetone)dipalladium(O) (0.474 g, 0.518 mmol, 0.1 equiv) were added and the reaction mixture was degassed for 15 min. The mixture was stirred at 140° C. for 1.5 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford (±)-cis-105.1 (0.370 g, 14%). MS(ES): m/z 510.28 [M+H]⁺.

Synthesis of compound (±)-cis-105.2. A mixture of compound (±)-cis-105.1 (0.370 g, 0.7258 mmol, 1.0 equiv), methanol (10 mL) and 10% palladium on carbon (0.10 g) was stirred under hydrogen for 1.5 h. The reaction mixture was filtered through a pad of Celite® and rinsed with methanol. The filtrate was concentrated under reduced pressure. The residue was purified by flash column chromatography on silica gel (CombiFlash®, 1.5% methanol in DCM) to afford (±)-cis-105.2 (0.260 g, 95%). MS(ES): m/z 376.25 [M+H]⁺.

Synthesis of compound (±)-cis-105.3. Compound (±)-cis-105.3 was prepared from compound (±)-cis-105.2 and T-1 following the procedure described in the synthesis of I-9. The product was purified by flash column chromatography on silica gel (1.5% methanol in DCM). MS(ES): m/z 593.28 [M+H]⁺.

Synthesis of compound (±)-cis-105. Compound (±)-cis-105 was prepared from compound (±)-cis-105.3 following the procedure described in the synthesis of I-11. The product was purified by flash column chromatography on silica gel (CombiFlash®, 3.2% methanol in DCM). MS(ES): m/z: 464.19 [M+H]⁺.

I-105-a and I-105-b. The racemate (±)-cis-105 was separated by chiral SFC (Method D) to afford first eluting fraction (I-105-a) and second eluting fraction (I-105-b).

I-105-a: MS(ES): m/z: 464.45 [M+H]⁺, ¹H NMR (DMSO-d₆, 400 MHz): δ 13.32 (s, 1H), 8.44 (s, 1H), 8.18 (s, 1H), 8.03 (s, 1H), 7.94 (s, 1H), 7.91 (s, 1H), 7.54 (s, 1H), 4.57-4.46 (m, 1H), 4.47-4.29 (m, 1H), 3.59 (s, 3H), 3.20-3.12 (m, 2H), 289 (s, 3H), 2.18-2.09 (m, 1H), 1.89-1.80 (m, 1H), 1.69-1.77 (m, 1H), 1.63-1.56 (m, 1H), 1.00-0.98 (d, J=6.4 Hz, 3H).

I-105-b: MS(ES): m/z: 464.47 [M+H]⁺, ¹H NMR (DMSO-d₆, 400 MHz): δ 13.34 (s, 1H), 8.44 (s, 1H), 8.18 (s, 1H), 8.03 (s, 1H), 7.94 (s, 1H), 7.91 (s, 1H), 7.54 (s, 1H), 4.56-4.47 (m, 1H), 4.39-4.29 (m, 1H), 3.59 (s, 3H), 3.20-3.12 (m, 2H), 2.89 (s, 3H), 2.18-2.09 (m, 1H), 1.89-1.80 (m, 1H), 1.69-1.77 (m, 1H), 1.63-1.56 (m, 1H), 1.00-0.98 (d, J=6.4 Hz, 3H).

The following intermediates were prepared following the procedure of I-105:

| Cmpd. | | |
|---|---|---|
| No. | Structure and Name | Analytical Data |
| I-127-aª and I- 127-b^{b} | 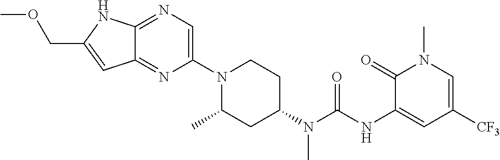 [SMILES: COCc1cc2nc(N3CC[C@H](N(C)C(=O)Nc4cc(C(F)(F)F)cn(C)c4=O)C[C@@H]3C)cnc2[nH]1] 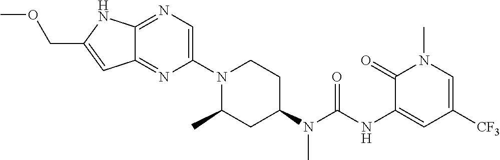 [SMILES: COCc1cc2nc(N3CC[C@@H](N(C)C(=O)Nc4cc(C(F)(F)F)cn(C)c4=O)C[C@H]3C)cnc2[nH]1] 1-((2S,4S)-1-(6-(methoxymethyl)-5H-pyrrolo[2,3- b]pyrazin-2-yl)-2-methylpiperidin-4-yl)-1-methyl- 3-(1-methyl-2-oxo-5-(trifluoromethyl)-1,2- dihydropyridin-3-yl)urea; 1-((2R,4R)-1-(6-(methoxymethyl)-5H-pyrrolo[2,3- b]pyrazin-2-yl)-2-methylpiperidin-4-yl)-1-methyl- 3-(1-methyl-2-oxo-5-(trifluoromethyl)-1,2- dihydropyridin-3-yl)urea | MS (ES): m/z 508.14 [M + H]⁺ ¹H NMR (DMSO-d₆, 400 MHz): δ 11.91 (s, 1H), 8.16 (s, 1H), 8.03 (s, 1H), 7.92 (s, 1H), 7.90 (s, 1H), 6.42 (s, 1H), 4.56 (s, 2H), 4.20 (s, 1H), 3.58 (s, 3H), 3.49-3.46 (m, 2H), 3.31 (s, 3H), 3.18- 3.16 (m, 1H), 2.91 (s, 3H), 1.86-1.84 (m, 1H), 1.73- 1.67 (m, 3H), 1.00-0.98 (d, J = 6.0 Hz, 3H) |
| I-132-a^{c} and I- 132-b^{d} | 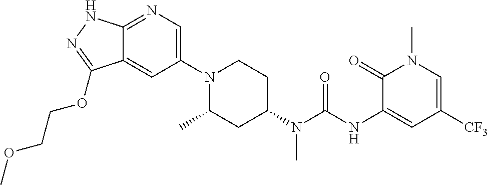 [SMILES: COCCOc1n[nH]c2ncc(N3CC[C@H](N(C)C(=O)Nc4cc(C(F)(F)F)cn(C)c4=O)C[C@@H]3C)cc12] 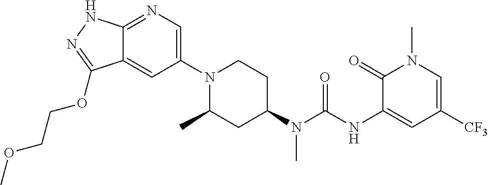 [SMILES: COCCOc1n[nH]c2ncc(N3CC[C@@H](N(C)C(=O)Nc4cc(C(F)(F)F)cn(C)c4=O)C[C@H]3C)cc12] 1-((2S,4S)-1-(3-(2-methoxyethoxy)-1H- pyrazolo[3,4-b]pyridin-5-yl)-2-methylpiperidin-4- yl)-1-methyl-3-(1-methyl-2-oxo-5- (trifluoromethyl)-1,2-dihydropyridin-3-yl)urea; 1-((2R,4R)-1-(3-(2-methoxyethoxy)-1H- pyrazolo[3,4-b]pyridin-5-yl)-2-methylpiperidin-4- yl)-1-methyl-3-(1-methyl-2-oxo-5- (trifluoromethyl)-1,2-dihydropyridin-3-yl)urea | MS (ES): m/z 538.4 [M + H]⁺ ¹HNMR (DMSO-d₆, 400 MHz): δ 12.49 (s, 1H), 8.29 (s, 1H), 8.19 (s, 1H), 8.05 (s, 1H), 7.92 (s, 2H), 4.44 (m, 2H), 4.28 (m, 1H), 3.75 (m, 2H), 3.61 (s, 3H), 3.35 (s, 3H), 3.18 (m, 2H), 2.93 (m, 4H), 1.88 (m, 1H), 1.73 (m, 1H), 1.66-1.60 (m, 2H), 0.82, (s, 3H) |
| I-133-a^{c} and I- 133-b^{d} | 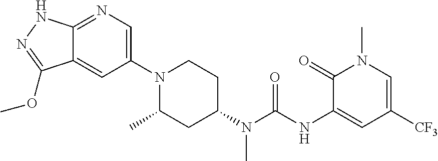 [SMILES: COc1n[nH]c2ncc(N3CC[C@H](N(C)C(=O)Nc4cc(C(F)(F)F)cn(C)c4=O)C[C@@H]3C)cc12] 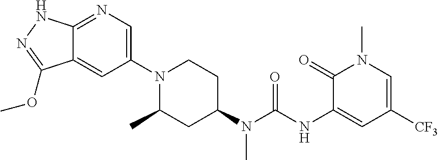 [SMILES: COc1n[nH]c2ncc(N3CC[C@@H](N(C)C(=O)Nc4cc(C(F)(F)F)cn(C)c4=O)C[C@H]3C)cc12] 1-((2S,4S)-1-(3-methoxy-1H-pyrazolo[3,4- b]pyridin-5-yl)-2-methylpiperidin-4-yl)-1-methyl- 3-(1-methyl-2-oxo-5-(trifluoromethyl)-1,2- dihydropyridin-3-yl)urea; 1-((2R,4R)-1-(3-methoxy-1H-pyrazolo[3,4- b]pyridin-5-yl)-2-methylpiperidin-4-yl)-1-methyl- 3-(1-methyl-2-oxo-5-(trifluoromethyl)-1,2- dihydropyridin-3-yl)urea | MS (ES): m/z 494.43 [M + H]⁺ ¹H NMR (DMSO-d₆, 400 MHz): δ 12.20 (s, 1H), 8.39 (s, 1H), 8.18 (s, 1H), 8.02 (s, 1H), 7.91 (s, 1H), 7.37-7.36 (d, J = 2.0 Hz, 1H), 4.55-4.42 (m, 1H), 4.23-4.33 (m, 1H), 3.97 (s, 3H), 3.59 (s, 3H), 3.17-3.12 (m, 1H), 2.89 (s, 3H), 2.18-2.06 (m, 1H), 1.87- 1.54 (m, 2H), 0.98-0.97 (d, J = 6.8 Hz, 3H) |

^{a}Separated by chiral SFC (Method D); first eluting isomer;
^{b}Separated by chiral SFC (Method D); second eluting isomer;
^{c}Separated by chiral HPLC (Method C); first eluting isomer;
^{d}Separated by chiral HPLC (Method C); second eluting isomer.

### Example 106: 1-((3S,4R)-3-methoxy-1-(3-methoxy-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-((3R,4S)-3-methoxy-1-(3-methoxy-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

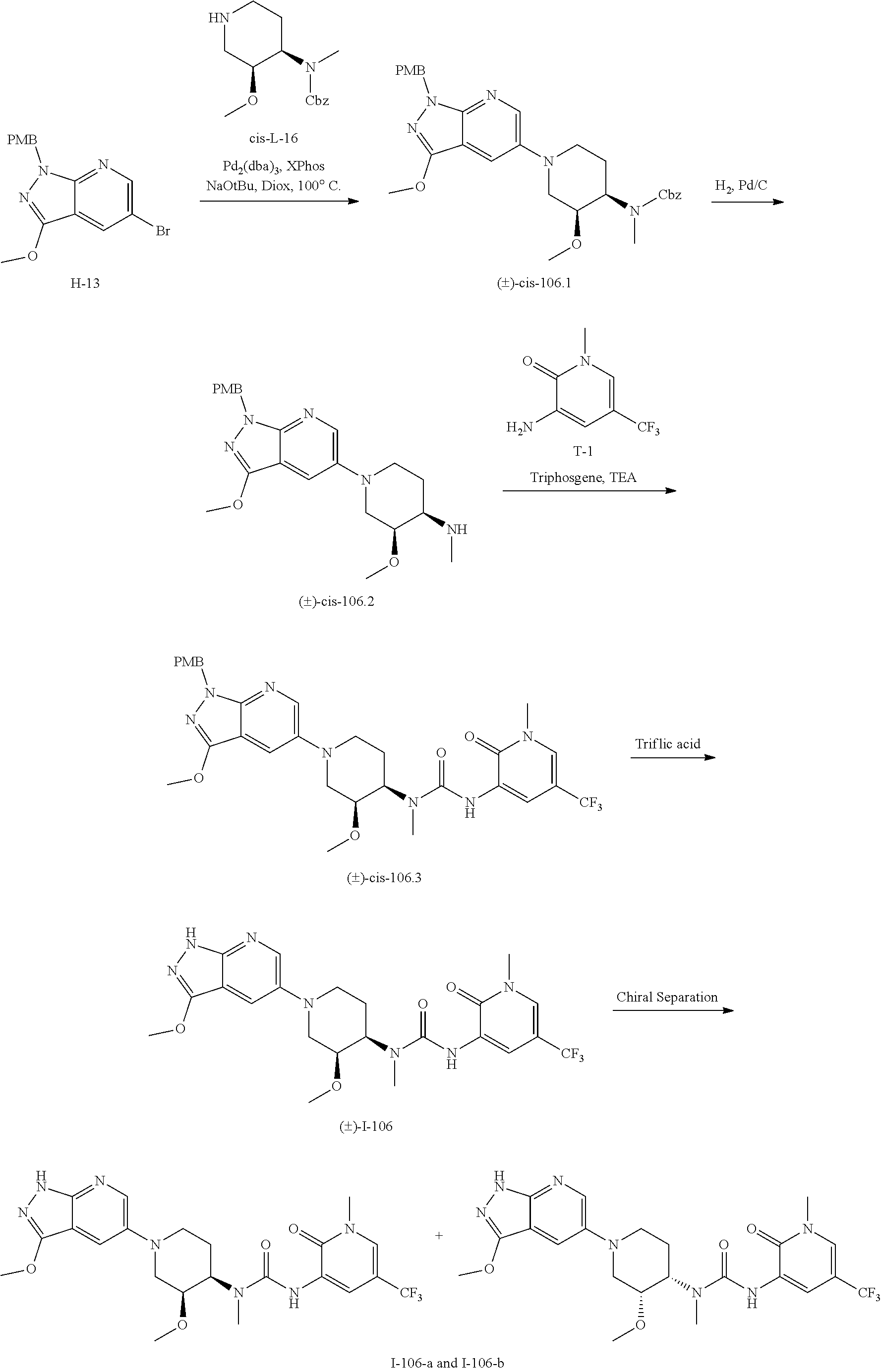

Synthesis of compound (±)-cis-106.1. Compound (±)-cis-106.1 was prepared from compound H-13 and cis-L-16 following the procedure described in the synthesis of 11.1. The product was purified by flash column chromatography on silica gel (CombiFlash®, 40% ethyl acetate in hexane). MS(ES): m/z 546.64 [M+H]⁺.

Synthesis of compound (±)-cis-106.2. A mixture of (±)-cis-106.1 (0.310 g, 568.14 mmol, 1.0 equiv), methanol (10 mL) and 10% palladium on carbon (0.150 g) was stirred under hydrogen (1 atm) for 5 h. The reaction mixture was filtered through a pad of Celite® and rinsed with methanol. The filtrate was concentrated under reduced pressure to afford (±)-cis-106.2 (0.240 g, 100%). MS(ES): m/z 412.51 [M+H]⁺.

Synthesis of compound (±)-cis-106.3. Compound (±)-cis-106.3 was prepared from compound (±)-cis-106.2 and T-1 following the procedure described in the synthesis of I-9. The product was purified by flash column chromatography on silica gel (2.5% methanol in DCM). MS(ES): m/z 630.26 [M+H]⁺.

Synthesis of compound (±)-cis-106. To a stirred solution of (±)-cis-106.3 (0.152 g, 0.241 mmol, 1 equiv) in DCM (5 mL) at 0° C. was added triflic acid (1.0 mL) and allowed to stir at room temperature for 2 h. The reaction mixture was poured into a saturated sodium bicarbonate solution extracted with DCM. The combined organic layers were washed with brine, dried over anhydrous sodium sulfate, filtered, and concentrated under reduced pressure. The residue was purified by flash column chromatography on silica gel (CombiFlash®, 5.0% methanol in DCM) to afford (±)-cis-106 (0.095 g, 77%). MS(ES): m/z 510.49 [M+H]⁺.

I-106-a and I-106-b. The racemate (±)-cis-106 was separated by chiral HPLC (Method C) to isolate fraction-1 (I-106-a) and second eluting fraction (I-106-b).

I-106-a: MS(ES): m/z: 510.49 [M+H]⁺, ¹H NMR (DMSO-d₆, 400 MHz): δ 12.25 (s, 1H), 8.41-8.40 (d, J=2.8 Hz, 1H), 8.18-8.17 (d, J=2.0 Hz, 1H), 8.13 (s, 1H), 8.03 (s, 1H), 7.45-7.44 (d, J=2.4 Hz, 1H), 4.19-4.15 (m, 1H), 3.97 (s, 3H), 3.89-3.85 (m, 1H), 3.68-3.58 (m, 2H), 3.53 (s, 3H), 3.31 (s, 3H), 3.02 (s, 3H), 2.86-2.80 (m, 1H), 2.73-2.70 (m, 1H), 2.38-2.32 (m, 1H), 1.57-1.55 (m, 1H).

I-106-b: MS(ES): m/z: 510.49 [M+H]⁺, ¹H NMR (DMSO-d₆, 400 MHz): δ 12.25 (s, 1H), 8.41-8.40 (d, J=2.8 Hz, 1H), 8.18-8.17 (d, J=2.0 Hz, 1H), 8.13 (s, 1H), 8.03 (s, 1H), 7.45-7.44 (d, J=2.4 Hz, 1H), 4.19-4.15 (m, 1H), 3.97 (s, 3H), 3.89-3.85 (m, 1H), 3.68-3.58 (m, 2H), 3.53 (s, 3H), 3.31 (s, 3H), 3.02 (s, 3H), 2.86-2.80 (m, 1H), 2.73-2.70 (m, 1H), 2.38-2.32 (m, 1H), 1.57-1.55 (m, 1H).

The following compounds were prepared following the procedure of 1-106:

| Cmpd. | | |
|---|---|---|
| No. | Structure and Name | Analytical Data |
| I-154-aª and I- 154-b^{b} | 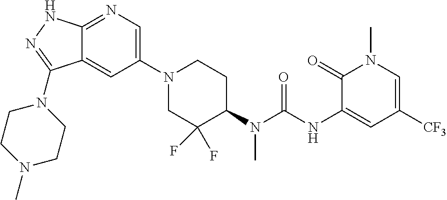 [SMILES: CN1CCN(c2n[nH]c3ncc(N4CC[C@@H](N(C)C(=O)Nc5cc(C(F)(F)F)cn(C)c5=O)C(F)(F)C4)cc23)CC1] 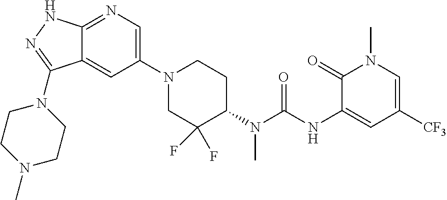 [SMILES: CN1CCN(c2n[nH]c3ncc(N4CC[C@H](N(C)C(=O)Nc5cc(C(F)(F)F)cn(C)c5=O)C(F)(F)C4)cc23)CC1] (R)- or (S)-1-(3,3-difluoro-1-(3-(4-methylpiperazin- 1-yl)-1H-pyrazolo[3,4-b]pyridin-5-yl)piperidin-4-yl)- 1-methyl-3-(1-methyl-2-oxo-5-(trifluoromethyl)-1,2- dihydropyridin-3-yl)urea | MS (ES): m/z 584.45 [M + H]⁺ ¹H NMR (DMSO-d₆, 400 MHz): δ 12.35 (s, 1H), 8.37- 8.36 (d, J = 2.4 Hz, 1H), 8.15-8.15 (d, J = 2.0 Hz, 1H), 8.07 (s, 2H), 7.73-7.72 (d, J = 4.0 Hz, 1H), 4.79(bs, 1H), 3.87 (bs, 1H), 3.73-3.71 (bs, 1H), 3.60 (s, 3H), 3.33- 3.18 (m, 3H), 3.05-3.01 (m, 5H), 2.33-2.29 (m, 2H), 2.25 (s, 3H), 1.87-1.84 (m, 2H), 1.33 (s, 1H), 1.23 (s, 2H) |
| I-162-a^{c} and I- 162-b^{d} | 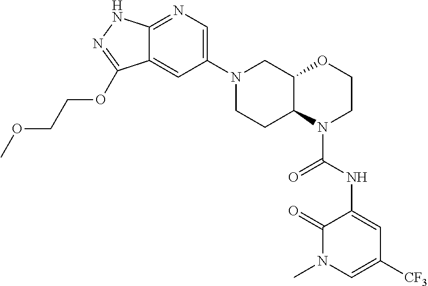 [SMILES: COCCOc1n[nH]c2ncc(N3CC[C@H]4[C@H](C3)OCCN4C(=O)Nc3cc(C(F)(F)F)cn(C)c3=O)cc12] 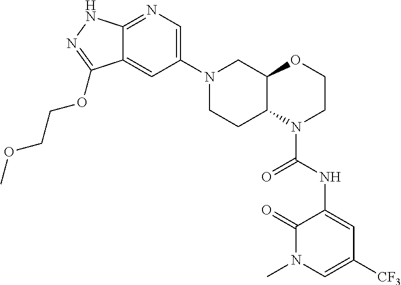 [SMILES: COCCOc1n[nH]c2ncc(N3CC[C@@H]4[C@@H](C3)OCCN4C(=O)Nc3cc(C(F)(F)F)cn(C)c3=O)cc12] (4aS,8aS)-6-(3-(2-methoxyethoxy)-1H-pyrazolo[3,4- b]pyridin-5-yl)-N-(1-methyl-2-oxo-5- (trifluoromethyl)-1,2-dihydropyridin-3-yl)octahydro- 1H-pyrido[3,4-b][1,4]oxazine-1-carboxamide; (4aR,8aR)-6-(3-(2-methoxyethoxy)-1H-pyrazolo[3,4- b]pyridin-5-yl)-N-(1-methyl-2-oxo-5- (trifluoromethyl)-1,2-dihydropyridin-3-yl)octahydro- 1H-pyrido[3,4-b][1,4]oxazine-1-carboxamide | MS (ES): m/z 552.45 [M + H]⁺ ¹H NMR (DMSO-d₆, 400 MHz): δ 12.30 (s, 1H), 8.44- 8.43 (d, J = 2.8 Hz, 1H), 8.30 (S, 1H), 8.10-8.09 (d, J = 2.8 Hz, 1H,), 8.06 (s, 1H), 7.46- 7.45 (d, J = 2.4 Hz, 1H), 4.43- 4.41 (t, J = 2.8 Hz, 2H), 3.91-3.95 (m, 1H), 3.89-3.83 (m, 1H), 3.73-3.71 (m, 4H), 3.61-3.60 (m, 1H), 3.58 (s, 3H), 3.40-3.34 (m, 1H), 3.32 (s, 3H), 3.25-3.23 (m, 1H), 2.81-2.75(m, 1H ), 2.70- 2.64(m, 2H), 1.76-1.72 (m, 1H), 1.36-1.31(m, 1H) |
| I-169-aª and I- 169-b^{b} | 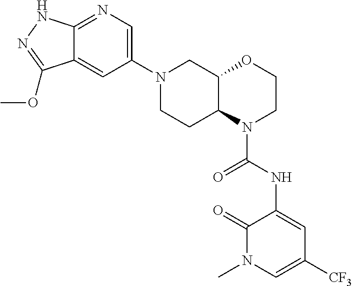 [SMILES: COc1n[nH]c2ncc(N3CC[C@H]4[C@H](C3)OCCN4C(=O)Nc3cc(C(F)(F)F)cn(C)c3=O)cc12] 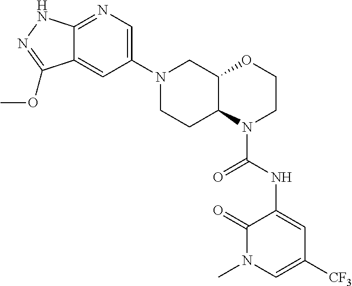 [SMILES: COc1n[nH]c2ncc(N3CC[C@H]4[C@H](C3)OCCN4C(=O)Nc3cc(C(F)(F)F)cn(C)c3=O)cc12] (4aS,8aS)-6-(3-methoxy-1H-pyrazolo[3,4-b]pyridin- 5-yl)-N-(1-methyl-2-oxo-5-(trifluoromethyl)-1,2- dihydropyridin-3-yl)octahydro-1H-pyrido[3,4- b][1,4]oxazine-1-carboxamide; (4aR,8aR)-6-(3-methoxy-1H-pyrazolo[3,4-b]pyridin- 5-yl)-N-(1-methyl-2-oxo-5-(trifluoromethyl)-1,2- dihydropyridin-3-yl)octahydro-1H-pyrido[3,4- b][1,4]oxazine-1-carboxamide | MS (ES): m/z 508.26 [M + H]⁺ ¹H NMR (DMSO-d₆, 400 MHz): δ 12.27 (s, 1H), 8.43- 8.42 (d, J = 2.8 Hz, 1H), 8.27 (s, 1H), 8.09 (s, 1H), 8.05 (s, 1H), 7.47 (s, 1H), 3.97 (s, 3H), 3.94-3.91 (m, 1H), 3.82-3.76 (m, 2H), 3.72-3.68 (m, 2H), 3.57 (s, 3H), 3.41- 3.37 (m, 1H), 3.32-3.22 (m, 1H), 2.79-2.73 (m, 1H), 2.68-2.63 (m, 2H), 1.78-1.72 (m, 2H) |

^{a}Separated by chiral SFC (Method D); first eluting isomer;
^{b}Separated by chiral SFC (Method D); second eluting isomer;
^{c}Separated by chiral HPLC (Method K); first eluting isomer;
^{d}Separated by chiral HPLC (Method K); second eluting isomer.

### Example 107: 1-((3S,4R)-3-fluoro-1-(3-((tetrahydro-2H-pyran-4-yl)oxy)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-((3R,4S)-3-fluoro-1-(3-((tetrahydro-2H-pyran-4-yl)oxy)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

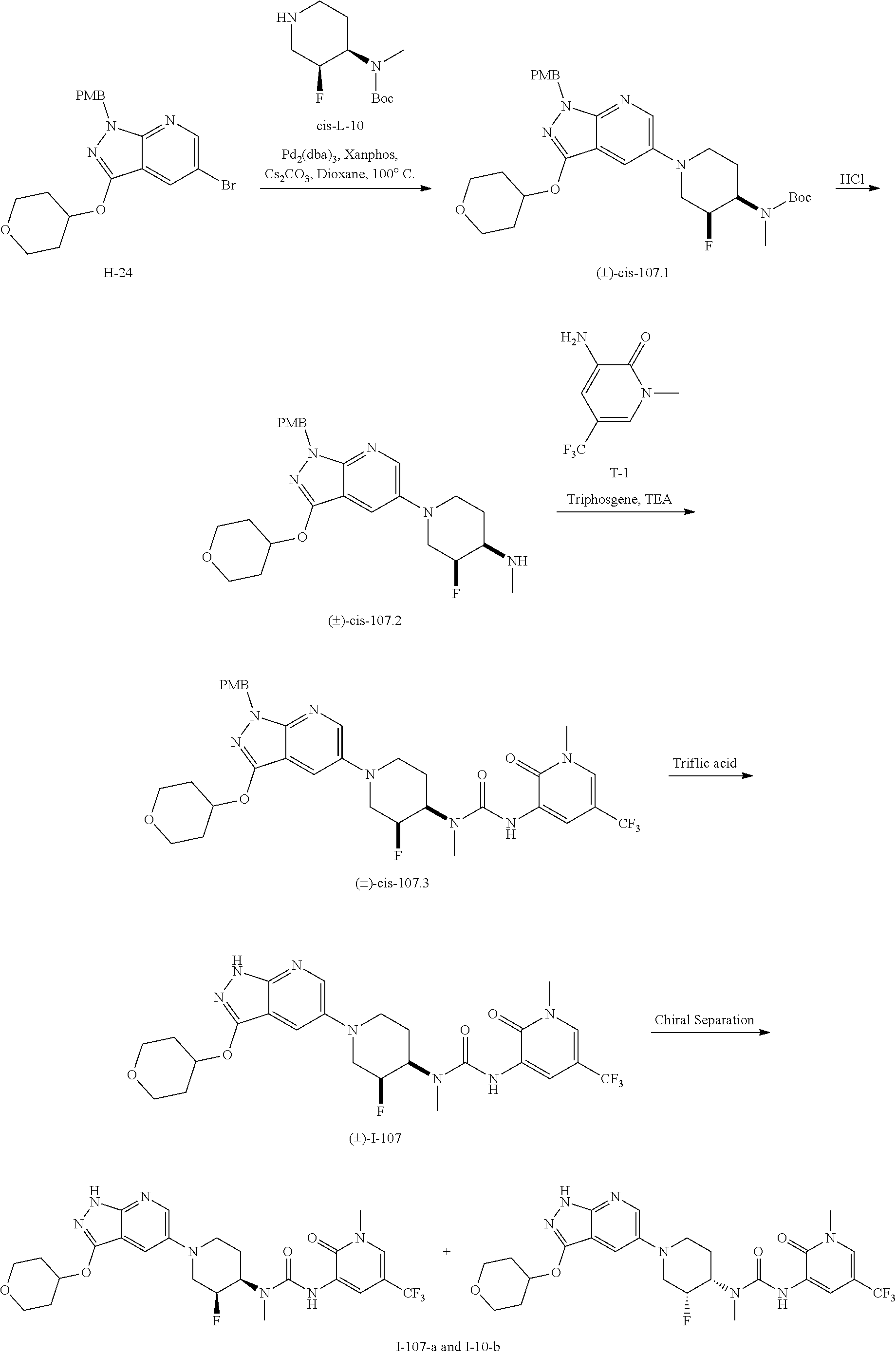

Synthesis of compound (±)-cis-107.1. Compound (±)-cis-107.1 was prepared from H-24 and cis-L-10 following the procedure described in the synthesis of (±)-96.1. The product was purified by flash column chromatography on silica gel (CombiFlash®, 35% ethyl acetate in hexane). MS(ES): m/z 570.30 [M+H]⁺.

Synthesis of compound (±)-cis-107.2. To a solution of (±)-cis-107.1 (0.4 g, 2.09 mmol, 1.0 equiv) in DCM (0.7 mL) was added 4 M hydrochloric acid in 1,4-dioxane (0.9 mL) dropwise at 0° C. The reaction mixture was stirred at room temperature for 1 h. It was concentrated under reduced pressure to afford (±)-cis-107.2 (0.35 g, 100%). MS(ES): m/z 470.25 [M+H]⁺.

Synthesis of compound (±)-cis-107.3. Compound (±)-cis-107.3 was prepared from compound (±)-cis-107.2 and T-1 following the procedure described in the synthesis of I-9. The product was purified by flash column chromatography on silica gel (CombiFlash®, 3.5% methanol in DCM). MS(ES): m/z 688.28 [M+H]⁺.

Synthesis of compound (±)-cis-107. To a solution of (±)-cis-107.3 (0.25 g, 0.36 mmol, 1.0 equiv) in DCM (2.0 mL) was added triflic acid (0.5 mL) at 0° C. The reaction mixture was stirred for 30 min. It was poured over ice water, basified with saturated sodium bicarbonate solution, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3-3.5% methanol in DCM) to afford (z)-cis-107 (0.11 g, 53%). MS(ES): m/z 568.23 [M+H]⁺.

I-107-a and I-107-b. The racemate was separated by chiral SFC (Method F) to afford first eluting fraction (1-107-a) and second eluting fraction (1-107-b).

I-107-a: (MS(ES): m/z: 568.37 [M+H], 3H-NMR (400 MHz, DMSO-d₆): δ 12.30 (s, 1H), 8.40 (s, 1H), 8.18 (s, 1H), 8.06 (s, 1H), 7.98 (s, 1H), 7.45 (s, 1H), 4.97 (s, 1H), 4.97-4.85 (d, J_{HF}=48.4 Hz, 1H), 4.47-4.32 (m, 1H), 3.91-3.85 (m, 2H), 3.82-3.73 (s, 2H), 3.59 (s, 3H), 3.51-3.47 (i, 2H), 3.11-3.90 (N, 2H), 2.98 (s, 3H), 2.38-2.32 (i, 1H), 2.11-2.08 (s, 2H), 1.71-1.68 (m, 2H).

I-107-b: MS(ES): m/z: 569.62 [M+H]⁺, ¹H-NMR (400 MHz, DMSO-d₆): δ 12.29 (s, 1H), 8.40 (s, 1H), 8.17 (s, 1H), 8.05 (s, 1H), 7.98 (s, 1H), 7.45 (s, 1H), 4.96 (m, 1H), 4.96-4.84 (d, J_{HF}=48.4 Hz, 1H), 4.45-4.33 (m, 2H), 3.91-3.85 (m, 2H), 3.82-3.72 (m, 2H), 3.59 (s, 3H), 3.51-3.47 (ti, 2H), 3.11-3.90 (m, 2H), 2.98 (s, 3H), 2.38-2.32 (m, 1H), 2.11-2.08 (i, 2H), 1.70-1.68 (id, 2H).

The following compounds were prepared following the procedure of 1-107:

| Cmpd. | | |
|---|---|---|
| No. | Structure and Name | Analytical Data |
| I-114-aª and I- 114-b^{b} | 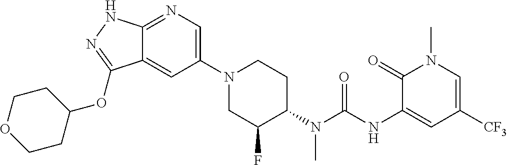 [SMILES: CN(C(=O)Nc1cc(C(F)(F)F)cn(C)c1=O)[C@H]1CCN(c2cnc3[nH]nc(OC4CCOCC4)c3c2)C[C@@H]1F] 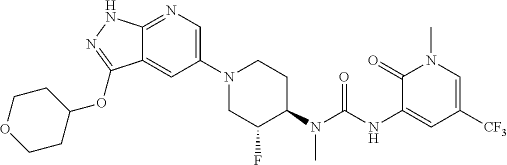 [SMILES: CN(C(=O)Nc1cc(C(F)(F)F)cn(C)c1=O)[C@@H]1CCN(c2cnc3[nH]nc(OC4CCOCC4)c3c2)C[C@H]1F] 1-((3S,4S)-3-fluoro-1-(3-((tetrahydro-2H-pyran- 4-yl)oxy)-1H-pyrazolo[3,4-b]pyridin-5- yl)piperidin-4-yl)-1-methyl-3-(1-methyl-2-oxo-5- (trifluoromethyl)-1,2-dihydropyridin-3-yl)urea; 1-((3R,4R)-3-fluoro-1-(3-((tetrahydro-2H-pyran- 4-yl)oxy)-1H-pyrazolo[3,4-b]pyridin-5- yl)piperidin-4-yl)-1-methyl-3-(1-methyl-2-oxo-5- (trifluoromethyl)-1,2-dihydropyridin-3-yl)urea | MS (ES): m/z 568.15 [M + H]⁺ ¹H NMR (DMSO-d₆, 400 MHz): δ 12.34 (s, 1H), 8.45 (s, 1H), 8.15 (s, 1H), 8.05 (s, 1H), 7.99 (s, 1H), 7.54 (s, 1H), 4.99- 4.94 (m, 1H), 4.84-4.80 (m, 1H), 4.31 (m, 1H), 3.99-3.98 (m, 1H), 3.93-3.88 (m, 2H), 3.59 (s, 3H), 3.53-3.52 (m, 1H), 3.49-3.46 (m, 2H), 2.99 (s, 3H), 2.84-2.78 (m, 2H), 2.11-2.08 (m, 2H), 1.99-1.96 (m, 1H), 1.80 (m, 1H), 1.76-1.66 (m, 2H) |

^{a}Separated by chiral SFC (Method H); first eluting isomer;
bSeparated by chiral SFC (Method H); second eluting isomer.

### Example 116: 1-(3-(1H-pyrazolo[3,4-b]pyridin-5-yl)bicyclo[1.1.1]pentan-1-yl)-1-methyl-3-(1-methyl-2-oxo-5-(trifluoromethyl)-1,2-dihydropyridin-3-yl)urea

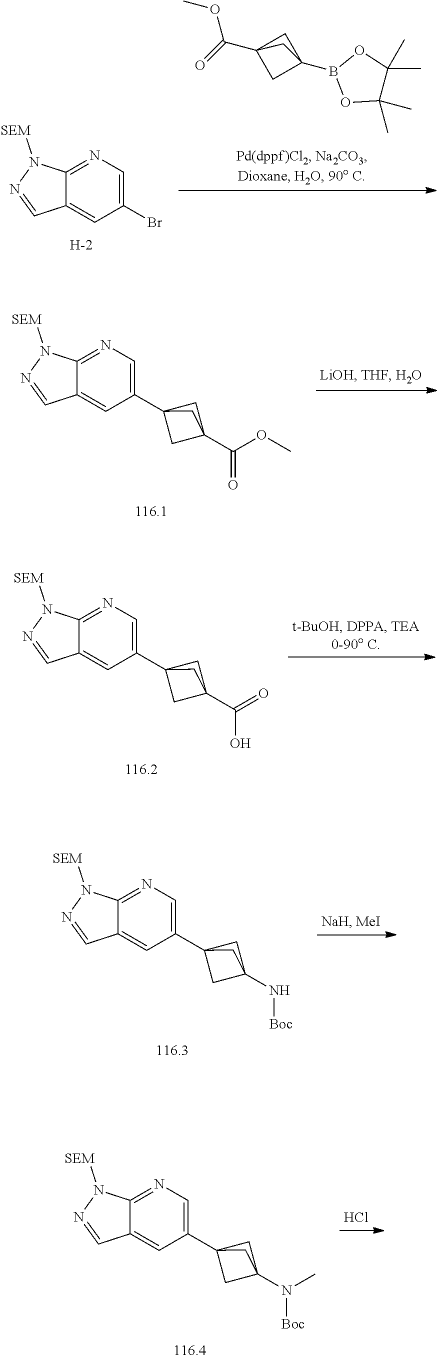

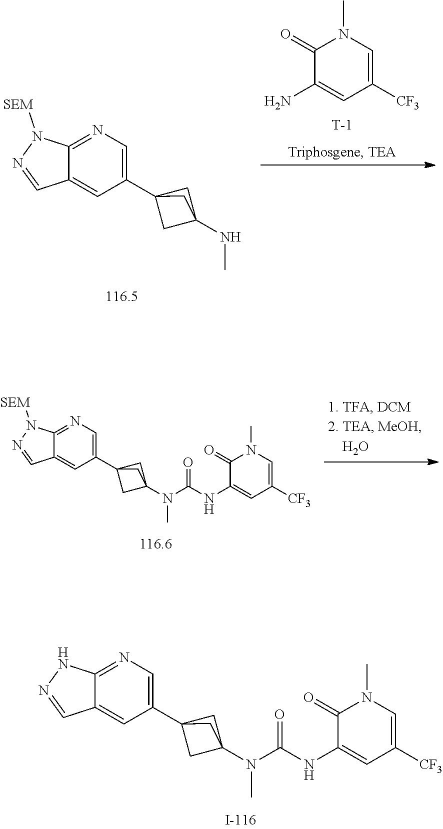

Synthesis of compound 116.1. A mixture of H-2 (0.5 g, 1.51 mmol, 1.0 equiv), methyl 3-(4,4,5,5-tetramethyl-1,3,2-dioxaborolan-2-yl)bicyclo[1.1.1]pentane-1-carboxylate (0.55 g, 2.27 mmol, 1.5 equiv) and sodium carbonate (0.472 g, 4.45 mmol, 3.0 equiv) in 1-4, dioxane (5.0 mL) was degassed by bubbling through a stream of argon for 10 min. 1,1′-Bis(diphenylphosphino)ferrocene)palladium(II) dichloride (0.110 g, 0.151 mmol, 0.1 equiv) was added and degassed for 5 min. The reaction mixture was stirred at 90° C. for 12 h. It was cooled to room temperature, transferred into water, extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford 116.1 (0.320 g, 56.%). MS(ES): m/z 374.18 [M+H]⁺.

Synthesis of compound 116.2. To a solution of 116.1 (0.30 g, 0.80 mmol, 1.0 equiv), in THF: water (5.0 mL, 2:1) was added lithium hydroxide (0.92 g, 4.01 mmol, 5.0 equiv). The reaction mixture was stirred at room temperature for 48 h. It was concentrated under reduced pressure. To the residue was added water and acidified with 1N hydrochloric acid to adjust pH-6-6.5 at 10° C. The mixture was extracted with 10% methanol in DCM. The combined organic layes were dried over anhydrous sodium sulfate, filtered, and concentrated under reduced pressure to afford 116.2 (0.195 g, 67%). MS(ES): m/z 360.17 [M+H]⁺.

Synthesis of compound 116.3. A mixture of 116.2 (0.195 g, 0.54 mmol, 1.0 equiv, triethylamine (0.1 mL, 1.92 mmol, 1.7 equiv) and diphenyl phosphoryl azide (0.15 mL, 0.70 mmol, 1.3 equiv) in tert-butanol (3.0 mL) was stirred under nitrogen at 80° C. for 16 h. It cooled to room temperature, transferred into water, and extracted with ethyl acetate. The combined organic layers were washed with brine, dried over anhydrous sodium sulfate, filtered, and concentrated under reduced pressure. The residue was purified by by flash column chromatography on silica gel (2.5% methanol in DCM) to afford 116.3 (0.09 g, 39%). MS(ES): m/z 431.24 [M+H]⁺.

Synthesis of compound 116.4. To a solution of 116.3 (0.090 g, 0.20 mmol, 1.0 equiv) in DMF (3.0 mL) was added sodium hydride (0.009 g, 0.62 mmol, 3.0 equiv) at 0° C. and stirred for 30 min. Methyl iodide (0.042 g, 0.30 mmol, 1.5 equiv) was added dropwise and stirred at room temperature for 1 h. It was added to cold water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.5% methanol in DCM) to afford 116.4 (0.057 g, 64%). MS (ES): m/z 445.26 [M+H]⁺.

Synthesis of compound 116.5. To a solution of 116.4 (0.057 g, 0.12 mmol, 1.0 equiv) in DCM (0.5 mL) was added 4 M hydrochloric acid in 1,4-dioxane (2.0 mL) dropwise at 0° C. The reaction mixture was stirred at room temperature for 20 min. It was concentrated to afford 116.5 (0.05 g, 100%). MS(ES): m/z 345.20 [M+H]⁺.

Synthesis of compound 116.6. Compound 116.6 was prepared from compound 116.5 and T-1 following the procedure described in the synthesis of I-9. The product was purified by flash column chromatography on silica gel (CombiFlash®, 35% ethyl acetate in hexane. MS(ES): m/z 563.23 [M+H]⁺.

Synthesis of I-116. Compound I-116 was prepared from compound 116.6 following the procedure described in the synthesis of I-11. The product was purified by flash column chromatography on silica gel (CombiFlash®, 3.8% methanol in DCM). MS(ES): m/z 433.15 [M+H]⁺, ¹H NMR (DMSO-d₆, 400 MHz): δ 13.38 (s, 1H), 8.55 (s, 1H), 8.46 (s, 1H), 7.97 (s, 1H), 7.76 (s, 1H), 7.64 (s, 1H), 7.10 (s, 1H), 2.88 (s, 3H), 2.82-2.76 (8, 2H), 2.34 (s, 3H), 1.94-1.91 (m, 2H), 1.71-1.68 (m, 2H).

The following compounds were prepared following the procedure of I-116:

| Cmpd. | | |
|---|---|---|
| No. | Structure and Name | Analytical Data |
| I-121 | 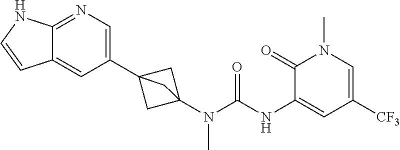 [SMILES: CN(C(=O)Nc1cc(C(F)(F)F)cn(C)c1=O)C12CC(c3cnc4[nH]ccc4c3)(C1)C2] 1-(3-(1H-pyrrolo[2,3-b]pyridin-5- yl)bicyclo[1.1.1]pentan-1-yl)-1-methyl- 3-(1-methyl-2-oxo-5-(trifluoromethyl)- 1,2-dihydropyridin-3-yl)urea | MS (ES): m/z 433.15 [M + H]⁺ ¹H NMR (DMSO-d₆, 400 MHz): δ 11.90 (s, 1H), 8.29 (s, 1H), 8.16-8.15 (d, J = 2.0 Hz, 1H), 8.01 (s, 1H), 7.83 (s, 1H), 7.80- 7.79 (t, J = 2.8 Hz, 1H), 6.60-6.59 (d, J = 1.6 Hz, 1H), 3.57 (s, 3H), 2.72 (s, 3H), 2.44-2.40 (m, 2H), 1.89-1.77 (m, 2H), 1.52-1.45 (m, 2H) |
| I-156 | 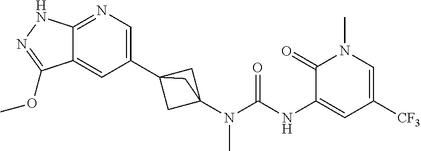 [SMILES: COc1n[nH]c2ncc(C34CC(N(C)C(=O)Nc5cc(C(F)(F)F)cn(C)c5=O)(C3)C4)cc12] 1-(3-(3-methoxy-1H-pyrazolo[3,4- b]pyridin-5-yl)bicyclo[1.1.1]pentan-1- yl)-1-methyl-3-(1-methyl-2-oxo-5- (trifluoromethyl)-1,2-dihydropyridin-3- yl)urea | MS (ES): m/z 463.42 [M + H]⁺ ¹H NMR (DMSO-d₆, 400 MHz): δ 12.27 (s, 1H), 8.41-8.40 (d, J = 2.4 Hz, 1H), 8.18-8.17 (d, J = 2.4 Hz, 1H), 8.05 (s, 1H), 7.98 (s, 1H), 7.49-7.48 (d, J = 2.4 Hz, 1H), 4.97-4.85 (m, 1H), 3.98 (s, 3H), 2.87 (s, 3H), 2.77-2.74 (m, 2H), 2.34 (s, 3H) 1.93- 1.90 (m, 2H), 1.68.1.64 (m, 2H) |
| I-157 | 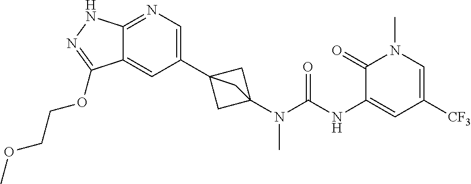 [SMILES: COCCOc1n[nH]c2ncc(C34CC(N(C)C(=O)Nc5cc(C(F)(F)F)cn(C)c5=O)(C3)C4)cc12] 1-(3-(3-(2-methoxyethoxy)-1H- pyrazolo[3,4-b]pyridin-5- yl)bicyclo[1.1.1]pentan-1-yl)-1-methyl- 3-(1-methyl-2-oxo-5-(trifluoromethyl)- 1,2-dihydropyridin-3-yl)urea | MS (ES): m/z 507.42 [M + H]⁺ ¹H NMR (DMSO-d₆, 400 MHz): δ 12.28 (s, 1H), 8.55 (s, 1H), 7.76 (s, 1H), 7.61 (s, 1H), 7.44-7.43 (d, J = 2.4 Hz, 1H), 7.10 (s, 1H), 4.43-4.41 (m, 2H), 3.73-3.71 (m, 2H ) 3.67-3.64 (m, 2H), 3.34 (s, 3H), 2.87 (s, 3H), 2.80-2.74 (m, 2H), 2.34 (s, 3H), 1.93- 1.89 (m, 2H), 1.69-1.66 (m, 2H) |
| I-158 | 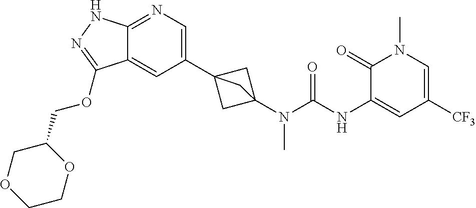 [SMILES: CN(C(=O)Nc1cc(C(F)(F)F)cn(C)c1=O)C12CC(c3cnc4[nH]nc(OC[C@H]5COCCO5)c4c3)(C1)C2] (R)-1-(3-(3-((1,4-dioxan-2-yl)methoxy)- 1H-pyrazolo[3,4-b]pyridin-5- yl)bicyclo[1.1.1]pentan-1-yl)-1-methyl- 3-(1-methyl-2-oxo-5-(trifluoromethyl)- 1,2-dihydropyridin-3-yl)urea | MS (ES): m/z 549.47 [M + H]⁺ ¹H NMR (DMSO-d₆, 400 MHz): δ 12.31 (s, 1H), 8.55 (s, 1H), 7.76 (s, 1H), 7.61 (s, 1H), 7.47-7.46 (d, J = 2.4 Hz, 1H), 7.10 (s,1H), 4.28-4.26 (m, 2H), 4.22-4.17 (m, 1H), 3.95-3.28 (m, 2H) 3.80-3.76 (m, 2H), 3.67-6.61 (m, 2H), 2.88 (s, 3H), 2.80-2.75 (m, 2H), 2.34 (s, 3H), 1.95-1.97 (m, 2H), 1.69.1.66 (m, 2H) |
| I-166 | 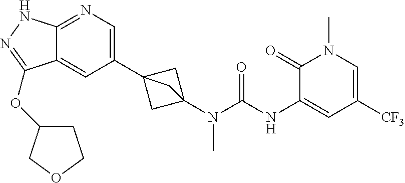 [SMILES: CN(C(=O)Nc1cc(C(F)(F)F)cn(C)c1=O)C12CC(c3cnc4[nH]nc(OC5CCOC5)c4c3)(C1)C2] 1-methyl-3-(1-methyl-2-oxo-5- (trifluoromethyl)-1,2-dihydropyridin-3- yl)-1-(3-(3-((tetrahydrofuran-3-yl)oxy)- 1H-pyrazolo[3,4-b]pyridin-5- yl)bicyclo[1.1.1]pentan-1-yl)urea | MS (ES): m/z 519.43 [M + H]⁺ ¹H NMR (DMSO-d₆, 400 MHz): δ 12.27 (s, 1H), 8.41-8.40 (d, J = 2.4 Hz, 1H), 8.18-8.17 (d, J = 2.4 Hz, 1H), 8.05 (s, 1H), 7.98 (s, 1H), 7.49-7.48 (d, J = 2.4 Hz, 1H), 5.40-5.36 (m, 1H), 3.93-3.87 (m, 2H), 3.80-3.76 (m, 1H), 3.67-3.64 (m, 1H), 2.87 (s, 3H), 2.79-2.74 (m, 2H), 2.34 (s, 3H), 2.27-2.24 (m, 1H), 2.17-2.13 (m, 1H), 1.93-1.90 (m, 2H), 1.68-1.66 (m, 2H) |

### Example 119: 1-(1-(2-((1r,3r)-3-methoxycyclobutyl)-3-oxo-2,3-dihydro-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

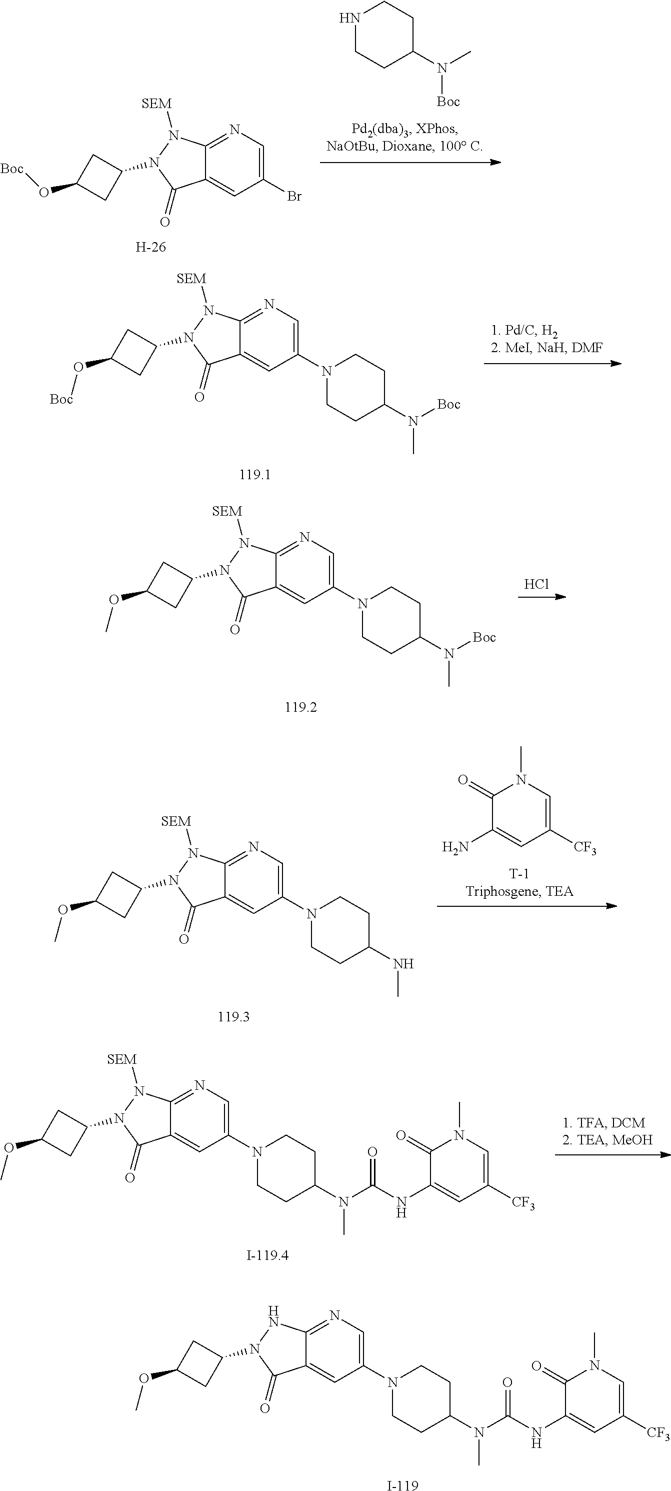

Synthesis of compound 119.1. Compound 119.1 was prepared from compound H-26 and tert-butyl methyl(piperidin-4-yl)carbamate following the procedure described in the synthesis of 11.1. The product was purified by flash column chromatography on silica gel (CombiFlash®, 30% ethyl acetate in hexane). MS(ES): m/z: 638.90 [M+H]⁺.

Synthesis of compound 119.2. A mixture of compound 119.1 (0.4 g, 0.627 mmol, 1.0 equiv), methanol (4 mL) and palladium on carbon (10 wt %, 0.2 g) was stirred under hydrogen for 30 min. The reaction mixture was filtered through a pad of Celite® and rinsed with methanol. The filtrate was concentrated under reduced pressure. The residue was used in the next step without purification.

Synthesis of compound 119.3. To a solution of 119.2 in DMF (6 mL) was added sodium hydride (60% in mineral oil, 0.083 g, 1.254 mmol, 2.0 equiv) in portions at 0° C. and stirred for 5 min. To the mixture was added methyl iodide (0.078 mL, 1.254 mmol, 2.0 equiv) and stirred for 30 min. The reaction mixture was quenched with water (150 mL) and extracted with ethyl acetate (200 mL). The combined organic layers were washed with brine (80 mL) and dried over anhydrous sodium sulfate, filtered, and concentrated. The residue was purified by flash column chromatography on silica gel (CombiFlash®, 32% ethyl acetate in hexane) to afford 119.3 (0.240 g, 68%). MS(ES): m/z: 562.80 [M+H]⁺.

Synthesis of compound 119.4. To a solution of 119.3 (0.240 g, 0.427 mmol, 1.0 equiv) in 1,4-dioxane (2.8 mL) was added 4 M hydrochloric acid in 1,4-dioxane (2.8 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 85% ethyl acetate in hexane) to afford 119.4 (0.20 g, 100%). MS(ES): m/z 462.68 [M+H]⁺.

Synthesis of compound 119.5. Compound 119.5 was prepared from compound 119.4 and T-1 following the procedure described in the synthesis of I-9. The product was purified by flash column chromatography on silica gel (3.7% methanol in DCM). MS(ES): m/z 680.82 [M+H]⁺.

Synthesis of I-119. Compound I-119 was prepared from compound 119.5 following the procedure described in the synthesis of I-11. The product was purified by flash column chromatography on silica gel (CombiFlash®, 3.2% methanol in DCM). MS(ES): m/z: 550.56 [M+H]⁺, ¹H NMR (DMSO-d₆, 400 MHz): δ 8.53 (s, 1H), 8.18 (s, 1H), 8.04 (s, 1H), 7.92 (s, 1H), 7.56 (s, 1H), 5.28-5.27 (d, J=6.4 Hz, 1H), 4.24-4.21 (m, 2H), 3.93-4.21 (m, 1H), 3.78-3.76 (m, 2H), 3.60 (s, 3H), 2.89 (s, 3H), 2.84-2.78 (m, 3H), 2.68-2.56 (m, 4H), 2.41-2.34 (m, 1H), 1.89-1.86 (m, 2H), 1.69-1.66 (m, 2H).

The following compounds were prepared following the procedure of I-119:

| Cmpd. | | |
|---|---|---|
| No. | Structure and Name | Analytical Data |
| I-130 | 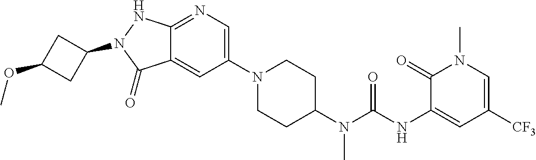 [SMILES: CO[C@H]1C[C@@H](n2[nH]c3ncc(N4CCC(N(C)C(=O)Nc5cc(C(F)(F)F)cn(C)c5=O)CC4)cc3c2=O)C1] 1-(1-(2-((1s,3s)-3-methoxycyclobutyl)-3- oxo-2,3-dihydro-1H-pyrazolo[3,4- b]pyridin-5-yl)piperidin-4-yl)-1-methyl- 3-(1-methyl-2-oxo-5-(trifluoromethyl)- 1,2-dihydropyridin-3-yl)urea | MS (ES): m/z 550.13 [M + H]⁺ ¹H NMR (DMSO-d6, 400 MHz): δ 10.34 (s, 1H), 8.47 (s, 1H), 8.16 (s, 1H), 8.03 (s, 1H), 7.90 (s, 1H), 7.53 (s, 1H), 4.58 (m, 1H), 4.17 (m, 1H), 3.69 (m, 3H), 3.58 (s, 3H), 3.17 (s, 3H), 2.88 (s, 3H), 2.82-2.73 (m, 3H), 2.42-2.31 (m, 3H), 1.92-1.80 (m, 2H), 1.61-1.69 (m, 2H) |

### Example 120: trans-1-(4-(5H-pyrrolo[2,3-b]pyrazin-2-yl)cyclohexyl)-1-methyl-3-(1-methyl-2-oxo-5-(trifluoromethyl)-1,2-dihydropyridin-3-yl)urea and cis-1-(4-(5H-pyrrolo[2,3-b]pyrazin-2-yl)cyclohexyl)-1-methyl-3-(1-methyl-2-oxo-5-(trifluoromethyl)-1,2-dihydropyridin-3-yl)urea

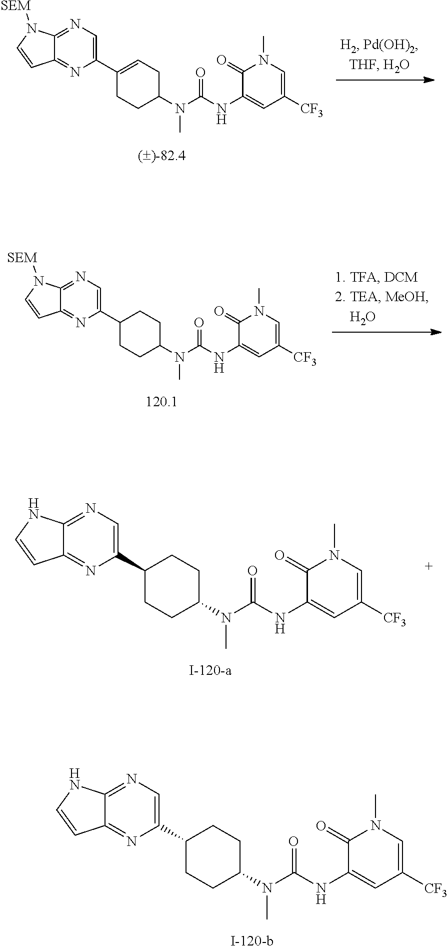 [SMILES: CN(C(=O)Nc1cc(C(F)(F)F)cn(C)c1=O)C1CC=C(c2cnc3c(ccn3COCC[Si](C)(C)C)n2)CC1 ; CN(C(=O)Nc1cc(C(F)(F)F)cn(C)c1=O)C1CCC(c2cnc3c(ccn3COCC[Si](C)(C)C)n2)CC1 ; Cn1cc(C(F)(F)F)cc(NC(=O)N(C)[C@H]2CC[C@H](c3cnc4[nH]ccc4n3)CC2)c1=O ; Cn1cc(C(F)(F)F)cc(NC(=O)N(C)[C@H]2CC[C@@H](c3cnc4[nH]ccc4n3)CC2)c1=O]

Synthesis of compound 120.1. A mixture of (±)-82.4 (0.155 g, 0.268 mmol, 1 equiv), palladium(II) hydroxide (0.078 g), THF (1 mL), water (1 mL) and methanol (0.7 mL) was stirred under hydrogen for 12 h. The reaction mixture was filtered through a pad of Celite® and rinsed with methanol. The filtrate was concentrated under reduced pressure. The residue was purified by flash column chromatography on silica gel (CombiFlash®, 5% methanol in DCM) to afford 120.1 (0.153 g, 98%). MS(ES): m/z 579.71 [M+H]⁺.

Synthesis of I-120-a and I-120-b. To a solution of 120.1 (0.153 g, 0.26438 mmol, 1.0 equiv) in DCM (2.61 mL) at 0° C. was added trifluoroacetic acid (2.61 mL). The reaction mixture was stirred at room temperature for 15 min. It was basified with saturated sodium bicarbonate solution and extracted with ethyl acetate. The combined organic layers were washed with brine, dried over anhydrous sodium sulfate, filtered, and concentrated under reduced pressure. To the residue was added methanol (2.61 mL), water (1.224 mL) and triethylamine (1.785 mL) at 0° C. The reaction mixture was stirred at room temperature overnight. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.2% methanol in DCM) to afford I-120-a (0.029 g, 24%) and I-120-b (0.032 g, 27%).

I-120-a: MS(ES): m/z: 449.45 [M+H]⁺, ¹H NMR (DMSO-d₆, 400 MHz): δ 11.89 (s, 1H), 8.17 (s, 2H), 8.02 (s, 1H), 7.91 (s, 1H), 7.80 (s, 1H), 6.56 (bs, 1H), 4.10 (m, 1H), 3.59 (s, 3H), 2.91 (s, 3H), 2.84 (m, 1H), 2.07-1.97 (m, 2H), 1.85-1.75 (m, 6H).

I-120-b: MS(ES): m/z: 449.45 [M+H]⁺, ¹H NMR (DMSO-d₆, 400 MHz): δ 11.90 (s, 1H), 8.29 (s, 1H), 8.16 (s, 1H), 8.01 (s, 1H), 7.81-7.80 (m, 2H), 6.60 (s, 1H), 4.14 (m, 1H), 3.57 (s, 3H), 3.27 (m, 1H), 2.72 (s, 3H), 2.44-2.41 (m, 2H), 1.89-1.80 (4H), 1.55-1.43 (m, 2H).

### Example 122: (S)-1-(3,3-difluoro-1-(3-(1-methylpiperidin-4-yl)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and (R)-1-(3,3-difluoro-1-(3-(1-methylpiperidin-4-yl)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

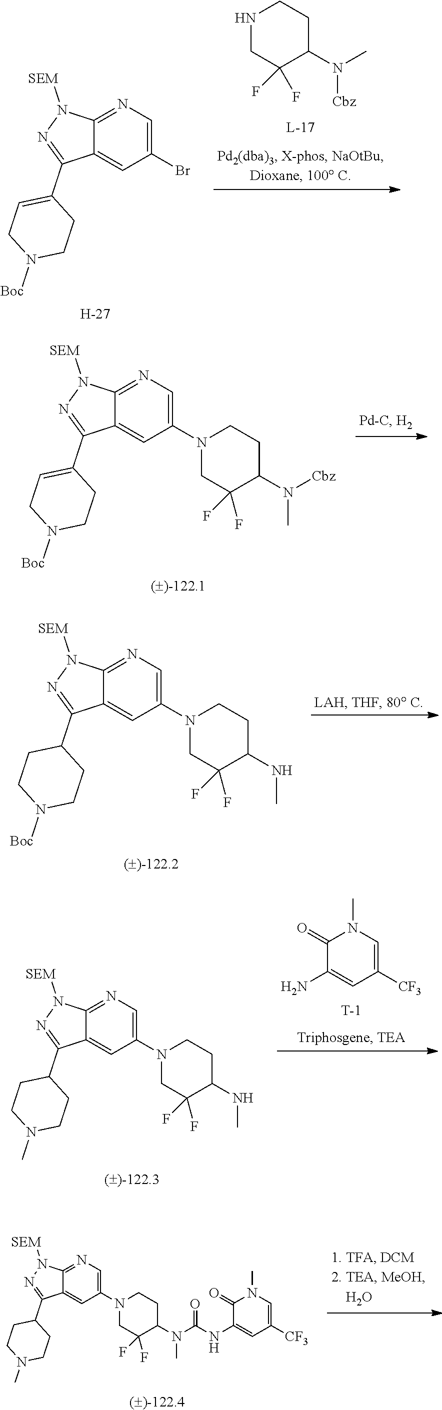

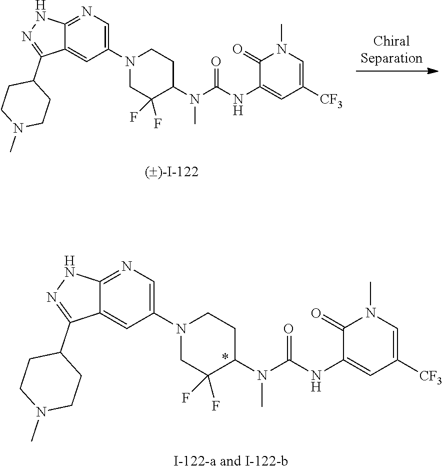

Synthesis of compound (±)-122.1. Compound (±)-122.1 was prepared from compound H-27 and L-17 following the procedure described in the synthesis of 11.1. The product was purified by flash column chromatography on silica gel (CombiFlash®, 70% ethyl acetate in hexane in). MS(ES): m/z: 713.36 [M+H]⁺.

Synthesis of compound (±)-122.2. A mixture of palladium on carbon (10 wt %, 0.2 g) and (±)-122.1 (1.0 g, 1.40 mmol, 1.0 equiv) in methanol (15 mL) was stirred under hydrogen atmosphere (1 atm) for 2 h. It was filtered through a pad of Celite® and rinsed with methanol. The combined filtrate was concentrated under reduced pressure to afford (±)-122.2 (0.73 g, 90%). MS(ES): m/z 581.34 [M+H]⁺.

Synthesis of compound (±)-122.3. To a solution of (±)-122.2 (0.30 g, 516.53 μmol, 1.0 equiv) in THF (3.0 mL) was added 1 M solution of lithium aluminum hydride in THF (3.6 mL, 0.362 mmol, 7.0 equiv) at 0° C. for 20 min. It was quenched by saturated solution of anhydrous sodium sulfate and extracted with ethyl acetate. The combined organic layers were washed with brine, dried over anhydrous sodium sulfate, filtered, and concentrated under reduced pressure. The residue was purified by flash column chromatography on silica gel (CombiFlash®, 3.6% methanol in DCM) to afford (±)-122.3 (0.240 g, 97%). MS(ES): m/z 495.30 [M+H]⁺.

Synthesis of compound (±)-122.4. Compound (±)-122.4 was prepared from compound (±)-122.3 and T-1 following the procedure described in the synthesis of I-9. The product was purified by flash column chromatography on silica gel (CombiFlash®, 3.6% methanol in DCM). MS(ES): m/z 713.33 [M+H]⁺.

Synthesis of compound (±)-I-122. Compound (±)-I-122 was prepared from compound (±)-122.4 following the procedure described in the synthesis of I-11. The product was purified by flash column chromatography on silica gel (CombiFlash®, 3.2% methanol in DCM). MS (ES): m/z 583.58 [M+H]⁺.

I-122-a and I-122-b. The racemate was separated by chiral SFC (Method D) to afford first eluting fraction (I-122-a) and second eluting fraction (I-122-b). (*Absolute configuration was not determined.)

I-122-a: MS(ES): m/z: 583.32 [M+H]⁺, ¹H NMR (DMSO-d₆, 400 MHz): δ 13.02 (s, 1H), 8.43 (s, 1H), 8.16 (s, 1H), 8.08 (s, 1H), 8.09 (s, 1H), 7.77 (s, 1H), 4.80-4.78 (m, 1H), 3.97-3.88 (m, 2H), 3.81-3.73 (m, 2H), 3.60 (s, 3H), 3.05 (s, 3H), 2.94-2.87 (m, 3H), 2.66-2.63 (m, 3H), 2.33-2.28 (m, 1H), 2.21 (s, 3H), 2.06-2.01 (m, 2H), 1.97-1.83 (m, 2H).

I-122-a: MS(ES): m/z: 583.19 [M+H]⁺, ¹H NMR (DMSO-d₆, 400 MHz): δ 13.02 (s, 1H), 8.43 (s, 1H), 8.16 (s, 1H), 8.08 (s, 1H), 8.09 (s, 1H), 7.77 (s, 1H), 4.80-4.78 (m, 1H), 3.97-3.87 (m, 2H), 3.81-3.72 (m, 2H), 3.60 (s, 3H), 3.06 (s, 3H), 2.94-2.88 (m, 3H), 2.66-2.63 (m, 3H), 2.33-2.27 (m, 1H), 2.21 (s, 3H), 2.06-2.01 (m, 2H), 1.97-1.82 (m, 2H).

### Example 123: (R)-1-(1-(3-(1-acetylpiperidin-4-yl)-1H-pyrazolo[3,4-b]pyridin-5-yl)-3,3-difluoropiperidin-4-yl)-1-methyl-3-(1-methyl-2-oxo-5-(trifluoromethyl)-1,2-dihydropyridin-3-yl)urea and (S)-1-(1-(3-(1-acetylpiperidin-4-yl)-1H-pyrazolo[3,4-b]pyridin-5-yl)-3,3-difluoropiperidin-4-yl)-1-methyl-3-(1-methyl-2-oxo-5-(trifluoromethyl)-1,2-dihydropyridin-3-yl)urea

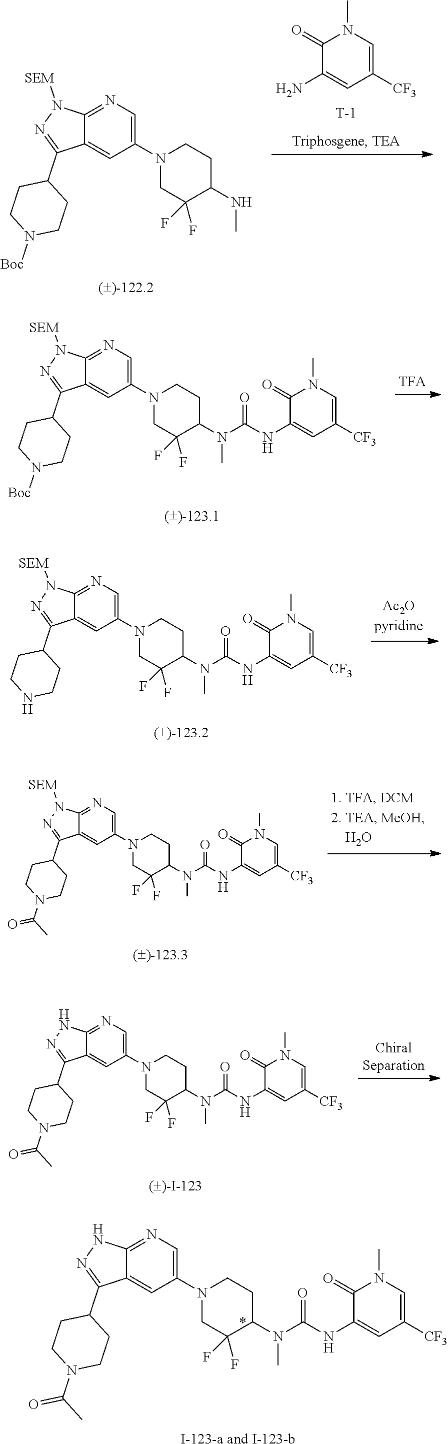

Synthesis of compound (±)-123.1. Compound (±)-123.1 was prepared from compound (±)-122.2 and T-1 following the procedure described in the synthesis of I-9. The product was purified by flash column chromatography on silica gel (CombiFlash®, 3.6% methanol in DCM). MS(ES): m/z 799.93 [M+H]⁺.

Synthesis of compound (±)-123.2. To a solution of (±)-123.1 (0.350 g, 0.438 mmol, 1.0 equiv) in DCM (5.0 mL) was added trifluoroacetic acid (2.0 mL) dropwise at 0° C. The reaction mixture was stirred at room temperature for 2 h. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 4.0% methanol in DCM) to afford (±)-123.2 (0.250 g, 82%). MS(ES): m/z 699.31 [M+H]⁺.

Synthesis of compound (±)-123.3. To a solution of (±)-123.2 (0.250 g, 0.358 mmol, 1.0 equiv) in DCM (5.0 mL) was added pyridine (0.1420 g, 1.79 mmol, 5.0 equiv) followed by acetic anhydride (0.110 g, 1.07 mmol, 3.0 equiv) and stirred at room temperature for 20 min. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 2.0% methanol in DCM) to afford (f)-123.3 (0.115 g, 43%). MS(ES): m/z 741.33 [M+H]⁺.

Synthesis of compound (±)-I-123. Compound (±)-I-123 was prepared from compound (±)-123.3 following the procedure described in the synthesis of I-11. The product was purified by flash column chromatography on silica gel (CombiFlash®, 6.2% methanol in DCM). MS(ES): m/z 611.59 [M+H]⁺.

I-123-a and I-123-b. The racemate was separated by chiral SFC (Method D) to afford first eluting fraction (I-123-a) and second eluting fraction (I-123-b). (*Absolute configuration was not determined.)

I-123-a: MS(ES): m/z: 612.2 [M+H]⁺, ¹H NMR (DMSO-d₆, 400 MHz): δ 13.07 (s, 1H), 8.44 (s, 1H), 8.16 (s, 1H), 8.08 (s, 1H), 8.07 (s, 1H), 7.80 (s, 1H), 4.48-4.45 (m, 1H), 3.93-3.80 (m, 2H), 3.77-3.73 (m, 1H), 3.60 (s, 3H), 3.25-3.21 (m, 2H), 3.18-3.05 (m, 1H), 3.09 (s, 3H), 2.95-2.31 (m, 2H), 2.37-2.27 (m, 2H), 2.04 (s, 3H), 2.00-1.97 (m, 2H), 1.87-1.81 (m, 1H), 1.80-1.77 (m, 1H), 1.63-1.61 (m, 1H).

I-123-b: MS(ES): m/z: 612.2 [M+H]⁺, ¹H NMR (DMSO-d₆, 400 MHz): δ 13.07 (s, 1H), 8.44 (s, 1H), 8.16 (s, 1H), 8.08 (s, 1H), 8.07 (s, 1H), 7.80 (s, 1H), 4.48-4.45 (m, 1H), 3.93-3.80 (m, 2H), 3.77-3.73 (m, 1H), 3.60 (s, 3H), 3.25-3.21 (m, 2H), 3.18-3.05 (m, 1H), 3.05 (s, 3H), 2.95-2.31 (m, 2H), 2.37-2.27 (m, 2H), 2.04 (s, 3H), 2.00-1.97 (m, 2H), 1.88-1.81 (m, 1H), 1.80-1.76 (m, 1H), 1.63-1.60 (m, 1H).

### Example 125: (R)-1-(3,3-difluoro-1-(3-(methylamino)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and (S)-1-(3,3-difluoro-1-(3-(methylamino)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

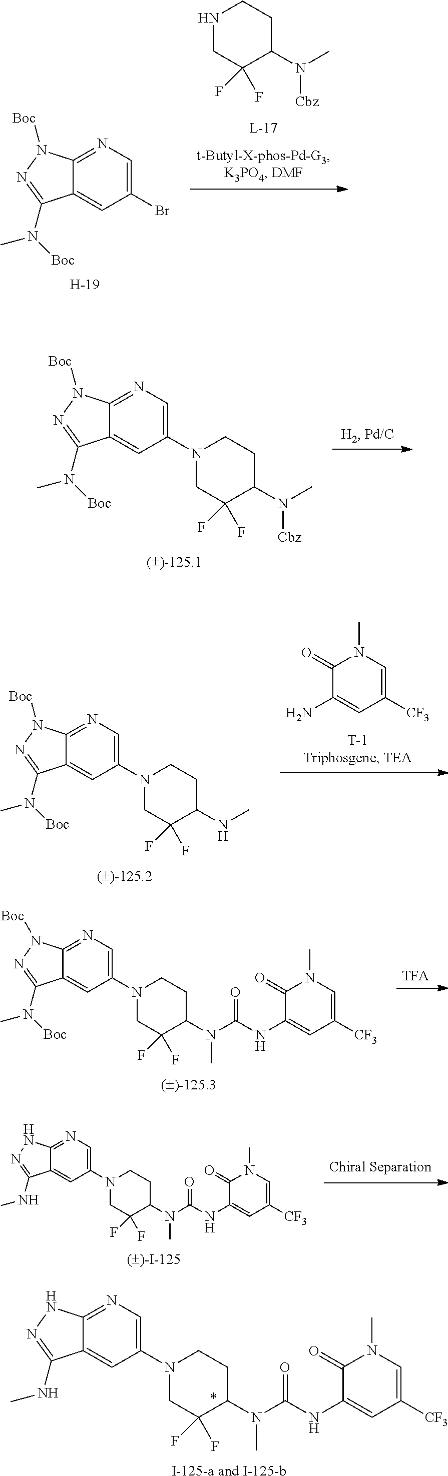

Synthesis of compound (±)-125.1. A mixture of H-19 (0.7 g, 2.462 mmol, 1.0 equiv), L-17 (0.84 g, 2.958 mmol, 1.2 equiv), and potassium phosphate (1.567 g, 7.394 mmol, 3.0 equiv) in DMF (7 mL) was degassed by bubbling through a stream of argon under argon atmosphere for 15 min. Methanesulfonato(2-di-t-butylphosphino-2′,4′,6′-tri-1-propyl-1,1′-biphenyl) (2′-amino-1,1′-biphenyl-2-yl)palladium(II) (0.097 g, 0.1232 mmol, 0.05 equiv) was added and stirred at room temperature for 12 h. It was filtered through a pad of Celite® and filtrate was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford (±)-125.1 (0.32 g, 21%). MS(ES): m/z 631.69 [M+H]⁺.

Synthesis of compound (±)-125.2. A mixture of compound (±)-125.1 (0.32 g, 0.507 mmol, 1.0 equiv), methanol (3.2 mL), and palladium on carbon (10 wt %, 0.1 g) was stirred under hydrogen (1 atm) for 2 h. The reaction mixture was filtered through a pad of Celite® and rinsed with methanol. The filtrate was concentrated under reduced pressure. Then the residue was purified by flash column chromatography on silica gel (CombiFlash®, 5% methanol in DCM) to afford (±)-125.2 (0.265 g, 95%). MS(ES): m/z 497.56 [M+H]⁺.

Synthesis of compound (±)-125.3. Compound (±)-125.3 was prepared from compound (±)-125.2 and T-1 following the procedure described in the synthesis of I-9. The product was purified by flash column chromatography on silica gel (3.7% methanol in DCM). MS(ES): m/z 715.70 [M+H]⁺.

Synthesis of compound (±)-125. To a solution of (±)-125.3 (0.147 g, 0.2056 mmol, 1.0 equiv) in DCM (0.88 mL) at 0° C. was added trifluoroacetic acid (0.88 mL) under nitrogen atmosphere. The mixture was stirred at room temperature for 15 min. It was quenched with water, basified with saturated sodium bicarbonate solution, and extracted with ethyl acetate. The combined organic layers were washed with brine, dried over anhydrous sodium sulfate, filtered, and concentrated under reduced pressure to afford (±)-125 (0.081 g, 77%) MS(ES): m/z 515.46 [M+H]⁺.

I-125-a and I-125-b. The racemate was separated with chiral SFC (Method H) to afford first eluting fraction (I-125-a) and second eluting fraction (I-125-a).

I-125-a: MS(ES): m/z: 515.46 [M+H]⁺, ¹H NMR (DMSO-d₆, 400 MHz): δ 11.76 (s, 1H), 8.31 (s, 1H), 8.15 (s, 1H), 8.07 (s, 2H), 7.71 (s, 1H), 5.97-5.96 (m, 1H), 4.89-4.72 (m, 1H), 3.82-3.76 (m, 1H), 3.69-3.53 (m, 3H), 3.06 (s, 6H), 2.89 (s, 3H), 2.42-2.21 (m, 1H), 1.91-1.82 (m, 1H).

I-125-b: MS(ES): m/z 515.46 [M+H]⁺, ¹H NMR (DMSO-d₆, 400 MHz): δ 11.78 (s, 1H), 8.33 (s, 1H), 8.17 (s, 1H), 7.73 (s, 2H), 5.98-5.97 (m, 1H), 5.98-5.97 (m, 1H), 4.90-4.75 (m, 1H), 3.80-3.71 (m, 1H), 3.62-3.69 (m, 1H), 3.13-3.29 (m, 2H), 3.25 (s, 6H), 3.04 (s, 3H), 2.34-3.29 (m, 1H), 1.90-1.87 (m, 1H).

### Example 129: 1-(1-(6-((1R,2R)-2-hydroxycyclobutoxy)pyrazolo[1,5-a]pyrazin-3-yl)piperidin-4-yl)-1-methyl-3-(1-methyl-2-oxo-5-(trifluoromethyl)-1,2-dihydropyridin-3-yl)urea and 1-(1-(6-((1S,2S)-2-hydroxycyclobutoxy)pyrazolo[1,5-a]pyrazin-3-yl)piperidin-4-yl)-1-methyl-3-(1-methyl-2-oxo-5-(trifluoromethyl)-1,2-dihydropyridin-3-yl)urea

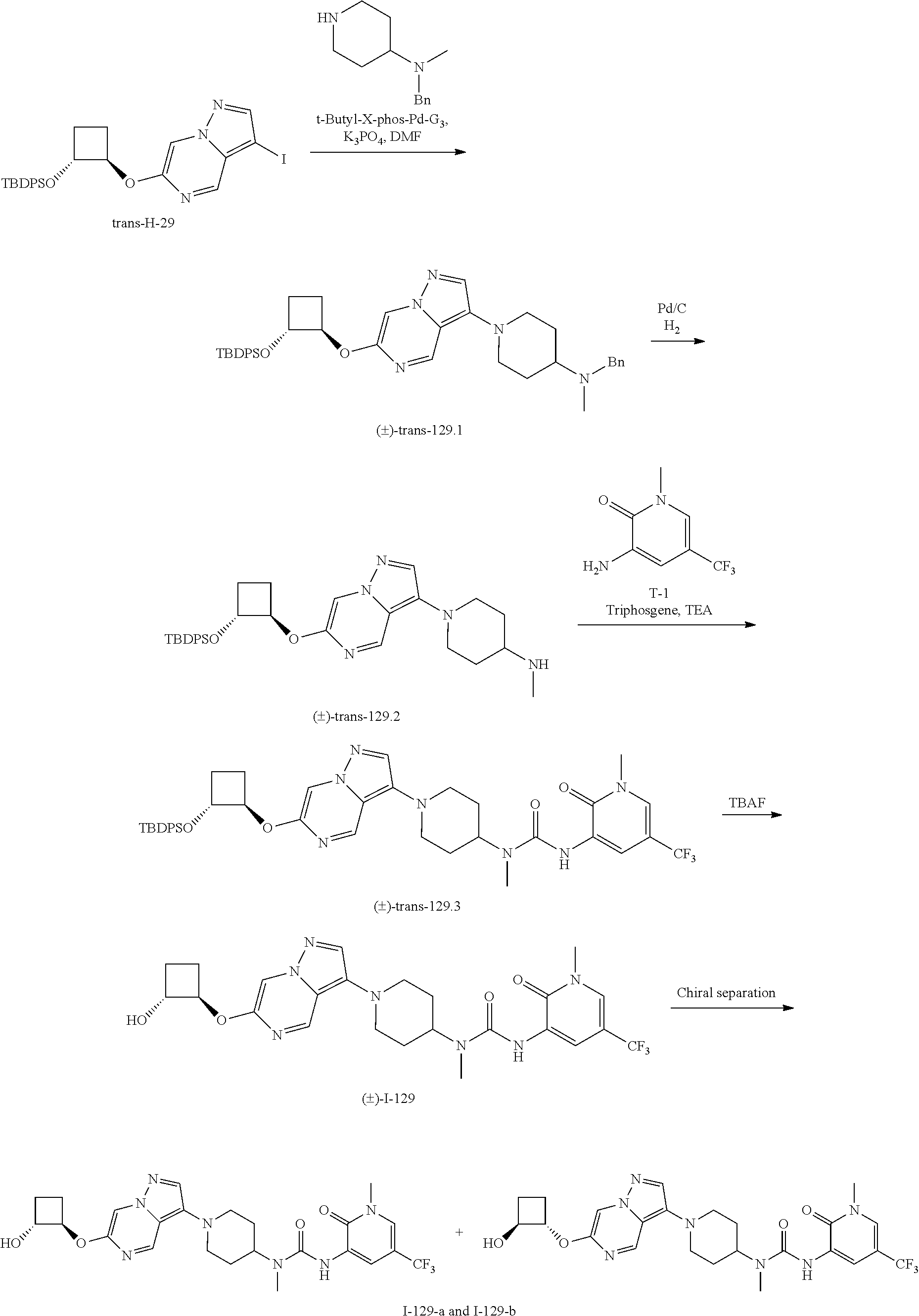

Synthesis of compound (±)-trans-129.1. A mixture of trans-H-29 (0.540 g, 0.947 mmol, 1.0 equiv), N-benzyl-N-methylpiperidin-4-amine (0.213 g, 1.04 mmol, 1.1 equiv) and potassium phosphate (0.602 g, 2.84 mmol, 3.0 equiv) in DMF (3.0 mL) was degassed by bubbling through a stream of argon for 10 min. [(2-Di-tert-butylphosphino-2′,4′,6′-triisopropyl-1,1′-biphenyl)-2-(2′-amino-1,1′-biphenyl)]palladium(II) methanesulfonate (0.242 g, 0.284 mmol, 0.3 equiv) was added and degassed for 5 min. The reaction mixture was stirred at room temperature for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford (±)-trans-129.1 (0.26 g, 43%). MS(ES): m/z 646.35 [M+H]⁺.

Synthesis of compound (±)-trans-129.2. A mixture of compound (±)-trans-129.1 (0.26 g, 0.40 mmol, 1.0 equiv), THE (2.0 mL) and palladium on carbon (10 wt %, 0.1 g) was stirred under hydrogen (1 atm) for 6 h. The reaction mixture was filtered through a pad of Celite® and rinsed with methanol. The filtrate was concentrated under reduced pressure to afford (±)-trans-129.2 (0.200 g, 90%). MS(ES): m/z 556.30 [M+H]⁺.

Synthesis compound (±)-trans-129.3. Compound (±)-trans-129.3 was prepared from compound (±)-trans-129.2 and T-1 following the procedure described in the synthesis of I-9. The product was purified by flash column chromatography on silica gel (2.5% methanol in DCM)). MS(ES): m/z: 774.33 [M+H]⁺.

Synthesis of compound (±)-I-129. To a solution of (±)-trans-129.3 (0.105 g, 0.13 mmol, 1.0 equiv) in THE (10 mL) was added tetrabutylammonium fluoride solution (1 M in THF, 0.16 mL, 0.16 mmol, 1.2 equiv) at 0° C. and stirred for 1 h. It was transferred into ice-water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.0% methanol in DCM) to afford (±)-I-129 (0.065 g, 93%). MS (ES): m/z 536.22 [M+H]⁺.

I-129-a and I-129-b: The racemate was separated by chiral HPLC (Method L) to afford first eluting fraction (I-129-a) and second eluting fraction (I-129-b).

I-129-a: MS(ES): m/z: 536.16 [M+H]⁺, ¹H-NMR (400 MHz, DMSO-d₆): δ 8.96 (s, 1H), 8.19 (s, 1H), 8.18 (s, 1H), 8.04 (s, 1H), 7.92 (s, 1H), 7.71 (s, 1H), 5.69-5.68 (d, J=6.4 Hz, 1H), 4.56-4.54 (m, 1H), 4.21-4.14 (m, 1H), 4.07-4.06 (m, 1H), 3.59 (s, 3H), 2.91 (s, 3H), 2.85-2.79 (m, 2H), 2.20-2.17 (m, 1H), 2.15-1.96 (m, 3H), 1.68-1.65 (m, 2H), 1.47-1.39 (m, 2H), 1.17-1.14 (m, 2H).

I-129-b: MS(ES): m/z 536.18 [M+H]⁺, ¹H-NMR (400 MHz, DMSO-d₆): δ 8.96 (s, 1H), 8.20 (s, 1H), 8.17 (s, 1H), 8.04 (s, 1H), 7.92 (s, 1H), 7.71 (s, 1H), 5.69-5.68 (d, J=6.4 Hz, 1H), 4.56-4.54 (m, 1H), 4.21-4.14 (m, 1H), 4.07-4.06 (m, 1H), 3.59 (s, 3H), 2.91 (s, 3H), 2.84-2.79 (m, 2H), 2.17-2.16 (m, 1H), 2.07-1.95 (m, 3H), 1.68-1.65 (m, 2H), 1.47-1.39 (m, 2H), 1.17-1.14 (m, 2H).

### Example 134: N-(5-((2S,4S)-2-methyl-4-(1-methyl-3-(1-methyl-2-oxo-5-(trifluoromethyl)-1,2-dihydropyridin-3-yl)ureido)piperidin-1-yl)pyrazin-2-yl)acetamide and N-(5-((2R,4R)-2-methyl-4-(1-methyl-3-(1-methyl-2-oxo-5-(trifluoromethyl)-1,2-dihydropyridin-3-yl)ureido)piperidin-1-yl)pyrazin-2-yl)acetamide

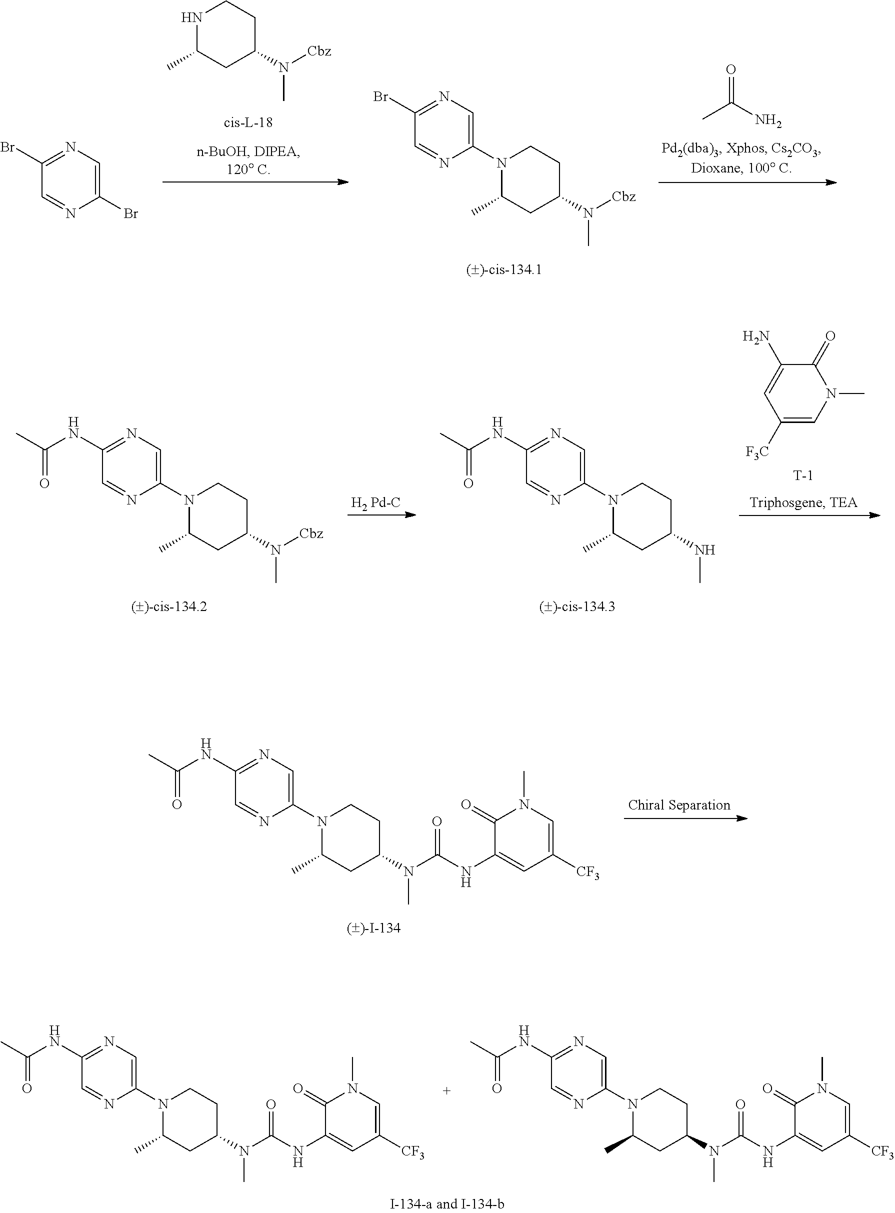

Synthesis of compound (±)-cis-134.1. A solution of 2,5-dibromopyrazine (1.0 g, 4.20 mmol, 1.0 equiv), N,N′-diisopropylethylaamine (2.72 g, 21.02 mmol, 5 equiv) and cis-L-18 (2.2 g, 8.41 mmol, 2 equiv) in 1-butanol (15.0 mL) was stirred at 120° C. for 16 h. It was filtered through a pad of Celite® and rinsed with ethyl acetate. The filtrate was washed with water and extracted with ethyl acetate. The organic layer was dried over anhydrous sodium sulfate, filtered, and concentrated under reduced pressure. The residue was purified by flash column chromatography on silica gel (CombiFlash®, 30% ethyl acetate in hexane) to afford (±)-cis-134.1 (0.35 g, 20%). MS(ES): m/z 420.32 [M+H]⁺.

Synthesis of compound (±)-cis-134.2. A mixture of (±)-cis-134.1 (0.35 g, 834.68 mmol, 1.0 equiv), acetamide (0.148 g, 2.50 mmol, 5.0 equiv) and cesium carbonate (0.815 g, 2.50 mmol, 3.0 equiv) in 1,4-dioxane (5.0 mL) was degassed by bubbling through a stream of argon for 10 min. 2-Dicyclohexylphosphino-2′,4′,6′-triisopropylbiphenyl (0.08 g, 166.94 μmol, 0.2 equiv) and tris(dibenzylideneacetone)dipalladium(O) (0.076 g, 0.0834 mmol, 0.1 equiv) were added and degassed for 15 min. The reaction mixture was stirred at 100° C. for 1.5 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 70% ethyl acetate in hexane in) to afford (±)-cis-134.2 (0.25 g, 75%). MS(ES): m/z: 398.48 [M+H]⁺.

Synthesis of compound (±)-cis-134.3. A mixture of palladium on carbon (10 wt %, 0.2 g) and (±)-cis-134.2 (0.25 g, 628.96 μmol, 1.0 equiv) in methanol was stirred at rt under hydrogen atmosphere for 2 h. It was filtered through a pad of Celite® pad and rinsed with methanol. Combined filtrate was concentrated under reduced pressure to afford (±)-cis-134.3 (0.154 g, 93%). MS(ES): m/z 264.35 [M+H]⁺.

Synthesis of compound (±)-I-134. Compound (±)-I-134 was prepared from compound (±)-cis-134.3 and T-1 following the procedure described in the synthesis of I-9. The product was purified by flash column chromatography on silica gel (CombiFlash®, 3.6% methanol in DCM). MS(ES): m/z 482.48 [M+H]⁺.

I-134-a and I-134-b. The racemate was separated by chiral HPLC (Method G) to get afford first eluting fraction (I-134-a) and second eluting fraction (I-134-b).

I-134-a: MS(ES): m/z: 482.45 [M+H]⁺, ¹H NMR (DMSO-d₆, 400 MHz): δ 10.33 (s, 1H), 8.80 (s, 1H), 8.13 (s, 1H), 8.01 (s, 1H), 7.99 (s, 1H), 7.87 (s, 1H), 4.17-4.16 (m, 1H), 3.87-3.83 (m, 1H), 3.72-3.68 (m, 1H), 3.57 (s, 3H), 3.40-3.36 (m, 1H), 2.90 (s, 3H), 2.05 (s, 3H), 1.89-1.67 (m, 4H), 1.15 (s, 3H).

I-134-b: MS(ES): m/z: 482.25 [M+H]⁺, ¹H NMR (DMSO-d₆, 400 MHz): δ 10.33 (s, 1H), 8.80 (s, 1H), 8.13 (s, 1H), 8.01 (s, 1H), 7.99 (s, 1H), 7.87 (s, 1H), 4.17-4.16 (m, 1H), 3.87-3.83 (m, 1H), 3.72-3.68 (m, 1H), 3.57 (s, 3H), 3.40-3.36 (m, 1H), 2.90 (s, 3H), 2.05 (s, 3H), 1.89-1.67 (m, 4H), 1.15 (s, 3H).

### Example 135: 3-(5-(tert-butyl)-1-((1r,3R)-3-hydroxycyclobutyl)-1H-pyrazol-3-yl)-1-methyl-1-((2R,4R)-2-methyl-1-(1H-pyrazolo[3,4-b]pyridin-5-yl)piperidin-4-yl)urea and 3-(5-(tert-butyl)-1-((1r,3S)-3-hydroxycyclobutyl)-1H-pyrazol-3-yl)-1-methyl-1-((2S,4S)-2-methyl-1-(1H-pyrazolo[3,4-b]pyridin-5-yl)piperidin-4-yl)urea

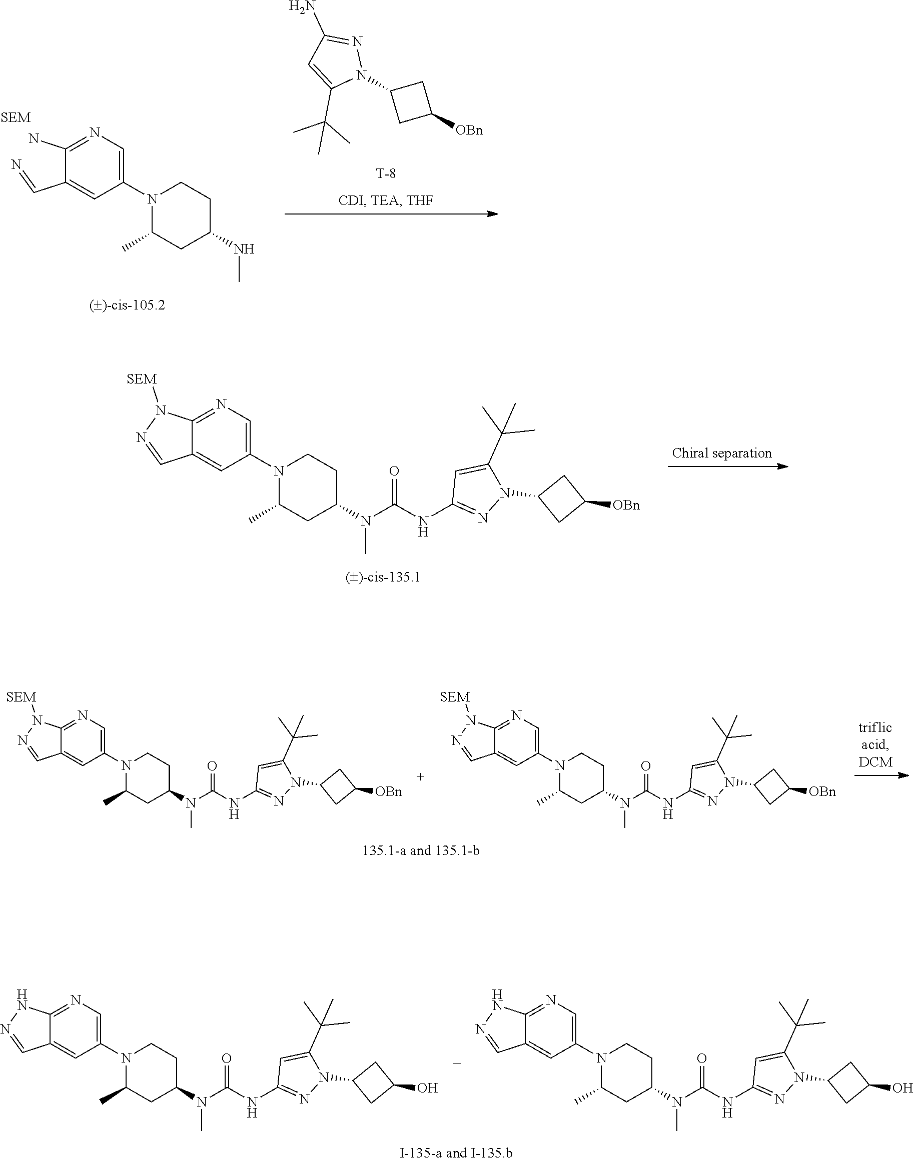

Synthesis of compound (±)-cis-135.1. To a solution of (±)-cis-105.2 (0.2 g, 0.533 mmol, 1.0 equiv) and T-8 (0.175 g, 0.586 mmol, 1.1 equiv) in THE (3.0 mL) at 0° C. was added triethylamine (0.193 mL, 1.333 mmol, 2.5 equiv) followed by 1,1′-carbonyldiimidazole (0.13 g, 0.80 mmol, 1.5 equiv). The reaction mixture was stirred at room temperature for 6 h. It was poured into ice water and extracted with ethyl acetate. The combined organic layers were washed with brine, dried over anhydrous sodium sulfate, filtered, and concentrated under reduced pressure. The residue was purified by column chromatography on silica gel (1.5% methanol in DCM) to afford (±)-cis-135.1 (0.15 g, 37%). MS(ES): m/z 701.1 [M+H]⁺.

135.1-a and 135.1-b: The racemate was separated by chiral SFC (Method J) to afford first eluting fraction (135.1-a), MS(ES): m/z: 701.66 [M+H]⁺; and second eluting fraction (135.1-b), MS(ES): m/z 701.65 [M+H]⁺.

Synthesis of compound I-135-a. To a solution of 135.1-a (0.06 g, 0.085 mmol, 1.0 equiv) in DCM (2.0 mL) at 0° C. was added trifluoromethanesulfonic acid (0.6 mL). The reaction mixture was stirred at room temperature for 30 min. It was concentrated under reduced pressure. The residue was diluted with DCM (2 mL), basified with saturated sodium bicarbonate solution, and extracted with DCM. The combined organic layers were washed with brine, dried over anhydrous sodium sulfate, filtered, and concentrated under reduced pressure. To the residue was added water (1.0 mL), triethylamine (2.0 mL) and methanol (2.0 mL) stirred for 16 h at room temperature. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2.5% methanol in DCM) to afford I-135-a (0.019 g, 46%). MS(ES): m/z 481.44 [M+H]⁺, ¹H-NMR (400 MHz, DMSO-d₆): δ 13.55 (s, 1H), 8.66 (s, 1H), 8.34 (s, 1H), 8.06 (s, 1H), 8.02-8.01 (d, J=2.4 Hz, 1H), 6.13 (s, 1H), 5.18-5.11 (m, 2H), 4.42-4.40 (m, 1H), 4.27-4.26 (m, 1H), 3.33 (s, 3H), 3.13-3.07 (m, 2H), 2.92-2.91 (m, 1H), 2.83 (s, 3H), 2.71-2.64 (m, 2H), 2.30-2.24 (m, 2H), 1.88-1.85 (m, 1H), 1.67-1.55 (m, 3H), 1.29 (s, 9H).

Synthesis of compound 1-135-b. Compound 1-135-b was prepared from 135.1-b following the synthesis of 1-135-a. The product was purified by flash column chromatography on silica gel (CombiFlash®, 2.5% methanol in DCM). MS(ES): m/z 481.42 [M+H]⁺, ¹H-NMR (400 MHz, DMSO-d₆): δ 13.55 (s, 1H), 8.66 (s, 1H), 8.34 (s, 1H), 8.07 (s, 1H), 8.01 (s, 1H), 6.13 (s, 1H), 5.16-5.12 (m, 2H), 4.42-4.40 (m, 1H), 4.27-4.26 (m, 1H), 3.33 (s, 3H), 3.11-3.10 (m, 2H), 3.11-3.10 (m, 1H), 2.84 (s, 3H), 2.71-2.64 (m, 2H), 2.30-2.24 (m, 2H), 1.99-1.96 (m, 1H), 1.68-1.54 (m, 3H), 1.29 (s, 9H).

### Example 136: (S)-3-(5-cyclopropyl-2-oxo-1-(tetrahydro-2H-pyran-3-yl)-1,2-dihydropyridin-3-yl)-1-(1-(3-methoxy-1H-pyrazolo[3,4-b]pyridin-5-yl)piperidin-4-yl)-1-methylurea and (R)-3-(5-cyclopropyl-2-oxo-1-(tetrahydro-2H-pyran-3-yl)-1,2-dihydropyridin-3-yl)-1-(1-(3-methoxy-1H-pyrazolo[3,4-b]pyridin-5-yl)piperidin-4-yl)-1-methylurea

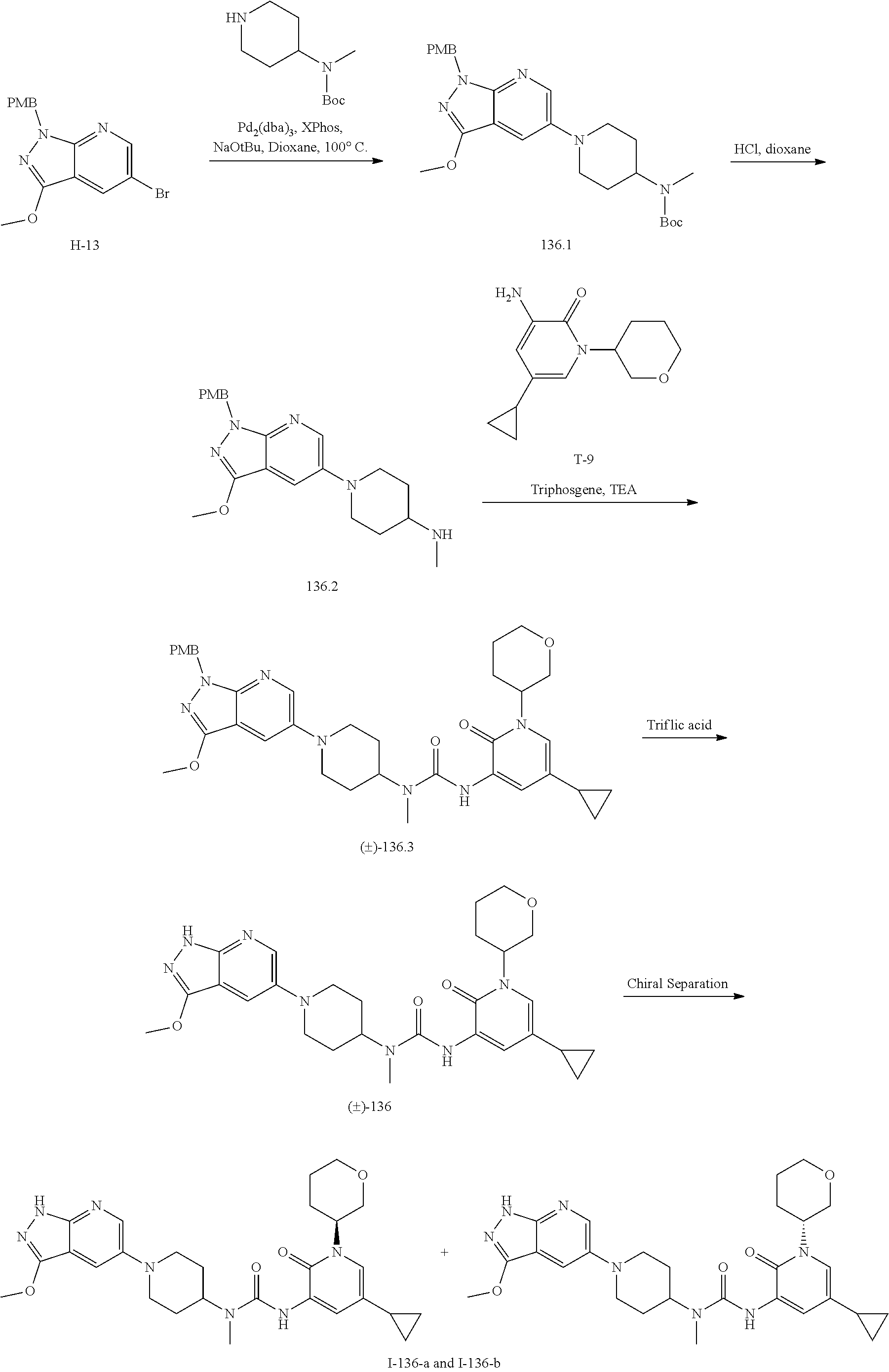

Synthesis of compound 136.1. Compound 136.1 was prepared from compound H-13 and tert-butyl methyl(piperidin-4-yl)carbamate following the procedure described in the synthesis of 11.1. The product was purified by flash column chromatography on silica gel (CombiFlash®, 20% ethyl acetate in hexane). MS(ES): m/z 482.60 [M+H]⁺.

Synthesis of compound 136.2. To a solution of 136.1 (0.26 g, 2.39 mmol, 1.0 equiv) in 1,4-dioxane (3 mL) was added 4 M hydrochloric acid in 1,4-dioxane (3 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure. The residue was purified by flash column chromatography on silica gel (CombiFlash®, 85% ethyl acetate in hexane) to afford 136.2 (0.180 g, 90%). MS(ES): m/z 382.48 [M+H]⁺.

Synthesis of compound (±)-136.3. Compound (±)-136.3 was prepared from compound 136.2 and T-9 following the procedure described in the synthesis of I-9. The product was purified by flash column chromatography on silica gel (CombiFlash®, 3.6% methanol in DCM). MS(ES): m/z 642.64 [M+H]⁺.

Synthesis of compound (±)-136. To a solution of (±)-136.3 (0.150 g, 3.70 mmol, 1.0 equiv) in DCM (2 mL) was added trifluoromethanesulfonic acid (0.5 mL) at 0° C. The reaction mixture was stirred at same temperature for 1 h. It was poured over ice water and basified with saturated sodium bicarbonate solution. The aqueous layer was extracted with ethyl acetate. The combined organic layers were washed with brine, dried over anhydrous sodium sulfate, filtered, and concentrated under reduced pressure material. The residue was purified by flash column chromatography on silica gel (CombiFlash®, 3% methanol in DCM) to afford (±)-136 (0.090 g, 74%). MS(ES): m/z 522.52 [M+H]⁺.

I-136-a and I-136-b: The racemate was separated by chiral HPLC (Method E) to afford eluting fraction (I-136-a) and second eluting fraction (I-136-b).

I-136-a: MS(ES): m/z: 522.40 [M+H]⁺, ¹H-NMR (400 MHz, DMSO-d₆): δ 12.27 (s, 1H), 8.42-8.41 (d, J=2.4 Hz, 1H), 7.86 (s, 1H), 7.75-7.74 (d, J=2.4 Hz, 1H), 7.47-7.46 (d, J=2.4 Hz, 1H), 7.25-7.24 (d, J=2.0 Hz, 1H), 4.82-4.77 (m, 1H), 4.19-4.12 (m, 1H), 3.98 (s, 3H), 3.83-3.3.705 (m, 2H), 3.66-3.57 (m, 3H), 3.47-3.42 (m, 1H), 2.87 (s, 3H), 2.78-2.73 (m, 2H), 2.10-2.06 (m, 1H), 1.94-1.86 (m, 3H), 1.80-1.75 (m, 2H), 1.67-1.65 (m, 3H), 0.85-0.80 (m, 2H), 0.54-0.50 (m, 2H).

I-136-b: MS(ES): m/z 522.38 [M+H]⁺, ¹H-NMR (400 MHz, DMSO-d₆): δ 12.27 (s, 1H), 8.42-8.41 (d, J=2.4 Hz, 1H), 7.86 (s, 1H), 7.75-7.74 (d, J=2.4 Hz, 1H), 7.47-7.46 (d, J=2.4 Hz, 1H), 7.25-7.24 (d, J=2.0 Hz, 1H), 4.82-4.77 (m, 1H), 4.19-4.13 (m, 1H), 3.98 (s, 3H), 3.83-3.3.75 (m, 2H), 3.66-3.57 (m, 3H), 3.47-3.42 (m, 1H), 2.87 (s, 3H), 2.78-2.73 (m, 2H), 2.10-2.03 (m, 1H), 1.97-1.86 (m, 3H), 1.80-1.79 (m, 2H), 1.67-1.64 (m, 3H), 0.85-0.80 (m, 2H), 0.54-0.50 (m, 2H).

The following compounds were prepared following the procedure of 1-136:

| Cmpd. | | |
|---|---|---|
| No. | Structure and Name | Analytical Data |
| I-138 | 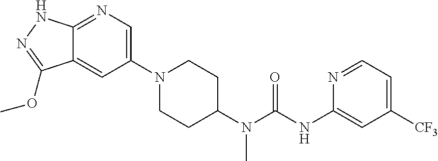 [SMILES: COc1n[nH]c2ncc(N3CCC(N(C)C(=O)Nc4cc(C(F)(F)F)ccn4)CC3)cc12] 1-(1-(3-methoxy-1H-pyrazolo[3,4- b]pyridin-5-yl)piperidin-4-yl)-1- methyl-3-(4- (trifluoromethyl)pyridin-2-yl)urea | MS (ES): m/z 450.44 [M + H]⁺ ¹H NMR (DMSO-d₆, 400 MHz): δ 12.26 (s, 1H), 9.46 (s, 1H), 8.51-8.50 (d, J = 4.8 Hz, 1H), 8.42-8.41 (d, J = 2.4 Hz, 1H), 8.18 (s, 1H), 7.46-7.45 (d, J = 2.4 Hz, 1H), 7.31-7.30 (d, J = 5.2 Hz, 1H), 4.25 -4.19 (s, 1H), 3.97 (s, 3H), 3.64-3.61 (m, 2H), 2.90 (s, 3H), 2.80- 2.74 (m, 2 H), 1.97-1.87 (m, 2H), 1.70-1.68 (m, 2H). |
| I-139 | 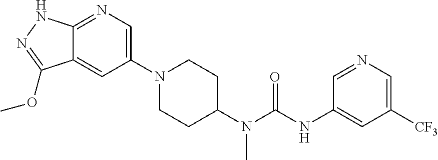 [SMILES: COc1n[nH]c2ncc(N3CCC(N(C)C(=O)Nc4cncc(C(F)(F)F)c4)CC3)cc12] 1-(1-(3-methoxy-1H-pyrazolo[3,4- b]pyridin-5-yl)piperidin-4-yl)-1- methyl-3-(5- (trifluoromethyl)pyridin-3-yl)urea | MS (ES): m/z 450.49 [M + H]⁺ ¹H NMR (DMSO-d₆, 400 MHz): δ 12.29 (s, 1H), 8.99 (s, 1H), 8.91 (s, 1H), 8.53 (s, 1H), 8.43 (s, 1H), 8.37 (s, 1H), 7.48 (s, 1H), 4.25- 4.19 (m, 1H), 3.99 (s, 3H), 3.67-3.64 (m, 2H), 2.92 (s, 3H), 2.82-2.76 (m, 2H), 1.95-1.92 (m, 2H), 1.72-1.69 (m, 2H). |

### Example 137: N-(4-methoxy-5-(4-(1-methyl-3-(1-methyl-2-oxo-5-(trifluoromethyl)-1,2-dihydropyridin-3-yl)ureido)piperidin-1-yl)pyridin-2-yl)cyclopropanecarboxamide

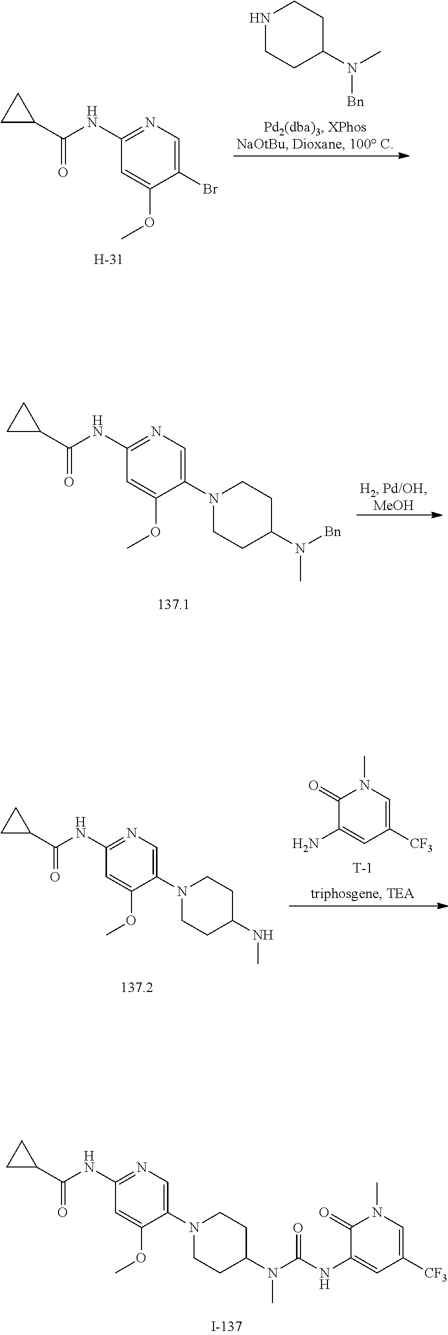

Synthesis of compound 137.1. Compound 137.1 was prepared from compound H-31 and N-benzyl-N-methylpiperidin-4-amine following the procedure described in the synthesis of 11.1. The product was purified by flash column chromatography on silica gel (CombiFlash®, 20% ethyl acetate in hexane). MS(ES): m/z 395.52 [M+H]⁺.

Synthesis of compound 137.2. A mixture of compound 137.1 (0.40 g, 1.015 mmol, 1.0 equiv), methanol (5 mL) and palladium on carbon (10 wt %, 0.1 g) was stirred under hydrogen for 2 h. The reaction mixture was filtered through a pad of Celite® and rinsed with methanol. The filtrate was concentrated under reduced pressure. The residue was purified by flash column chromatography on silica gel (CombiFlash®, 5% methanol in DCM) to afford 137.2 (0.145 g, 47%). MS(ES): m/z 305.39 [M+H]⁺.

Synthesis of compound I-137. Compound I-137 was prepared from compound 137.2 and T-1 following the procedure described in the synthesis of I-9. The product was purified by flash column chromatography on silica gel (CombiFlash®, 2.5% methanol in DCM. MS(ES): m/z 523.53 [M+H], ¹H NMR (DMSO-d₆, 400 MHz): δ 10.62 (s, 1H), 8.17-8.16 (d, J 2.4 Hz, 1H), 8.03 (s, 1H), 7.90 (s, 1H), 7.79-7.78 (d, J 6.0 Hz, 1H), 7.74 (s, 1H), 4.14 (m, 1H), 3.81 (s, 3H), 3.58 (s, 3H), 3.41-3.39 (m, 2H), 2.90 (s, 3H), 2.71-2.65 (m, 2H), 1.98-1.96 (m, 1H), 1.90-1.83 (m, 1H), 1.57-1.54 (m, 1H), 1.28-1.26 (m, 1H), 0.85-0.78 (m, 4H).

### Example 140: (S)—N-(5-(4-(1-methyl-3-(1-methyl-2-oxo-5-(trifluoromethyl)-1,2-dihydropyridin-3-yl)ureido)cyclohex-1-en-1-yl)pyrazin-2-yl)acetamide and (R)—N-(5-(4-(1-methyl-3-(1-methyl-2-oxo-5-(trifluoromethyl)-1,2-dihydropyridin-3-yl)ureido)cyclohex-1-en-1-yl)pyrazin-2-yl)acetamide

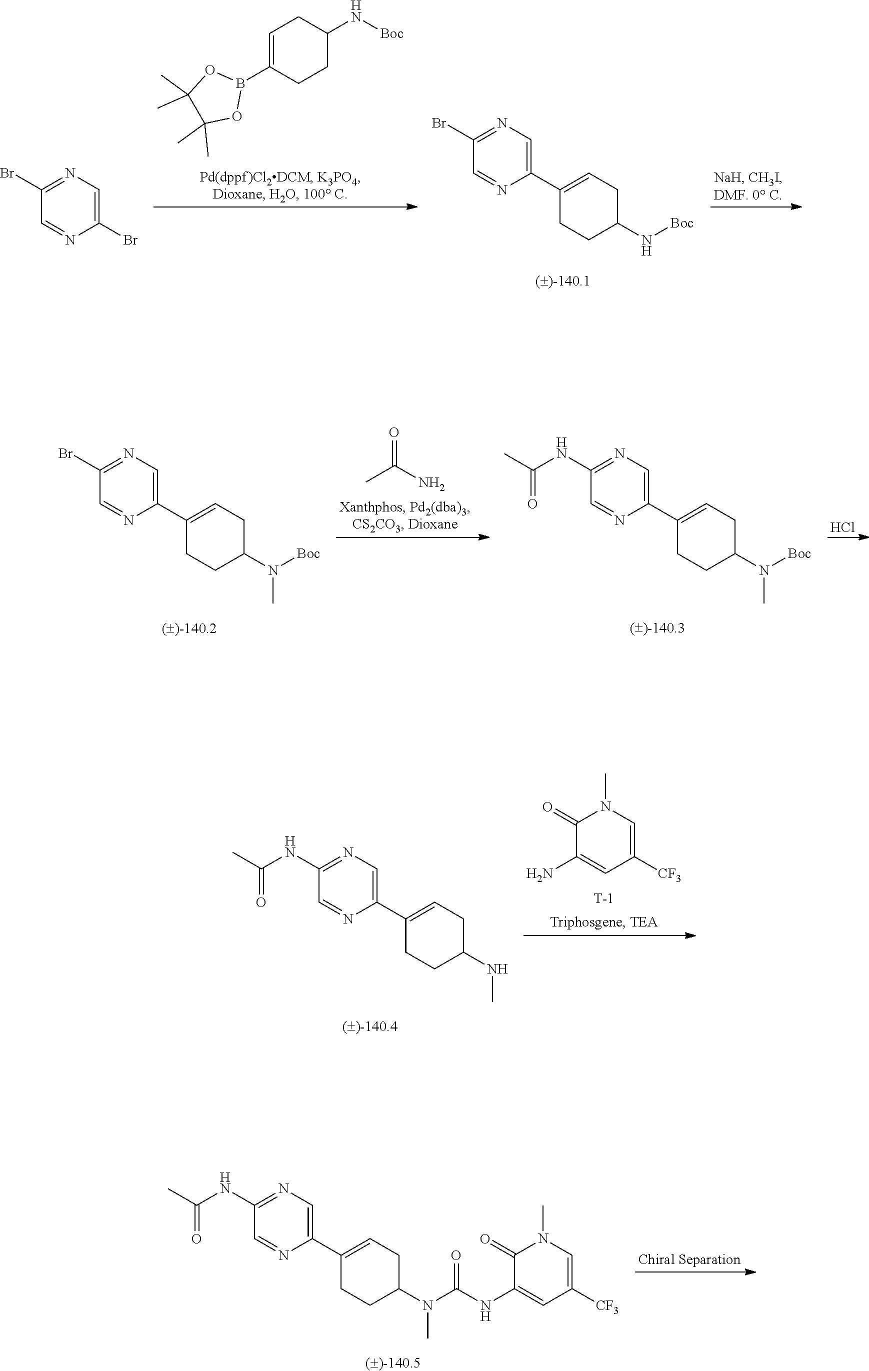

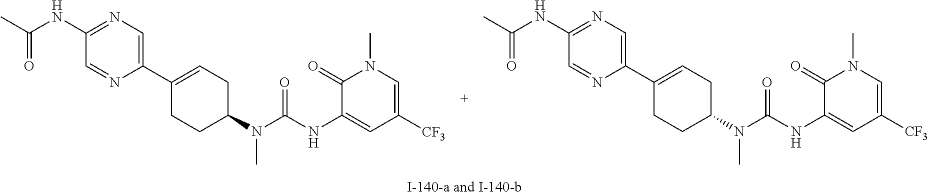 [SMILES: CC(=O)Nc1cnc(C2=CC[C@@H](N(C)C(=O)Nc3cc(C(F)(F)F)cn(C)c3=O)CC2)cn1 ; CC(=O)Nc1cnc(C2=CC[C@H](N(C)C(=O)Nc3cc(C(F)(F)F)cn(C)c3=O)CC2)cn1]

Synthesis of compound (±)-140.1. To a solution of 2,5-dibromopyrazine (1 g, 4.21 mmol, 1.0 equiv) in dioxane (14 mL) and water (6 mL) were added potassium phosphate (2.2 g, 10.54 mmol, 2.5 equiv) and tert-butyl (4-(4,4,5,5-tetramethyl-1,3,2-dioxaborolan-2-yl)cyclohex-3-en-1-yl)carbamate (1.3 g, 4.21 mmol, 1 equiv) followed by the addition of [1,1′-bis(diphenyl phosphino)ferrocene]dichloropalladium(II) complex with DCM (0.34 g, 0.42 mmol, 0.1 equiv). The reaction mixture was stirred at 110° C. for 3 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 32% ethyl acetate in hexane) to afford (±)-140.1 (0.8 g, 54%). MS(ES): m/z 354.25 [M+H]⁺.

Synthesis of compound (±)-140.2. To a solution of compound (±)-140.1 (0.8 g, 2.17 mmol, 1.0 equiv) in DMF (10 mL) was added in portions sodium hydride (0.173 g, 4.34 mmol, 2 equiv) and stirred for 30 min at 0° C. To the mixture was added methyl iodide (0.616 g, 4.34 mmol, 2 equiv). It was stirred at rt for 30 min, transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 29% ethyl acetate in hexane) to afford (f)-140.2 (0.6 g, 72%). MS(ES): m/z 368.27 [M+H]⁺.

Synthesis of compound (±)-140.3. To a solution of (±)-140.2 (0.6 g, 1.63 mmol, 1.0 equiv) and acetamide (0.192 g, 3.26 mmol, 2.0 equiv) in 1,4-dioxane (9 mL) was degassed by bubbling through a stream of argon for 10 min. 4,5-Bis(diphenylphosphoryl)-9,9-dimethylxanthene (0.188 g, 0.326 mmol, 0.2 equiv), cesium carbonate (1.58 g, 4.89 mmol, 3.0 equiv) and tris(dibenzylideneacetone)dipalladium(O) (0.149 g, 0.163 mmol, 0.1 equiv) were added, and degassed for 5 min. The reaction mixture was stirred at 100° C. for 2 h. It was cooled to room temperature, transferred into ice-water, and extracted with ethyl acetate. The combined organic layer was washed with brine solution, dried over sodium sulfate and concentrated under reduced pressure. The residue was purified by flash column chromatography on silica gel (CombiFlash®, 39% ethyl acetate in hexane) to afford (±)-140.3 (0.30 g, 55%). MS(ES): m/z: 346 [M+H]⁺.

Synthesis of compound (±)-140.4. To a solution of (±)-140.3 (0.300 g, 0.867 mmol, 1.0 equiv) in DCM (15 mL) was added hydrochloric acid (4.0 M in dioxane, 7 mL) at 0° C. The reaction mixture was stirred at room temperature for 4 h. It was concentrated under reduced pressure to afford (±)-140.4 (0.16 g, 75%). MS(ES): m/z 246.31 [M+H]⁺.

Synthesis of compound (±)-140.5. Compound (±)-140.5 was prepared from compound (±)-140.4 and T-1 following the procedure described in the synthesis of I-9. The product was purified by flash column chromatography on silica gel (CombiFlash®, 3.3% methanol in DCM). MS(ES): m/z 464.45 [M+H]⁺.

I-140-a and I-140-a: The racemate was separated with chiral SFC (Method D) to afford first eluting fraction (I-140-a) and second eluting fraction (I-140-b).

I-140-a: MS(ES): m/z: 570.53 [M+H]⁺, ¹H-NMR (400 MHz, DMSO-d₆): δ 10.78 (s, 1H), 9.24 (s, 1H), 8.59 (s, 1H), 8.18 (s, 1H), 8.08-8.05 (d, J=12.8 Hz, 1H), 7.94 (s, 1H), 6.73 (s, 1H), 4.35 (s, 1H), 3.60 (s, 3H), 2.93 (s, 3H), 2.83-2.71 (m, 2H), 2.49-2.32 (m, 2H), 2.14 (s, 3H), 1.87 (m, 2H).

I-140-b: MS(ES): m/z: 570.53 [M+H]⁺, ¹H-NMR (400 MHz, DMSO-d₆): δδ 10.77 (s, 1H), 9.23 (s, 1H), 8.58-8.57 (d, J 4, 1H), 8.18-8.17 (d, J 4, 1H), 8.04 (s, 1H), 7.93 (s, 1H), 6.73-6.71 (m, 1H), 5.76 (s, 1H), 4.35-4.34 (m, 1H), 3.59 (s, 3H), 2.92 (s, 3H), 2.83-2.79 (m, 2H), 2.43-2.32 (m, 4H), 2.13 (s, 3H), 1.91-1.85 (m, 2H).

### Example 142: cis-1-methyl-3-(1-methyl-2-oxo-5-(trifluoromethyl)-1,2-dihydropyridin-3-yl)-1-(4-(7-morpholino-5H-pyrrolo[2,3-b]pyrazin-2-yl)cyclohexyl)urea

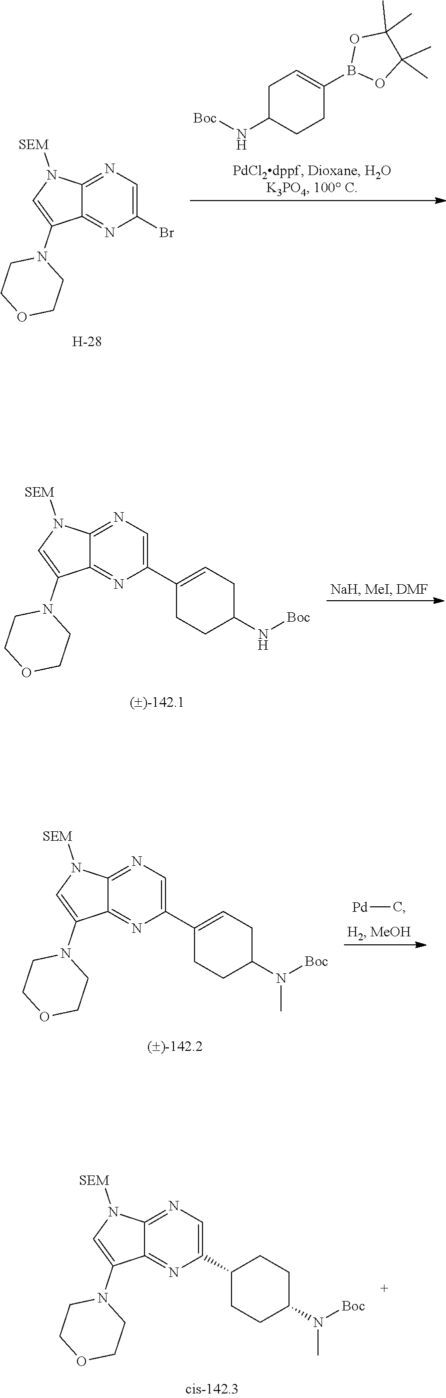

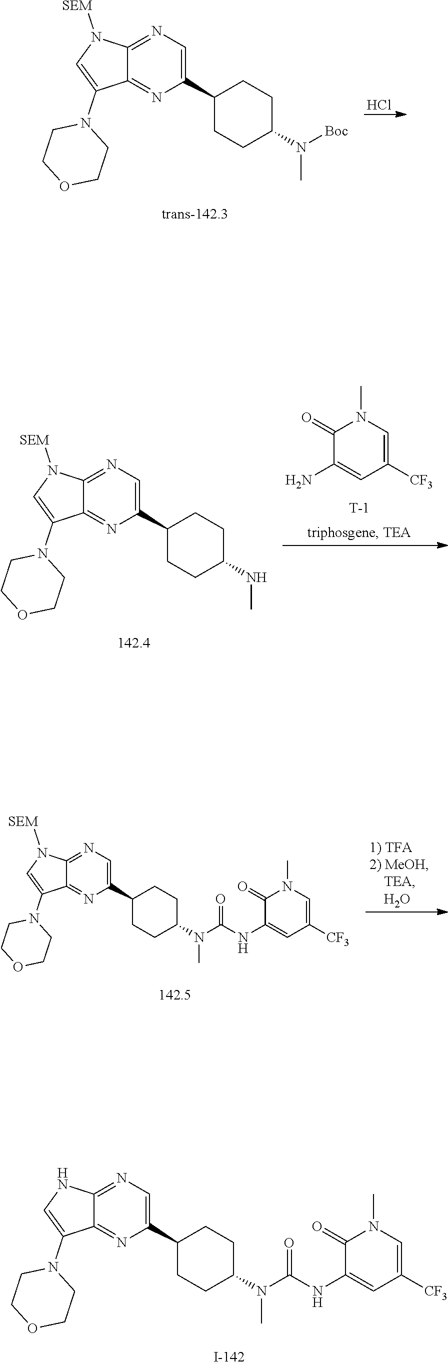

Synthesis of compound (±)-142.1. Compound (±)-142.1 was prepared from compound H-28 and tert-butyl (4-(4,4,5,5-tetramethyl-1,3,2-dioxaborolan-2-yl)cyclohex-3-en-1-yl)carbamate (1.56 g, 7.24 mmol, 1.5 equiv) following the procedure described in the synthesis of (±)-140.1. The product was purified by flash column chromatography on silica gel (CombiFlash®, 32% ethyl acetate in hexane). MS(ES): m/z 529.76 [M+H]⁺.

Synthesis of compound (±)-142.2. To a solution of compound (±)-142.1 (0.7 g, 1.32 mmol, 1.0 equiv) in DMF (15 mL) at 0° C. was added sodium hydride (0.105 g, 2.64 mmol, 2 equiv) in portions. The reaction mixture was stirred for 30 min and methyl iodide (0.374 g, 2.64 mmol, 2 equiv) was added. The reaction mixture was stirred at rt for 30 min, transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 22% ethyl acetate in hexane) to afford (±)-142.2 (0.6 g, 84%). MS(ES): m/z 543.78 [M+H]⁺.

Synthesis of compound cis-142.3 and trans-142.3. A mixture of compound (f)-142.2 (0.6 g, 1.10 mmol, 1.0 equiv), methanol (20 mL) and palladium on carbon (10 wt %, 0.5 g) was stirred under hydrogen for 3 h. The reaction mixture was filtered through a pad of Celite® and rinsed with methanol. The filtrate was concentrated under reduced pressure to afford cis-142.3 (0.19 g, 32%) and trans-142.3 (0.15 g, 25%) MS(ES): m/z 545.86 [M+H]⁺.

Synthesis of compound 142.4. To a solution of trans-142.3 (0.15 g, 0.274 mmol, 1.0 equiv) in DCM (7 mL) was added hydrochloric acid (2.0 M in diethyl ether, 7 mL) at 0° C. The reaction mixture was stirred at room temperature for 4 h. It was concentrated under reduced pressure to afford 142.4 (0.11 g, 90%). MS(ES): m/z 445.68 [M+H]⁺.

Synthesis of compound 142.5. Compound 142.5 was prepared from compound 142.4 and T-1 following the procedure described in the synthesis of I-9. The product was purified by flash column chromatography on silica gel (CombiFlash®, 3.3% methanol in DCM). MS(ES): m/z 663.82 [M+H]⁺.

Synthesis of compound I-142. Compound I-142 was prepared from compound 142.5 following the procedure described in the synthesis of I-11. The product was purified by flash column chromatography on silica gel (CombiFlash®, 3.6% methanol in DCM). MS(ES): m/z: 533.66 [M+H]⁺, ¹H NMR (DMSO-d₆, 400 MHz): δ 11.31 (s, 1H), 8.19-8.18 (d, J 4 Hz, 1H), 8.15 (s, 1H), 8.04 (s, 1H), 7.90 (s, 1H), 7.17-7.16 (d, J 4 Hz, 1H), 3.82-3.80 (m, 4H) 3.59 (s, 3H), 3.22 (s, 3H), 2.91 (m, 2H), 2.89-2.87 (m, 2H), 2.74-2.73 (m, 3H), 1.99-1.97 (d, J 8, 2H), 1.74-1.73 (m, 2H), 1.34-1.29 (m, 2H), 1.67-1.64 (m, 2H).

### Example 143: trans-1-methyl-3-(1-methyl-2-oxo-5-(trifluoromethyl)-1,2-dihydropyridin-3-yl)-1-(4-(7-morpholino-5H-pyrrolo[2,3-b]pyrazin-2-yl)cyclohexyl)urea

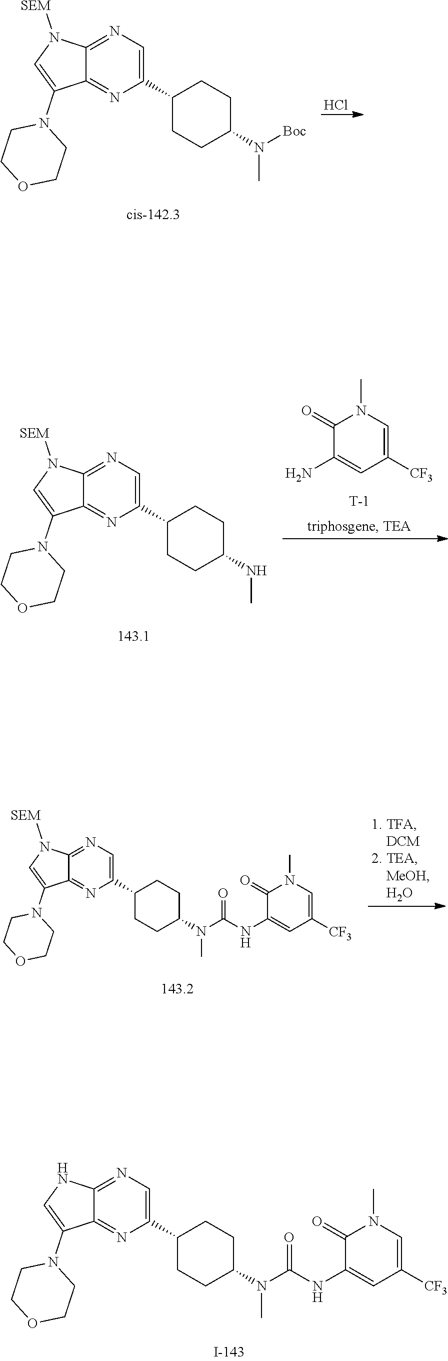

Synthesis of I-143. Compound I-143 was prepared from compound cis-142.3 following the procedure described in the synthesis of I-142. The product was purified by flash column chromatography on silica gel (CombiFlash®, 3.6% methanol in DCM). MS(ES): m/z: 533.66 [M+H]⁺, ¹H NMR (DMSO-d₆, 400 MHz): δ 11.34 (s, 1H), 8.22 (s, 1H), 8.17-8.16 (d, J 4 Hz, 1H), 8.03 (s, 1H), 7.87 (s, 1H), 7.19-7.18 (d, J 4 Hz, 1H), 3.81-3.79 (m, 4H) 3.59 (s, 3H), 3.34 (s, 3H), 2.78 (m, 2H), 2.68 (s, 1H), 2.49-2.22 (m, 4H), 2.01-1.90 (m, 3H), 1.48-1.45 (m, 2H), 1.24 (m, 2H).

### Example 144: trans-1-methyl-3-(1-methyl-2-oxo-5-(trifluoromethyl)-1,2-dihydropyridin-3-yl)-1-(4-(7-(methylamino)-5H-pyrrolo[2,3-b]pyrazin-2-yl)cyclohexyl)urea

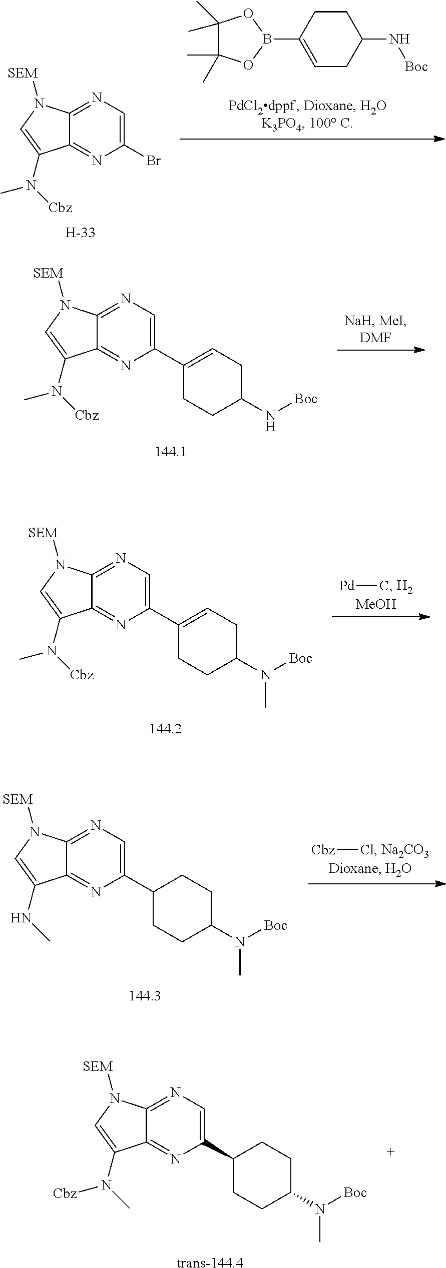

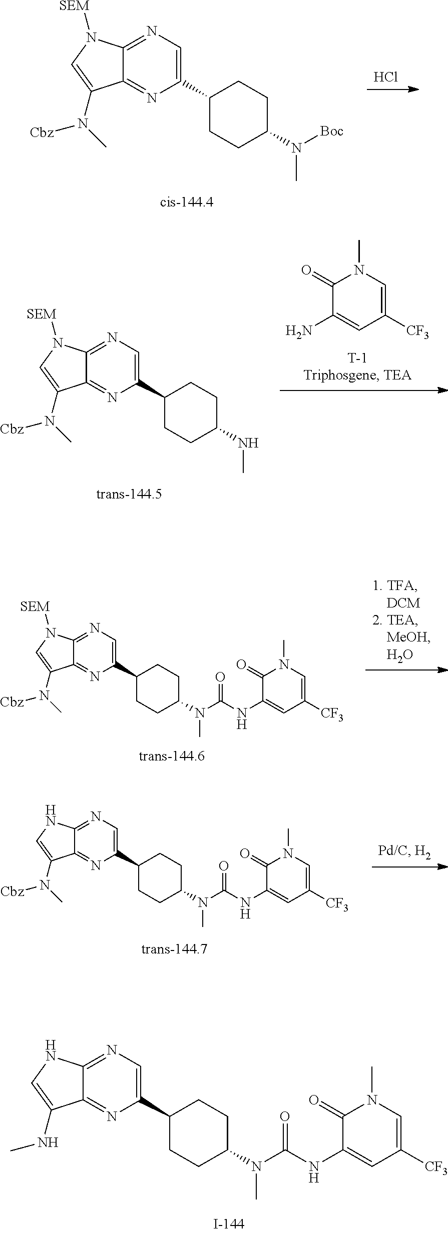

Synthesis of compound 144.1. Compound 144.1 was prepared from compound H-33 and tert-butyl (4-(4,4,5,5-tetramethyl-1,3,2-dioxaborolan-2-yl)cyclohex-3-en-1-yl)carbamate (1.56 g, 7.24 mmol, 1.5 equiv) following the procedure described in the synthesis of (±)-140.1. The product was purified by flash column chromatography on silica gel (CombiFlash®, 32% ethyl acetate in hexane). MS(ES): m/z 607.83 [M+H]⁺.

Synthesis of compound 144.2. To a solution of 144.1 (1.5 g, 2.46 mmol, 1.0 equiv) in DMF (15 mL) was added sodium hydride (0.19 g, 4.94 mmol, 2 equiv) in portions and stirred for 30 min at 0° C. To the mixture was added dropwise methyl iodide (0.701 g, 4.94 mmol, 2 equiv) and stirred at rt for 30 min. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 22% ethyl acetate in hexane) to afford 144.2 (1.1 g, 72%). MS(ES): m/z 621.85 [M+H]⁺.

Synthesis of compound 144.3. A mixture of 144.2 (1.1 g, 1.17 mmol, 1.0 equiv), methanol (20 mL) and palladium on carbon (10 wt %, 0.7 g) was stirred under hydrogen for 3 h. It was filtered through a pad of Celite® and rinsed with methanol. The filtrate was concentrated under reduced pressure to afford 144.3 (0.701 g, 81%). MS(ES): m/z 489.7 [M+H]⁺.

Synthesis of compound trans-144.4 and cis-144.4. To a solution of 144.3 (0.7 g, 1.43 mmol, 1.0 equiv) in dioxane (20 mL) was added saturated sodium carbonate solution in water (7 mL) at 0° C. The reaction mixture was stirred for 10 min at 0° C. and was added benzyl chloroformate (50% solution in toluene, 0.97 mL, 2.8 mmol, 2 equiv). The reaction mixture was stirred for 30 min at 0° C. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.9% methanol in DCM) to afford trans-144.4 (0.29 g, 35%) and cis-144.4 (0.31 g, 36%). MS(ES): m/z 623.87 [M+H]⁺.

Synthesis of compound trans-144.5. To a solution of trans-144.4 (0.29 g, 0.465 mmol, 1.0 equiv) in DCM (5 mL) was added hydrochloric acid (4.0 M in dioxane, 1 mL) at 0° C. The reaction mixture was stirred at room temperature for 4 h. It was concentrated under reduced pressure to afford trans-144.5 (0.16 g, 73%). MS(ES): m/z 523.75 [M+H]⁺.

Synthesis of compound trans-144.6. Compound trans-144.6 was prepared from compound trans-144.5 and T-1 following the procedure described in the synthesis of I-9. The product was purified by flash column chromatography on silica gel (CombiFlash®, 2.3% methanol in DCM). MS(ES): m/z 741.89 [M+H]⁺.

Synthesis of compound trans-144.7. Compound trans-144.7 was prepared from compound trans-144.6 following the procedure described in the synthesis of I-11. The product was purified by flash column chromatography on silica gel (CombiFlash®, 4.1% methanol in DCM). MS(ES): m/z 611.63 [M+H]⁺.

Synthesis of I-144. A mixture of compound trans-144.7 (0.068 g, 0.11 mmol, 1.0 equiv), methanol (10 mL) and palladium on carbon (10 wt %, 0.050 g) was stirred under hydrogen for 3 h. The reaction mixture was filtered through a pad of Celite® and rinsed with methanol. The filtrate was concentrated under reduced pressure. The residue was purified by flash column chromatography on silica gel (CombiFlash®, 3% methanol in DCM) to afford I-144 (0.011 g, 21%) MS(ES): m/z: 477.49 [M+H]⁺, ¹H NMR (DMSO-d₆, 400 MHz): δ 10.93 (s, 1H), 8.19-8.18 (d, J 4.4 Hz, 1H), 8.11 (s, 1H), 8.03 (s, 1H), 6.93-6.92 (d, J 4 Hz, 1H), 4.64-4.63 (m, 1H) 4.11-4.10 (m, 1H), 3.60 (s, 3H), 2.92 (s, 3H), 2.81 (m, 1H), 2.74-2.73 (m, 3H), 2.00-1.97 (m, 2H), 1.82-1.74 (m, 2H).

### Example 145: 1-methyl-3-(1-methyl-2-oxo-5-(trifluoromethyl)-1,2-dihydropyridin-3-yl)-1-((1s,4s)-4-(7-(methylamino)-5H-pyrrolo[2,3-b]pyrazin-2-yl)cyclohexyl)urea

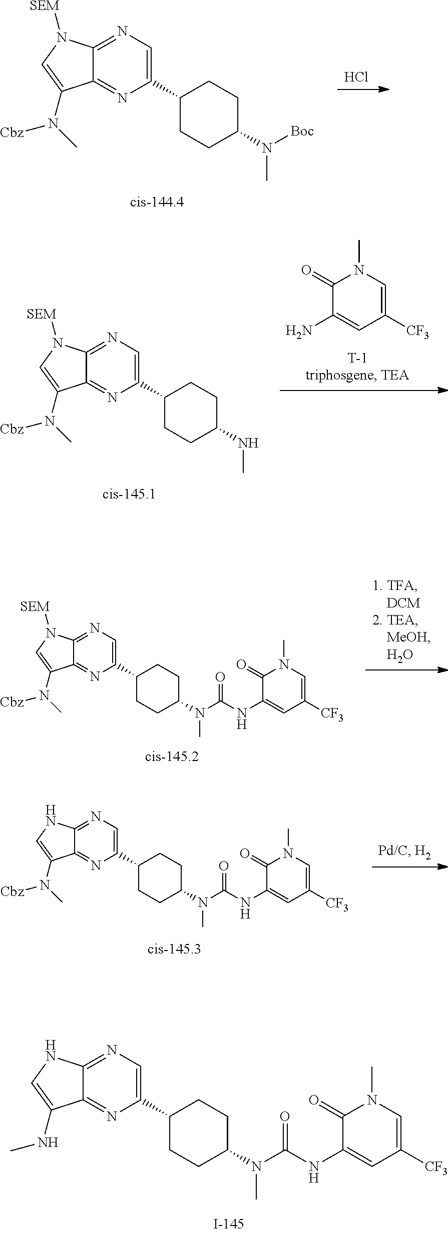

Synthesis of compound cis-145.1. To a solution of cis-144.4 (0.31 g, 0.49 mmol, 1.0 equiv) in DCM (5 mL) was added hydrochloric acid (4.0 M in dioxane, 1.5 mL) at 0° C. The reaction mixture was stirred at room temperature for 4 h. It was concentrated under reduced pressure to afford cis-145.1 (0.16 g, 73%). MS(ES): m/z 523.75 [M+H]⁺.

Synthesis of compound cis-145.2. Compound cis-145.2 was prepared from compound cis-145.1 and T-1 following the procedure described in the synthesis of I-9. The product was purified by flash column chromatography on silica gel (CombiFlash®, 2.3% methanol in DCM). MS(ES): m/z 741.89 [M+H]⁺.

Synthesis of compound cis-145.3. Compound cis-145.3 was prepared from compound cis-145.2 following the procedure described in the synthesis of I-11. The product was purified by flash column chromatography on silica gel (CombiFlash®, 4.1% methanol in DCM). MS(ES): m/z 611.63 [M+H]⁺.

Synthesis of compound 1-145. A mixture of compound cis-145.3 (0.069 g, 0.11 mmol, 1.0 equiv), methanol (10 mL) and palladium on carbon (10 wt %, 0.050 g) was stirred under hydrogen for 3 h. The reaction mixture was filtered through a pad of Celite® and rinsed with methanol. The filtrate was concentrated under reduced pressure. The residue was purified by flash column chromatography on silica gel (CombiFlash®, 3% methanol in DCM) to afford I-145 (0.011 g, 21%) MS(ES): m/z: 477.49 [M+H]⁺, ¹H NMR (DMSO-d₆, 400 MHz): δ 10.95 (s, 1H), 8.20-8.19 (d, J 4.4 Hz, 1H), 8.12 (s, 1H), 8.04 (s, 1H), 6.93-6.92 (d, J 4.0 Hz, 1H), 4.64-4.63 (m, 1H) 4.12-4.11 (m, 1H), 3.60 (s, 3H), 2.93 (s, 3H), 2.82 (m, 1H), 2.73-2.74 (m, 3H), 2.01-1.98 (m, 2H), 1.83-1.75 (m, 2H).

### Example 146: (R)-1-(3,3-difluoro-1-(1H-pyrazolo[3,4-b]pyridin-5-yl)piperidin-4-yl)-1-methyl-3-(4-(trifluoromethyl)pyridin-2-yl)urea and (S)-1-(3,3-difluoro-1-(1H-pyrazolo[3,4-b]pyridin-5-yl)piperidin-4-yl)-1-methyl-3-(4-(trifluoromethyl)pyridin-2-yl)urea

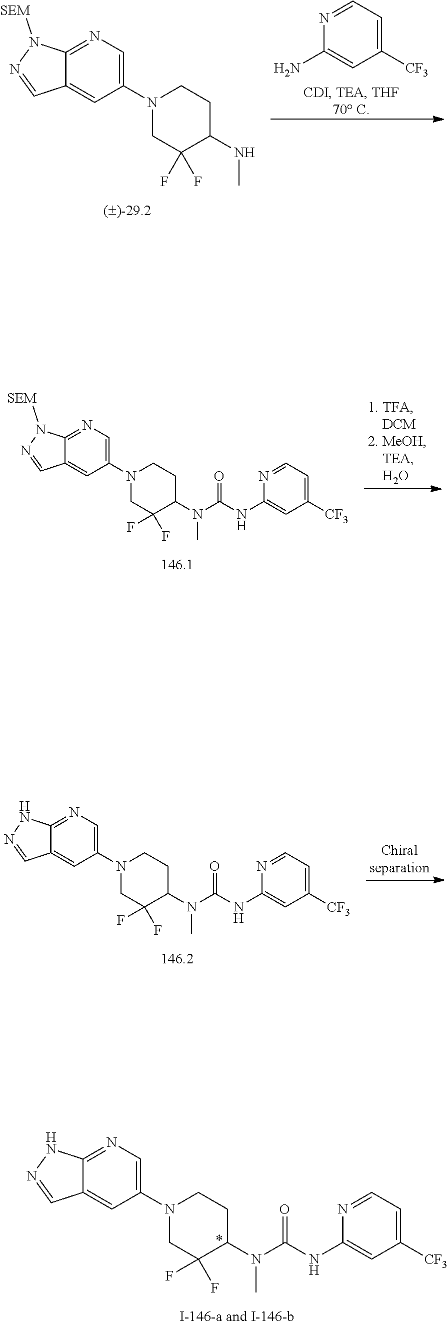

Synthesis of compound 146.1. To a solution of compound (±)-29.2 (0.350 g, 0.88 mmol, 1.0 equiv), 4-(trifluoromethyl)pyridin-2-amine (0.185 g, 1.14 mmol, 1.3 equiv) and triethylamine (0.88 g, 8.8 mmol, 10 equiv) in THE (15 mL) was added 1,1′-carbonyldiimidazole (0.714 g, 4.4 mmol, 5 equiv). The reaction mixture was stirred at 70° C. for 2 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford 146.1 (0.12 g, 23%). MS (ES): m/z 586.6 [M+H]⁺.

Synthesis of compound 146.2. Compound 146.2 was prepared from compound 146.1 following the procedure described in the synthesis of I-11. The product was purified by flash column chromatography on silica gel (CombiFlash®, 3.7% methanol in DCM). MS(ES): m/z 456.3 [M+H]⁺.

I-146-a and 1-146-a. The racemate was separated with chiral SFC (Method D) to afford first eluting fraction (1-146-a) and second eluting fraction (1-146-b).

I-146-a: MS(ES): m/z 456.3 [M+H]⁺, ¹H NMR (DMSO-d₆, 400 MHz): δ 13.47 (s, 1H), 9.57 (s, 1H), 8.55-8.54 (d, J 5.2 Hz, 1H), 8.50-8.49 (d, J 2.4 Hz, 1H), 8.19 (s, 1H), 8.00 (s, 1H), 7.74-7.73 (d, J 2.8 Hz, 1H), 7.38-7.36 (d, J 5.2 Hz, 1H), 4.94-4.84 (m, 1H), 3.93-3.87 (t, J 12 Hz, 1H), 3.79-3.75 (m, 1H), 3.30-3.19 (m, 1H), 3.05-3.02 (m, 4H), 2.37-2.27 (m, 1H), 1.90-1.87 (m, 1H).

I-146-b: MS(ES): m/z 456.3 [M+H]⁺, ¹H NMR (DMSO-d₆, 400 MHz): δ 13.47 (s, 1H), 9.57 (s, 1H), 8.55-8.54 (d, J 5.2 Hz, 1H), 8.50-8.49 (d, J 2.4 Hz, 1H), 8.19 (s, 1H), 8.00 (s, 1H), 7.74-7.73 (d, J 2.4 Hz, 1H), 7.38-7.36 (d, J 4.8 Hz, 1H), 4.94-4.84 (m, 1H), 3.93-3.87 (t, J 12 Hz, 1H), 3.80-3.77 (m, 1H), 3.30-3.19 (m, 1H), 3.05-3.02 (m, 4H), 2.40-2.27 (m, 1H), 1.91-1.87 (m, 1H).

### Example 147: (R)-1-(3,3-difluoro-1-(1H-pyrazolo[3,4-b]pyridin-5-yl)piperidin-4-yl)-1-methyl-3-(5-(trifluoromethyl)pyridin-3-yl)urea and (S)-1-(3,3-difluoro-1-(1H-pyrazolo[3,4-b]pyridin-5-yl)piperidin-4-yl)-1-methyl-3-(5-(trifluoromethyl)pyridin-3-yl)urea

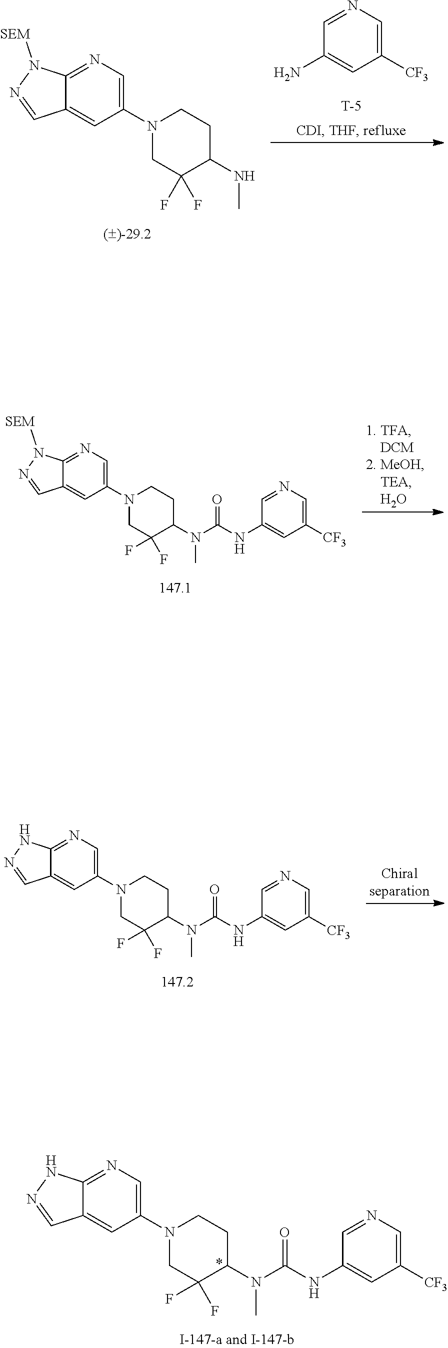

Synthesis of compound 147.1. To a solution of compound (±)-29.2 (0.142 g, 0.356 mmol, 1.0 equiv) in THF (10 mL) was added CDI (0.288 g, 1.78 mmol, 5.0 equiv) and stirred at reflux temperature for 5 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 2% methanol in DCM) to afford 147.1 (0.12 g, 23%). MS (ES): m/z 586.6 [M+H]⁺.

Synthesis of compound 147.2. Compound 147.2 was prepared from compound 147.1 following the procedure described in the synthesis of I-11. The product was purified by flash column chromatography on silica gel (CombiFlash®, 3.7% methanol in DCM) to afford 147.2 (0.050 g, 54%). MS(ES): m/z 456.3 [M+H]⁺.

I-147-a I-147-b. The racemate was separated with chiral SFC (Method F) afford first eluting fraction (I-147-a) and second eluting fraction (I-147-b).

I-147-a: MS(ES): m/z 456.3 [M+H]⁺, ¹H NMR (DMSO-d₆, 400 MHz): δ 13.446 (s, 1H), 9.021-8.998 (d, J 9.2 Hz, 2H), 8.56 (s, 1H), 8.49 (s, 1H), 8.35 (s, 1H), 7.99 (s, 1H), 7.73 (s, 1H), 4.91-4.81 (m, 1H), 3.93-3.87 (m, 1H), 3.79-3.76 (d, J 11.2 Hz, 1H), 3.28-3.14 (m, 1H), 3.021-3.06 (m, 4H), 2.35-2.26 (m, 1H), 1.88-1.83 (m, 1H).

I-147-b: MS(ES): m/z 456.3 [M+H]⁺, ¹H NMR (DMSO-d₆, 400 MHz): δ 13.45 (s, 1H), 9.03 (s, 1H), 9.00 (s, 1H), 8.56 (s, 1H), 8.49-8.48 (d, J 2.8 Hz, 1H), 8.36 (s, 1H), 7.99 (s, 1H), 7.73-7.72 (d, J 2.4 Hz, 1H), 4.91-4.81 (m, 1H), 3.93-3.87 (m, 1H), 3.79-3.77 (d, J 11.2 Hz, 1H), 3.28-3.25 (m, 1H), 3.01-3.06 (m, 4H), 2.32-2.26 (m, 1H), 1.88-1.83 (m, 1H).

### Example 148: ((4aS,8aS)-6-(5-(cyclopropanecarboxamido)pyrazin-2-yl)-N-(1-methyl-2-oxo-5-(trifluoromethyl)-1,2-dihydropyridin-3-yl)octahydro-1H-pyrido[3,4-b][1,4]oxazine-1-carboxamide and (4aR,8aR)-6-(5-(cyclopropanecarboxamido)pyrazin-2-yl)-N-(1-methyl-2-oxo-5-(trifluoromethyl)-1,2-dihydropyridin-3-yl)octahydro-1H-pyrido[3,4-b][1,4]oxazine-1-carboxamide

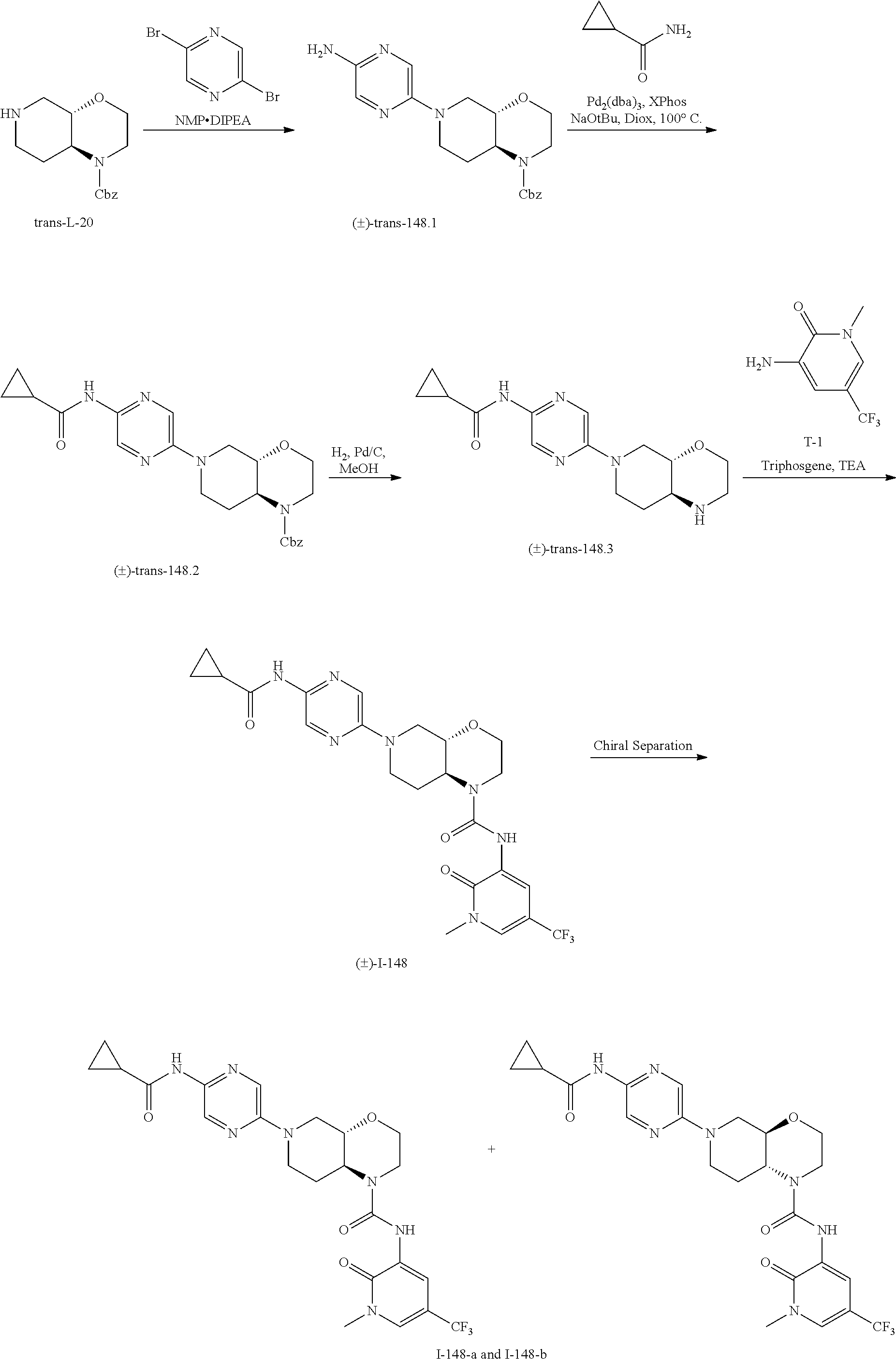

Synthesis of compound (±)-trans-148.1. To a solution of trans-L-20 (0.195 g, 0.705 mmol, 1.0 equiv) in dimethyl sulfoxide (3 mL) at 0° C. was added N,N-diisopropylethylamine (0.273 g, 2.11 mmol, 3.0 equiv) and stirred for 15 min. To the mixture was added 2,5-dibromopyrazine (0.168 g, 0.705 mmol, 1.0 equiv) and stirred at room temperature for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (20% ethyl acetate in hexane) to afford (±)-trans-148.1 (0.150 g, 49%). MS(ES): m/z 433.31 [M+H]⁺.

Synthesis of compound (±)-trans-148.2. Compound (±)-trans-148.2 was prepared from compound (±)-trans-148.1 and cyclopropanecarboxamide following the procedure described in the synthesis of 11.1. The product was purified by flash column chromatography on silica gel (CombiFlash®, 60% ethyl acetate in hexane). MS(ES): m/z 437.50 [M+H]⁺.

Synthesis of compound (±)-trans-148.3. A mixture of (±)-trans-148.2 (0.120 g, 0.274 mmol, 1.0 equiv), methanol (10 mL) and palladium on carbon (10 wt %, 0.075 g) was stirred under hydrogen (1 atm) for 5 h. The reaction mixture was filtered through a pad of Celite® and rinsed with methanol. The filtrate was concentrated under reduced pressure to afford (±)-trans-148.3 (0.080 g, 96%). MS(ES): m/z 303.37 [M+H]⁺.

Synthesis of compound (±)-I-148. Compound (±)-I-148 was prepared from compound (±)-trans-148.3 and T-1 following the procedure described in the synthesis of I-9. The product was purified by flash column chromatography on silica gel (2.2% methanol in DCM). MS(ES): m/z 521.50 [M+H]⁺.

I-148-a and I-148-b. The racemate was separated by chiral HPLC (Method G) to afford first eluting fraction (I-148-a) and second eluting fraction (I-148-b).

I-148-a: MS(ES): m/z: 521.50 [M+H]⁺, ¹H NMR (DMSO-d₆, 400 MHz): δ 10.63 (s, 1H), 8.78 (s, 1H), 8.28 (s, 1H), 8.15 (s, 1H), 8.08 (s, 1H), 8.06 (s, 1H), 4.40-4.37 (m, 1H), 4.29-4.26 (m, 1H), 3.93-3.91 (m, 1H), 3.79-3.76 (m, 2H), 3.58 (s, 3H), 3.54 (m, 1H), 2.90 (m, 1H), 2.74-2.71 (m, 2H), 1.93-1.87 (m, 1H), 1.40-1.35 (m, 2H), 1.34-1.30 (m, 1H), 0.80-0.78 (m, 4H).

I-148-b: MS(ES): m/z: 521.50 [M+H]⁺, ¹H NMR (DMSO-d₆, 400 MHz): δ 10.62 (s, 1H), 8.77 (s, 1H), 8.27 (s, 1H), 8.15 (s, 1H), 8.08 (s, 1H), 8.05 (s, 1H), 4.39-4.36 (m, 1H), 4.27-4.24 (m, 1H), 3.93-3.90 (m, 1H), 3.74-3.72 (m, 2H), 3.57 (s, 3H), 3.53-5.52 (m, 1H), 3.40-3.37 (m, 1H), 2.89 (m, 1H), 2.76-2.73 (m, 1H), 2.60-2.58 (m, 1H), 1.94-1.90 (m, 1H), 1.59 (m, 1H), 1.39 (m, 1H), 0.79-0.76 (m, 4H).

### Example 165: (S)-1-methyl-3-(1-methyl-2-oxo-5-(trifluoromethyl)-1,2-dihydropyridin-3-yl)-1-(4-(7-morpholino-5H-pyrrolo[2,3-b]pyrazin-2-yl)cyclohex-3-en-1-yl)urea and (R)-1-methyl-3-(1-methyl-2-oxo-5-(trifluoromethyl)-1,2-dihydropyridin-3-yl)-1-(4-(7-morpholino-5H-pyrrolo[2,3-b]pyrazin-2-yl)cyclohex-3-en-1-yl)urea

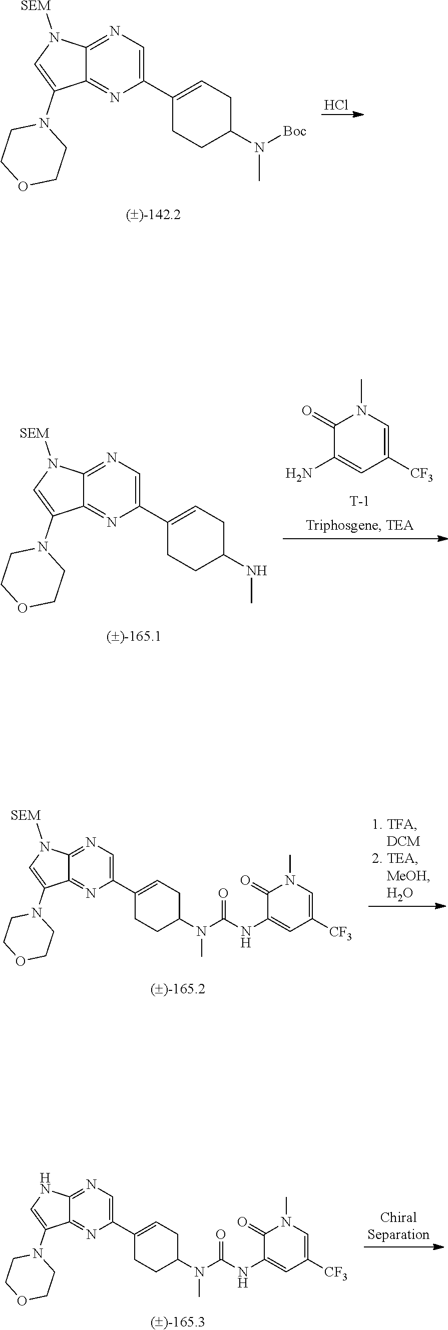

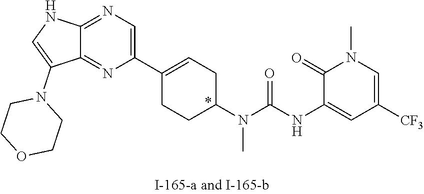 [SMILES: CN(C(=O)Nc1cc(C(F)(F)F)cn(C)c1=O)C1CC=C(c2cnc3[nH]cc(N4CCOCC4)c3n2)CC1]

Synthesis of compound (±)-165.1. To a solution of (±)-142.2 (0.212 g, 0.389 mmol, 1.0 equiv) in DCM (5 mL) was added 4 M hydrochloric acid in 1,4-dioxane (2 mL) dropwise at 0° C. The reaction mixture was stirred at room temperature for 30 min. It was transferred into a saturated aqueous solution of sodium bicarbonate and extracted with DCM. The combined organic layers were washed with brine, dried over anhydrous sodium sulfate, filtered, and concentrated under reduced pressure to afford (±)-165.1 (0.112 g, 65%). MS(ES): m/z 444.67 [M+H]⁺.

Synthesis of compound (±)-165.2. Compound (±)-165.2 was prepared from compound (±)-trans-148.3 and T-1 following the procedure described in the synthesis of I-9. The product was purified by flash column chromatography on silica gel (CombiFlash®, 1.8% methanol in DCM). MS(ES): m/z 662.81 [M+H]⁺.

Synthesis of compound (±)-165.3. Compound (±)-165.3 was prepared from compound (±)-165.2 following the procedure described in the synthesis of I-11. The product was purified by flash column chromatography on silica gel (CombiFlash®, 3% methanol in DCM). MS(ES): m/z 532.64 [M+H]⁺.

I-165-a and I-165-b. The racemate was separated with chiral SFC (Method H) to afford first eluting fraction (I-165-a) and second eluting fraction (I-165-b).

I-165-a: MS(ES): m/z: 532.3 [M+H]⁺, ¹H NMR (DMSO-d₆, 400 MHz): δ 11.40 (s, 1H), 8.46 (s, 1H), 8.18 (s, 1H), 8.04 (s, 1H), 7.94 (s, 1H), 7.18 (s, 1H), 6.61 (bs, 1H), 4.36-4.34 (m, 1H), 3.80-3.79 (d, J 4.0 Hz, 4H), 3.59 (s, 3H), 3.20 (s, 4H), 2.93 (s, 3H), 1.95-1.91 (m, 2H), 1.89-1.87 (m, 4H), 1.34-1.24 (m, 2H).

I-165-b: MS(ES): m/z: 532.3 [M+H]⁺, ¹H NMR (DMSO-d₆, 400 MHz): δ 11.41 (s, 1H), 8.46 (s, 1H), 8.19 (s, 1H), 8.05 (s, 1H), 7.95 (s, 1H), 7.18 (s, 1H), 6.62 (bs, 1H), 4.36-4.34 (m, 1H), 3.80-3.79 (d, J 4.0 Hz, 4H), 3.59 (s, 3H), 3.20 (s, 4H), 2.94 (s, 3H), 1.96-1.92 (m, 2H), 1.89-1.88 (m, 4H), 1.34-1.23 (m, 2H).

### Example 166: (S)-1-methyl-3-(1-methyl-2-oxo-5-(trifluoromethyl)-1,2-dihydropyridin-3-yl)-1-(3-(3-((tetrahydrofuran-3-yl)oxy)-1H-pyrazolo[3,4-b]pyridin-5-yl)bicyclo[1.1.1]pentan-1-yl)urea and (R)-1-methyl-3-(1-methyl-2-oxo-5-(trifluoromethyl)-1,2-dihydropyridin-3-yl)-1-(3-(3-((tetrahydrofuran-3-yl)oxy)-1H-pyrazolo[3,4-b]pyridin-5-yl)bicyclo[1.1.1]pentan-1-yl)urea

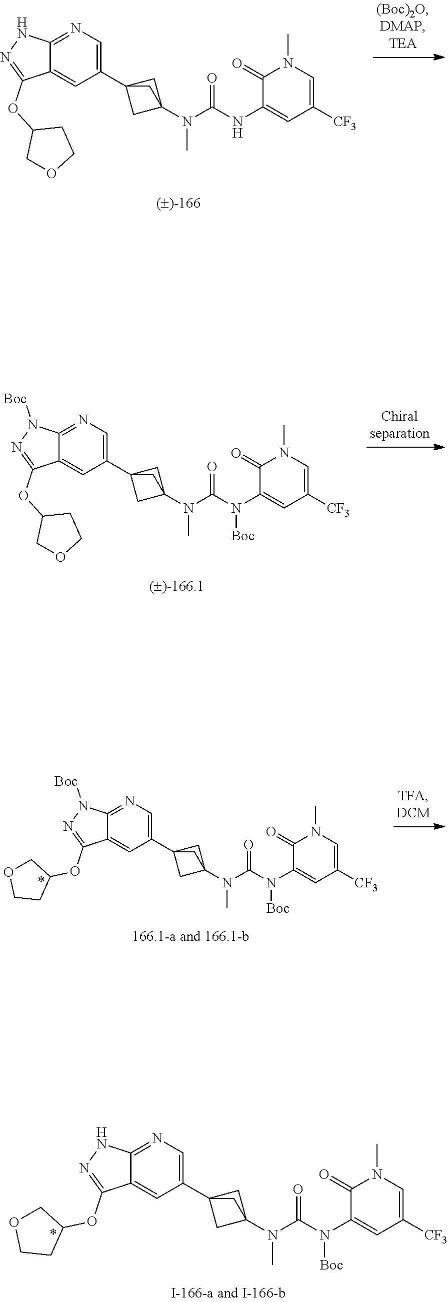 [SMILES: CN(C(=O)Nc1cc(C(F)(F)F)cn(C)c1=O)C12CC(c3cnc4[nH]nc(OC5CCOC5)c4c3)(C1)C2 ; CN(C(=O)N(C(=O)OC(C)(C)C)c1cc(C(F)(F)F)cn(C)c1=O)C12CC(c3cnc4c(c3)c(OC3CCOC3)nn4C(=O)OC(C)(C)C)(C1)C2 ; CN(C(=O)N(C(=O)OC(C)(C)C)c1cc(C(F)(F)F)cn(C)c1=O)C12CC(c3cnc4c(c3)c(OC3CCOC3)nn4C(=O)OC(C)(C)C)(C1)C2 ; CN(C(=O)N(C(=O)OC(C)(C)C)c1cc(C(F)(F)F)cn(C)c1=O)C12CC(c3cnc4[nH]nc(OC5CCOC5)c4c3)(C1)C2]

Synthesis of compound (±)-I-166. To a solution of (±)-166 (0.045 g, 0.0868 mmol, 1.0 equiv), triethylamine (0.025 mL, 0.1737 mmol, 2.0 equiv) and dimethyl aminopyridine (0.001 g, 0.00868 mmol, 0.1 equiv) in THE (2 mL) at 0° C. was added di-tert-butyl dicarbonate (0.02 g, 0.095 mmol, 1.1 equiv) and stirred for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.2% methanol in DCM) to afford (±)-166.1 (0.042 g, 67%). MS(ES): m/z: 719.2 [M+H]⁺.

Compound 166.1-a and 166.1-b: The racemate was separated by chiral SFC (Method B) to afford first eluting fraction (166.1-a), MS(ES): m/z: 719.2 [M+H]⁺; and second eluting fraction (166.1-b), MS(ES): m/z 719.1 [M+H]⁺.

Synthesis of compound I-166-a. To a solution of 166.1-a (0.032 g, 0.044 mmol, 1.0 equiv) in DCM (1 mL) was added trifluoroacetic acid (0.5 mL) at 0° C. and stirred for 1.5 h. It was transferred into ice-saturated sodium bicarbonate solution and extracted with DCM. The combined organic layers were washed with brine, dried over anhydrous sodium sulfate, filtered, and concentrated under reduced pressure. The residue was purified by flash column chromatography on silica gel (CombiFlash®, 1.5% methanol in DCM) to afford I-166-a (0.012 g, 52%) MS(ES): m/z: 519.66 [M+H]*, ¹H-NMR (400 MHz, DMSO-d₆): δ 12.33 (s, 1H), 8.439-8.432 (d, J=2.8 Hz, 1H), 8.16-8.15 (d, J=2.4 Hz, 1H), 8.04 (s, 1H), 7.95 (s, 1H), 7.49-7.48 (d, J=2.4 Hz, 1H), 5.40-5.36 (m, 1H), 3.93-3.87 (m, 2H) 3.80-3.76 (m, 1H), 3.67-3.64 (m, 1H), 2.87 (s, 3H), 2.79-2.74 (m, 2H), 2.34 (s, 3H), 2.27-2.24 (m, 1H), 2.17-2.13 (m, 1H), 1.93-1.90 (m, 2H), 1.68.1.66 (m, 2H).

Synthesis of compound 1-166-b. Compound 1-166-b was prepared from 166.1-b following the procedure in the synthesis of I-166-a. The product was purified by flash column chromatography on silica gel (CombiFlash®, 1.5% methanol in DCM). MS(ES): m/z: 519.67 [M+H]⁺, ¹H-NMR (400 MHz, DMSO-d₆): δ 12.33 (s, 1H), 8.41-8.40 (d, J=2.4 Hz, 1H), 8.19-8.18 (d, J=2.4 Hz, 1H), 7.99 (s, 1H), 7.95 (s, 1H), 7.46-7.45 (d, J=2.4 Hz, 1H), 5.40-5.36 (m, 1H), 3.93-3.87 (m, 2H) 3.80-3.76 (m, 1H), 3.67-3.64 (m, 1H), 2.87 (s, 3H), 2.79-2.74 (m, 2H), 2.34 (s, 3H), 2.27-2.24 (m, 1H), 2.17-2.13 (m, 1H), 1.93-1.90 (m, 2H), 1.68.1.66 (m, 2H).

### Example 167: 1-((3S,4S)-3-(difluoromethoxy)-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-((3R,4R)-3-(difluoromethoxy)-1-(1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

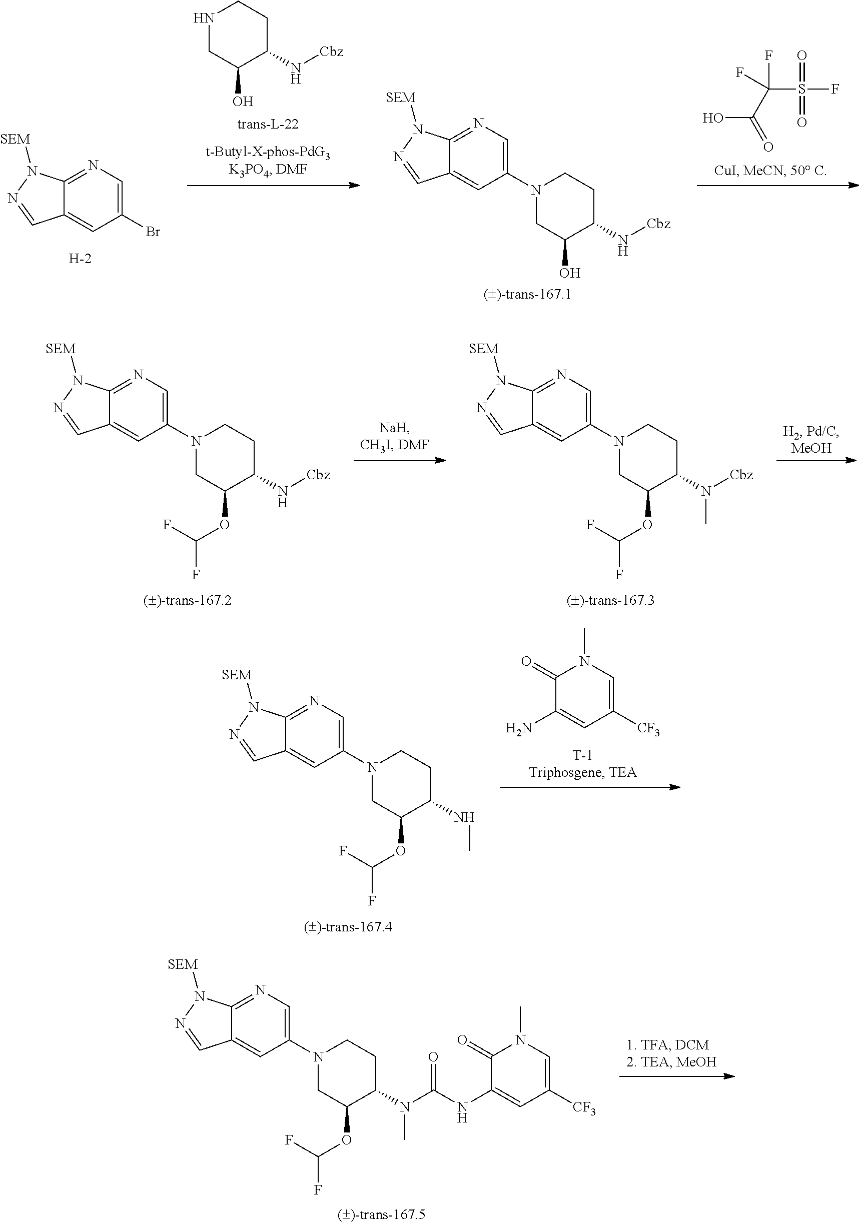

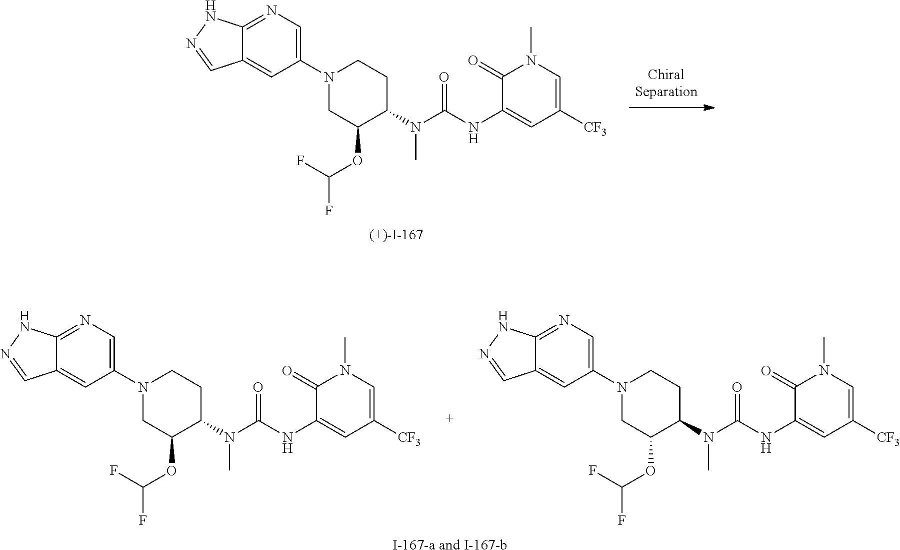

Synthesis of compound (±)-trans-167.1. A mixture of H-2 (0.5 g, 1.524 mmol, 1.0 equiv), trans-L-22 (0.55 g, 3.048 mmol, 2.0 equiv), tert-butyl X-phos Pd G3 (0.35 g, 0.609 mmol, 0.4 equiv) and potassium phosphate (1.5 g, 7.621 mmol, 5.0 equiv) in DMF (15 mL) was degassed by bubbling through a stream of argon for 10 min. The reaction mixture was stirred at rt for 16 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.6% methanol in DCM) to afford (±)-trans-167.1 (0.6 g, 18%). MS(ES): m/z 498.67 [M+H]⁺.

Synthesis of compound (±)-trans-167.2. A mixture of (±)-trans-167.1 (0.60 g, 1.20 mmol, 1.0 equiv), 2,2-difluoro-2-(fluorosulfonyl)acetic acid (0.429 g, 2.41 mmol, 2.0 equiv) and copper(I) iodide (0.0456 g, 0.24 mmol, 0.2 equiv) in acetonitrile (20 mL) was stirred at 50° C. for 1 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 50% ethyl acetate in hexane) to afford (±)-trans-167.2 (0.18 g, 29%). MS(ES): m/z 548.24 [M+H]⁺.

Synthesis of compound (±)-trans-167.3. To a solution of (±)-trans-167.2 (0.18 g, 1.09 mmol, 1.0 equiv) in DMF (10 mL) at 0° C. was added sodium hydride (0.087 g, 2.1 mmol, 2.0 equiv) and stirred for 15 min. To the mixture was added methyl iodide and stirred at rt for 15 min. It was transferred into water and extracted with ethyl acetate. The combined organic layers were washed with brine, dried over anhydrous sodium sulfate, filtered, and concentrated under reduced pressure to afford (±)-trans-167.3 (0.205 g, 75%) MS(ES): m/z 562.26 [M+H]⁺.

Synthesis of compound (±)-trans-167.4. A mixture of (±)-trans-167.3 (0.205 g), methanol (10 mL) and 10% palladium on carbon (0.02 g) was stirred under hydrogen (1 atm) for 2 h. It was filtered through a pad of Celite® and wash with 10% methanol in DCM solution. The filtrate was concentrated under reduced pressure to afford (±)-trans-167.4. (0.155 g, 75%) MS(ES): m/z 428.22 [M+H]⁺.

Synthesis of compound (±)-trans-167.5. Compound (±)-trans-167.5 was prepared from compound (±)-trans-167.4 and T-1 following the procedure described in the synthesis of I-9. The product was purified by flash column chromatography on silica gel (CombiFlash®, 1.8% methanol in DCM). MS(ES): m/z 646.25 [M+H]⁺.

Synthesis of compound (±)-I-167. Compound (±)-I-167 was prepared from compound (±)-trans-167.5 following the procedure described in the synthesis of I-11. The product was purified by flash column chromatography on silica gel (CombiFlash®, 3.2% methanol in DCM). MS(ES): m/z: 516.17 [M+H]⁺.

I-167-a and I-167-b. The racemate was separated with chiral SFC (Method D) to afford the first eluting fraction (I-167-a) and second eluting fraction (I-167-b).

I-167-a: MS(ES): m/z: 516.17 [M+H]⁺, ¹H NMR (DMSO-d₆, 400 MHz): δ 13.44 (s, 1H), 8.48 (s, 1H), 8.15 (s, 1H), 8.05 (s, 1H), 8.00-7.97 (m, 2H), 7.72-7.69 (m, 1H), 7.00-6.63 (bs, 1H), 4.48-4.47 (m, 1H), 4.23 (s, 1H), 3.89-3.88 (d, J 7.6 Hz, 1H), 3.65 (s, 1H), 3.59 (s, 3H), 2.95 (s, 3H), 2.84-2.81 (m, 2H), 2.08-2.05 (m, 1H), 1.81-1.78 (m, 1H).

I-167-b: MS(ES): m/z: 516.17 [M+H]⁺, ¹H NMR (DMSO-d₆, 400 MHz): δ 13.44 (s, 1H), 8.47 (s, 1H), 8.168 (s, 1H), 8.05 (s, 1H), 8.00-7.97 (m, 2H), 7.79-7.69 (m, 1H), 7.00-6.63 (bs, 1H), 4.49-4.47 (m, 1H), 4.24 (s, 1H), 3.91-3.89 (d, J 7.6 Hz, 1H), 3.65 (s, 1H), 3.60 (s, 3H), 2.95 (s, 3H), 2.84-2.81 (m, 2H), 2.08-2.05 (m, 1H), 1.81-1.78 (m, 1H).

### Example 168: 1-(1-(1H-pyrazolo[3,4-b]pyridin-5-yl)-2-oxabicyclo[2.2.2]octan-4-yl)-1-methyl-3-(1-methyl-2-oxo-5-(trifluoromethyl)-1,2-dihydropyridin-3-yl)urea

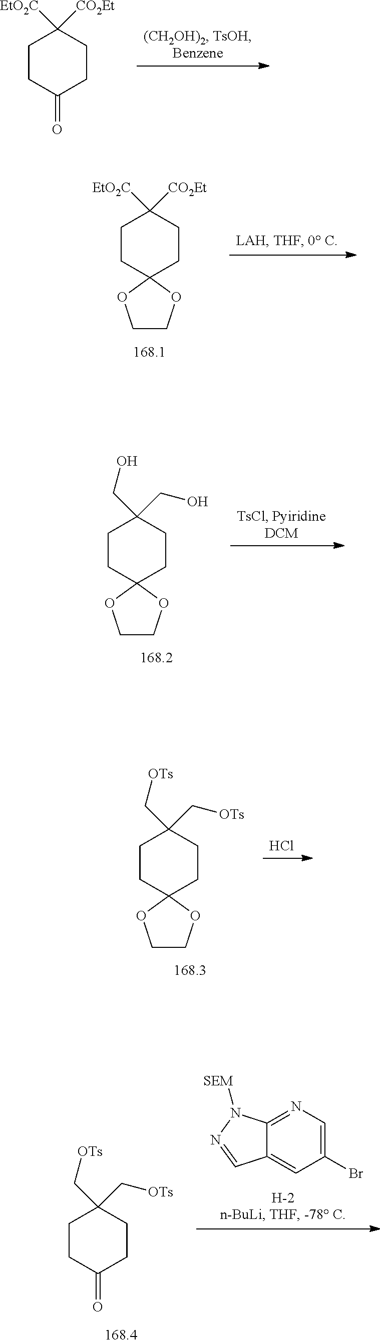

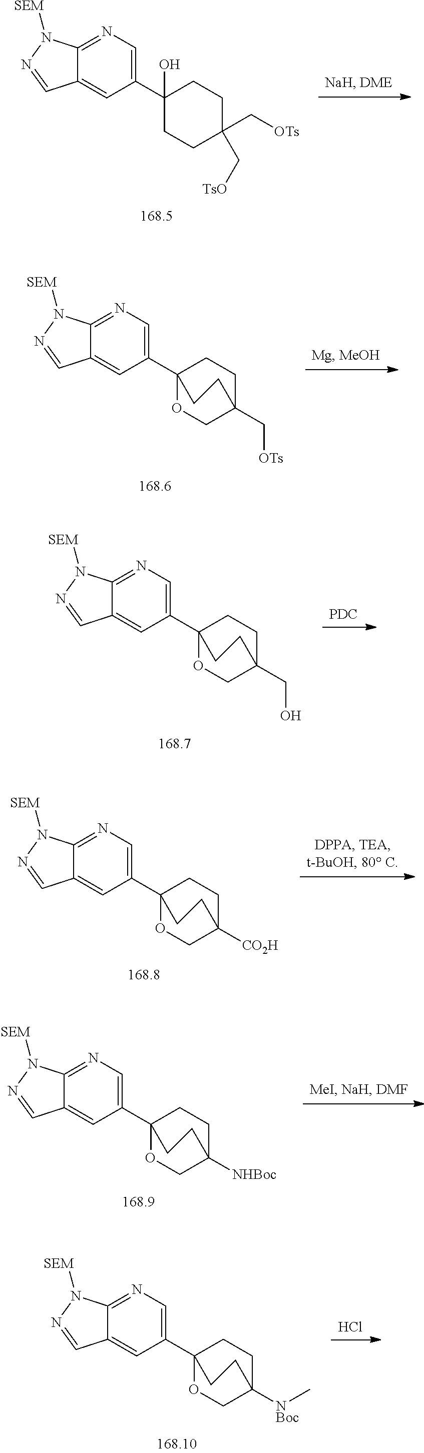

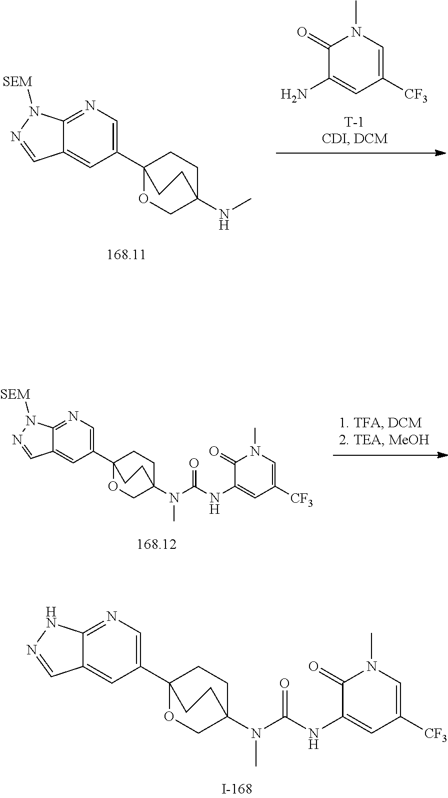

Synthesis of compound 168.1. To a stirred solution of diethyl 4-oxocyclohexane-1,1-dicarboxylate (38 g, 157.02 mmol, 1.0 equiv) in benzene (140 mL) was added 1,2-ethanediol (19.47 g, 314.03 mmol, 2 equiv) and p-toluene sulfonic acid (2.7 g, 15.70 mmol, 0.1 equiv). The reaction mixture was stirred at room temperature for 48 h. It was added to water, basified with saturated solution of sodium bicarbonate, and extracted with ethyl acetate. The combined organic layers were washed with brine, dried over anhydrous sodium sulfate, filtered, and concentrated under reduced pressure to afford 168.1 (40 g, 59%). MS(ES): m/z 287.14 [M+H]⁺.

Synthesis of compound 168.2. To a solution of 168.1 (40 g, 139.86 mmol, 1.0 equiv) in THE (400 mL) at 0° C. was added lithium aluminum hydride (12.3 g, 321.67 mmol, 2.3 equiv) in portions for 30 min. The reaction mixture was stirred at room temperature for 2 h. It was cooled to 0° C. and quenched with 15% sodium hydroxide solution. The reaction mixture was extracted with ethyl acetate. The combined organic layers were washed with brine, dried over anhydrous sodium sulfate, filtered, and concentrated under reduced pressure. The residue was purified by flash column chromatography on silica gel (55% ethyl acetate in hexane) to afford 168.2 (20 g, 71%). MS (ES): m/z 203.12 [M+H]⁺.

Synthesis of compound 168.3. To a stirred solution of 168.2 (20 g, 99.00 mmol, 1.0 equiv) in dry pyridine (140 mL) was added 4-toluenesulfonyl chloride (56.73 g, 297.02 mmol, 3.0 equiv) at 0° C. The reaction mixture was stirred at room temperature for 16 h. It was quenched with citric acid solution and extracted with ethyl acetate. The combined organic layers were washed with brine, dried over anhydrous sodium sulfate, filtered, and concentrated under reduced pressure. This residue was triturated with ethanol to afford 168.3 (30 g, 59%). MS(ES): m/z 511.14 [M+H]⁺.

Synthesis of compound 168.4. To a solution of 168.3 (30 g, 58.82 mmol, 1.0 equiv) in DCM (200.0 mL) at 0° C. was added 4 M hydrochloric acid in dioxane (100.0 mL) dropwise and stirred at room temperature for 2 h. It was concentrated under reduced pressure to afford 168.4 (20.0 g, 73%). MS (ES): m/z 467.11 [M+H]⁺.

Synthesis of compound 168.5. To a solution of 168.4 (20.0 g, 42.91 mmol, 1.0 equiv) and H-2 (14.03 g, 42.91 mmol, 1.0 equiv) in THE (200.0 mL) at −78° C. was added n-butyl lithium (2.5 M in hexane, 42.91 mL, 107.29 mmol, 2.5 equiv) and stirred for 5 h. It was poured into ice-water and extracted with ethyl acetate. The combined organic layers were washed with brine, dried over anhydrous sodium sulfate, filtered, and concentrated under reduced pressure. The residue was purified by flash column chromatography on silica gel (15.0% ethyl acetate in hexane) to afford 168.5 (9.0 g, 29%). MS (ES): m/z 716.24 [M+H]⁺.

Synthesis of compound 168.6. To a solution of 168.5 (9.0 g, 12.58 mmol, 1.0 equiv) in 1,1-dimethoxymethane (100.0 mL) at 0° C. was added sodium hydride (1.5 g, 37.76 mmol, 3.0 equiv) in portions. The reaction mixture was stirred at room temperature for 5 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (10.0% ethyl acetate in hexane) to afford 168.6 (6.0 g, 88%). MS(ES): m/z 544.22 [M+H]⁺.

Synthesis of compound 168.7. To a solution of 168.6 (6.0 g, 11.04 mmol, 1.0 equiv) in methanol (50.0 mL) at room temperature was added Mg powder (1.32 g, 55.24 mmol, 5.0 equiv). The reaction mixture was stirred at room temperature for 5 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (25.0% ethyl acetate in hexane) to afford 168.7 (3.50 g, 81%). MS(ES): m/z 390.21 [M+H]⁺.

Synthesis of compound 168.8. To a solution of 168.7 (3.5 g, 8.99 mmol, 1.0 equiv) in DCM (25.0 mL) at room temperature was added pyridinium dichromate (6.76 g, 17.99 mmol, 2.0 equiv). The reaction mixture was stirred at room temperature for 4 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (25.0% ethyl acetate in hexane) to afford 168.8 (2.50 g, 69%). MS(ES): m/z 404.19 [M+H]⁺.

Synthesis of compound 168.9. To a solution of 168.8 (2.5 g, 6.20 mmol, 1.0 equiv) in tert-butanol (15.0 mL) at room temperature was added triethylamine (1.72 mL, 12.40 mmol, 2.0 equiv) and diphenyl phosphoryl azide (3.4 g, 12.40 mmol, 2.0 equiv). The reaction mixture was stirred at 100° C. for 14 h. It was poured into ice water and extracted with ethyl acetate. The combined organic layers were washed with brine, dried over anhydrous sodium sulfate, filtered, and concentrated under reduced pressure. The residue was purified by flash column chromatography on silica gel (55.0% ethyl acetate in hexane) to afford 168.9 (1.0 g, 34%). MS(ES): m/z 475.21 [M+H]⁺.

Synthesis of compound 168.10. To a solution of 168.9 (1.0 g, 2.10 mmol, 1.0 equiv) in DMF (5.0 mL) at 0° C. was added sodium hydride (0.253 g, 6.32 mmol, 3.0 equiv). The reaction mixture was stirred for 30 min. To the mixture was added methyl iodide (0.449 g, 3.16 mmol, 1.5 equiv) and stirred at room temperature for 30 min. It was poured into ice water and extracted with ethyl acetate. The combined organic layers were washed with brine, dried over anhydrous sodium sulfate, filtered, and concentrated under reduced pressure. The residue was purified by flash column chromatography on silica gel (40.0% ethyl acetate in hexane) to afford 168.10 (0.60 g, 58%). MS(ES): m/z 489.28 [M+H]⁺.

Synthesis of compound 168.11. To a stirred solution of 168.10 (0.6 g, 1.22 mmol, 1.0 equiv) in DCM (5.0 mL) was added 4 M hydrochloric acid in dioxane (5.0 mL) and stirred at room temperature for 2 h. It was concentrated under reduced pressure to afford 168.11 (0.4 g, 84%). MS (ES): m/z 389.23 [M+H]⁺.

Synthesis of compound 168.12. To a solution of 168.11 (0.197 g, 1.03 mmol, 1.0 equiv) and triethylamine (0.55 mL, 3.66 mmol, 4.0 equiv) in THE (1.5 mL) was added a solution of triphosgene (0.104 g, 0.412 mmol, 0.4 equiv) in THE (0.5 mL) at 0° C. and stirred for 15 min. To the mixture was added T-1 (0.40 g, 1.03 mmol, 1.0 equiv). The reaction mixture was stirred at room temperature for 1 h. It was transferred into water, stirred, and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.6% methanol in DCM) to afford 168.12 (0.10 g, 16%). MS(ES): m/z 607.26 [M+H]⁺.

Synthesis of compound I-168. Compound I-168 was prepared from compound 168.12 following the procedure described in the synthesis of I-11. The product was purified by flash column chromatography on silica gel (CombiFlash®, 3.5% methanol in DCM). MS(ES): m/z 477.30 [M+H]⁺, ¹H-NMR (400 MHz, DMSO-d₆): δ 13.56 (s, 1H), 8.79 (s, 1H), 8.17 (s, 1H), 8.09 (s, 1H), 7.92 (s, 1H), 7.06 (d, 1H), 6.83 (d, 1H), 4.32 (m, 1H), 3.58 (s, 3H), 2.96 (s, 3H), 2.25 (m, 2H), 2.03-1.98 (m, 2H), 1.61-1.49 (m, 3H), 1.31-1.39 (m, 2H).

### Example 170: trans-1-(4-(7-methoxy-5H-pyrrolo[2,3-b]pyrazin-2-yl)cyclohexyl)-1-methyl-3-(1-methyl-2-oxo-5-(trifluoromethyl)-1,2-dihydropyridin-3-yl)urea

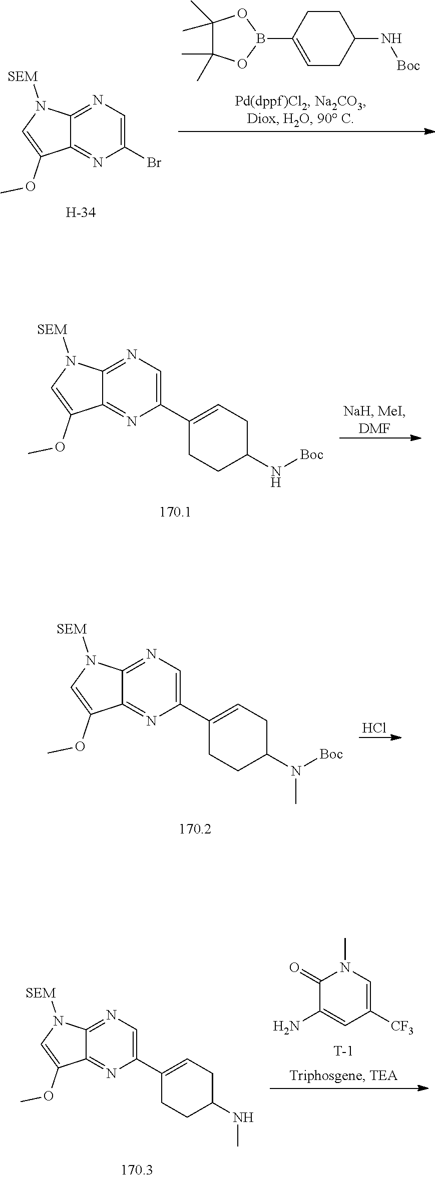

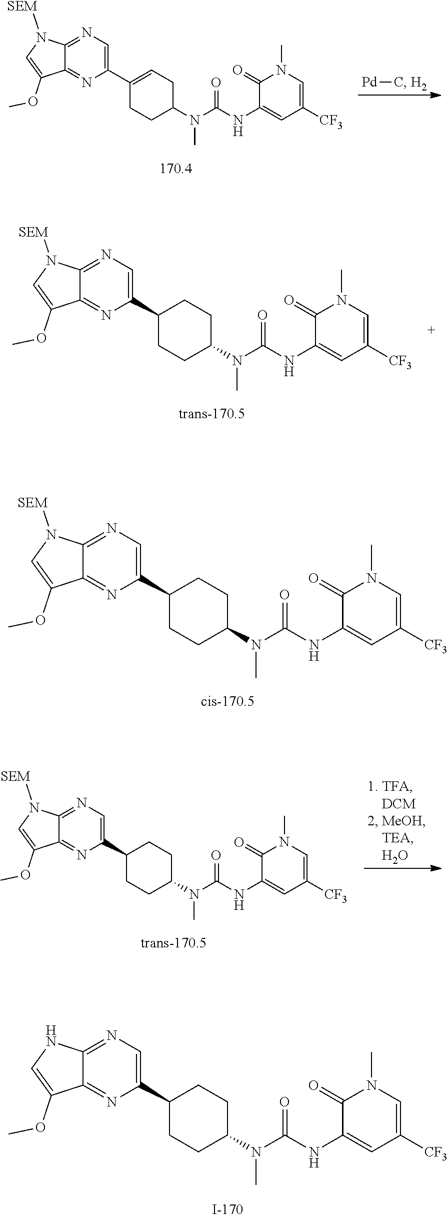

Synthesis of compound 170.1. To a solution of H-34 (0.6 g, 1.67 mmol, 1.0 equiv) and tert-butyl (4-(4,4,5,5-tetramethyl-1,3,2-dioxaborolan-2-yl)cyclohex-3-en-1-yl)carbamate (0.81 g, 2.51 mmol, 1.5 equiv) in 1,4-dioxane:water (8:2, 10 mL) was added sodium carbonate (0.532 g, 5.02 mmol, 3.0 equiv). The reaction mixture was degassed by bubbling through a stream of argon for 30 min. [1,1′-Bis(diphenylphosphino)ferrocene]dichloropalladium(II) (0.122 mL, 0.167 mmol, 0.1 equiv) was added and the reaction mixture was stirred at 90° C. for 1 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 21% ethyl acetate in n-hexane) to afford 170.1 (0.41 g, 527%). MS(ES): m/z 475.64 [M+H]⁺.

Synthesis of compound 170.2. To a solution of 170.1 (0.41 g, 0.863 mmol, 1.0 equiv) in DMF (10 mL) was added sodium hydride (0.086 g, 2.1 mmol, 2.5 equiv) at 0° C. and stirred for 30 min. To the mixture was added methyl iodide (0.184 g, 1.29 mmol, 1.5 equiv) and the reaction mixture was stirred at room temperature. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 12% ethyl acetate in n-hexane) to afford 170.2 (0.22 g, 52%). MS(ES): m/z 489.64 [M+H]⁺.

Synthesis of compound 170.3. To a solution of 170.2 (0.22 g, 45 mmol, 1.0 equiv) in DCM (6.0 mL) was added 4 M hydrochloric acid in dioxane (2.2 mL) at 0° C. It was concentrated under reduced pressure to afford 170.3 (0.17 g, 97%). MS(ES): m/z 389.31 [M+H]⁺.

Synthesis of compound 170.4. Compound 170.4 was prepared from compound 170.3 and T-1 following the procedure described in the synthesis of I-9. The product was purified by flash column chromatography on silica gel (3.0% methanol in DCM). MS(ES): m/z 607.26 [M+H]⁺.

Synthesis of compound trans-170.5 and 1-170.5. A mixture of 170.4 (0.092 g, 0.151 mmol, 1.0 equiv), methanol (10 mL) and 10% Pd/C (0.046 g, 50% w/w) was stirred under hydrogen for 1 h. It was filtered through a pad of Celite® pad. The filtrate was concentrated under reduced pressure. The residue was purified by flash column chromatography on silica gel (30% ethyl acetate in hexane) to afford trans-170.5 (0.057 g, 62%) and cis-170.5 (0.018 g, 20%). MS(ES): m/z 609.31 [M+H]⁺.

Synthesis of compound I-170. Compound I-170 was prepared from compound trans-170.5 following the procedure described in the synthesis of I-11. The product was purified by flash column chromatography on silica gel (CombiFlash®, 3.2% methanol in DCM). MS(ES): m/z: 478.45 [M+H]⁺, ¹H NMR (DMSO-d₆, 400 MHz): δ 11.30 (s, 1H), 8.35 (s, 1H), 8.18 (s, 1H), 7.92 (s, 1H), 7.40 (s, 1H), 4.11 (bs, 1H), 3.87 (s, 3H), 3.60 (s, 3H), 2.91 (s, 3H), 2.83 (m, 1H), 1.97 (m, 2H), 1.79-1.74 (m, 6H).

### Example 171: cis-1-(4-(7-methoxy-5H-pyrrolo[2,3-b]pyrazin-2-yl)cyclohexyl)-1-methyl-3-(1-methyl-2-oxo-5-(trifluoromethyl)-1,2-dihydropyridin-3-yl)urea

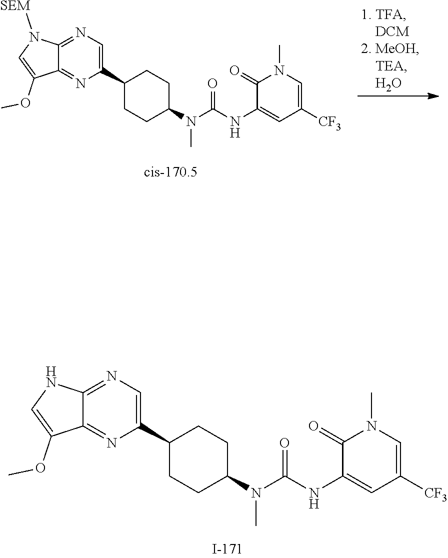 [SMILES: COc1cn(COCC[Si](C)(C)C)c2ncc([C@H]3CC[C@@H](N(C)C(=O)Nc4cc(C(F)(F)F)cn(C)c4=O)CC3)nc12 ; "1. TFA,     DCM2. MeOH,     TEA,    H2O" ; COc1c[nH]c2ncc([C@H]3CC[C@@H](N(C)C(=O)Nc4cc(C(F)(F)F)cn(C)c4=O)CC3)nc12]

Synthesis of compound I-171: Compound I-171 was prepared from compound cis-170.5 following the procedure described in the synthesis of I-11. The product was purified by flash column chromatography on silica gel (CombiFlash®, 3.2% methanol in DCM). MS(ES): m/z: 479.42 [M+H]⁺, ¹H NMR (DMSO-d₆, 400 MHz): δ 11.32 (s, 1H), 8.39 (s, 1H), 8.17 (s, 1H), 8.02 (s, 1H), 7.84 (s, 1H), 7.40 (s, 1H), 4.15 (bs, 1H), 3.91 (s, 3H), 3.58 (s, 3H), 3.27 (m, 1H), 2.74 (s, 3H), 1.89-1.84 (m, 6H), 1.48-1.46 (m, 2H).

### Example 172: (R)—N-(5-(3,3-difluoro-4-(1-methyl-3-(1-methyl-2-oxo-5-(trifluoromethyl)-1,2-dihydropyridin-3-yl)ureido)piperidin-1-yl)-1H-pyrrolo[2,3-b]pyridin-3-yl)acetamide and (S)—N-(5-(3,3-difluoro-4-(1-methyl-3-(1-methyl-2-oxo-5-(trifluoromethyl)-1,2-dihydropyridin-3-yl)ureido)piperidin-1-yl)-1H-pyrrolo[2,3-b]pyridin-3-yl)acetamide

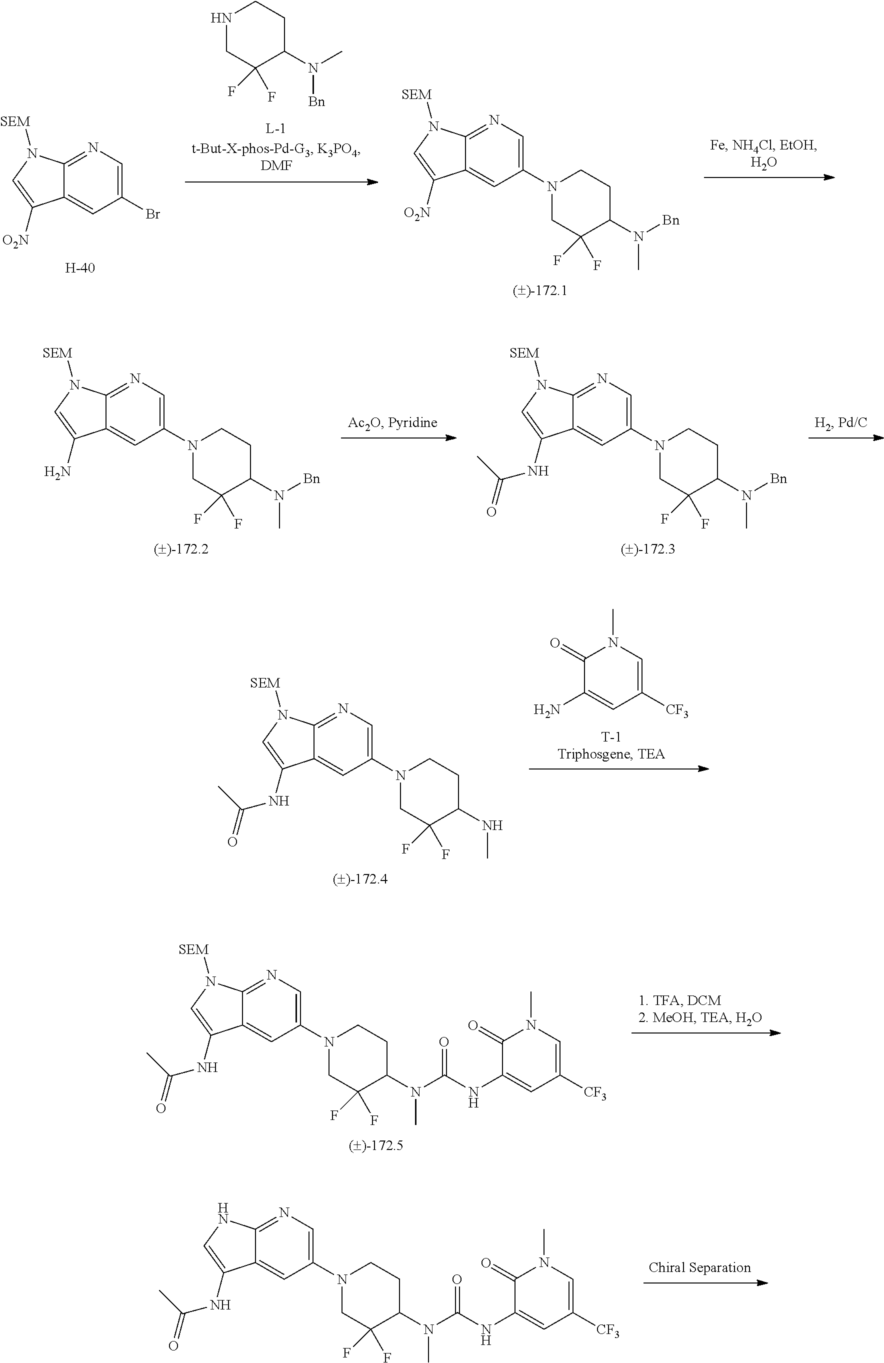

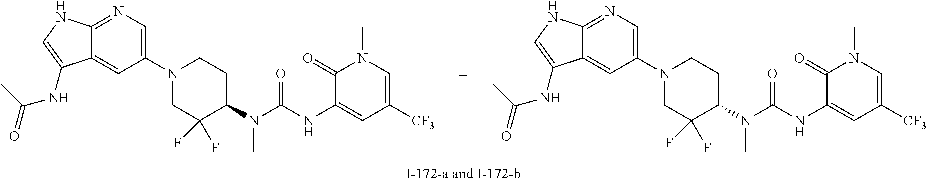 [SMILES: CC(=O)Nc1c[nH]c2ncc(N3CC[C@@H](N(C)C(=O)Nc4cc(C(F)(F)F)cn(C)c4=O)C(F)(F)C3)cc12 ; CC(=O)Nc1c[nH]c2ncc(N3CC[C@H](N(C)C(=O)Nc4cc(C(F)(F)F)cn(C)c4=O)C(F)(F)C3)cc12 ; "I-172-a and I-172-b"]

Synthesis of compound (±)-172.1. A mixture of H-40 (0.5 g, 1.34 mmol, 1.0 equiv), L-1 (0.387 mg, 1.61 mmol, 1.2 equiv), potassium phosphate (1.13 g, 5.37 mmol, 4.0 equiv), and t-butyl-X-phos-Pd-G₃ (0.32 g, 0.40 mmol, 0.3 equiv) in DMF (10 mL) was degassed by bubbling through a stream of argon for 10 min. The reaction mixture was stirred at room temperature for 16 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 1.8% methanol in DCM) to afford (±)-172.1 (0.135 g, 19%). MS(ES): m/z 532.50 [M+H]⁺.

Synthesis of compound (±)-172.2. To a solution of (±)-172.1 (0.54 g, 1.01 mmol, 1.0 equiv) in ethanol (10 mL) and water (3 mL) were added iron powder (0.284 g, 5.08 mmol, 5.0 equiv) and ammonium chloride (0.274 g, 5.08 mmol, 5.0 equiv). The reaction mixture was stirred at 80° C. for 1 h. It was transferred into water and extracted with ethyl acetate. The combined organic layers were washed with brine, dried over anhydrous sodium sulfate, filtered, and concentrated under reduced pressure to afford (±)-172.2 (0.520 g, 67%). MS(ES): m/z 502.28 [M+H]⁺.

Synthesis of compound (±)-172.3. To a solution of (±)-172.2 (0.52 g, 1.03 mmol, 1.0 equiv) in DCM (8 mL) were added acetic anhydride (0.105 g, 1.03 mmol, 1.0 equiv) and pyridine (4 mL). The reaction mixture was stirred at room temperature for 1 h. It was transferred into water and extracted with DCM. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford (±)-172.3 (0.325 g, 58%). MS(ES): m/z 544.35 [M+H]⁺.

Synthesis of compound (±)-172.4. A mixture of (±)-172.3 (0.325 g, 0.59 mmol, 1.0 equiv), methanol (8 mL) and 10% palladium on charcoal (0.2 g) was stirred under hydrogen atmosphere at room temperature for 2 h. It was filtered through a pad of Celite®. The filtrate was concentrated under reduced pressure to afford (±)-172.4 (0.20 g, 74%). MS(ES): m/z 454.27 [M+H]⁺.

Synthesis of compound (±)-172.5. Compound (±)-172.5 was prepared from compound (±)-172.4 and T-1 following the procedure described in the synthesis of I-9. The product was purified by flash column chromatography on silica gel (1.9% methanol in DCM). MS(ES): m/z 672.43 [M+H]⁺.

Synthesis of compound (±)-I-172. Compound (±)-I-172 was prepared from compound (±)-172.5 following the procedure described in the synthesis of I-11. The product was purified by flash column chromatography on silica gel (CombiFlash®, 3.3% methanol in DCM). MS(ES): m/z 542.23 [M+H]⁺.

I-172-a and I-172-b: The racemate was separated with chiral SFC (Method H) to afford first eluting fraction (I-172-a) and second eluting fraction (I-172-b).

I-172-a: MS(ES): m/z: 542.45 [M+H]⁺, ¹H NMR (DMSO-d₆, 400 MHz): δ 11.11 (s, 1H), 9.87 (s, 1H), 8.17-8.13 (m, 2H), 8.08 (s, 2H), 7.84-7.83 (d, J 2 Hz, 1H), 7.71-7.70 (d, J 2.4 Hz, 1H), 4.66-4-65 (m, 1H), 3.84-3.78 (t, J 10.8 Hz, 1H), 3.71-3.68 (d, J 12 Hz, 1H), 3.61 (s, 3H), 3.29-3.15 (m, 1H), 3.01 (s, 3H), 2.33-2.30 (m, 1H), 2.08 (s, 3H), 1.97-1.88 (m, 1H), 1.45-1.41 (m, 1H).

I-172-b: MS(ES): m/z: 542.40 [M+H]⁺, ¹H NMR (DMSO-d₆, 400 MHz): δ 11.11 (s, 1H), 9.87 (s, 1H), 8.17-8.13 (m, 2H), 8.08 (s, 2H), 7.84 (s, 1H), 7.70 (s, 1H), 4.86-4-76 (m, 1H), 3.83-3.71 (m, 1H), 3.71-3.68 (m, 1H), 3.61 (s, 3H), 3.29-3.15 (m, 1H), 3.01 (s, 3H), 2.33-2.30 (m, 1H), 2.08 (s, 3H), 1.97-1.88 (m, 1H), 1.45-1.41 (m, 1H).

### Example 173: (R)-1-(1-(1H-pyrazolo[3,4-b]pyridin-5-yl)piperidin-4-yl)-3-(5-(tert-butyl)-1-(tetrahydrofuran-3-yl)-1H-pyrazol-3-yl)-1-methylurea

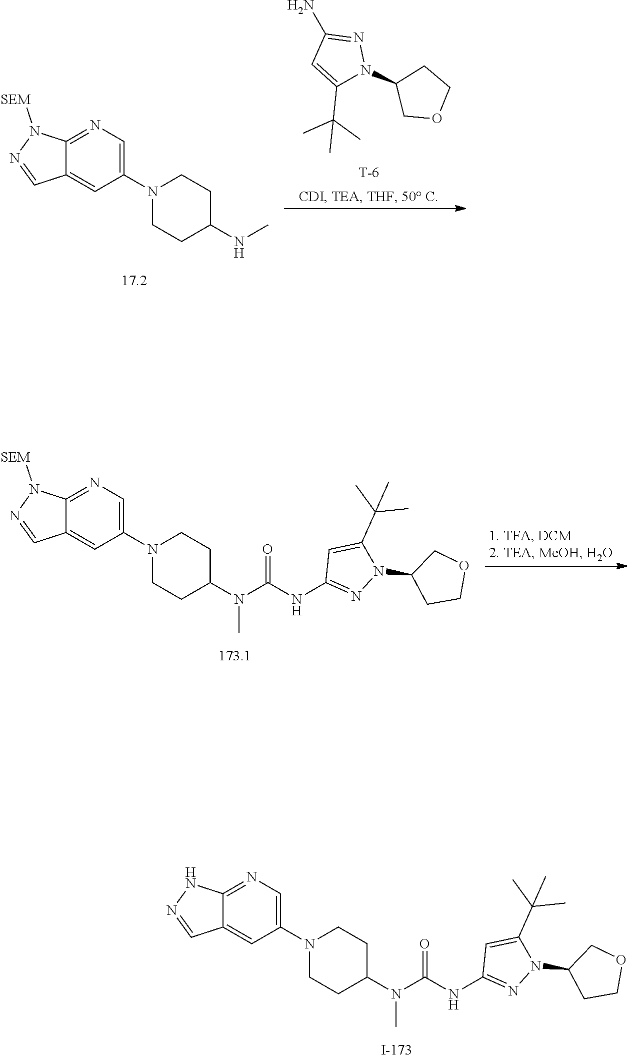

Synthesis of compound 173.1. To a solution of 17.2 (0.300 g, 1.56 mmol, 1.0 equiv) in THF (10 mL) were added T-6 (0.356 g, 1.56 mmol, 1.0 equiv), trimethylamine (2.36 g, 23.4 mmol, 15.0 equiv) and 1,1′-carbonyldiimidazole (1.77 g, 1.56 mmol, 1.0 equiv) at room temperature. The reaction mixture was stirred at 50° C. for 4 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 5% methanol in DCM) to afford 173.1 (0.089 g, 54%). MS(ES): m/z 597.69 [M+H]⁺.

Synthesis of I-173. Compound I-173 was prepared from compound 173.1 following the procedure described in the synthesis of I-11. The product was purified by flash column chromatography on silica gel (CombiFlash®, 3.2% methanol in DCM). MS(ES): m/z: 467.56 [M+H]⁺, ¹H NMR (DMSO-d₆, 400 MHz): δ 13.41 (s, 1H), 8.68 (s, 1H), 8.44-8.43 (d, J 2.4 Hz, 1H), 7.95-7.95 (d, J 1.2 Hz, 1H), 7.62-7.61 (d, J 2.4 Hz, 1H), 6.2 (s, 1H), 5.7 (s, 1H), 5.21-4.76 (m, 1H), 4.04-4.02 (m, 1H), 4.00-3.75 (m, 2H), 3.65-3.40 (m, 2H), 3.39-3.37 (m, 2H), 2.791 (s, 3H), 2.61-2.54 (m, 2H), 2.49-2.32 (m, 2H), 1.89-1.63 (m, 2H), 1.60-1.31 (m, 1H), 1.22 (s, 9H).

The following compound was prepared following the procedure of I-173:

| Cmpd. No. | Structure and Name | Analytical Data |
|---|---|---|
| I-174 | 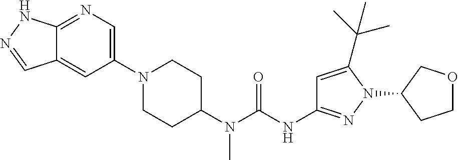 [SMILES: CN(C(=O)Nc1cc(C(C)(C)C)n([C@H]2CCOC2)n1)C1CCN(c2cnc3[nH]ncc3c2)CC1] (S)-1-(1-(1H-pyrazolo[3,4- b]pyridin-5-yl)piperidin-4-yl)- 3-(5-(tert-butyl)-1- (tetrahydrofuran-3-yl)-1H- pyrazol-3-yl)-1-methylurea | MS (ES): m/z 467.56 [M + H]⁺ ¹H NMR (DMSO-d₆, 400 MHz): δ 13. 38 (s, 1H), 8.71 (s, 1H), 8.45-8.44 (d, J = 2.4 Hz, 1H), 7.97 (s, 1H), 7.63 (d, J = 2.4 Hz, 1H), 6.13 (s, 1H), 5.76 (s, 1H), 4.17-4.04 (m, 1H), 4.03-4.00 (m, 2H), 3.87-3.76 (m, 2H), 3.66-3.63 (m, 2H), 2.80 (s, 3H), 2..33-2.28 (m, 1H), 2.26-2.19 (m, 2H), 2.09 (s,2H), 1.64-1.62 (m, 2H), 1.33 (s, 9H) |

### Example 175: 1-((3R,4R)-3-fluoro-1-(1H-pyrazolo[3,4-b]pyridin-5-yl)piperidin-4-yl)-1-methyl-3-(4-(trifluoromethyl)pyridin-2-yl)urea

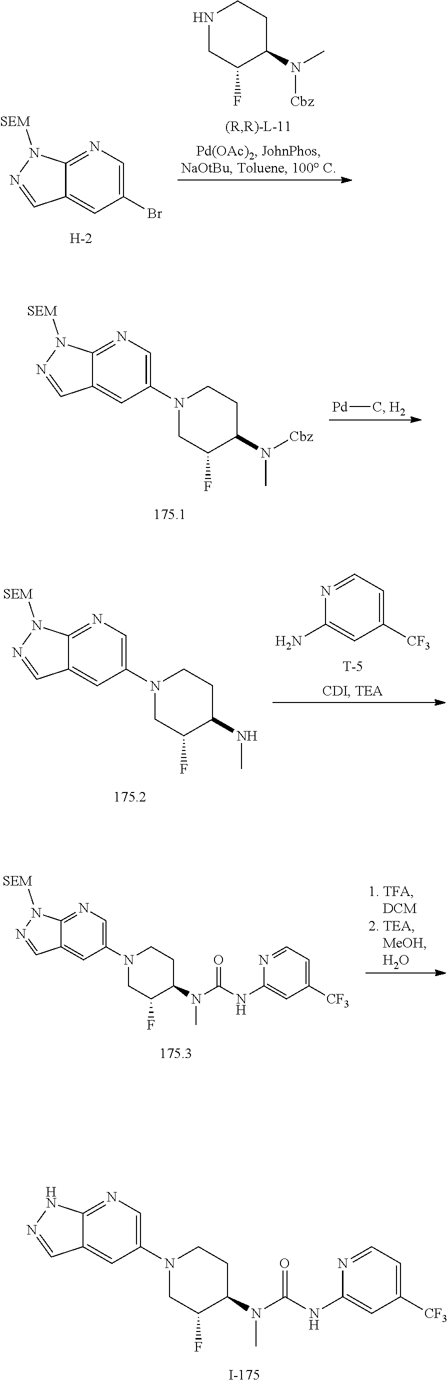

Synthesis of compound 175.1. A mixture of (R,R)-L-11 (0.6 g, 1.981 mmol, 1.0 equiv), H-2 (0.652 g, 1.988 mmol, 1.0 equiv) and sodium tert-butoxide (0.572 g, 5.96 mmol, 3.0 equiv) in 1,4-dioxane (5 mL) was degassed by bubbling through a stream of argon for 10 min. (2-Biphenyl)di-tert-butylphosphine (0.118 g, 0.42 mmol, 0.2 equiv) and palladium acetate (0.045 g, 0.198 mmol, 0.1 equiv) was added and degassed for 5 min. The reaction mixture was stirred at 100° C. for 1 h. It was poured into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 25% ethyl acetate in hexane) to afford 175.1 (0.48 g, 48%). MS(ES): m/z: 514.1 [M+H]⁺.

Synthesis of compound 175.2. A mixture of compound 175.1 (0.48 g, 0.934 mmol, 1.0 equiv) and 10% palladium on carbon (0.10 g) in methanol (5 mL) was stirred under hydrogen atmosphere for 5 h. It was filtered through a pad of Celite® and rinsed with methanol. The filtrate was concentrated under reduced pressure to afford 175.2 (0.32 g, 91%). MS(ES): m/z 380.12 [M+H]⁺.

Synthesis of compound 175.3. To a solution of 175.2 (0.16 g, 0.422 mmol, 1.0 equiv) and T-5 (0.068 g, 0.422 mmol, 1.0 equiv) in THE (3.0 mL) at 0° C. was added triethylamine (0.24 mL, 1.688 mmol, 4.0 equiv) followed by 1,1′-carbonyldiimidazole (0.068 g, 0.422 mmol, 1.0 equiv). The reaction mixture was stirred at room temperature for 6 h. It was poured into ice water and extracted with ethyl acetate. The combined organic layers were washed with brine, dried over anhydrous sodium sulfate, filtered, and concentrated under reduced pressure. The residue was purified by column chromatography on silica gel (1.5% methanol in DCM) to afford 175.3 (0.135 g, 59%). MS(ES): m/z 568.1 [M+H]⁺.

Synthesis of I-175. Compound I-175 was prepared from compound 175.3 following the procedure described in the synthesis of I-11. The product was purified by flash column chromatography on silica gel (CombiFlash®, 3.5% methanol in DCM). MS(ES): m/z 438.5 [M+H]⁺, ¹H-NMR (400 MHz, DMSO-d₆): δ 13.44 (s, 1H), 9.54 (s, 1H), 8.55-8.54 (d, J=5.2 Hz, 1H), 8.50-8.49 (d, J=2.8 Hz, 1H) 8.18 (s, 1H), 7.99 (s, 1H), 7.74-7.73 (d, J=2.4 Hz, 1H) 7.35-7.33 (m, 1H), 5.04-4.83 (m, 1H), 4.35-4.89 (m, 1H), 3.92-4.05 (m, 1H), 3.59-3.67 (m, 1H), 3.00 (s, 3H), 2.87-2.82 (m, 2H), 2.02-1.97 (m, 1H), 1.89-1.79 (m, 1H).

The following compounds were prepared following the procedure of I-175:

| Cmpd. | | |
|---|---|---|
| No. | Structure and Name | Analytical Data |
| I-176 | 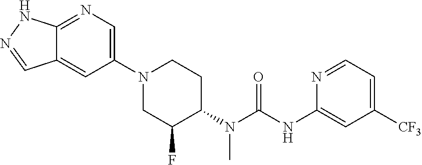 [SMILES: CN(C(=O)Nc1cc(C(F)(F)F)ccn1)[C@H]1CCN(c2cnc3[nH]ncc3c2)C[C@@H]1F] 1-((3S,4S)-3-fluoro-1-(1H- pyrazolo[3,4-b]pyridin-5- yl)piperidin-4-yl)-1-methyl-3-(4- (trifluoromethyl)pyridin-2-yl)urea | MS (ES): m/z 438.39 [M + H]⁺ ¹H-NMR (400 MHz, DMSO-d₆): δ 13.44 (s, 1H), 9.54 (s, 1H), 8.54-8.52 (d, J = 5.2 Hz, 1H), 8.50-8.49 (d, J = 2.4 Hz, 1H) 8.18 (s, 1H), 7.99 (s, 1H), 7.73-7.72 (d, J = 2.4 Hz, 1H) 7.35-7.33 (d, J = 4.8 Hz, 1H), 5.03-4.84 (m, 1H), 4.46-4.37 (m, 1H), 4.01-3.98 (m, 1H), 3.63-3.60 (m, 1H), 3.00 (s, 3H), 2.87-2.82 (m, 2H), 2.03-1.95 (m, 1H), 1.89-1.80 (m, 1H) |
| I-177 | 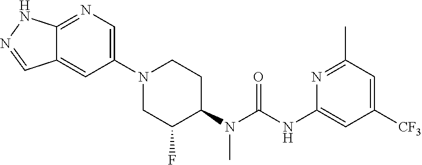 [SMILES: Cc1cc(C(F)(F)F)cc(NC(=O)N(C)[C@@H]2CCN(c3cnc4[nH]ncc4c3)C[C@H]2F)n1] 1-((3R,4R)-3-fluoro-1-(1H- pyrazolo[3,4-b]pyridin-5- yl)piperidin-4-yl)-1-methyl-3-(6- methyl-4-(trifluoromethyl)pyridin-2- yl)urea | MS (ES): m/z 452.31 [M + H]⁺ ¹H-NMR (400 MHz, DMSO-d₆): δ 13.43 (s, 1H), 9.42 (s, 1H), 8.49 (s, 1H), 7.98 (s, 2H), 7.72 (s, 1H), 7.21 (s, 1H), 4.98-4.85 (m, 1H), 4.40-4.39 (m, 1H), 3.99-3.97 (m, 1H), 3.62- 3.59 (m, 1H), 2.98 (s, 3H), 2.86-2.81 (m, 2H), 2.50 (s, 3H), 2.02-1.94 (m, 1H), 1.89-1.75 (m, 1H) |
| I-178 | 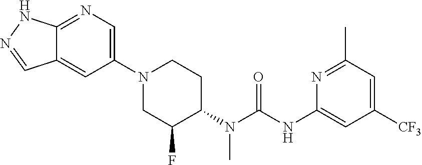 [SMILES: Cc1cc(C(F)(F)F)cc(NC(=O)N(C)[C@H]2CCN(c3cnc4[nH]ncc4c3)C[C@@H]2F)n1] 1-((35,4S)-3-fluoro-1-(1H- pyrazolo[3,4-b]pyridin-5- yl)piperidin-4-yl)-1-methyl-3-(6- methyl-4-(trifluoromethyl)pyridin-2- yl)urea | MS (ES): m/z 452.31 [M + H]⁺ ¹H-NMR (400 MHz, DMSO-d₆): δ 13.43 (s, 1H), 9.43 (bs, 1H), 8.50 (s, 1H), 7.99 (s, 2H), 7.73 (s, 1H), 7.22 (s, 1H), 5.02-4.83 (m, 1H), 4.45-4.36 (m, 1H), 4.00-3.97 (m, 1H), 3.62- 3.60 (m, 1H), 3.33 (s, 3H), 2.99 (s, 3H), 2.97- 2.82 (m, 2H), 2.09-1.95 (m, 1H), 1.89-1.79 (1H) |

### Example 179: N-(5-(3-(1-methyl-3-(1-methyl-2-oxo-5-(trifluoromethyl)-1,2-dihydropyridin-3-yl)ureido)bicyclo[1.1.1]pentan-1-yl)pyridin-2-yl)cyclopropanecarboxamnide

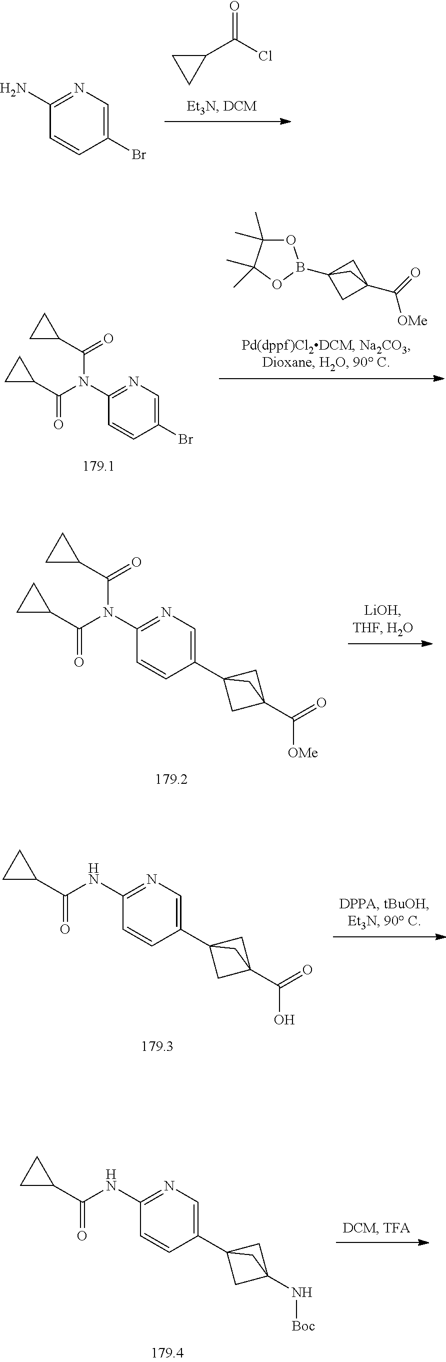

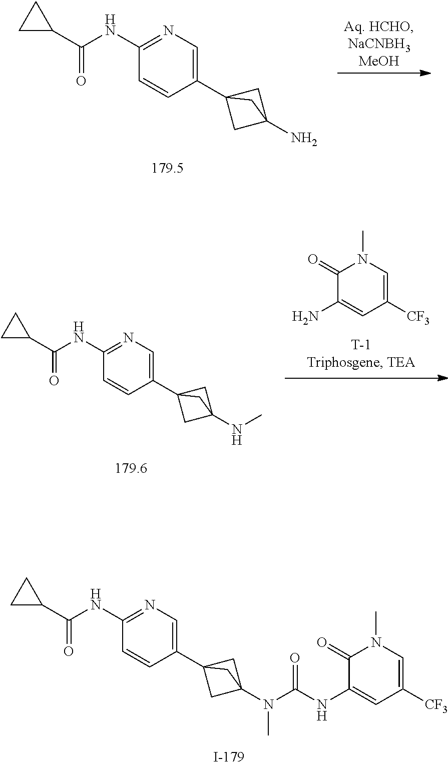

Synthesis of compound 179.1. To a solution of 5-bromopyridin-2-amine (5.0 g, 28.901 mmol, 1.0 equiv) in DCM at 0° C. was added triethylamine (12.51 mL, 86.705 mmol, 3.0 equiv) followed by dropwise addition of cyclopropanecarbonyl chloride (3.32 g, 31.77 mmol, 1.1 equiv). The reaction mixture was stirred at room temperature for 6 h. It was poured into water and extracted with DCM. The combined organic layers were washed with brine, dried over anhydrous sodium sulfate, filtered, and concentrated under reduced pressure. The residue was purified by flash column chromatography on silica gel (CombiFlash®, 20% ethyl acetate in hexane) to afford 179.1 (4.1 g, 47%). MS(ES): m/z: 309.1 [M+H]⁺ and 311.1 [M+H]⁺.

Synthesis of compound 179.2. A mixture of 179.1 (1.0 g, 3.236 mmol, 1.0 equiv) and methyl 3-(4,4,5,5-tetramethyl-1,3,2-dioxaborolan-2-yl)bicyclo[1.1.1]pentane-1-carboxylate (0.897 g, 3.559 mmol, 1.1 equiv) in 1,4-dioxane (10 mL) and water (0.1 mL) was added sodium carbonate (1.029 g, 9.708 mmol, 3.0 equiv). The reaction mixture was degassed by bubbling through a stream of argon for 10 min. Bis(diphenylphosphino)ferrocene]dichloropalladium(II) DCM complex (0.355 g, 0.486 mmol, 0.15 equiv) was added and the reaction mixture was stirred at 90° C. for 4 h. It was transferred into ice-water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 30% ethyl acetate in hexane) to afford 179.2 (0.235 g, 21%). MS(ES): m/z 355.1 [M+H]⁺.

Synthesis of compound 179.3. To a solution of 179.2 (0.235 g, 0.663 mmol, 1.0 equiv) in a THE (2 mL)/water (2 mL) mixture at 0° C. was added lithium hydroxide (0.08 g, 3.351 mmol, 5.0 equiv) and stirred reaction mixture at room temperature for 3 h. It was diluted with ethyl acetate and water. The aqueous layer was separated, acidified with 1 N hydrochloric acid to pH 2.5 and extracted with ethyl acetate. The combined organic layers were dried over anhydrous sodium sulfate, filtered, and concentrated under reduced pressure to afford 179.3 (0.18 g, yield: 99%). MS(ES): m/z 273.1 [M+H]⁺.

Synthesis of compound 179.4. To a solution of 179.3 (0.13 g, 0.661 mmol, 1.0 equiv) in t-butanol (2 mL) was added triethylamine (0.206 mL, 1.433 mmol, 3.0 equiv) followed by addition of diphenylphosphoryl azide (0.17 g, 0.621 mmol, 1.3 equiv). The reaction mixture was stirred at 90° C. for 24 h. It was cooled to room temperature and transferred into water. The mixture was extracted with ethyl acetate. The combined organic layers were dried over anhydrous sodium sulfate, filtered, and concentrated under reduced pressure. The residue was purified by column chromatography on silica gel (30% ethyl acetate in hexane) to afford 179.4 (0.115 g, yield: 71%). MS(ES): m/z 344.12 [M+H]⁺.

Synthesis of compound 179.5. To a solution of 179.4 (0.115 g, 0.335 mmol, 1.0 equiv) in DCM (2 mL) was added trifluoracetic acid (1.5 mL). The reaction mixture was stirred at room temperature for 1 h. It was transferred into saturated sodium bicarbonate solution and extracted with DCM. The combined organic layers were washed with brine, dried over anhydrous sodium sulfate, filtered, and concentrated under reduced pressure to afford 179.5 (0.075 g, yield: 93%). MS(ES): m/z 244.15 [M+H]⁺.

Synthesis of compound 179.6. To a solution of 179.5 (0.075 g, 0.308 mmol, 1.0 equiv) in methanol and formaldehyde (0.012 g, 0.370 mmol, 1.2 equiv) at 0° C. was added sodium cyanoborohydride (0.025 g, 0.401 mmol, 1.0 equiv). The reaction mixture was stirred at room temperature for 6 h. It was poured into water and extracted with DCM. The combined organic layers were washed with brine, dried over anhydrous sodium sulfate, filtered, and concentrated under reduced pressure. The residue was purified by flash column chromatography on silica gel (CombiFlash®, 35% ethyl acetate in hexane) to afford 179.6 (0.041 g, 52%). MS(ES): m/z: 258.12 [M+H]⁺.

Synthesis of I-179. Compound I-179 was prepared from compound 179.6 and T-1 following the procedure described in the synthesis of 1-9. The product was purified by column chromatography on silica gel (2.5% methanol in DCM). MS(ES): m/z 476.61 [M+H]⁺, ¹H-NMR (400 MHz, DMSO-d₆): δ10.55 (s, 1H), 8.19-8.18 (d, J=4.0 Hz, 1H), 8.05-8.04 (d, J=4.0 Hz, 2H), 7.94-7.92 (d, J=8.0 Hz, 2H), 7.44 (m, 1H), 3.60 (s, 3H), 2.89 (s, 3H), 2.77-2.74 (m, 2H), 1.95-1.90 (m, 2H), 1.68-1.64 (m, 2H), 1.25 (s, 1H), 0.78-0.76 (m, 4H).

### Example 180: (R)-1-(1-(1H-pyrazolo[3,4-b]pyridin-5-yl)piperidin-4-yl)-1-methyl-3-(1-(tetrahydrofuran-3-yl)-5-(trifluoromethyl)-1H-pyrazol-3-yl)urea

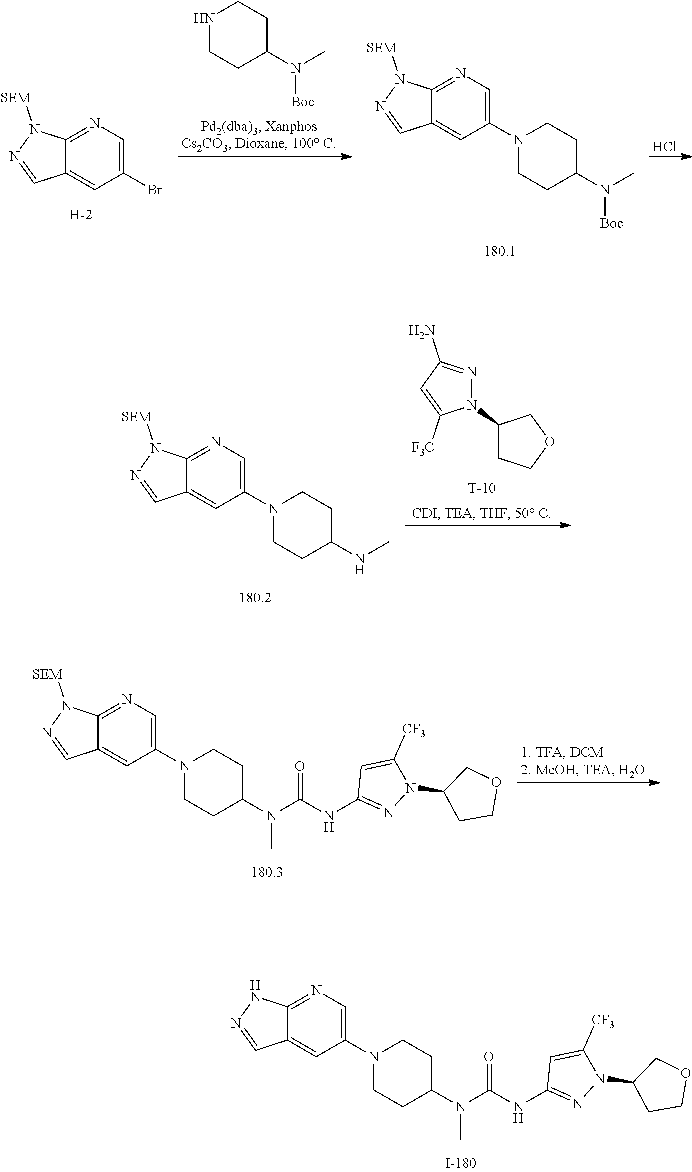

Synthesis of compound 180.1. A mixture of H-2 (3.1 g, 9.45 mmol, 1.0 equiv), tert-butyl methyl(piperidin-4-yl)carbamate (2.22 g, 10.39 mmol, 1.0 equiv) and cesium carbonate (7.67 g, 23.62 mmol, 2.5 equiv) in 1,4-dioxane (15 mL) was degassed by bubbling through a stream of argon for 10 min. 4,5-Bis(diphenylphosphino)-9,9-dimethylxanthene (0.819 g, 1.416 mmol, 0.15 equiv) and tris(dibenzylideneacetone)dipalladium(O) (0.129 g, 0.141 mmol, 0.015 equiv) were added. The reaction mixture was degassed for 15 min and stirred at 100° C. for 1.5 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 40% ethyl acetate in hexane) to afford 180.1 (1.6 g, 37%). MS(ES): m/z 462.2 [M+H]⁺.

Synthesis of compound 180.2. To a solution of 180.1 (1.6 g, 0.347 mmol, 1.0 equiv) in DCM (15 mL) at 0° C. was added 4 M hydrochloric acid in dioxane (1.0 mL). The reaction mixture was stirred at room temperature for 1 h. It was concentrated under reduced pressure to afford 180.2 (0.80 g, 64%). MS(ES): m/z 362.21 [M+H]⁺.

Synthesis of compound 180.3. To a solution of 180.2 (0.4 g, 1.10 mmol, 1.0 equiv) and T-10 (0.244 g, 1.10 mmol, 1.0 equiv) in THF (4.0 mL) at 0° C. was added triethylamine (0.63 mL, 4.42 mmol, 4 equiv) followed by 1,1′-carbonyldiimidazole (0.215 g, 1.33 mmol, 1.2 equiv). The reaction mixture was stirred at room temperature for 6 h. It was poured into ice water and extracted with ethyl acetate. The combined organic layers were washed with brine, dried over anhydrous sodium sulfate, filtered, and concentrated under reduced pressure. The residue was purified by column chromatography on silica gel (1.500 methanol in DCM) to afford 180.3 (0.138 g, 21%). MS(ES): m/z 609.21 [M+H]⁺.

Synthesis of compound I-180. Compound I-180 was prepared from compound 180.3 following the procedure described in the synthesis of I-11. The product was purified by flash column chromatography on silica gel (CombiFlash®, 2.50 methanol in DCM). MS(ES): m/z 479.52 [M+H]⁺, ¹H-NMR (400 z, DMSO-d₆): δ 13.37 (s, 1H), 9.25 (s, 1H), 8.45-8.44 (d, J=2.4 Hz, 1H), 7.97-7.96 (d, J=1.2 Hz, 1H), 7.64-7.63 (d, J=2.4 Hz, 1H), 6.88 (s, 1H), 5.04-5.02 (m, 1H), 4.22-4.16 (m, 1H), 4.06-4.01 (m, 2H), 3.87-3.78 (m, 2H), 3.67-3.64 (m, 2H), 2.83 (s, 3H), 2.79-2.76 (m, 2H), 2.45-2.39 (m, 1H), 2.38-2.23 (m, 1H), 1.95-1.87 (m, 2H), 1.67-1.64 (m, 2H).

The following compounds were prepared following the procedure of I-180:

| Cmpd. | | |
|---|---|---|
| No. | Structure and Name | Analytical Data |
| I-181 | 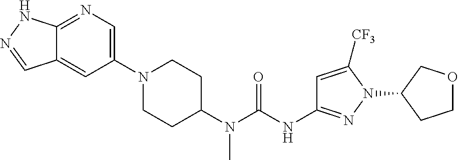 [SMILES: CN(C(=O)Nc1cc(C(F)(F)F)n([C@H]2CCOC2)n1)C1CCN(c2cnc3[nH]ncc3c2)CC1] (S)-1-(1-(1H-pyrazolo[3,4-b]pyridin-5- yl)piperidin-4-yl)-1-methyl-3-(1- (tetrahydrofuran-3-yl)-5- (trifluoromethyl)-1H-pyrazol-3-yl)urea | MS (ES): m/z 479.52 [M + H]⁺ ¹H-NMR (400 MHz, DMSO-d₆): δ 13.37 (s, 1H), 9.25 (s, 1H), 8.45-8.44 (d, J = 2.4 Hz, 1H), 7.97-7.96 (d, J = 1.2 Hz, 1H), 7.64-7.63 (d, J = 2.4 Hz, 1H), 6.88 (s, 1H), 5.05-5.02 (m, 1H), 4.19-4.18 (m, 1H), 4.08-4.01 (m, 2H), 3.87-3.78 (m, 2H), 3.67-3.64 (m, 2H), 2.83 (s, 3H), 2.79-2.76 (m, 2H), 2.45-2.39 (m, 1H), 2.38-2.23 (m, 1H), 1.93-1.89 (m, 2H), 1.67-1.64 (m, 2H) |
| I-183-aª and I-183-b^{b} | 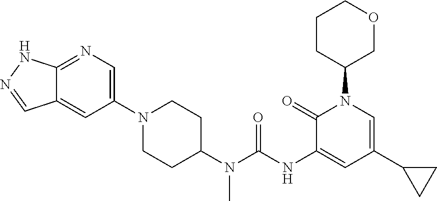 [SMILES: CN(C(=O)Nc1cc(C2CC2)cn([C@H]2CCCOC2)c1=O)C1CCN(c2cnc3[nH]ncc3c2)CC1] (S)- or (R)-1-(1-(1H-pyrazolo[3,4- b]pyridin-5-yl)piperidin-4-yl)-3-(5- cyclopropyl-2-oxo-1-(tetrahydro-2H- pyran-3-yl)-1,2-dihydropyridin-3-yl)-1- methylurea | MS (ES): m/z 492.57 [M + H]⁺ ¹H-NMR (400 MHz, DMSO-d₆): δ 13.37 (s, 1H), 8.45 (s, 1H), 8.23 (s, 1H), 7.96 (s, 1H), 7.86 (s, 1H), 7.63 (s, 1H), 7.37 (s, 1H), 6.24-6.20 (m, 1H), 5.94-5.88 (m, 1H), 4.79 (m, 1H), 4.20-4.17 (m, 1H), 3.83-3.77 (t, J = 12.0 Hz, 2H), 3.69-3.66 (d, J = 12.0 Hz, 2H), 3.59-3.54 (t, J = 12.0 Hz, 1H), 3.44- 3.39 (d, J = 8.0 Hz, 1H), 2.84 (s, 3H), 2.80- 2.74 (t, J = 12.0 Hz, 2H), 2.05-2.03 (m, 1H), 1.92-1.90 (m, 3H), 1.80-1.74 (m, 3H), 1.69-1.67 (m, 4H) |

^{a}Separated by chiral SFC (Method E); first eluting isomer;
^{b}Separated by chiral SFC (Method E); second eluting isomer.

### Example 184: 1-(1-(3-((1R,2R)-2-hydroxycyclobutoxy)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea and 1-(1-(3-((1S,2S)-2-hydroxycyclobutoxy)-1H-pyrazolo[3,4-b]pyridin-5-yl)piperidin-4-yl)-1-methyl-3-(1-methyl-2-oxo-5-(trifluoromethyl)-1,2-dihydropyridin-3-yl)urea

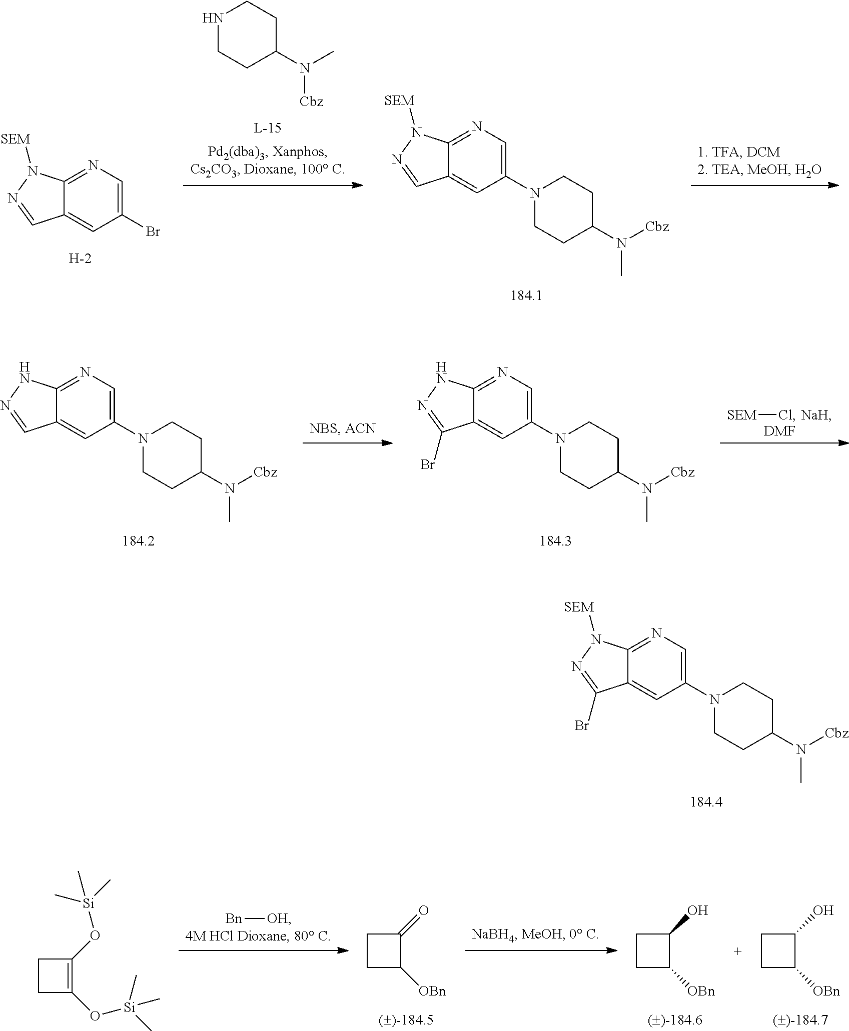

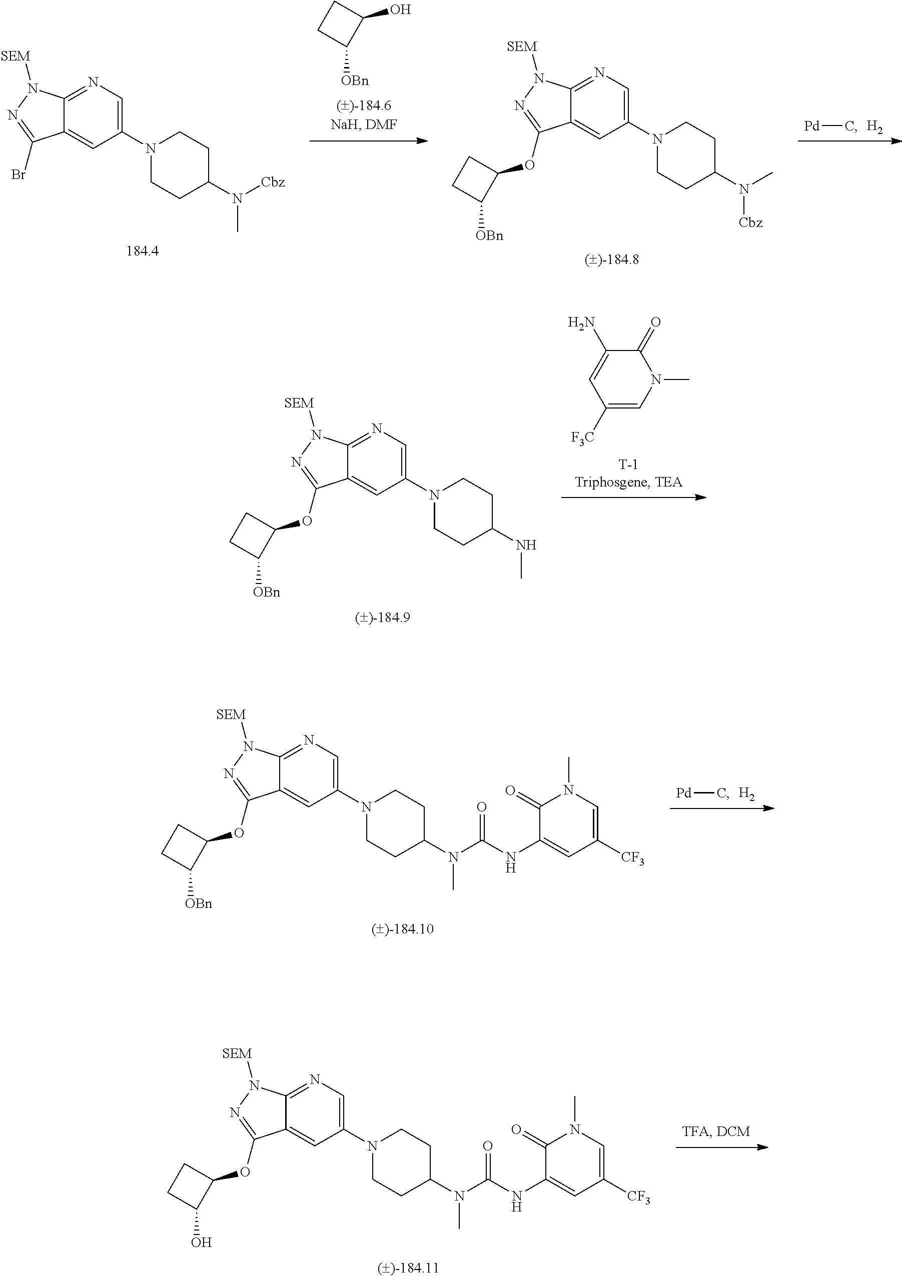

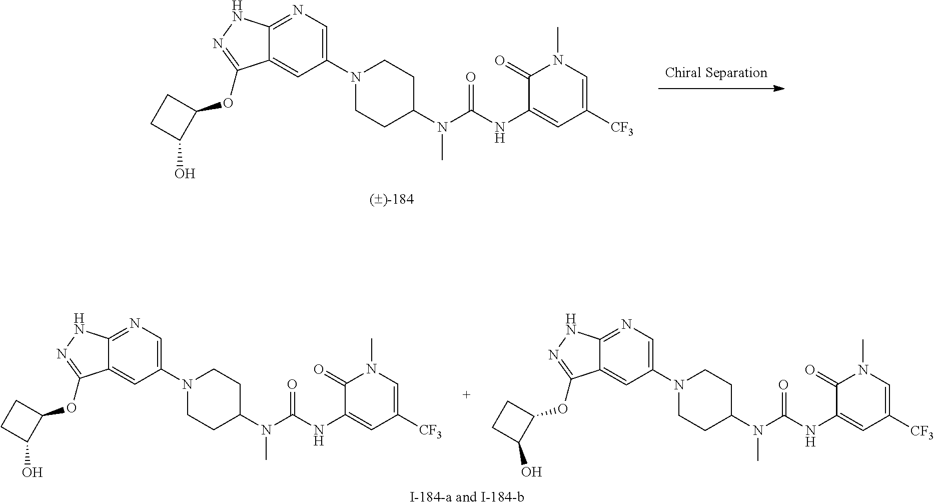

Synthesis of compound 184.1. Compound 184.1 was prepared from H-2 and L-15, following the procedure described in the synthesis of 180.1. The product was purified by flash column chromatography on silica gel (CombiFlash®, 30% ethyl acetate in hexane). MS(ES): m/z: 496.57 [M+H]⁺.

Synthesis of compound 184.2. Compound 184.2 was prepared from 184.1 following the procedure described in the synthesis of I-11. The product was purified by flash column chromatography on silica gel (CombiFlash®, 3.2% methanol in DCM). MS(ES): m/z: 366.42 [M+H]⁺.

Synthesis of compound 184.3. To a solution of 184.2 (1.8 g, 4.931 mmol, 1.0 equiv) in acetonitrile (5 mL) was added N-bromosuccinimide (1.14 g, 6.41 mmol, 1.3 equiv) at 0° C. and stirred at room temperature for 8 h. It was poured into saturated sodium bicarbonate solution and extracted with ethyl acetate. The combined organic layers were washed with brine, dried over anhydrous sodium sulfate, filtered, and concentrated under reduced pressure. The residue was purified by flash column chromatography on silica gel (CombiFlash®, 15% ethyl acetate in hexane) to afford 184.3 (1.45 g, 66%). MS(ES): m/z: 444.35 and 446.35 [M+H]⁺.

Synthesis of compound 184.4. To a solution of 184.3 (1.45 g, 3.264 mmol, 1.0 equiv) in DMF (15 mL) at 0° C. was added sodium hydride (60% in mineral oil, 1.08 g, 3.591 mmol, 1.2 equiv) in portions and stirred for 15 min. 2-(Trimethylsilyl)ethoxymethyl chloride (0.59 g, 3.592 mmol, 1.2 equiv) was added and the mixture was stirred at room temperature for 2 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford 184.4 (1.2 g, 62%). MS(ES): m/z 574.32 and 576.32 [M+1]⁺.

Synthesis of compound (±)-184.5. To a solution of 1,2-bis((trimethylsilyl)oxy)cyclobut-1-ene (5.0 g, 21.73 mmol, 1.0 equiv) in 4 M hydrochloric acid 1,4-dioxane (50 mL) at 0° C. was added dropwise benzyl alcohol (2.58 g, 23.91 mmol, 1.1 equiv). The mixture was stirred at 80° C. for 6 h. It was cooled to rt, transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford (±)-184.5 (2.8 g, 74%). MS(ES): m/z 177.1[M+1]⁺.

Synthesis of compound (±)-184.6 and (±)-184.7. To a solution of (±)-184.5 (2.8 g, 15.90 mmol, 1.0 equiv) in methanol (15 mL) at 0° C. was added sodium borohydride (0.725 g, 19.09 mmol, 1.2 equiv) in portions and stirred at room temperature for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 16-19% ethyl acetate in hexane) to afford (±)-184.6 (0.91 g, 62%) and (±)-184.7 (0.82 g, 33%). MS(ES): m/z 179.15 [M+1]⁺.

Synthesis of compound (±)-184.8. To a solution of 184.4 (0.372 g, 2.09 mmol, 1.0 equiv) in DMF (12 mL) at 0° C. was added sodium hydride (60% in mineral oil, 0.101 g, 2.507 mmol, 1.2 equiv) in portions and stirred for 15 min followed by addition of (±)-184.6 (1.2 g, 2.090 mmol, 1.0 equiv) in DMF (1 mL). The reaction mixture was stirred at room temperature for 3 h. It was transferred into water and extracted with ethyl acetate. The combined organic layers were washed with brine, dried over anhydrous sodium sulfate, filtered, and concentrated under reduced pressure. The residue was purified by flash column chromatography on silica gel (CombiFlash®, 10% ethyl acetate in hexane) to afford (±)-184.8 (0.65 g, 46%). MS(ES): m/z 672.68 [M+1]⁺.

Synthesis of compound (±)-184.9. A mixture of (±)-184.8 (0.65 g, 0.967 mmol, 1.0 equiv), methanol (7 mL) and 10% palladium on charcoal (0.2 g) was stirred under hydrogen (1 atm) at room temperature for 6 h. It was filtered through a pad of Celite®. The filtrate was concentrated under reduced pressure to afford (±)-184.9 (0.52 g, yield: 99%). MS(ES): m/z 536.62 [M+H]⁺.

Synthesis of compound (±)-184.10. Compound (±)-184.10 was prepared from compound (±)-184.9 and T-1 following the procedure described in the synthesis of I-9. The product was purified by flash column chromatography on silica gel (CombiFlash®, 32% ethyl acetate in hexane). MS(ES): m/z 756.78 [M+H]⁺.

Synthesis of compound (±)-184.11. A mixture of (±)-184.10 (0.32 g, 0.423 mmol, 1.0 equiv), methanol (3 mL) and 10% palladium on charcoal (0.1 g) was stirred under hydrogen (1 atm) at room temperature for 6 h. It was filtered through a pad of Celite®. The filtrate was concentrated under reduced pressure to afford (±)-184.11 (0.155 g, yield: 55%). MS(ES): m/z 666.41 [M+H]⁺.

Synthesis of compound (±)-184. Compound (±)-184 was prepared from compound (±)-184.11 following the procedure described in the synthesis of I-11. The product was purified by flash column chromatography on silica gel (CombiFlash®, 3.2% methanol in DCM). MS(ES): m/z 536.60 [M+H]⁺.

I-184-a and I-184-b: The racemate was separated by chiral SFC (Method D) to afford first eluting fraction (I-184-a) and second eluting fraction (I-184-b).

I-184-a: MS(ES): m/z: 536.59 [M+H]⁺, ¹H-NMR (400 MHz, DMSO-d₆): δ 13.36 (s, 1H), 8.34 (s, 1H), 8.20 (s, 1H), 8.19 (s, 1H), 8.03 (s, 1H), 7.92 (s, 1H), 5.69 (m, 1H), 5.06-5.03 (m, 1H), 4.64-4.57 (m, 1H), 4.20-4.17 (m, 2H), 3.55 (s, 3H), 3.37-3.34 (m, 1H), 3.31-3.26 (m, 2H), 3.02-2.87 (m, 2H), 2.94 (s, 3H), 2.22-2.21 (m, 1H), 2.10-2.08 (m, 1H), 1.96-1.88 (m, 2H), 1.68-1.65 (m, 2H).

I-184-b: MS(ES): m/z 536.60 [M+H]⁺, ¹H-NMR (400 MHz, DMSO-d₆): δ 13.36 (s, 1H), 8.32 (s, 1H), 8.19 (s, 1H), 8.18 (s, 1H), 8.02 (s, 1H), 7.91 (s, 1H), 5.69-5.65 (m, 1H), 5.05-5.02 (m, 1H), 4.64-4.57 (m, 1H), 4.18-4.10 (m, 2H), 3.59 (s, 3H), 3.37-3.34 (m, 1H), 3.31-3.26 (m, 2H), 3.02-2.87 (m, 2H), 2.94 (s, 3H), 2.25-2.19 (m, 1H), 2.09-2.07 (m, 1H), 1.96-1.89 (m, 2H), 1.68-1.65 (m, 2H).

### JAK2 Binding Assay

JAK2 (JH1 domain-catalytic, Y1007F, Y1008F) kinase was expressed as N-terminal fusion to the DNA binding domain of NFkB in transiently transfected HEK293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mmol/L DTT) to remove unbound ligand and to reduce nonspecific phage binding. Binding reactions were assembled by combining kinases, liganded affinity beads, and test compounds in 1× binding buffer (1× PBS, 0.05% Tween 20, 0.1% BSA, 1 mmol/L DTT). Test compound was prepared as 111× stocks in 100% DMSO and directly diluted into the assay wells. All reactions were performed in polypropylene 384-well plates in a final volume of 0.02 mL. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μmol/L non-biotinylated affinity ligand) and incubated at room temperature with shaking for 30 minutes. The kinase concentration in the eluate was measured by qPCR.

Results of the JAK2 JH1 Domain Binding Assay described above are presented in Table 2. Compounds denoted as “A” had a K_{d}<10 nM; compounds denoted as “B” had a K_{d}≥10 nM and <50 nM; compounds denoted as “C” had a K_{d}≥50 nM and <1 μM; compounds denoted as “D” had a K_{d}≥1 μM and <5 μM; and compounds denoted as “E” had a K_{d}≥5 μM.

TABLE 2

| Compound | JAK2 K_{d} |
|---|---|
| I-1 | D |
| I-2 | C |
| I-3 | C |
| I-4 | C |
| I-5 | C |
| I-6 | C |
| I-7 | D |
| I-8 | B |
| I-9 | D |
| I-10 | B |
| I-11 | A |
| I-12 | A |
| I-13 | A |
| I-14 | A |
| I-15 | A |
| I-16 | A |
| I-17 | A |
| I-18 | A |
| I-19 | B |
| I-20 | A |
| I-21 | A |
| I-22-a | B |
| I-22-b | B |
| I-23 | A |
| I-24 | A |
| I-25 | A |
| I-26 | A |
| I-27-a | A |
| I-27-b | A |
| I-28 | A |
| I-29-a | A |
| I-29-b | A |
| I-30 | A |
| I-31 | A |
| I-33-a | A |
| I-33-b | A |
| I-34-a | A |
| I-34-b | A |
| I-35-a | A |
| I-35-b | A |
| I-36-a | A |
| I-36-b | A |
| I-37-a | A |
| I-37-b | A |
| I-38 | B |
| I-39 | A |
| I-40 | A |
| I-41-a | D |
| I-41-b | C |
| I-42 | A |
| I-43-a | B |
| I-43-b | B |
| I-44-a | B |
| I-44-b | B |
| I-45-a | A |
| I-45-b | B |
| I-46 | A |
| I-47 | B |
| I-48 | B |
| I-49-a | A |
| I-49-b | A |
| I-50 | B |
| I-51 | B |
| I-52 | C |
| I-53-a | C |
| I-53-b | C |
| I-54-a | C |
| I-54-b | C |
| I-55 | A |
| I-56 | B |
| I-57 | A |
| I-58-a | A |
| I-58-b | A |
| I-59 | A |
| I-60-a | C |
| I-60-b | C |
| I-61-a | A |
| I-61-b | A |
| I-62-a | A |
| I-62-b | A |
| I-63-a | A |
| I-63-b | A |
| I-64-a | A |
| I-64-b | A |
| I-65 | A |
| I-66 | A |
| I-67 | C |
| I-68 | A |
| I-69 | A |
| I-70-a | A |
| I-70-b | A |
| I-71 | C |
| I-72 | A |
| I-73 | A |
| I-74-a | A |
| I-74-b | C |
| I-75 | E |
| I-76 | A |
| I-77 | A |
| I-78 | B |
| I-79 | B |
| I-80 | B |
| I-81 | B |
| I-82-a | A |
| I-82-b | A |
| I-83-a | A |
| I-83-b | A |
| I-84-a | A |
| I-84-b | A |
| I-85 | A |
| I-86 | A |
| I-87-a | A |
| I-87-b | A |
| I-88-a | A |
| I-88-b | A |
| I-89-a | B |
| I-89-b | B |
| I-90-a | B |
| I-90-b | B |
| I-91 | C |
| I-92 | B |
| I-93 | B |
| I-94 | A |
| I-95-a | A |
| I-95-b | A |
| I-96-a | B |
| I-96-b | A |
| I-97-a | A |
| I-97-b | A |
| I-98 | B |
| I-99 | A |
| I-100-a | A |
| I-100-b | A |
| I-101-a | D |
| I-101-b | D |
| I-102-a | A |
| I-102-b | A |
| I-103-a | A |
| I-103-b | A |
| I-104-a | A |
| I-104-b | A |
| I-105-a | A |
| I-105-b | A |
| I-106-a | A |
| I-106-b | A |
| I-107-a | A |
| I-107-b | A |
| I-108-a | B |
| I-108-b | B |
| I-109-a | B |
| I-109-b | B |
| I-110-a | A |
| I-110-b | A |
| I-111-a | A |
| I-111-b | A |
| I-112 | B |
| I-113-a | C |
| I-113-b | D |
| I-114-a | A |
| I-114-b | A |
| I-115-a | B |
| I-115-b | C |
| I-116 | A |
| I-117-a | C |
| I-117-b | C |
| I-118-a | B |
| I-118-b | C |
| I-119 | D |
| I-120-a | C |
| I-120-b | A |
| I-121 | A |
| I-122-a | A |
| I-122-b | A |
| I-123-a | A |
| I-123-b | A |
| I-124 | A |
| I-125-a | A |
| I-125-b | A |
| I-126-a | A |
| I-126-b | A |
| I-127-a | C |
| I-127-b | C |
| I-128-a | B |
| I-128-b | A |
| I-129-a | A |
| I-129-b | A |
| I-130 | C |
| I-131-a | A |
| I-131-b | A |
| I-132-a | B |
| I-132-b | B |
| I-133-a | A |
| I-133-b | A |
| I-134-a | C |
| I-134-b | B |
| I-135-a | B |
| I-135-b | C |
| I-136-a | A |
| I-136-b | A |
| I-137 | B |
| I-138 | A |
| I-139 | A |
| I-140-a | A |
| I-140-b | A |
| I-142 | C |
| I-143 | B |
| I-144 | A |
| I-145 | A |
| I-146-a | A |
| I-146-b | A |
| I-147-a | A |
| I-147-b | A |
| I-148-a | A |
| I-148-b | A |
| I-149 | A |
| I-150 | A |
| I-151 | A |
| I-152 | A |
| I-153-a | A |
| I-153-b | B |
| I-154-a | A |
| I-154-b | A |
| I-155-a | B |
| I-155-b | B |
| I-156 | A |
| I-157 | A |
| I-158 | A |
| I-159-a | A |
| I-159-b | A |
| I-160-a | A |
| I-160-b | A |
| I-161-a | C |
| I-161-b | B |
| I-162-a | A |
| I-162-b | B |
| I-163-a | B |
| I-163-b | B |
| I-164-a | C |
| I-164-b | B |
| I-165-a | A |
| I-165-b | A |
| I-166 | A |
| I-166-a | C |
| I-166-b | E |
| I-167-a | C |
| I-167-b | C |
| I-168 | A |
| I-169-a | B |
| I-169-b | A |
| I-170 | A |
| I-171 | C |
| I-172-a | A |
| I-172-b | A |
| I-173 | A |
| I-174 | A |
| I-175 | A |
| I-176 | A |
| I-177 | A |
| I-178 | A |
| I-179 | B |
| I-180 | A |
| I-181 | A |
| I-182 | A |
| I-183-a | A |
| I-183-b | A |
| I-184-a | C |
| I-184-b | D |

### JAK Family Selectivity Assays

Provided compounds are evaluated for selectivity by comparing their JAK2 binding affinity (K_{d}) in the above JAK2 Binding Assay with their binding affinity (K_{d}) for one or more other kinases. Binding affinity for other kinases is determined as follows: Kinase-tagged T7 phage strains are prepared in an E. coli host derived from the BL21 strain. E. coli are grown to log-phase and infected with T7 phage and incubated with shaking at 32° C. until lysis. The lysates are centrifuged and filtered to remove cell debris. The remaining kinases are produced in HEK-293 cells and subsequently tagged with DNA for qPCR detection. Streptavidin-coated magnetic beads are treated with biotinylated small molecule ligands for 30 minutes at room temperature to generate affinity resins for kinase assays. The liganded beads are blocked with excess biotin and washed with blocking buffer (SeaBlock (Pierce), 1% BSA, 0.05% Tween 20, 1 mM DTT) to remove unbound ligand and to reduce non-specific binding. Binding reactions are assembled by combining kinases, liganded affinity beads, and test compounds in 1× binding buffer (20% SeaBlock, 0.17× PBS, 0.05% Tween 20, 6 mM DTT). Test compounds are prepared as 111× stocks in 100% DMSO. Kds are determined using an 11-point 3-fold compound dilution series with three DMSO control points. All compounds for K_{d} measurements are distributed by acoustic transfer (non-contact dispensing) in 100% DMSO. The compounds are then diluted directly into the assays such that the final concentration of DMSO is 0.9%. All reactions are performed in polypropylene 384-well plate. Each has a final volume of 0.02 ml. The assay plates are incubated at room temperature with shaking for 1 hour and the affinity beads are washed with wash buffer (1× PBS, 0.05% Tween 20). The beads are then re-suspended in elution buffer (1× PBS, 0.05% Tween 20, 0.5 μM non-biotinylated affinity ligand) and incubated at room temperature with shaking for 30 minutes. The kinase concentration in the eluates is measured by qPCR. Compounds that exhibit a better binding affinity for JAK2 compared to one or more other kinases are considered to be JAK2-selective compounds. In some embodiments, provided compounds may be JAK2-selective over one or more of the following kinases: JAK1, JAK3, and Tyk2.

### SET2-pSTAT5 Cellular Assay

This assay measures inhibition of JAK2-mediated pSTAT5 signaling in constitutively active essential thrombocytopenia cells carrying the V617F mutation. Cells are harvested from a flask into cell culture medium, and the number of cells is counted. The cells are diluted with culture medium and 100 μL of cell suspension (50000/well) is added into each well of a 96-well cell culture plate. A solution of test compound is added to the assay plate. The plates are covered with a lid and placed in a 37° C. 5% CO₂ incubator for 4 hours. After 4 hours, the cells are spun, and the cell pellets are re-suspended with 100 μL cold PBS. Then, the cells are spun again at 4° C. and 4000 rpm for 5 min. PBS is aspirated, and 25 μL lysis buffer (with protease and phosphatase inhibitor cocktail) is added to each cell pellet. The cell lysate is shaken at 4° C. for 20 min to fully lyse the cells. The cell lysate is spun at 4° C. and 4000 rpm for 15 min, and then the supernatant is transferred into a new plate and stored at −80° C. Meso-scale discovery (MSD) is used to analyze plates as follows: a standard MSD plate is coated with capture antibody in PBS (40 μL/well) and is incubated at 4° C. overnight with shaking. The MSD plate is washed three times with 150 μL/well of 1× MSD Wash Buffer (Tris-buffered saline with 0.1% Tween® 20 detergent, TBST). The MSD plates are then blocked with 150 μL of blocking buffer (5% BSA in TBST) and shaken for 1 h at room temperature and 600 rpm. The MSD plate is washed three times with 150 μL/well of 1× MSD Wash Buffer (TBST). Sample lysates are then added to MSD plates (25 μL/well) and shaken for 1 h at room temperature and 600 rpm. The MSD plate is washed three times with 150 μL/well of 1× MSD Wash Buffer (TBST). Detection antibody (prepared in Antibody Detection buffer, 1% BSA in 1×TBST) is then added to the MSD plates, and they are shaken for 1 h at room temperature and 600 rpm. The MSD plate is washed three times with 150 μL/well of 1× MSD Wash Buffer (TBST). A secondary detection antibody (prepared in Antibody Detection buffer, 1% BSA in 1×TBST) is then added to the MSD plates, and they are shaken for 1 h at room temperature and 600 rpm. The MSD plate is washed three times with 150 μL/well of 1× MSD Wash Buffer (TBST). MSD reading buffer (1×) is added to the plates (150 μL/well), and they are diluted from 4× with water. The plates are imaged using an MSD imaging instrument according to the manufacturer's instructions.

### Caco2 Permeability Assay

Preparation of Caco-2 Cells: 50 μL and 25 mL of cell culture medium are added to each well of a Transwell® insert and reservoir, respectively. Then, the HTS Transwell® plates are incubated at 37° C., 5% CO₂ for 1 hour before cell seeding. Caco-2 cell cells are diluted to 6.86×105 cells/mL with culture medium, and 50 μL of cell suspension are dispensed into the filter well of the 96-well HTS Transwell® plate. Cells are cultivated for 14-18 days in a cell culture incubator at 37° C., 5% CO₂, 95% relative humidity. Cell culture medium is replaced every other day, beginning no later than 24 hours after initial plating.

Preparation of Stock Solutions: 10 mM stock solutions of test compounds are prepared in DMSO. The stock solutions of positive controls are prepared in DMSO at the concentration of 10 mM. Digoxin and propranolol are used as control compounds in this assay.

Assessment of Cell Monolayer Integrity: Medium is removed from the reservoir and each Transwell® insert and is replaced with prewarmed fresh cuture medium. Transepithelial electrical resistance (TEER) across the monolayer is measured using Millicell Epithelial Volt-Ohm measuring system (Millipore, USA). The Plate is returned to the incubator once the measurement is done. The TEER value isss calucated according to the following equation: TEER measurement (ohms)×Area of membrane (cm²)=TEER value (ohm·cm²). A TEER value greater than 230 ohm·cm² indicates a well-qualified Caco-2 monolayer.

Assay Procedure: The Caco-2 plate is removed from the incubator and washed twice with pre-warmed HBSS (10 mM HEPES, pH 7.4), and then incubated at 37° C. for 30 minutes. The stock solutions of control compounds are diluted in DMSO to get 1 mM solutions and then diluted with HBSS (10 mM HEPES, pH 7.4) to get 5 μM working solutions. The stock solutions of the test compounds are diluted in DMSO to get 1 mM solutions and then diluted with HBSS (10 mM HEPES and 4% BSA, pH 7.4) to get 5 μM working solutions. The final concentration of DMSO in the incubation system is 0.5%. To determine the rate of drug transport in the apical to basolateral direction. 75 μL of 5 μM working solutions of test compounds are added to the Transwell® insert (apical compartment) and the wells in the receiver plate (basolateral compartment) are filled with 235 μL of HBSS (10 mM HEPES and 4% BSA, pH 7.4). To determine the rate of drug transport in the basolateral to apical direction, 235 μL of 5 μM working solutions of test compounds are added to the receiver plate wells (basolateral compartment) and then the Transwell® inserts (apical compartment) are filled with 75 μL of HBSS (10 mM HEPES and 4% BSA, pH 7.4). Time 0 samples are prepared by transferring 50 μL of 5 μM working solution to wells of the 96-deepwell plate, followed by the addition of 200 μL cold methanol containing appropriate internal standards (IS). The plates are incuabted at 37° C. for 2 hours. At the end of the incubation, 50 μL samples from donor sides (apical compartment for Ap→Bl flux, and basolateral compartment for Bl→Ap) and receiver sides (basolateral compartment for Ap→Bl flux, and apical compartment for Bl→Ap) are transferred to wells of a new 96-well plate, followed by the addition of 4 volume of cold acetonitrile or methanol containing appropriate internal standards (IS). Samples are vortexed for 5 minutes and then centrifuged at 3,220 g for 40 minutes. An aliquot of 100 μL of the supernatant is mixed with an appropriate volume of ultra-pure water before LC-MS/MS analysis. To determine the Lucifer Yellow leakage after 2 hour transport period, stock solution of Lucifer yellow is prepared in ultra-pure water and diluted with HBSS (10 mM HEPES, pH 7.4) to reach the final concentration of 100 μM. 100 μL of the Lucifer yellow solution is added to each Transwell® insert (apical compartment), followed by filling the wells in the receiver plate (basolateral compartment) with 300 μL of HBSS (10 mM HEPES, pH 7.4). The plates are incubated at 37° C. for 30 minutes. 80 L samples are removed directly from the apical and basolateral wells (using the basolateral access holes) and transferred to wells of new 96 wells plates. The Lucifer Yellow fluorescence (to monitor monolayer integrity) signal is measured in a fluorescence plate reader at 485 nM excitation and 530 nM emission.

### Cytotoxicity Assay

HEK293T cells are harvested from flask into cell culture medium, and then the cells are counted. The cells are diluted with culture medium to the desired density, and 40 μL of cell suspension is added into each well of a 384-well cell culture plate. The plates are covered with a lid and spun at room temperature at 1,000 RPM for 1 minute and then transferred into 37° C. 5% CO₂ incubator overnight. Test compounds are dissolved at 10 mM DMSO stock solution. 45 μL of stock solution is then transferred to a 384 PP-plate. A 3-fold, 10-point dilution is performed via transferring 15 μL compound into 30 μL DMSO by using TECAN (EVO200) liquid handler. The plates are spun at room temperature at 1,000 RPM for 1 minute and shaken on a plate shaker for 2 minutes. 40 nL of diluted compound is transferred from compound source plate into the cell plate by using liquid handler Echo550. After compound treatment for 48 hours, CTG detection is performed for compound treatment plates: the plates are removed from incubators and equilibrated at room temperature for 15 minutes. 30 μL of CellTiter-Glo reagent is added into each well to be detected. The plates are then placed at room temperature for 30 min followed by reading on EnVision. Inhibition activity is calculated with the following formula: % Inhibition=100×(LumHC−LumSample)/(LumHC−LumLC), wherein HC is reading obtained from cells treated with 0.1% DMSO only and LC is reading from cells treated with 10 L staurosporine. IC₅₀ values are calculated using XLFit (equation 201).

### Hepatocyte Stability Assay

10 mM stock solutions of test compound and positive control are prepared in DMSO. Stock solutions are diluted to 100 μM by combining 198 μL of 50% acetonitrile/50% water and 2 μL of 10 mM stock solution. Verapamil is used as positive control in the assay. Vials of cryopreserved hepatocytes are thawed in a 37° C. water bath with gently shaking. The contents are poured into the 50 mL thawing medium conical tube. Vials are centrifuged at 100 g for 10 minutes at room temperature. Thawing medium is aspirated and hepatocytes are re-suspended with serum-free incubation medium to yield ˜1.5×106 cells/mL. Cell viability and density are counted using a Trypan Blue exclusion, and then cells are diluted with serum-free incubation medium to a working cell density of 0.5×106 viable cells/mL. A portion of the hepatocytes at 0.5×106 viable cells/mL are boiled for 5 min prior to adding to the plate as negative control to eliminate the enzymatic activity so that little or no substrate turnover should be observed. Aliquots of 198 μL hepatocytes are dispensed into each well of a 96-well non-coated plate. The plate is placed in the incubator for approximately 10 minutes. Aliquots of 2 μL of the 100 μM test compound and 2 μL positive control are added into respective wells of a non-coated 96-well plate to start the reaction. The final concentration of test compound is 1 PM. This assay is performed in duplicate. The plate is incubated in the incubator for the designed time points. 25 L of contents are transferred and mixed with 6 volumes (150 μL) of cold acetonitrile with internal standard (100 nM alprazolam, 200 nM labetalol, 200 nM caffeine and 200 nM diclofenac) to terminate the reaction at time points of 0, 15, 30, 60, 90 and 120 minutes. Samples are centrifuged for 25 minutes at 3,220 g and aliquots of 150 μL of the supernatants are used for LC-MS/MS analysis.

### Kinetic Solubility Assay

Stock solutions of test compounds are prepared in DMSO at the concentration of 10 mM, and a stock solution of control compound is prepared in DMSO at the concentration of 30 mM. Diclofenac is used as positive control in the assay. 30 μL stock solution of each compound is placed into their a 96-well rack, followed by adding 970 μL of PBS at pH 4.0 and pH 7.4 into each vial of the cap-less solubility sample plate. This study is performed in duplicate. One stir stick is added to each vial and then vials are sealed using a molded PTDE/SIL 96-Well Plate Cover. The solubility sample plate is transferred to the Thermomixer comfort plate shaker and incubated at RT for 2 hours with shaking at 1100 rpm. After 2 hours incubation, stir sticks are removed using a big magnet and all samples from the solubility sample plate are transferred into the filter plate. All the samples are filtered by vacuum manifold. The filtered samples are diluted with methanol. Samples are analyzed by LC-MS/MS and quantified against a standard of known concentration in DMSO using LC coupled with Mass spectral peak identification and quantitation. The solubility values of the test compounds are calculated as follows, wherein INJ VOL is injection volume, DF is dilution factor, and STD is standard:

$$[ Sample ] = \frac{{AREA}_{Sample} \times INJ {VOL}_{Std} \times {DF}_{Sample} \times [ STD ]}{{AREA}_{Std} \times INJ {VOL}_{Sample}}$$

### Plasma Protein Binding Assay

Working solutions of test compounds and control compound are prepared in DMSO at the concentration of 200 μM, and then the working solutions are spiked into plasma. The final concentration of compound is 1 μM. The final concentration of DMSO is 0.5%. Ketoconazole is used as positive control in the assay. Dialysis membranes are soaked in ultrapure water for 60 minutes to separate strips, then in 20% ethanol for 20 minutes, finally in dialysis buffer for 20 minutes. The dialysis set up is assembled according to the manufacturer's instruction. Each Cell is with 150 μL of plasma sample and dialyzed against equal volume of dialysis buffer (PBS). The assay is performed in duplicate. The dialysis plate is sealed and incubated in an incubator at 37° C. with 5% CO₂ at 100 rpm for 6 hours. At the end of incubation, 50 μL of samples from both buffer and plasma chambers are transferred to wells of a 96-well plate. 50 μL of plasma is added to each buffer samples and an equal volume of PBS is supplemented to the collected plasma sample. 400 μL of precipitation buffer acetonitrile containing internal standards (IS, 100 nM alprazolam, 200 nM labetalol, 200 nM imipramine and 2 μM ketoplofen) is added to precipitate protein and release compounds. Samples are vortexed for 2 minutes and centrifuged for 30 minutes at 3,220 g. Aliquot of 50 μL of the supernatant is diluted by 150 μL acetonitrile containing internal standards: ultra-pure H₂O=1:1, and the mixture is used for LC-MS/MS analysis.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 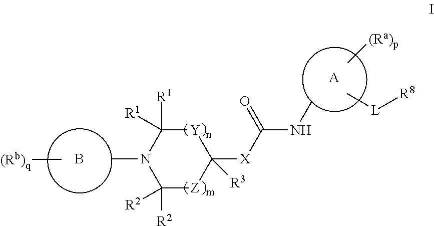
or a pharmaceutically acceptable salt thereof, wherein:
X is —C(R⁶)₂—, —N(R⁷)—, or —O—;
each Y is —C(R⁴)₂—;
each Z is —C(R⁵)₂—;
n is 0, 1, or 2;
m is 0, 1, or 2,
provided that at least one of n or m is 1 or 2;
each R¹, R², R³, R⁴, and R⁵ is independently hydrogen, halogen, —CN, —OR, or optionally substituted C₁₋₆ aliphatic, and/or
two R¹ groups and/or two R² groups and/or two R⁴ groups and/or two R⁵ groups, together with the atom(s) to which they are attached, combine to form a 3- to 8-membered saturated or partially unsaturated ring, and/or
two R¹ groups and/or two R² groups and/or two R⁴ groups and/or two R⁵ groups, together with the atom to which they are attached, combine to form an oxo, and/or
a R¹ and R² group and/or a R¹ and R³ group and/or a R¹ and R⁴ group and/or a R¹ and R⁵ group and/or a R² and R³ group and/or a R² and R⁴ group and/or a R² and R⁵ group and/or a R³ and R⁴ group and/or a R³ and R⁵ group and/or a R³ and R⁶ group and/or a R³ and R⁷ group and/or a R⁴ and R⁵ group and/or a R⁴ and R⁶ group and/or a R⁴ and R⁷ group and/or a R⁵ and R⁶ group and/or a R⁵ and R⁷ group, together with the atoms to which they are attached, combine to form an optionally substituted 3- to 8-membered saturated or partially unsaturated ring;
each R⁶ is independently hydrogen or optionally substituted C₁₋₆ aliphatic, or
two R⁶ groups, together with the atom to which they are attached, combine to form a 3- to 8-membered saturated or partially unsaturated ring;
R⁷ is hydrogen or optionally substituted C₁₋₆ aliphatic;
Ring A is an optionally substituted group selected from phenyl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 7- to 10-membered saturated or partially unsaturated bicyclic heterocyclyl having 1-4 heteroatoms independently selected from nitrogen, oxygen, and sulfur;
L is a covalent bond or a bivalent C₁₋₃ straight or branched hydrocarbon chain;
R⁸ is hydrogen, halogen, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, optionally substituted 5- to 6-membered monocyclic heteroaryl having 1-4 heteroatoms independently selected from nitrogen, oxygen, and sulfur, or optionally substituted 7- to 10-membered saturated or partially unsaturated bicyclic heteroaryl having 1-4 heteroatoms independently selected from nitrogen, oxygen, and sulfur;
each R^{a} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur; 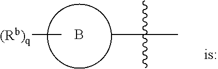
(i) 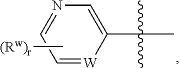
wherein:
W is CH, CR^{w}, or N:
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
two R^{w} groups, together with the atoms to which they are attached, combine to form an 5- to 6-membered partially unsaturated or aromatic ring substituted with q R^{b} groups; and
r is 0, 1, 2, or 3; or
(ii) 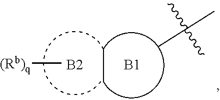
wherein:
Ring B1 is a 5- or 6-membered monocyclic heteroaryl having 1-3 heteroatoms independently selected from nitrogen, oxygen, and sulfur, wherein Ring B1 is fused to Ring B2; and
Ring B2 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur:
each R^{b} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur;
p is 0, 1, 2, 3, 4, or 5, as valency permits;
q is 0, 1, 2, 3, 4, or 5, as valency permits;
each R is independently hydrogen or an optionally substituted group selected from C₁₋₆ aliphatic, C₃₋₇ cycloaliphatic, 3- to 7-membered saturated or partially unsaturated monocyclic heterocyclyl having 1-3 heteroatoms independently selected from nitrogen, oxygen, and sulfur, phenyl, and 5- to 6-membered monocyclic heteroaryl having 1-4 heteroatoms independently selected from nitrogen, oxygen, and sulfur, or
two R, when attached to the same nitrogen atom, are taken together to form an optionally substituted 3- to 7-membered saturated or partially unsaturated ring having 0-1 additional heteroatoms independently selected from nitrogen, oxygen, and sulfur; and
each R′ is independently an optionally substituted group selected from C₁₋₆ aliphatic and C₃₋₇ cycloaliphatic, and
wherein the compound is not:

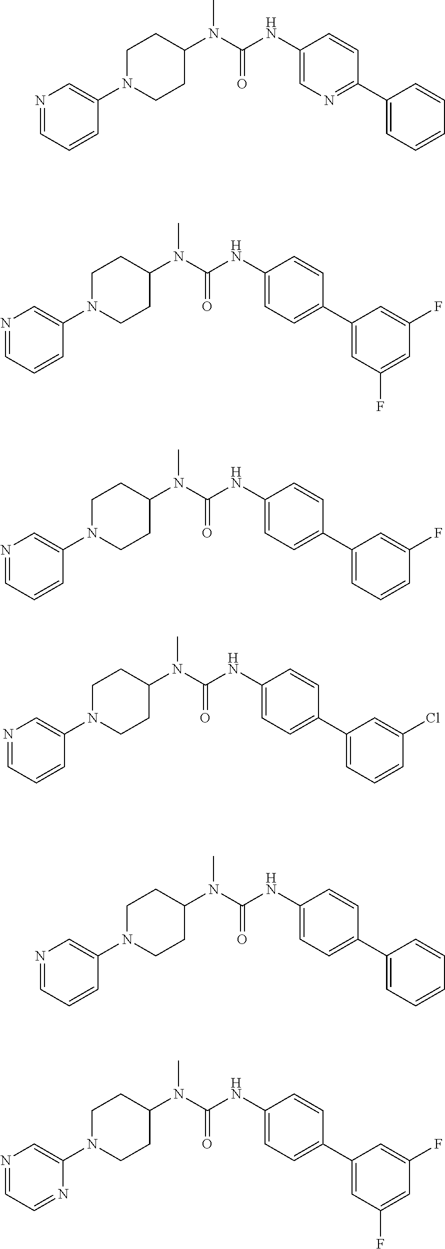 [SMILES: CN(C(=O)Nc1ccc(-c2ccccc2)nc1)C1CCN(c2cccnc2)CC1 ; CN(C(=O)Nc1ccc(-c2cc(F)cc(F)c2)cc1)C1CCN(c2cccnc2)CC1 ; CN(C(=O)Nc1ccc(-c2cccc(F)c2)cc1)C1CCN(c2cccnc2)CC1 ; CN(C(=O)Nc1ccc(-c2cccc(Cl)c2)cc1)C1CCN(c2cccnc2)CC1 ; CN(C(=O)Nc1ccc(-c2ccccc2)cc1)C1CCN(c2cccnc2)CC1 ; CN(C(=O)Nc1ccc(-c2cc(F)cc(F)c2)cc1)C1CCN(c2cnccn2)CC1]

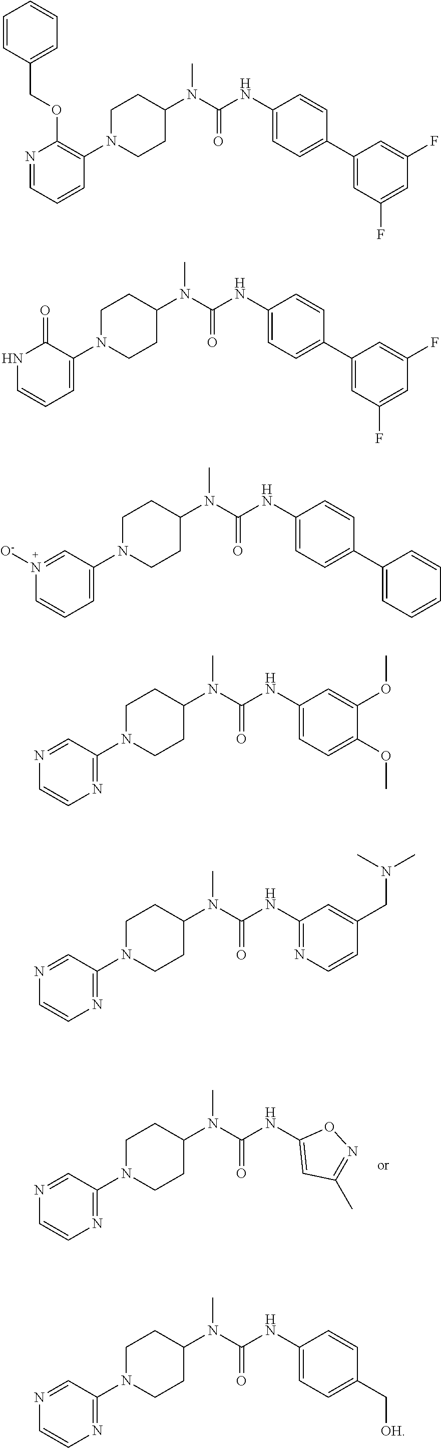

2-3. (canceled)

4. The compound of claim 1, wherein the moiety 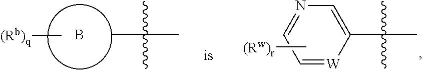
wherein:
W is CH, CR^{w}, or N;
each R^{w} is independently halogen, —CN, —OR, —O(CH₂)₁₋₄R, —SR, —N(R)₂, —NO₂, —C(O)R′, —C(O)OR, —C(O)N(R)₂, —OC(O)R′, —OC(O)N(R)₂, —OC(O)OR, —OSO₂R, —OSO₂N(R)₂, —N(R)C(O)R′, —N(R)C(O)OR, —N(R)C(O)N(R)₂, —N(R)SO₂R′, —SO₂R′, —SO₂N(R)₂, —SO₃R′, optionally substituted C₁₋₆ aliphatic, optionally substituted C₃₋₆ cycloaliphatic, optionally substituted 3- to 6-membered saturated or partially unsaturated monocyclic heterocyclyl having 1-3 heteroatoms independently selected from nitrogen, oxygen, and sulfur, optionally substituted phenyl, or optionally substituted 5- to 6-membered monocyclic heteroaryl having 1-4 heteroatoms independently selected from nitrogen, oxygen, and sulfur, or
two R^{w} groups, together with the atoms to which they are attached, combine to form an 5- to 6-membered partially unsaturated or aromatic ring substituted with q R^{b} groups; and
r is 0, 1, 2, or 3.

5. The compound of claim 4, wherein W is CH.

6. The compound of claim 4, wherein W is N.

7-8. (canceled)

9. The compound of claim 4, wherein r is 2.

10. The compound of claim 9, wherein two R^{w} groups, together with the atoms to which they are attached, combine to form a 5- to 6-membered partially unsaturated or aromatic ring substituted with q R^{b} groups.

11. The compound of claim 10, wherein each R^{b} is independently halogen, —CN, —OR, —N(R)₂, —N(R)C(O)R′, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.

12. The compound of claim 1, wherein the moiety 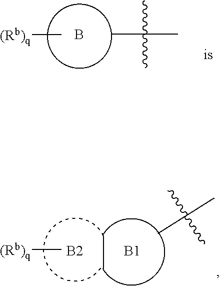
wherein:
Ring B1 is a 5- to 6-membered monocyclic heteroaryl having 1-3 heteroatoms independently selected from nitrogen, oxygen, and sulfur,
wherein Ring B1 is fused to Ring B2; and
Ring B2 is phenyl, 5- to 6-membered monocyclic heteroaryl having 1-3 heteroatoms independently selected from nitrogen, oxygen, and sulfur, C₅₋₆ cycloaliphatic, or 5- to 6-membered saturated or partially unsaturated monocyclic heterocyclyl having 1-3 heteroatoms independently selected from nitrogen, oxygen, and sulfur.

13-14. (canceled)

15. The compound of claim 12, wherein Ring B1 is a 6-membered monocyclic heteroaryl having 1-3 heteroatoms independently selected from nitrogen, oxygen, and sulfur.

16. (canceled)

17. The compound of claim 12, wherein Ring B2 is a 5- to 6-membered monocyclic heteroaryl having 1-3 heteroatoms independently selected from nitrogen, oxygen, and sulfur.

18. The compound of claim 12, wherein each R^{b} is independently halogen, —CN, —OR, —N(R)₂, —C(O)N(R)₂, —N(R)C(O)R′, —N(R)C(O)N(R)₂, optionally substituted C₁₋₆ aliphatic, or optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.

19-22. (canceled)

23. The compound of claim 1, wherein X is —N(R⁷)—.

24. (canceled)

25. The compound of claim 23, wherein R⁷ is C₁₋₆ alkyl.

26. (canceled)

27. The compound of claim 1, wherein n is 1.

28. The compound of claim 1, wherein m is 1.

29. (canceled)

30. The compound of claim 1, wherein each R¹, R², R³, and R⁴ is hydrogen, and each R⁵ is independently hydrogen, halogen, —CN, —OR, or optionally substituted C₁₋₆ aliphatic.

31-34. (canceled)

35. The compound of claim 1, wherein one of the following occurs:
(i) a R¹ and R⁵ group, together with the atoms to which they are attached, combine to form a 3- to 8-membered saturated or partially unsaturated ring;
(ii) a R³ and R⁷ group, together with the atoms to which they are attached, combine to form an optionally substituted 3- to 8-membered saturated or partially unsaturated ring;
(iii) a R⁴ and R⁵ group, together with the atoms to which they are attached, combine to form a 3- to 8-membered saturated or partially unsaturated ring; or
(iv) a R⁴ and R⁷ group, together with the atoms to which they are attached, combine to form a 3- to 8-membered saturated or partially unsaturated ring.

36. The compound of claim 1, wherein Ring A is phenyl or 5- to 6-membered monocyclic heteroaryl having 1-4 heteroatoms independently selected from nitrogen, oxygen, and sulfur.

37. (canceled)

38. The compound of claim 36, wherein Ring A is 5- to 6-membered monocyclic heteroaryl having 1-4 heteroatoms independently selected from nitrogen, oxygen, and sulfur.

39. The compound of claim 36, wherein L is a covalent bond.

40-41. (canceled)

42. The compound of claim 1, wherein R⁸ is optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.

43. The compound of claim 1, wherein each R^{a} is independently halogen, —CN, —OR, —O(CH₂)₁₋₄R, optionally substituted C₁₋₆ aliphatic, optionally substituted C₃₋₆ cycloaliphatic, or optionally substituted 3- to 6-membered saturated or partially unsaturated monocyclic heterocyclyl having 1-3 heteroatoms independently selected from nitrogen, oxygen, and sulfur.

44. The compound of claim 43, wherein each R^{a} is optionally substituted C₁₋₆ aliphatic.

45. The compound of claim 44, wherein each R^{a} is C₁₋₆ alkyl optionally substituted with one or more halogen.

46. The compound of claim 1, wherein p is 0 or 1.

47. The compound of claim 1, wherein a moiety 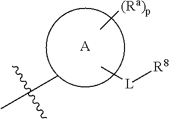
is selected from:

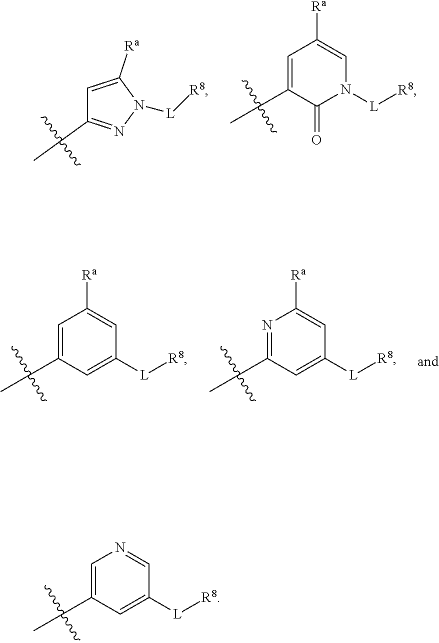

48-51. (canceled)

52. The compound of claim 1, wherein the compound is of Formula IB: 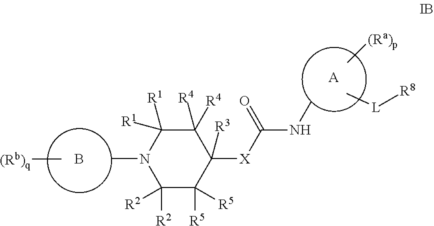
or a pharmaceutically acceptable salt thereof.

53-56. (canceled)

57. The compound of claim 1, wherein the compound is of Formula IV: 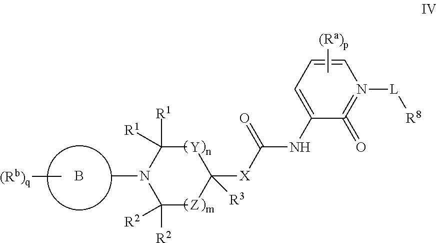
or a pharmaceutically acceptable salt thereof.

58. The compound of claim 1, wherein the compound is of Formula V: 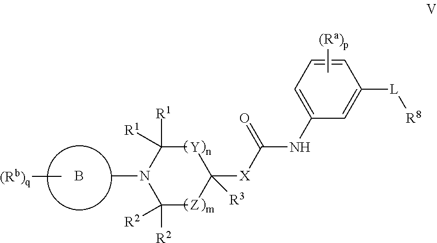
or a pharmaceutically acceptable salt thereof.

59-66. (canceled)

67. A compound selected from: 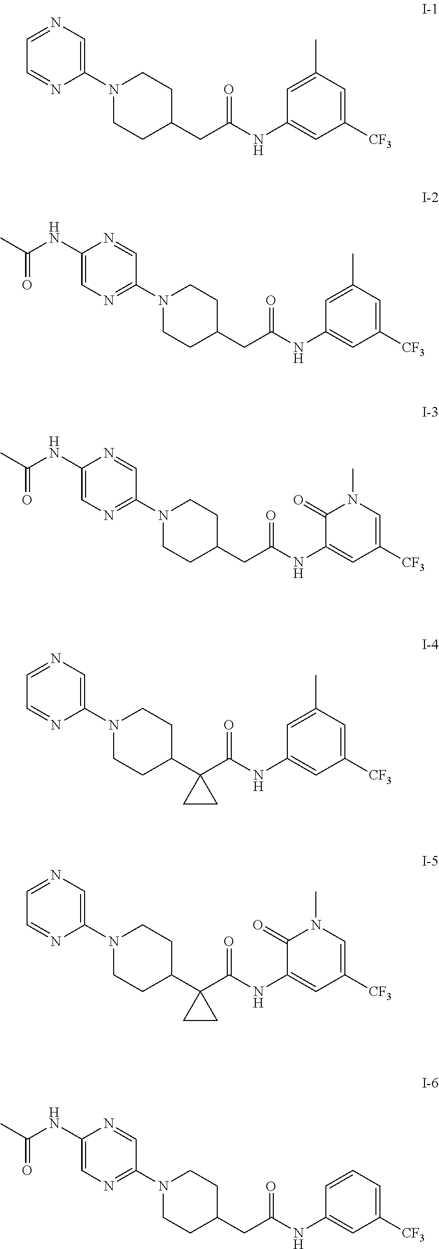 [SMILES: Cc1cc(NC(=O)CC2CCN(c3cnccn3)CC2)cc(C(F)(F)F)c1 ; CC(=O)Nc1cnc(N2CCC(CC(=O)Nc3cc(C)cc(C(F)(F)F)c3)CC2)cn1 ; CC(=O)Nc1cnc(N2CCC(CC(=O)Nc3cc(C(F)(F)F)cn(C)c3=O)CC2)cn1 ; Cc1cc(NC(=O)C2(C3CCN(c4cnccn4)CC3)CC2)cc(C(F)(F)F)c1 ; Cn1cc(C(F)(F)F)cc(NC(=O)C2(C3CCN(c4cnccn4)CC3)CC2)c1=O ; CC(=O)Nc1cnc(N2CCC(CC(=O)Nc3cccc(C(F)(F)F)c3)CC2)cn1] 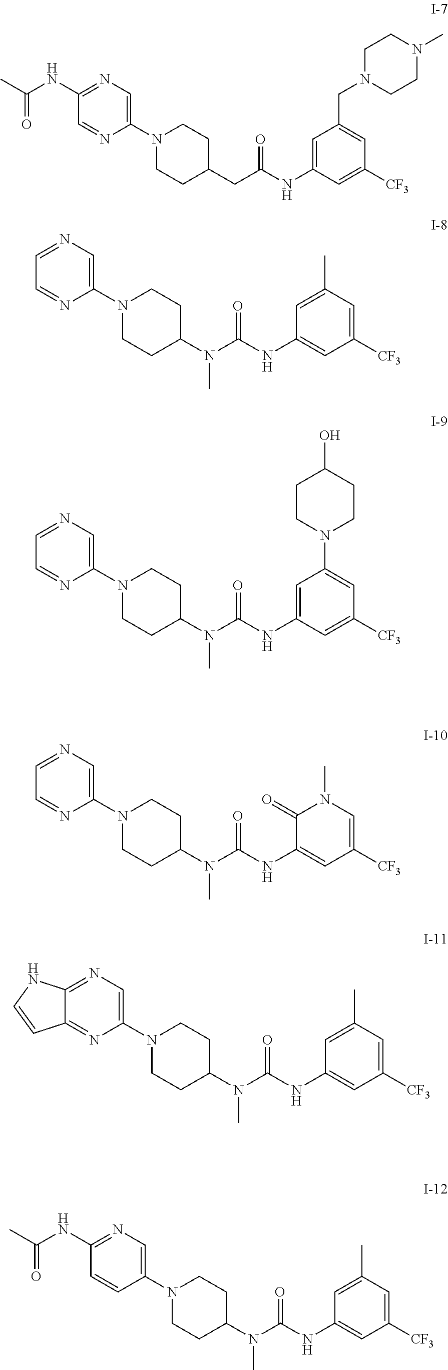 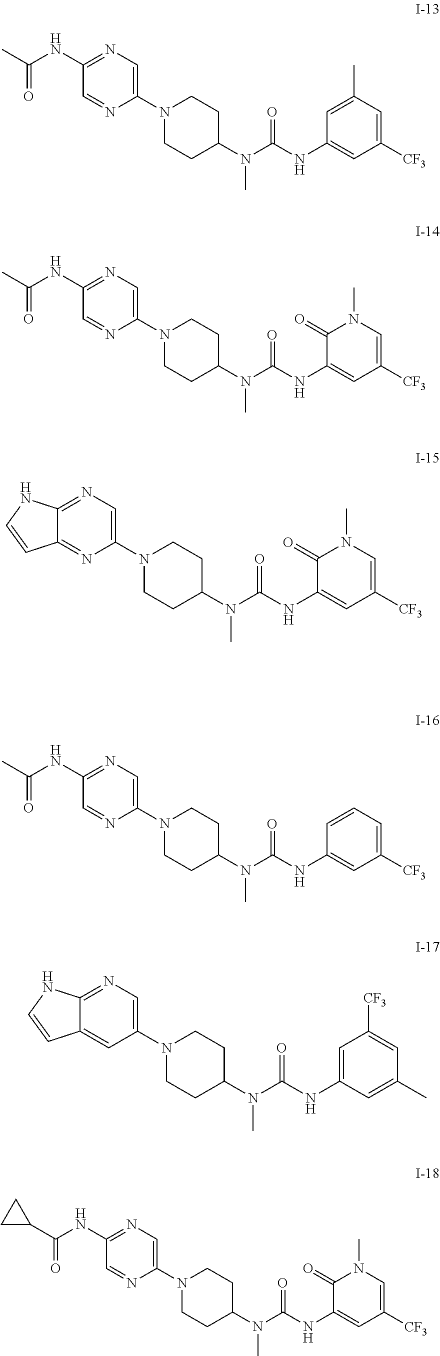 [SMILES: CC(=O)Nc1cnc(N2CCC(N(C)C(=O)Nc3cc(C)cc(C(F)(F)F)c3)CC2)cn1 ; CC(=O)Nc1cnc(N2CCC(N(C)C(=O)Nc3cc(C(F)(F)F)cn(C)c3=O)CC2)cn1 ; CN(C(=O)Nc1cc(C(F)(F)F)cn(C)c1=O)C1CCN(c2cnc3[nH]ccc3n2)CC1 ; CC(=O)Nc1cnc(N2CCC(N(C)C(=O)Nc3cccc(C(F)(F)F)c3)CC2)cn1 ; Cc1cc(NC(=O)N(C)C2CCN(c3cnc4[nH]ccc4c3)CC2)cc(C(F)(F)F)c1 ; CN(C(=O)Nc1cc(C(F)(F)F)cn(C)c1=O)C1CCN(c2cnc(NC(=O)C3CC3)cn2)CC1] 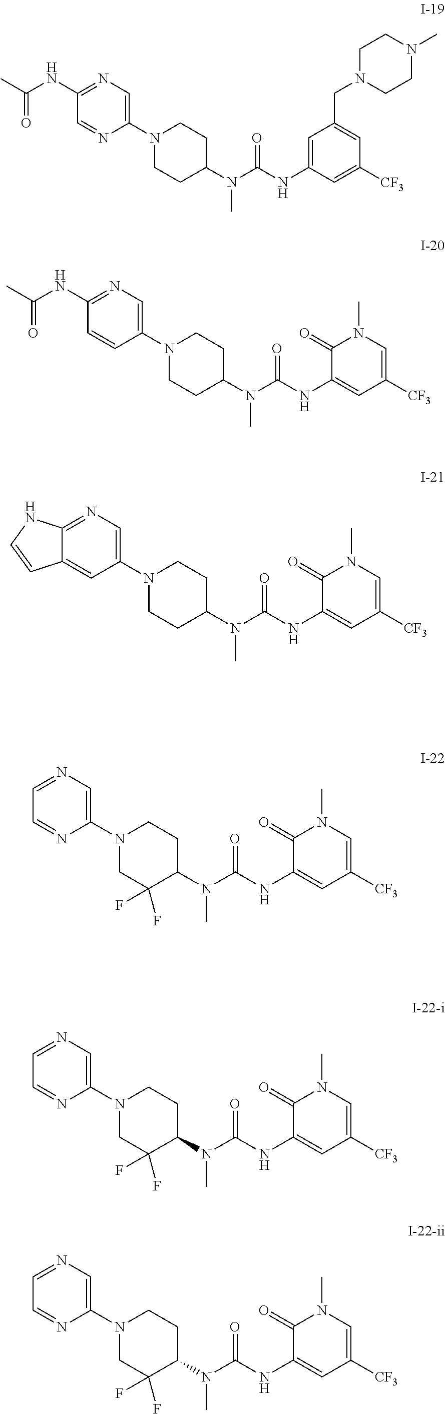 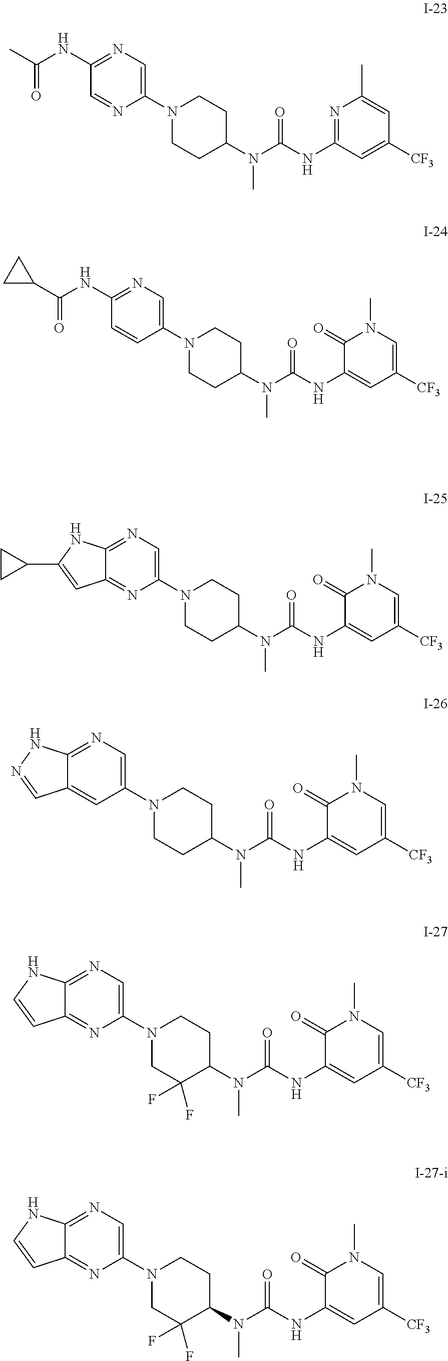 [SMILES: CC(=O)Nc1cnc(N2CCC(N(C)C(=O)Nc3cc(C(F)(F)F)cc(C)n3)CC2)cn1 ; CN(C(=O)Nc1cc(C(F)(F)F)cn(C)c1=O)C1CCN(c2ccc(NC(=O)C3CC3)nc2)CC1 ; CN(C(=O)Nc1cc(C(F)(F)F)cn(C)c1=O)C1CCN(c2cnc3[nH]c(C4CC4)cc3n2)CC1 ; CN(C(=O)Nc1cc(C(F)(F)F)cn(C)c1=O)C1CCN(c2cnc3[nH]ncc3c2)CC1 ; CN(C(=O)Nc1cc(C(F)(F)F)cn(C)c1=O)C1CCN(c2cnc3[nH]ccc3n2)CC1(F)F ; CN(C(=O)Nc1cc(C(F)(F)F)cn(C)c1=O)[C@@H]1CCN(c2cnc3[nH]ccc3n2)CC1(F)F] 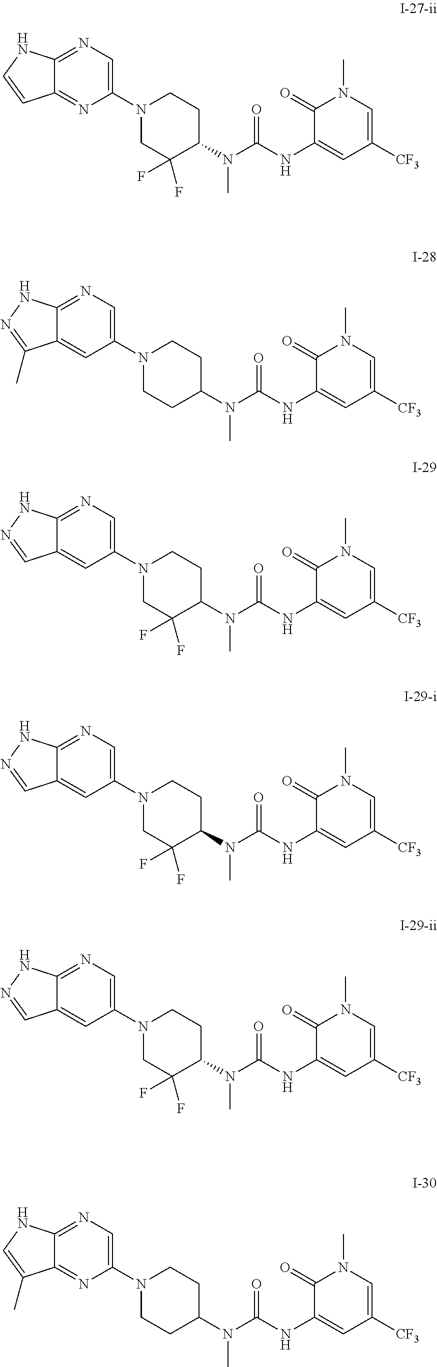 [SMILES: CN(C(=O)Nc1cc(C(F)(F)F)cn(C)c1=O)[C@H]1CCN(c2cnc3[nH]ccc3n2)CC1(F)F ; Cc1n[nH]c2ncc(N3CCC(N(C)C(=O)Nc4cc(C(F)(F)F)cn(C)c4=O)CC3)cc12 ; CN(C(=O)Nc1cc(C(F)(F)F)cn(C)c1=O)C1CCN(c2cnc3[nH]ncc3c2)CC1(F)F ; CN(C(=O)Nc1cc(C(F)(F)F)cn(C)c1=O)[C@@H]1CCN(c2cnc3[nH]ncc3c2)CC1(F)F ; CN(C(=O)Nc1cc(C(F)(F)F)cn(C)c1=O)[C@H]1CCN(c2cnc3[nH]ncc3c2)CC1(F)F ; Cc1c[nH]c2ncc(N3CCC(N(C)C(=O)Nc4cc(C(F)(F)F)cn(C)c4=O)CC3)nc12] 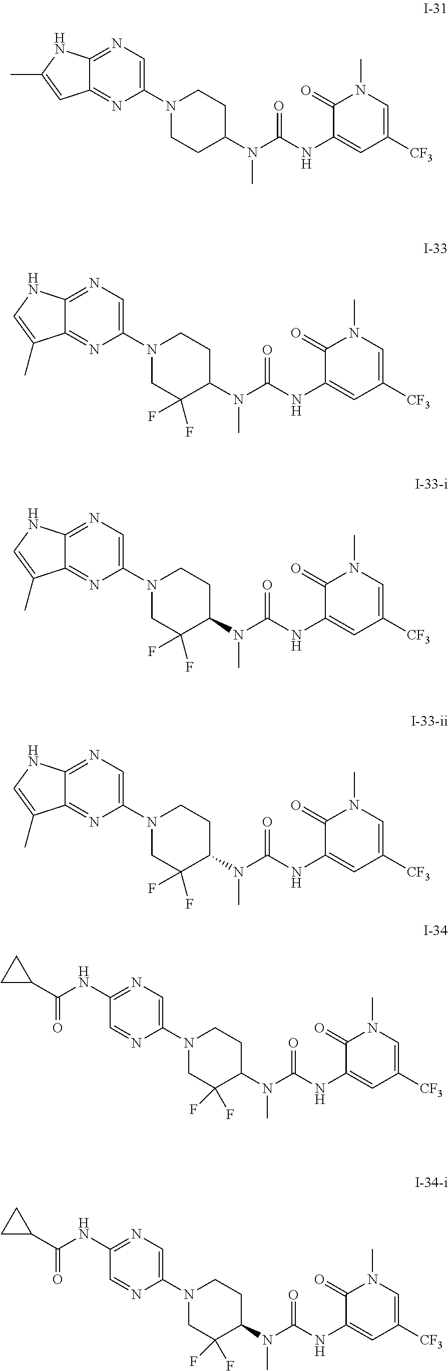 [SMILES: Cc1cc2nc(N3CCC(N(C)C(=O)Nc4cc(C(F)(F)F)cn(C)c4=O)CC3)cnc2[nH]1 ; Cc1c[nH]c2ncc(N3CCC(N(C)C(=O)Nc4cc(C(F)(F)F)cn(C)c4=O)C(F)(F)C3)nc12 ; Cc1c[nH]c2ncc(N3CC[C@@H](N(C)C(=O)Nc4cc(C(F)(F)F)cn(C)c4=O)C(F)(F)C3)nc12 ; Cc1c[nH]c2ncc(N3CC[C@H](N(C)C(=O)Nc4cc(C(F)(F)F)cn(C)c4=O)C(F)(F)C3)nc12 ; CN(C(=O)Nc1cc(C(F)(F)F)cn(C)c1=O)C1CCN(c2cnc(NC(=O)C3CC3)cn2)CC1(F)F ; CN(C(=O)Nc1cc(C(F)(F)F)cn(C)c1=O)[C@@H]1CCN(c2cnc(NC(=O)C3CC3)cn2)CC1(F)F] 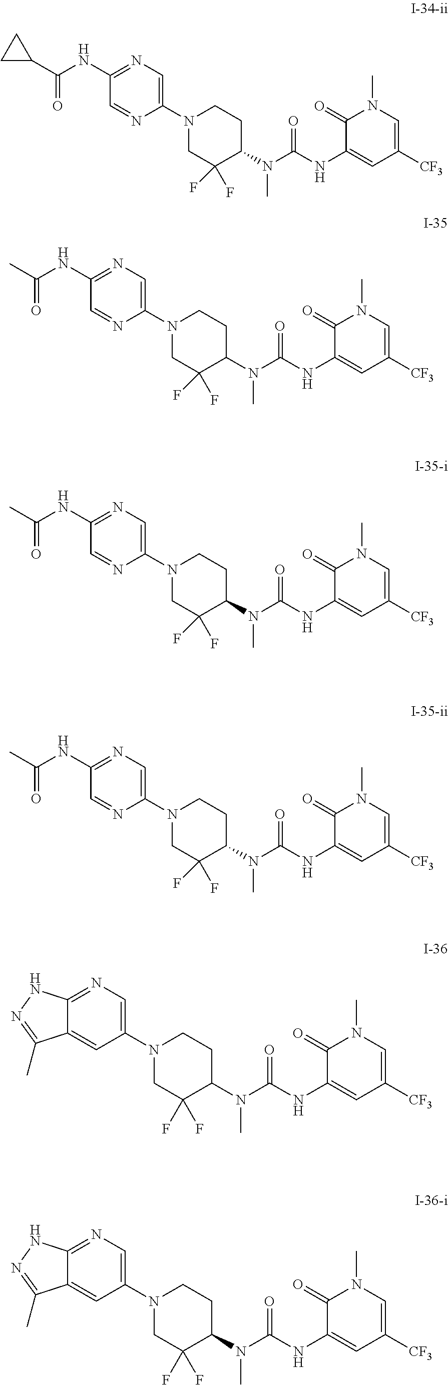 [SMILES: CN(C(=O)Nc1cc(C(F)(F)F)cn(C)c1=O)[C@H]1CCN(c2cnc(NC(=O)C3CC3)cn2)CC1(F)F ; CC(=O)Nc1cnc(N2CCC(N(C)C(=O)Nc3cc(C(F)(F)F)cn(C)c3=O)C(F)(F)C2)cn1 ; CC(=O)Nc1cnc(N2CC[C@@H](N(C)C(=O)Nc3cc(C(F)(F)F)cn(C)c3=O)C(F)(F)C2)cn1 ; CC(=O)Nc1cnc(N2CC[C@H](N(C)C(=O)Nc3cc(C(F)(F)F)cn(C)c3=O)C(F)(F)C2)cn1 ; Cc1n[nH]c2ncc(N3CCC(N(C)C(=O)Nc4cc(C(F)(F)F)cn(C)c4=O)C(F)(F)C3)cc12 ; Cc1n[nH]c2ncc(N3CC[C@@H](N(C)C(=O)Nc4cc(C(F)(F)F)cn(C)c4=O)C(F)(F)C3)cc12] 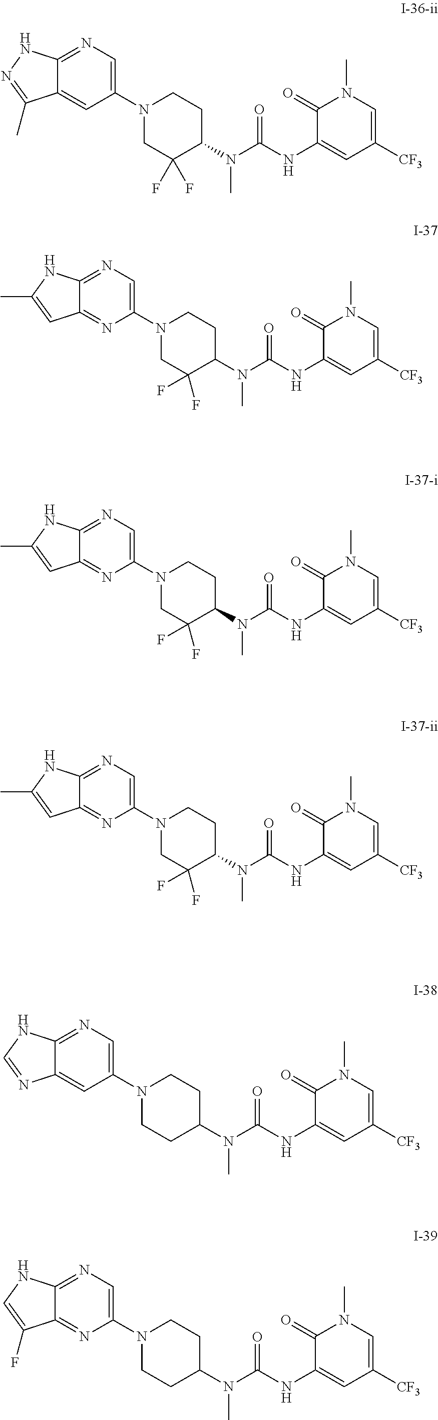 [SMILES: Cc1n[nH]c2ncc(N3CC[C@H](N(C)C(=O)Nc4cc(C(F)(F)F)cn(C)c4=O)C(F)(F)C3)cc12 ; Cc1cc2nc(N3CCC(N(C)C(=O)Nc4cc(C(F)(F)F)cn(C)c4=O)C(F)(F)C3)cnc2[nH]1 ; Cc1cc2nc(N3CC[C@@H](N(C)C(=O)Nc4cc(C(F)(F)F)cn(C)c4=O)C(F)(F)C3)cnc2[nH]1 ; Cc1cc2nc(N3CC[C@H](N(C)C(=O)Nc4cc(C(F)(F)F)cn(C)c4=O)C(F)(F)C3)cnc2[nH]1 ; CN(C(=O)Nc1cc(C(F)(F)F)cn(C)c1=O)C1CCN(c2cnc3[nH]cnc3c2)CC1 ; CN(C(=O)Nc1cc(C(F)(F)F)cn(C)c1=O)C1CCN(c2cnc3[nH]cc(F)c3n2)CC1] 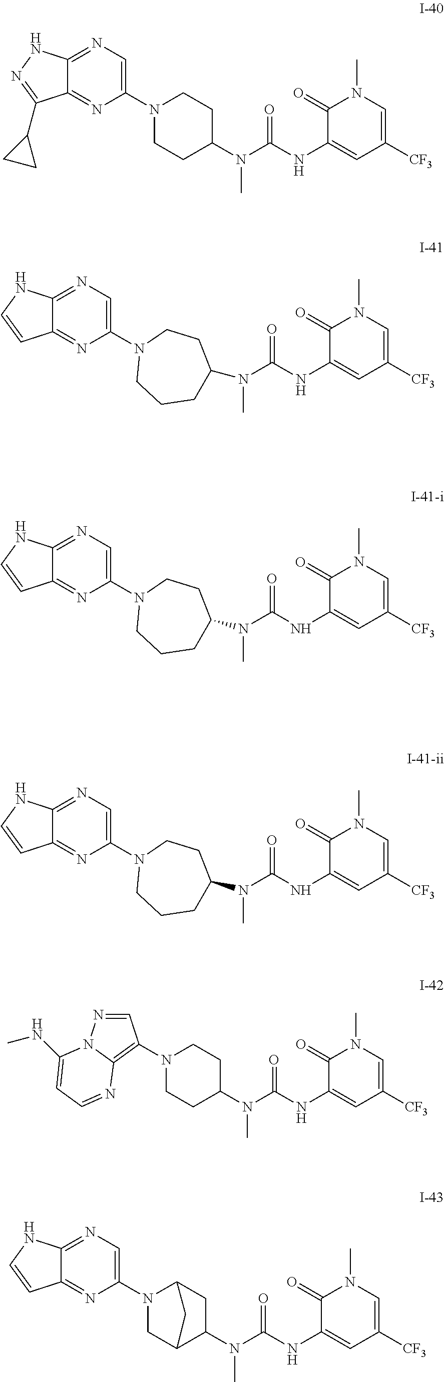 [SMILES: CN(C(=O)Nc1cc(C(F)(F)F)cn(C)c1=O)C1CCN(c2cnc3[nH]nc(C4CC4)c3n2)CC1 ; CN(C(=O)Nc1cc(C(F)(F)F)cn(C)c1=O)C1CCCN(c2cnc3[nH]ccc3n2)CC1 ; CN(C(=O)Nc1cc(C(F)(F)F)cn(C)c1=O)[C@@H]1CCCN(c2cnc3[nH]ccc3n2)CC1 ; CN(C(=O)Nc1cc(C(F)(F)F)cn(C)c1=O)[C@H]1CCCN(c2cnc3[nH]ccc3n2)CC1 ; CNc1ccnc2c(N3CCC(N(C)C(=O)Nc4cc(C(F)(F)F)cn(C)c4=O)CC3)cnn12 ; CN(C(=O)Nc1cc(C(F)(F)F)cn(C)c1=O)C1CC2CC1CN2c1cnc2[nH]ccc2n1] 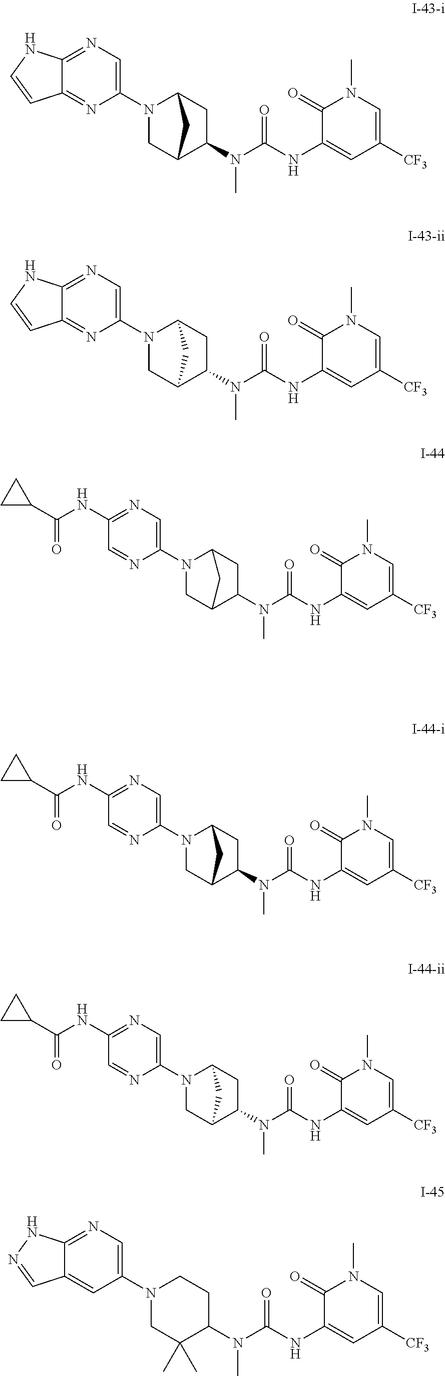 [SMILES: CN(C(=O)Nc1cc(C(F)(F)F)cn(C)c1=O)[C@@H]1C[C@@H]2C[C@H]1CN2c1cnc2[nH]ccc2n1 ; CN(C(=O)Nc1cc(C(F)(F)F)cn(C)c1=O)[C@H]1C[C@H]2C[C@@H]1CN2c1cnc2[nH]ccc2n1 ; CN(C(=O)Nc1cc(C(F)(F)F)cn(C)c1=O)C1CC2CC1CN2c1cnc(NC(=O)C2CC2)cn1 ; CN(C(=O)Nc1cc(C(F)(F)F)cn(C)c1=O)[C@@H]1C[C@@H]2C[C@H]1CN2c1cnc(NC(=O)C2CC2)cn1 ; CN(C(=O)Nc1cc(C(F)(F)F)cn(C)c1=O)[C@H]1C[C@H]2C[C@@H]1CN2c1cnc(NC(=O)C2CC2)cn1 ; CN(C(=O)Nc1cc(C(F)(F)F)cn(C)c1=O)C1CCN(c2cnc3[nH]ncc3c2)CC1(C)C] 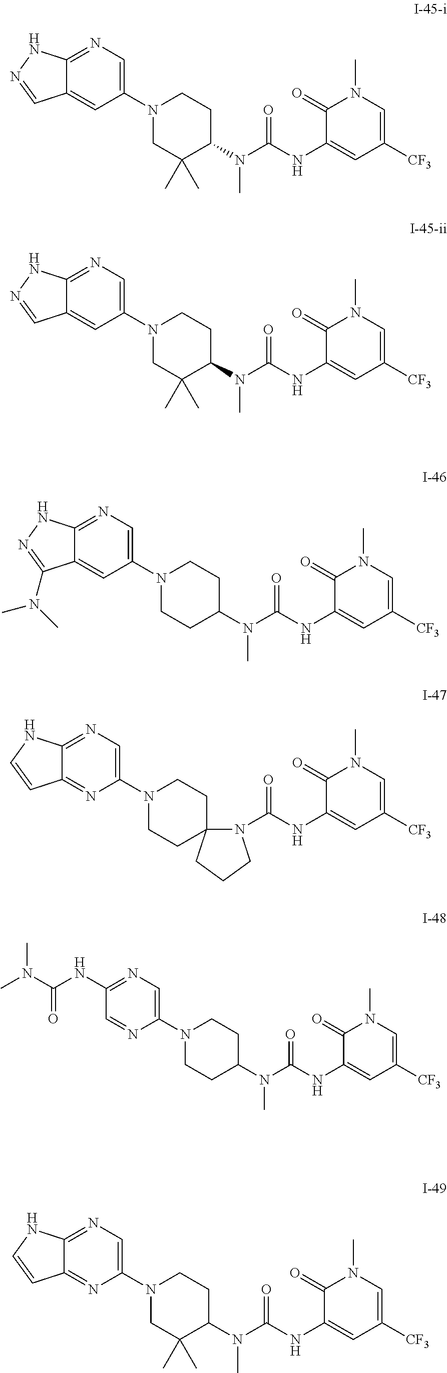 [SMILES: CN(C(=O)Nc1cc(C(F)(F)F)cn(C)c1=O)[C@H]1CCN(c2cnc3[nH]ncc3c2)CC1(C)C ; CN(C(=O)Nc1cc(C(F)(F)F)cn(C)c1=O)[C@@H]1CCN(c2cnc3[nH]ncc3c2)CC1(C)C ; CN(C)c1n[nH]c2ncc(N3CCC(N(C)C(=O)Nc4cc(C(F)(F)F)cn(C)c4=O)CC3)cc12 ; Cn1cc(C(F)(F)F)cc(NC(=O)N2CCCC23CCN(c2cnc4[nH]ccc4n2)CC3)c1=O ; CN(C)C(=O)Nc1cnc(N2CCC(N(C)C(=O)Nc3cc(C(F)(F)F)cn(C)c3=O)CC2)cn1 ; CN(C(=O)Nc1cc(C(F)(F)F)cn(C)c1=O)C1CCN(c2cnc3[nH]ccc3n2)CC1(C)C] 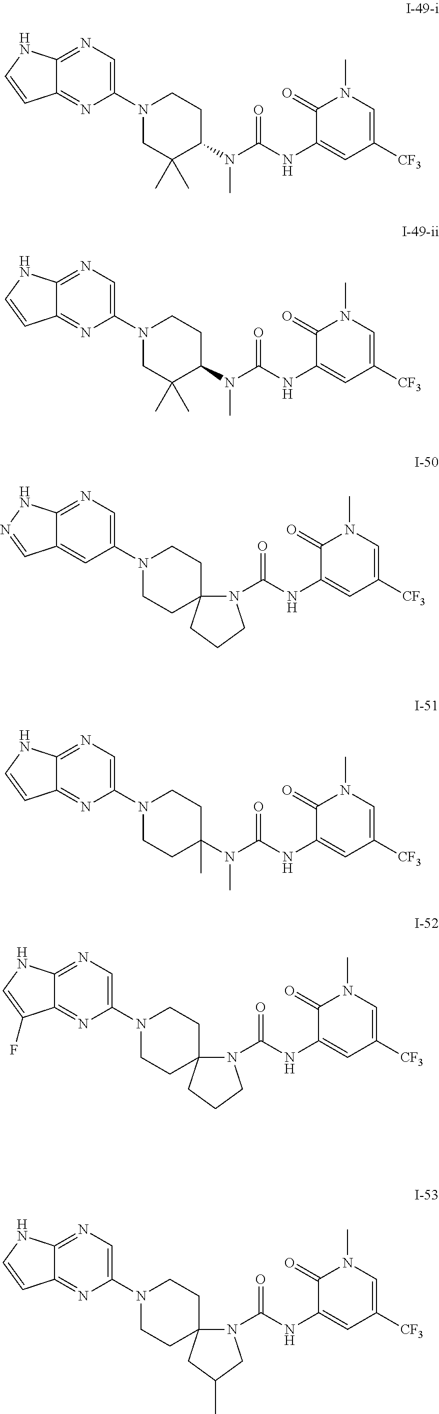 [SMILES: CN(C(=O)Nc1cc(C(F)(F)F)cn(C)c1=O)[C@H]1CCN(c2cnc3[nH]ccc3n2)CC1(C)C ; CN(C(=O)Nc1cc(C(F)(F)F)cn(C)c1=O)[C@@H]1CCN(c2cnc3[nH]ccc3n2)CC1(C)C ; Cn1cc(C(F)(F)F)cc(NC(=O)N2CCCC23CCN(c2cnc4[nH]ncc4c2)CC3)c1=O ; CN(C(=O)Nc1cc(C(F)(F)F)cn(C)c1=O)C1(C)CCN(c2cnc3[nH]ccc3n2)CC1 ; Cn1cc(C(F)(F)F)cc(NC(=O)N2CCCC23CCN(c2cnc4[nH]cc(F)c4n2)CC3)c1=O ; CC1CN(C(=O)Nc2cc(C(F)(F)F)cn(C)c2=O)C2(CCN(c3cnc4[nH]ccc4n3)CC2)C1] 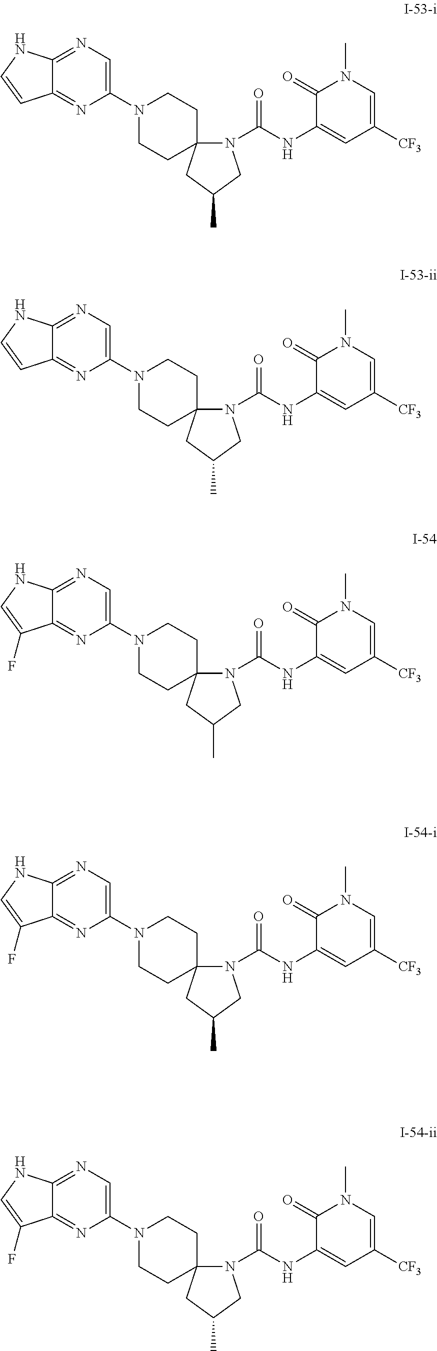 [SMILES: C[C@@H]1CN(C(=O)Nc2cc(C(F)(F)F)cn(C)c2=O)C2(CCN(c3cnc4[nH]ccc4n3)CC2)C1 ; C[C@H]1CN(C(=O)Nc2cc(C(F)(F)F)cn(C)c2=O)C2(CCN(c3cnc4[nH]ccc4n3)CC2)C1 ; CC1CN(C(=O)Nc2cc(C(F)(F)F)cn(C)c2=O)C2(CCN(c3cnc4[nH]cc(F)c4n3)CC2)C1 ; C[C@@H]1CN(C(=O)Nc2cc(C(F)(F)F)cn(C)c2=O)C2(CCN(c3cnc4[nH]cc(F)c4n3)CC2)C1 ; C[C@H]1CN(C(=O)Nc2cc(C(F)(F)F)cn(C)c2=O)C2(CCN(c3cnc4[nH]cc(F)c4n3)CC2)C1] 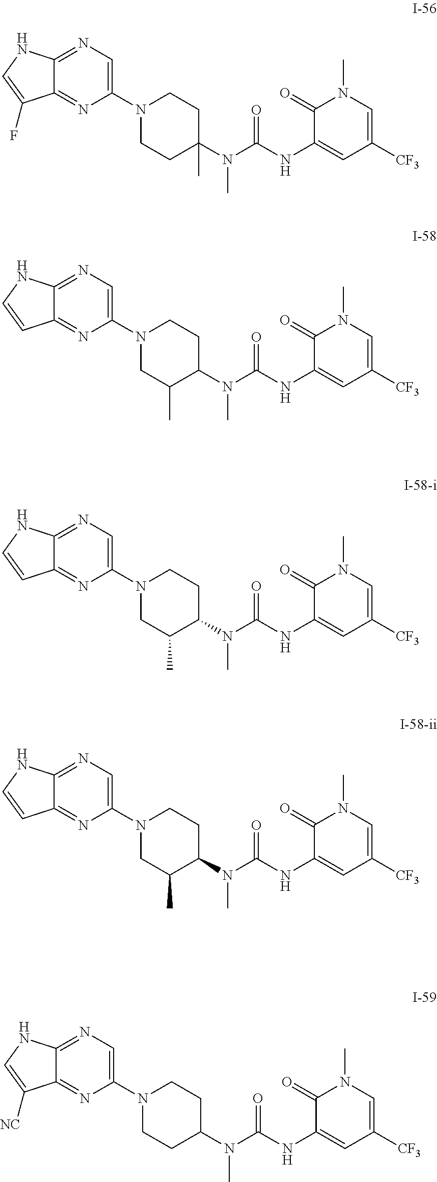 [SMILES: CN(C(=O)Nc1cc(C(F)(F)F)cn(C)c1=O)C1(C)CCN(c2cnc3[nH]cc(F)c3n2)CC1 ; CC1CN(c2cnc3[nH]ccc3n2)CCC1N(C)C(=O)Nc1cc(C(F)(F)F)cn(C)c1=O ; C[C@@H]1CN(c2cnc3[nH]ccc3n2)CC[C@@H]1N(C)C(=O)Nc1cc(C(F)(F)F)cn(C)c1=O ; C[C@H]1CN(c2cnc3[nH]ccc3n2)CC[C@H]1N(C)C(=O)Nc1cc(C(F)(F)F)cn(C)c1=O ; CN(C(=O)Nc1cc(C(F)(F)F)cn(C)c1=O)C1CCN(c2cnc3[nH]cc(C#N)c3n2)CC1] 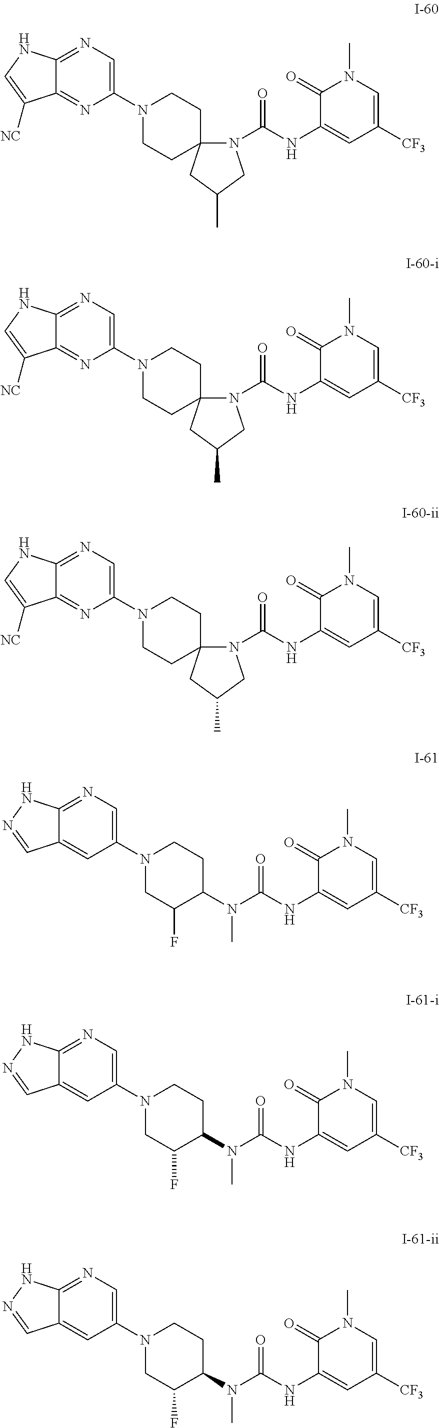 [SMILES: CC1CN(C(=O)Nc2cc(C(F)(F)F)cn(C)c2=O)C2(CCN(c3cnc4[nH]cc(C#N)c4n3)CC2)C1 ; C[C@@H]1CN(C(=O)Nc2cc(C(F)(F)F)cn(C)c2=O)C2(CCN(c3cnc4[nH]cc(C#N)c4n3)CC2)C1 ; C[C@H]1CN(C(=O)Nc2cc(C(F)(F)F)cn(C)c2=O)C2(CCN(c3cnc4[nH]cc(C#N)c4n3)CC2)C1 ; CN(C(=O)Nc1cc(C(F)(F)F)cn(C)c1=O)C1CCN(c2cnc3[nH]ncc3c2)CC1F ; CN(C(=O)Nc1cc(C(F)(F)F)cn(C)c1=O)[C@@H]1CCN(c2cnc3[nH]ncc3c2)C[C@H]1F ; CN(C(=O)Nc1cc(C(F)(F)F)cn(C)c1=O)[C@@H]1CCN(c2cnc3[nH]ncc3c2)C[C@H]1F] 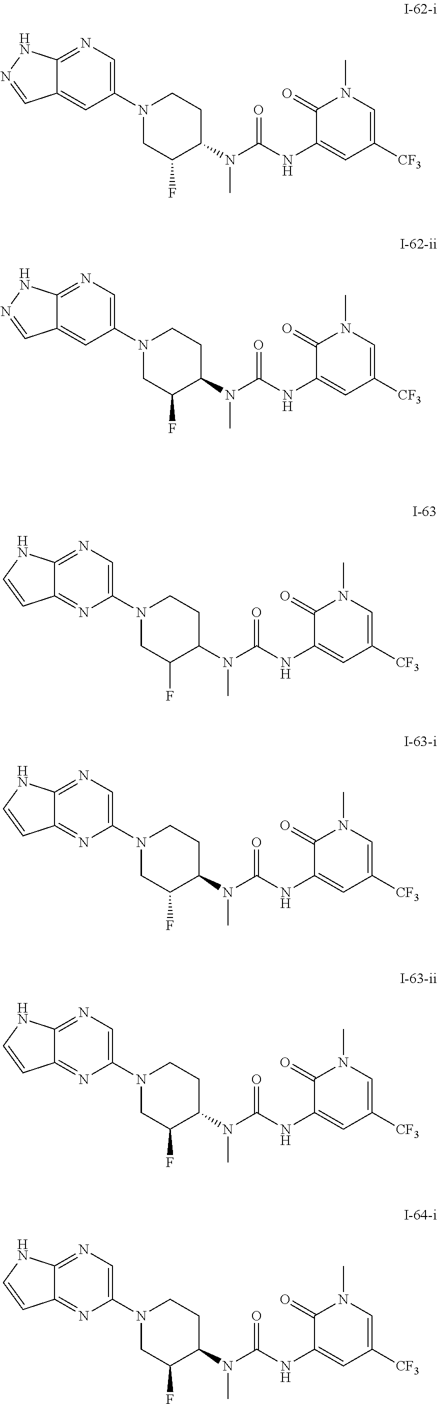 [SMILES: CN(C(=O)Nc1cc(C(F)(F)F)cn(C)c1=O)[C@H]1CCN(c2cnc3[nH]ncc3c2)C[C@H]1F ; CN(C(=O)Nc1cc(C(F)(F)F)cn(C)c1=O)[C@@H]1CCN(c2cnc3[nH]ncc3c2)C[C@@H]1F ; CN(C(=O)Nc1cc(C(F)(F)F)cn(C)c1=O)C1CCN(c2cnc3[nH]ccc3n2)CC1F ; CN(C(=O)Nc1cc(C(F)(F)F)cn(C)c1=O)[C@@H]1CCN(c2cnc3[nH]ccc3n2)C[C@H]1F ; CN(C(=O)Nc1cc(C(F)(F)F)cn(C)c1=O)[C@H]1CCN(c2cnc3[nH]ccc3n2)C[C@@H]1F ; CN(C(=O)Nc1cc(C(F)(F)F)cn(C)c1=O)[C@@H]1CCN(c2cnc3[nH]ccc3n2)C[C@@H]1F] 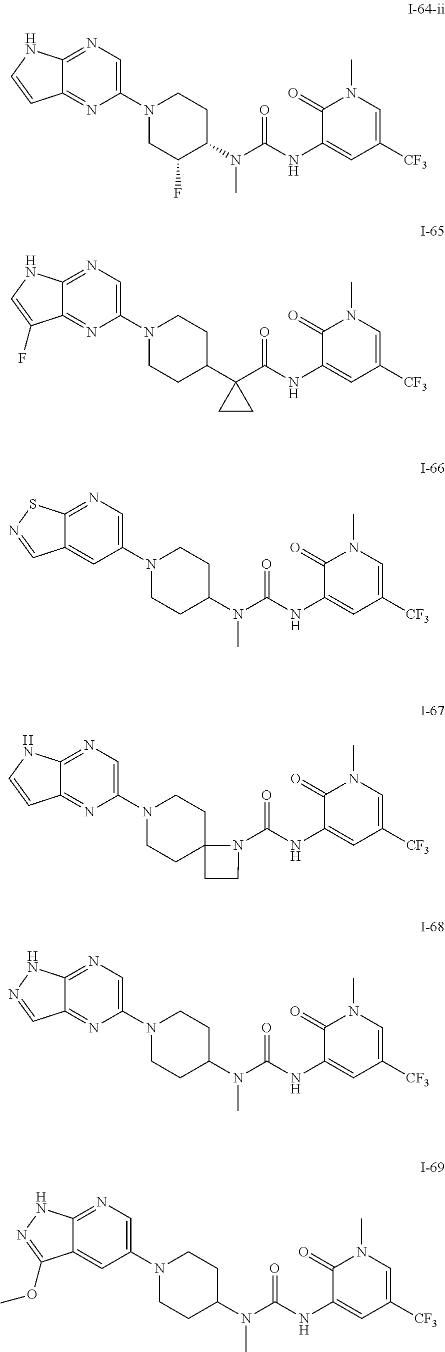 [SMILES: CN(C(=O)Nc1cc(C(F)(F)F)cn(C)c1=O)[C@H]1CCN(c2cnc3[nH]ccc3n2)C[C@H]1F ; Cn1cc(C(F)(F)F)cc(NC(=O)C2(C3CCN(c4cnc5[nH]cc(F)c5n4)CC3)CC2)c1=O ; CN(C(=O)Nc1cc(C(F)(F)F)cn(C)c1=O)C1CCN(c2cnc3sncc3c2)CC1 ; Cn1cc(C(F)(F)F)cc(NC(=O)N2CCC23CCN(c2cnc4[nH]ccc4n2)CC3)c1=O ; CN(C(=O)Nc1cc(C(F)(F)F)cn(C)c1=O)C1CCN(c2cnc3[nH]ncc3n2)CC1 ; COc1n[nH]c2ncc(N3CCC(N(C)C(=O)Nc4cc(C(F)(F)F)cn(C)c4=O)CC3)cc12] 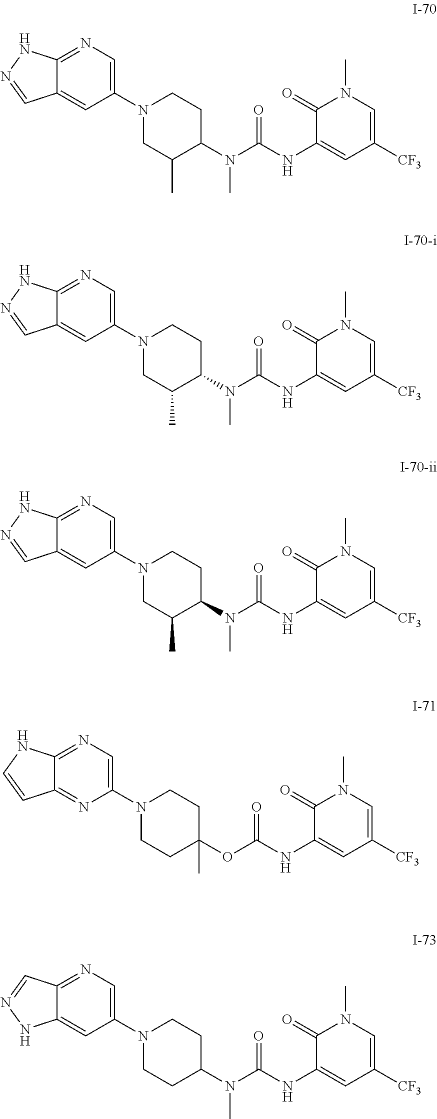 [SMILES: CC1CN(c2cnc3[nH]ncc3c2)CCC1N(C)C(=O)Nc1cc(C(F)(F)F)cn(C)c1=O ; C[C@@H]1CN(c2cnc3[nH]ncc3c2)CC[C@@H]1N(C)C(=O)Nc1cc(C(F)(F)F)cn(C)c1=O ; C[C@H]1CN(c2cnc3[nH]ncc3c2)CC[C@H]1N(C)C(=O)Nc1cc(C(F)(F)F)cn(C)c1=O ; Cn1cc(C(F)(F)F)cc(NC(=O)OC2(C)CCN(c3cnc4[nH]ccc4n3)CC2)c1=O ; CN(C(=O)Nc1cc(C(F)(F)F)cn(C)c1=O)C1CCN(c2cnc3cn[nH]c3c2)CC1] 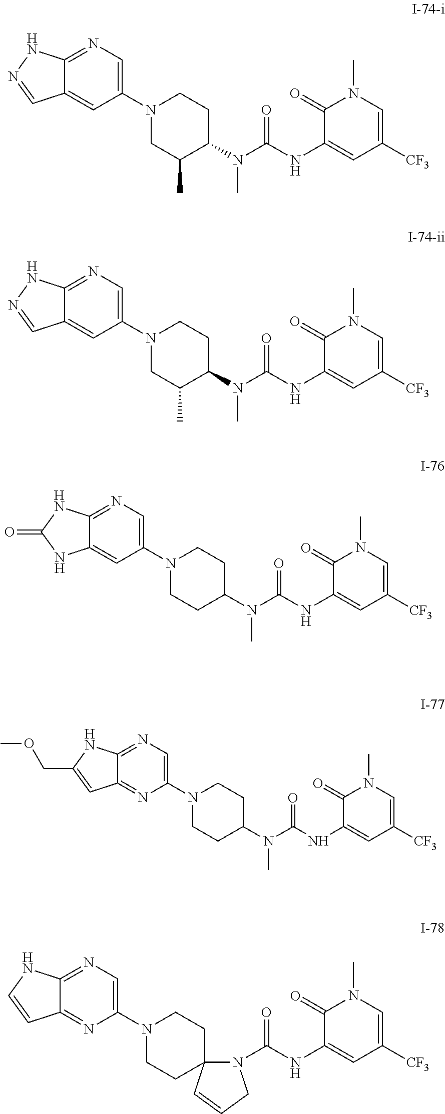 [SMILES: C[C@H]1CN(c2cnc3[nH]ncc3c2)CC[C@@H]1N(C)C(=O)Nc1cc(C(F)(F)F)cn(C)c1=O ; C[C@@H]1CN(c2cnc3[nH]ncc3c2)CC[C@H]1N(C)C(=O)Nc1cc(C(F)(F)F)cn(C)c1=O ; CN(C(=O)Nc1cc(C(F)(F)F)cn(C)c1=O)C1CCN(c2cnc3[nH]c(=O)[nH]c3c2)CC1 ; COCc1cc2nc(N3CCC(N(C)C(=O)Nc4cc(C(F)(F)F)cn(C)c4=O)CC3)cnc2[nH]1 ; Cn1cc(C(F)(F)F)cc(NC(=O)N2CC=CC23CCN(c2cnc4[nH]ccc4n2)CC3)c1=O] 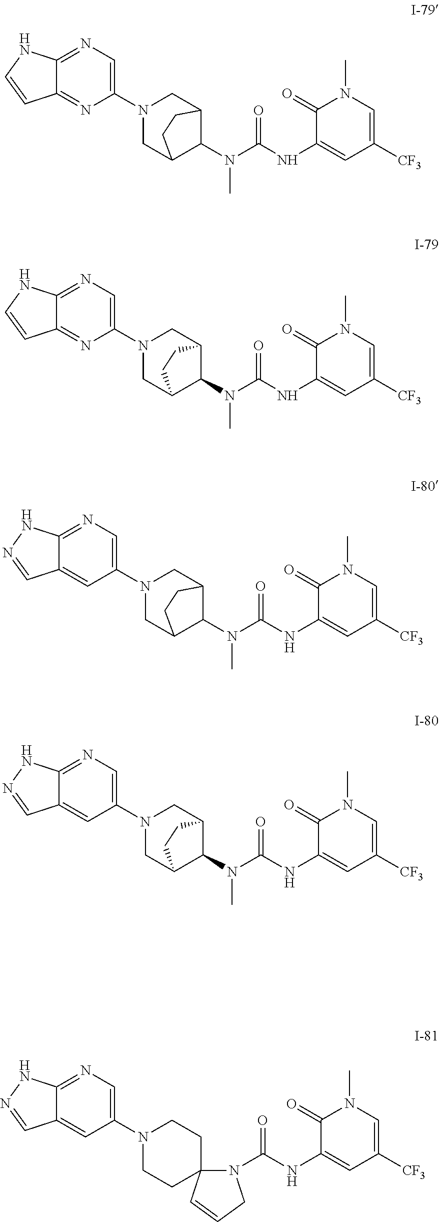 [SMILES: CN(C(=O)Nc1cc(C(F)(F)F)cn(C)c1=O)C1C2CCC1CN(c1cnc3[nH]ccc3n1)C2 ; CN(C(=O)Nc1cc(C(F)(F)F)cn(C)c1=O)[C@H]1[C@@H]2CC[C@H]1CN(c1cnc3[nH]ccc3n1)C2 ; CN(C(=O)Nc1cc(C(F)(F)F)cn(C)c1=O)C1C2CCC1CN(c1cnc3[nH]ncc3c1)C2 ; CN(C(=O)Nc1cc(C(F)(F)F)cn(C)c1=O)[C@H]1[C@@H]2CC[C@H]1CN(c1cnc3[nH]ncc3c1)C2 ; Cn1cc(C(F)(F)F)cc(NC(=O)N2CC=CC23CCN(c2cnc4[nH]ncc4c2)CC3)c1=O] 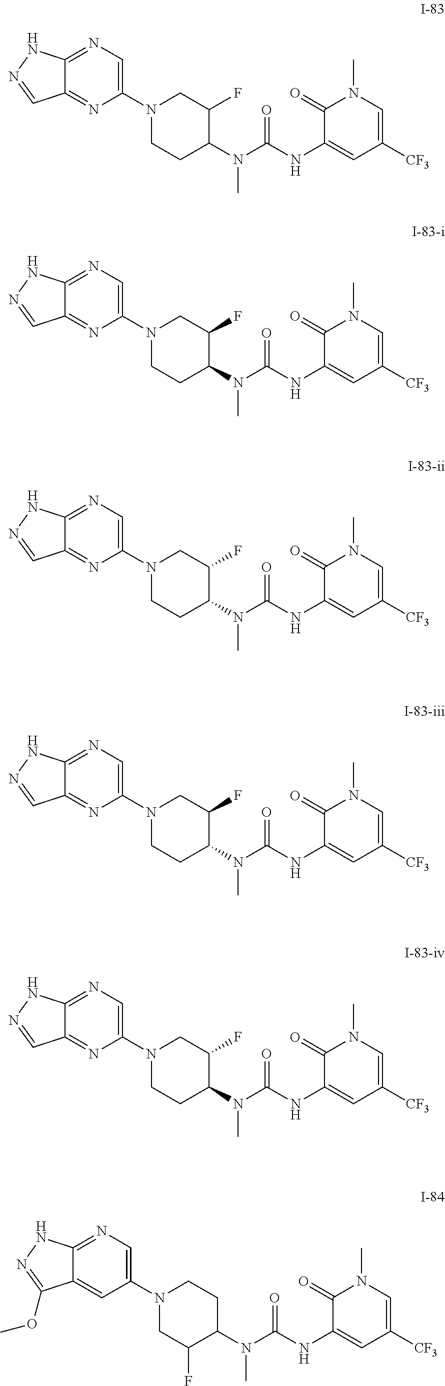 [SMILES: CN(C(=O)Nc1cc(C(F)(F)F)cn(C)c1=O)C1CCN(c2cnc3[nH]ncc3n2)CC1F ; CN(C(=O)Nc1cc(C(F)(F)F)cn(C)c1=O)[C@H]1CCN(c2cnc3[nH]ncc3n2)C[C@H]1F ; CN(C(=O)Nc1cc(C(F)(F)F)cn(C)c1=O)[C@@H]1CCN(c2cnc3[nH]ncc3n2)C[C@@H]1F ; CN(C(=O)Nc1cc(C(F)(F)F)cn(C)c1=O)[C@@H]1CCN(c2cnc3[nH]ncc3n2)C[C@H]1F ; CN(C(=O)Nc1cc(C(F)(F)F)cn(C)c1=O)[C@H]1CCN(c2cnc3[nH]ncc3n2)C[C@@H]1F ; COc1n[nH]c2ncc(N3CCC(N(C)C(=O)Nc4cc(C(F)(F)F)cn(C)c4=O)C(F)C3)cc12] 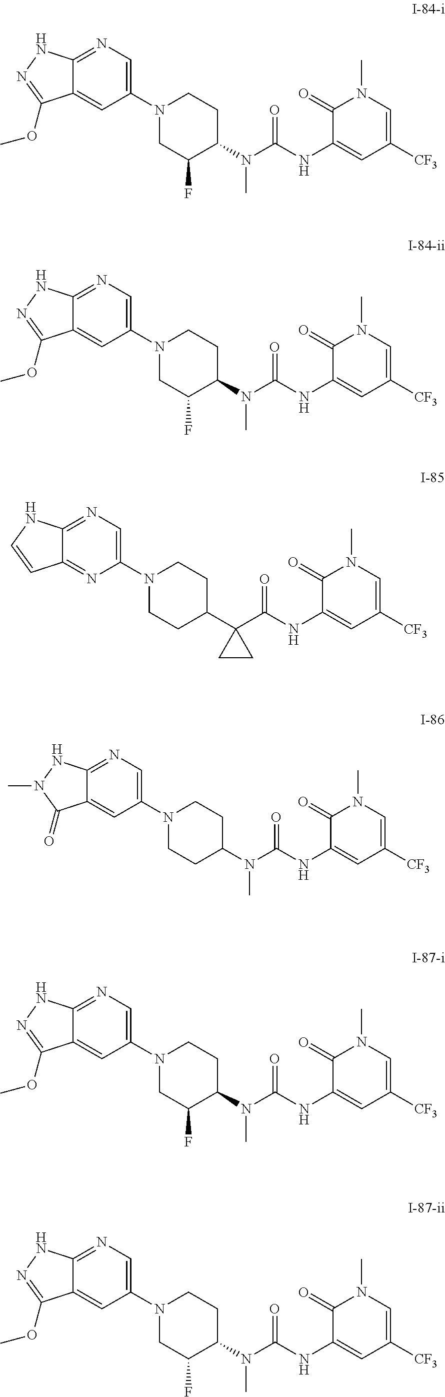 [SMILES: COc1n[nH]c2ncc(N3CC[C@H](N(C)C(=O)Nc4cc(C(F)(F)F)cn(C)c4=O)[C@@H](F)C3)cc12 ; COc1n[nH]c2ncc(N3CC[C@@H](N(C)C(=O)Nc4cc(C(F)(F)F)cn(C)c4=O)[C@H](F)C3)cc12 ; Cn1cc(C(F)(F)F)cc(NC(=O)C2(C3CCN(c4cnc5[nH]ccc5n4)CC3)CC2)c1=O ; CN(C(=O)Nc1cc(C(F)(F)F)cn(C)c1=O)C1CCN(c2cnc3[nH]n(C)c(=O)c3c2)CC1 ; COc1n[nH]c2ncc(N3CC[C@@H](N(C)C(=O)Nc4cc(C(F)(F)F)cn(C)c4=O)[C@@H](F)C3)cc12 ; COc1n[nH]c2ncc(N3CC[C@H](N(C)C(=O)Nc4cc(C(F)(F)F)cn(C)c4=O)[C@H](F)C3)cc12] 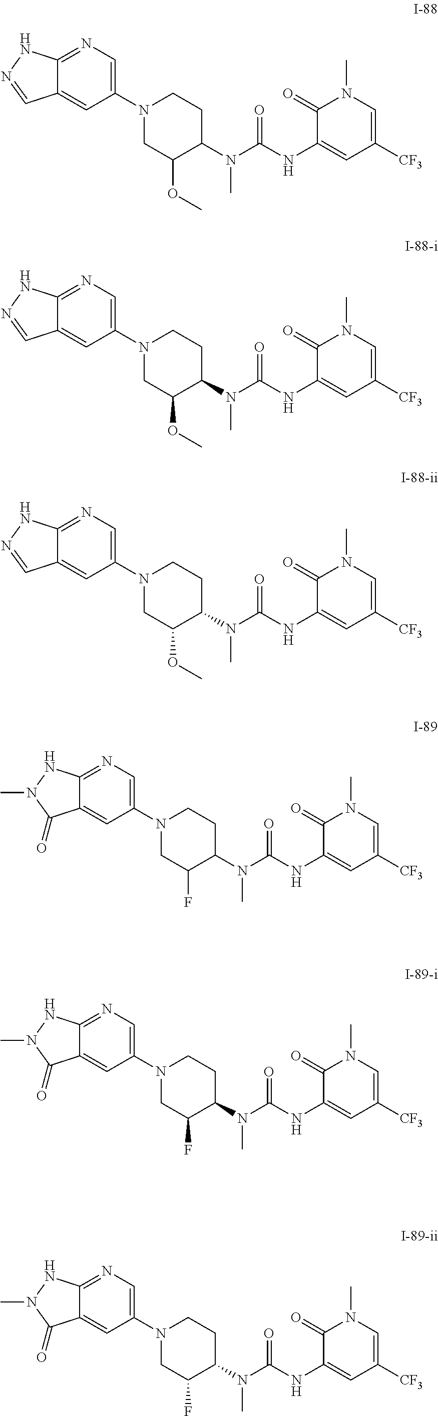 [SMILES: COC1CN(c2cnc3[nH]ncc3c2)CCC1N(C)C(=O)Nc1cc(C(F)(F)F)cn(C)c1=O ; CO[C@H]1CN(c2cnc3[nH]ncc3c2)CC[C@H]1N(C)C(=O)Nc1cc(C(F)(F)F)cn(C)c1=O ; CO[C@@H]1CN(c2cnc3[nH]ncc3c2)CC[C@@H]1N(C)C(=O)Nc1cc(C(F)(F)F)cn(C)c1=O ; CN(C(=O)Nc1cc(C(F)(F)F)cn(C)c1=O)C1CCN(c2cnc3[nH]n(C)c(=O)c3c2)CC1F ; CN(C(=O)Nc1cc(C(F)(F)F)cn(C)c1=O)[C@@H]1CCN(c2cnc3[nH]n(C)c(=O)c3c2)C[C@@H]1F ; CN(C(=O)Nc1cc(C(F)(F)F)cn(C)c1=O)[C@H]1CCN(c2cnc3[nH]n(C)c(=O)c3c2)C[C@H]1F] 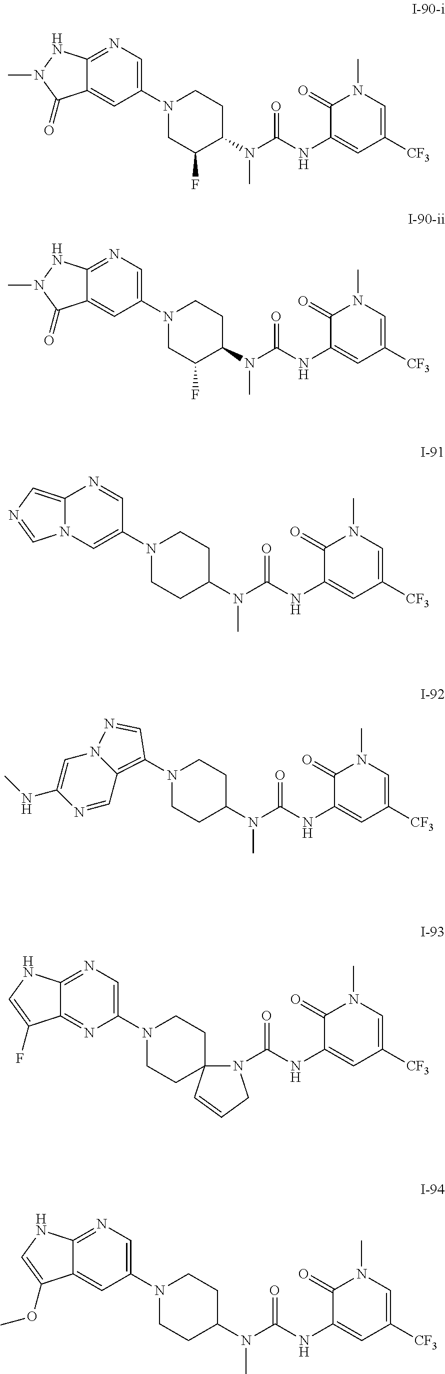 [SMILES: CN(C(=O)Nc1cc(C(F)(F)F)cn(C)c1=O)[C@H]1CCN(c2cnc3[nH]n(C)c(=O)c3c2)C[C@@H]1F ; CN(C(=O)Nc1cc(C(F)(F)F)cn(C)c1=O)[C@@H]1CCN(c2cnc3[nH]n(C)c(=O)c3c2)C[C@H]1F ; CN(C(=O)Nc1cc(C(F)(F)F)cn(C)c1=O)C1CCN(c2cnc3cncn3c2)CC1 ; CNc1cn2ncc(N3CCC(N(C)C(=O)Nc4cc(C(F)(F)F)cn(C)c4=O)CC3)c2cn1 ; Cn1cc(C(F)(F)F)cc(NC(=O)N2CC=CC23CCN(c2cnc4[nH]cc(F)c4n2)CC3)c1=O ; COc1c[nH]c2ncc(N3CCC(N(C)C(=O)Nc4cc(C(F)(F)F)cn(C)c4=O)CC3)cc12] 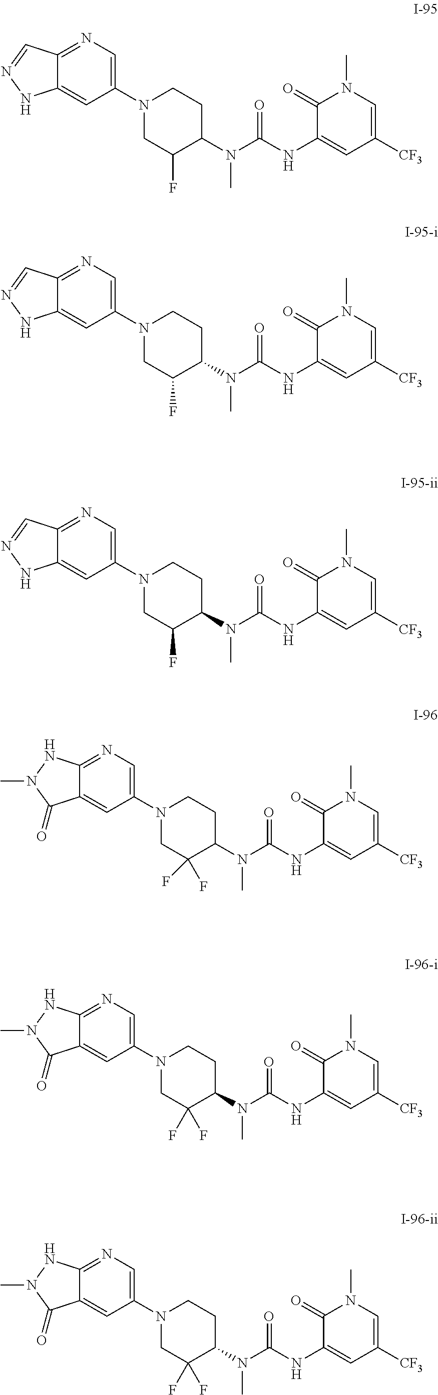 [SMILES: CN(C(=O)Nc1cc(C(F)(F)F)cn(C)c1=O)C1CCN(c2cnc3cn[nH]c3c2)CC1F ; CN(C(=O)Nc1cc(C(F)(F)F)cn(C)c1=O)[C@H]1CCN(c2cnc3cn[nH]c3c2)C[C@H]1F ; CN(C(=O)Nc1cc(C(F)(F)F)cn(C)c1=O)[C@@H]1CCN(c2cnc3cn[nH]c3c2)C[C@@H]1F ; CN(C(=O)Nc1cc(C(F)(F)F)cn(C)c1=O)C1CCN(c2cnc3[nH]n(C)c(=O)c3c2)CC1(F)F ; CN(C(=O)Nc1cc(C(F)(F)F)cn(C)c1=O)[C@@H]1CCN(c2cnc3[nH]n(C)c(=O)c3c2)CC1(F)F ; CN(C(=O)Nc1cc(C(F)(F)F)cn(C)c1=O)[C@H]1CCN(c2cnc3[nH]n(C)c(=O)c3c2)CC1(F)F] 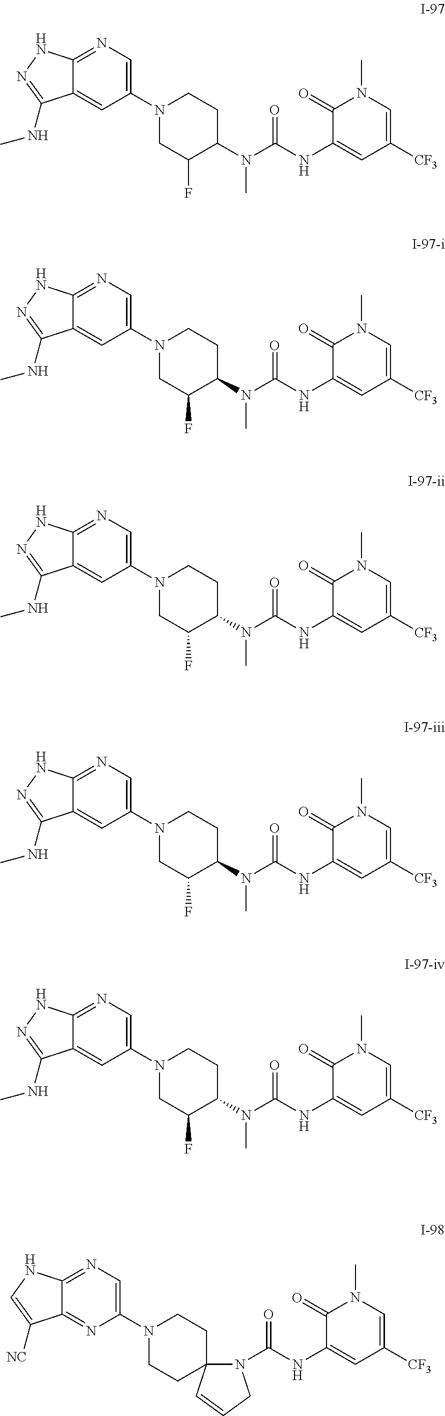 [SMILES: CNc1n[nH]c2ncc(N3CCC(N(C)C(=O)Nc4cc(C(F)(F)F)cn(C)c4=O)C(F)C3)cc12 ; CNc1n[nH]c2ncc(N3CC[C@@H](N(C)C(=O)Nc4cc(C(F)(F)F)cn(C)c4=O)[C@@H](F)C3)cc12 ; CNc1n[nH]c2ncc(N3CC[C@H](N(C)C(=O)Nc4cc(C(F)(F)F)cn(C)c4=O)[C@H](F)C3)cc12 ; CNc1n[nH]c2ncc(N3CC[C@@H](N(C)C(=O)Nc4cc(C(F)(F)F)cn(C)c4=O)[C@H](F)C3)cc12 ; CNc1n[nH]c2ncc(N3CC[C@H](N(C)C(=O)Nc4cc(C(F)(F)F)cn(C)c4=O)[C@@H](F)C3)cc12 ; Cn1cc(C(F)(F)F)cc(NC(=O)N2CC=CC23CCN(c2cnc4[nH]cc(C#N)c4n2)CC3)c1=O] 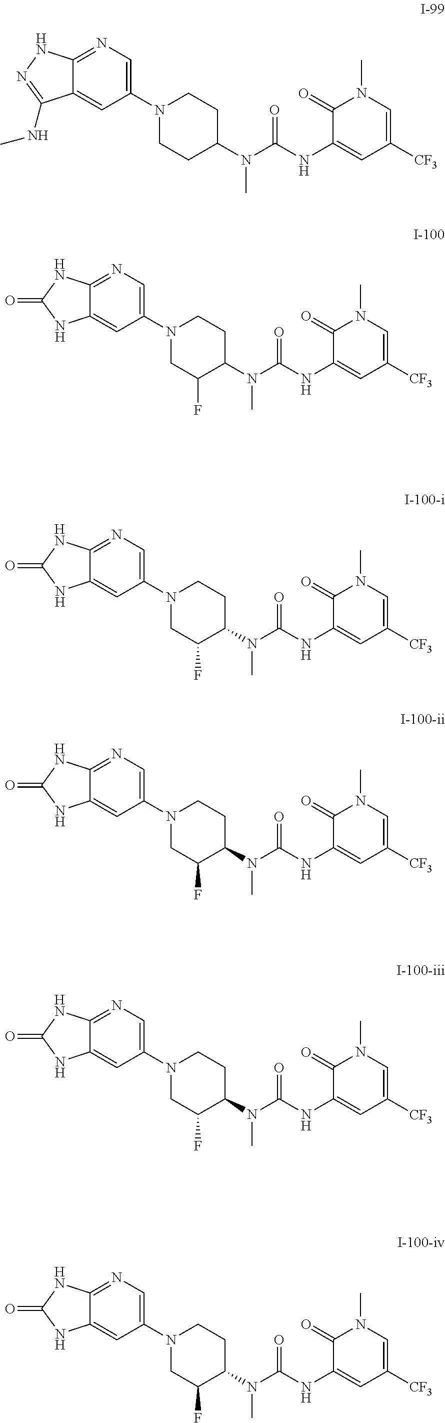 [SMILES: CNc1n[nH]c2ncc(N3CCC(N(C)C(=O)Nc4cc(C(F)(F)F)cn(C)c4=O)CC3)cc12 ; CN(C(=O)Nc1cc(C(F)(F)F)cn(C)c1=O)C1CCN(c2cnc3[nH]c(=O)[nH]c3c2)CC1F ; CN(C(=O)Nc1cc(C(F)(F)F)cn(C)c1=O)[C@H]1CCN(c2cnc3[nH]c(=O)[nH]c3c2)C[C@H]1F ; CN(C(=O)Nc1cc(C(F)(F)F)cn(C)c1=O)[C@@H]1CCN(c2cnc3[nH]c(=O)[nH]c3c2)C[C@@H]1F ; CN(C(=O)Nc1cc(C(F)(F)F)cn(C)c1=O)[C@@H]1CCN(c2cnc3[nH]c(=O)[nH]c3c2)C[C@H]1F ; CN(C(=O)Nc1cc(C(F)(F)F)cn(C)c1=O)[C@H]1CCN(c2cnc3[nH]c(=O)[nH]c3c2)C[C@@H]1F] 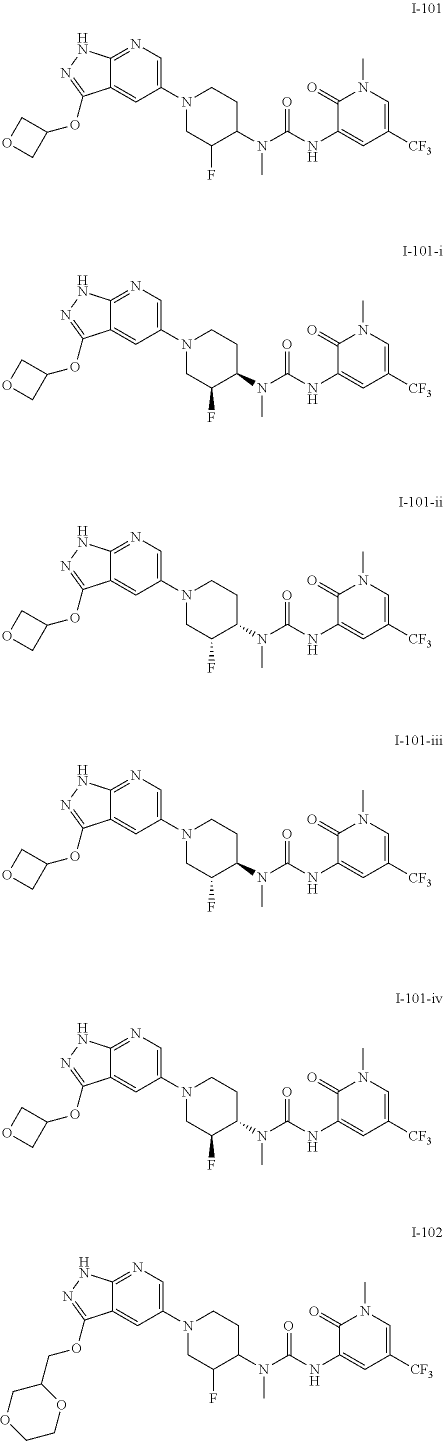 [SMILES: CN(C(=O)Nc1cc(C(F)(F)F)cn(C)c1=O)C1CCN(c2cnc3[nH]nc(OC4COC4)c3c2)CC1F ; CN(C(=O)Nc1cc(C(F)(F)F)cn(C)c1=O)[C@@H]1CCN(c2cnc3[nH]nc(OC4COC4)c3c2)C[C@@H]1F ; CN(C(=O)Nc1cc(C(F)(F)F)cn(C)c1=O)[C@H]1CCN(c2cnc3[nH]nc(OC4COC4)c3c2)C[C@H]1F ; CN(C(=O)Nc1cc(C(F)(F)F)cn(C)c1=O)[C@@H]1CCN(c2cnc3[nH]nc(OC4COC4)c3c2)C[C@H]1F ; CN(C(=O)Nc1cc(C(F)(F)F)cn(C)c1=O)[C@H]1CCN(c2cnc3[nH]nc(OC4COC4)c3c2)C[C@@H]1F ; CN(C(=O)Nc1cc(C(F)(F)F)cn(C)c1=O)C1CCN(c2cnc3[nH]nc(OCC4COCCO4)c3c2)CC1F] 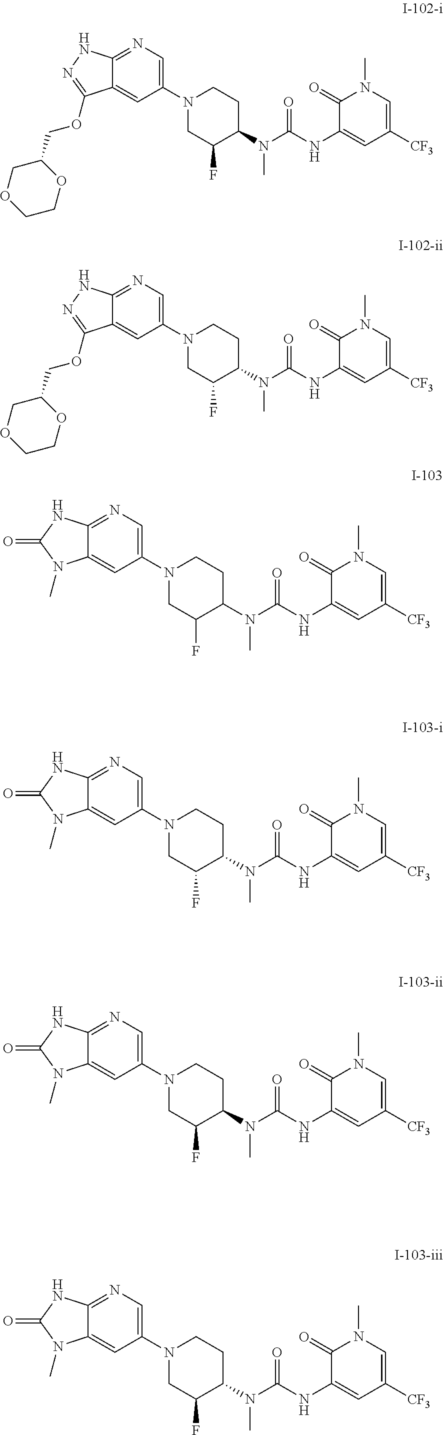 [SMILES: CN(C(=O)Nc1cc(C(F)(F)F)cn(C)c1=O)[C@@H]1CCN(c2cnc3[nH]nc(OC[C@H]4COCCO4)c3c2)C[C@@H]1F ; CN(C(=O)Nc1cc(C(F)(F)F)cn(C)c1=O)[C@H]1CCN(c2cnc3[nH]nc(OC[C@H]4COCCO4)c3c2)C[C@H]1F ; CN(C(=O)Nc1cc(C(F)(F)F)cn(C)c1=O)C1CCN(c2cnc3[nH]c(=O)n(C)c3c2)CC1F ; CN(C(=O)Nc1cc(C(F)(F)F)cn(C)c1=O)[C@H]1CCN(c2cnc3[nH]c(=O)n(C)c3c2)C[C@H]1F ; CN(C(=O)Nc1cc(C(F)(F)F)cn(C)c1=O)[C@@H]1CCN(c2cnc3[nH]c(=O)n(C)c3c2)C[C@@H]1F ; CN(C(=O)Nc1cc(C(F)(F)F)cn(C)c1=O)[C@H]1CCN(c2cnc3[nH]c(=O)n(C)c3c2)C[C@@H]1F] 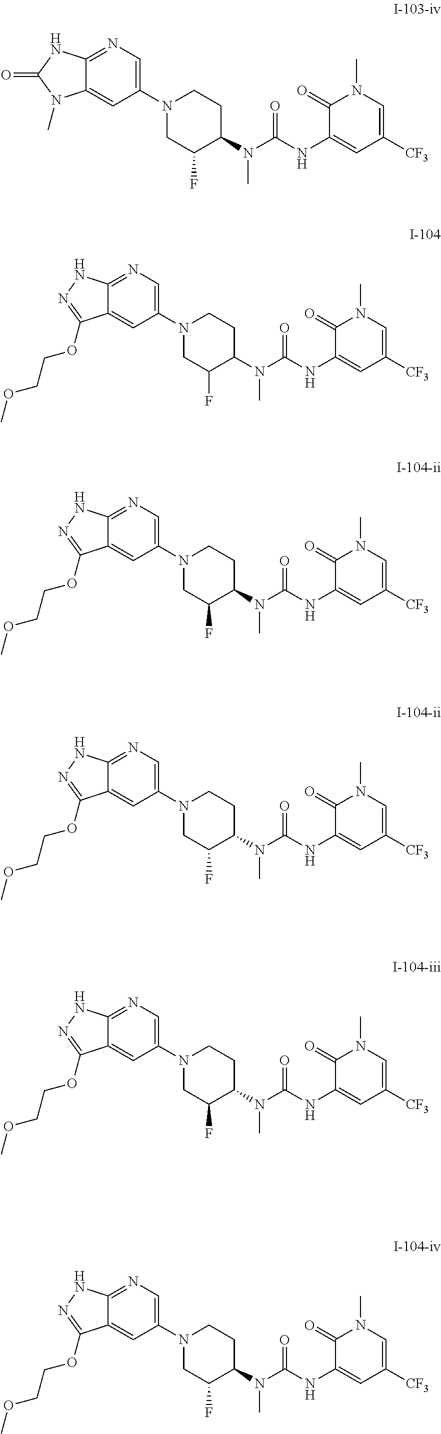 [SMILES: CN(C(=O)Nc1cc(C(F)(F)F)cn(C)c1=O)[C@@H]1CCN(c2cnc3[nH]c(=O)n(C)c3c2)C[C@H]1F ; COCCOc1n[nH]c2ncc(N3CCC(N(C)C(=O)Nc4cc(C(F)(F)F)cn(C)c4=O)C(F)C3)cc12 ; COCCOc1n[nH]c2ncc(N3CC[C@@H](N(C)C(=O)Nc4cc(C(F)(F)F)cn(C)c4=O)[C@@H](F)C3)cc12 ; COCCOc1n[nH]c2ncc(N3CC[C@H](N(C)C(=O)Nc4cc(C(F)(F)F)cn(C)c4=O)[C@H](F)C3)cc12 ; COCCOc1n[nH]c2ncc(N3CC[C@H](N(C)C(=O)Nc4cc(C(F)(F)F)cn(C)c4=O)[C@@H](F)C3)cc12 ; COCCOc1n[nH]c2ncc(N3CC[C@@H](N(C)C(=O)Nc4cc(C(F)(F)F)cn(C)c4=O)[C@H](F)C3)cc12] 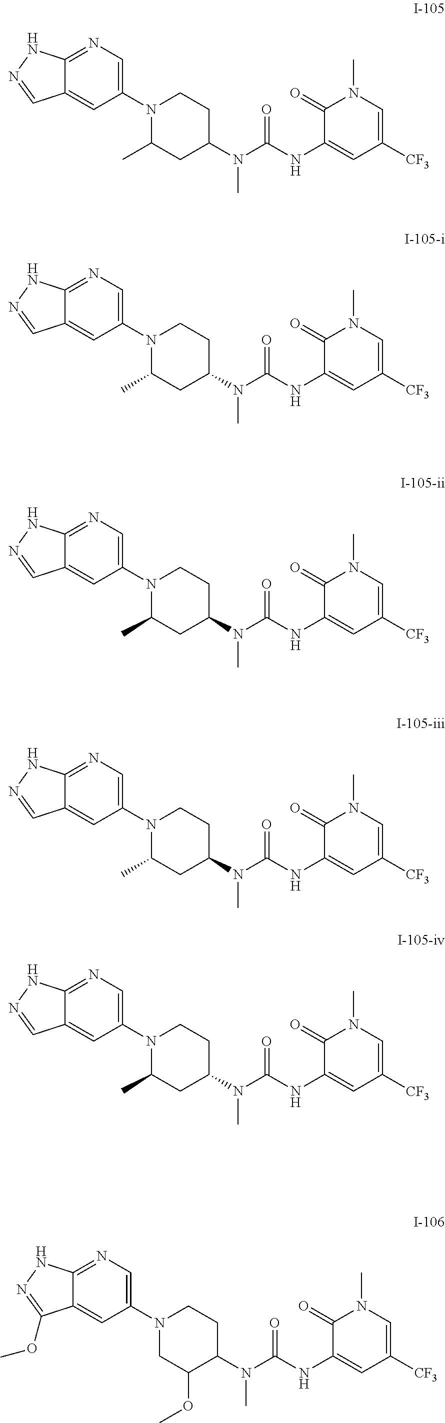 [SMILES: CC1CC(N(C)C(=O)Nc2cc(C(F)(F)F)cn(C)c2=O)CCN1c1cnc2[nH]ncc2c1 ; C[C@H]1C[C@@H](N(C)C(=O)Nc2cc(C(F)(F)F)cn(C)c2=O)CCN1c1cnc2[nH]ncc2c1 ; C[C@@H]1C[C@H](N(C)C(=O)Nc2cc(C(F)(F)F)cn(C)c2=O)CCN1c1cnc2[nH]ncc2c1 ; C[C@H]1C[C@H](N(C)C(=O)Nc2cc(C(F)(F)F)cn(C)c2=O)CCN1c1cnc2[nH]ncc2c1 ; C[C@@H]1C[C@@H](N(C)C(=O)Nc2cc(C(F)(F)F)cn(C)c2=O)CCN1c1cnc2[nH]ncc2c1 ; COc1n[nH]c2ncc(N3CCC(N(C)C(=O)Nc4cc(C(F)(F)F)cn(C)c4=O)C(OC)C3)cc12] 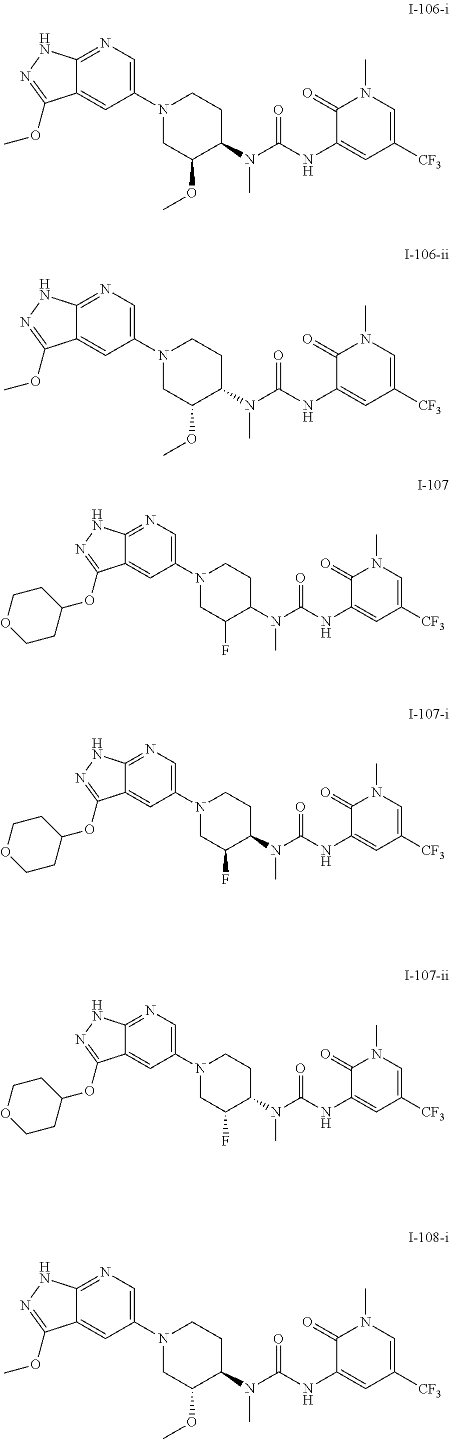 [SMILES: COc1n[nH]c2ncc(N3CC[C@@H](N(C)C(=O)Nc4cc(C(F)(F)F)cn(C)c4=O)[C@@H](OC)C3)cc12 ; COc1n[nH]c2ncc(N3CC[C@H](N(C)C(=O)Nc4cc(C(F)(F)F)cn(C)c4=O)[C@H](OC)C3)cc12 ; CN(C(=O)Nc1cc(C(F)(F)F)cn(C)c1=O)C1CCN(c2cnc3[nH]nc(OC4CCOCC4)c3c2)CC1F ; CN(C(=O)Nc1cc(C(F)(F)F)cn(C)c1=O)[C@@H]1CCN(c2cnc3[nH]nc(OC4CCOCC4)c3c2)C[C@@H]1F ; CN(C(=O)Nc1cc(C(F)(F)F)cn(C)c1=O)[C@H]1CCN(c2cnc3[nH]nc(OC4CCOCC4)c3c2)C[C@H]1F ; COc1n[nH]c2ncc(N3CC[C@@H](N(C)C(=O)Nc4cc(C(F)(F)F)cn(C)c4=O)[C@H](OC)C3)cc12] 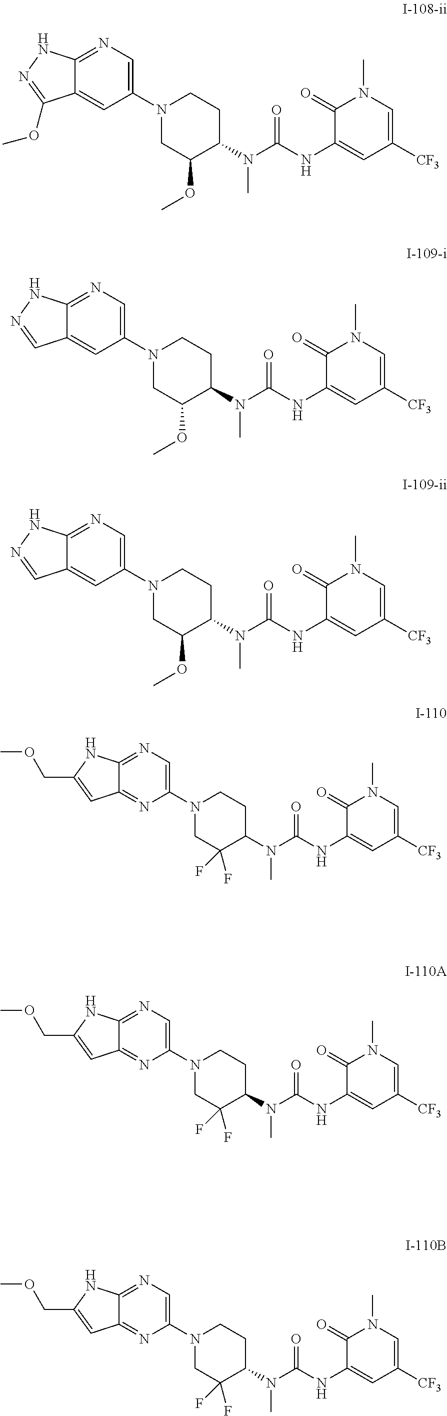 [SMILES: COc1n[nH]c2ncc(N3CC[C@H](N(C)C(=O)Nc4cc(C(F)(F)F)cn(C)c4=O)[C@@H](OC)C3)cc12 ; CO[C@@H]1CN(c2cnc3[nH]ncc3c2)CC[C@H]1N(C)C(=O)Nc1cc(C(F)(F)F)cn(C)c1=O ; CO[C@H]1CN(c2cnc3[nH]ncc3c2)CC[C@@H]1N(C)C(=O)Nc1cc(C(F)(F)F)cn(C)c1=O ; COCc1cc2nc(N3CCC(N(C)C(=O)Nc4cc(C(F)(F)F)cn(C)c4=O)C(F)(F)C3)cnc2[nH]1 ; COCc1cc2nc(N3CC[C@@H](N(C)C(=O)Nc4cc(C(F)(F)F)cn(C)c4=O)C(F)(F)C3)cnc2[nH]1 ; COCc1cc2nc(N3CC[C@H](N(C)C(=O)Nc4cc(C(F)(F)F)cn(C)c4=O)C(F)(F)C3)cnc2[nH]1] 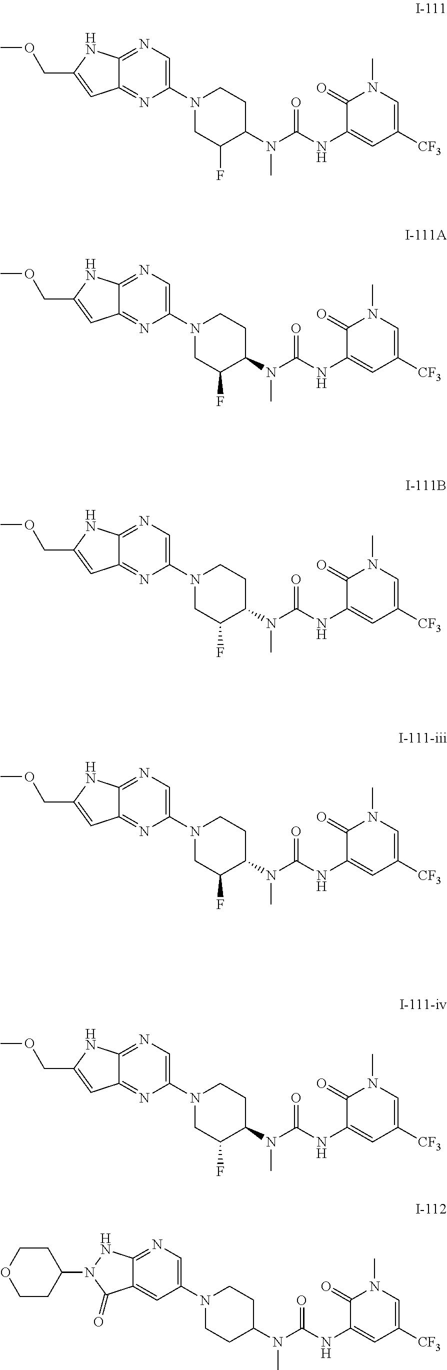 [SMILES: COCc1cc2nc(N3CCC(N(C)C(=O)Nc4cc(C(F)(F)F)cn(C)c4=O)C(F)C3)cnc2[nH]1 ; COCc1cc2nc(N3CC[C@@H](N(C)C(=O)Nc4cc(C(F)(F)F)cn(C)c4=O)[C@@H](F)C3)cnc2[nH]1 ; COCc1cc2nc(N3CC[C@H](N(C)C(=O)Nc4cc(C(F)(F)F)cn(C)c4=O)[C@H](F)C3)cnc2[nH]1 ; COCc1cc2nc(N3CC[C@H](N(C)C(=O)Nc4cc(C(F)(F)F)cn(C)c4=O)[C@@H](F)C3)cnc2[nH]1 ; COCc1cc2nc(N3CC[C@@H](N(C)C(=O)Nc4cc(C(F)(F)F)cn(C)c4=O)[C@H](F)C3)cnc2[nH]1 ; CN(C(=O)Nc1cc(C(F)(F)F)cn(C)c1=O)C1CCN(c2cnc3[nH]n(C4CCOCC4)c(=O)c3c2)CC1] 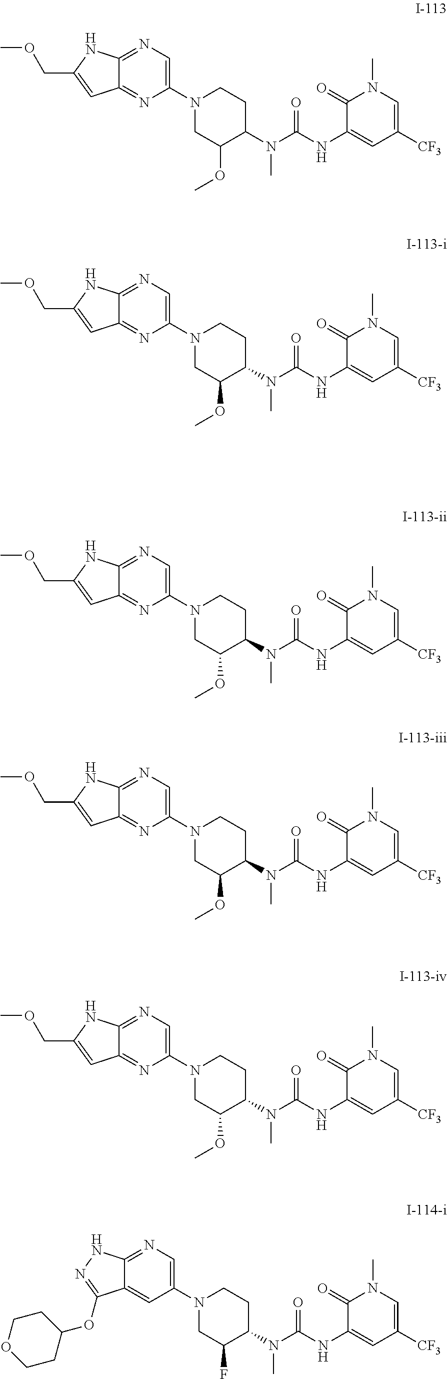 [SMILES: COCc1cc2nc(N3CCC(N(C)C(=O)Nc4cc(C(F)(F)F)cn(C)c4=O)C(OC)C3)cnc2[nH]1 ; COCc1cc2nc(N3CC[C@H](N(C)C(=O)Nc4cc(C(F)(F)F)cn(C)c4=O)[C@@H](OC)C3)cnc2[nH]1 ; COCc1cc2nc(N3CC[C@@H](N(C)C(=O)Nc4cc(C(F)(F)F)cn(C)c4=O)[C@H](OC)C3)cnc2[nH]1 ; COCc1cc2nc(N3CC[C@@H](N(C)C(=O)Nc4cc(C(F)(F)F)cn(C)c4=O)[C@@H](OC)C3)cnc2[nH]1 ; COCc1cc2nc(N3CC[C@H](N(C)C(=O)Nc4cc(C(F)(F)F)cn(C)c4=O)[C@H](OC)C3)cnc2[nH]1 ; CN(C(=O)Nc1cc(C(F)(F)F)cn(C)c1=O)[C@H]1CCN(c2cnc3[nH]nc(OC4CCOCC4)c3c2)C[C@@H]1F] 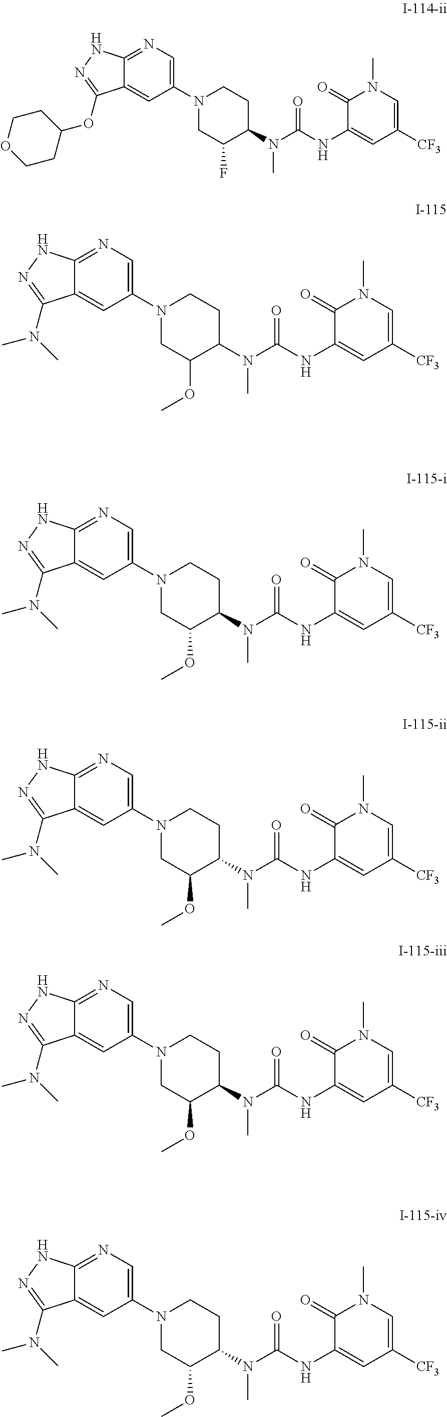 [SMILES: CN(C(=O)Nc1cc(C(F)(F)F)cn(C)c1=O)[C@@H]1CCN(c2cnc3[nH]nc(OC4CCOCC4)c3c2)C[C@H]1F ; COC1CN(c2cnc3[nH]nc(N(C)C)c3c2)CCC1N(C)C(=O)Nc1cc(C(F)(F)F)cn(C)c1=O ; CO[C@@H]1CN(c2cnc3[nH]nc(N(C)C)c3c2)CC[C@H]1N(C)C(=O)Nc1cc(C(F)(F)F)cn(C)c1=O ; CO[C@H]1CN(c2cnc3[nH]nc(N(C)C)c3c2)CC[C@@H]1N(C)C(=O)Nc1cc(C(F)(F)F)cn(C)c1=O ; CO[C@H]1CN(c2cnc3[nH]nc(N(C)C)c3c2)CC[C@H]1N(C)C(=O)Nc1cc(C(F)(F)F)cn(C)c1=O ; CO[C@@H]1CN(c2cnc3[nH]nc(N(C)C)c3c2)CC[C@@H]1N(C)C(=O)Nc1cc(C(F)(F)F)cn(C)c1=O] 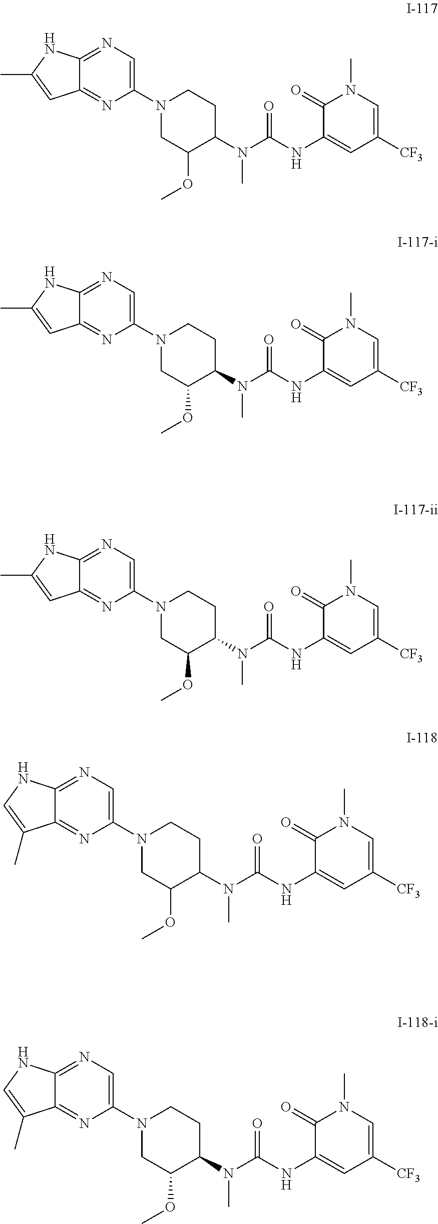 [SMILES: COC1CN(c2cnc3[nH]c(C)cc3n2)CCC1N(C)C(=O)Nc1cc(C(F)(F)F)cn(C)c1=O ; CO[C@@H]1CN(c2cnc3[nH]c(C)cc3n2)CC[C@H]1N(C)C(=O)Nc1cc(C(F)(F)F)cn(C)c1=O ; CO[C@H]1CN(c2cnc3[nH]c(C)cc3n2)CC[C@@H]1N(C)C(=O)Nc1cc(C(F)(F)F)cn(C)c1=O ; COC1CN(c2cnc3[nH]cc(C)c3n2)CCC1N(C)C(=O)Nc1cc(C(F)(F)F)cn(C)c1=O ; CO[C@@H]1CN(c2cnc3[nH]cc(C)c3n2)CC[C@H]1N(C)C(=O)Nc1cc(C(F)(F)F)cn(C)c1=O] 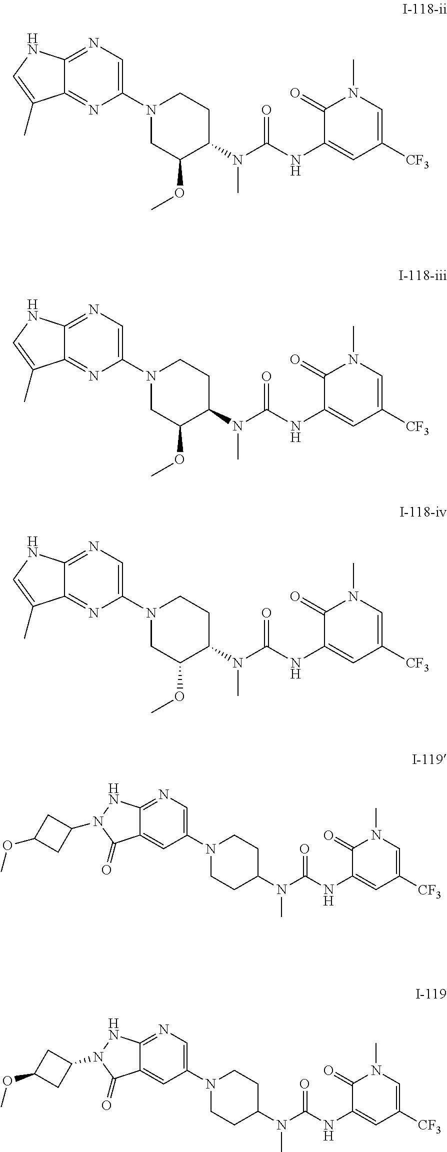 [SMILES: CO[C@H]1CN(c2cnc3[nH]cc(C)c3n2)CC[C@@H]1N(C)C(=O)Nc1cc(C(F)(F)F)cn(C)c1=O ; CO[C@H]1CN(c2cnc3[nH]cc(C)c3n2)CC[C@H]1N(C)C(=O)Nc1cc(C(F)(F)F)cn(C)c1=O ; CO[C@@H]1CN(c2cnc3[nH]cc(C)c3n2)CC[C@@H]1N(C)C(=O)Nc1cc(C(F)(F)F)cn(C)c1=O ; COC1CC(n2[nH]c3ncc(N4CCC(N(C)C(=O)Nc5cc(C(F)(F)F)cn(C)c5=O)CC4)cc3c2=O)C1 ; CO[C@H]1C[C@H](n2[nH]c3ncc(N4CCC(N(C)C(=O)Nc5cc(C(F)(F)F)cn(C)c5=O)CC4)cc3c2=O)C1] 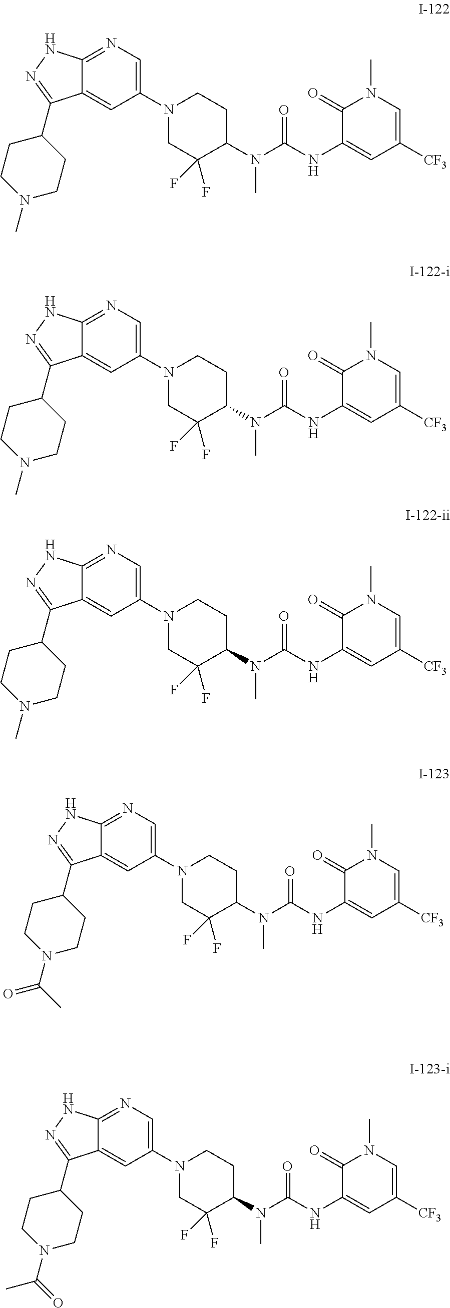 [SMILES: CN1CCC(c2n[nH]c3ncc(N4CCC(N(C)C(=O)Nc5cc(C(F)(F)F)cn(C)c5=O)C(F)(F)C4)cc23)CC1 ; CN1CCC(c2n[nH]c3ncc(N4CC[C@H](N(C)C(=O)Nc5cc(C(F)(F)F)cn(C)c5=O)C(F)(F)C4)cc23)CC1 ; CN1CCC(c2n[nH]c3ncc(N4CC[C@@H](N(C)C(=O)Nc5cc(C(F)(F)F)cn(C)c5=O)C(F)(F)C4)cc23)CC1 ; CC(=O)N1CCC(c2n[nH]c3ncc(N4CCC(N(C)C(=O)Nc5cc(C(F)(F)F)cn(C)c5=O)C(F)(F)C4)cc23)CC1 ; CC(=O)N1CCC(c2n[nH]c3ncc(N4CC[C@@H](N(C)C(=O)Nc5cc(C(F)(F)F)cn(C)c5=O)C(F)(F)C4)cc23)CC1] 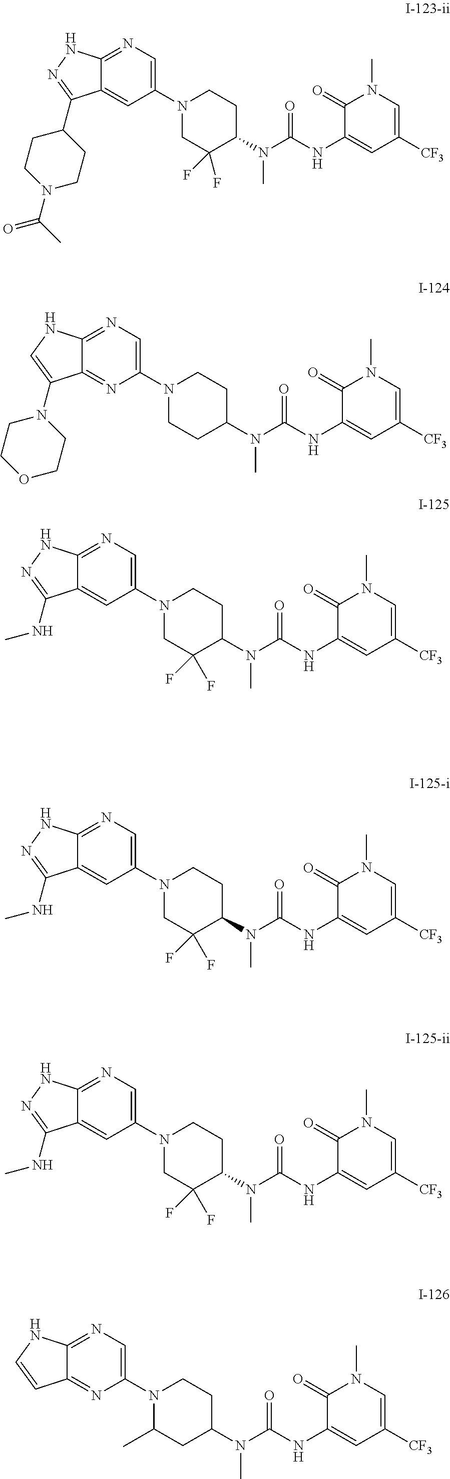 [SMILES: CC(=O)N1CCC(c2n[nH]c3ncc(N4CC[C@H](N(C)C(=O)Nc5cc(C(F)(F)F)cn(C)c5=O)C(F)(F)C4)cc23)CC1 ; CN(C(=O)Nc1cc(C(F)(F)F)cn(C)c1=O)C1CCN(c2cnc3[nH]cc(N4CCOCC4)c3n2)CC1 ; CNc1n[nH]c2ncc(N3CCC(N(C)C(=O)Nc4cc(C(F)(F)F)cn(C)c4=O)C(F)(F)C3)cc12 ; CNc1n[nH]c2ncc(N3CC[C@@H](N(C)C(=O)Nc4cc(C(F)(F)F)cn(C)c4=O)C(F)(F)C3)cc12 ; CNc1n[nH]c2ncc(N3CC[C@H](N(C)C(=O)Nc4cc(C(F)(F)F)cn(C)c4=O)C(F)(F)C3)cc12 ; CC1CC(N(C)C(=O)Nc2cc(C(F)(F)F)cn(C)c2=O)CCN1c1cnc2[nH]ccc2n1] 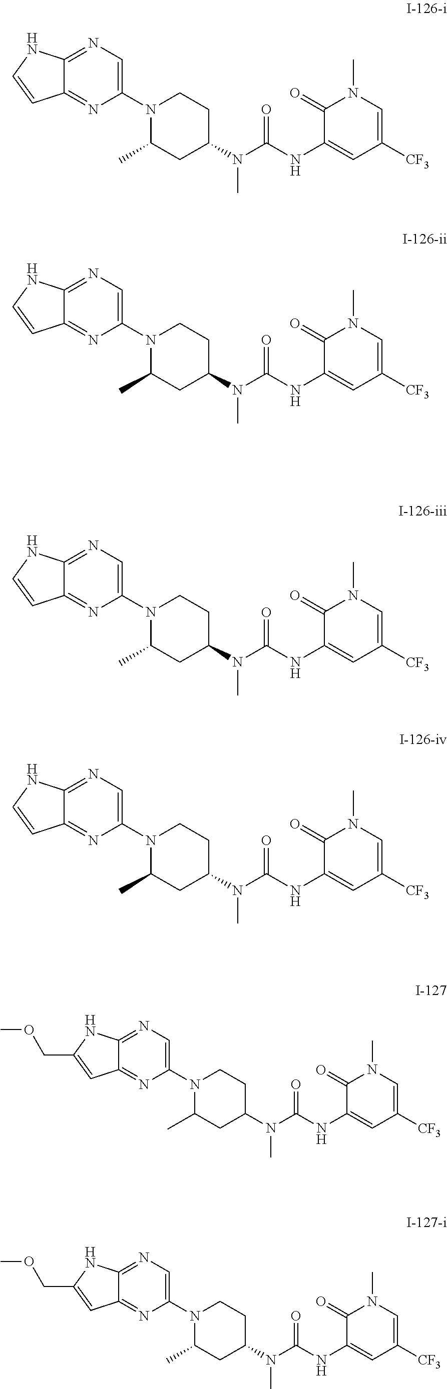 [SMILES: C[C@H]1C[C@@H](N(C)C(=O)Nc2cc(C(F)(F)F)cn(C)c2=O)CCN1c1cnc2[nH]ccc2n1 ; C[C@@H]1C[C@H](N(C)C(=O)Nc2cc(C(F)(F)F)cn(C)c2=O)CCN1c1cnc2[nH]ccc2n1 ; C[C@H]1C[C@H](N(C)C(=O)Nc2cc(C(F)(F)F)cn(C)c2=O)CCN1c1cnc2[nH]ccc2n1 ; C[C@@H]1C[C@@H](N(C)C(=O)Nc2cc(C(F)(F)F)cn(C)c2=O)CCN1c1cnc2[nH]ccc2n1 ; COCc1cc2nc(N3CCC(N(C)C(=O)Nc4cc(C(F)(F)F)cn(C)c4=O)CC3C)cnc2[nH]1 ; COCc1cc2nc(N3CC[C@H](N(C)C(=O)Nc4cc(C(F)(F)F)cn(C)c4=O)C[C@@H]3C)cnc2[nH]1] 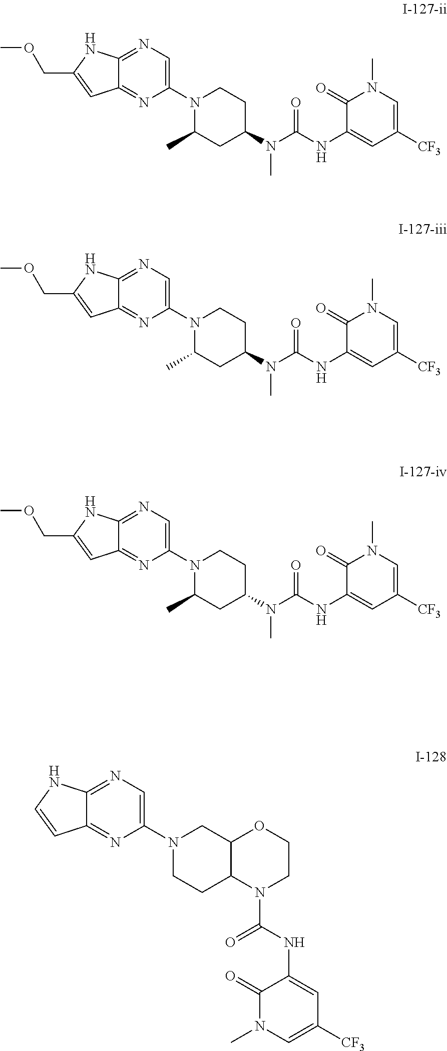 [SMILES: COCc1cc2nc(N3CC[C@@H](N(C)C(=O)Nc4cc(C(F)(F)F)cn(C)c4=O)C[C@H]3C)cnc2[nH]1 ; COCc1cc2nc(N3CC[C@@H](N(C)C(=O)Nc4cc(C(F)(F)F)cn(C)c4=O)C[C@@H]3C)cnc2[nH]1 ; COCc1cc2nc(N3CC[C@H](N(C)C(=O)Nc4cc(C(F)(F)F)cn(C)c4=O)C[C@H]3C)cnc2[nH]1 ; Cn1cc(C(F)(F)F)cc(NC(=O)N2CCOC3CN(c4cnc5[nH]ccc5n4)CCC32)c1=O] 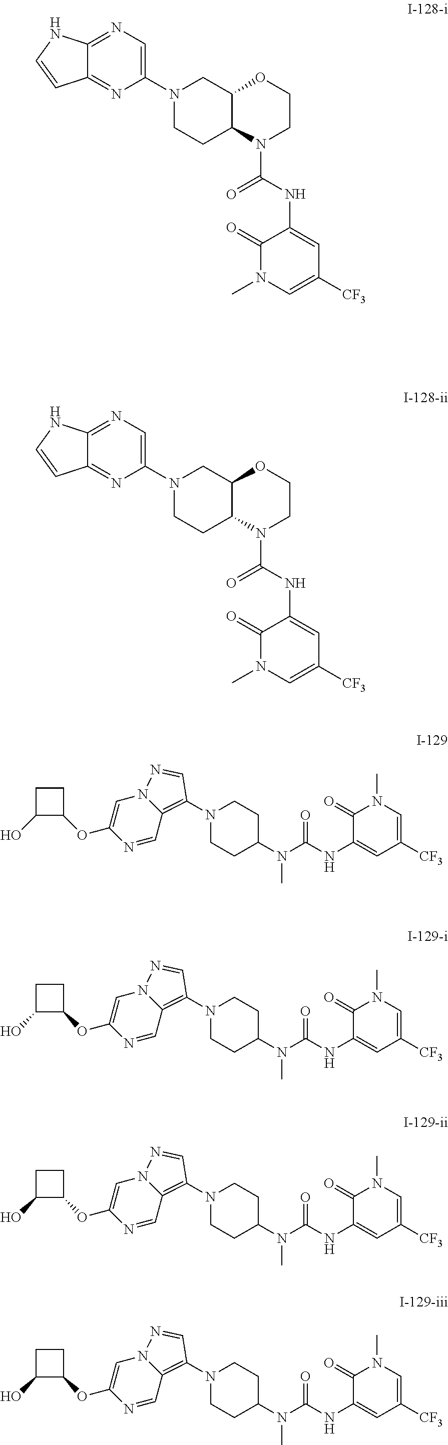 [SMILES: Cn1cc(C(F)(F)F)cc(NC(=O)N2CCO[C@H]3CN(c4cnc5[nH]ccc5n4)CC[C@@H]32)c1=O ; Cn1cc(C(F)(F)F)cc(NC(=O)N2CCO[C@@H]3CN(c4cnc5[nH]ccc5n4)CC[C@H]32)c1=O ; CN(C(=O)Nc1cc(C(F)(F)F)cn(C)c1=O)C1CCN(c2cnn3cc(OC4CCC4O)ncc23)CC1 ; CN(C(=O)Nc1cc(C(F)(F)F)cn(C)c1=O)C1CCN(c2cnn3cc(O[C@@H]4CC[C@H]4O)ncc23)CC1 ; CN(C(=O)Nc1cc(C(F)(F)F)cn(C)c1=O)C1CCN(c2cnn3cc(O[C@H]4CC[C@@H]4O)ncc23)CC1 ; CN(C(=O)Nc1cc(C(F)(F)F)cn(C)c1=O)C1CCN(c2cnn3cc(O[C@@H]4CC[C@@H]4O)ncc23)CC1] 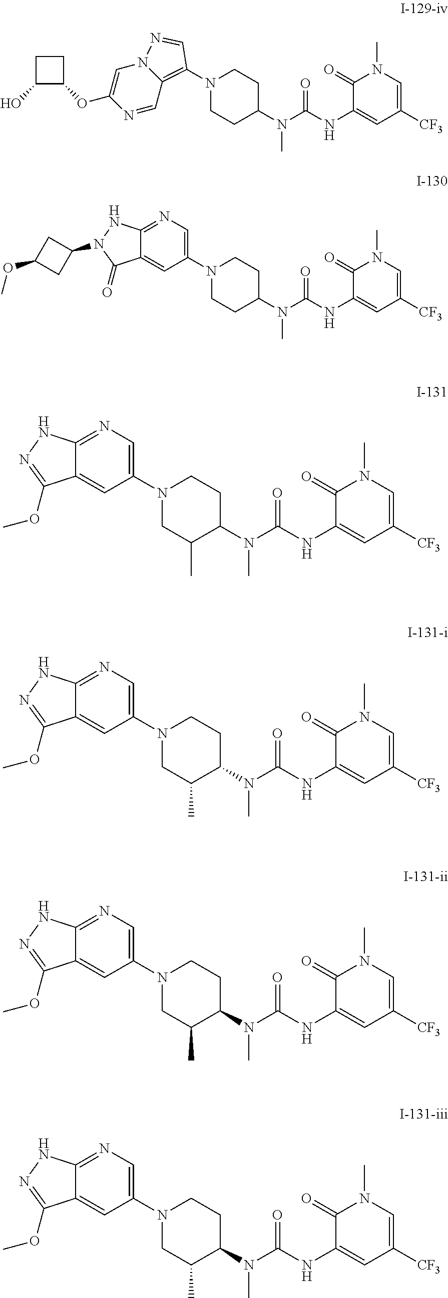 [SMILES: CN(C(=O)Nc1cc(C(F)(F)F)cn(C)c1=O)C1CCN(c2cnn3cc(O[C@H]4CC[C@H]4O)ncc23)CC1 ; CO[C@H]1C[C@@H](n2[nH]c3ncc(N4CCC(N(C)C(=O)Nc5cc(C(F)(F)F)cn(C)c5=O)CC4)cc3c2=O)C1 ; COc1n[nH]c2ncc(N3CCC(N(C)C(=O)Nc4cc(C(F)(F)F)cn(C)c4=O)C(C)C3)cc12 ; COc1n[nH]c2ncc(N3CC[C@H](N(C)C(=O)Nc4cc(C(F)(F)F)cn(C)c4=O)[C@H](C)C3)cc12 ; COc1n[nH]c2ncc(N3CC[C@@H](N(C)C(=O)Nc4cc(C(F)(F)F)cn(C)c4=O)[C@@H](C)C3)cc12 ; COc1n[nH]c2ncc(N3CC[C@@H](N(C)C(=O)Nc4cc(C(F)(F)F)cn(C)c4=O)[C@H](C)C3)cc12] 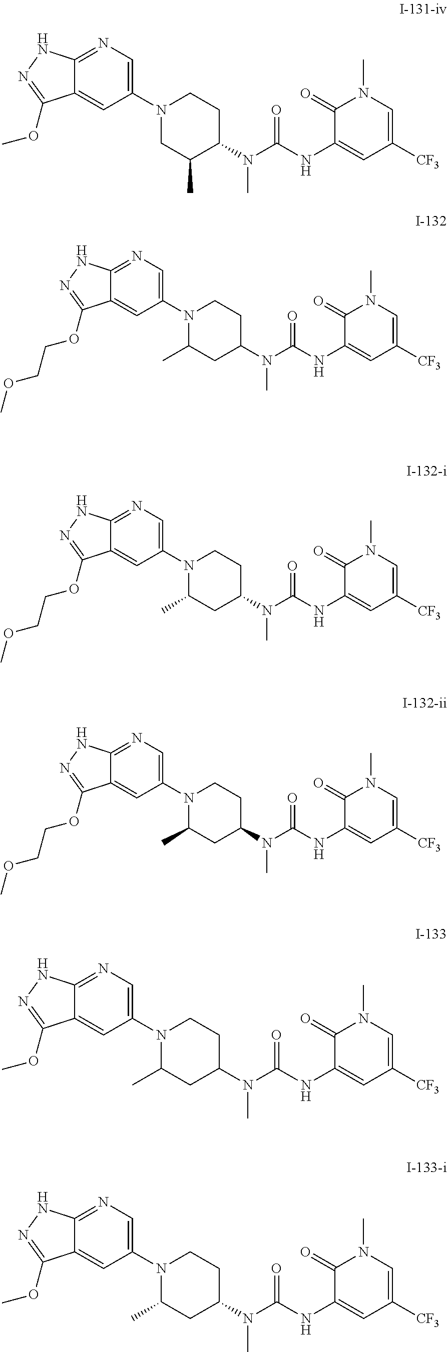 [SMILES: COc1n[nH]c2ncc(N3CC[C@H](N(C)C(=O)Nc4cc(C(F)(F)F)cn(C)c4=O)[C@@H](C)C3)cc12 ; COCCOc1n[nH]c2ncc(N3CCC(N(C)C(=O)Nc4cc(C(F)(F)F)cn(C)c4=O)CC3C)cc12 ; COCCOc1n[nH]c2ncc(N3CC[C@H](N(C)C(=O)Nc4cc(C(F)(F)F)cn(C)c4=O)C[C@@H]3C)cc12 ; COCCOc1n[nH]c2ncc(N3CC[C@@H](N(C)C(=O)Nc4cc(C(F)(F)F)cn(C)c4=O)C[C@H]3C)cc12 ; COc1n[nH]c2ncc(N3CCC(N(C)C(=O)Nc4cc(C(F)(F)F)cn(C)c4=O)CC3C)cc12 ; COc1n[nH]c2ncc(N3CC[C@H](N(C)C(=O)Nc4cc(C(F)(F)F)cn(C)c4=O)C[C@@H]3C)cc12] 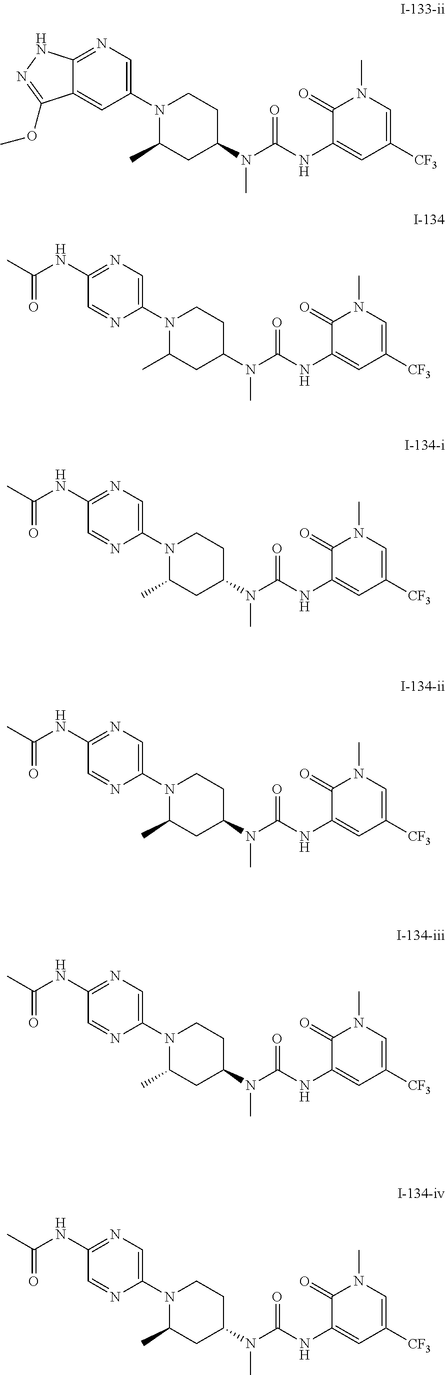 [SMILES: COc1n[nH]c2ncc(N3CC[C@@H](N(C)C(=O)Nc4cc(C(F)(F)F)cn(C)c4=O)C[C@H]3C)cc12 ; CC(=O)Nc1cnc(N2CCC(N(C)C(=O)Nc3cc(C(F)(F)F)cn(C)c3=O)CC2C)cn1 ; CC(=O)Nc1cnc(N2CC[C@H](N(C)C(=O)Nc3cc(C(F)(F)F)cn(C)c3=O)C[C@@H]2C)cn1 ; CC(=O)Nc1cnc(N2CC[C@@H](N(C)C(=O)Nc3cc(C(F)(F)F)cn(C)c3=O)C[C@H]2C)cn1 ; CC(=O)Nc1cnc(N2CC[C@@H](N(C)C(=O)Nc3cc(C(F)(F)F)cn(C)c3=O)C[C@@H]2C)cn1 ; CC(=O)Nc1cnc(N2CC[C@H](N(C)C(=O)Nc3cc(C(F)(F)F)cn(C)c3=O)C[C@H]2C)cn1] 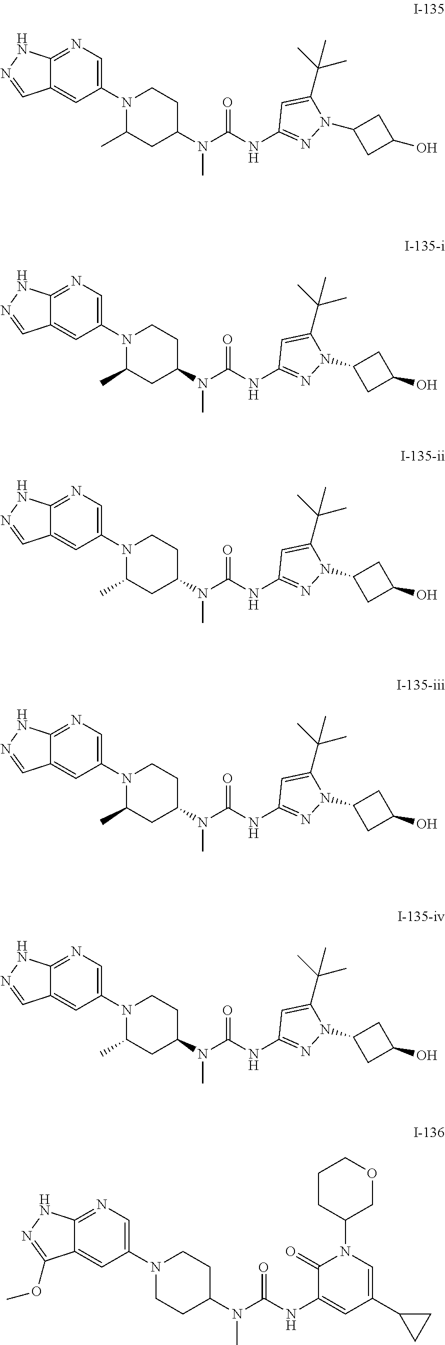 [SMILES: CC1CC(N(C)C(=O)Nc2cc(C(C)(C)C)n(C3CC(O)C3)n2)CCN1c1cnc2[nH]ncc2c1 ; C[C@@H]1C[C@H](N(C)C(=O)Nc2cc(C(C)(C)C)n([C@H]3C[C@H](O)C3)n2)CCN1c1cnc2[nH]ncc2c1 ; C[C@H]1C[C@@H](N(C)C(=O)Nc2cc(C(C)(C)C)n([C@H]3C[C@H](O)C3)n2)CCN1c1cnc2[nH]ncc2c1 ; C[C@@H]1C[C@@H](N(C)C(=O)Nc2cc(C(C)(C)C)n([C@H]3C[C@H](O)C3)n2)CCN1c1cnc2[nH]ncc2c1 ; C[C@H]1C[C@H](N(C)C(=O)Nc2cc(C(C)(C)C)n([C@H]3C[C@H](O)C3)n2)CCN1c1cnc2[nH]ncc2c1 ; COc1n[nH]c2ncc(N3CCC(N(C)C(=O)Nc4cc(C5CC5)cn(C5CCCOC5)c4=O)CC3)cc12] 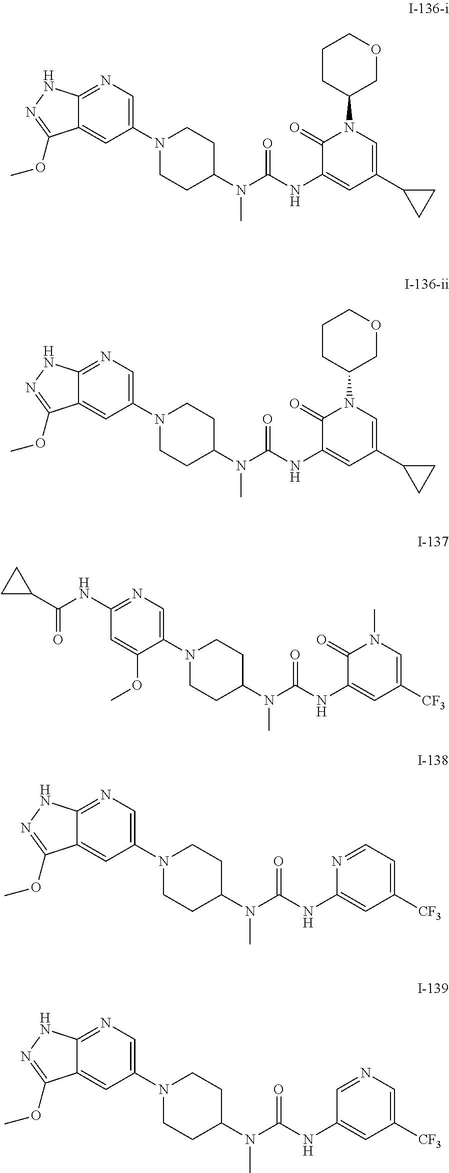 [SMILES: COc1n[nH]c2ncc(N3CCC(N(C)C(=O)Nc4cc(C5CC5)cn([C@H]5CCCOC5)c4=O)CC3)cc12 ; COc1n[nH]c2ncc(N3CCC(N(C)C(=O)Nc4cc(C5CC5)cn([C@@H]5CCCOC5)c4=O)CC3)cc12 ; COc1cc(NC(=O)C2CC2)ncc1N1CCC(N(C)C(=O)Nc2cc(C(F)(F)F)cn(C)c2=O)CC1 ; COc1n[nH]c2ncc(N3CCC(N(C)C(=O)Nc4cc(C(F)(F)F)ccn4)CC3)cc12 ; COc1n[nH]c2ncc(N3CCC(N(C)C(=O)Nc4cncc(C(F)(F)F)c4)CC3)cc12] 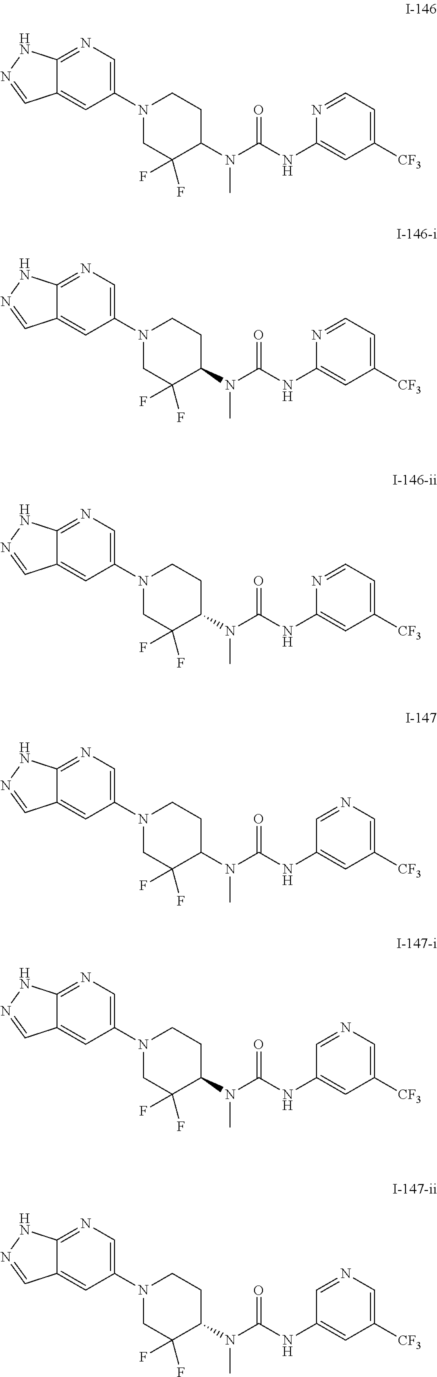 [SMILES: CN(C(=O)Nc1cc(C(F)(F)F)ccn1)C1CCN(c2cnc3[nH]ncc3c2)CC1(F)F ; CN(C(=O)Nc1cc(C(F)(F)F)ccn1)[C@@H]1CCN(c2cnc3[nH]ncc3c2)CC1(F)F ; CN(C(=O)Nc1cc(C(F)(F)F)ccn1)[C@H]1CCN(c2cnc3[nH]ncc3c2)CC1(F)F ; CN(C(=O)Nc1cncc(C(F)(F)F)c1)C1CCN(c2cnc3[nH]ncc3c2)CC1(F)F ; CN(C(=O)Nc1cncc(C(F)(F)F)c1)[C@@H]1CCN(c2cnc3[nH]ncc3c2)CC1(F)F ; CN(C(=O)Nc1cncc(C(F)(F)F)c1)[C@H]1CCN(c2cnc3[nH]ncc3c2)CC1(F)F] 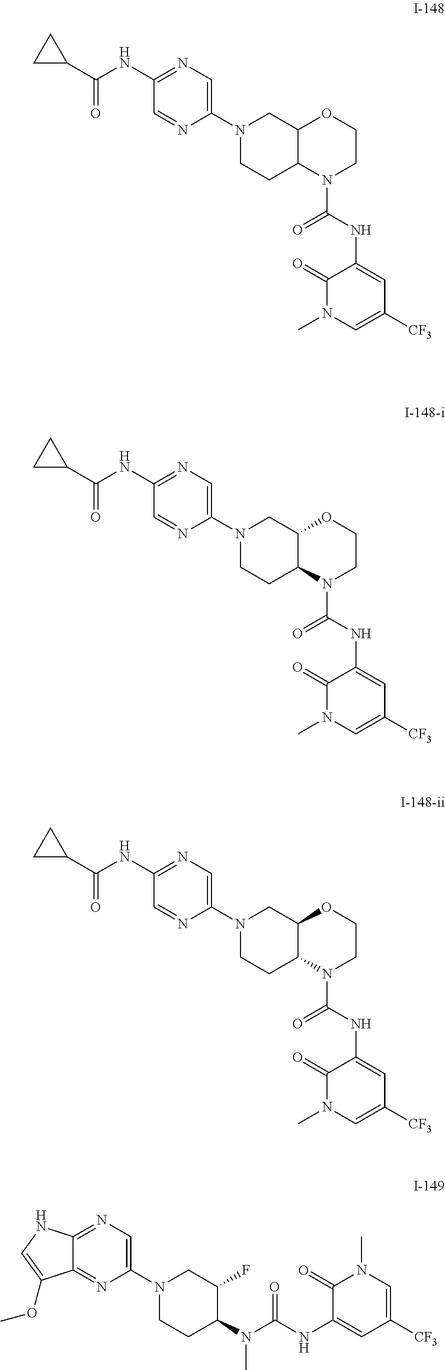 [SMILES: Cn1cc(C(F)(F)F)cc(NC(=O)N2CCOC3CN(c4cnc(NC(=O)C5CC5)cn4)CCC32)c1=O ; Cn1cc(C(F)(F)F)cc(NC(=O)N2CCO[C@H]3CN(c4cnc(NC(=O)C5CC5)cn4)CC[C@@H]32)c1=O ; Cn1cc(C(F)(F)F)cc(NC(=O)N2CCO[C@@H]3CN(c4cnc(NC(=O)C5CC5)cn4)CC[C@H]32)c1=O ; COc1c[nH]c2ncc(N3CC[C@H](N(C)C(=O)Nc4cc(C(F)(F)F)cn(C)c4=O)[C@@H](F)C3)nc12] 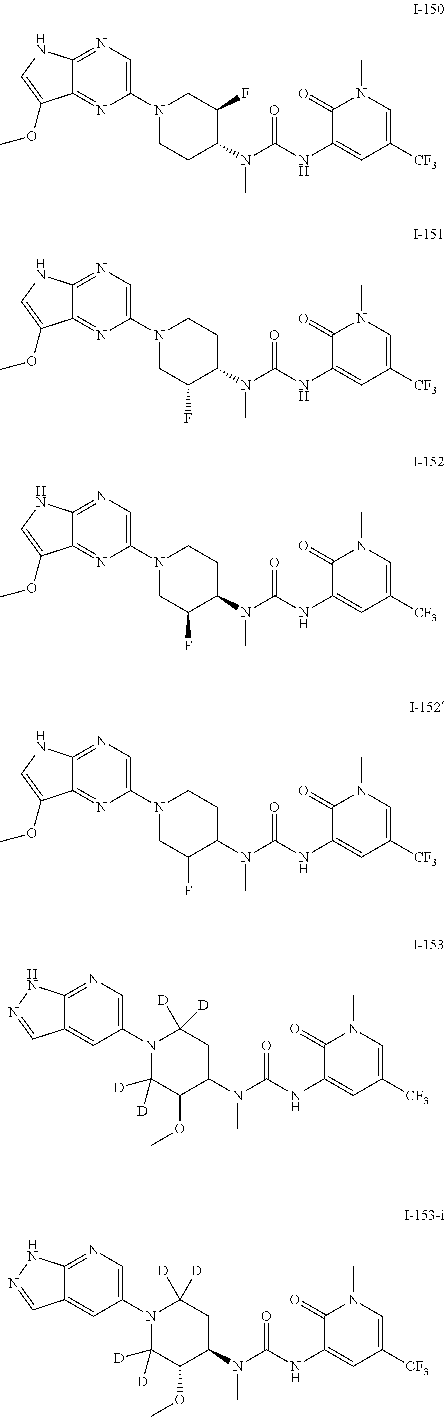 [SMILES: COc1c[nH]c2ncc(N3CC[C@@H](N(C)C(=O)Nc4cc(C(F)(F)F)cn(C)c4=O)[C@H](F)C3)nc12 ; COc1c[nH]c2ncc(N3CC[C@H](N(C)C(=O)Nc4cc(C(F)(F)F)cn(C)c4=O)[C@H](F)C3)nc12 ; COc1c[nH]c2ncc(N3CC[C@@H](N(C)C(=O)Nc4cc(C(F)(F)F)cn(C)c4=O)[C@@H](F)C3)nc12 ; COc1c[nH]c2ncc(N3CCC(N(C)C(=O)Nc4cc(C(F)(F)F)cn(C)c4=O)C(F)C3)nc12 ; [2H]C1([2H])CC(N(C)C(=O)Nc2cc(C(F)(F)F)cn(C)c2=O)C(OC)C([2H])([2H])N1c1cnc2[nH]ncc2c1 ; [2H]C1([2H])C[C@@H](N(C)C(=O)Nc2cc(C(F)(F)F)cn(C)c2=O)[C@H](OC)C([2H])([2H])N1c1cnc2[nH]ncc2c1] 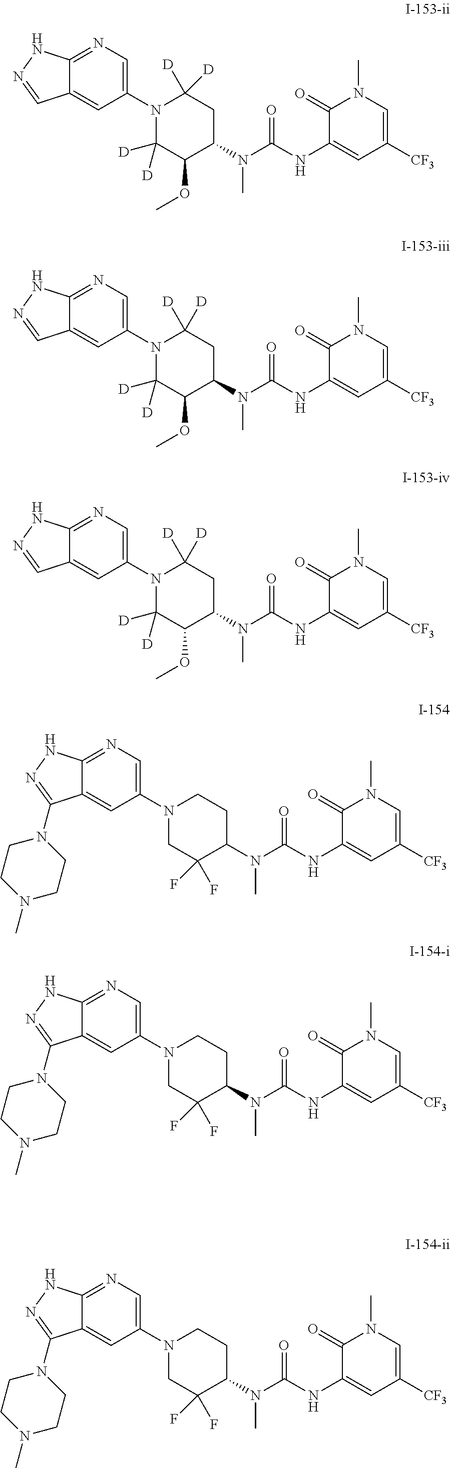 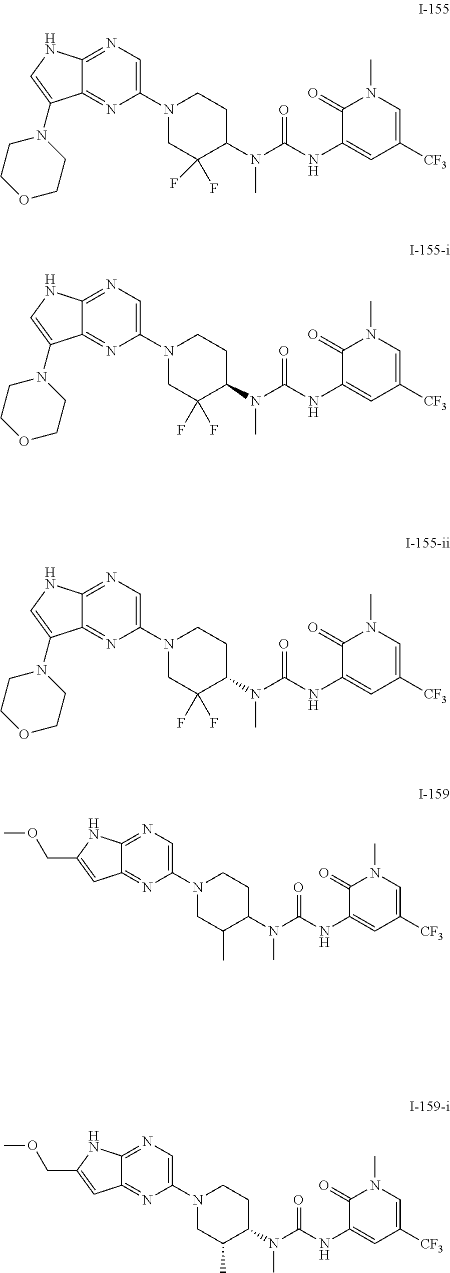 [SMILES: CN(C(=O)Nc1cc(C(F)(F)F)cn(C)c1=O)C1CCN(c2cnc3[nH]cc(N4CCOCC4)c3n2)CC1(F)F ; CN(C(=O)Nc1cc(C(F)(F)F)cn(C)c1=O)[C@@H]1CCN(c2cnc3[nH]cc(N4CCOCC4)c3n2)CC1(F)F ; CN(C(=O)Nc1cc(C(F)(F)F)cn(C)c1=O)[C@H]1CCN(c2cnc3[nH]cc(N4CCOCC4)c3n2)CC1(F)F ; COCc1cc2nc(N3CCC(N(C)C(=O)Nc4cc(C(F)(F)F)cn(C)c4=O)C(C)C3)cnc2[nH]1 ; COCc1cc2nc(N3CC[C@H](N(C)C(=O)Nc4cc(C(F)(F)F)cn(C)c4=O)[C@H](C)C3)cnc2[nH]1] 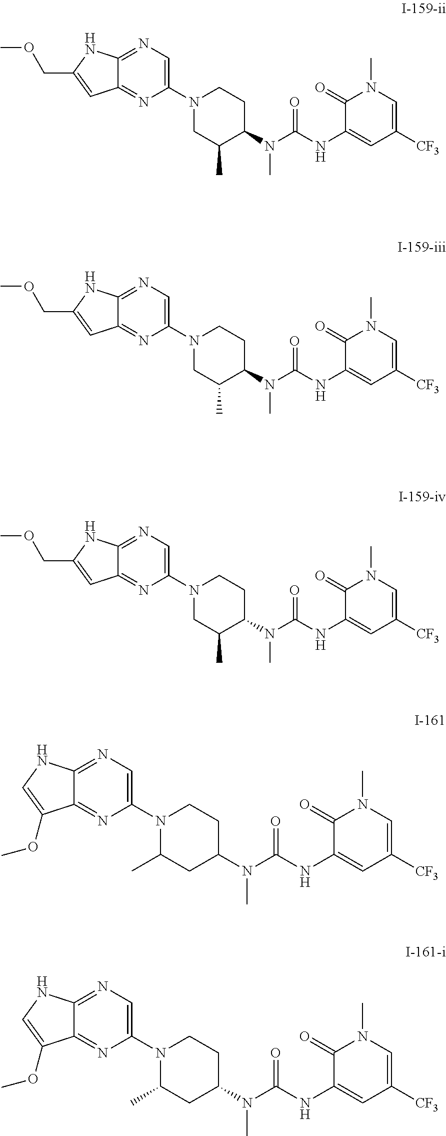 [SMILES: COCc1cc2nc(N3CC[C@@H](N(C)C(=O)Nc4cc(C(F)(F)F)cn(C)c4=O)[C@@H](C)C3)cnc2[nH]1 ; COCc1cc2nc(N3CC[C@@H](N(C)C(=O)Nc4cc(C(F)(F)F)cn(C)c4=O)[C@H](C)C3)cnc2[nH]1 ; COCc1cc2nc(N3CC[C@H](N(C)C(=O)Nc4cc(C(F)(F)F)cn(C)c4=O)[C@@H](C)C3)cnc2[nH]1 ; COc1c[nH]c2ncc(N3CCC(N(C)C(=O)Nc4cc(C(F)(F)F)cn(C)c4=O)CC3C)nc12 ; COc1c[nH]c2ncc(N3CC[C@H](N(C)C(=O)Nc4cc(C(F)(F)F)cn(C)c4=O)C[C@@H]3C)nc12] 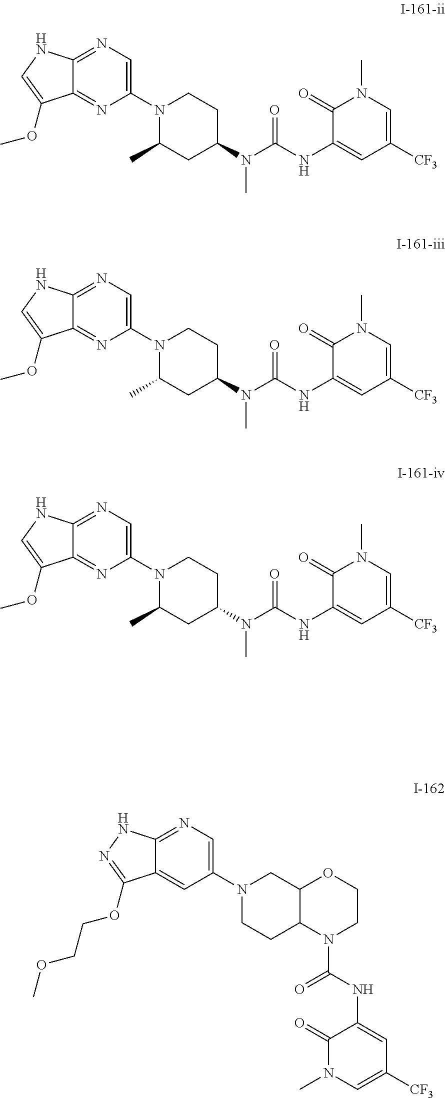 [SMILES: COc1c[nH]c2ncc(N3CC[C@@H](N(C)C(=O)Nc4cc(C(F)(F)F)cn(C)c4=O)C[C@H]3C)nc12 ; COc1c[nH]c2ncc(N3CC[C@@H](N(C)C(=O)Nc4cc(C(F)(F)F)cn(C)c4=O)C[C@@H]3C)nc12 ; COc1c[nH]c2ncc(N3CC[C@H](N(C)C(=O)Nc4cc(C(F)(F)F)cn(C)c4=O)C[C@H]3C)nc12 ; COCCOc1n[nH]c2ncc(N3CCC4C(C3)OCCN4C(=O)Nc3cc(C(F)(F)F)cn(C)c3=O)cc12] 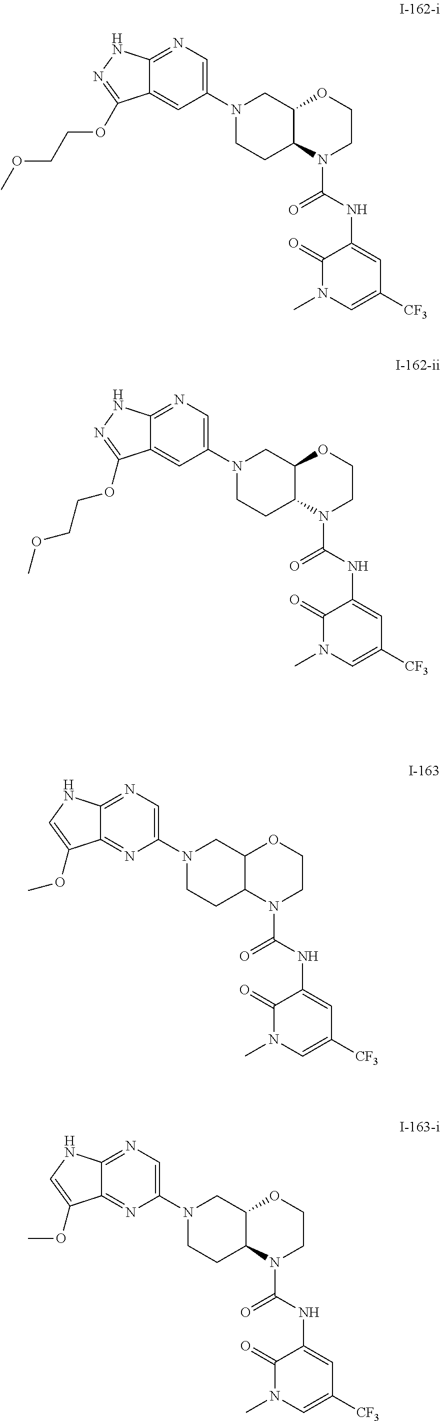 [SMILES: COCCOc1n[nH]c2ncc(N3CC[C@H]4[C@H](C3)OCCN4C(=O)Nc3cc(C(F)(F)F)cn(C)c3=O)cc12 ; COCCOc1n[nH]c2ncc(N3CC[C@@H]4[C@@H](C3)OCCN4C(=O)Nc3cc(C(F)(F)F)cn(C)c3=O)cc12 ; COc1c[nH]c2ncc(N3CCC4C(C3)OCCN4C(=O)Nc3cc(C(F)(F)F)cn(C)c3=O)nc12 ; COc1c[nH]c2ncc(N3CC[C@H]4[C@H](C3)OCCN4C(=O)Nc3cc(C(F)(F)F)cn(C)c3=O)nc12] 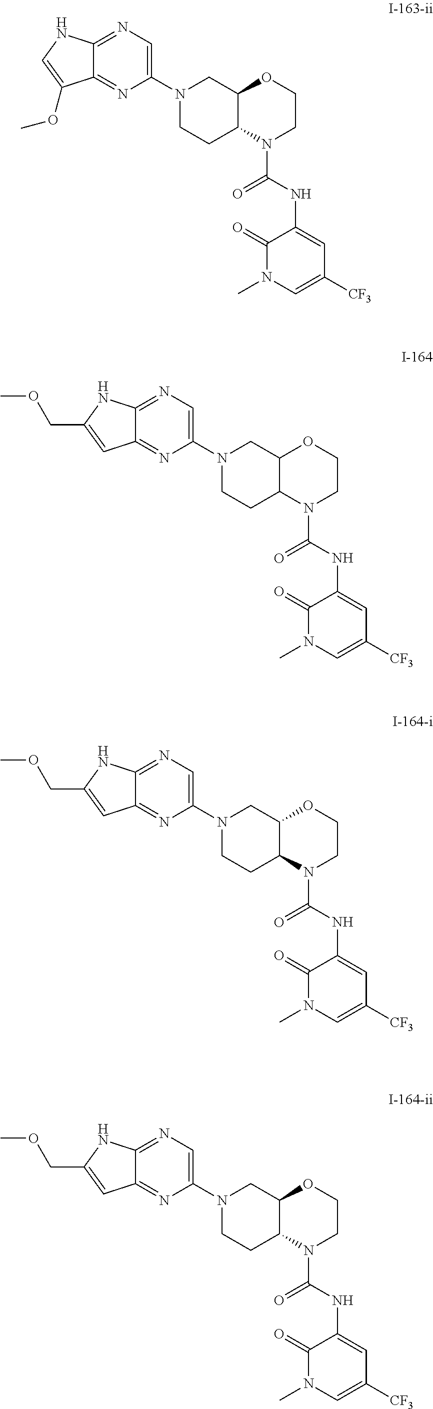 [SMILES: COc1c[nH]c2ncc(N3CC[C@@H]4[C@@H](C3)OCCN4C(=O)Nc3cc(C(F)(F)F)cn(C)c3=O)nc12 ; COCc1cc2nc(N3CCC4C(C3)OCCN4C(=O)Nc3cc(C(F)(F)F)cn(C)c3=O)cnc2[nH]1 ; COCc1cc2nc(N3CC[C@H]4[C@H](C3)OCCN4C(=O)Nc3cc(C(F)(F)F)cn(C)c3=O)cnc2[nH]1 ; COCc1cc2nc(N3CC[C@@H]4[C@@H](C3)OCCN4C(=O)Nc3cc(C(F)(F)F)cn(C)c3=O)cnc2[nH]1] 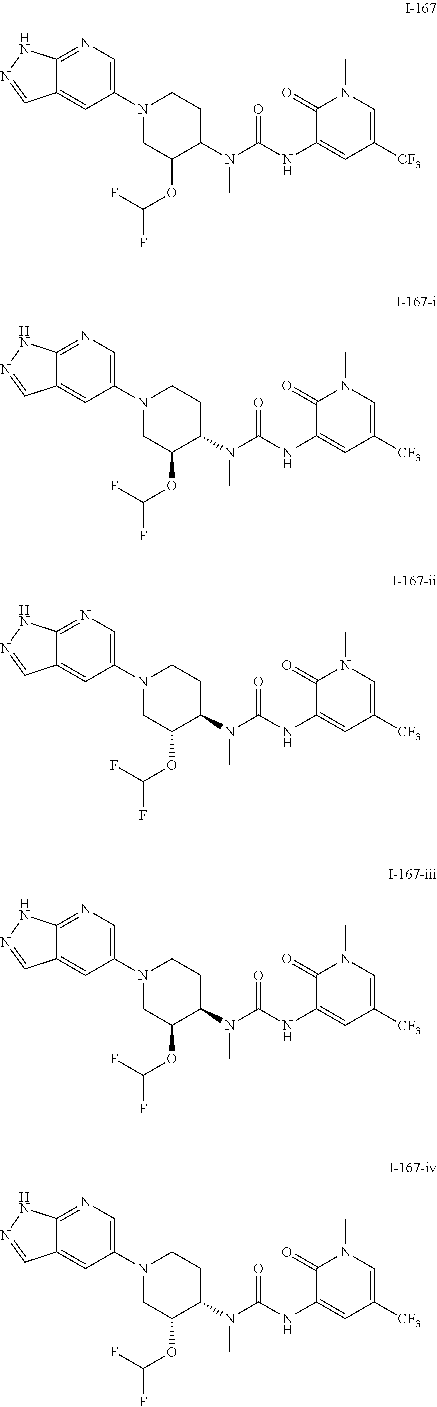 [SMILES: CN(C(=O)Nc1cc(C(F)(F)F)cn(C)c1=O)C1CCN(c2cnc3[nH]ncc3c2)CC1OC(F)F ; CN(C(=O)Nc1cc(C(F)(F)F)cn(C)c1=O)[C@H]1CCN(c2cnc3[nH]ncc3c2)C[C@@H]1OC(F)F ; CN(C(=O)Nc1cc(C(F)(F)F)cn(C)c1=O)[C@@H]1CCN(c2cnc3[nH]ncc3c2)C[C@H]1OC(F)F ; CN(C(=O)Nc1cc(C(F)(F)F)cn(C)c1=O)[C@@H]1CCN(c2cnc3[nH]ncc3c2)C[C@@H]1OC(F)F ; CN(C(=O)Nc1cc(C(F)(F)F)cn(C)c1=O)[C@H]1CCN(c2cnc3[nH]ncc3c2)C[C@H]1OC(F)F] 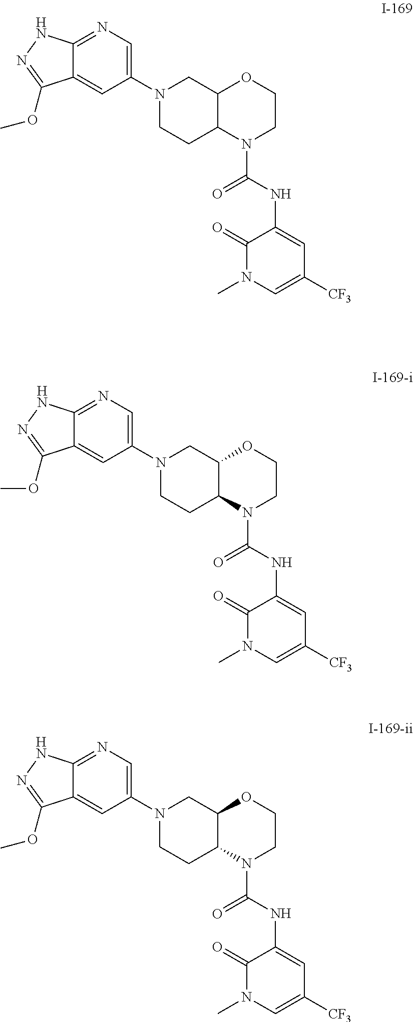 [SMILES: COc1n[nH]c2ncc(N3CCC4C(C3)OCCN4C(=O)Nc3cc(C(F)(F)F)cn(C)c3=O)cc12 ; COc1n[nH]c2ncc(N3CC[C@H]4[C@H](C3)OCCN4C(=O)Nc3cc(C(F)(F)F)cn(C)c3=O)cc12 ; COc1n[nH]c2ncc(N3CC[C@@H]4[C@@H](C3)OCCN4C(=O)Nc3cc(C(F)(F)F)cn(C)c3=O)cc12] 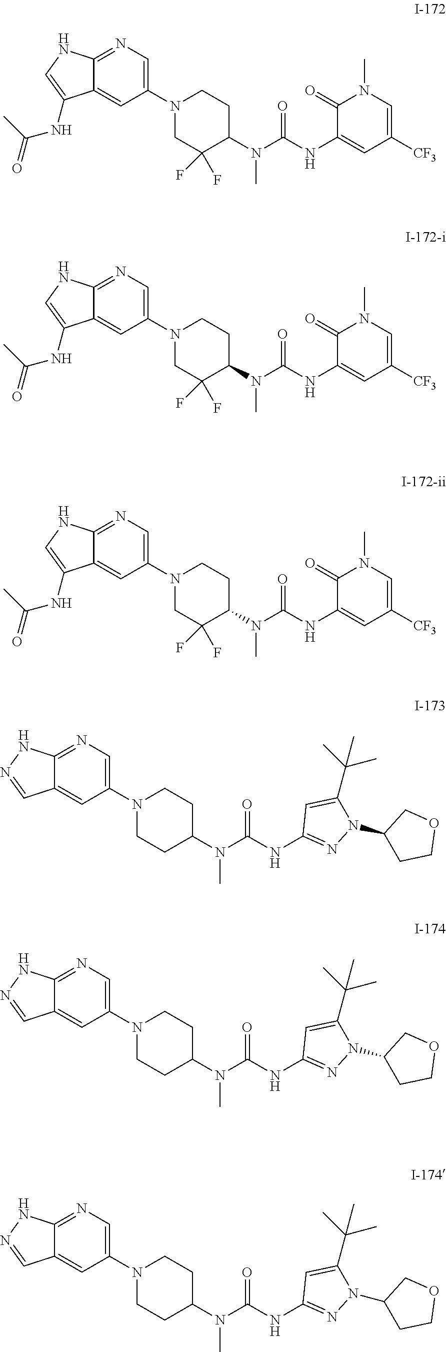 [SMILES: CC(=O)Nc1c[nH]c2ncc(N3CCC(N(C)C(=O)Nc4cc(C(F)(F)F)cn(C)c4=O)C(F)(F)C3)cc12 ; CC(=O)Nc1c[nH]c2ncc(N3CC[C@@H](N(C)C(=O)Nc4cc(C(F)(F)F)cn(C)c4=O)C(F)(F)C3)cc12 ; CC(=O)Nc1c[nH]c2ncc(N3CC[C@H](N(C)C(=O)Nc4cc(C(F)(F)F)cn(C)c4=O)C(F)(F)C3)cc12 ; CN(C(=O)Nc1cc(C(C)(C)C)n([C@@H]2CCOC2)n1)C1CCN(c2cnc3[nH]ncc3c2)CC1 ; CN(C(=O)Nc1cc(C(C)(C)C)n([C@H]2CCOC2)n1)C1CCN(c2cnc3[nH]ncc3c2)CC1 ; CN(C(=O)Nc1cc(C(C)(C)C)n(C2CCOC2)n1)C1CCN(c2cnc3[nH]ncc3c2)CC1] 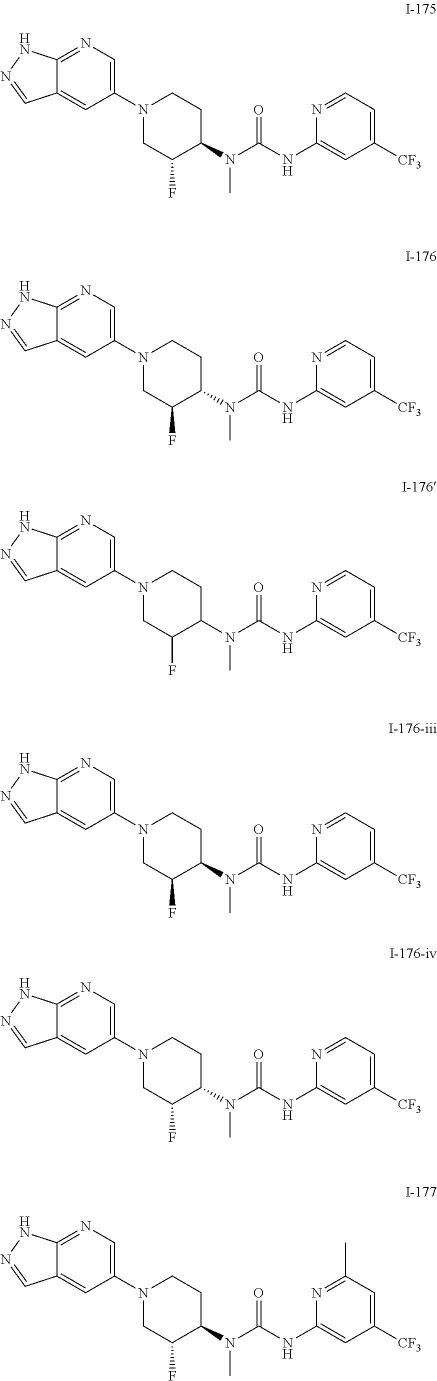 [SMILES: CN(C(=O)Nc1cc(C(F)(F)F)ccn1)[C@@H]1CCN(c2cnc3[nH]ncc3c2)C[C@H]1F ; CN(C(=O)Nc1cc(C(F)(F)F)ccn1)[C@H]1CCN(c2cnc3[nH]ncc3c2)C[C@@H]1F ; CN(C(=O)Nc1cc(C(F)(F)F)ccn1)C1CCN(c2cnc3[nH]ncc3c2)CC1F ; CN(C(=O)Nc1cc(C(F)(F)F)ccn1)[C@@H]1CCN(c2cnc3[nH]ncc3c2)C[C@@H]1F ; CN(C(=O)Nc1cc(C(F)(F)F)ccn1)[C@H]1CCN(c2cnc3[nH]ncc3c2)C[C@H]1F ; Cc1cc(C(F)(F)F)cc(NC(=O)N(C)[C@@H]2CCN(c3cnc4[nH]ncc4c3)C[C@H]2F)n1] 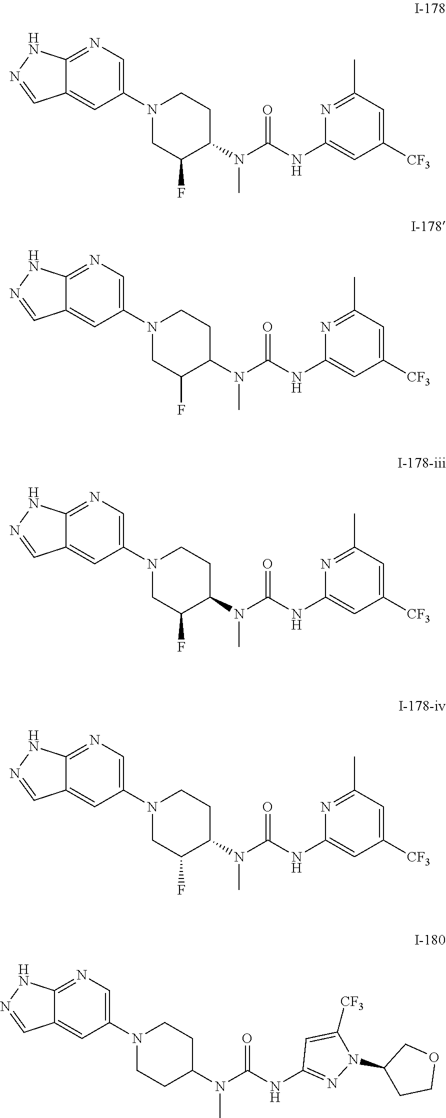 [SMILES: Cc1cc(C(F)(F)F)cc(NC(=O)N(C)[C@H]2CCN(c3cnc4[nH]ncc4c3)C[C@@H]2F)n1 ; Cc1cc(C(F)(F)F)cc(NC(=O)N(C)C2CCN(c3cnc4[nH]ncc4c3)CC2F)n1 ; Cc1cc(C(F)(F)F)cc(NC(=O)N(C)[C@@H]2CCN(c3cnc4[nH]ncc4c3)C[C@@H]2F)n1 ; Cc1cc(C(F)(F)F)cc(NC(=O)N(C)[C@H]2CCN(c3cnc4[nH]ncc4c3)C[C@H]2F)n1 ; CN(C(=O)Nc1cc(C(F)(F)F)n([C@@H]2CCOC2)n1)C1CCN(c2cnc3[nH]ncc3c2)CC1] 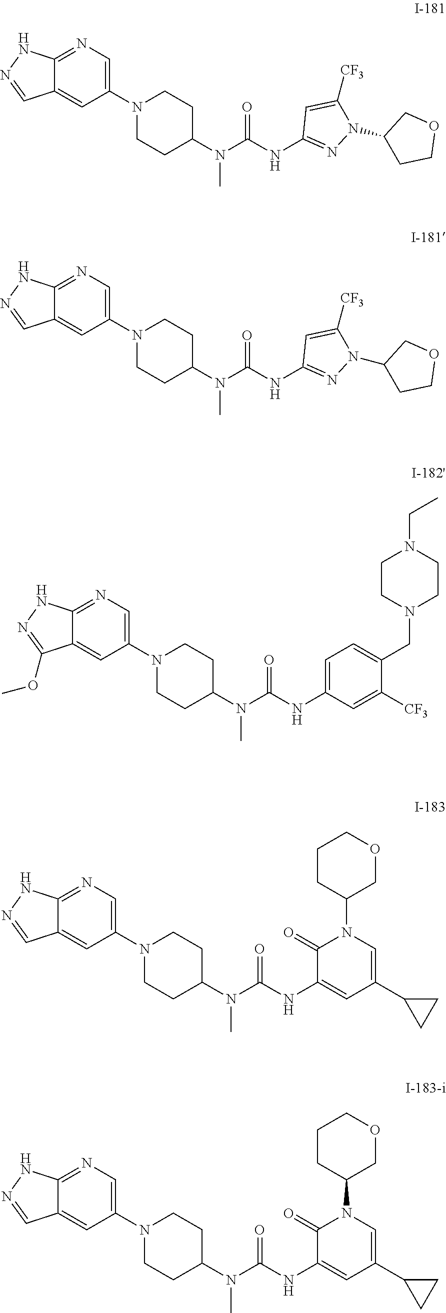 [SMILES: CN(C(=O)Nc1cc(C(F)(F)F)n([C@H]2CCOC2)n1)C1CCN(c2cnc3[nH]ncc3c2)CC1 ; CN(C(=O)Nc1cc(C(F)(F)F)n(C2CCOC2)n1)C1CCN(c2cnc3[nH]ncc3c2)CC1 ; CCN1CCN(Cc2ccc(NC(=O)N(C)C3CCN(c4cnc5[nH]nc(OC)c5c4)CC3)cc2C(F)(F)F)CC1 ; CN(C(=O)Nc1cc(C2CC2)cn(C2CCCOC2)c1=O)C1CCN(c2cnc3[nH]ncc3c2)CC1 ; CN(C(=O)Nc1cc(C2CC2)cn([C@H]2CCCOC2)c1=O)C1CCN(c2cnc3[nH]ncc3c2)CC1] 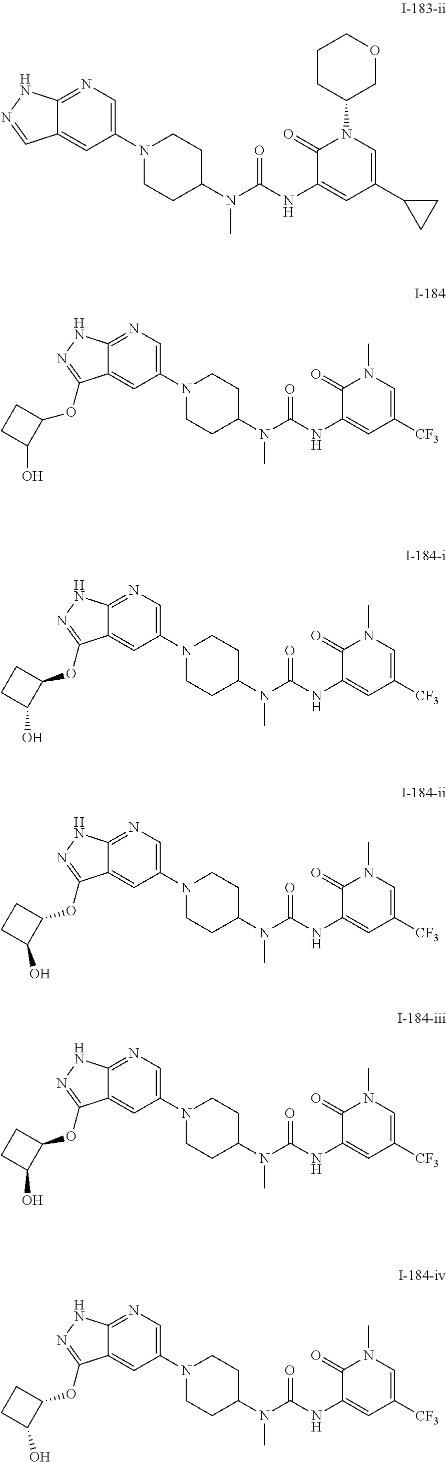 [SMILES: CN(C(=O)Nc1cc(C2CC2)cn([C@@H]2CCCOC2)c1=O)C1CCN(c2cnc3[nH]ncc3c2)CC1 ; CN(C(=O)Nc1cc(C(F)(F)F)cn(C)c1=O)C1CCN(c2cnc3[nH]nc(OC4CCC4O)c3c2)CC1 ; CN(C(=O)Nc1cc(C(F)(F)F)cn(C)c1=O)C1CCN(c2cnc3[nH]nc(O[C@@H]4CC[C@H]4O)c3c2)CC1 ; CN(C(=O)Nc1cc(C(F)(F)F)cn(C)c1=O)C1CCN(c2cnc3[nH]nc(O[C@H]4CC[C@@H]4O)c3c2)CC1 ; CN(C(=O)Nc1cc(C(F)(F)F)cn(C)c1=O)C1CCN(c2cnc3[nH]nc(O[C@@H]4CC[C@@H]4O)c3c2)CC1 ; CN(C(=O)Nc1cc(C(F)(F)F)cn(C)c1=O)C1CCN(c2cnc3[nH]nc(O[C@H]4CC[C@H]4O)c3c2)CC1]
or a pharmaceutically acceptable salt thereof.

68. A pharmaceutical composition comprising the compound of claim 1, or a pharmaceutically acceptable salt thereof, and a pharmaceutically acceptable carrier.

69. A method of inhibiting JAK2 in a subject comprising administering the compound of claim 1.

70. A method of treating a disease, disorder, or condition associated with JAK2, comprising administering to a subject in need thereof the compound of claim 1.

71. A method of treating cancer, comprising administering to a subject in need thereof the compound of claim 1.

72-75. (canceled)
